(19) United States
(12) Patent Application Publication
(10) Pub. No.: US 2026/0070901 A1
(43) Pub. Date: Mar. 12, 2026

(54) NOVEL COMPOUNDS AS CK2 INHIBITORS

(71) Applicant: CAMBRIDGE ENTERPRISE LIMITED, aa (GB)

(72) Inventors: Paul Brear, Cambridgeshire (GB); Marko Juhana Hyvonen, Cambridgeshire (GB); Paul Glossop, Cambridgeshire (GB); David Robert Spring, Cambridgeshire (GB)

(21) Appl. No.: 19/109,285

(22) PCT Filed: Sep. 8, 2023
(86) PCT No.: PCT/GB23/52327

§ 371 Date: Mar. 6, 2025

(30) Foreign Application Priority Data
Aug. 9, 2022 (GB) 2213166.8

(51) Int. Cl. C07D 471/04; A61K 31/4745; A61K 31/496; A61K 31/497; A61K 31/506; A61K 31/519; C07D 487/04

(52) U.S. Cl. CPC C07D 471/04; A61K 31/4745; A61K 31/496; A61K 31/497; A61K 31/506; A61K 31/519; C07D 487/04

(57) ABSTRACT

Provided are compounds of the Formula I, and salts, hydrates and solvates thereof:

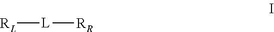

wherein R_{L}, L and R_{R} are each as defined in the specification. The compounds are inhibitors of Casein Kinase 2 alpha (CK2α) and are useful for the treatment and/or prevention of diseases and conditions in which CK2α activity is implicated, such as, for example, but not limited to, the treatment and/or prevention of proliferative disorders (e.g. cancer), viral infections, inflammation, diabetes, vascular and ischemic disorders, neurodegeneration and the regulation of circadian rhythm. The present invention also relates to pharmaceutical compositions comprising the compounds defined herein, to processes for synthesizing these compounds and to their use for the treatment of diseases and/or conditions in which CK2α activity is implicated.

### INTRODUCTION

The present invention relates to novel therapeutic compounds. More specifically, the present invention relates to novel therapeutic compounds that inhibit Casein Kinase 2 alpha subunit (CK2α (CSNK2A1) and/or CK2α′ (CSNK2A2)) and as part of the CK2 holoenzyme. The novel therapeutic compounds are therefore useful for the treatment and/or prevention of diseases and conditions in which CK2α activity is implicated, such as, for example but not limited to, the treatment and/or prevention of proliferative disorders (e.g. cancer), viral infections, inflammation, diabetes, vascular and ischemic disorders, neurodegeneration and the regulation of circadian rhythm.

The present invention also relates to pharmaceutical compositions comprising the novel therapeutic compounds defined herein, to processes for synthesising these compounds and to their use for the treatment of diseases and/or conditions in which CK2α activity is implicated.

### BACKGROUND OF THE INVENTION

CK2α is a serine/threonine kinase that is a key regulator of many cellular processes and is involved in cellular proliferation and anti-apoptotic mechanisms (Battistutta & Lolli, Mol. Cell. Biochem. 2011). It mainly exists as a holoenzyme composed of two catalytic (α and/or α′) and a dimer of regulatory (β) subunits, but it can also be found as the isolated subunits (Niefind et al, EMBO J 2001). Unlike most other kinases, it is constitutively active and more than 300 proteins have been identified as putative CK2α substrates, making it one of the most pleiotropic proteins in eukaryotic systems (Meggio & Pinna, FASEB 2003).

CK2α is a pro-survival kinase that operates across multiple signaling pathways to convey a proliferative and anti-apoptotic phenotype to cells. Consequently, cancer cells are often described as being addicted to CK2α activity and a high-profile genome-wide CRISPR-Cas9 screen highlighted CK2α as a top tier, high priority drug target for Colorectal Cancer (CRC) (Behan et al, Nature 2019). The target is well validated by human data that correlates poor patient survival in numerous tumor types, including CRC, with increased CK2α expression (Lin et al, PLoS ONE 2011). Additionally, data from clinical samples shows CK2α expression is upregulated in numerous tumor types (Ortega et al, PLoS ONE 2014; Di Maira et al, 2019).

The human genetics of CRC are well characterized and approximately 80% tumors are identified as being wnt pathway mutation driven (e.g. APC, β-catenin) (Zhan et al, Oncogene 2017). The wnt pathway is known to be sensitive to and amplified by CK2α activity and can be inhibited by loss of CK2α function (Gao & Wang, J B C 2006). For example, in animal models, CK2α inhibition prevents tumor growth that is driven by different mutations in the wnt pathway (Dowling et al, ACS 2016).

CK2α also contributes to the malignant phenotype in cholangiocarcinoma (CCA), which is known to be a wnt-dysregulated tumor type (Zhan et al, Oncogene 2017). CK2α is over-expressed in human CCA samples and CCA tumor cell lines (Di Maira et al, Oncogenesis 2019); and disruption of CK2α activity in CCA cell models is reported to inhibit tumorigenic properties (Zakharia et al, Translational Oncology 2019).

It is hypothesised that a CK2α inhibitor given either as a monotherapy, in combination with standard of care chemotherapy or in combination with other targeted therapies in development, such as, but not limited to, KRAS inhibitors, will inhibit CRC tumor growth by reversing aberrant upregulation of wnt signaling to restore the normal balance of apoptosis and proliferation.

Existing CK2α inhibitors target the highly conserved ATP binding site. This design strategy often leads to a poor selectivity profile for such inhibitors over other kinases. There is therefore a need for potent and more selective CK2α inhibitors that bind to the catalytic ATP site of CK2α (to drive potent enzyme inhibition) but also interact with other areas of CK2α, such as the αD site (to drive high levels of selectivity over other kinases).

The present invention was devised with the foregoing in mind.

### SUMMARY OF THE INVENTION

In one aspect, the present invention provides a compound of Formula I as defined herein, and/or a pharmaceutically acceptable salt, hydrate or solvate thereof.

In another aspect, the present invention provides a pharmaceutical composition which comprises a compound of Formula I as defined herein, or a pharmaceutically acceptable salt, hydrate or solvate thereof, and one or more pharmaceutically acceptable excipients.

In another aspect, the present invention provides a compound of Formula I as defined herein, or a pharmaceutically acceptable salt, hydrate or solvate thereof, or a pharmaceutical composition as defined herein, for use in therapy.

In another aspect, the present invention provides a compound of Formula I as defined herein, or a pharmaceutically acceptable salt, hydrate or solvate thereof, or a pharmaceutical composition as defined herein, for use in the treatment of a disease or condition in which CK2α activity is implicated.

In another aspect, the present invention provides a compound of Formula I as defined herein, or a pharmaceutically acceptable salt, hydrate or solvate thereof, or a pharmaceutical composition as defined herein, for use in the treatment of a disease or condition associated with aberrant activity of CK2α.

In another aspect, the present invention provides a compound of Formula I as defined herein, or a pharmaceutically acceptable salt, hydrate or solvate thereof, or a pharmaceutical composition as defined herein, for use in the treatment of proliferative disorders (e.g. cancer or benign neoplasms), viral infections, an inflammatory disease or condition, diabetes, vascular and ischemic disorders, neurodegenerative disorders and/or the regulation of circadian rhythm.

In another aspect, the present invention provides a compound of Formula I as defined herein, or a pharmaceutically acceptable salt, hydrate or solvate thereof, or a pharmaceutical composition as defined herein, for use in the treatment of a cancer.

In another aspect, the present invention provides a compound of Formula I as defined herein, or a pharmaceutically acceptable salt, hydrate or solvate thereof, or a pharmaceutical composition as defined herein, for use in the treatment of a viral infection.

In another aspect, the present invention provides the use of a compound of Formula I as defined herein, or a pharmaceutically acceptable salt, hydrate or solvate thereof, in the manufacture of a medicament for use in the treatment of a disease or condition in which CK2α activity is implicated.

In another aspect, the present invention provides the use of a compound of Formula I as defined herein, or a pharmaceutically acceptable salt, hydrate or solvate thereof, in the manufacture of a medicament for use in the treatment of a disease or condition associated with aberrant activity of CK2α.

In another aspect, the present invention provides the use of a compound of Formula I as defined herein, or a pharmaceutically acceptable salt, hydrate or solvate thereof, in the manufacture of a medicament for use in the treatment of proliferative disorders (e.g. cancer or benign neoplasms), viral infections, an inflammatory disease or condition, diabetes, vascular and ischemic disorders, neurodegenerative disorders and/or the regulation of circadian rhythm.

In another aspect, the present invention provides the use of a compound of Formula I as defined herein, or a pharmaceutically acceptable salt, hydrate or solvate thereof, in the manufacture of a medicament for use in the treatment of a cancer.

In another aspect, the present invention provides the use of a compound of Formula I as defined herein, or a pharmaceutically acceptable salt, hydrate or solvate thereof, in the manufacture of a medicament for use in the treatment of a viral infection.

In another aspect, the present invention provides a method of treating a disease or condition in which CK2α activity is implicated, said method comprising administering to a subject in need thereof an effective amount of a compound of Formula I as defined herein, or a pharmaceutically acceptable salt, hydrate or solvate thereof, or a pharmaceutical composition as defined herein.

In another aspect, the present invention provides a method of treating a disease or condition associated with aberrant activity of CK2α, said method comprising administering to a subject in need thereof an effective amount of a compound of Formula I as defined herein, or a pharmaceutically acceptable salt, hydrate or solvate thereof, or a pharmaceutical composition as defined herein.

In another aspect, the present invention provides a method of treating a proliferative disorder (e.g. cancer or benign neoplasms), a viral infection, an inflammatory disease or condition, diabetes, vascular and ischemic disorders, neurodegenerative disorders and/or regulating cardiac rhythm, said method comprising administering to a subject in need thereof an effective amount of a compound of Formula I as defined herein, or a pharmaceutically acceptable salt, hydrate or solvate thereof, or a pharmaceutical composition as defined herein.

In another aspect, the present invention provides a method of treating cancer, said method comprising administering to a subject in need thereof an effective amount of a compound of Formula I as defined herein, or a pharmaceutically acceptable salt, hydrate or solvate thereof, or a pharmaceutical composition as defined herein.

In another aspect, the present invention provides a method of treating a viral infection, said method comprising administering to a subject in need thereof an effective amount of a compound of Formula I as defined herein, or a pharmaceutically acceptable salt, hydrate or solvate thereof, or a pharmaceutical composition as defined herein.

In another aspect, the present invention provides a combination treatment comprising a compound of Formula I, or a pharmaceutically acceptable salt, hydrate or solvate thereof, as defined herein, with one or more additional therapeutic agents.

In another aspect, the present invention provides processes for preparing compounds of Formula I as defined herein, or a pharmaceutically acceptable salt, hydrate or solvate thereof, as defined herein, with one or more additional therapeutic agents.

Preferred, suitable, and optional features of any one particular aspect of the present invention are also preferred, suitable, and optional features of any other aspect.

### DETAILED DESCRIPTION OF THE INVENTION

### Definitions

Unless otherwise stated, the following terms used in the specification and claims have the following meanings set out below.

It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

References to “Casein Kinase 2 alpha” or “CK2α” herein include CK2α (CSNK2A1) and/or CK2α′ (CSNK2A2). Where reference is made to the compounds of the present invention defined herein inhibiting CK2α or being CK2α inhibitors, we mean that the compounds function as inhibitors of CK2α (CSNK2A1) and/or CK2α′ (CSNK2A2) and the CK2 holoenzyme. In a particular embodiment, the compounds of the invention inhibit CK2α (CSNK2A1). In another embodiment, the compounds of the invention inhibit CK2α′ (CSNK 2A2).

The compounds and intermediates described herein may be named according to either the IUPAC (International Union for Pure and Applied Chemistry) or CAS (Chemical Abstracts Service) nomenclature systems. It should be understood that unless expressly stated to the contrary, the terms “compounds of Formula I”, “compounds of the invention” and the more general term “compounds” refer to and include any and all compounds described by and/or with reference to Formula I herein. It should also be understood that these terms encompass all stereoisomers, i.e. cis and trans isomers, as well as optical isomers, i.e. R and S enantiomers, of such compounds, in substantially pure form and/or any mixtures of the foregoing in any ratio. This understanding extends to pharmaceutical compositions and methods of treatment that employ or comprise one or more compounds of the Formula I, either by themselves or in combination with additional agents.

Unless specified otherwise, atoms are referred to herein by their chemical symbol as appearing in the IUPAC periodic table of the Elements. For example, “C” refers to a carbon atom.

The term “(m-nC)” or “(m-nC) group” used alone or as a prefix, refers to any group having m to n carbon atoms.

In this specification the term “alkyl” includes both straight and branched chain alkyl groups. References to individual alkyl groups such as “propyl” are specific for the straight chain version only and references to individual branched chain alkyl groups such as “isopropyl” are specific for the branched chain version only. For Example, “(1-6C)alkyl” includes (1-4C)alkyl, (1-3C)alkyl, propyl, isopropyl and t-butyl. A similar convention applies to other radicals, for example “phenyl(1-6C)alkyl” includes phenyl(1-4C)alkyl, benzyl, 1-phenylethyl and 2-phenylethyl.

An “alkylene” group is an alkyl group that is positioned between and serves to connect two other chemical groups. Thus, “(1-6C)alkylene” means a linear saturated divalent hydrocarbon radical of one to six carbon atoms or a branched saturated divalent hydrocarbon radical of three to six carbon atoms, for example, methylene, ethylene, propylene, 2-methylpropylene, pentylene, and the like.

“(3-6C)cycloalkyl” means a hydrocarbon ring containing from 3 to 6 carbon atoms, for example, cyclopropyl, cyclobutyl, cyclopentyl or cyclohexyl.

“(3-6C)cycloalkoxy” refers to cycloalkoxy groups (i.e. O-cycloalkyl group) wherein the cycloalkyl group means a hydrocarbon ring containing from 3 to 6 carbon atoms, for example, —O-cyclopropyl, —O-cyclobutyl, —O-cyclopentyl or —O-cyclohexyl.

The term “halo”, “halogen” or “halogeno” refers to fluoro, chloro, bromo and iodo.

As used herein by themselves or in conjunction with another term or terms, “haloalkyl” and “haloalkyl group” refer to alkyl groups in which one or more hydrogen atoms are replaced by halogen atoms. Representative examples include, but are not limited to, —CF₃, —CHF₂, —CH₂F, —CF₂CF₃, —CHFCF₃, and —CH₂CF₃. Suitably, a haloalkyl group is selected from —CHF₂ and —CF₃, suitably —CF₃.

As used herein by themselves or in conjunction with another term or terms, “haloalkoxy” and “haloalkoxy group” refer to alkoxy groups (i.e. O-alkyl groups) in which one or more hydrogen atoms are replaced by halogen atoms. Representative examples include, but are not limited to, —OCF₃, —OCHF₂, —OCH₂F, and —OCF₂CF₃. Suitably, a haloalkoxy group is selected from —OCHF₂ and —OCF₃, suitably —OCF₃.

The term “heterocyclyl”, “heterocyclic” or “heterocycle” means a non-aromatic saturated or partially saturated monocyclic, fused, bridged, or spiro bicyclic heterocyclic ring system(s). Monocyclic heterocyclic rings contain from about 3 to 12 (suitably from 3 to 7) ring atoms, with from 1 to 5 (suitably 1, 2 or 3) heteroatoms selected from nitrogen, oxygen or sulfur in the ring. Bicyclic heterocycles contain from 7 to 17 member atoms, suitably 7 to 12 member atoms, in the ring. Bicyclic heterocyclic(s) rings may be fused, spiro, or bridged ring systems. Examples of heterocyclic groups include cyclic ethers such as, but not limited to, oxiranyl, oxetanyl, tetrahydrofuranyl, dioxanyl, and substituted cyclic ethers. Heterocycles containing nitrogen include, for example, azetidinyl, pyrrolidinyl, piperidinyl, piperazinyl, tetrahydrotriazinyl, tetrahydropyrazolyl, and the like. Typical sulfur containing heterocycles include tetrahydrothienyl, dihydro-1,3-dithiol, tetrahydro-2H-thiopyran, and hexahydrothiepine. Other heterocycles include dihydrooxathiolyl, tetrahydrooxazolyl, tetrahydro-oxadiazolyl, tetrahydrodioxazolyl, tetrahydrooxathiazolyl, hexahydrotriazinyl, tetrahydrooxazinyl, morpholinyl, thiomorpholinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothienyl and thiomorpholinyl such as, but not limited to, tetrahydrothiene 1,1-dioxide and thiomorpholinyl 1,1-dioxide. A suitable value for a heterocyclyl group which bears 1 or 2 oxo (═O) or thioxo (═S) substituents is, for example, 2-oxopyrrolidinyl, 2-thioxopyrrolidinyl, 2-oxoimidazolidinyl, 2-thioxoimidazolidinyl, 2-oxopiperidinyl, 2,5-dioxopyrrolidinyl, 2,5-dioxoimidazolidinyl or 2,6-dioxopiperidinyl. Particular heterocyclyl groups are saturated monocyclic 3 to 7 membered heterocyclyls containing 1, 2 or 3 heteroatoms selected from nitrogen, oxygen or sulfur, for example azetidinyl, tetrahydrofuranyl, tetrahydropyranyl, pyrrolidinyl, morpholinyl, tetrahydrothienyl, tetrahydrothienyl 1,1-dioxide, thiomorpholinyl, thiomorpholinyl 1,1-dioxide, piperidinyl, homopiperidinyl, piperazinyl or homopiperazinyl. As the skilled person would appreciate, any heterocycle may be linked to another group via any suitable atom, such as via a carbon or nitrogen atom. However, reference herein to piperidino or morpholino refers to a piperidin-1-yl or morpholin-4-yl ring that is linked via the ring nitrogen.

By “bridged ring systems” is meant ring systems in which two rings share more than two atoms, see for example Advanced Organic Chemistry, by Jerry March 4^{th} Edition, Wiley Interscience, pages 131-133, 1992. Examples of bridged heterocyclyl ring systems include, aza-bicyclo[2.2.1]heptane, 2-oxa-5-azabicyclo[2.2.1]heptane, aza-bicyclo[2.2.2]octane, aza-bicyclo[3.2.1]octane and quinuclidine.

By “spiro bicyclic ring systems” we mean that the two ring systems share one common spiro carbon atom, i.e. the heterocyclic ring is linked to a further carbocyclic or heterocyclic ring through a single common spiro carbon atom. Examples of spiro ring systems include 6-azaspiro[3.4]octane, 2-oxa-6-azaspiro[3.4]octane, 2-azaspiro[3.3]heptanes, 2-oxa-6-azaspiro[3.3]heptanes, 7-oxa-2-azaspiro[3.5]nonane, 6-oxa-2-azaspiro[3.4]octane, 2-oxa-7-azaspiro[3.5]nonane and 2-oxa-6-azaspiro[3.5]nonane.

The term “heteroaryl” or “heteroaromatic” means an aromatic mono-, bi-, or polycyclic ring incorporating one or more (for example 14, particularly 1, 2 or 3) heteroatoms selected from nitrogen, oxygen or sulfur. The term heteroaryl includes both monovalent species and divalent species. Examples of heteroaryl groups are monocyclic and bicyclic groups containing from five to twelve ring members, and more usually from five to ten ring members. The heteroaryl group can be, for example, a 5- or 6-membered monocyclic ring or a 9- or 10-membered bicyclic ring, for example a bicyclic structure formed from fused five and six membered rings or two fused six membered rings. Each ring may contain up to about four heteroatoms typically selected from nitrogen, sulfur and oxygen. Typically, the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general, the number of basic nitrogen atoms present in the heteroaryl group, including any amino group substituents of the ring, will be less than five.

Examples of heteroaryl include furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, isoindolyl, benzothienyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl, pteridinyl, naphthyridinyl, carbazolyl, phenazinyl, benzisoquinolinyl, pyridopyrazinyl, thieno[2,3b]-furanyl-, 2H-furo[3,2b]-pyranyl-, 5H-pyrido[2,3-d]-ooxazinyl-, 1H-pyrazolo[4,3-d]-oxazolyl, 4H-imidazo[4,5d]thiazolyl, pyrazino[2,3d]pyridazinyl, -imidazo[2, 1b]thiazolyl, -imidazo[1,2b][1,2,4]-triazinyl. “Heteroaryl” also covers partially aromatic bi- or polycyclic ring systems wherein at least one ring is an aromatic ring and one or more of the other ring(s) is a nonaromatic, saturated or partially saturated ring, provided at least one ring contains one or more heteroatoms selected from nitrogen, oxygen or -sulfur-. Examples of partially aromatic heteroaryl groups include for example, tetrahydroisoquinolinyl, tetrahydroquinolinyl, 2-oxo-1,2,3,4-tetrahydroquinolinyl, dihydrobenzthienyl, dihydrobenzfuranyl, 2,3-dihydro-benzo[1,4]dioxinyl, benzo[1,3]dioxolyl, 2,2-dioxo-1,3-dihydro-2-benzothienyl, 4,5,6,7-tetrahydrobenzofuranyl, indolinyl, 1,2,3,4-tetrahydro-1,8-naphthyridinyl, 1,2,3,4-tetrahydropyrido[2,3-b]pyrazinyl, 3,4-dihydro-2H-pyrido[3,2-b][1,4]oxazinyl and 6,8-dihydro-5H-[1,2,4]triazolo[4,3-a]pyrazinyl.

Examples of five membered heteroaryl groups include but are not limited to pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl and tetrazolyl groups.

Examples of six membered heteroaryl groups include but are not limited to pyridyl, pyrazinyl, pyridazinyl, pyrimidinyl and triazinyl.

A bicyclic heteroaryl group may be, for example, a group selected from:
a benzene ring fused to a 5- or 6-membered ring containing 1, 2 or 3 ring heteroatoms;
a pyridine ring fused to a 5- or 6-membered ring containing 1, 2 or 3 ring heteroatoms;
a pyrimidine ring fused to a 5- or 6-membered ring containing 1 or 2 ring heteroatoms;
a pyrrole ring fused to a 5- or 6-membered ring containing 1, 2 or 3 ring heteroatoms;
a pyrazole ring fused to a 5- or 6-membered ring containing 1 or 2 ring heteroatoms;
a pyrazine ring fused to a 5- or 6-membered ring containing 1 or 2 ring heteroatoms;
an imidazole ring fused to a 5- or 6-membered ring containing 1 or 2 ring heteroatoms;
an oxazole ring fused to a 5- or 6-membered ring containing 1 or 2 ring heteroatoms;
an isoxazole ring fused to a 5- or 6-membered ring containing 1 or 2 ring heteroatoms;
a thiazole ring fused to a 5- or 6-membered ring containing 1 or 2 ring heteroatoms;
an isothiazole ring fused to a 5- or 6-membered ring containing 1 or 2 ring heteroatoms;
a thiophene ring fused to a 5- or 6-membered ring containing 1, 2 or 3 ring heteroatoms;
a furan ring fused to a 5- or 6-membered ring containing 1, 2 or 3 ring heteroatoms;
a cyclohexyl ring fused to a 5- or 6-membered heteroaromatic ring containing 1, 2 or 3 ring heteroatoms; and
a cyclopentyl ring fused to a 5- or 6-membered heteroaromatic ring containing 1, 2 or 3 ring heteroatoms.

Particular examples of bicyclic heteroaryl groups containing a six membered ring fused to a five membered ring include but are not limited to benzfuranyl, benzthiophenyl, benzimidazolyl, benzoxazolyl, benzisoxazolyl, benzthiazolyl, benzisothiazolyl, isobenzofuranyl, indolyl, isoindolyl, indolizinyl, indolinyl, isoindolinyl, purinyl (e.g., adeninyl, guaninyl), indazolyl, benzodioxolyl and pyrazolopyridinyl groups.

Particular examples of bicyclic heteroaryl groups containing two fused six membered rings include but are not limited to quinolinyl, isoquinolinyl, chromanyl, thiochromanyl, chromenyl, isochromenyl, chromanyl, isochromanyl, benzodioxanyl, quinolizinyl, benzoxazinyl, benzodiazinyl, pyridopyridinyl, quinoxalinyl, quinazolinyl, cinnolinyl, phthalazinyl, naphthyridinyl and pteridinyl groups.

The term “aryl” means a cyclic or polycyclic aromatic ring having from 5 to 12 carbon atoms. The term aryl includes both monovalent species and divalent species. Examples of aryl groups include, but are not limited to, phenyl, biphenyl, naphthyl and the like. In particular embodiment, an aryl is phenyl.

This specification also makes use of several composite terms to describe groups comprising more than one functionality. Such terms will be understood by a person skilled in the art. For Example, heterocyclyl(m-nC)alkyl comprises (m-nC)alkyl substituted by heterocyclyl.

The term “aryl(1-2C)alkyl” means an aryl group covalently attached to a (1-2C)alkylene group, both of which are defined herein. Examples of aryl-(1-2C)alkyl groups include benzyl, phenylethyl, and the like.

“Heteroaryl(1-3C)alkyl” means a heteroaryl group covalently attached to a (1-3C)alkylene group, both of which are defined herein. Examples of heteroaryl-alkyl groups include pyridin-3-ylmethyl, 2-(benzofuran-2-yl)ethyl, and the like.

“Heterocyclyl(1-2C)alkyl” means a heterocyclyl group covalently attached to a (1-2C)alkylene group, both of which are defined herein.

“(3-6C)cycloalkyl-(1-2C)alkyl” means a (3-6C)cycloalkyl group covalently attached to a (1-2C)alkylene group, both of which are defined herein.

The term “optionally substituted” refers to either groups, structures, or molecules that are substituted and those that are not substituted. The term “wherein a/any CH, CH₂, CH₃ group or heteroatom (i.e. NH) within a R¹ group is optionally substituted” suitably means that (any) one of the hydrogen radicals of the R¹ group is substituted by a relevant stipulated group.

Where optional substituents are chosen from “one or more” groups it is to be understood that this definition includes all substituents being chosen from one of the specified groups or the substituents being chosen from two or more of the specified groups.

A wavy bond ( [symbol] ) is used herein to show a point of attachment.

The phrase “compound of the invention” means those compounds which are disclosed herein, both generically and specifically.

As used herein by itself or in conjunction with another term or terms, “pharmaceutically acceptable” refers to materials that are generally chemically and/or physically compatible with other ingredients (such as, for example, with reference to a formulation), and/or are generally physiologically compatible with the recipient (such as, for example, a subject) thereof.

As used herein by themselves or in conjunction with another term or terms, “subject(s)” and “patient(s)”, suitably refer to mammals, in particular humans.

### Compounds of the Invention

In a first aspect, the present invention relates to a compound, or pharmaceutically acceptable salt, hydrate or solvate thereof, having the structural formula I shown below:

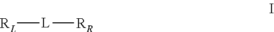

wherein:
L is linker that separates R_{L} from R_{R} by 7 to 13, 8 to 12, 9 to 11, or 8, 9 or 10 bond lengths, with the proviso that L is not a group of the formula:

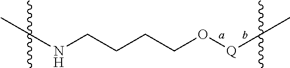

wherein Q is selected from formula Ia or Ib shown below:

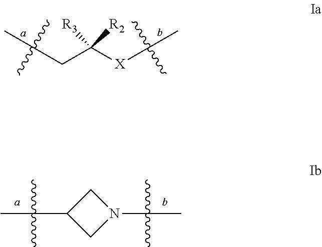

wherein:
bond a in formulae Ia and Ib corresponds with bond a in the above formula and
bond b in formulae Ia and Ib corresponds with bond b in the above formula;
R₂ and R₃ are each independently selected from hydrogen or methyl; and
X is NH or O;
R_{L} is selected from one of formulae Ic, Id, Ie, If, Ig, Ih, Ii, Ij, Ik or Il shown below:

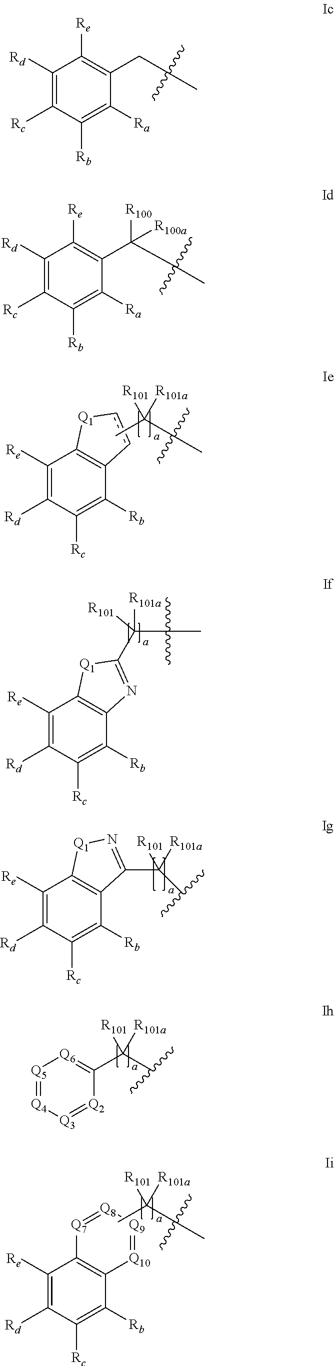

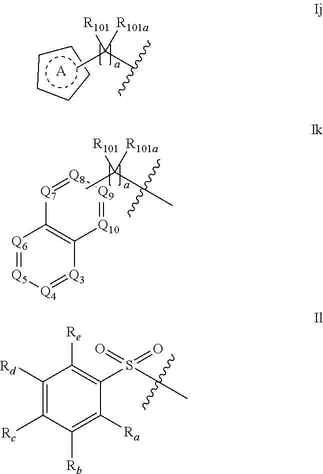

wherein:
Rₐ and R_{b} are each independently selected from hydrogen, methyl or halo;
R_{b} and R_{d} are each independently selected from hydrogen, halo, cyano, (1-4C)alkyl, (3-6C)cycloalkyl,
   —[CH₂]₀₋₃-(1-4C)alkoxy,
   —[CH₂]₀₋₃—C(O)NH₂,
   —[CH₂]₀₋₃—C(O)NH(1-4C)alkyl,
   —[CH₂]₀₋₃—C(O)N[(1-4C)alkyl]₂,
   —[CH₂]₀₋₃—NH₂,
   —[CH₂]₀₋₃—NH(1-4C)alkyl,
   —[CH₂]₀₋₃—N[(1-4C)alky]₂,
   —[CH₂]₀₋₃—S(O)—(1-4C)alkyl (wherein q is 0, 1 or 2),
   —[CH₂]₀₋₃—C(O)(1-4C)alkyl,
   —[CH₂]₀₋₃—C(O)OH,
   —[CH₂]₀₋₃—C(O)O-(1-4C)alkyl,
   —[CH₂]₀₋₃—N(R_{f})C(O)-(1-4C)alkyl (wherein R_{f} is hydrogen or methyl),
   —[CH₂]₀₋₃—S(O)₂NH(1-4C)alkyl,
   —[CH₂]₀₋₃—S(O)₂N[(1-4C)alkyl]₂,
   —[CH₂]₀₋₃—N(R_{g})SO₂-(1-4C)alkyl (wherein R_{g} is hydrogen or methyl),
   a group of the formula:

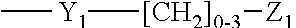

wherein Y₁ is absent, —O—, —NH—, —NMe-, —S—, —S(O)— or —S(O)₂—; and
Z₁ is (3-6C)cycloalkyl, phenyl, a 4- to 6-membered heterocyclyl or 5 or 6-membered heteroaryl;
and wherein:
any alkyl, alkoxy, cycloalkyl or —[CH₂] moiety within a R_{b} and R_{d} substituent group is optionally substituted by one or more substituents selected from halo, hydroxy, cyano, amino, —C(O)OH, —C(O)NH₂, (1-2C)alkoxy, or (3-4C)cycloalkoxy; and
Z₁ is optionally substituted by one or more substituents selected from: halo, hydroxy, cyano, amino, —C(O)OH, —C(O)NH₂, (1-2C)alkoxy, (1-2C)alkyl, (3-4C)cycloalkyl, (3-4C)cycloalkoxy, —C(O)NH(1-2C)alkyl, —C(O)N[(1-2C)alkyl]₂, —NH(1-2C)alkyl, —N[(1-2C)alkyl]₂, —S(O)_{q}-(1-2C)alkyl (wherein q is 0, 1 or 2), —C(O)(1-2C)alkyl, —C(O)O-(1-2C)alkyl, —N(R_{f})C(O)-(1-2C)alkyl, —S(O)₂NH(1-2C)alkyl, —S(O)₂N[(1-2C)alkyl]₂, or —NHSO₂-(1-2C)alkyl, and wherein any (1-2C)alkoxy, (1-2C)alkyl, (3-4C)cycloalkyl or (3-4C)cycloalkoxy group is optionally substituted by one or more substituents selected from halo, cyano, hydroxy, (1-2C)alkyl, (1-2C)alkoxy or (1-2C)alkoxy-(1-2C)alkyl;
R_{c} is selected from hydrogen, halo, cyano, —C(O)NH₂, (1-4C)alkyl,
   —[CH₂]₀₋₃-(1-4C)alkoxy,
   —[CH₂]₀₋₃-(3-6C)cycloalkoxy,
   —[CH₂]₀₋₃—C(O)NH₂,
   —[CH₂]₀₋₃—C(O)NH(1-4C)alkyl,
   —[CH₂]₀₋₃—C(O)N[(1-4C)alkyl]₂,
   —[CH₂]₀₋₃—NH₂,
   —[CH₂]₀₋₃—NH(1-4C)alkyl,
   —[CH₂]₀₋₃—N[(1-4C)alkyl]₂,
   —[CH₂]₀₋₃—S(O)_{q}-(1-4C)alkyl (wherein q is 0, 1 or 2),
   —[CH₂]₀₋₃—C(O)(1-4C)alkyl,
   —[CH₂]₀₋₃—C(O)OH,
   —[CH₂]₀₋₃—C(O)O-(1-4C)alkyl,
   —[CH₂]₀₋₃—N(Rₕ)C(O)-(1-4C)alkyl (wherein Rₕ is hydrogen or methyl),
   —[CH₂]₀₋₃—S(O)₂NH(1-4C)alkyl,
   —[CH₂]₀₋₃—S(O)₂N[(1-4C)alkyl]₂,
   —[CH₂]₀₋₃—N(Rᵢ)SO₂-(1-4C)alkyl (wherein Rᵢ is hydrogen or methyl),
   a group of the formula:

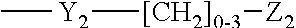

wherein Y₂ is absent, —O—, —NH—, —NMe-, —S—, —S(O)— or —S(O)₂—; and
Z₂ is (3-6C)cycloalkyl, phenyl, a 4- to 6-membered heterocyclyl or 5 or 6-membered heteroaryl;
and wherein:
   any alkyl, alkoxy, cycloalkyl or —[CH₂]— moiety within a R_{c} substituent group is optionally substituted by one or more substituents selected from halo, hydroxy, cyano, amino, —C(O)OH, —C(O)NH₂, (1-2C)alkoxy, or (3-4C)cycloalkoxy; and
   Z₂ is optionally substituted by one or more substituents selected from: halo, hydroxy, cyano, amino, —C(O)OH, —C(O)NH₂, (1-2C)alkoxy, (1-2C)alkyl, (3-4C)cycloalkyl, (3-4C)cycloalkoxy, —C(O)NH(1-2C)alkyl, —C(O)N[(1-2C)alkyl]₂, —NH(1-2C)alkyl, —N[(1-2C)alkyl]₂, —S(O)_{q}-(1-2C)alkyl (wherein q is 0, 1 or 2), C(O)(1-2C)alkyl, —C(O)O-(1-2C)alkyl, —N(R_{f})C(O)-(1-2C)alkyl, —S(O)₂NH(1-2C)alkyl, —S(O)₂N[(1-2C)alkyl]₂, or —NHSO₂-(1-2C)alkyl, and wherein any (1-2C)alkoxy, (1-2C)alkyl, (3-4C)cycloalkyl or (3-4C)cycloalkoxy group is optionally substituted by one or more substituents selected from halo, cyano, hydroxy, (1-2C)alkyl, (1-2C)alkoxy or (1-2C)alkoxy-(1-2C)alkyl;
R₁₀₀ is (1-2C)alkyl optionally substituted by hydroxy or halo;
R₁₀₀ₐ is selected from hydrogen or methyl;
or R₁₀₀ and R₁₀₀ₐ are linked to form a cyclopropyl or cyclobutyl ring;
R₁₀₁ is hydrogen or (1-2C)alkyl optionally substituted by hydroxy or halo;
R₁₀₁ₐ is selected from hydrogen or methyl;
or R₁₀₁ and R₁₀₁ₐ are linked to form a cyclopropyl or cyclobutyl ring;
integer a is 0, 1 or 2;
Q₁ is selected from —NR₁₀₂—, —O—, —S— or —CH—;
R₁₀₂ is hydrogen or (1-2C)alkyl;
[symbol] represents a single or double bond;
Q₂ is N or CRₐ;
Q₃ is N or CR_{b};
Q₄ is N or CR_{c};
Q₅ is N or CR_{d};
Q₆ is N or CRₑ;
Rₐ, R_{b}, R_{c}, R_{d} and Rₑ are each as defined above;
   with the proviso that one to three of Q₂, Q₃, Q₄, Q₅ or Q₆ is/are N;
Q₇ is N or CR_{f};
Q₈ is N or CR_{f};
Q₉ is N or CR_{f};
Q₁₀ is N or CR_{f};
   with the proviso that one or two of Q₇, Q₈, Q₉ or Q₁₀ is/are N;
each R_{f} present is independently selected from hydrogen, methyl or halo;
Ring A is a five-membered heteroaryl ring optionally substituted by one R_{b} and/or one or two R_{c} substituents;
   R_{R} is selected from one of formulae Im, In, Io, Ip, Iq or Ir shown below:

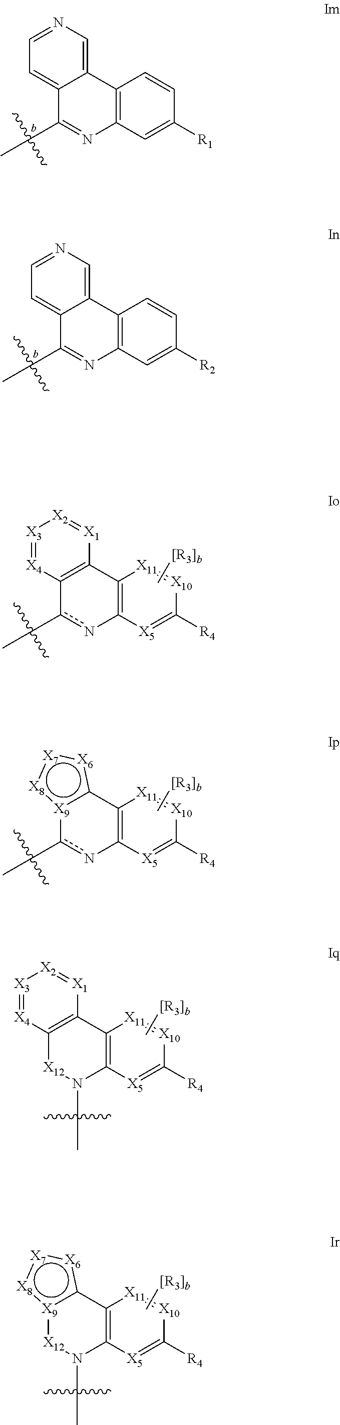

wherein:
[symbol] denotes the point of attachment;
[symbol] denotes an optional double bond;
R₁ is selected from —C(O)OH or —C(O)NH₂;
R₂ is selected from:
   (i) —C(O)NHR₂ₐ or —C(O)OR₂ₐ, wherein R₂ₐ is (1-4C)alkyl optionally substituted by halo, hydroxy, amino or carboxyl;
   (ii) —N(R_{2b})C(O)R_{2c}, wherein R_{2b} is hydrogen or methyl and R_{2c} is (1-4C)alkyl optionally substituted by halo, hydroxy, amino or carboxyl;
   (iii) —CN;
   (iv) a 5- or 6-membered heteroaryl comprising one, two, three or four nitrogen atoms;
   (v) oxetanyl, tetrahydrofuranyl or tetrahydropyranyl;
R₃ is selected from methyl, fluoro or chloro and integer b is 0 or 1;
R₄ is a group R₁ or R₂ as defined above;
X₁ is CH, CRₓ₁ or N;
X₂ is CH, CRₓ₂ or N;
X₃ is CH, CRₓ₃ or N;
X₄ is CH, CRₓ₄ or N;
X₅/X₁₀/X₁₁ are each independently CH or N;
with the proviso that:
   if X₂ is N and R₄ is a R₁ group, then either at least one of X₁, X₃ or X₄ is N or CRₓ₁, CRₓ₃, CRₓ₄ respectively, and/or X₅/X₁₀/X₁₁ is N, and/or integer b is 1;
Rₓ₁, Rₓ₂, Rₓ₃ and Rₓ₄ are each independently selected from methyl, hydroxy, fluoro, chloro or NR₅₀R₅₁, wherein R₅₀ and R₅₁ are each independently selected from hydrogen, (1-2C)alkyl or (3-4C)cycloalkyl;
X₆ is CH, CRₓ₅, O, S, NH, NMe or N;
X₇ is CH, CRₓ₆, O, S, NH, NMe or N;
X₈ is CH, CRₓ₇, O, S, NH, NMe or N;
X₉ is C or N;
X₁₂ is CH or C(O);
with the proviso that only one of X₆, X₇ or X₈ can be O, S, NH or NMe;
and Rₓ₅, Rₓ₆ and Rₓ₇ are each independently selected from methyl, hydroxy, fluoro, chloro or NR₅₂R₅₃, wherein R₅₂ and R₅₃ are each independently selected from hydrogen, (1-2C)alkyl or (3-4C)cycloalkyl.

The provisos described in relation to the first aspect of the invention will be understood to be applicable to all embodiments of the compounds of the invention described herein, where relevant.

Suitably, in the compounds of formula I:
(i) only one or two of X₅/X₁₀/X₁₁ can be N; and
(ii) one to five of X₁, X₂, X₃, X₄ or X₅/X₁₀/X₁₁ is/are N.

Particular compounds of the invention include, for example, compounds of the formula I, or pharmaceutically acceptable salts, hydrates and/or solvates thereof, wherein, unless otherwise stated, each of R_{L}, L and R_{R} each have any of the meanings defined hereinbefore or are as defined in any one of paragraphs (1) to (108) hereinafter:—
(1) L is linker that separates R_{L} from R_{R} by 7 to 13, 8 to 12, or 8, 9 or 10 bond lengths, with the proviso that L is not a group of the formula:

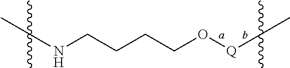

wherein Q is selected from formula Ia or Ib shown below:

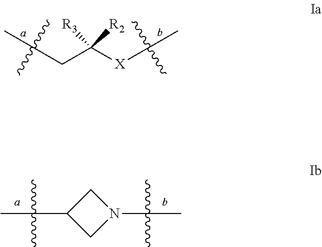

wherein:
bond a in formulae Ia and Ib corresponds with bond a in the above formula and
bond b in formulae Ia and Ib corresponds with bond b in the above formula;
R₂ and R₃ are each hydrogen; and
X is NH or O;
(2) L is linker that separates R_{L} from R_{R} by 8 to 12, or 10 bond lengths, with the proviso that L is not a group of the formula:

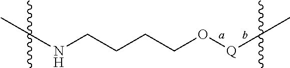

wherein Q is selected from formula Ia or Ib shown below:

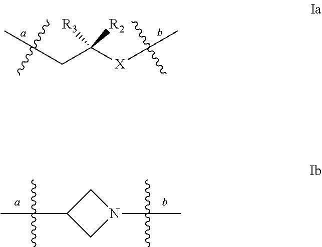

wherein:
bond a in formulae Ia and Ib corresponds with bond a in the above formula and
bond b in formulae Ia and Ib corresponds with bond b in the above formula;
R₂ and R₃ are each hydrogen; and
X is NH or O;
(3) L is linker that separates R_{L} from R_{R} by 8, 9, or 10 bond lengths, with the proviso that L is not a group of the formula:

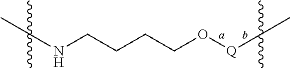

wherein Q is selected from formula Ia or Ib shown below:

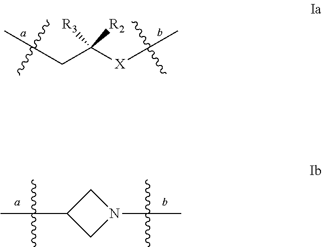

wherein:
bond a in formulae Ia and Ib corresponds with bond a in the above formula and
bond b in formulae Ia and Ib corresponds with bond b in the above formula;
R₂ and R₃ are each hydrogen; and
X is NH or O;
(4) L is linker [that suitably separates R_{L} from R_{R} by 7 to 13, 8 to 12, or 8, 9 or 10 bond lengths] selected from:
   (i) a group of the formula

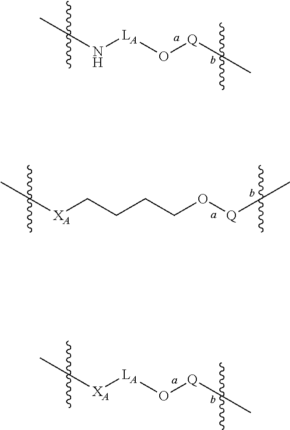

wherein LA is selected from:
   (i) a (4C)alkylene substituted by one or more R_{LA} substituent groups;
   (ii) a (3C)alkylene or a (5C)alkylene optionally substituted by one or more R_{LA} substituent groups;
   (iii) a (0-3C)alkylene-(3-6C)cycloalkylene-(0-3C)alkylene-group optionally substituted by one or more R_{LA} substituent groups;
each R_{LA} group present is selected from hydroxy, halo, (1-2C)alkyl, (1-2C) hydroxyalkyl or (1-2C)haloalkyl;
X_{A} is selected from: —O—, —S—, —SO—, —SO₂—, —N(Me)-, —C(O)—, —C(O)O—, —OC(O)—, —C(O)N(Rₓₐ)—, —N(Rₓₐ)C(O)—, —N(R_{xb})C(O)N(Rₓₐ)—, —S(O)₂N(Rₓₐ)— or —N(Rₓₐ)SO₂—, wherein Rₓₐ and R_{xb} are selected from hydrogen or methyl;
Q is selected from formula Ia or Ib shown below:

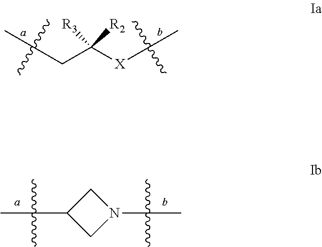

wherein:
bond a in formulae Ia and Ib corresponds with bond a in the above formula and bond b in formulae Ia and Ib corresponds with bond b in the above formula;
R₂ and R₃ are each independently selected from hydrogen or methyl; and
X is NH or O;
(ii) a group of the formula:

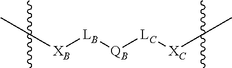

wherein:
X_{B} is selected from: —O—, —S—, —SO—, —SO₂—, —N(Rₓₐ)—, —C(O)—, —C(O)O—, —OC(O)—, —C(O)N(Rₓₐ)—, —N(Rₓₐ)C(O)—, —N(R_{xb})C(O)N(Rₓₐ)—, —S(O)₂N(Rₓₐ)— or —N(Rₓₐ)SO₂—, wherein Rₓₐ and R_{xb} are selected from hydrogen or methyl;
L_{B} is absent or selected from:
(i) a (1-5C)alkylene optionally substituted by one or more R_{LA} substituent groups;
(ii) a (0-3C)alkylene-(3-6C)cycloalkylene-(0-3C)alkylene-group optionally substituted by one or more R_{LA} substituent groups;
Q_{B} is selected from a 5 or 6-membered heteroaryl ring, a 9 or 10-membered bicyclic heteroaryl ring, a phenyl, or a 4 to 6 membered heterocyclic ring;
L_{C} is absent or selected from:
(i) a (1-5C)alkylene optionally substituted by one or more R_{LA} substituent groups;
(ii) a (0-3C)alkylene-(3-6C)cycloalkylene-(0-3C)alkylene-group optionally substituted by one or more R_{LA} substituent groups; and
X_{C} is selected from: —O—, —S—, —SO—, —SO₂—, —N(Rₓₐ)—, —C(O)—, —C(O)O—, —OC(O)—, —C(O)N(Rₓₐ)—, —N(Rₓₐ)C(O)—, —N(R_{xb})C(O)N(Rₓₐ)—, —S(O)₂N(Rₓₐ)— or —N(Rₓₐ)SO₂—, wherein Rₓₐ and R_{xb} are selected from hydrogen or methyl;
(iii) a group of the formula:

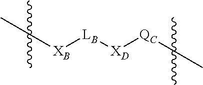

wherein:
X_{B} and L_{B} are each as defined above;
X_{D} is absent or selected from: —O—, —S—, —SO—, —SO₂—, —N(Rₓₐ)—, —C(O)—, —C(O)O—, —OC(O)—, —C(O)N(Rₓₐ)—, —N(Rₓₐ)C(O)—, —N(R_{xb})C(O)N(Rₓₐ)—, —S(O)₂N(Rₓₐ)— or —N(Rₓₐ)SO₂—, wherein Rₓₐ and R_{xb} are selected from hydrogen or methyl;
Q_{C} is selected a 5 to 7 membered heterocyclic ring;
(iv) a group of the formula:

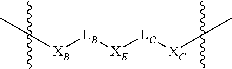

wherein:
X_{B}, L_{B}, L_{C} and X_{C} are each as defined above;
X_{E} is selected from: —O—, —S—, —SO—, —SO₂—, —N(Rₓₐ)—, —C(O)—, —C(O)O—, —OC(O)—, —C(O)N(Rₓₐ)—, —N(Rₓₐ)C(O)—, —N(R_{xb})C(O)N(Rₓₐ)—, —S(O)₂N(Rₓₐ)— or —N(Rₓₐ) SO₂—, wherein Rₓₐ and R_{xb} are selected from hydrogen or methyl;
wherein X_{E} is not —O— when X_{B} is —NH—, L_{B} is butylene, L_{C} is ethylene, and X_{C} is —NH— or —O—;
(v) a group of the formula:

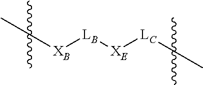

wherein:
X_{B}, L_{B}, L_{C} and X_{E} are each as defined above;
(vi) a group of the formula:

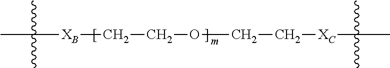

wherein:
X_{B} and X_{C} are each as defined above;
integer m is 1 or 2;
(5) L is linker [that suitably separates R_{L} from R_{R} by 7 to 13, 8 to 12, or 8, 9 or 10 bond lengths] selected from:
   (i) a group of the formula

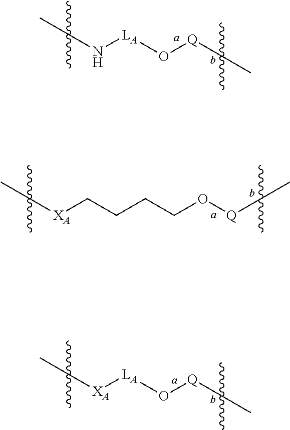

wherein L_{A} is selected from:
   (i) a (4C)alkylene substituted by one or more R_{LA} substituent groups;
   (ii) a (3C)alkylene or a (5C)alkylene optionally substituted by one or more R_{LA} substituent groups;
   (iii) a (0-2C)alkylene-(3-6C)cycloalkylene-(1-3C)alkylene-group optionally substituted by one or more R_{LA} substituent groups;
each R_{LA} group present is selected from hydroxy, halo, (1-2C)alkyl, (1C) hydroxyalkyl or (1C)haloalkyl;
X_{A} is selected from: —O—, —S—, —SO—, —SO₂—, —N(Me)-, —N(Rₓₐ)C(O)—, —N(R_{xb})C(O)N(Rₓₐ)—, —S(O)₂N(Rₓₐ)— or —N(Rₓₐ)SO₂—, wherein Rₓₐ and R_{xb} are selected from hydrogen or methyl;
Q is selected from formula Ia or Ib shown below:

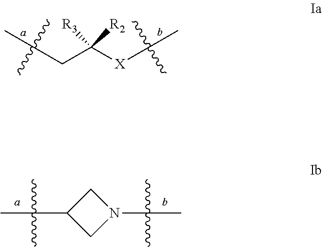

wherein:
bond a in formulae Ia and Ib corresponds with bond a in the above formula and bond b in formulae Ia and Ib corresponds with bond b in the above formula;
R₂ and R₃ are each independently selected from hydrogen or methyl; and
X is NH or O;
(ii) a group of the formula:

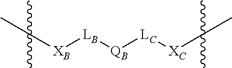

wherein:
X_{B} is selected from: —O—, —S—, —SO—, —SO₂—, —N(Rₓₐ)—, —N(Rₓₐ)C(O)—, —N(R_{xb})C(O)N(Rₓₐ)—, —S(O)₂N(Rₓₐ)— or —N(Rₓₐ)SO₂—, wherein Rₓₐ and R_{xb} are selected from hydrogen or methyl;
L_{B} is absent or selected from:
(i) a (1-3C)alkylene optionally substituted by one or more R_{LA} substituent groups;
(ii) a (0-2C)alkylene-(3-6C)cycloalkylene-(1-3C)alkylene-group optionally substituted by one or more R_{LA} substituent groups;
Q_{B} is selected from a 5 or 6-membered heteroaryl ring, a 9 or 10-membered bicyclic heteroaryl ring, a phenyl, or a 4 to 6 membered heterocyclic ring;
L_{C} is absent or selected from:
(i) a (1-5C)alkylene optionally substituted by one or more R_{LA} substituent groups;
(ii) a (0-2C)alkylene-(3-6C)cycloalkylene-(1-3C)alkylene-group optionally substituted by one or more R_{LA} substituent groups; and
X_{C} is selected from: —O—, —N(Rₓₐ)—, —C(O)—, —C(O)O—, —OC(O)—, —C(O)N(Rₓₐ)—, —N(Rₓₐ)C(O)—, —N(R_{xb})C(O)N(Rₓₐ)—, —S(O)₂N(Rₓₐ)— or —N(Rₓₐ)SO₂—, wherein Rₓₐ and R_{xb} are selected from hydrogen or methyl;
(iii) a group of the formula:

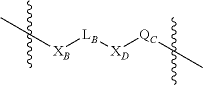

wherein:
X_{B} and L_{B} are each as defined above;
X_{D} is absent or selected from: —O—, —N(Rₓₐ)—, —C(O)—, —C(O)O—, —OC(O)—, —C(O)N(Rₓₐ)—, —N(Rₓₐ)C(O)—, —N(R_{xb})C(O)N(Rₓₐ)—, —S(O)₂N(Rₓₐ)— or —N(Rₓₐ)SO₂—, wherein Rₓₐ and R_{xb} are selected from hydrogen or methyl;
Q_{C} is selected a 5 to 7 membered heterocyclic ring;
(iv) a group of the formula:

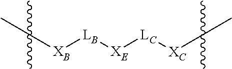

wherein:
X_{B}, L_{B}, L_{C} and X_{C} are each as defined above;
X_{E} is selected from: —O—, —N(Rₓₐ)—, —C(O)—, —C(O)O—, —OC(O)—, —C(O)N(Rₓₐ)—, —N(Rₓₐ)C(O)—, —N(R_{xb})C(O)N(Rₓₐ)—, —S(O)₂N(Rₓₐ)— or —N(Rₓₐ)SO₂—, wherein Rₓₐ and R_{xb} are selected from hydrogen or methyl;
wherein X_{E} is not —O— when X_{B} is —NH—, L_{B} is butylene, L_{C} is ethylene, and X_{C} is —NH— or —O—;
(v) a group of the formula:

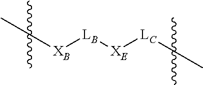

wherein:
X_{B}, L_{B}, L_{C} and X_{E} are each as defined above;
(vi) a group of the formula:

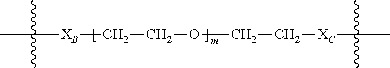

wherein:
X_{B} and X_{C} are each as defined above;
integer m is 2;
(6) L is linker [that suitably separates R_{L} from R_{R} by 7 to 13, 8 to 12, or 8, 9 or 10 bond lengths] selected from:
   (i) a group of the formula

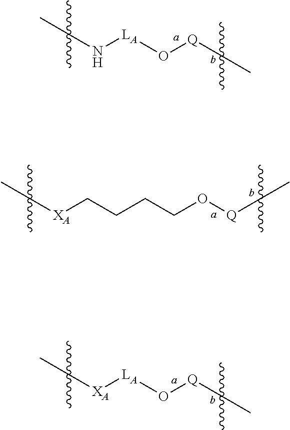

wherein L_{A} is selected from:
   (i) a (4C)alkylene substituted by one or more R_{LA} substituent groups;
   (ii) a (3C)alkylene or a (5C)alkylene optionally substituted by one or more R_{LA} Substituent groups;
   (iii) a (0-1C)alkylene-(3-6C)cycloalkylene-(1-3C)alkylene-group optionally substituted by one or more R_{LA} substituent groups;
each R_{LA} group present is selected from hydroxy, halo or (1-2C)alkyl;
X_{A} is selected from: —O—, —S—, —SO—, —SO₂—, —N(Me)-, —S(O)₂N(Rₓₐ)— or —N(Rₓₐ)SO₂—, wherein Rₓₐ and R_{xb} are selected from hydrogen or methyl;
Q is selected from formula Ia or Ib shown below:

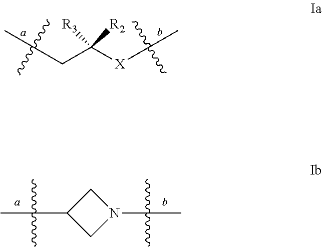

wherein:
bond a in formulae Ia and Ib corresponds with bond a in the above formula and bond b in formulae Ia and Ib corresponds with bond b in the above formula;
R₂ and R₃ are both hydrogen; and
X is NH or O;
(ii) a group of the formula:

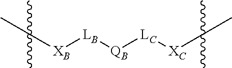

wherein:
X_{B} is selected from: —O—, —S—, —SO—, —SO₂—, —N(Rₓₐ)—, —S(O)₂N(Rₓₐ)— or —N(Rₓₐ)SO₂—, wherein Rₓₐ and R_{xb} are selected from hydrogen or methyl;
L_{B} is absent or selected from:
(i) a (1-3C)alkylene optionally substituted by one or more R_{LA} substituent groups;
Q_{B} is selected from a 5 or 6-membered heteroaryl ring, a 9 or 10-membered bicyclic heteroaryl ring, a phenyl, or a 4 to 6 membered heterocyclic ring;
L_{C} is absent or selected from:
(i) a (1-5C)alkylene optionally substituted by one or more R_{LA} substituent groups;
X_{C} is selected from: —O—, —N(Rₓₐ)—, —C(O)—, —C(O)O—, —OC(O)—, —C(O)N(Rₓₐ)—, —N(Rₓₐ)C(O)— or —N(R)C(O)N(Rₓₐ)—, wherein Rₓₐ and R_{xb} are selected from hydrogen or methyl;
(iii) a group of the formula:

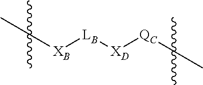

wherein:
X_{B} and L_{B} are each as defined above;
X_{D} is absent or selected from: —O—, —N(Rₓₐ)—, —C(O)—, —C(O)O—, —OC(O)— or —C(O)N(Rₓₐ)— wherein Rₓₐ and R_{xb} are selected from hydrogen or methyl;
Q_{C} is selected a 5 to 7 membered heterocyclic ring;
(iv) a group of the formula:

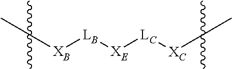

wherein:
X_{B}, L_{B}, L_{C} and X_{C} are each as defined above;
X_{E} is selected from: —O—, —N(Rₓₐ)—, —C(O)—, —C(O)O—, —OC(O)—, —C(O)N(Rₓₐ)—, —N(Rₓₐ)C(O)—, —N(R_{xb})C(O)N(Rₓₐ)—, —S(O)₂N(Rₓₐ)— or —N(Rₓₐ)SO₂—, wherein Rₓₐ and R_{xb} are selected from hydrogen or methyl;
wherein X_{E} is not —O— when X_{B} is —NH—, L_{B} is butylene, L_{C} is ethylene, and X_{C} is —NH— or —O—;
(v) a group of the formula:

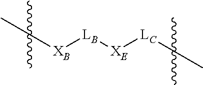

wherein:
X_{B}, L_{B}, L_{C} and X_{E} are each as defined above;
(vi) a group of the formula:

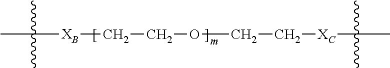

wherein:
X_{B} and X_{C} are each as defined above;
integer m is 2;
(7) L is linker [that suitably separates R_{L} from R_{R} by 7 to 13, 8 to 12, or 8, 9 or 10 bond lengths] selected from:
   (i) a group of the formula

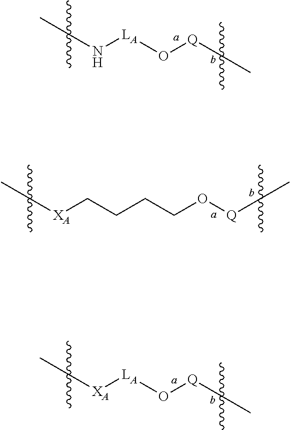

wherein L_{A} is selected from:
   (i) a (4C)alkylene substituted by one or more R_{LA} substituent groups;
   (ii) a (3C)alkylene or a (5C)alkylene optionally substituted by one or more R_{LA} substituent groups;
   (iii) a (3-5C)cycloalkylene-(1-3C)alkylene-group optionally substituted by one or more R_{LA} substituent groups;
each R_{LA} group present is selected from (1-2C)alkyl;
X_{A} is selected from: —N(Me)-, —S(O)₂N(Rₓₐ)— or —N(Rₓₐ) SO₂—, wherein Rx and R_{xb} are selected from hydrogen or methyl;
Q is selected from formula Ia or Ib shown below:

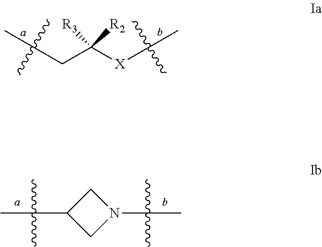

wherein:
bond a in formulae Ia and Ib corresponds with bond a in the above formula and bond b in formulae Ia and Ib corresponds with bond b in the above formula;
R₂ and R₃ are both hydrogen; and
X is NH or O;
(ii) a group of the formula:

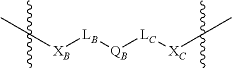

wherein:
X_{B} is selected from: —N(Rₓₐ)— or —S(O)₂N(Rₓₐ)—, wherein Rₓₐ and R_{xb} are selected from hydrogen or methyl;
L_{B} is absent or selected from:
(i) a (1-2C)alkylene optionally substituted by one or more R_{LA} substituent groups;
Q_{B} is selected from a 5-membered heteroaryl ring, a 9-membered bicyclic heteroaryl ring, a phenyl, or a 5 to 6 membered heterocyclic ring;
L_{C} is absent or selected from:
(i) a (2-4C)alkylene optionally substituted by one or more R_{LA} substituent groups;
X_{C} is selected from: —O—, —N(Rₓₐ)—, —C(O)N(Rₓₐ)— or —N(Rₓₐ)C(O)—, wherein Rₓₐ and R_{xb} are selected from hydrogen or methyl;
(iii) a group of the formula:

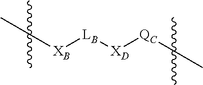

wherein:
X_{B} and L_{B} are each as defined above;
X_{D} is absent or selected from: —O—, —N(Rₓₐ)— or —C(O)N(Rₓₐ)— wherein Rₓₐ and R_{xb} are selected from hydrogen or methyl;
Q_{C} is selected a 5 to 6 membered heterocyclic ring;
(iv) a group of the formula:

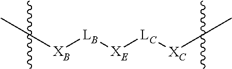

wherein:
X_{B}, L_{B}, L_{C} and X_{C} are each as defined above;
X_{E} is selected from: —O—, —N(Rₓₐ)—, —C(O)N(Rₓₐ)—, —N(Rₓₐ)C(O)—, —N(R_{xb})C(O)N(Rₓₐ)—, —S(O)₂N(Rₓₐ)— or —N(Rₓₐ) SO₂—, wherein Rₓₐ and R_{xb} are selected from hydrogen or methyl;
wherein X_{E} is not —O— when X_{B} is —NH—, L_{B} is butylene, L_{C} is ethylene, and X_{C} is —NH— or —O—;
(v) a group of the formula:

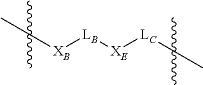

wherein:
X_{B}, L_{B}, L_{C} and X_{E} are each as defined above;
(vi) a group of the formula:

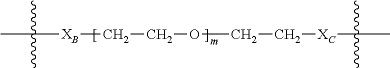

wherein:
X_{B} and X_{C} are each as defined above;
integer m is 2;
(8) L is linker [that suitably separates R_{L} from R_{R} by 7 to 13, 8 to 12, or 8, 9 or 10 bond lengths] selected from:
   (i) a group of the formula

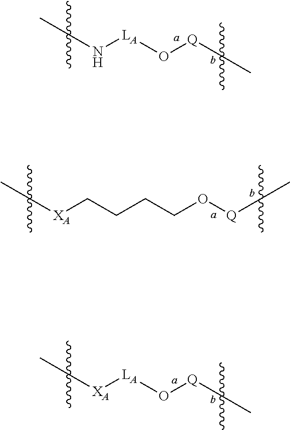

wherein L_{A} is selected from:
   (i) a (4C)alkylene substituted by one R_{LA} substituent groups;
   (ii) a (3C)alkylene or a (5C)alkylene optionally substituted by one or more R_{LA} substituent groups;
   (iii) a (4C)cycloalkylene-(1-2C)alkylene-group optionally substituted by one or more R_{LA} substituent groups;
each R_{LA} group present is methyl;
X_{A} is selected from: —N(Me)-, —S(O)₂N(H)— or —N(H)SO₂—;
Q is selected from formula Ia or Ib shown below:

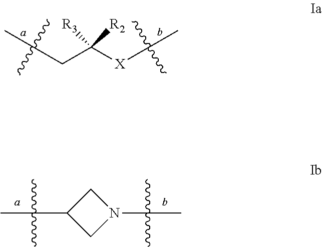

wherein:
bond a in formulae Ia and Ib corresponds with bond a in the above formula and bond b in formulae Ia and Ib corresponds with bond b in the above formula;
R₂ and R₃ are both hydrogen; and
X is NH or O;
(ii) a group of the formula:

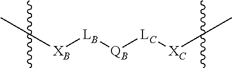

wherein:
X_{B} is selected from: —N(H)—, —N(Me)- or —S(O)₂N(H)—;
L_{B} is absent, methylene, ethylene, propylene or butylene;
Q_{B} is selected from a triazole, pyrrolidine, benzoimidazole, piperidine, phenyl, imidazole, pyrazole, oxazole or oxadiazole;
L_{C} is absent or selected from ethylene, propylene, —C(CH₃)₂CH₂CH₂ or butylene;
X_{C} is selected from: —O—, —N(H)—, —N(Me)-, —C(O)N(Me)-, —C(O)N(H)—, N(Me)C(O)— or —N(H)C(O)—;
(iii) a group of the formula:

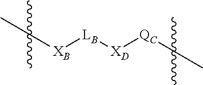

wherein:
X_{B} and L_{B} are each as defined above;
X_{D} is absent or selected from: —O—, —N(H)—, —N(Me)-, —C(O)N(H)— or —C(O)N(Me)-;
Q_{C} is a pyrrolidine, piperazine or piperidine;
(iv) a group of the formula:

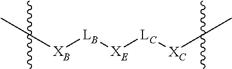

wherein:
X_{B}, L_{B}, L_{C} and X_{C} are each as defined above;
X_{E} is selected from: —O—, —N(H)—, —N(Me)-, —C(O)N(H)—, —C(O)N(Me)- —N(H)C(O)—, N(Me)C(O)— —N(H)C(O)N(H)—, N(Me)C(O)N(H)—, N(H)C(O)N(Me)-, N(Me)C(O)N(H)—, —S(O)₂N(H)—, —S(O)₂N(Me)-, —N(H)SO₂—or —N(Me)SO₂—;
wherein X_{E} is not —O— when X_{B} is —NH—, L_{B} is butylene, L_{C} is ethylene, and X_{C} is —NH— or —O—;
(v) a group of the formula:

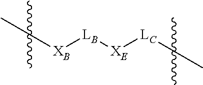

wherein:
X_{B}, L_{B}, L_{C} and X_{E} are each as defined above;
(vi) a group of the formula:

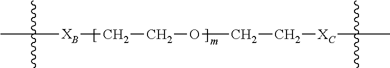

wherein:
X_{B} and X_{C} are each as defined above;
integer m is 2;
(9) L is linker [that suitably separates R_{L} from R_{R} by 7 to 13, 8 to 12, or 8, 9 or 10 bond lengths] selected from:
   (i) a group of the formula

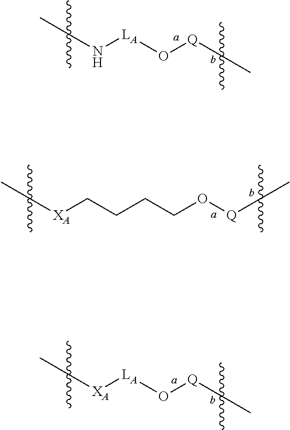

wherein L_{A} is selected from:
   (i) a (4C)alkylene substituted by one R_{LA} substituent groups;
   (ii) a propylene or a pentylene;
   (iii) a (4C)cycloalkylene-(1-2C)alkylene-group optionally substituted by one or more R_{LA} substituent groups;
each R_{LA} group present is methyl;
X_{A} is selected from: —N(Me)-, —S(O)₂N(H)— or —N(H)SO₂—;
Q is selected from formula Ia or Ib shown below:

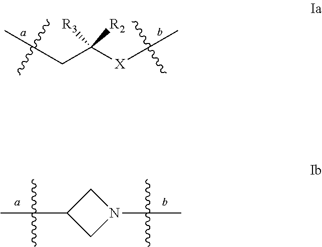

wherein:
bond a in formulae Ia and Ib corresponds with bond a in the above formula and bond b in formulae Ia and Ib corresponds with bond b in the above formula;
R₂ and R₃ are both hydrogen; and
X is NH or O;
(ii) a group of the formula:

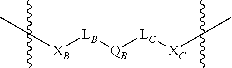

wherein:
X_{B} is selected from: —N(H)— or —S(O)₂N(H)—;
L_{B} is absent, methylene, ethylene, propylene or butylene;
Q_{B} is selected from a triazole, pyrrolidine, benzoimidazole, piperidine, phenyl, imidazole, pyrazole, oxazole or oxadiazole;
L_{C} is absent or selected from ethylene, propylene, —C(CH₃)₂CH₂CH₂— or butylene;
X_{C} is selected from: —O—, —N(H)— or —N(Me)-;
(iii) a group of the formula:

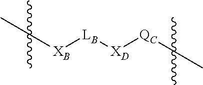

wherein:
X_{B} and L_{B} are each as defined above;
X_{D} is absent or selected from: —O—, —N(H)—;
Q_{C} is a pyrrolidine, piperazine or piperidine;
(iv) a group of the formula:

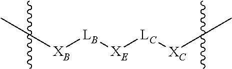

wherein:
X_{B}, L_{B}, L_{C} and X_{C} are each as defined above;
X_{E} is selected from: —O—, —N(H)—, —N(Me)-, —C(O)N(H)—, —C(O)N(Me)- —N(H)C(O)—, N(Me)C(O)— —N(H)C(O)N(H)—, N(Me)C(O)N(H)—, N(H)C(O)N(Me)-, N(Me)C(O)N(H)—, —S(O)₂N(H)—, —S(O)₂N(Me)-, —N(H)SO₂—or —N(Me)SO₂—;
wherein X_{E} is not —O— when X_{B} is —NH—, L_{B} is butylene, L_{C} is ethylene, and X_{C} is —NH— or —O—;
(v) a group of the formula:

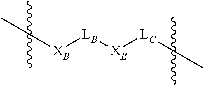

wherein:
X_{B}, L_{B}, L_{C} and X_{E} are each as defined above;
(vi) a group of the formula:

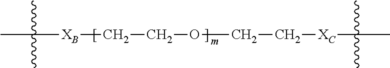

wherein:
X_{B} and X_{C} are each as defined above;
integer m is 2;
(10) L is linker selected from:

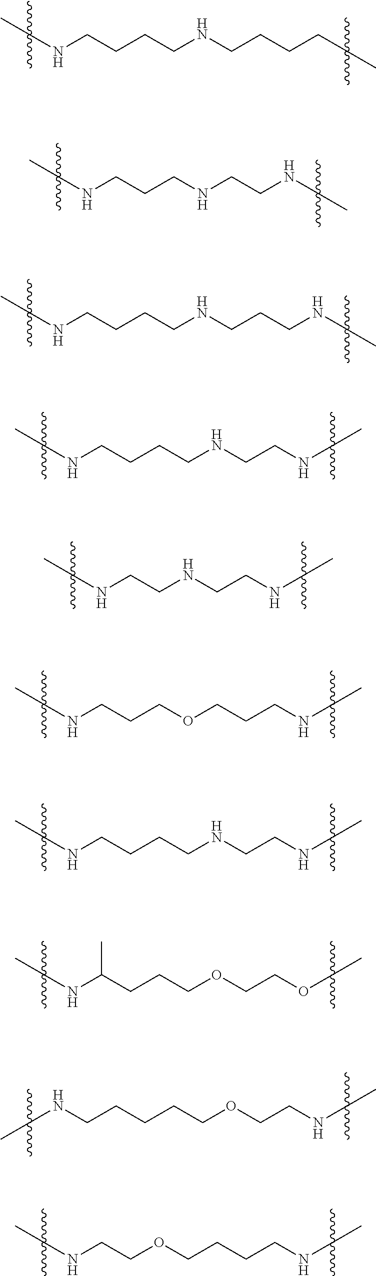

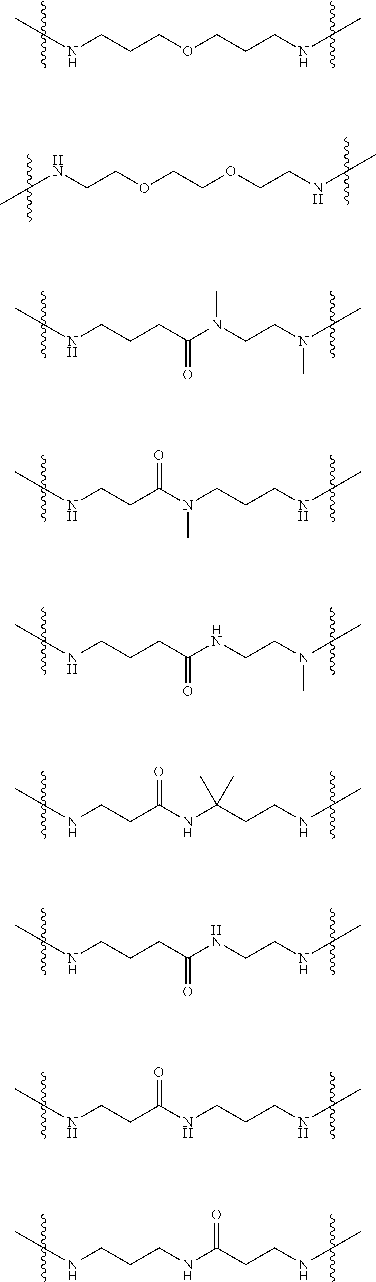

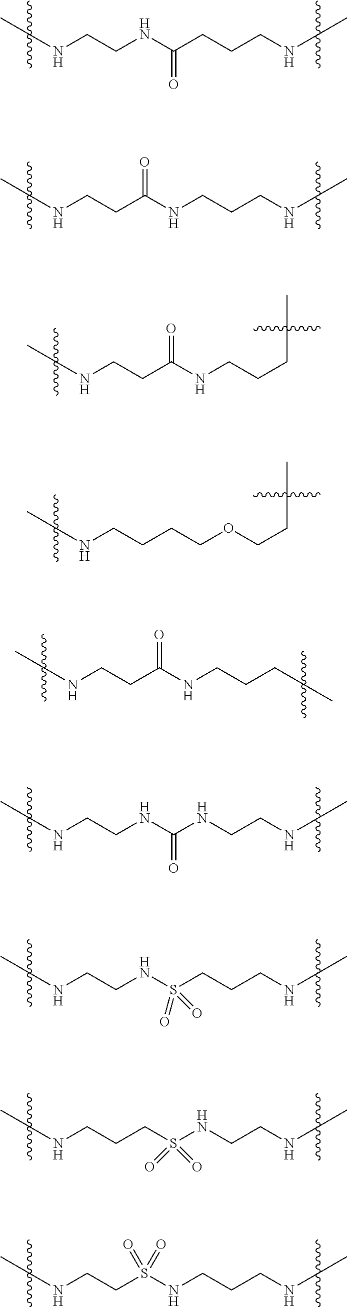

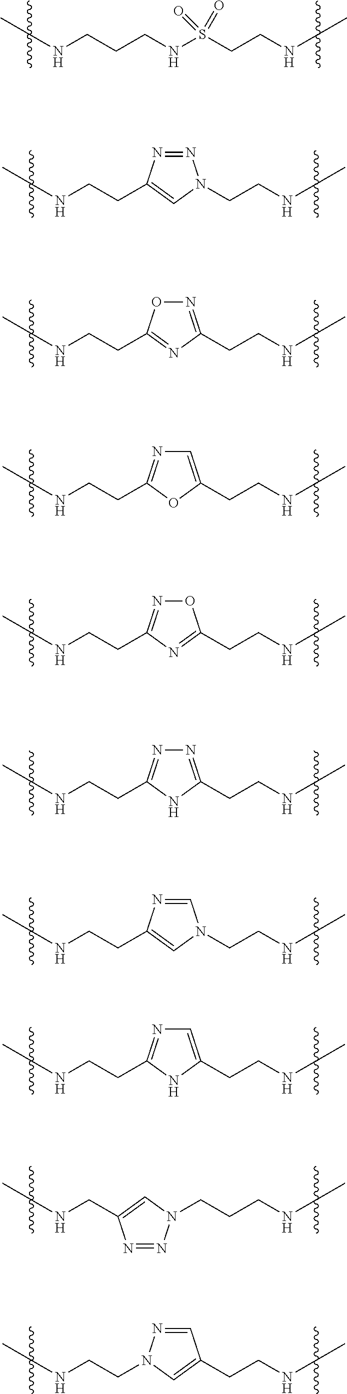

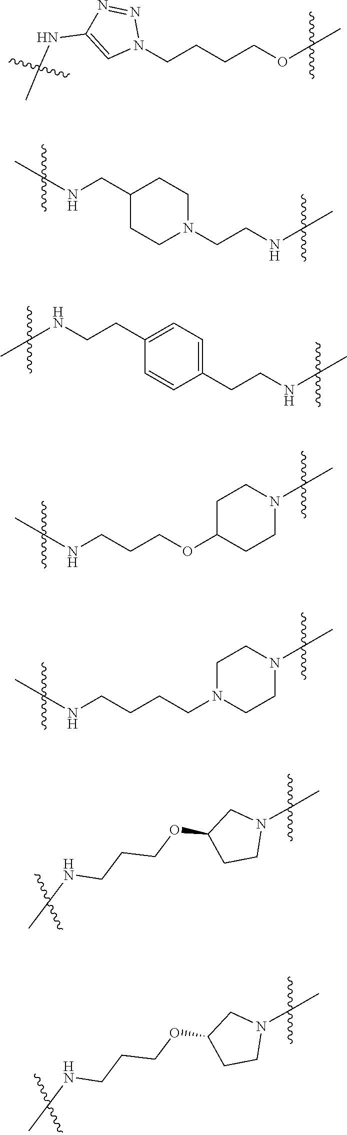

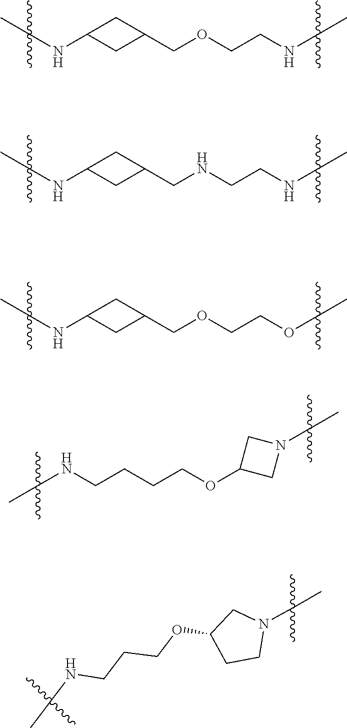 [SMILES: *NCCOCC1CC(N*)C1 ; *NCCNCC1CC(N*)C1 ; *NC1CC(COCCO*)C1 ; *NCCCCOC1CN(*)C1 ; *NCCCO[C@H]1CCN(*)C1]

(11) R_{L} is selected from one of formulae Ic, Id, Ie, If, Ig, Ih, Ii, Ij or Il shown below:

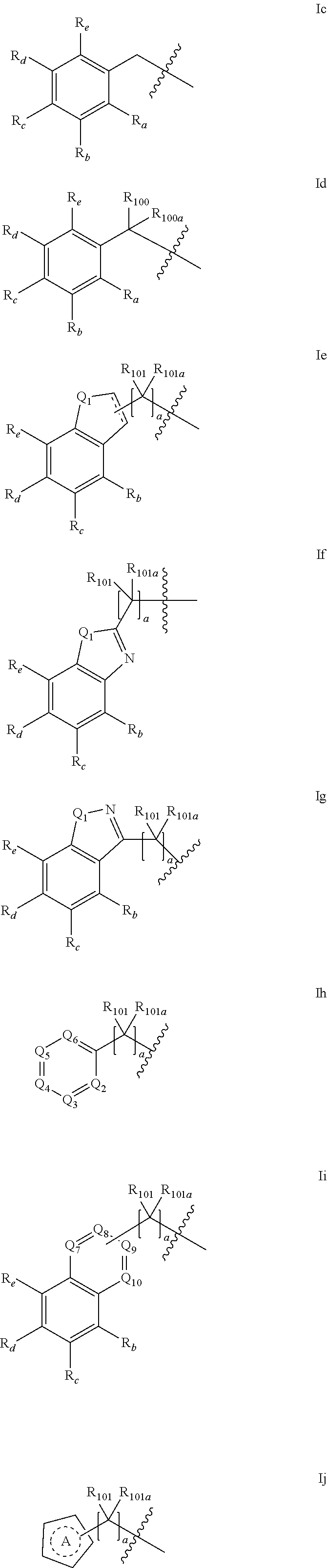

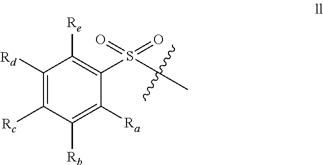

wherein:
[symbol] denotes the point of attachment; and
[symbol] denotes an optional double bond;
(12) R_{L} is selected from one of formulae Ic, Id, Ie, If, Ig, Ih, Ii or Ij shown below:

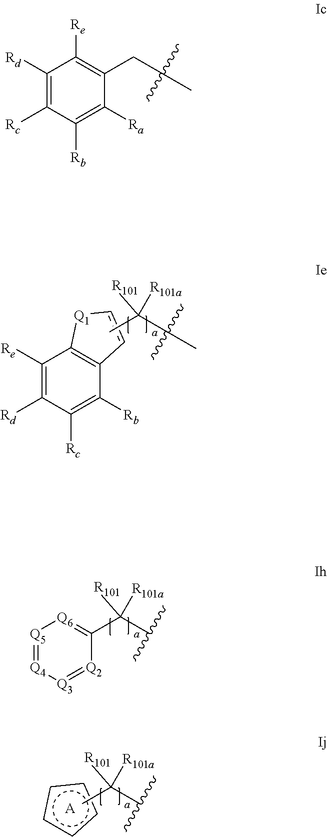

wherein:
[symbol] denotes the point of attachment; and
[symbol] denotes an optional double bond;
(13) R_{L} is selected from one of the formulae shown below:

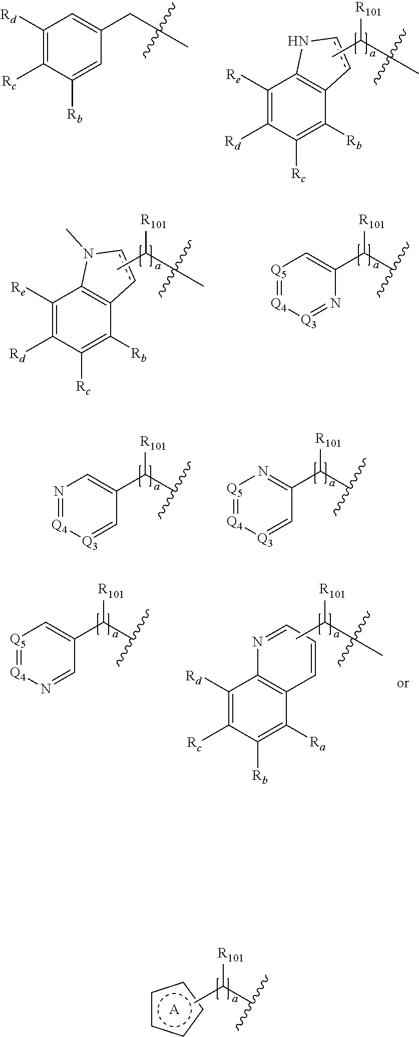

wherein:
[symbol] denotes the point of attachment; and
[symbol] denotes an optional double bond;
(14) R_{L} is selected from one of the formulae shown below:

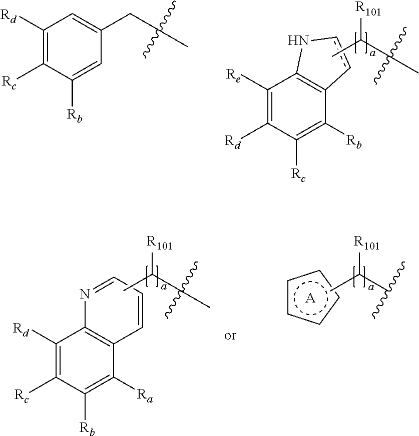

wherein:
[symbol] denotes the point of attachment; and
[symbol] denotes an optional double bond;
(15) R_{L} is selected from one of the formulae shown below:

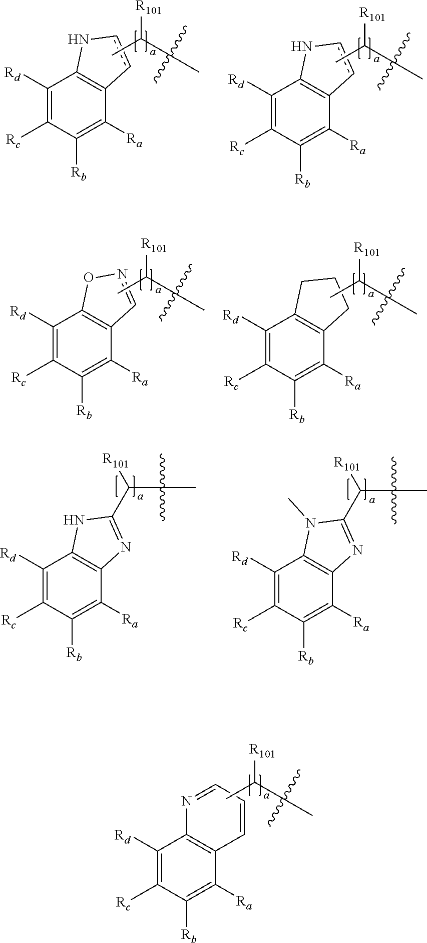

wherein:
[symbol] denotes the point of attachment; and
[symbol] denotes an optional double bond;
(16) Rₐ and Rₑ are each independently selected from hydrogen, methyl, fluoro, chloro or bromo;
(17) Rₐ and Rₑ are each independently selected from hydrogen, fluoro, chloro or bromo;
(18) Rₐ and Rₑ are each independently selected from hydrogen or chloro;
(19) Rₐ and Rₑ are both hydrogen;
(20) R_{b} and R_{d} are each independently selected from hydrogen, halo, cyano, (1-4C)alkyl, (3-6C)cycloalkyl
   —[CH₂]₀₋₂-(1-4C)alkoxy,
   —[CH₂]₀₋₂—C(O)NH₂,
   —[CH₂]₀₋₂—C(O)NH(1-4C)alkyl,
   —[CH₂]₀₋₂—C(O)N[(1-4C)alkyl]₂,
   —[CH₂]₀₋₂—NH(1-4C)alkyl,
   —[CH₂]₀₋₂—N[(1-4C)alkyl]₂,
   —[CH₂]₀₋₂—S(O)_{q}-(1-4C)alkyl (wherein q is 0, 1 or 2),
   —[CH₂]₀₋₂—C(O) (1-4C)alkyl,
   —[CH₂]₀₋₂—C(O)OH,
   —[CH₂]₀₋₂—C(O)O-(1-4C)alkyl,
   —[CH₂]₀₋₂—N(H)C(O)-(1-4C)alkyl,
   —[CH₂]₀₋₂—S(O)₂NH(1-4C)alkyl,
   —[CH₂]₀₋₂—S(O)₂N[(1-4C)alkyl]₂,
   —[CH₂]₀₋₂—N(H)SO₂-(1-4C)alkyl,
   a group of the formula:

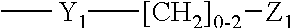

wherein Y₁ is absent, —O—, —NH—, —NMe-, —S—, —S(O)— or —S(O)₂—; and
Z₁ is (3-6C)cycloalkyl, phenyl, a 4- to 6-membered heterocyclyl or 5 or 6-membered heteroaryl;
and wherein:
any alkyl, alkoxy, cycloalkyl or —[CH₂]— moiety within a R_{b} and R_{d} substituent group is optionally substituted by one or more substituents selected from halo, hydroxy, cyano, amino, —C(O)OH, —C(O)NH₂, (1-2C)alkoxy, or (3-4C)cycloalkoxy; and
Z₁ is optionally substituted by one or more substituents selected from: halo, hydroxy, cyano, amino, —C(O)OH, —C(O)NH₂, (1-2C)alkoxy, (1-2C)alkyl, (3-4C)cycloalkyl, (3-4C)cycloalkoxy, —C(O)NH(1-2C)alkyl, —C(O)N[(1-2C)alkyl]₂, —NH(1-2C)alkyl, —N[(1-2C)alkyl]₂, —S(O)_{q}-(1-2C)alkyl (wherein q is 0, 1 or 2), —C(O)(1-2C)alkyl, —C(O)O-(1-2C)alkyl, —N(H)C(O)-(1-2C)alkyl, —S(O)₂NH(1-2C)alkyl, —S(O)₂N[(1-2C)alkyl]₂, or —NHSO₂-(1-2C)alkyl, and wherein any (1-2C)alkoxy, (1-2C)alkyl, (3-4C)cycloalkyl or (3-4C)cycloalkoxy group is optionally substituted by one or more substituents selected from halo, cyano, hydroxy, (1-2C)alkyl, (1-2C)alkoxy or (1-2C)alkoxy-(1-2C)alkyl;
(21) R_{b} and R_{d} are each independently selected from hydrogen, halo, cyano, (1-4C)alkyl, (3-6C)cycloalkyl,
   —[CH₂]₀₋₂-(1-4C)alkoxy,
   —[CH₂]₀₋₂—C(O)NH₂,
   —[CH₂]₀₋₂—C(O)NH(1-2C)alkyl,
   —[CH₂]₀₋₂—C(O)N[(1-2C)alkyl]₂,
   —[CH₂]₀₋₂—NH(1-2C)alkyl,
   —[CH₂]₀₋₂—N[(1-2C)alkyl]₂,
   —[CH₂]₀₋₂—S(O)_{q}-(1-2C)alkyl (wherein q is 0, 1 or 2),
   —[CH₂]₀₋₂—C(O)(1-2C)alkyl,
   —[CH₂]₀₋₂—C(O)OH,
   —[CH₂]₀₋₂—C(O)O-(1-2C)alkyl,
   —[CH₂]₀₋₂—N(H)C(O)-(1-2C)alkyl,
   —[CH₂]₀₋₂—S(O)₂NH(1-2C)alkyl,
   —[CH₂]₀₋₂—S(O)₂N[(1-2C)alkyl]₂,
   —[CH₂]₀₋₂—N(H)SO₂-(1-2C)alkyl,
   a group of the formula:

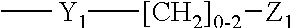

wherein Y₁ is absent, —O—, —NH—, —NMe-, —S—, —S(O)— or —S(O)₂—; and
Z₁ is (3-6C)cycloalkyl, phenyl, a 4- to 6-membered heterocyclyl or 5 or 6-membered heteroaryl;
and wherein:
any alkyl, alkoxy, cycloalkyl or —[CH₂]— moiety within a R_{b} and R_{d} substituent group is optionally substituted by one or more substituents selected from halo, hydroxy, cyano, amino, —C(O)OH, —C(O)NH₂, (1-2C)alkoxy, or (3-4C)cycloalkoxy; and
Z₁ is optionally substituted by one or more substituents selected from: halo, hydroxy, cyano, amino, —C(O)OH, —C(O)NH₂, (1-2C)alkoxy, (1-2C)alkyl, (3-4C)cycloalkyl, (3-4C)cycloalkoxy, —C(O)NH(1-2C)alkyl, —C(O)N[(1-2C)alkyl]₂, —NH(1-2C)alkyl, —N[(1-2C)alkyl]₂, —S(O)_{q}-(1-2C)alkyl (wherein q is 0, 1 or 2), —C(O)(1-2C)alkyl, —C(O)O-(1-2C)alkyl, —N(H)C(O)-(1-2C)alkyl, —S(O)₂NH(1-2C)alkyl, —S(O)₂N[(1-2C)alkyl]₂, or —NHSO₂-(1-2C)alkyl, and wherein any (1-2C)alkoxy, (1-2C)alkyl, (3-4C)cycloalkyl or (3-4C)cycloalkoxy group is optionally substituted by one or more substituents selected from halo, cyano, hydroxy, (1-2C)alkyl, (1-2C)alkoxy or (1-2C)alkoxy-(1-2C)alkyl;
(22) R_{b} and R_{d} are each independently selected from hydrogen, halo, cyano, (1-4C)alkyl, (3-6C)cycloalkyl,
   —[CH₂]₀₋₁-(1-4C)alkoxy,
   —[CH₂]₀₋₁—C(O)NH₂,
   —[CH₂]₀₋₁—C(O)NH(1-2C)alkyl,
   —[CH₂]₀₋₁—C(O)N[(1-2C)alkyl]₂,
   —[CH₂]₀₋₁—NH(1-2C)alkyl,
   —[CH₂]₀₋₁—N[(1-2C)alkyl]₂,
   —[CH₂]₀₋₁—S(O)_{q}-(1-2C)alkyl (wherein q is 0, 1 or 2),
   —[CH₂]₀₋₁—C(O) (1-2C)alkyl,
   —[CH₂]₀₋₁—C(O)OH,
   —[CH₂]₀₋₁—C(O)O-(1-2C)alkyl,
   —[CH₂]₀₋₁—N(H)C(O)-(1-2C)alkyl,
   —[CH₂]₀₋₁—S(O)₂NH(1-2C)alkyl,
   —[CH₂]₀₋₁—S(O)₂N[(1-2C)alkyl]₂,
   —[CH₂]₀₋₁—N(H)SO₂-(1-2C)alkyl,
   a group of the formula:

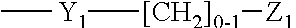

wherein Y₁ is absent, —O—, —NH—, —NMe-, —S—, —S(O)— or —S(O)₂—; and
Z₁ is (3-6C)cycloalkyl, phenyl, a 4- to 6-membered heterocyclyl or 5 or 6-membered heteroaryl;
and wherein:
any alkyl, alkoxy, cycloalkyl or —[CH₂]— moiety within a R_{b} and R_{d} substituent group is optionally substituted by one or more substituents selected from halo, hydroxy, cyano, amino, —C(O)OH, —C(O)NH₂, (1-2C)alkoxy, or (3-4C)cycloalkoxy; and
Z₁ is optionally substituted by one or more substituents selected from: halo, hydroxy, cyano, amino, —C(O)OH, —C(O)NH₂, (1-2C)alkoxy, (1-2C)alkyl, (3-4C)cycloalkyl, (3-4C)cycloalkoxy, —C(O)NH(1-2C)alkyl, —C(O)N[(1-2C)alkyl]₂, —NH(1-2C)alkyl, —N[(1-2C)alkyl]₂, —S(O)_{q}-(1-2C)alkyl (wherein q is 0, 1 or 2), —C(O)(1-2C)alkyl, —C(O)O-(1-2C)alkyl, —N(H)C(O)-(1-2C)alkyl, —S(O)₂NH(1-2C)alkyl, —S(O)₂N[(1-2C)alkyl]₂, or —NHSO₂-(1-2C)alkyl, and wherein any (1-2C)alkoxy, (1-2C)alkyl, (3-4C)cycloalkyl or (3-4C)cycloalkoxy group is optionally substituted by one or more substituents selected from halo, cyano, hydroxy, (1-2C)alkyl, (1-2C)alkoxy or (1-2C)alkoxy-(1-2C)alkyl;
(23) R_{b} and R_{d} are each independently selected from hydrogen, halo, cyano, (1-4C)alkyl, (3-6C)cycloalkyl,
   —[CH₂]₀₋₁-(1-4C)alkoxy,
   —[CH₂]₀₋₁—C(O)NH₂,
   —[CH₂]₀₋₁—C(O)NH(1-2C)alkyl,
   —[CH₂]₀₋₁—C(O)N[(1-2C)alkyl]₂,
   —[CH₂]₀₋₁—NH(1-2C)alkyl,
   —[CH₂]₀₋₁—N[(1-2C)alkyl]₂,
   —[CH₂]₀₋₁—C(O)(1-2C)alkyl,
   —[CH₂]₀₋₁—C(O)OH,
   —[CH₂]₀₋₁—C(O)O-(1-2C)alkyl,
   —[CH₂]₀₋₁—N(H)C(O)-(1-2C)alkyl,
   —[CH₂]₀₋₁—N(H)SO₂-(1-2C)alkyl,
   a group of the formula:

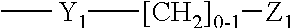

wherein Y₁ is absent, —O—, —NH—, —NMe-, —S—, —S(O)— or —S(O)₂—; and
Z₁ is (3-6C)cycloalkyl, phenyl, a 4- to 6-membered heterocyclyl or 5 or 6-membered heteroaryl;
and wherein:
any alkyl, alkoxy, cycloalkyl or —[CH₂]— moiety within a R_{b} and R_{d} substituent group is optionally substituted by one or more substituents selected from halo, hydroxy, cyano, amino, —C(O)OH, —C(O)NH₂, (1-2C)alkoxy, or (3-4C)cycloalkoxy; and
Z₁ is optionally substituted by one or more substituents selected from: halo, hydroxy, cyano, amino, —C(O)OH, —C(O)NH₂, (1-2C)alkoxy, (1-2C)alkyl, (3-4C)cycloalkyl, (3-4C)cycloalkoxy, —C(O)NH(1-2C)alkyl, —C(O)N[(1-2C)alkyl]₂, —NH(1-2C)alkyl, —N[(1-2C)alkyl]₂, —S(O)_{q}-(1-2C)alkyl (wherein q is 0, 1 or 2), —C(O)(1-2C)alkyl, —C(O)O-(1-2C)alkyl, —N(H)C(O)-(1-2C)alkyl, —S(O)₂NH(1-2C)alkyl, —S(O)₂N[(1-2C)alkyl]₂, or —NHSO₂-(1-2C)alkyl, and wherein any (1-2C)alkoxy, (1-2C)alkyl, (3-4C)cycloalkyl or (3-4C)cycloalkoxy group is optionally substituted by one or more substituents selected from halo, cyano, hydroxy, (1-2C)alkyl, (1-2C)alkoxy or (1-2C)alkoxy-(1-2C)alkyl;
(24) R_{b} and R_{d} are each independently selected from hydrogen, halo, cyano, (1-4C)alkyl, (3-6C)cycloalkyl,
   —[CH₂]₀₋₁-(1-4C)alkoxy,
   —[CH₂]₀₋₁—C(O)NH₂,
   —[CH₂]₀₋₁—C(O)NH(1-2C)alkyl,
   —[CH₂]₀₋₁—C(O)N[(1-2C)alkyl]₂,
   —[CH₂]₀₋₁—C(O) (1-2C)alkyl,
   —[CH₂]₀₋₁—C(O)OH,
   —[CH₂]₀₋₁—C(O)O-(1-2C)alkyl,
   a group of the formula:

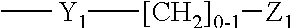

wherein Y₁ is absent, —O—, —NH—, —NMe-, —S—, —S(O)— or —S(O)₂—; and
Z₁ is (3-6C)cycloalkyl, phenyl, a 4- to 6-membered heterocyclyl or 5 or 6-membered heteroaryl;
and wherein:
any alkyl, alkoxy, cycloalkyl or —[CH₂]— moiety within a R_{b} and R_{d} substituent group is optionally substituted by one or more substituents selected from halo, hydroxy, cyano, amino, —C(O)OH, —C(O)NH₂, (1-2C)alkoxy, or (3-4C)cycloalkoxy; and
Z₁ is optionally substituted by one or more substituents selected from: halo, hydroxy, cyano, amino, —C(O)OH, —C(O)NH₂, (1-2C)alkoxy, (1-2C)alkyl, (3-4C)cycloalkyl, (3-4C)cycloalkoxy, —C(O)NH(1-2C)alkyl, —C(O)N[(1-2C)alkyl]₂, —NH(1-2C)alkyl, —N[(1-2C)alkyl]₂, —S(O)_{q}-(1-2C)alkyl (wherein q is 0, 1 or 2), —C(O)(1-2C)alkyl, —C(O)O-(1-2C)alkyl, —N(H)C(O)-(1-2C)alkyl, —S(O)₂NH(1-2C)alkyl, —S(O)₂N[(1-2C)alkyl]₂, or —NHSO₂-(1-2C)alkyl, and wherein any (1-2C)alkoxy, (1-2C)alkyl, (3-4C)cycloalkyl or (3-4C)cycloalkoxy group is optionally substituted by one or more substituents selected from halo, cyano, hydroxy, (1-2C)alkyl, (1-2C)alkoxy or (1-2C)alkoxy-(1-2C)alkyl;
(25) R_{b} and R_{d} are each independently selected from hydrogen, halo, cyano, (1-4C)alkyl, (3-6C)cycloalkyl,
   —[CH₂]₀₋₁-(1-4C)alkoxy,
   —[CH₂]₀₋₁—C(O)NH₂,
   —[CH₂]₀₋₁—C(O)NH(1-2C)alkyl,
   —[CH₂]₀₋₁—C(O)N[(1-2C)alkyl]₂,
   —[CH₂]₀₋₁—C(O)(1-2C)alkyl,
   —[CH₂]₀₋₁—C(O)OH,
   —[CH₂]₀₋₁—C(O)O-(1-2C)alkyl,
   a group of the formula:

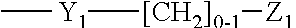

wherein Y₁ is absent, —O—, —NH—, —NMe-, —S—, —S(O)— or —S(O)₂—; and
Z₁ is (3-6C)cycloalkyl, phenyl, a 4- to 6-membered heterocyclyl or 5 or 6-membered heteroaryl;
and wherein:
any alkyl, alkoxy, cycloalkyl or —[CH₂]— moiety within a R_{b} and R_{d} substituent group is optionally substituted by one or more substituents selected from halo, hydroxy, cyano, amino, —C(O)OH, —C(O)NH₂, (1-2C)alkoxy, or (3-4C)cycloalkoxy; and
Z₁ is optionally substituted by one or more substituents selected from: halo, hydroxy, cyano, amino, —C(O)OH, —C(O)NH₂, (1-2C)alkoxy, (1-2C)alkyl, (3-4C)cycloalkyl, (3-4C)cycloalkoxy, —NH(1-2C)alkyl, —C(O)(1-2C)alkyl, —C(O)O-(1-2C)alkyl or —N(H)C(O)-(1-2C)alkyl, and wherein any (1-2C)alkoxy, (1-2C)alkyl, (3-4C)cycloalkyl or (3-4C)cycloalkoxy group is optionally substituted by one or more substituents selected from halo, cyano, hydroxy, (1-2C)alkyl, (1-2C)alkoxy or (1-2C)alkoxy-(1-2C)alkyl;
(26) R_{b} and R_{d} are each independently selected from hydrogen, halo, cyano, (1-3C)alkyl, (3-6C)cycloalkyl,
   —[CH₂]₀₋₁-(1-3C)alkoxy,
   —[CH₂]₀₋₁—C(O)NH₂,
   —[CH₂]₀₋₁—C(O)NH(1-2C)alkyl,
   a group of the formula:

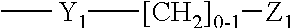

wherein Y₁ is absent, —O—, —NH—, —NMe-, —S—, —S(O)— or —S(O)₂—; and
Z₁ is (3-6C)cycloalkyl, phenyl, a 4- to 6-membered heterocyclyl or 5 or 6-membered heteroaryl;
and wherein:
any alkyl, alkoxy, cycloalkyl or —[CH₂]— moiety within a R_{b} and R_{d} substituent group is optionally substituted by one or more substituents selected from halo, hydroxy, cyano, amino, —C(O)OH, —C(O)NH₂, (1-2C)alkoxy, or (3-4C)cycloalkoxy; and
Z₁ is optionally substituted by one or more substituents selected from: halo, hydroxy, cyano, amino, —C(O)OH, —C(O)NH₂, (1-2C)alkoxy, (1-2C)alkyl, (3-4C)cycloalkyl or (3-4C)cycloalkoxy, and wherein any (1-2C)alkoxy, (1-2C)alkyl, (3-4C)cycloalkyl or (3-4C)cycloalkoxy group is optionally substituted by one or more substituents selected from halo, cyano, hydroxy, (1-2C)alkyl, (1-2C)alkoxy or (1-2C)alkoxy-(1-2C)alkyl;
(27) R_{b} and R_{d} are each independently selected from hydrogen, halo, cyano, (1-3C)alkyl,
   —[CH₂]₀₋₁-(1-3C)alkoxy,
   a group of the formula:

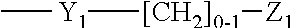

wherein Y₁ is absent, —O—, —NH— or —NMe-; and
Z₁ is (3-6C)cycloalkyl, phenyl, a 4- to 6-membered heterocyclyl or 5 or 6-membered heteroaryl;
and wherein:
any alkyl, alkoxy, cycloalkyl or —[CH₂]— moiety within a R_{b} and R_{d} substituent group is optionally substituted by one or more substituents selected from halo, hydroxy, cyano, amino, —C(O)OH, —C(O)NH₂, (1-2C)alkoxy, or (3-4C)cycloalkoxy; and
Z₁ is optionally substituted by one or more substituents selected from: halo, hydroxy, cyano, amino, —C(O)OH, —C(O)NH₂, (1-2C)alkoxy, (1-2C)alkyl, (3-4C)cycloalkyl or (3-4C)cycloalkoxy, and wherein any (1-2C)alkoxy, (1-2C)alkyl, (3-4C)cycloalkyl or (3-4C)cycloalkoxy group is optionally substituted by one or more substituents selected from halo, cyano, hydroxy, (1-2C)alkyl, (1-2C)alkoxy or (1-2C)alkoxy-(1-2C)alkyl;
(28) R_{b} and R_{d} are each independently selected from hydrogen, halo, cyano, (1-3C)alkyl,-(1-3C)alkoxy,
   a group of the formula:

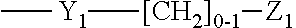

wherein Y₁ is absent, —O—, —NH— or —NMe-; and
Z₁ is (3-6C)cycloalkyl, phenyl, a 5- or 6-membered heterocyclyl or 5 or 6-membered heteroaryl;
and wherein:
any alkyl, alkoxy, cycloalkyl or —[CH₂]— moiety within a R_{b} and R_{d} substituent group is optionally substituted by one or more substituents selected from halo, hydroxy or cyano; and
Z₁ is optionally substituted by one or more substituents selected from: halo, hydroxy, cyano, amino, —C(O)OH, —C(O)NH₂, (1-2C)alkoxy, (1-2C)alkyl, (3-4C)cycloalkyl or (3-4C)cycloalkoxy, and wherein any (1-2C)alkoxy, (1-2C)alkyl, (3-4C)cycloalkyl or (3-4C)cycloalkoxy group is optionally substituted by one or more substituents selected from halo, cyano, hydroxy, (1-2C)alkyl, (1-2C)alkoxy or (1-2C)alkoxy-(1-2C)alkyl;
(29) R_{b} and R_{d} are each independently selected from hydrogen, halo, cyano, (1-2C)alkyl,-(1-2C)alkoxy,
   a group of the formula:

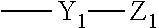

wherein Y₁ is absent, —O— —NH— or —NMe-; and
Z₁ is (3-6C)cycloalkyl or phenyl;
and wherein:
any alkyl, alkoxy, cycloalkyl or —[CH₂]— moiety within a R_{b} and R_{d} substituent group is optionally substituted by one or more substituents selected from halo, hydroxy or cyano; and
Z₁ is optionally substituted by one or more substituents selected from: halo, hydroxy, cyano, amino, —C(O)OH, —C(O)NH₂, (1-2C)alkoxy, (1-2C)alkyl, (3-4C)cycloalkyl or (3-4C)cycloalkoxy, and wherein any (1-2C)alkoxy, (1-2C)alkyl, (3-4C)cycloalkyl or (3-4C)cycloalkoxy group is optionally substituted by one or more substituents selected from halo, cyano, hydroxy, (1-2C)alkyl, (1-2C)alkoxy or (1-2C)alkoxy-(1-2C)alkyl;
(30) R_{b} and R_{d} are each independently selected from hydrogen, halo, cyano, (1-2C)alkyl,-(1-2C)alkoxy,
   a group of the formula:

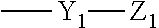

wherein Y₁ is —O—; and
Z₁ is (3-4C)cycloalkyl;
and wherein:
any alkyl, alkoxy, cycloalkyl or —[CH₂]— moiety within a R_{b} and R_{d} substituent group is optionally substituted by one or more substituents selected from halo, hydroxy or cyano; and
Z₁ is optionally substituted by one or more substituents selected from: halo, hydroxy, cyano, amino, —C(O)OH, —C(O)NH₂, (1-2C)alkoxy, (1-2C)alkyl, (3-4C)cycloalkyl or (3-4C)cycloalkoxy, and wherein any (1-2C)alkoxy, (1-2C)alkyl, (3-4C)cycloalkyl or (3-4C)cycloalkoxy group is optionally substituted by one or more substituents selected from halo, cyano, hydroxy, (1-2C)alkyl, (1-2C)alkoxy or (1-2C)alkoxy-(1-2C)alkyl;
(31) R_{b} and R_{d} are each independently selected from hydrogen, fluoro, chloro, bromo, cyano, (1-2C)alkyl,-(1-2C)alkoxy,
   a group of the formula:

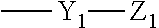

wherein Y₁ is —O—; and
Z₁ is (3-4C)cycloalkyl;
and wherein:
any alkyl, alkoxy, cycloalkyl or —[CH₂]— moiety within a R_{b} and R_{d} substituent group is optionally substituted by one or more substituents selected from halo, hydroxy or cyano;
(32) R_{b} and R_{d} are each independently selected from hydrogen, fluoro, chloro, bromo, cyano, methyl, methoxy, ethyl, ethoxy, —CH₂OH, —CH₂OCH₃, —CH₂CN, —CH₂CH₂OH, —CF₃, —OCF₃, —O—CH₂CH₂OH, —O—CH₂CF₃, —C(O)NH₂, —CH₂—C(O)NH₂, —CH(CH₃)CN, —C(CH₃)₂CN or O-cyclobutyl;
(33) R_{b} and R_{d} are each independently selected from hydrogen, fluoro, chloro, bromo, cyano, methyl, methoxy, ethyl, ethoxy, —CH₂OH, —CH₂CN, —CH₂CH₂OH, —O—CH₂CH₂OH, —CH(CH₃)CN, —C(CH₃)₂CN or O-cyclobutyl;
(34) One of R_{b} and R_{d} is hydrogen or halogen and the other is selected from any one of the options defined in paragraphs (19) to (32) above;
(35) One of R_{b} and R_{d} is hydrogen or halogen and the other is selected from hydrogen, fluoro, chloro, bromo, cyano, methyl, methoxy, ethyl, ethoxy, —CH₂OH, —CH₂CN, —CH₂CH₂OH, —O—CH₂CH₂OH, —CH(CH₃)CN, —C(CH₃)₂CN or O-cyclobutyl;
(36) R_{c} is selected from hydrogen, halo, cyano, —C(O)NH₂, (1-4C)alkyl,
   —[CH₂]₀₋₂-(1-4C)alkoxy,
   —[CH₂]₀₋₂-(3-6C)cycloalkoxy,
   —[CH₂]₀₋₂—C(O)NH₂,
   —[CH₂]₀₋₂—C(O)NH(1-4C)alkyl,
   —[CH₂]₀₋₂—C(O)N[(1-4C)alkyl]₂,
   —[CH₂]₀₋₂—NH(1-4C)alkyl,
   —[CH₂]₀₋₂—N[(1-4C)alkyl]₂,
   —[CH₂]₀₋₂—S(O)_{q}-(1-4C)alkyl (wherein q is 0, 1 or 2),
   —[CH₂]₀₋₂—C(O)(1-4C)alkyl,
   —[CH₂]₀₋₂—C(O)OH,
   —[CH₂]₀₋₂—C(O)O-(1-4C)alkyl,
   —[CH₂]₀₋₂—N(Rₕ)C(O)-(1-4C)alkyl (wherein Rₕ is hydrogen or methyl),
   —[CH₂]₀₋₂—S(O)₂NH(1-4C)alkyl,
   —[CH₂]₀₋₂—S(O)₂N[(1-4C)alkyl]₂,
   —[CH₂]₀₋₂—N(Rᵢ)SO₂-(1-4C)alkyl (wherein Rᵢ is hydrogen or methyl), a group of the formula:

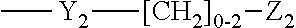

wherein Y₂ is absent, —O—, —NH—, —NMe-, —S—, —S(O)— or —S(O)₂—; and
Z₂ is (3-6C)cycloalkyl, phenyl, a 4- to 6-membered heterocyclyl or 5 or 6-membered heteroaryl;
and wherein:
   any alkyl, alkoxy, cycloalkyl or —[CH₂]— moiety within a R_{c} substituent group is optionally substituted by one or more substituents selected from halo, hydroxy, cyano, amino, —C(O)OH, —C(O)NH₂, (1-2C)alkoxy, or (3-4C)cycloalkoxy; and
   Z₂ is optionally substituted by one or more substituents selected from: halo, hydroxy, cyano, amino, —C(O)OH, —C(O)NH₂, (1-2C)alkoxy, (1-2C)alkyl, (3-4C)cycloalkyl, (3-4C)cycloalkoxy, —C(O)NH(1-2C)alkyl, —C(O)N[(1-2C)alkyl]₂, —NH(1-2C)alkyl, —N[(1-2C)alkyl]₂, —S(O)_{q}-(1-2C)alkyl (wherein q is 0, 1 or 2), —C(O)(1-2C)alkyl, —C(O)O-(1-2C)alkyl, —N(R_{f})C(O)-(1-2C)alkyl, —S(O)₂NH(1-2C)alkyl, —S(O)₂N[(1-2C)alkyl]₂, or —NHSO₂-(1-2C)alkyl, and wherein any (1-2C)alkoxy, (1-2C)alkyl, (3-4C)cycloalkyl or (3-4C)cycloalkoxy group is optionally substituted by one or more substituents selected from halo, cyano, hydroxy, (1-2C)alkyl, (1-2C)alkoxy or (1-2C)alkoxy-(1-2C)alkyl;
(37) R_{c} is selected from hydrogen, halo, cyano, —C(O)NH₂, (1-3C)alkyl,
   —[CH₂]₀₋₂-(1-3C)alkoxy,
   —[CH₂]₀₋₂-(3-6C)cycloalkoxy,
   —[CH₂]₀₋₂—C(O)NH₂,
   —[CH₂]₀₋₂—C(O)NH(1-3C)alkyl,
   —[CH₂]₀₋₂—C(O)N[(1-3C)alkyl]₂,
   —[CH₂]₀₋₂—NH(1-3C)alkyl,
   —[CH₂]₀₋₂—N[(1-3C)alkyl]₂,
   —[CH₂]₀₋₂—S(O)_{q}-(1-3C)alkyl (wherein q is 0, 1 or 2),
   —[CH₂]₀₋₂—C(O)(1-3C)alkyl,
   —[CH₂]₀₋₂—C(O)OH,
   —[CH₂]₀₋₂—C(O)O-(1-3C)alkyl,
   —[CH₂]₀₋₂—N(H)C(O)-(1-3C)alkyl,
   —[CH₂]₀₋₂—S(O)₂NH(1-3C)alkyl,
   —[CH₂]₀₋₂—S(O)₂N[(1-3C)alkyl]₂,
   —[CH₂]₀₋₂—N(H)SO₂-(1-3C)alkyl,
   a group of the formula:

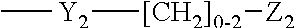

wherein Y₂ is absent, —O—, —NH—, —NMe-, —S—, —S(O)— or —S(O)₂—; and
Z₂ is (3-6C)cycloalkyl, phenyl, a 4- to 6-membered heterocyclyl or 5 or 6-membered heteroaryl;
and wherein:
   any alkyl, alkoxy, cycloalkyl or —[CH₂]— moiety within a R_{c} substituent group is optionally substituted by one or more substituents selected from halo, hydroxy, cyano, amino, —C(O)OH, —C(O)NH₂, (1-2C)alkoxy, or (3-4C)cycloalkoxy; and
   Z₂ is optionally substituted by one or more substituents selected from: halo, hydroxy, cyano, amino, —C(O)OH, —C(O)NH₂, (1-2C)alkoxy, (1-2C)alkyl, (3-4C)cycloalkyl, (3-4C)cycloalkoxy, —C(O)NH(1-2C)alkyl, —C(O)N[(1-2C)alkyl]₂, —NH(1-2C)alkyl, —N[(1-2C)alkyl]₂, —S(O)_{q}-(1-2C)alkyl (wherein q is 0, 1 or 2), —C(O)(1-2C)alkyl, —C(O)O-(1-2C)alkyl, —N(R_{f})C(O)-(1-2C)alkyl, —S(O)₂NH(1-2C)alkyl, —S(O)₂N[(1-2C)alkyl]₂, or —NHSO₂-(1-2C)alkyl, and wherein any (1-2C)alkoxy, (1-2C)alkyl, (3-4C)cycloalkyl or (3-4C)cycloalkoxy group is optionally substituted by one or more substituents selected from halo, cyano, hydroxy, (1-2C)alkyl, (1-2C)alkoxy or (1-2C)alkoxy-(1-2C)alkyl;
(38) R_{c} is selected from hydrogen, halo, cyano, —C(O)NH₂, (1-3C)alkyl,
   —[CH₂]₀₋₁-(1-3C)alkoxy,
   —[CH₂]₀₋₁-(3-6C)cycloalkoxy,
   —[CH₂]₀₋₁—C(O)NH₂,
   —[CH₂]₀₋₁—C(O)NH(1-3C)alkyl,
   —[CH₂]₀₋₁—C(O)N[(1-3C)alkyl]₂,
   —[CH₂]₀₋₁—NH(1-3C)alkyl,
   —[CH₂]₀₋₁—N[(1-3C)alkyl]₂,
   —[CH₂]₀₋₁—S(O)_{q}-(1-3C)alkyl (wherein q is 0, 1 or 2),
   —[CH₂]₀₋₁—C(O) (1-3C)alkyl,
   —[CH₂]₀₋₁—C(O)OH,
   —[CH₂]₀₋₁—C(O)O-(1-3C)alkyl,
   —[CH₂]₀₋₁—N(H)C(O)-(1-3C)alkyl,
   —[CH₂]₀₋₁—S(O)₂NH(1-3C)alkyl,
   —[CH₂]₀₋₁—S(O)₂N[(1-3C)alkyl]₂,
   —[CH₂]₀₋₁—N(H)SO₂-(1-3C)alkyl,
   a group of the formula:

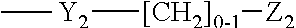

wherein Y₂ is absent, —O—, —NH—, —NMe-, —S—, —S(O)— or —S(O)₂—; and
Z₂ is (3-6C)cycloalkyl, phenyl, a 4- to 6-membered heterocyclyl or 5 or 6-membered heteroaryl;
and wherein:
   any alkyl, alkoxy, cycloalkyl or —[CH₂]— moiety within a R_{c} substituent group is optionally substituted by one or more substituents selected from halo, hydroxy, cyano, amino, —C(O)OH, —C(O)NH₂, (1-2C)alkoxy, or (3-4C)cycloalkoxy; and
   Z₂ is optionally substituted by one or more substituents selected from: halo, hydroxy, cyano, amino, —C(O)OH, —C(O)NH₂, (1-2C)alkoxy, (1-2C)alkyl, (3-4C)cycloalkyl, (3-4C)cycloalkoxy, —C(O)NH(1-2C)alkyl, —C(O)N[(1-2C)alkyl]₂, —NH(1-2C)alkyl, —N[(1-2C)alkyl]₂, —S(O)_{q}-(1-2C)alkyl (wherein q is 0, 1 or 2), —C(O)(1-2C)alkyl, —C(O)O-(1-2C)alkyl, —N(R_{f})C(O)-(1-2C)alkyl, —S(O)₂NH(1-2C)alkyl, —S(O)₂N[(1-2C)alkyl]₂, or —NHSO₂-(1-2C)alkyl, and wherein any (1-2C)alkoxy, (1-2C)alkyl, (3-4C)cycloalkyl or (3-4C)cycloalkoxy group is optionally substituted by one or more substituents selected from halo, cyano, hydroxy, (1-2C)alkyl, (1-2C)alkoxy or (1-2C)alkoxy-(1-2C)alkyl;
(39) R_{c} is selected from hydrogen, halo, cyano, —C(O)NH₂, (1-3C)alkyl,
   —[CH₂]₀₋₁-(1-3C)alkoxy,
   —[CH₂]₀₋₁-(3-6C)cycloalkoxy,
   —[CH₂]₀₋₁—C(O)NH₂,
   —[CH₂]₀₋₁—C(O)NH(1-3C)alkyl,
   —[CH₂]₀₋₁—C(O)N[(1-3C)alkyl]₂,
   —[CH₂]₀₋₁—NH(1-3C)alkyl,
   —[CH₂]₀₋₁—N[(1-3C)alkyl]₂,
   —[CH₂]₀₋₁—C(O)(1-3C)alkyl,
   —[CH₂]₀₋₁—C(O)OH,
   —[CH₂]₀₋₁—C(O)O-(1-3C)alkyl,
   —[CH₂]₀₋₁—N(H)C(O)-(1-3C)alkyl,
   a group of the formula:

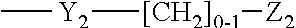

wherein Y₂ is absent, —O—, —NH—, —NMe-, —S—, —S(O)— or —S(O)₂—; and
Z₂ is (3-6C)cycloalkyl, phenyl, a 4- to 6-membered heterocyclyl or 5 or 6-membered heteroaryl;
and wherein:
   any alkyl, alkoxy, cycloalkyl or —[CH₂]— moiety within a R_{c} substituent group is optionally substituted by one or more substituents selected from halo, hydroxy, cyano, amino, —C(O)OH, —C(O)NH₂, (1-2C)alkoxy, or (3-4C)cycloalkoxy; and
   Z₂ is optionally substituted by one or more substituents selected from: halo, hydroxy, cyano, amino, —C(O)OH, —C(O)NH₂, (1-2C)alkoxy, (1-2C)alkyl, (3-4C)cycloalkyl, (3-4C)cycloalkoxy, —C(O)NH(1-2C)alkyl, —C(O)N[(1-2C)alkyl]₂, —NH(1-2C)alkyl, —N[(1-2C)alkyl]₂, —S(O)_{q}-(1-2C)alkyl (wherein q is 0, 1 or 2), —C(O)(1-2C)alkyl, —C(O)O-(1-2C)alkyl, —N(R_{f})C(O)-(1-2C)alkyl, —S(O)₂NH(1-2C)alkyl, —S(O)₂N[(1-2C)alkyl]₂, or —NHSO₂-(1-2C)alkyl, and wherein any (1-2C)alkoxy, (1-2C)alkyl, (3-4C)cycloalkyl or (3-4C)cycloalkoxy group is optionally substituted by one or more substituents selected from halo, cyano, hydroxy, (1-2C)alkyl, (1-2C)alkoxy or (1-2C)alkoxy-(1-2C)alkyl;
(40) R_{c} is selected from hydrogen, halo, cyano, —C(O)NH₂, (1-3C)alkyl,
   —[CH₂]₀₋₁-(1-3C)alkoxy,
   —[CH₂]₀₋₁-(4-6C)cycloalkoxy,
   —[CH₂]₀₋₁—C(O)NH₂,
   —[CH₂]₀₋₁—NH(1-3C)alkyl,
   —[CH₂]₀₋₁—C(O)(1-3C)alkyl,
   —[CH₂]₀₋₁—C(O)OH,
   —[CH₂]₀₋₁—C(O)O-(1-3C)alkyl,
   —[CH₂]₀₋₁—N(H)C(O)-(1-3C)alkyl,
   a group of the formula:

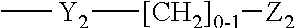

wherein Y₂ is absent, —O—, —NH—, —NMe-, —S—, —S(O)— or —S(O)₂—; and
Z₂ is (3-6C)cycloalkyl, phenyl, a 4- to 6-membered heterocyclyl or 5 or 6-membered heteroaryl;
and wherein:
   any alkyl, alkoxy, cycloalkyl or —[CH₂]— moiety within a R_{c} substituent group is optionally substituted by one or more substituents selected from halo, hydroxy, cyano, amino, —C(O)OH, —C(O)NH₂, (1-2C)alkoxy, or (3-4C)cycloalkoxy; and
   Z₂ is optionally substituted by one or more substituents selected from: halo, hydroxy, cyano, amino, —C(O)OH, —C(O)NH₂, (1-2C)alkoxy, (1-2C)alkyl, (3-4C)cycloalkyl, (3-4C)cycloalkoxy, —C(O)NH(1-2C)alkyl, —C(O)N[(1-2C)alkyl]₂, —NH(1-2C)alkyl, —N[(1-2C)alkyl]₂, —S(O)_{q}-(1-2C)alkyl (wherein q is 0, 1 or 2), —C(O)(1-2C)alkyl, —C(O)O-(1-2C)alkyl, —N(R_{f})C(O)-(1-2C)alkyl, —S(O)₂NH(1-2C)alkyl, —S(O)₂N[(1-2C)alkyl]₂, or —NHSO₂-(1-2C)alkyl, and wherein any (1-2C)alkoxy, (1-2C)alkyl, (3-4C)cycloalkyl or (3-4C)cycloalkoxy group is optionally substituted by one or more substituents selected from halo, cyano, hydroxy, (1-2C)alkyl, (1-2C)alkoxy or (1-2C)alkoxy-(1-2C)alkyl;
(41) R_{c} is selected from hydrogen, halo, cyano, —C(O)NH₂, (1-3C)alkyl,
   —[CH₂]₀₋₁-(1-3C)alkoxy,
   a group of the formula:

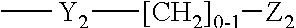

wherein Y₂ is absent, —O—, —NH—, —NMe-, —S—, —S(O)— or —S(O)₂—; and
Z₂ is (3-6C)cycloalkyl, phenyl, a 4- to 6-membered heterocyclyl or 5 or 6-membered heteroaryl;
and wherein:
   any alkyl, alkoxy, cycloalkyl or —[CH₂]— moiety within a R_{c} substituent group is optionally substituted by one or more substituents selected from halo, hydroxy, cyano, amino, —C(O)OH, —C(O)NH₂, (1-2C)alkoxy, or (3-4C)cycloalkoxy; and
   Z₂ is optionally substituted by one or more substituents selected from: halo, hydroxy, cyano, amino, —C(O)OH, —C(O)NH₂, (1-2C)alkoxy, (1-2C)alkyl, (3-4C)cycloalkyl, (3-4C)cycloalkoxy, —C(O)NH(1-2C)alkyl, —C(O)N[(1-2C)alkyl]₂, —NH(1-2C)alkyl, —N[(1-2C)alkyl]₂, —S(O)_{q}-(1-2C)alkyl (wherein q is 0, 1 or 2), —C(O)(1-2C)alkyl, —C(O)O-(1-2C)alkyl, —N(R)C(O)-(1-2C)alkyl, —S(O)₂NH(1-2C)alkyl, —S(O)₂N[(1-2C)alkyl]₂, or —NHSO₂-(1-2C)alkyl, and wherein any (1-2C)alkoxy, (1-2C)alkyl, (3-4C)cycloalkyl or (3-4C)cycloalkoxy group is optionally substituted by one or more substituents selected from halo, cyano, hydroxy, (1-2C)alkyl, (1-2C)alkoxy or (1-2C)alkoxy-(1-2C)alkyl;
(42) R_{c} is selected from hydrogen, halo, cyano, —C(O)NH₂, (1-3C)alkyl, -(1-3C)alkoxy,
   a group of the formula:

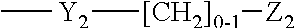

wherein Y₂ is absent, —O—, —NH— or —NMe-; and
Z₂ is (3-6C)cycloalkyl, phenyl, a 5 or 6-membered heterocyclyl or 5 or 6-membered heteroaryl;
and wherein:
   any alkyl, alkoxy, cycloalkyl or —[CH₂]— moiety within a R_{c} substituent group is optionally substituted by one or more substituents selected from halo, hydroxy, cyano, amino, —C(O)OH, —C(O)NH₂, (1-2C)alkoxy, or (3-4C)cycloalkoxy; and
   Z₂ is optionally substituted by one or more substituents selected from: halo, hydroxy, cyano, amino, —C(O)OH, —C(O)NH₂, (1-2C)alkoxy, (1-2C)alkyl, (3-4C)cycloalkyl, (3-4C)cycloalkoxy, —C(O)NH (Me), —C(O)N(Me) 2, —NH (Me), —N(Me)₂, —S(O)_{q}-(Me) (wherein q is 0, 1 or 2), —C(O) (Me), —C(O)O-(Me), —N(R_{f})C(O)-(Me), —S(O)₂NH(Me), —S(O)₂N(Me)₂, or —NHSO₂-(Me), and wherein any (1-2C)alkoxy, (1-2C)alkyl, (3-4C)cycloalkyl or (3-4C)cycloalkoxy group is optionally substituted by one or more substituents selected from halo, cyano, hydroxy, (1-2C)alkyl, (1-2C)alkoxy or (1-2C)alkoxy-(1-2C)alkyl;
(43) R_{c} is selected from hydrogen, halo, (1-3C)alkyl, -(1-3C)alkoxy,
   a group of the formula:

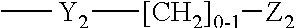

wherein Y₂ is absent or —O—; and
Z₂ is (3-6C)cycloalkyl, phenyl or 6-membered heteroaryl;
and wherein:
   any alkyl, alkoxy, cycloalkyl or —[CH₂]— moiety within a R_{c} substituent group is optionally substituted by one or more substituents selected from halo, hydroxy, cyano, amino, —C(O)OH, —C(O)NH₂, (1-2C)alkoxy, or (3-4C)cycloalkoxy; and
   Z₂ is optionally substituted by one or more substituents selected from: halo, hydroxy, cyano, amino, —C(O)OH, —C(O)NH₂, (1-2C)alkoxy, (1-2C)alkyl, (3-4C)cycloalkyl or (3-4C)cycloalkoxy and wherein any (1-2C)alkoxy, (1-2C)alkyl, (3-4C)cycloalkyl or (3-4C)cycloalkoxy group is optionally substituted by one or more substituents selected from halo, cyano, hydroxy, (1-2C)alkyl, (1-2C)alkoxy or (1-2C)alkoxy-(1-2C)alkyl;
(44) R_{c} is selected from hydrogen, fluoro, chloro, bromo, methyl, ethyl, methoxy, ethoxy,
   a group of the formula:

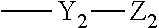

wherein Y₂ is absent or —O—; and
Z₂ is (3-6C)cycloalkyl, phenyl or 6-membered heteroaryl;
and wherein:
   any alkyl, alkoxy, cycloalkyl or —[CH₂]— moiety within a R_{c} substituent group is optionally substituted by one or more substituents selected from halo, hydroxy, cyano or amino; and
   Z₂ is optionally substituted by one or more substituents selected from: halo, hydroxy, cyano, amino, —C(O)OH, —C(O)NH₂, (1-2C)alkoxy, (1-2C)alkyl, (3-4C)cycloalkyl or (3-4C)cycloalkoxy and wherein any (1-2C)alkoxy, (1-2C)alkyl, (3-4C)cycloalkyl or (3-4C)cycloalkoxy group is optionally substituted by one or more substituents selected from halo, cyano, hydroxy, (1-2C)alkyl, (1-2C)alkoxy or (1-2C)alkoxy-(1-2C)alkyl;
(45) R_{c} is selected from hydrogen, fluoro, chloro, bromo, methyl, ethyl, methoxy, ethoxy,
   a group of the formula:

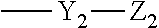

wherein Y₂ is absent or —O—; and
Z₂ is cyclopropyl, cyclobutyl, cyclopentyl, phenyl, pyridine, pyrazine or pyrimidine;
and wherein:
   any alkyl, alkoxy, cycloalkyl or —[CH₂]— moiety within a R_{c} substituent group is optionally substituted by one or more substituents selected from halo, hydroxy, cyano or amino; and
   Z₂ is optionally substituted by one or more substituents selected from: halo, hydroxy, cyano, amino, —C(O)OH, —C(O)NH₂, (1-2C)alkoxy, (1-2C)alkyl, (3-4C)cycloalkyl or (3-4C)cycloalkoxy and wherein any (1-2C)alkoxy, (1-2C)alkyl, (3-4C)cycloalkyl or (3-4C)cycloalkoxy group is optionally substituted by one or more substituents selected from halo, cyano, hydroxy, (1-2C)alkyl, (1-2C)alkoxy or (1-2C)alkoxy-(1-2C)alkyl;
(46) R_{c} is selected from hydrogen, fluoro, chloro, bromo, methyl, ethyl, methoxy, ethoxy,
   a group of the formula:

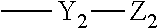

wherein Y₂ is absent or —O—; and
Z₂ is cyclopropyl, cyclobutyl, cyclopentyl, phenyl, pyridine, pyrazine or pyrimidine;
and wherein:
   any alkyl, alkoxy, cycloalkyl or —[CH₂]— moiety within a R_{c} substituent group is optionally substituted by one or more substituents selected from fluoro, chloro, bromo or hydroxy; and
   Z₂ is optionally substituted by one or more substituents selected from: halo, hydroxy, (1-2C)alkoxy, (1-2C)alkyl, (3-4C)cycloalkyl or (3-4C)cycloalkoxy and wherein any (1-2C)alkoxy, (1-2C)alkyl, (3-4C)cycloalkyl or (3-4C)cycloalkoxy group is optionally substituted by one or more substituents selected from fluoro, chloro, cyano or hydroxy;
(47) R_{c} is selected from hydrogen, fluoro, chloro, methyl, methoxy,
   a group of the formula:

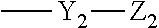

wherein Y₂ is absent or —O—; and
Z₂ is cyclopropyl, cyclobutyl, cyclopentyl, phenyl, pyridine, pyrazine or pyrimidine;
and wherein:
   any alkyl, alkoxy, cycloalkyl or —[CH₂]— moiety within a R_{c} substituent group is optionally substituted by one or more substituents selected from fluoro, chloro or hydroxy; and
   Z₂ is optionally substituted by one or more substituents selected from: methyl, ethyl, methoxy or ethoxy and wherein any methyl, ethyl, methoxy or ethoxy group is optionally substituted by one or more substituents selected from cyano or hydroxy;
(48) R_{c} is selected from hydrogen, fluoro, chloro, methyl, —CF₃, methoxy, —OCF₃, cyclopropyl, cyclobutyl, cyclopentyl, phenyl, pyridine, pyrazine, pyrimidine, —O-cyclopropyl, —O-cyclobutyl, —O-cyclopentyl, -phenyl-O-(1-2Calkyl)-OH, -phenyl-(1-2Calkyl)-OH or -phenyl-(1-2Calkyl)-CN;
(49) R_{c} is selected from hydrogen, fluoro, chloro, methyl, —CF₃, methoxy, —OCF₃, cyclopropyl, cyclobutyl, cyclopentyl, phenyl, pyridine, pyrazine, pyrimidine, —O-cyclopropyl, —O-cyclobutyl, —O-cyclopentyl, -phenyl-O—CH₂CH₂—OH, -phenyl-CH₂—OH, -phenyl-CH₂CH₂—OH or -phenyl-CH₂—CN;
(50) R₁₀₀ is (1-2C)alkyl optionally substituted by hydroxy;
(51) R₁₀₀ is methyl or methoxy;
(52) R₁₀₀ₐ is hydrogen;
(53) R₁₀₀ₐ is methyl;
(54) R₁₀₀ and R₁₀₀ₐ are linked to form a cyclopropyl ring;
(55) R₁₀₀ and R₁₀₀ₐ aare linked to form a cyclobutyl ring;
(56) R₁₀₁ is hydrogen or methyl optionally substituted by hydroxy or halo;
(57) R₁₀₁ is hydrogen;
(58) R₁₀₁ₐ is hydrogen;
(59) R₁₀₁ and R₁₀₁ₐ are linked to form a cyclopropyl ring;
(60) R₁₀₁ and R₁₀₁ₐ are linked to form a cyclobutyl ring;
(61) integer a is 0 or 1;
(62) Q₁ is selected from —NR₁₀₂—, —O— or —CH—, wherein R₁₀₂ is hydrogen or methyl;
(63) Q₁ is selected from —NH— or —NMe-;
(64) [symbol] represents a double bond;
(65) Q₂ is N or CRₐ;
   Q₃ is N or CR_{b};
   Q₄ is N or CR_{c};
   Q₅ is N or CR_{d};
   Q₆ is N or CRₑ;
   Rₐ, R_{b}, R_{c}, R_{d} and Rₑ are each as defined in any one of paragraphs (15) to (18), (19) to (34) and (35) to (48) above;
   with the proviso that one to three of Q₂, Q₃, Q₄, Q₅ or Q₆ is/are N;
(66) Q₂ is N or CH;
   Q₃ is N or CR_{b};
   Q₄ is N or CR_{c};
   Q₅ is N or CR_{d};
   Q₆ is N or CH;
   R_{b}, R_{c} and R_{d} are each as defined in any one of paragraphs (26) to (34) and (41) to (48) above;
   with the proviso that one or two of Q₂, Q₃, Q₄, Q₅ or Q₆ is/are N;
(67) Q₂ is N;
   Q₃ is CR_{b};
   Q₄ is CR_{c};
   Q₅ is CR_{d};
   Q₆ is CH;
   R_{b}, R_{c} and R_{d} are each as defined in any one of paragraphs (26) to (34) and (41) to (48) above;
(68) Q₂ is CH;
   Q₃ is N;
   Q₄ is CR_{c};
   Q₅ is CR_{d};
   Q₆ is CH;
   R_{c} and R_{d} are each as defined in any one of paragraphs (26) to (34) and (41) to (48) above;
(69) Q₂ is CH;
   Q₃ is CR_{b};
   Q₄ is CR_{c};
   Q₅ is N;
   Q₆ is CH;
   R_{b} and R_{c} are each as defined in any one of paragraphs (26) to (34) and (41) to (48) above;
(70) Q₂ is CH;
   Q₃ is CR_{b};
   Q₄ is CR_{c};
   Q₅ is CR_{d};
   Q₆ is N;
   R_{b}, R_{c} and R_{d} are each as defined in any one of paragraphs (26) to (34) and (41) to (48) above;
(71) Q₇ is N or CR_{f};
   Q₈ is N or CR_{f};
   Q₉ is N or CR_{f};
   Q₁₀ is N or CR_{f};
   with the proviso that one or two of Q₇, Q₈, Q₉ or Q₁₀ is/are N;
each R_{f} present is independently selected from hydrogen, methyl, fluoro, chloro or bromo;
(72) Q₇ is N or CR_{f};
   Q₈ is N or CR_{f};
   Q₉ is N or CR_{f};
   Q₁₀ is N or CR_{f};
   with the proviso that one or two of Q₇, Q₈, Q₉ or Q₁₀ is/are N;
each R_{f} present is independently selected from hydrogen
(73) Q₇ is N;
   Q₈ is CH;
   Q₉ is CH;
   Q₁₀ is CH;
(74) Ring A is a five-membered heteroaryl ring comprising one, two or three heteroatoms selected from N, O or S, optionally substituted by one R_{b} (as defined herein) and/or one or two R_{c} substituents (as defined herein);
(75) Ring A is a five-membered heteroaryl ring comprising one or two heteroatoms selected from N, O or S, optionally substituted by one R_{b} (as defined in any one of paragraphs (26) to (34) above) and/or one or two R_{c} substituents (as defined in any one of paragraphs (41) to (48) above);
(76) Ring A is a five-membered heteroaryl ring comprising one or two heteroatoms selected from N or S, optionally substituted by one R_{b} (as defined in any one of paragraphs (30) to (34) above) and/or one or two R_{c} substituents (as defined in any one of paragraphs (46) to (48) above);
(77) Ring A is a pyrazole or a thiazole optionally substituted by one R_{b} (as defined in any one of paragraphs (30) to (34) above) and/or one or two R_{c} substituents (as defined in any one of paragraphs (46) to (48) above);
(78) Ring A is a pyrazole or a thiazole substituted one, two or three groups independently selected from phenyl, methyl and chloro;
(79) R_{R} is selected from one of formulae Im or In shown below:

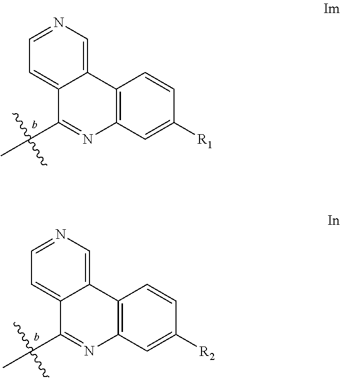

wherein:
[symbol] denotes the point of attachment;
[symbol] denotes an optional double bond;
(80) R_{R} is selected from one of formulae Io or Ip shown below:

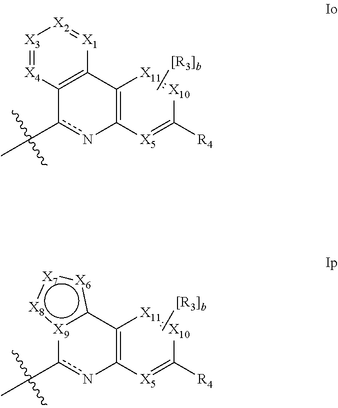

wherein:
[symbol] denotes the point of attachment;
[symbol] denotes an optional double bond;
(81) R_{R} is selected from one of formulae Iq or Ir shown below:

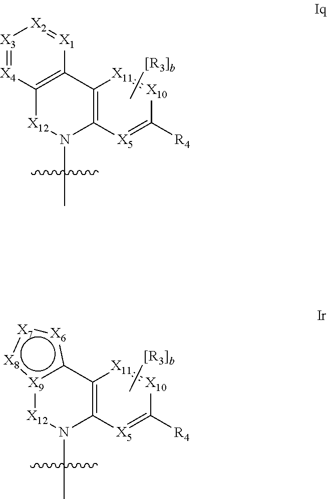

wherein:
[symbol] denotes the point of attachment;
(82) R_{R} is selected from any one of the formulae shown below:

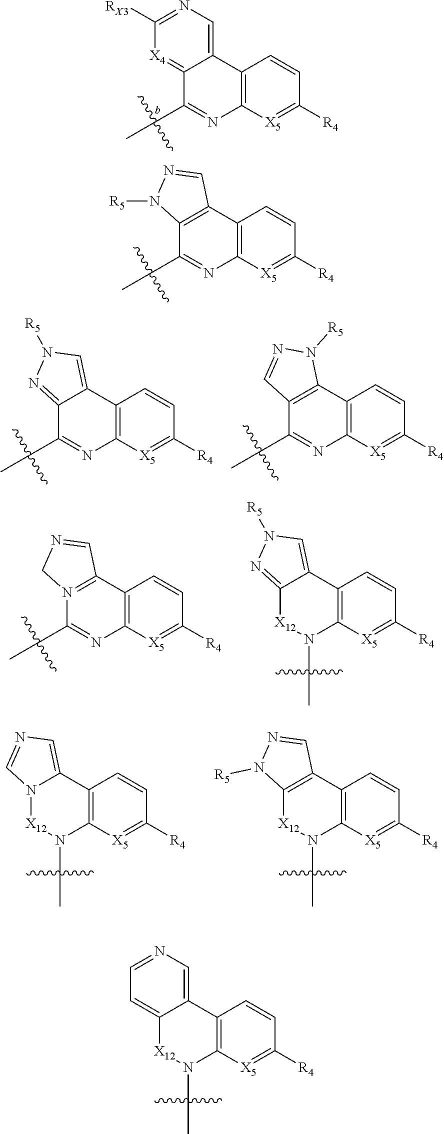

wherein:
[symbol] denotes the point of attachment; and
R₅ is hydrogen, methyl or ethyl.
(83) R₁ is —C(O)OH;
(84) R₁ is —C(O)NH₂;
(85) R₂ is selected from:
   (i) —C(O)NHR₂ₐ or —C(O)OR₂ₐ, wherein R₂ₐ is (1-3C)alkyl optionally substituted by halo, hydroxy, amino or carboxyl;
   (ii) —N(H)C(O) R_{2c}, wherein R_{2c} is (1-3C)alkyl optionally substituted by halo, hydroxy, amino or carboxyl;
   (iii) —CN;
   (iv) a 5- or 6-membered heteroaryl comprising one, two, three or four nitrogen atoms;
   (v) oxetanyl, tetrahydrofuranyl or tetrahydropyranyl;
(86) R₂ is selected from:
   (i) —C(O)NHR₂ₐ or —C(O)OR₂ₐ, wherein R₂ₐ is (1-2C)alkyl optionally substituted by hydroxy, amino or carboxyl;
   (ii) —N(H)C(O) R_{2c}, wherein R_{2c} is (1-2C)alkyl optionally substituted by halo, hydroxy, amino or carboxyl;
   (iii) —CN;
   (iv) a 5- or 6-membered heteroaryl comprising two, three or four nitrogen atoms;
   (v) oxetanyl, tetrahydrofuranyl or tetrahydropyranyl;
(87) R₂ is selected from:
   (i) —C(O)NHMe or —C(O)OMe;
   (ii) —N(H)C(O) R_{2c}, wherein R_{2c} is (1-2C)alkyl;
   (iii) —CN;
   (iv) a 5- or 6-membered heteroaryl comprising two, three or four nitrogen atoms;
(88) R₂ is selected from:
   (v) —N(H)C(O)R_{2c}, wherein R_{2c} is (1-2C)alkyl;
   (vi) —CN;
   (vii) a 5-membered heteroaryl comprising three or four nitrogen atoms;
(89) R₂ is selected from —N(H)C(O) Me, —CN or tetrazole;
(90) R₃ is selected from methyl, fluoro or chloro and integer b is 0;
(91) R₃ is selected from methyl, fluoro or chloro and integer b is 1;
(92) R₄ is a group R₁ as defined above;
(93) R₄ is a group R₂ as defined above;
(94) X₁ is CH or N;
   X₂ is CH or N;
   X₃ is CH or N;
   X₄ is CH or N;
   X₅/X₁₀/X₁₁ are each independently CH or N;
   X₁₂, when present, is CH or C(O);
with the proviso that:
   (i) only one or two of X₅/X₁₀/X₁₁ can be N;
   (ii) one, two or three of X₁, X₂, X₃, X₄ or X₅/X₁₀/X₁₁ is/are N; and
   (iii) if X₂ is N and R₄ is a R₁ group, then either at least one of X₁, X₃ or X₄ is N, and/or one or two of X₅/X₁₀/X₁₁ is N, and/or integer b is 1;
(95) X₁ is CH or N;
   X₂ is N;
   X₃ is CH or N;
   X₄ is CH or N;
   X₅/X₁₀/X₁₁ are each independently CH or N;
   X₁₂, when present, is CH or C(O);
with the proviso that:
   (i) only one or two of X₅/X₁₀/X₁₁ can be N;
   (ii) one, two or three of X₁, X₂, X₃, X₄ or X₅/X₁₀/X₁₁ is/are N; and
   (iii) if X₂ is N and R₄ is a R₁ group, then either at least one of X₁, X₃ or X₄ is N, and/or one or two of X₅/X₁₀/X₁₁ is N, and/or integer b is 1;
(96) X₁ is CH or N;
   X₂ is N;
   X₃ is CH or N;
   X₄ is CH or N;
   X₅ CH or N;
   X₁₀ and X₁₁ are both CH;
   X₁₂, when present, is CH or C(O);
with the proviso that:
   (i) one, two or three of X₁, X₂, X₃, X₄ or X₅ is/are N; and
   (ii) if X₂ is N and R₄ is a R₁ group, then either at least one of X₁, X₃ or X₄ is N, and/or X₅ is N, and/or integer b is 1;
(97) X₁ is CH;
   X₂ is N;
   X₃ is CH;
   X₄ is CH;
   X₅ CH or N;
   X₁₀ and X₁₁ are both CH;
   X₁₂, when present, is C(O);
with the proviso that:
   (i) one or two of X₁, X₂, X₃, X₄ or X₅ is/are N; and
   (ii) if R₄ is a R₁ group, then either X₅ is N, and/or integer b is 1;
(98) X₁ is CH;
   X₂ is N;
   X₃ is CH;
   X₄ is CH;
   X₅ CH;
   X₁₀ and X₁₁ are both CH;
   X₁₂, when present, is C(O);
with the proviso that:
   (i) if R₄ is a R₁ group, then integer b is 1;
(99) X₅/X₁₀/X₁₁ are each independently CH or N;
   X₆ is CH, NH, NMe or N;
   X₇ is CH, NH, NMe or N;
   X₈ is CH, NH, NMe or N;
   X₉ is C or N
   X₁₂, when present, is CH or C(O);
with the proviso that only one or two of X₅/X₁₀/X₁₁ can be N; and
only one of X₆, X₇ or X₈ can be NH or NMe;
(100) X₅/X₁₀/X₁₁ are each independently CH or N;
   X₆ is CH, NH or N;
   X₇ is CH, NH or N;
   X₈ is CH, NH or N;
   X₉ is C or N
   X₁₂, when present, is CH or C(O);
with the proviso that only one or two of X₅/X₁₀/X₁₁ can be N; and
only one of X₆, X₇ or X₈ can be NH;
(101) X₅ is CH;
   X₆ is CH, NH or N;
   X₇ is CH, NH or N;
   X₈ is CH, NH or N;
   X₉ is C or N;
   X₁₀ and X₁₁ are both CH;
   X₁₂, when present, is CH or C(O);
with the proviso that only one of X₆, X₇ or X₈ can be NH;
(102) X₅ is CH;
   X₆ is CH;
   X₇ is NH or N;
   X₈ is CH, NH or N;
   X₉ is C or N;
   X₁₀ and X₁₁ are both CH;
   X₁₂, when present, is CH or C(O);
with the proviso that only one of X₇ or X₈ can be NH;
(103) X₅ is CH;
   X₆ is CH;
   X₇ is N;
   X₈ is NH;
   X₉ is C;
   X₁₀ and X₁₁ are both CH;
   X₁₂, when present, is C(O);
(104) X₅ is CH;
   X₆ is CH;
   X₇ is N;
   X₈ is CH;
   X₉ is N;
   X₁₀ and X₁₁ are both CH;
   X₁₂, when present, is CH;
(105) X₅ is CH;
   X₆ is CH;
   X₇ is NH;
   X₈ is N;
   X₉ is C;
   X₁₀ and X₁₁ are both CH;
   X₁₂, when present, is C(O);
(106) R₅ is hydrogen or (1-2C)alkyl;
(107) R₅ is hydrogen or methyl;
(108) R₅ is hydrogen.

Suitably, in any of the definitions of formula I set out herein, at least one of Rₐ, R_{b}, R_{c}, R_{d} or Rₑ is a non-hydrogen substituent. By “non-hydrogen substituent” we mean a substituent selected from any one of the options defined herein for Rₐ, R_{b}, R_{c}, R_{d} or Rₑ other than hydrogen. More suitably, one to four of Rₐ, R_{b}, R_{c}, R_{d} or Rₑ is/are a non-hydrogen substituent(s). Most suitably, one to three of Rₐ, R_{b}, R_{c}, R_{d} or Rₑ is/are a non-hydrogen substituent(s).

Suitably, in any of the definitions of formula I set out herein, up to four of Rₐ, R_{b}, R_{c}, R_{d} or Rₑ are hydrogen and the remainder are non-hydrogen substituents (i.e. selected from any one of the options set out herein for Rₐ, R_{b}, R_{c}, R_{d} or Rₑ other than hydrogen). More suitably, two to four of Rₐ, R_{b}, R_{c}, R_{d} or Rₑ are hydrogen and the remainder are non-hydrogen substituents.

In a particular group of compounds of formula I, if R_{c} is a group of the formula —Y₂—[CH₂]₀₋₃—Z₂, then R_{b} and R_{d} cannot be a group of the formula —Y₁—[CH₂]₀₋₃—Z₁.

In a further group of compounds of formula I, if one or both of R_{b} and R_{d} is a group of the formula —Y₁—[CH₂]₀₋₃—Z₁ as defined herein, then R_{c} cannot be a group of the formula —Y₂—[CH₂]₀₋₃—Z₂.

Suitably, when R_{R} is a group of the formula Iq or Ir, L is a group of the formula:

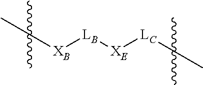

wherein:
X_{B}, L_{B}, L_{C} and X_{E} are each as defined above; or
   L is linker selected from:

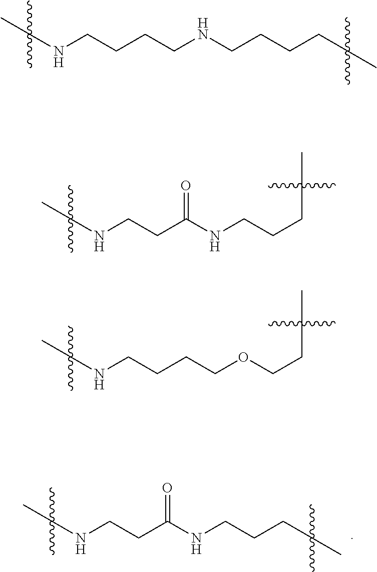 [SMILES: *CCCCNCCCCN* ; *CCCNC(=O)CCN* ; *CCOCCCCN* ; *CCCNC(=O)CCN*]

In a particular group of compounds of formula I:
(i) if R_{c} is a group of the formula —Y₂—[CH₂]₀₋₃—Z₂ then R_{b} and R_{d} cannot be a group of the formula —Y₁—[CH₂]₀₋₃—Z₁; and/or
(ii) if one or both of R_{b} and R_{d} is a group of the formula —Y₁—[CH₂]₀₋₃—Z₁ as defined herein, then R_{c} cannot be a group of the formula —Y₂—[CH₂]₀₋₃—Z₂.

In another particular group of compounds of formula I:
(i) if R_{c} is a group of the formula —Y₂—[CH₂]₀₋₃—Z₂ then R_{b} and R_{d} cannot be a group of the formula —Y₁—[CH₂]₀₋₃—Z₁; and
(ii) if one of R_{b} and R_{d} is a group of the formula —Y₁—[CH₂]₀₋₃—Z₁ as defined herein, then the other cannot be a group of the formula —Y₁—[CH₂]₀₋₃—Z₁ and R_{c} cannot be a group of the formula —Y₂—[CH₂]₀₋₃—Z₂.

Suitably, in any of the definitions of formula I set out herein, a heteroaryl is a 5- or 6-membered heteroaryl ring comprising one, two or three heteroatoms selected from N, O or S, unless specified otherwise.

Suitably, in any of the definitions of formula I set out herein, a heterocyclyl group is a 4-, 5- or 6-membered heterocyclyl ring comprising one, two or three heteroatoms selected from N, O or S, unless specified otherwise. Most suitably, a heterocyclyl group is a 4-, 5- or 6-membered ring comprising one or two heteroatoms selected from N, O or S [e.g. morpholinyl (e.g. 4-morpholinyl), piperidinyl, piperazinyl or pyrrolidinyl].

Suitably, in any of the definitions of formula I set out herein, L is as defined in formula I above or as defined in any one of paragraphs (1) to (10) above. More suitably, L is as defined in any one of paragraphs (3) to (10) above. Even more suitably, L is as defined in any one of paragraphs (6) to (10) above. Most suitably, L is as defined in any one of paragraphs (7), (8), (9) or (10) above.

Suitably, in any of the definitions of formula I set out herein, R_{L} is as defined in any one of paragraphs (11) to (15) above. More suitably, R_{L} is as defined in any one of paragraphs (12) to (15) above. Most suitably, R_{L} is as defined in any one of paragraphs (13), (14) or (15) above.

Suitably, in any of the definitions of formula I set out herein, Rₐ and Rₑ are as defined in any one of paragraphs (16) to (19) above. More suitably, Rₐ and Rₑ are as defined in any one of paragraphs (17), (18) or (19) above. Even more suitably, Rₐ and Rₑ are as defined in any one of paragraphs (18) or (19) above. Most suitably, Rₐ and Rₑ are as defined in paragraph (19) above.

In a particular group of compounds of formula I, Rₐ and Rₑ are as defined in paragraph (17) above, and L and R_{L}, and any group associated therewith, are each as defined in formula I above.

In a particular group of compounds of formula I, Rₐ and Rₑ are as defined in paragraph (19) above, and L and R_{L}, and any group associated therewith, are each as defined in formula I above.

Suitably, in any of the definitions of formula I set out herein, R_{b} and R_{d} are as defined in any one of paragraphs (20) to (35) above. More suitably, R_{b} and R_{d} are as defined in any one of paragraphs (25) to (35) above. Even more suitably, R_{b} and R_{d} are as defined in any one of paragraphs (31) to (35) above. Most suitably, R_{b} and R_{d} are as defined in any one of paragraphs (32), (33), (34) or (35) above.

In a particular group of compounds of formula I, R_{b} and R_{d} are as defined in paragraph (25) above, and L and R_{L}, and any group associated therewith, are each as defined in formula I above.

In a particular group of compounds of formula I, R_{b} and R_{d} are as defined in paragraph (31) above, and L and R_{L}, and any group associated therewith, are each as defined in formula I above.

In a particular group of compounds of formula I, R_{b} and R_{d} are as defined in paragraph (33) above, and L and R_{L}, and any group associated therewith, are each as defined in formula I above.

In a particular group of compounds of formula I, R_{b} and R_{d} are as defined in paragraph (34) above, and L and R_{L}, and any group associated therewith, are each as defined in formula I above.

In a particular group of compounds of formula I, R_{b} and R_{d} are as defined in paragraph (35) above, and L and R_{L}, and any group associated therewith, are each as defined in formula I above.

Suitably, in any of the definitions of formula I set out herein, R_{c} is as defined in any one of paragraphs (36) to (49) above. More suitably, R_{c} is as defined in any one of paragraphs (41) to (49) above. Even more suitably, R_{c} is as defined in any one of paragraphs (43) to (49) above. Yet even more suitable, R_{c} is as defined in any one of paragraphs (45) to (49) above. Most suitably, R_{c} is as defined in paragraphs (46), (47), (48) or (49) above.

In a particular group of compounds of formula I, R_{c} is as defined in paragraph (41) above, and L and R_{L}, and any group associated therewith, are each as defined in formula I above.

In a particular group of compounds of formula I, R_{c} is as defined in paragraph (43) above, and L and R_{L}, and any group associated therewith, are each as defined in formula I above.

In a particular group of compounds of formula I, R_{c} is as defined in paragraph (45) above, and L and R_{L}, and any group associated therewith, are each as defined in formula I above.

In a particular group of compounds of formula I, R_{c} is as defined in paragraph (47) above, and L and R_{L}, and any group associated therewith, are each as defined in formula I above.

In a particular group of compounds of formula I, R_{c} is as defined in paragraph (48) above, and L and R_{L}, and any group associated therewith, are each as defined in formula I above.

In a particular group of compounds of formula I, R_{c} is as defined in paragraph (49) above, and L and R_{L}, and any group associated therewith, are each as defined in formula I above.

In a particular group of compounds of formula I defined herein:
L is as defined in any one of paragraphs (1) to (10) above;
R_{L} is as defined in any one of paragraphs (11) to (15) above;
Rₐ and Rₑ are both as defined in paragraph (16) above;
R_{b} and R_{d} are both as defined in any one of paragraphs (20) to (35) above; and
R_{c} is as defined in any one of paragraphs (36) to (49) above.

In a particular group of compounds of formula I defined herein:
L is as defined in any one of paragraphs (1) to (10) above;
R_{L} is as defined in any one of paragraphs (11) to (15) above;
Rₐ and Rₑ are both as defined in paragraph (18) above;
R_{b} and R_{d} are both as defined in any one of paragraphs (20) to (35) above; and
R_{c} is as defined in any one of paragraphs (36) to (49) above.

In a particular group of compounds of formula I defined herein:
L is as defined in any one of paragraphs (1) to (10) above;
R_{L} is as defined in any one of paragraphs (11) to (15) above;
Rₐ and Rₑ are both as defined in paragraph (19) above;
R_{b} and R_{d} are both as defined in any one of paragraphs (20) to (35) above; and
R_{c} is as defined in any one of paragraphs (36) to (49) above.

In a particular group of compounds of formula I defined herein:
L is as defined in any one of paragraphs (1) to (10) above;
R_{L} is as defined in any one of paragraphs (11) to (15) above;
Rₐ and Rₑ are both as defined in paragraph (19) above;
R_{b} and R_{d} are both as defined in any one of paragraphs (26) to (35) above; and
R_{c} is as defined in any one of paragraphs (36) to (49) above.

In a particular group of compounds of formula I defined herein:
L is as defined in any one of paragraphs (1) to (10) above;
R_{L} is as defined in any one of paragraphs (11) to (15) above;
Rₐ and Rₑ are both as defined in paragraph (19) above;
R_{b} and R_{d} are both as defined in any one of paragraphs (31) to (35) above; and
R_{c} is as defined in any one of paragraphs (36) to (49) above.

In a particular group of compounds of formula I defined herein:
L is as defined in any one of paragraphs (1) to (10) above;
R_{L} is as defined in any one of paragraphs (11) to (15) above;
Rₐ and Rₑ are both as defined in paragraph (19) above;
R_{b} and R_{d} are both as defined in any one of paragraphs (32) to (35) above; and
R_{c} is as defined in any one of paragraphs (36) to (49) above.

In a particular group of compounds of formula I defined herein:
L is as defined in any one of paragraphs (1) to (10) above;
R_{L} is as defined in any one of paragraphs (11) to (15) above;
Rₐ and Rₑ are both as defined in paragraph (19) above;
R_{b} and R_{d} are both as defined in paragraph (33) above; and
R_{c} is as defined in any one of paragraphs (36) to (49) above.

In a particular group of compounds of formula I defined herein:
L is as defined in any one of paragraphs (1) to (10) above;
R_{L} is as defined in any one of paragraphs (11) to (15) above;
Rₐ and Rₑ are both as defined in paragraph (19) above;
R_{b} and R_{d} are both as defined in paragraph (34) above; and
R_{c} is as defined in any one of paragraphs (36) to (49) above.

In a particular group of compounds of formula I defined herein:
L is as defined in any one of paragraphs (1) to (10) above;
R_{L} is as defined in any one of paragraphs (11) to (15) above;
Rₐ and Rₑ are both as defined in paragraph (19) above;
R_{b} and R_{d} are both as defined in paragraph (35) above; and
R_{c} is as defined in any one of paragraphs (36) to (49) above.

In a particular group of compounds of formula I defined herein:
L is as defined in any one of paragraphs (1) to (10) above;
R_{L} is as defined in any one of paragraphs (11) to (15) above;
Rₐ and Rₑ are both as defined in paragraph (19) above;
R_{b} and R_{d} are both as defined in any one of paragraphs (20) to (35) above; and
R_{c} is as defined in any one of paragraphs (41) to (49) above.

In a particular group of compounds of formula I defined herein:
L is as defined in any one of paragraphs (1) to (10) above;
R_{L} is as defined in any one of paragraphs (11) to (15) above;
Rₐ and Rₑ are both as defined in paragraph (19) above;
R_{b} and R_{d} are both as defined in any one of paragraphs (20) to (35) above; and
R_{c} is as defined in any one of paragraphs (43) to (49) above.

In a particular group of compounds of formula I defined herein:
L is as defined in any one of paragraphs (1) to (10) above;
R_{L} is as defined in any one of paragraphs (11) to (15) above;
Rₐ and Rₑ are both as defined in paragraph (19) above;
R_{b} and R_{d} are both as defined in any one of paragraphs (20) to (35) above; and
R_{c} is as defined in any one of paragraphs (45) to (49) above.

In a particular group of compounds of formula I defined herein:
L is as defined in any one of paragraphs (1) to (10) above;
R_{L} is as defined in any one of paragraphs (11) to (15) above;
Rₐ and Rₑ are both as defined in paragraph (19) above;
R_{b} and R_{d} are both as defined in any one of paragraphs (20) to (35) above; and
R_{c} is as defined in paragraph (47) above.

In a particular group of compounds of formula I defined herein:
L is as defined in any one of paragraphs (1) to (10) above;
R_{L} is as defined in any one of paragraphs (11) to (15) above;
Rₐ and Rₑ are both as defined in paragraph (19) above;
R_{b} and R_{d} are both as defined in any one of paragraphs (20) to (35) above; and
R_{c} is as defined in paragraph (48).

In a particular group of compounds of formula I defined herein:
L is as defined in any one of paragraphs (1) to (10) above;
R_{L} is as defined in any one of paragraphs (11) to (15) above;
Rₐ and Rₑ are both as defined in paragraph (19) above;
R_{b} and R_{d} are both as defined in any one of paragraphs (20) to (35) above; and
R_{c} is as defined in paragraph (49).

In a particular group of compounds of formula I defined herein:
L is as defined in any one of paragraphs (1) to (10) above;
R_{L} is as defined in any one of paragraphs (11) to (15) above;
Rₐ and Rₑ are both as defined in paragraph (19) above;
R_{b} and R_{d} are both as defined in any one of paragraphs (26) to (35) above; and
R_{c} is as defined in any one of paragraphs (41) to (49) above.

In a particular group of compounds of formula I defined herein:
L is as defined in any one of paragraphs (1) to (10) above;
R_{L} is as defined in any one of paragraphs (11) to (15) above;
Rₐ and Rₑ are both as defined in paragraph (19) above;
R_{b} and R_{d} are both as defined in any one of paragraphs (31) to (35) above; and
R_{c} is as defined in any one of paragraphs (42) to (49) above.

In a particular group of compounds of formula I defined herein:
L is as defined in any one of paragraphs (1) to (10) above;
R_{L} is as defined in any one of paragraphs (11) to (15) above;
Rₐ and Rₑ are both as defined in paragraph (19) above;
R_{b} and R_{d} are both as defined in any one of paragraphs (32) to (35) above; and
R_{c} is as defined in any one of paragraphs (45) to (49) above.

In a particular group of compounds of formula I defined herein:
L is as defined in any one of paragraphs (1) to (10) above;
R_{L} is as defined in any one of paragraphs (11) to (15) above;
Rₐ and Rₑ are both as defined in paragraph (19) above;
R_{b} and R_{d} are both as defined in paragraph (33) above; and
R_{c} is as defined in paragraph (47) above.

In a particular group of compounds of formula I defined herein:
L is as defined in any one of paragraphs (1) to (10) above;
R_{L} is as defined in any one of paragraphs (11) to (15) above;
Rₐ and Rₑ are both as defined in paragraph (19) above;
R_{b} and R_{d} are both as defined in paragraph (34) above; and
R_{c} is as defined in paragraph (48) above.

In a particular group of compounds of formula I defined herein:
L is as defined in any one of paragraphs (1) to (10) above;
R_{L} is as defined in any one of paragraphs (11) to (15) above;
Rₐ and Rₑ are both as defined in paragraph (19) above;
R_{b} and R_{d} are both as defined in paragraph (35) above; and
R_{c} is as defined in paragraph (49) above.

In a particular group of compounds of formula I defined herein:
L is as defined in any one of paragraphs (3) to (10) above;
R_{L} is as defined in any one of paragraphs (11) to (15) above;
Rₐ and Rₑ are both as defined in paragraph (19) above;
R_{b} and R_{d} are both as defined in any one of paragraphs (26) to (35) above; and
R_{c} is as defined in any one of paragraphs (41) to (49) above.

In a particular group of compounds of formula I defined herein:
L is as defined in any one of paragraphs (5) to (10) above;
R_{L} is as defined in any one of paragraphs (11) to (15) above;
Rₐ and Rₑ are both as defined in paragraph (19) above;
R_{b} and R_{d} are both as defined in any one of paragraphs (31) to (35) above; and
R_{c} is as defined in any one of paragraphs (43) to (49) above.

In a particular group of compounds of formula I defined herein:
L is as defined in any paragraph (6) above;
R_{L} is as defined in any one of paragraphs (11) to (15) above;
Rₐ and Rₑ are both as defined in paragraph (19) above;
R_{b} and R_{d} are both as defined in any one of paragraphs (32) to (35) above; and
R_{c} is as defined in any one of paragraphs (45) to (49) above.

In a particular group of compounds of formula I defined herein:
L is as defined in paragraph (7) above;
R_{L} is as defined in any one of paragraphs (11) to (15) above;
Rₐ and Rₑ are both as defined in paragraph (19) above;
R_{b} and R_{d} are both as defined in paragraph (33) above; and
R_{c} is as defined in paragraph (47) above.

In a particular group of compounds of formula I defined herein:
L is as defined in paragraph (8) above;
R_{L} is as defined in any one of paragraphs (11) to (15) above;
Rₐ and Rₑ are both as defined in paragraph (19) above;
R_{b} and R_{d} are both as defined in paragraph (34) above; and
R_{c} is as defined in paragraph (46) above.

In a particular group of compounds of formula I defined herein:
L is as defined in paragraph (9) above;
R_{L} is as defined in any one of paragraphs (11) to (15) above;
Rₐ and Rₑ are both as defined in paragraph (19) above;
R_{b} and R_{d} are both as defined in paragraph (35) above; and
R_{c} is as defined in paragraph (49) above.

In a particular group of compounds of formula I defined herein:
L is as defined in paragraph (10) above;
R_{L} is as defined in any one of paragraphs (11) to (15) above;
Rₐ and Rₑ are both as defined in paragraph (19) above;
R_{b} and R_{d} are both as defined in paragraph (35) above; and
R_{c} is as defined in paragraph (49) above.

Suitably, in any of the definitions of formula I set out herein, R₁₀₀ is as defined in formula I above, or as defined in any one of paragraphs (50) or (51) above.

Suitably, in any of the definitions of formula I set out herein, R₁₀₀ₐ is as defined in formula I above, or as defined in any one of paragraphs (52) or (53) above.

Suitably, in any of the definitions of formula I set out herein, R₁₀₁ is as defined in formula I above, or as defined in any one of paragraphs (56) or (57) above.

Suitably, in any of the definitions of formula I set out herein, R₁₀₁ₐ is as defined in formula I above, or as defined in paragraph (58) above.

Suitably, in any of the definitions of formula I set out herein, integer a is as defined in formula I above, or as defined in paragraph (61) above.

Suitably, in any of the definitions of formula I set out herein, Q₁ is as defined in formula I above, or as defined in any one of paragraphs (62) or (63) above.

Suitably, in any of the definitions of formula I set out herein, Q₂, Q₃, Q₄, Q₅ and Q₆ is as defined in formula I above, or as defined in any one of paragraphs (65) to (70) above. More, suitably, Q₂, Q₃, Q₄, Q₅ and Q₆ is as defined in formula I above, or as defined in any one of paragraphs (66) to (69) above.

Suitably, in any of the definitions of formula I set out herein, Q₇, Q₈, Q₉ and Q₁₀ is as defined in formula I above, or as defined in any one of paragraphs (71) to (73) above. More suitably, in any of the definitions of formula I set out herein, Q₇, Q₈, Q₉ and Q₁₀ is as defined in formula I above, or as defined in any one of paragraphs (72) or (73) above.

Suitably, in any of the definitions of formula I set out herein, ring A is as defined in formula I above, or as defined in any one of paragraphs (74) to (78) above. More suitably, ring A is as defined in any one of paragraphs (76), (77) or (78) above.

Suitably, in any of the definitions of formula I set out herein, R_{R} is as defined in formula I above or as defined in any one of paragraphs (79) to (82) above.

Suitably, in any of the definitions of formula I set out herein, R₁ is as defined in paragraph (83) or (84) above.

Suitably, in any of the definitions of formula I set out herein, R₂ is as defined in any one of paragraphs (85) to (89) above. More suitably, R₂ is as defined in any one of paragraphs (87) to (89) above. Even more suitably, R₂ is as defined in any one of paragraphs (88) or (89) above.

Suitably, in any of the definitions of formula I set out herein, R₃ is as defined in any one of paragraphs (90) or (91) above. More suitably, R₃ is as defined in paragraph (90) above.

Suitably, in any of the definitions of formula I set out herein, R₄ is as defined in any one of paragraphs (92) or (93) above. More suitably, R₄ is as defined in paragraph (92) above.

Suitably, in any of the definitions of formula I set out herein, X₁, X₂, X₃, X₄ and X₅/X₁₀/X₁₁ are as defined in any one of paragraphs (94) to (98) above. More suitably, X₁, X₂, X₃, X₄ and X₅/X₁₀/X₁₁ are as defined in any one of paragraphs (95) to (98) above. Even more suitably, X₁, X₂, X₃, X₄ and X₅/X₁₀/X₁₁ are as defined in any one of paragraphs (96), (97) or (98) above.

Suitably, in any of the definitions of formula I set out herein, X₆, X₇, X₈, X₉ and X₅/X₁₀/X₁₁ are as defined in any one of paragraphs (99) to (105) above. More suitably, X₆, X₇, X₈, X₉ and X₅/X₁₀/X₁₁ are as defined in any one of paragraphs (101) to (105) above. Even more suitably, X₆, X₇, X₈, X₉ and X₅/X₁₀/X₁₁ are as defined in any one of paragraphs (102) to (105) above. Yet even more suitably, X₆, X₇, X₈, X₉ and X₅/X₁₀/X₁₁ are as defined in any one of paragraphs (103), (104) or (105) above.

Suitably, in any of the definitions of formula I set out herein, R₅ is as defined in any one of paragraphs (106), (107) or (108) above. More suitably, R₅ is as defined in paragraph (108) above.

In a particular group of compounds of formula I, R_{R} is as defined in paragraph (79) above, and L and R_{L}, and any group associated therewith, are each as defined in formula I above.

In a particular group of compounds of formula I, R_{R} is as defined in paragraph (80) above, and L and R_{L}, and any group associated therewith, are each as defined in formula I above.

In a particular group of compounds of formula I, R_{R} is as defined in paragraph (81) above, and L and R_{L}, and any group associated therewith, are each as defined in formula I above.

In a particular group of compounds of formula I, R_{R} is as defined in paragraph (82) above, and L and R_{L}, and any group associated therewith, are each as defined in formula I above.

In a particular group of compounds of formula I, R_{R} is as defined in any one of paragraphs (79) to (82) above, R₁ is as defined in paragraph (83) or (84) above or R₂ is as defined in any one of paragraphs (85) to (89) above, and L and R_{L}, and any group associated therewith, are each as defined in formula I above. More suitably, R_{R} is as defined in any one of paragraphs (79) to (82) above, R₁ is as defined in paragraph (83) or (84) above or R₂ is as defined in paragraph (89) above, and L and R_{L}, and any group associated therewith, are each as defined in formula I above.

In a particular group of compounds of formula I, R_{R} is as defined in any one of paragraphs (80) to (82) above, R₃ is as defined in paragraph (90) or (91) above or R₄ is as defined in paragraph (92) or (93) above, and L and R_{L}, and any group associated therewith, are each as defined in formula I above. More suitably, R_{R} is as defined in any one of paragraphs (80) to (82) above, R₃ is as defined in paragraph (90) above or R₃ is as defined in paragraph (92) above, and L and R_{L}, and any group associated therewith, are each as defined in formula I above.

In a particular group of compounds of formula I, R_{R} is as defined in any one of paragraphs (80) to (82) above, R₄ is as defined in paragraph (92) or (93) above and/or R₅ is as defined in any one of paragraphs (106) to (108), and L and R_{L}, and any group associated therewith, are each as defined in formula I above. More suitably, R_{R} is as defined in any one of paragraphs (80) to (82) above, R₄ is as defined in paragraph (92) above and/or R₅ is as defined in paragraph (108) above, and L and R_{L}, and any group associated therewith, are each as defined in formula I above.

In a particular group of compounds of the invention, the compound is a compound of formula I defined herein in which R_{L} is as defined in paragraph (11) above and Rₐ and Rₑ are as defined in paragraph (19) above, i.e. the compounds have the formula Is shown below, or a pharmaceutically acceptable salt thereof:

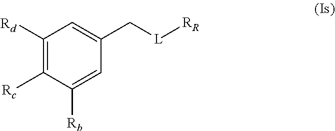

wherein L, R_{R}, R_{b}, R_{c} and R_{d}, and any groups associated therewith, each have any one of the definitions set out hereinbefore.

In a particular group of compounds of formula Is:
L is as defined in any one of paragraphs (1) to (10) above;
R_{R} is as defined in any one of paragraphs (79) to (82) above;
R_{b} and R_{d} are both as defined in any one of paragraphs (20) to (35) above;
R_{c} is as defined in any one of paragraphs (37) to (49) above.

In a particular group of compounds of formula Is:
L is as defined in any one of paragraphs (3) to (10) above;
R_{R} is as defined in any one of paragraphs (79) to (82) above;
R_{b} and R_{d} are both as defined in any one of paragraphs (25) to (35) above;
R_{c} is as defined in any one of paragraphs (41) to (49) above.

In a particular group of compounds of formula Is:
L is as defined in any one of paragraphs (6) to (10) above;
R_{R} is as defined in any one of paragraphs (79) to (82) above;
R_{b} and R_{d} are both as defined in any one of paragraphs (31) to (35) above;
R_{c} is as defined in any one of paragraphs (45) to (49) above.

In a particular group of compounds of formula Is:
L is as defined in any one of paragraphs (7), (8), (9) or (10) above;
R_{R} is as defined in any one of paragraphs (79) to (82) above;
R_{b} and R_{d} are both as defined in any one of paragraphs (33), (34) or (35) above;
R_{c} is as defined in any one of paragraphs (47), (48) or (49) above.

In a particular group of compounds of formula Is:
L is as defined in paragraph (9) above;
R_{R} is as defined in any one of paragraphs (79) to (82) above;
R_{b} and R_{d} are both as defined in paragraph (35) above;
R_{c} is as defined in paragraph (49) above.

In a particular group of compounds of formula Is:
L is as defined in paragraph (10) above;
R_{R} is as defined in any one of paragraphs (79) to (82) above;
R_{b} and R_{d} are both as defined in paragraph (35) above;
R_{c} is as defined in paragraph (49) above.

In a particular group of compounds of the invention, the compound is a compound of formula I defined herein in which R_{L} is as defined in paragraph (10) above and Rₐ and Rₑ are as defined in paragraph (18) above, i.e. the compounds have the formula It shown below, or a pharmaceutically acceptable salt thereof:

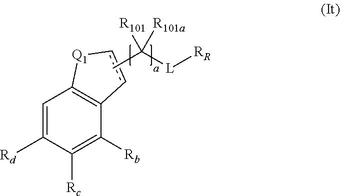

wherein L, R_{R}, a, R₁₀₁, R₁₀₁ₐ, Q₁, R_{b}, R_{c} and R_{d} each have any one of the definitions set out herein.

In a particular group of compounds of formula It:
L is as defined in any one of paragraphs (1) to (10) above;
R_{R} is as defined in any one of paragraphs (79) to (82) above;
Integer a is as defined in paragraph (61) above;
R₁₀₁ is as defined in any one of paragraphs (56) or (57) above;
R₁₀₁ₐ is as defined in paragraph (58) above;
Q₁ is as defined in any one of paragraphs (62) or (63) above;
R_{b} and R_{d} are both as defined in any one of paragraphs (20) to (35) above;
R_{c} is as defined in any one of paragraphs (36) to (49) above.

In a particular group of compounds of formula It:
L is as defined in any one of paragraphs (3) to (10) above;
R_{R} is as defined in any one of paragraphs (79) to (82) above;
Integer a is as defined in paragraph (61) above;
R₁₀₁ is as defined in any one of paragraphs (56) or (57) above;
R₁₀₁ₐ is as defined in paragraph (58) above;
Q₁ is as defined in any one of paragraphs (62) or (63) above;
R_{b} and R_{d} are both as defined in any one of paragraphs (25) to (35) above;
R_{c} is as defined in any one of paragraphs (41) to (49) above.

In a particular group of compounds of formula It:
L is as defined in any one of paragraphs (6) to (10) above;
R_{R} is as defined in any one of paragraphs (79) to (82) above;
Integer a is as defined in paragraph (61) above;
R₁₀₁ is as defined in any one of paragraphs (56) or (57) above;
R₁₀₁ₐ is as defined in paragraph (58) above;
Q₁ is as defined in any one of paragraphs (62) or (63) above;
R_{b} and R_{d} are both as defined in any one of paragraphs (31) to (35) above;
R_{c} is as defined in any one of paragraphs (45) to (49) above.

In a particular group of compounds of formula It:
L is as defined in any one of paragraphs (7), (8), (9) or (10) above;
R_{R} is as defined in any one of paragraphs (79) to (82) above;
Integer a is as defined in paragraph (61) above;
R₁₀₁ is as defined in paragraph (57) above;
R₁₀₁ₐ is as defined in paragraph (58) above;
Q₁ is as defined in any one of paragraphs (62) or (63) above;
R_{b} and R_{d} are both as defined in any one of paragraphs (33), (34) or (35) above;
R_{c} is as defined in any one of paragraphs (47), (48) or (49) above.

In a particular group of compounds of formula It:
L is as defined in paragraph (10) above;
R_{R} is as defined in any one of paragraphs (79) to (82) above;
Integer a is as defined in paragraph (61) above;
R₁₀₁ is as defined in paragraph (57) above;
R₁₀₁ₐ is as defined in paragraph (58) above;
Q₁ is as defined in paragraph (63) above;
R_{b} and R_{d} are both as defined in paragraph (35) above;
R_{c} is as defined in paragraph (49) above.

In a particular group of compounds of formula It:
L is as defined in paragraph (10) above;
R_{R} is as defined in any one of paragraphs (79) to (82) above;
Integer a is as defined in paragraph (61) above;
R₁₀₁ is as defined in paragraph (57) above;
R₁₀₁ₐ is as defined in paragraph (58) above;
Q₁ is as defined in paragraph (63) above;
R_{b} and R_{d} are both as defined in paragraph (35) above;
R_{c} is as defined in paragraph (49) above.

In a particular group of compounds of the invention, the compound is a compound of formula I defined herein in which R_{L} is as defined in paragraph (11) above, i.e. the compounds have the formula Iu shown below, or a pharmaceutically acceptable salt thereof:

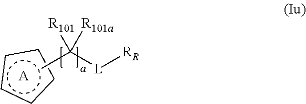

wherein L, R_{R}, a, R₁₀₁, R₁₀₁ₐ and ring A each have any one of the definitions set out herein.

In a particular group of compounds of formula Iu:
L is as defined in any one of paragraphs (1) to (10) above;
R_{R} is as defined in any one of paragraphs (79) to (82) above;
Integer a is as defined in paragraph (61) above;
R₁₀₁ is as defined in any one of paragraphs (56) or (57) above;
R₁₀₁ₐ is as defined in paragraph (58) above;
Ring A is as defined in any one of paragraphs (74) to (78) above.

In a particular group of compounds of formula Iu:
L is as defined in any one of paragraphs (3) to (10) above;
R_{R} is as defined in any one of paragraphs (79) to (82) above;
Integer a is as defined in paragraph (61) above;
R₁₀₁ is as defined in any one of paragraphs (56) or (57) above;
R₁₀₁ₐ is as defined in paragraph (58) above;
Ring A is as defined in any one of paragraphs (75) to (78) above.

In a particular group of compounds of formula Iu:
L is as defined in any one of paragraphs (6) to (10) above;
R_{R} is as defined in any one of paragraphs (79) to (82) above;
Integer a is as defined in paragraph (61) above;
R₁₀₁ is as defined in any one of paragraphs (56) or (57) above;
R₁₀₁ₐ is as defined in paragraph (58) above;
Ring A is as defined in any one of paragraphs (76), (77) or (78) above.

In a particular group of compounds of formula Iu:
L is as defined in any one of paragraphs (7), (8), (9) or (10) above;
R_{R} is as defined in any one of paragraphs (79) to (82) above;
Integer a is as defined in paragraph (61) above;
R₁₀₁ is as defined in paragraph (57) above;
R₁₀₁ₐ is as defined in paragraph (58) above;
Ring A is as defined in paragraphs (77) or (78) above.

In a particular group of compounds of formula Iu:
L is as defined in paragraph (9) above;
R_{R} is as defined in any one of paragraphs (79) to (82) above;
Integer a is as defined in paragraph (61) above;
R₁₀₁ is as defined in paragraph (57) above;
R₁₀₁ₐ is as defined in paragraph (58) above;
Ring A is as defined in paragraph (78) above.

In a particular group of compounds of formula Iu:
L is as defined in paragraph (10) above;
R_{R} is as defined in any one of paragraphs (79) to (82) above;
Integer a is as defined in paragraph (61) above;
R₁₀₁ is as defined in paragraph (57) above;
R₁₀₁ₐ is as defined in paragraph (58) above;
Ring A is as defined in paragraph (78) above.

In a particular group of compounds of the invention, the compound is a compound of formula I defined herein in which R_{R} is as defined in paragraph (79) above, i.e. the compounds have the formula Iv shown below, or a pharmaceutically acceptable salt thereof:

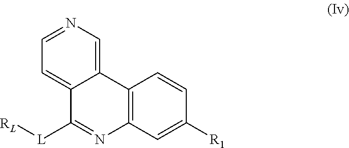

wherein L, R_{L} and R₁ each have any one of the definitions set out herein.

In a particular group of compounds of formula Iv:
L is as defined in any one of paragraphs (1) to (10) above;
R_{L} is as defined in any one of paragraphs (11) to (15) above;
R₁ is as defined in any one of paragraphs (83) or (84) above.

In a particular group of compounds of formula Iv:
L is as defined in any one of paragraphs (3) to (10) above;
R_{L} is as defined in any one of paragraphs (12) to (15) above;
R₁ is as defined in any one of paragraphs (83) or (84) above.

In a particular group of compounds of formula Iv:
L is as defined in any one of paragraphs (6) to (10) above;
R_{L} is as defined in any one of paragraphs (13) to (15) above;
R₁ is as defined in any one of paragraphs (83) or (84) above.

In a particular group of compounds of formula Iv:
L is as defined in any one of paragraphs (7), (8), (9) or (10) above;
R_{L} is as defined in any one of paragraphs (12) or (15) above;
R₁ is as defined in any one of paragraphs (83) or (84) above.

In a particular group of compounds of formula Iv:
L is as defined in paragraph (9) above;
R_{L} is as defined in any one of paragraphs (14) or (15) above;
R₁ is as defined in any one of paragraphs (83) or (84) above.

In a particular group of compounds of formula Iv:
L is as defined in paragraph (10) above;
R_{L} is as defined in any one of paragraphs (14) or (15) above;
R₁ is as defined in any one of paragraphs (83) or (84) above.

In a particular group of compounds of the invention, the compound is a compound of formula I defined herein in which R_{R} is as defined in paragraph (79) above, i.e. the compounds have the formula Iw shown below, or a pharmaceutically acceptable salt thereof:

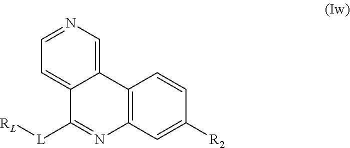

wherein L, R_{L} and R₂ each have any one of the definitions set out herein.

In a particular group of compounds of formula Iw:
L is as defined in any one of paragraphs (1) to (10) above;
R_{L} is as defined in any one of paragraphs (11) to (15) above;
R₂ is as defined in any one of paragraphs (85) to (88) above.

In a particular group of compounds of formula Iw:
L is as defined in any one of paragraphs (3) to (10) above;
R_{L} is as defined in any one of paragraphs (12) to (15) above;
R₂ is as defined in any one of paragraphs (86), (87) or (88) above.

In a particular group of compounds of formula Iw:
L is as defined in any one of paragraphs (6) to (10) above;
R_{L} is as defined in any one of paragraphs (13) to (15) above;
R₂ is as defined in any one of paragraphs (86), (87) or (88) above.

In a particular group of compounds of formula Iw:
L is as defined in any one of paragraphs (7), (8), (9) or (10) above;
R_{L} is as defined in any one of paragraphs (14) or (15) above;
R₂ is as defined in any one of paragraphs (87) or (88) above.

In a particular group of compounds of formula Iw:
L is as defined in paragraph (9) above;
R_{L} is as defined in any one of paragraphs (14) or (15) above;
R₂ is as defined in paragraph (88) above.

In a particular group of compounds of formula Iw:
L is as defined in paragraph (10) above;
R_{L} is as defined in any one of paragraphs (14) or (15) above;
R₂ is as defined in paragraph (88) above.

In a particular group of compounds of the invention, the compound is a compound of formula I defined herein in which R_{R} is as defined in paragraph (80) above, X₅, X₁₀ and X₁₁ are as defined in paragraph (105) and R₃ is as defined in paragraph (90), i.e. the compounds have the formula Ix shown below, or a pharmaceutically acceptable salt thereof:

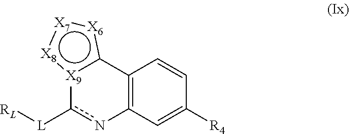

wherein L, R_{L}, R₄, X₆, X₇, X₈ and X₉ each have any one of the definitions set out herein.

In a particular group of compounds of formula Ix:
L is as defined in any one of paragraphs (1) to (10) above;
R_{L} is as defined in any one of paragraphs (11) to (15) above;
R₄ is as defined in any one of paragraphs (92) or (93) above;
X₆, X₇, X₈ and X₉ are as defined in any one of paragraphs (98) to (104) above.

In a particular group of compounds of formula Ix:
L is as defined in any one of paragraphs (3) to (10) above;
R_{L} is as defined in any one of paragraphs (12) to (15) above;
R₄ is as defined in any one of paragraphs (92) or (93) above;
X₆, X₇, X₈ and X₉ are as defined in any one of paragraphs (100) to (105) above.

In a particular group of compounds of formula Ix:
L is as defined in any one of paragraphs (6) to (10) above;
R_{L} is as defined in any one of paragraphs (13) to (15) above;
R₄ is as defined in any one of paragraphs (92) or (93) above;
X₆, X₇, X₈ and X₉ are as defined in any one of paragraphs (101) to (105) above.

In a particular group of compounds of formula Ix:
L is as defined in any one of paragraphs (7), (8), (9) or (10) above;
R_{L} is as defined in any one of paragraphs (14) or (15) above;
R₄ is as defined in paragraph (92) above;
X₆, X₇, X₈ and X₉ are as defined in any one of paragraphs (102) to (105) above.

In a particular group of compounds of formula Ix:
L is as defined in paragraph (9) above;
R_{L} is as defined in any one of paragraphs (14) or (15) above;
R₄ is as defined in paragraph (92) above;
X₆, X₇, X₈ and X₉ are as defined in any one of paragraphs (103), (104) or (105) above.

In a particular group of compounds of formula Ix:
L is as defined in paragraph (10) above;
R_{L} is as defined in any one of paragraphs (14) or (15) above;
R₄ is as defined in paragraph (92) above;
X₆, X₇, X₈ and X₉ are as defined in any one of paragraphs (103), (104) or (105) above.

In a particular group of compounds of the invention, the compound is a compound of formula I defined herein in which R_{R} is as defined in paragraph (81) above, X₅, X₁₀ and X₁₁ are as defined in paragraph (105) and R₃ is as defined in paragraph (90), i.e. the compounds have the formula Iy shown below, or a pharmaceutically acceptable salt thereof:

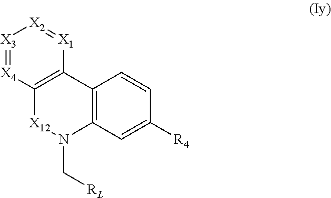

wherein L, R_{L}, R₄, X₁, X₂, X₃, X₄ and X₁₂ each have any one of the definitions set out herein.

In a particular group of compounds of formula Iy:
L is as defined in any one of paragraphs (1) to (10) above;
R_{L} is as defined in any one of paragraphs (11) to (15) above;
R₄ is as defined in any one of paragraphs (92) or (93) above;
X₁, X₂, X₃, X₄ and X₁₂ are as defined in any one of paragraphs (94) to (98) above.

In a particular group of compounds of formula Iy:
L is as defined in any one of paragraphs (3) to (10) above;
R_{L} is as defined in any one of paragraphs (12) to (15) above;
R₄ is as defined in any one of paragraphs (92) or (93) above;
X₁, X₂, X₃, X₄ and X₁₂ are as defined in any one of paragraphs (95) to (98) above.

In a particular group of compounds of formula Iy:
L is as defined in any one of paragraphs (6) to (10) above;
R_{L} is as defined in any one of paragraphs (13) to (15) above;
R₄ is as defined in any one of paragraphs (92) or (93) above;
X₁, X₂, X₃, X₄ and X₁₂ are as defined in any one of paragraphs (96), (97) or (98) above.

In a particular group of compounds of formula Iy:
L is as defined in any one of paragraphs (7), (8), (9) or (10) above;
R_{L} is as defined in any one of paragraphs (14) or (15) above;
R₄ is as defined in any one of paragraphs (92) or (93) above;
X₁, X₂, X₃, X₄ and X₁₂ are as defined in any one of paragraphs (97) or (98) above.

In a particular group of compounds of formula Iy:
L is as defined in paragraph (9) above;
R_{L} is as defined in any one of paragraphs (14) or (15) above;
R₄ is as defined in any paragraph (92) above;
X₁, X₂, X₃, X₄ and X₁₂ are as defined in paragraph (98) above.

In a particular group of compounds of formula Iy:
L is as defined in paragraph (10) above;
R_{L} is as defined in any one of paragraphs (14) or (15) above;
R₄ is as defined in any paragraph (92) above;
X₁, X₂, X₃, X₄ and X₁₂ are as defined in paragraph (98) above.

In a particular group of compounds of the invention, the compound is a compound of formula I defined herein in which R_{R} is as defined in paragraph (80) above, X₅, X₁₀ and X₁₁ are as defined in paragraph (104) and R₃ is as defined in paragraph (89), i.e. the compounds have the formula Iy shown below, or a pharmaceutically acceptable salt thereof:

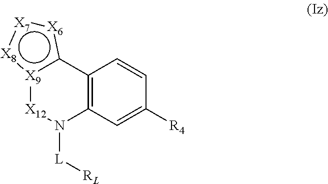

wherein L, R_{L}, R₄, X₆, X₇, X₈, X₉ and X₁₂ each have any one of the definitions set out herein.

In a particular group of compounds of formula Iz:
L is as defined in any one of paragraphs (1) to (10) above;
R_{L} is as defined in any one of paragraphs (11) to (15) above;
R₄ is as defined in any one of paragraphs (92) or (93) above;
X₆, X₇, X₈, X₉ and X₁₂ are as defined in any one of paragraphs (99) to (105) above.

In a particular group of compounds of formula Iz:
L is as defined in any one of paragraphs (3) to (10) above;
R_{L} is as defined in any one of paragraphs (12) to (15) above;
R₄ is as defined in any one of paragraphs (92) or (93) above;
X₆, X₇, X₈, X₉ and X₁₂ are as defined in any one of paragraphs (100) to (105) above.

In a particular group of compounds of formula Iz:
L is as defined in any one of paragraphs (6) to (10) above;
R_{L} is as defined in any one of paragraphs (13) to (15) above;
R₄ is as defined in any one of paragraphs (92) or (93) above;
X₆, X₇, X₈, X₉ and X₁₂ are as defined in any one of paragraphs (101) to (105) above.

In a particular group of compounds of formula Iz:
L is as defined in any one of paragraphs (7), (8), (9) or (10) above;
R_{L} is as defined in any one of paragraphs (14) or (15) above;
R₄ is as defined in any one of paragraphs (92) or (93) above;
X₆, X₇, X₈, X₉ and X₁₂ are as defined in any one of paragraphs (102) to (105) above.

In a particular group of compounds of formula Iz:
L is as defined in paragraph (9) above;
R_{L} is as defined in any one of paragraphs (14) or (15) above;
R₄ is as defined in paragraph (92) above;
X₆, X₇, X₈, X₉ and X₁₂ are as defined in paragraphs (103), (104) or (105) above.

In a particular group of compounds of formula Iz:
L is as defined in paragraph (10) above;
R_{L} is as defined in any one of paragraphs (14) or (15) above;
R₄ is as defined in paragraph (92) above;
X₆, X₇, X₈, X₉ and X₁₂ are as defined in paragraphs (103), (104) or (105) above.

Particular compounds of the present invention include any of the compounds described in the example section of the present application, or a pharmaceutically acceptable salt, hydrate or solvate thereof, and, in particular, any of the following:
5-[4-({4-[({2-chloro-[1,1′-biphenyl]-4-yl}methyl)amino]butyl}amino)butyl]benzo[c]2,6-naphthyridine-8-carboxylic acid;
5-{[2-({4-[({2-chloro-[1,1′-biphenyl]-4-yl}methyl)amino]butyl}amino)ethyl]amino}benzo[c]2,6-naphthyridine-8-carboxylic acid;
5-{[3-({4-[({2-chloro-[1,1′-biphenyl]-4-yl}methyl)amino]butyl}amino)propyl]amino}benzo[c]2,6-naphthyridine-8-carboxylic acid;
5-((2-((2-(((2-chloro-[1,1′-biphenyl]-4-yl)methyl)amino)ethyl)amino)ethyl)amino)benzo[c][2,6]naphthyridine-8-carboxylic acid;
5-(3-(3-(((2-Chloro-[1,1′-biphenyl]-4-yl)methyl)amino)propanamido)propyl)-4-oxo-4,5-dihydro-3H-pyrazolo[3,4-c]quinoline-7-carboxylic acid;
5-((2-(4-(((2-Chloro-[1,1′-biphenyl]-4-yl)methyl)amino)butanamido)ethyl)amino)benzo[c][2,6]naphthyridine-8-carboxylic acid;
5-((2-((4-(((2-chloro-[1,1′-biphenyl]-4-yl)methyl)amino)butyl)amino)ethyl)amino)benzo[c][2,6]naphthyridine-8-carboxamide;
5-((2-((4-((3-Chlorobenzyl)amino)butyl)amino)ethyl)amino)benzo[c][2,6]naphthyridine-8-carboxamide;
5-((2-((4-(((2-Chloro-2′-(hydroxymethyl)-[1,1′-biphenyl]-4-yl)methyl)amino)butyl)amino)ethyl)amino)benzo[c][2,6]naphthyridine-8-carboxamide;
5-((2-((4-((3-Chloro-4-(pyridin-3-yl)benzyl)amino)butyl)amino)ethyl)amino)benzo[c][2,6]naphthyridine-8-carboxamide;
5-((2-((4-((3-Chloro-4-(pyridin-4-yl)benzyl)amino)butyl)amino)ethyl)amino)benzo[c][2,6]naphthyridine-8-carboxamide;
5-((2-((4-(((1H-Indol-2-yl)methyl)amino)butyl)amino)ethyl)amino)benzo[c][2,6]naphthyridine-8-carboxamide;
5-((2-((4-(((5-Chloro-1H-indol-2-yl)methyl)amino)butyl)amino)ethyl)amino)benzo[c][2,6]naphthyridine-8-carboxamide;
5-((2-((4-(((4-Methyl-3-phenyl-1H-pyrazol-5-yl)methyl)amino)butyl)amino)ethyl)amino)benzo[c][2,6]naphthyridine-8-carboxamide;
5-((2-((4-(((4-chloro-3-phenyl-1H-pyrazol-5-yl)methyl)amino)butyl)amino)ethyl)amino)benzo[c][2,6]naphthyridine-8-carboxamide;
5-((2-((4-(((3-Phenyl-1H-pyrazol-5-yl)methyl)amino)butyl)amino)ethyl)amino)benzo[c][2,6]naphthyridine-8-carboxamide;
5-((2-((4-(((5-Fluoro-1H-indol-2-yl)methyl)amino)butyl)amino)ethyl)amino)benzo[c][2,6]naphthyridine-8-carboxamide;
5-((2-((4-((3-Chloro-4-fluorobenzyl)amino)butyl)amino)ethyl)amino)benzo[c][2,6]naphthyridine-8-carboxamide;
5-((2-((4-((3-Chloro-4-(trifluoromethoxy)benzyl)amino)butyl)amino)ethyl)amino)benzo[c][2,6]naphthyridine-8-carboxamide;
5-((2-((4-(((6-Chloro-1H-indol-2-yl)methyl)amino)butyl)amino)ethyl)amino)benzo[c][2,6]naphthyridine-8-carboxamide;
5-((2-((4-(((6-Fluoro-1H-indol-2-yl)methyl)amino)butyl)amino)ethyl)amino)benzo[c][2,6]naphthyridine-8-carboxamide;
5-((2-((4-(((5-Methoxy-1H-indol-2-yl)methyl)amino)butyl)amino)ethyl)amino)benzo[c][2,6]naphthyridine-8-carboxamide;
5-((2-((4-(((4-methyl-5-phenylpyridin-2-yl)methyl)amino)butyl)amino)ethyl)amino)benzo[c][2,6]naphthyridine-8-carboxamide;
5-((2-((4-(((5-(Trifluoromethyl)-1H-indol-2-yl)methyl)amino)butyl)amino)ethyl)amino)benzo[c][2,6]naphthyridine-8-carboxamide;
5-((2-((4-(((2-Methoxy-[1,1′-biphenyl]-4-yl)methyl)amino)butyl)amino)ethyl)amino)benzo[c][2,6]naphthyridine-8-carboxamide;
5-((2-((4-(((2-Cyano-[1,1′-biphenyl]-4-yl)methyl)amino)butyl)amino)ethyl)amino)benzo[c][2,6]naphthyridine-8-carboxamide;
5-((2-((4-(((2-Fluoro-[1,1′-biphenyl]-4-yl)methyl)amino)butyl)amino)ethyl)amino)benzo[c][2,6]naphthyridine-8-carboxamide;
5-((2-((4-(((2-Chloro-2′-(cyanomethyl)-[1,1′-biphenyl]-4-yl)methyl)amino)butyl)amino)ethyl)amino)benzo[c][2,6]naphthyridine-8-carboxamide;
5-((2-((4-((3-Chloro-4-(pyrazin-2-yl)benzyl)amino)butyl)amino)ethyl)amino)benzo[c][2,6]naphthyridine-8-carboxamide;
5-((2-((4-(((5-Chloro-6-phenylpyridin-3-yl)methyl)amino)butyl)amino)ethyl)amino)benzo[c][2,6]naphthyridine-8-carboxamide;
5-((2-((4-(((2-Chloro-2′-(2-hydroxyethyl)-[1,1′-biphenyl]-4-yl)methyl)amino)butyl)amino)ethyl)amino)benzo[c][2,6]naphthyridine-8-carboxamide;
5-((2-((4-(((6-Chloroquinolin-2-yl)methyl)amino)butyl)amino)ethyl)amino)benzo[c][2,6]naphthyridine-8-carboxamide;
5-((2-((4-(((6-(Trifluoromethyl) quinolin-2-yl)methyl)amino)butyl)amino)ethyl)amino)benzo[c][2,6]naphthyridine-8-carboxamide;
5-((2-((4-(((5-Phenylthiazol-2-yl)methyl)amino)butyl)amino)ethyl)amino)benzo[c][2,6]naphthyridine-8-carboxamide;
5-((2-((4-((3-Chloro-4-cyclobutoxybenzyl)amino)butyl)amino)ethyl)amino)benzo[c][2,6]naphthyridine-8-carboxamide;
5-((2-((4-((3-Cyano-4-cyclobutoxybenzyl)amino)butyl)amino)ethyl)amino)benzo[c][2,6]naphthyridine-8-carboxamide;
5-((2-((4-((3-Cyano-4-(trifluoromethoxy)benzyl)amino)butyl)amino)ethyl)amino)benzo[c][2,6]naphthyridine-8-carboxamide;
5-((3-(3-(((2-Chloro-[1,1′-biphenyl]-4-yl)methyl)amino)propanamido)propyl)amino)benzo[c][2,6]naphthyridine-8-carboxylic acid;
5-((2-((3-(((2-Chloro-[1,1′-biphenyl]-4-yl)methyl)amino)propyl)amino)ethyl)amino)benzo[c][2,6]naphthyridine-8-carboxylic acid;
6-(3-(3-(((2-Chloro-[1,1′-biphenyl]-4-yl)methyl)amino)propanamido)propyl)-5,6-dihydroimidazo[1,5-c]quinazoline-8-carboxylic acid;
5-((2-((4-(((2-chloro-[1,1′-biphenyl]-4-yl)methyl)amino)butyl)amino)ethyl)amino)benzo[c][2,6]naphthyridine-8-carbonitrile;
N-(5-((2-((4-(((2-chloro-[1,1′-biphenyl]-4-yl)methyl)amino)butyl)amino)ethyl)amino)benzo[c][2,6]naphthyridin-8-yl)acetamide;
5-((2-(4-(((2-Chloro-[1,1′-biphenyl]-4-yl)methyl)amino)butanamido)ethyl)amino)benzo[c][2,6]naphthyridine-8-carboxamide;
5-((3-(3-(((2-Chloro-[1,1′-biphenyl]-4-yl)methyl)amino)propoxy)propyl)amino)benzo[c][2,6]naphthyridine-8-carboxamide;
5-((2-(4-(((2-Chloro-[1,1′-biphenyl]-4-yl)methyl)amino)-N-methylbutanamido)ethyl)(methyl)amino)benzo[c][2,6]naphthyridine-8-carboxamide;
5-((3-(3-(((2-Chloro-[1,1′-biphenyl]-4-yl)methyl)amino)propanamido)propyl)amino)benzo[c][2,6]naphthyridine-8-carboxamide;
5-((3-(3-(((2-chloro-[1,1′-biphenyl]-4-yl)methyl)amino)-N-methylpropanamido)propyl)amino)benzo[c][2,6]naphthyridine-8-carboxamide;
5-((2-(4-(((2-Chloro-[1,1′-biphenyl]-4-yl)methyl)amino)butanamido)ethyl)(methyl)amino)benzo[c][2,6]naphthyridine-8-carboxamide;
5-((3-(3-(((2-Chloro-[1,1′-biphenyl]-4-yl)methyl)amino)propanamido)-3-methylbutyl)amino)benzo[c][2,6]naphthyridine-8-carboxamide;
5-((2-(4-(((2-Chloro-[1,1′-biphenyl]-4-yl)methyl)amino)-N-methylbutanamido)ethyl)amino)benzo[c][2,6]naphthyridine-8-carboxamide;
5-((2-(4-(2-(((2-Chloro-[1,1′-biphenyl]-4-yl)methyl)amino)ethyl)-1H-1,2,3-triazol-1-yl)ethyl)amino)benzo[c][2,6]naphthyridine-8-carboxamide;
5-((2-(5-(2-(((2-Chloro-[1,1′-biphenyl]-4-yl)methyl)amino)ethyl)-1,2,4-oxadiazol-3-yl)ethyl)amino)benzo[c][2,6]naphthyridine-8-carboxamide;
5-((3-(N-(2-(((2-chloro-[1,1′-biphenyl]-4-yl)methyl)amino)ethyl)sulfamoyl)propyl)amino)benzo[c][2,6]naphthyridine-8-carboxamide;
5-((2-(2-(2-(((2-chloro-[1,1′-biphenyl]-4-yl)methyl)amino)ethyl)oxazol-5-yl)ethyl)amino)benzo[c][2,6]naphthyridine-8-carboxamide;
5-((2-(3-(2-(((2-Chloro-[1,1′-biphenyl]-4-yl)methyl)amino)ethyl)-1,2,4-oxadiazol-5-yl)ethyl)amino)benzo[c][2,6]naphthyridine-8-carboxamide;
5-((2-(5-(2-(((2-Chloro-[1,1′-biphenyl]-4-yl)methyl)amino)ethyl)-4H-1,2,4-triazol-3-yl)ethyl)amino)benzo[c][2,6]naphthyridine-8-carboxamide;
5-((2-(4-(2-(((2-Chloro-[1,1′-biphenyl]-4-yl)methyl)amino)ethyl)-1H-imidazol-1-yl)ethyl)amino)benzo[c][2,6]naphthyridine-8-carboxamide;
5-((2-(2-(2-((3-Chloro-4-(trifluoromethoxy)benzyl)amino)ethyl)oxazol-5-yl)ethyl)amino)benzo[c][2,6]naphthyridine-8-carboxamide;
5-((2-(5-(2-((3-Chloro-4-(trifluoromethoxy)benzyl)amino)ethyl)-4H-1,2,4-triazol-3-yl)ethyl)amino)benzo[c][2,6]naphthyridine-8-carboxamide;
5-((2-(2-(2-(((2-Chloro-[1,1′-biphenyl]-4-yl)methyl)amino)ethyl)-1H-imidazol-5-yl)ethyl)amino)benzo[c][2,6]naphthyridine-8-carboxamide;
5-((2-(4-(2-((3-Chloro-4-(trifluoromethoxy)benzyl)amino)ethyl)-1H-imidazol-1-yl)ethyl)amino)benzo[c][2,6]naphthyridine-8-carboxamide;
5-((2-((4-((3-Chloro-4-(trifluoromethoxy)benzyl)amino)butyl)(methyl)amino)ethyl)amino)benzo[c][2,6]naphthyridine-8-carboxamide;
3-((3-Chloro-4-(trifluoromethoxy)benzyl)amino)-N-(3-((8-cyanobenzo[c][2,6]naphthyridin-5-yl)amino)propyl)propenamide;
5-((2-(4-(2-(((2-Chloro-[1,1′-biphenyl]-4-yl)methyl)amino)ethyl)-1H-1,2,3-triazol-1-yl)ethyl)amino)benzo[c][2,6]naphthyridine-8-carboxylic acid;
5-((3-(4-((((2-Chloro-[1,1′-biphenyl]-4-yl)methyl)amino)methyl)-1H-1,2,3-triazol-1-yl)propyl)amino)benzo[c][2,6]naphthyridine-8-carboxamide;
4-((3-(3-(((2-Chloro-[1,1′-biphenyl]-4-yl)methyl)amino)propanamido)propyl)amino)-3H-pyrazolo[3,4-c]quinoline-7-carboxylic acid;
5-((2-((4-((3-Chloro-4-(trifluoromethoxy)benzyl)amino)butyl)amino)ethyl)amino)benzo[c][2,6]naphthyridine-8-carboxylic acid;
6-(3-(3-(((2-Chloro-[1,1′-biphenyl]-4-yl)methyl)amino)propanamido)propyl)-5-oxo-5,6-dihydrobenzo[c][2,6]naphthyridine-8-carboxylic acid;
5-((2-((4-(((5-(Trifluoromethyl)-1H-indol-2-yl)methyl)amino)butyl)amino)ethyl)amino)benzo[c][2,6]naphthyridine-8-carboxylic acid;
5-((2-(((3-(((2-Chloro-[1,1′-biphenyl]-4-yl)methyl)amino)cyclobutyl)methyl)amino)ethyl)amino)benzo[c][2,6]naphthyridine-8-carboxamide;
5-((2-(3-(2-(((2-Chloro-[1,1′-biphenyl]-4-yl)methyl)amino)ethyl)ureido)ethyl)amino)benzo[c][2,6]naphthyridine-8-carboxamide;
5-((2-(3-(((2-Chloro-[1,1′-biphenyl]-4-yl)methyl)amino)propylsulfonamido)ethyl)amino)benzo[c][2,6]naphthyridine-8-carboxamide;
5-((3-(2-(((2-Chloro-[1,1′-biphenyl]-4-yl)methyl)amino)ethylsulfonamido)propyl)amino)benzo[c][2,6]naphthyridine-8-carboxamide;
N¹-(2-((8-(2H-tetrazol-5-yl)benzo[c][2,6]naphthyridin-5-yl)amino)ethyl)-N⁴-((2-chloro-[1,1′-biphenyl]-4-yl)methyl) butane-1,4-diamine;
5-((2-((4-((3-Chloro-4-(pyrimidin-5-yl)benzyl)amino)butyl)amino)ethyl)amino)benzo[c][2,6]naphthyridine-8-carboxamide;
5-((2-((4-(((6-Methyl-5-phenylpyridin-2-yl)methyl)amino)butyl)amino)ethyl)amino)benzo[c][2,6]naphthyridine-8-carboxamide;
5-((2-((4-((3-Chloro-4-(pyridin-2-yl)benzyl)amino)butyl)amino)ethyl)amino)benzo[c][2,6]naphthyridine-8-carboxamide;
5-((2-((4-(((2-Chloro-2′-(2-hydroxyethoxy)-[1,1′-biphenyl]-4-yl)methyl)amino)butyl)amino)ethyl)amino)benzo[c][2,6]naphthyridine-8-carboxamide;
5-(3-(3-((3-chloro-4-(trifluoromethoxy)benzyl)amino)propanamido)propyl)-4-oxo-4,5-dihydro-3H-pyrazolo[3,4-c]quinoline-7-carboxylic acid;
5-((4-(2-(((2-chloro-[1,1′-biphenyl]-4-yl)methyl)amino)ethoxy)butyl)amino)benzo[c][2,6]naphthyridine-8-carboxamide;
5-((2-(N-(3-(((2-chloro-[1,1′-biphenyl]-4-yl)methyl)amino)propyl)sulfamoyl)ethyl)amino)benzo[c][2,6]naphthyridine-8-carboxamide;
5-((2-(1-(2-(((2-Chloro-[1,1′-biphenyl]-4-yl)methyl)amino)ethyl)-1H-pyrazol-4-yl)ethyl)amino)benzo[c][2,6]naphthyridine-8-carboxamide;
5-((2-(1-(2-((3-Chloro-4-(trifluoromethoxy)benzyl)amino)ethyl)-1H-pyrazol-4-yl)ethyl)amino)benzo[c][2,6]naphthyridine-8-carboxamide;
5-((3-(3-((3-Chloro-4-(trifluoromethoxy)benzyl)amino)propoxy)propyl)amino)benzo[c][2,6]naphthyridine-8-carboxamide;
5-((4-(2-(((2-Chloro-[1,1′-biphenyl]-4-yl)methyl)amino)ethyl)phenethyl)amino)benzo[c][2,6]naphthyridine-8-carboxamide;
5-((4-(2-((3-Chloro-4-(trifluoromethoxy)benzyl)amino)ethoxy)butyl)amino)benzo[c][2,6]naphthyridine-8-carboxamide;
5-((3-(2-(((2-Chloro-[1,1′-biphenyl]-4-yl)methyl)amino)ethyl)phenethyl)amino)benzo[c][2,6]naphthyridine-8-carboxamide;
5-((3-((3-((3-Chloro-4-(trifluoromethoxy)benzyl)amino)propyl)amino)-3-oxopropyl)amino)benzo[c][2,6]naphthyridine-8-carboxamide;
5-((2-(4-((3-Chloro-4-(trifluoromethoxy)benzyl)amino)butanamido)ethyl)amino)benzo[c][2,6]naphthyridine-8-carboxamide;
5-((2-(4-((((2-Chloro-[1,1′-biphenyl]-4-yl)methyl)amino)methyl)piperidin-1-yl)ethyl)amino)benzo[c][2,6]naphthyridine-8-carboxamide;
5-((2-(5-(((2-Chloro-[1,1′-biphenyl]-4-yl)methyl)amino)-2H-indazol-2-yl)ethyl)amino)benzo[c][2,6]naphthyridine-8-carboxamide;
5-((2-(6-(((2-Chloro-[1,1′-biphenyl]-4-yl)methyl)amino)-1H-benzo[d]imidazol-2-yl)ethyl)amino)benzo[c][2,6]naphthyridine-8-carboxamide;
5-((2-((3-(((3-Chloro-4-(trifluoromethoxy)benzyl)amino)methyl)cyclobutyl)amino)ethyl)amino)benzo[c][2,6]naphthyrid ine-8-carboxamide;
5-((2-(3-(2-((3-Chloro-4-(trifluoromethoxy)benzyl)amino)ethyl) pyrrolidin-1-yl)ethyl)amino)benzo[c][2,6]naphthyridine-8-carboxamide;
5-((2-(3-(N-(3-chlorobenzyl)sulfamoyl)propoxy)ethyl)amino)benzo[c][2,6]naphthyridine-8-carboxamide;
(S)-5-((2-((4-((3-Chlorobenzyl)amino)pentyl)oxy)ethyl)amino)benzo[c][2,6]naphthyridine-8-carboxamide;
5-((4-(4-(((2-Chloro-[1,1′-biphenyl]-4-yl)methyl)amino)-1H-1,2,3-triazol-1-yl)butyl)amino)benzo[c][2,6]naphthyridine-8-carboxamide;
5-((3-(3-((3-Chlorobenzyl)amino)propanamido)propyl)amino)benzo[c][2,6]naphthyridine-8-carboxamide;
5-((3-(3-((3-Chloro-4-(trifluoromethoxy)benzyl)amino)propanamido)propyl)amino)benzo[c][2,6]naphthyridine-8-carboxamide;
5-((3-(3-((3-Chloro-4-cyclopropoxybenzyl)amino)propanamido)propyl)amino)benzo[c][2,6]naphthyridine-8-carboxamide;
5-((3-(3-((3-Chloro-4-cyclopropylbenzyl)amino)propanamido)propyl)amino)benzo[c][2,6]naphthyridine-8-carboxamide;
5-((3-(3-((3-chloro-4-(cyclopentyloxy)benzyl)amino)propanamido)propyl)amino)benzo[c][2,6]naphthyridine-8-carboxide;
5-((3-(3-((3-chloro-4-cyclobutoxybenzyl)amino)propanamido)propyl)amino)benzo[c][2,6]naphthyridine-8-carboxamide;
5-((3-(3-(((6-Chloro-1H-indol-2-yl)methyl)amino)propanamido)propyl)amino)benzo[c][2,6]naphthyridine-8-carboxamide;
5-((3-(3-((3-Fluoro-4-(trifluoromethoxy)benzyl)amino)propanamido)propyl)amino)benzo[c][2,6]naphthyridine-8-carboxamide;
5-((3-(3-((3,4-Dichlorobenzyl)amino)propanamido)propyl)amino)benzo[c][2,6]naphthyridine-8-carboxamide;
5-((3-(3-((3,5-Dichlorobenzyl)amino)propanamido)propyl)amino)benzo[c][2,6]naphthyridine-8-carboxamide;
5-((3-(3-((3-Chloro-4-(trifluoromethyl)benzyl)amino)propanamido)propyl)amino)benzo[c][2,6]naphthyridine-8-carboxamide;
5-((3-(3-((3-Chloro-5-fluorobenzyl)amino)propanamido)propyl)amino)benzo[c][2,6]naphthyridine-8-carboxamide;
5-((3-(3-(((6-Chloro-1-methyl-1H-indol-2-yl)methyl)amino)propanamido)propyl)amino)benzo[c][2,6]naphthyridine-8-carboxamide;
5-((3-(3-(((4-Chloro-3-phenyl-1H-pyrazol-5-yl)methyl)amino)propanamido)propyl)amino)benzo[c][2,6]naphthyridine-8-carboxamide;
5-((3-(3-(((4-Chloro-1-methyl-3-phenyl-1H-pyrazol-5-yl)methyl)amino)propanamido)propyl)amino)benzo[c][2,6]naphthyridine-8-carboxamide;
5-((3-(3-(((6-(Trifluoromethyl)pyridin-3-yl)methyl)amino)propanamido)propyl)amino)benzo[c][2,6]naphthyridine-8-carboxamide;
5-((3-(3-(((5-Chloropyridin-3-yl)methyl)amino)propanamido)propyl)amino)benzo[c][2,6]naphthyridine-8-carboxamide;
5-((3-(3-(((5-(Trifluoromethyl)pyridin-2-yl)methyl)amino)propanamido)propyl)amino)benzo[c][2,6]naphthyridine-8-carboxamide;
5-((3-(3-((3-Chloro-4-cyclopentylbenzyl)amino)propanamido)propyl)amino)benzo[c][2,6]naphthyridine-8-carboxamide;
5-((3-(3-((3-(2-Hydroxyethyl)benzyl)amino)propanamido)propyl)amino)benzo[c][2,6]naphthyridine-8-carboxamide;
5-((3-(3-((3-(Hydroxymethyl)benzyl)amino)propanamido)propyl)amino)benzo[c][2,6]naphthyridine-8-carboxamide;
5-((3-(3-((3-(2-Hydroxyethoxy)benzyl)amino)propanamido)propyl)amino)benzo[c][2,6]naphthyridine-8-carboxamide;
5-((3-(3-((3-Cyanobenzyl)amino)propanamido)propyl)amino)benzo[c][2,6]naphthyridine-8-carboxamide;
5-((3-(3-((3-Chloro-4-cyclopentylbenzyl)amino)propanamido)propyl)amino)benzo[c][2,6]naphthyridine-8-carboxamide;
5-((3-(3-(((6-Methylpyridin-2-yl)methyl)amino)propanamido)propyl)amino)benzo[c][2,6]naphthyridine-8-carboxamide;
5-((3-(3-((3-Cyano-4-(trifluoromethoxy)benzyl)amino)propanamido)propyl)amino)benzo[c][2,6]naphthyridine-8-carboxamide;
5-((3-(3-((3-Chloro-5-(hydroxymethyl)benzyl)amino)propanamido)propyl)amino)benzo[c][2,6]naphthyridine-8-carboxamide;
5-((3-(3-((3-(Cyanomethyl)benzyl)amino)propanamido)propyl)amino)benzo[c][2,6]naphthyridine-8-carboxamide;
5-((3-(3-((3-cyano-4-cyclobutoxybenzyl)amino)propanamido)propyl)amino)benzo[c][2,6]naphthyridine-8-carboxamide;
5-((3-(3-(((2-cyano-[1,1′-biphenyl]-4-yl)methyl)amino)propanamido)propyl)amino)benzo[c][2,6]naphthyridine-8-carboxamide;
5-((3-(3-(((6-(trifluoromethoxy) quinolin-2-yl)methyl)amino)propanamido)propyl)amino)benzo[c][2,6]naphthyridine-8-carboxamide;
5-((3-(3-((3-cyano-4-cyclopropylbenzyl)amino)propanamido)propyl)amino)benzo[c][2,6]naphthyridine-8-carboxamide;
5-((3-(3-(((6-Cyclopropylpyridin-3-yl)methyl)amino)propanamido)propyl)amino)benzo[c][2,6]naphthyridine-8-carboxamide;
5-((3-(3-(((5-Cyclopropylpyridin-2-yl)methyl)amino)propanamido)propyl)amino)benzo[c][2,6]naphthyridine-8-carboxamide;
5-((3-(3-((3,5-Difluoro-4-(trifluoromethoxy)benzyl)amino)propanamido)propyl)amino)benzo[c][2,6]naphthyridine-8-carboxamide;
5-((3-(3-((3-Chloro-5-(cyanomethyl)benzyl)amino)propanamido)propyl)amino)benzo[c][2,6]naphthyridine-8-carboxamide;
5-((2-(2-(2-((3-Chloro-4-(trifluoromethoxy)benzyl)amino)ethoxy) ethoxy)ethyl)amino)benzo[c][2,6]naphthyridine-8-carboxamide;
5-((2-(4-(2-((3-Chloro-4-(trifluoromethoxy)benzyl)amino)ethyl)-1H-1,2,3-triazol-1-yl)ethyl)amino)benzo[c][2,6]naphthyridine-8-carboxamide;
5-((2-(4-(2-(((6-Methyl-5-phenylpyridin-2-yl)methyl)amino)ethyl)-1H-1,2,3-triazol-1-yl)ethyl)amino)benzo[c][2,6]naphthyridine-8-carboxamide;
5-((2-(4-(2-((3-chlorobenzyl)amino)ethyl)-1H-1,2,3-triazol-1-yl)ethyl)amino)benzo[c][2,6]naphthyridine-8-carboxamide;
5-((2-(4-(2-(((5-(Trifluoromethyl)-1H-indol-2-yl)methyl)amino)ethyl)-1H-1,2,3-triazol-1-yl)ethyl)amino)benzo[c][2,6]naphthyridine-8-carboxamide;
5-((2-(4-(2-((3-chloro-5-fluorobenzyl)amino)ethyl)-1H-1,2,3-triazol-1-yl)ethyl)amino)benzo[c][2,6]naphthyridine-8-carboxamide;
5-((2-(4-(2-((3,5-dichlorobenzyl)amino)ethyl)-1H-1,2,3-triazol-1-yl)ethyl)amino)benzo[c][2,6]naphthyridine-8-carboxamide;
5-((2-(4-(2-(((6-Chloro-1H-indol-2-yl)methyl)amino)ethyl)-1H-1,2,3-triazol-1-yl)ethyl)amino)benzo[c][2,6]naphthyridine-8-carboxamide;
5-((2-(4-(2-((3-Chloro-4-cyclobutoxybenzyl)amino)ethyl)-1H-1,2,3-triazol-1-yl)ethyl)amino)benzo[c][2,6]naphthyridine-8-carboxamide;
5-((2-(4-(2-((3-Chloro-4-cyclopropylbenzyl)amino)ethyl)-1H-1,2,3-triazol-1-yl)ethyl)amino)benzo[c][2,6]naphthyridine-8-carboxamide;
5-((2-(4-(2-((3-fluoro-4-(trifluoromethoxy)benzyl)amino)ethyl)-1H-1,2,3-triazol-1-yl)ethyl)amino)benzo[c][2,6]naphthyridine-8-carboxamide;
5-((2-(4-(2-(((2-Chloro-2′-(2-hydroxyethoxy)-[1,1′-biphenyl]-4-yl)methyl)amino)ethyl)-1H-1,2,3-triazol-1-yl)ethyl)amino)benzo[c][2,6]naphthyridine-8-carboxamide;
5-((2-(4-(2-(((5-Chloro-6-phenylpyridin-3-yl)methyl)amino)ethyl)-1H-1,2,3-triazol-1-yl)ethyl)amino)benzo[c][2,6]naphthyridine-8-carboxamide;
5-((2-(4-(2-(((1-Methyl-5-(trifluoromethyl)-1H-indol-2-yl)methyl)amino)ethyl)-1H-1,2,3-triazol-1-yl)ethyl)amino)benzo[c][2,6]naphthyridine-8-carboxamide;
5-((3-(3-((3-Chloro-4-cyclobutoxybenzyl)amino)propanamido)propyl)amino)benzo[c][2,6]naphthyridine-8-carboxylic acid;
5-((3-(3-((3-Chloro-4-cyclobutoxybenzyl)amino)-N-methylpropanamido)propyl)amino)benzo[c][2,6]naphthyridine-8-carboxamide;
5-((3-(3-((3-Chloro-4-(trifluoromethoxy)benzyl)amino)propanamido)propyl)amino)benzo[c][2,6]naphthyridine-8-carboxylic acid;
5-((3-(3-((3-Chloro-4-(trifluoromethoxy)benzyl)amino)propoxy)propyl)amino)benzo[c][2,6]naphthyridine-8-carboxylic acid;
5-((3-((3-((3-Chloro-4-(trifluoromethoxy)benzyl)amino)propyl)amino)-3-oxopropyl)amino)benzo[c][2,6]naphthyridine-8-carboxylic acid;
5-((4-(2-((3-Chloro-4-(trifluoromethoxy)benzyl)amino)ethoxy)butyl)amino)benzo[c][2,6]naphthyridine-8-carboxylic acid;
5-((4-((2-((3-Chloro-4-(trifluoromethoxy)benzyl)amino)ethyl)amino)-4-oxobutyl)amino)benzo[c][2,6]naphthyridine-8-carboxylic acid;
5-((2-(4-((3-Chloro-4-(trifluoromethoxy)benzyl)amino)butanamido)ethyl)amino)benzo[c][2,6]naphthyridine-8-carboxylic acid;
5-(2-(4-((3-Chloro-4-(trifluoromethoxy)benzyl)amino)butoxy)ethyl)-4-oxo-4,5-dihydro-3H-pyrazolo[3,4-c]quinoline-7-carboxylic acid;
5-(4-(3-((3-Chloro-4-(trifluoromethoxy)benzyl)amino)propoxy)piperidin-1-yl)benzo[c][2,6]naphthyridine-8-carboxylic acid;
5-(4-(4-((3-Chloro-4-(trifluoromethoxy)benzyl)amino)butyl)piperazin-1-yl)benzo[c][2,6]naphthyridine-8-carboxylic acid;
(R)-5-(3-(3-((3-Chloro-4-(trifluoromethoxy)benzyl)amino)propoxy)pyrrolidin-1-yl)benzo[c][2,6]naphthyridine-8-carboxylic acid;
(S)-5-(3-(3-((3-Chloro-4-(trifluoromethoxy)benzyl)amino)propoxy)pyrrolidin-1-yl)benzo[c][2,6]naphthyridine-8-carboxylic acid;
N-(3-((8-(2H-tetrazol-5-yl)benzo[c][2,6]naphthyridin-5-yl)amino)propyl)-3-((3-chloro-4-cyclobutoxybenzyl)amino)propenamide;
N-(3-((8-(2H-tetrazol-5-yl)benzo[c][2,6]naphthyridin-5-yl)amino)propyl)-3-((3-chloro-4-(trifluoromethoxy)benzyl)amino)propenamide;
N-(2-(4-(2-(((2-chloro-[1,1′-biphenyl]-4-yl)methyl)amino)ethyl)-1H-1,2,3-triazol-1-yl)ethyl)-8-(2H-tetrazol-5-yl)benzo[c][2,6]naphthyridin-5-amine;
5-(3-(3-((3-Chloro-4-(trifluoromethoxy)benzyl)amino)propanamido)propyl)-4-oxo-4,5-dihydro-3H-pyrazolo[3,4-c]quinoline-7-carboxamide;
5-((2-((3-(((2-Chloro-[1,1′-biphenyl]-4-yl)methyl)amino)cyclobutyl methoxy)ethyl)amino)benzo[c][2,6]naphthyridine-8-carboxamide;
5-((2-((5-((3-Chlorophenyl)amino)pentyl)oxy)ethyl)amino)benzo[c][2,6]naphthyridine-8-carboxamide;
5-(3-(4-(((5-Cyclobutoxy-1H-indol-2-yl)methyl)amino)butoxy)azetidin-1-yl)benzo[c][2,6]naphthyridine-8-carboxamide;
(S)-5-(3-(3-((3-chloro-4-(trifluoromethoxy)benzyl)amino)propoxy)pyrrolidin-1-yl)benzo[c][2,6]naphthyridine-8-carboxamide;
5-(4-(4-((3,5-Difluoro-4-(trifluoromethoxy)benzyl)amino)-1H-1,2,3-triazol-1-yl)butoxy)benzo[c][2,6]naphthyridine-8-carboxylic acid;
5-(2-(4-(2-((3,5-Difluoro-4-(trifluoromethoxy)benzyl)amino)ethyl)-1H-1,2,3-triazol-1-yl)ethoxy)benzo[c][2,6]naphthyridine-8-carboxylic acid;
5-(2-((3-((3,5-Difluoro-4-(trifluoromethoxy)benzyl)amino)cyclobutyl)methoxy)ethoxy)benzo[c][2,6]naphthyridine-8-carboxylic acid; or
(S)-5-(2-((4-((3,5-difluoro-4-(trifluoromethoxy)benzyl)amino)pentyl)oxy)ethoxy)benzo[c][2,6]naphthyridine-8-carboxylic acid.

Though the present invention may relate to any compound or particular group of compounds defined herein by way of optional, preferred or suitable features or otherwise in terms of particular embodiments, the present invention may also relate to any compound or particular group of compounds that specifically excludes said optional, preferred or suitable features or particular embodiments.

Suitably, the present invention excludes any individual compounds not possessing the biological activity defined herein.

### Salts and Solvates

The compounds (including final products and intermediates) described herein may be isolated and used per se or may be isolated in the form of a salt, suitably pharmaceutically acceptable salts. It should be understood that the terms “salt(s)” and “salt form(s)” used by themselves or in conjunction with another term or terms encompasses all inorganic and organic salts, including industrially acceptable salts, as defined herein, and pharmaceutically acceptable salts, as defined herein, unless otherwise specified. As used herein, industrially acceptable salts are salts that are generally suitable for manufacturing and/or processing (including purification) as well as for shipping and storage, but may not be salts that are typically administered for clinical or therapeutic use. Industrially acceptable salts may be prepared on a laboratory scale, i.e. multi-gram or smaller, or on a larger scale, i.e. up to and including a kilogram or more.

Pharmaceutically acceptable salts, as used herein, are salts that are generally chemically and/or physically compatible with the other ingredients comprising a formulation, and/or are generally physiologically compatible with the recipient thereof. Pharmaceutically acceptable salts may be prepared on a laboratory scale, i.e. multi-gram or smaller, or on a larger scale, i.e. up to and including a kilogram or more. It should be understood that pharmaceutically acceptable salts are not limited to salts that are typically administered or approved by the FDA or equivalent foreign regulatory body for clinical or therapeutic use in humans. A practitioner of ordinary skill will readily appreciate that some salts are both industrially acceptable as well as pharmaceutically acceptable salts. It should be understood that all such salts, including mixed salt forms, are within the scope of the application.

In one embodiment, the compounds of Formula I and sub-formulae thereof are isolated as pharmaceutically acceptable salts.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulfuric, phosphoric, trifluoroacetic, formic, citric or maleic acid. In addition a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

In general, salts of the present application can be prepared in situ during the isolation and/or purification of a compound (including intermediates), or by separately reacting the compound (or intermediate) with a suitable organic or inorganic acid or base (as appropriate) and isolating the salt thus formed. The degree of ionisation in the salt may vary from completely ionised to almost non-ionised. In practice, the various salts may be precipitated (with or without the addition of one or more co-solvents and/or anti-solvents) and collected by filtration or the salts may be recovered by evaporation of solvent(s). Salts of the present application may also be formed via a “salt switch” or ion exchange/double displacement reaction, i.e. reaction in which one ion is replaced (wholly or in part) with another ion having the same charge. One skilled in the art will appreciate that the salts may be prepared and/or isolated using a single method or a combination of methods.

Representative salts include, but are not limited to, acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate, trifluoroacetate and the like. Other examples of representative salts include alkali or alkaline earth metal cations such as, but not limited to, sodium, lithium, potassium, calcium, magnesium, and the like, as well as non-toxic ammonium, quaternary ammonium and amine cations including, but not limited to, ammonium, tetramethylammonium, tetraethylammonium, lysine, arginine, benzathine, choline, tromethamine, diolamine, glycine, meglumine, olamine and the like.

Certain compounds of the Formula I and sub-formulae thereof may exist in solvated as well as unsolvated forms such as, for example, hydrated forms. It is to be understood that the invention encompasses all such solvated forms that possess the biological activity described herein.

### Polymorphs

It is also to be understood that certain compounds of the Formula I and sub-formulae thereof may exhibit polymorphism, and that the invention encompasses all such forms that possess the biological activity described herein.

### N-Oxides

Compounds of the Formula I and sub-formulae thereof containing an amine function may also form N-oxides. A reference herein to a compound of the Formula I and sub-formulae thereof that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-Oxides can be formed by treatment of the corresponding amine with an oxidizing agent such as, but not limited to, hydrogen peroxide or a per-acid (e.g. a peroxycarboxylic acid), see for example Advanced Organic Chemistry, by Jerry March 4th Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (mCPBA), for example, in an inert solvent such as, but not limited to, dichloromethane.

### Tautomers

Compounds of the Formula I and sub-formulae thereof may exist in a number of different tautomeric forms and references to compounds of the Formula I and sub-formulae thereof include all such forms. For the avoidance of doubt, where a compound can exist in one of several tautomeric forms, and only one is specifically described or shown, all others are nevertheless embraced by Formula I and sub-formulae thereof. Examples of tautomeric forms include keto-, enol-, and enolate-forms, as in, for example, the following tautomeric pairs: keto/enol (illustrated below), pyrimidone/hydroxypyrimidine, imine/enamine, amide/imino alcohol, amidine/amidine, nitroso/oxime, thioketone/enethiol, and nitro/aci-nitro.

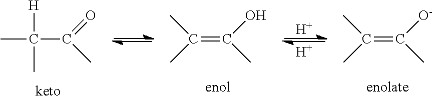

### Isomers

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

Certain compounds of Formula I and sub-formulae thereof may have one or more asymmetric centres and therefore can exist in a number of stereoisomeric configurations. Consequently, such compounds can be synthesized and/or isolated as mixtures of enantiomers and/or as individual (pure) enantiomers, and, in the case of two or more asymmetric centres, single diastereomers and/or mixtures of diastereomers. It should be understood that the present application includes all such enantiomers and diastereomers and mixtures thereof in all ratios.

### Isotopes

The compounds of the present invention are described herein using structural formulas that do not specifically recite the mass numbers or the isotope ratios of the constituent atoms. As such it is intended that the present application includes compounds in which the constituent atoms are present in any ratio of isotope forms. For example, carbon atoms may be present in any ratio of ¹²C, ¹³C, and ¹⁴C; hydrogen atoms may be present in any ratio of ¹H, ²H, and ³H; etc. Preferably, the constituent atoms in the compounds of the present invention are present in their naturally occurring ratios of isotope forms.

### Prodrugs and Metabolites

The compounds of Formula I and sub-formulae thereof may be administered in the form of a pro-drug which is broken down in the human or animal body to release a compound of the invention. A pro-drug may be used to alter the physical properties and/or the pharmacokinetic properties of a compound of the invention. A pro-drug can be formed when the compound of the invention contains a suitable group or substituent to which a property-modifying group can be attached. Examples of pro-drugs include in vivo cleavable ester derivatives that may be formed at a carboxy group or a hydroxy group in a compound of the Formula I and in-vivo cleavable amide derivatives that may be formed at a carboxy group or an amino group in a compound of the Formula I and sub-formulae thereof.

Accordingly, the present invention includes those compounds of the Formula I and sub-formulae thereof as defined hereinbefore when made available by organic synthesis and when made available within the human or animal body by way of cleavage of a pro-drug thereof. Accordingly, the present invention includes those compounds of the Formula I that are produced by organic synthetic means and also such compounds that are produced in the human or animal body by way of metabolism of a precursor compound, that is a compound of the Formula I and sub-formulae thereof may be a synthetically-produced compound or a metabolically-produced compound.

A suitable pharmaceutically acceptable pro-drug of a compound of the Formula I and sub-formulae thereof is one that is based on reasonable medical judgement as being suitable for administration to the human or animal body without undesirable pharmacological activities and without undue toxicity.

Various forms of pro-drug have been described, for example in the following documents:—
a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191 (1991);
d) H. Bundgaard, Advanced Drug Delivery Reviews, 8, 1-38 (1992);
e) H. Bundgaard, et al., Journal of Pharmaceutical Sciences, 77, 285 (1988);
f) N. Kakeya, et al., Chem. Pharm. Bull., 32, 692 (1984);
g) T. Higuchi and V. Stella, “Pro-Drugs as Novel Delivery Systems”, A.C.S. Symposium Series, Volume 14; and
h) E. Roche (editor), “Bioreversible Carriers in Drug Design”, Pergamon Press, 1987.

A suitable pharmaceutically acceptable pro-drug of a compound of the Formula I and sub-formulae thereof that possesses a carboxy group is, for example, an in vivo cleavable ester thereof. An in vivo cleavable ester of a compound of the Formula I containing a carboxy group is, for example, a pharmaceutically acceptable ester which is cleaved in the human or animal body to produce the parent acid. Suitable pharmaceutically acceptable esters for carboxy include C₁₋₆alkyl esters such as, but not limited to, methyl, ethyl and tert-butyl, C₁₋₆alkoxymethyl esters such as, but not limited to, methoxymethyl esters, C₁₋₆alkanoyloxymethyl esters such as, but not limited to, pivaloyloxymethyl esters, 3-phthalidyl esters, C₃₋₈cycloalkylcarbonyloxy-C₁₋₆alkyl esters such as, but not limited to, cyclopentylcarbonyloxymethyl and 1-cyclohexylcarbonyloxyethyl esters, 2-oxo-1,3-dioxolenylmethyl esters such as, but not limited to, 5-methyl-2-oxo-1,3-dioxolen-4-ylmethyl esters and C₁₋₆alkoxycarbonyloxy-C₁₋₆alkyl esters such as, but not limited to, methoxycarbonyloxymethyl and 1-methoxycarbonyloxyethyl esters.

A suitable pharmaceutically acceptable pro-drug of a compound of the Formula I and sub-formulae thereof that possesses a hydroxy group is, for example, an in vivo cleavable ester or ether thereof. An in vivo cleavable ester or ether of a compound of the Formula I and sub-formulae thereof containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as, but not limited to, phosphate esters (including phosphoramidic cyclic esters). Further suitable pharmaceutically acceptable ester forming groups for a hydroxy group include C₁₋₁₀alkanoyl groups such as, but not limited to, acetyl, benzoyl, phenylacetyl and substituted benzoyl and phenylacetyl groups, C₁₋₁₀alkoxycarbonyl groups such as, but not limited to, ethoxycarbonyl, N,N—(C₁₋₆)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁₋₄alkyl) piperazin-1-ylmethyl. Suitable pharmaceutically acceptable ether forming groups for a hydroxy group include α-acyloxyalkyl groups such as, but not limited to, acetoxymethyl and pivaloyloxymethyl groups.

A suitable pharmaceutically acceptable pro-drug of a compound of the Formula I and sub-formulae thereof that possesses a carboxy group is, for example, an in vivo cleavable amide thereof, for example an amide formed with an amine such as, but not limited to, ammonia, a C₁₋₄alkylamine such as, but not limited to, methylamine, a (C₁₋₄alkyl)₂amine such as, but not limited to, dimethylamine, N-ethyl-N-methylamine or diethylamine, a C₁₋₄alkoxy-C₂₋₄alkylamine such as, but not limited to, 2-methoxyethylamine, a phenyl-C₁₋₄alkylamine such as, but not limited to, benzylamine and amino acids such as, but not limited to, glycine or an ester thereof.

A suitable pharmaceutically acceptable pro-drug of a compound of the Formula I and sub-formulae thereof that possesses an amino group is, for example, an in vivo cleavable amide derivative thereof. Suitable pharmaceutically acceptable amides from an amino group include, for example an amide formed with C₁₋₁₀alkanoyl groups such as, but not limited to, an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁₋₄alkyl) piperazin-1-ylmethyl.

The in vivo effects of a compound of the Formula I and sub-formulae thereof may be exerted in part by one or more metabolites that are formed within the human or animal body after administration of a compound of the Formula I and sub-formulae thereof. As stated hereinbefore, the in vivo effects of a compound of the Formula I and sub-formulae thereof may also be exerted by way of metabolism of a precursor compound (a pro-drug).

### Synthesis

The compounds of the present invention can be prepared by any suitable technique known in the art. Particular methods for forming compounds of formula I defined herein are shown below and in the accompanying examples.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilised.

It will be appreciated that during the synthesis of the compounds of the invention in the processes defined herein, or during the synthesis of certain starting materials, it may be desirable to protect certain substituent groups to prevent their undesired reaction. The skilled chemist will appreciate when such protection is required, and how such protecting groups may be put in place, and later removed.

For Examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule.

Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as, but not limited to, acetyl, an alkoxycarbonyl group, for example a methoxycarbonyl, ethoxycarbonyl or tbutoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as, but not limited to, an alkali metal hydroxide, for example lithium or sodium hydroxide. Alternatively, an acyl group such as a tertbutoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium on carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively, an arylmethyl group such as a benzyl group may be removed, for example, by hydrogenation over a catalyst such as palladium on carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium on carbon.

Resins may also be used as a protecting group.

The methodology employed to synthesise a compound of formula (I) will vary depending on the nature of L, R_{L}, R_{R} and any substituent groups associated therewith. Suitable processes for their preparation are described further in the accompanying Examples.

Once a compound of formula (I) has been synthesised by any one of the processes defined herein, the processes may then further comprise one or more of the additional steps of:
(i) removing any residual protecting groups present;
(ii) converting the compound formula (I) into another compound of formula (I);
(iii) forming a pharmaceutically acceptable salt, hydrate or solvate of the compound of formula I; and/or
(iv) forming a prodrug of the compound of formula I.

An Example of (ii) above is when a compound of formula (I) is synthesised and then one or more of the groups of L, R_{L} and R_{R} may be further reacted to change the nature of the group and provide an alternative compound of formula (I).

The resultant compounds of formula (I) can be isolated and purified using techniques well known in the art.

According to a further aspect of the invention, there is provided a process for preparing a compound of formula (I) as hereinbefore described.

### Pharmaceutical Compositions

According to a further aspect of the invention there is provided a pharmaceutical composition which comprises a compound of the invention as defined hereinbefore, or a pharmaceutically acceptable salt, hydrate or solvate thereof, in association with a pharmaceutically acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

An effective amount of a compound of the present invention for use in therapy is an amount sufficient to treat or prevent a proliferative condition referred to herein, slow its progression and/or reduce the symptoms associated with the condition.

The amount of active ingredient that is combined with one or more excipients to produce a single dosage form will necessarily vary depending upon the individual treated and the particular route of administration. For example, a formulation intended for oral administration to humans will generally contain, for example, from 0.5 mg to 1.5 g of active agent (more suitably from 0.5 to 600 mg, for example from 1 to 200 mg) compounded with an appropriate and convenient amount of excipients which may vary from about 5 to about 98 percent by weight of the total composition.

The size of the dose for therapeutic or prophylactic purposes of a compound of the Formula I will naturally vary according to the nature and severity of the conditions, the age and sex of the animal or patient and the route of administration, according to well-known principles of medicine.

It is to be noted that dosages and dosing regimens may vary with the type and severity of the condition to be alleviated, and may include the administration of single or multiple doses, i.e. QD (once daily), BID (twice daily), etc., over a particular period of time (days or hours). It is to be further understood that for any particular subject or patient, specific dosage regimens may need to be adjusted over time according to the individual need and the professional judgment of the person administering or supervising the administration of the pharmaceutical compositions. For example, doses may be adjusted based on pharmacokinetic or pharmacodynamic parameters, which may include clinical effects such as toxic effects and/or laboratory values. Thus, the present application encompasses intra-patient dose-escalation as determined by the person skilled in the art. Procedures and processes for determining the appropriate dosage(s) and dosing regimen(s) are well-known in the relevant art and would readily be ascertained by the skilled artisan. As such, one of ordinary skill would readily appreciate and recognize that the dosage ranges set forth herein are exemplary only and are not intended to limit the scope or practice of the pharmaceutical compositions described herein.

In using a compound of the invention for therapeutic or prophylactic purposes it will generally be administered so that a daily dose in the range, for example, 0.1 mg/kg to 75 mg/kg body weight is received, given if required in divided doses. In general lower doses will be administered when a parenteral route is employed. Thus, for example, for intravenous or intraperitoneal administration, a dose in the range, for example, 0.1 mg/kg to 30 mg/kg body weight will generally be used. Similarly, for administration by inhalation, a dose in the range, for example, 0.05 mg/kg to 25 mg/kg body weight will be used.

For the compounds of the present invention, oral administration is particularly suitable. The compounds of the present invention may be formulated as a tablet, capsule or solution for oral administration. Suitably, the compound of the present invention is formulated in a unit dosage form (e.g. a tablet or capsule) for oral administration. Typically, unit dosage forms will contain about 0.5 mg to 1.5 g of a compound of this invention.

### Therapeutic Uses and Applications

The compounds of the present invention are potent inhibitors of Casein Kinase 2 alpha (CK2α). Data showing the CK2α inhibition for the exemplified compounds is presented in the accompanying example section.

The compounds of the present invention are designed to bind to the catalytic ATP site of CK2α (to drive potent enzyme inhibition) and the aD site (to drive high levels of selectivity over other kinases) [Brear et al, Chem Sci 2016].

Accordingly, the compounds of formula I are useful for the treatment and/or prevention of diseases and conditions in which CK2α activity is implicated, such as, for example, but not limited to, the treatment and/or prevention of proliferative disorders (e.g. cancer), viral infections, inflammation, diabetes, vascular and ischemic disorders, neurodegeneration and the regulation of circadian rhythm.

In another aspect, the present invention provides a compound of Formula I as defined herein, or a pharmaceutically acceptable salt, hydrate or solvate thereof, or a pharmaceutical composition as defined herein, for use in therapy.

In another aspect, the present invention provides a compound of Formula I as defined herein, or a pharmaceutically acceptable salt, hydrate or solvate thereof, or a pharmaceutical composition as defined herein, for use in the treatment of a disease or condition in which CK2α activity is implicated.

In another aspect, the present invention provides the use of a compound of Formula I as defined herein, or a pharmaceutically acceptable salt, hydrate or solvate thereof, in the manufacture of a medicament for use in the treatment of a disease or condition in which CK2α activity is implicated.

In another aspect, the present invention provides a method of treating a disease or condition in which CK2α activity is implicated, said method comprising administering to a subject in need thereof an effective amount of a compound of Formula I as defined herein, or a pharmaceutically acceptable salt, hydrate or solvate thereof, or a pharmaceutical composition as defined herein.

In another aspect, the present invention provides a compound of Formula I as defined herein, or a pharmaceutically acceptable salt, hydrate or solvate thereof, or a pharmaceutical composition as defined herein, for use in the treatment of a disease or condition associated with aberrant activity of CK2α.

In another aspect, the present invention provides the use of a compound of Formula I as defined herein, or a pharmaceutically acceptable salt, hydrate or solvate thereof, in the manufacture of a medicament for use in the treatment of a disease or condition associated with aberrant activity of CK2α.

In another aspect, the present invention provides a method of treating a disease or condition associated with aberrant activity of CK2α, said method comprising administering to a subject in need thereof an effective amount of a compound of Formula I as defined herein, or a pharmaceutically acceptable salt, hydrate or solvate thereof, or a pharmaceutical composition as defined herein.

In another aspect, the present invention provides a compound of Formula I as defined herein, or a pharmaceutically acceptable salt, hydrate or solvate thereof, or a pharmaceutical composition as defined herein, for use in the treatment of proliferative disorders (e.g. cancer or benign neoplasms), viral infections, an inflammatory disease or condition, diabetes, vascular and ischemic disorders, neurodegenerative disorders and/or the regulation of circadian rhythm.

In another aspect, the present invention provides the use of a compound of Formula I as defined herein, or a pharmaceutically acceptable salt, hydrate or solvate thereof, in the manufacture of a medicament for use in the treatment of proliferative disorders (e.g. cancer or benign neoplasms), viral infections, an inflammatory disease or condition, diabetes, vascular and ischemic disorders, neurodegenerative disorders and/or the regulation of circadian rhythm.

In another aspect, the present invention provides a method of treating a proliferative disorder (e.g. cancer or benign neoplasms), a viral infection, an inflammatory disease or condition, diabetes, vascular and ischemic disorders, neurodegenerative disorders and/or regulating cardiac rhythm, said method comprising administering to a subject in need thereof an effective amount of a compound of Formula I as defined herein, or a pharmaceutically acceptable salt, hydrate or solvate thereof, or a pharmaceutical composition as defined herein.

In another aspect, the present invention provides a compound of Formula I as defined herein, or a pharmaceutically acceptable salt, hydrate or solvate thereof, or a pharmaceutical composition as defined herein, for use in the treatment of a proliferative disorder.

In another aspect, the present invention provides the use of a compound of Formula I as defined herein, or a pharmaceutically acceptable salt, hydrate or solvate thereof, in the manufacture of a medicament for use in the treatment of a proliferative disorder (e.g. cancer or a benign neoplasms).

In another aspect, the present invention provides a method of treating a proliferative disorder (e.g. cancer or benign neoplasms), said method comprising administering to a subject in need thereof an effective amount of a compound of Formula I as defined herein, or a pharmaceutically acceptable salt, hydrate or solvate thereof, or a pharmaceutical composition as defined herein.

The terms “proliferative disorder” and “proliferative condition” are used interchangeably herein and pertain to an unwanted or uncontrolled cellular proliferation of excessive or abnormal cells which is undesired, such as, neoplastic or hyperplastic growth, whether in vitro or in vivo.

Examples of proliferative conditions include, but are not limited to, pre-malignant and malignant cellular proliferation, including but not limited to, cancers, psoriasis, bone diseases, fibroproliferative disorders (e.g. of connective tissues), and atherosclerosis. Any type of cell may be treated, including but not limited to, lung, colon, breast, ovarian, prostate, liver, pancreas, brain, blood and skin.

In certain aspects of the present invention, the proliferative disorder is cancer, suitably a cancer selected from lung, colon/colorectal, breast, ovarian, prostate, liver, pancreas, brain, blood, cholangiocarcinoma and skin cancer.

In a particular aspect of the invention, the proliferative disorder is colon/colorectal, cholangiocarcinoma, ovarian or prostate cancer.

In a particular aspect of the invention, the proliferative disorder is colorectal cancer.

In certain aspects of the present invention, the proliferative disorder is hematopoietic tumour, including: myelogenous and granulocytic leukemia (malignancy of the myeloid and granulocytic white blood cell series); lymphatic, lymphocytic, and lymphoblastic leukemia (malignancy of the lymphoid and lymphocytic blood cell series); polycythemia vera and erythremia (malignancy of various blood cell products, but with red cells predominating); and myelofibrosis.

A benign neoplasm may be, for example, hemangiomas, hepatocellular adenoma, cavernous haemangioma, focal nodular hyperplasia, acoustic neuromas, neurofibroma, bile duct adenoma, bile duct cystanoma, fibroma, lipomas, leiomyomas, mesotheliomas, teratomas, myxomas, nodular regenerative hyperplasia, trachomas, pyogenic granulomas, moles, uterine fibroids, thyroid adenomas, adrenocortical adenomas or pituitary adenomas. The benign neoplasm may be endometrial implants or a keratocystic odontogenic tumor.

In another aspect, the present invention provides a compound of Formula I as defined herein, or a pharmaceutically acceptable salt, hydrate or solvate thereof, or a pharmaceutical composition as defined herein, for use in the treatment of a cancer.

In another aspect, the present invention the use of a compound of Formula I as defined herein, or a pharmaceutically acceptable salt, hydrate or solvate thereof, in the manufacture of a medicament for use in the treatment of a cancer.

In another aspect, the present invention provides a method of treating cancer, said method comprising administering to a subject in need thereof an effective amount of a compound of Formula I as defined herein, or a pharmaceutically acceptable salt, hydrate or solvate thereof, or a pharmaceutical composition as defined herein.

The cancer may be non-metastatic or metastatic and which may be a solid tumour or a haematological (“liquid”) cancer. The cancer may, for example, be selected from:
(1) Carcinoma, including for example tumours derived from stratified squamous epithelia (squamous cell carcinomas) and tumours arising within organs or glands (adenocarcinomas). Examples include breast, colon, lung, prostate, ovary, esophageal carcinoma (including, but not limited to, esophageal adenocarcinoma and squamous cell carcinoma), basal-like breast carcinoma, basal cell carcinoma (a form of skin cancer), squamous cell carcinoma (various tissues), head and neck carcinoma (including, but not limited to, squamous cell carcinomas), stomach carcinoma (including, but not limited to, stomach adenocarcinoma, gastrointestinal stromal tumor), signet ring cell carcinoma, bladder carcinoma (including transitional cell carcinoma (a malignant neoplasm of the bladder)), bronchogenic carcinoma, colorectal carcinoma (including, but not limited to, colon carcinoma and rectal carcinoma), anal carcinoma, gastric carcinoma, lung carcinoma (including but not limited to small cell carcinoma (SCLC) and non-small cell carcinoma of the lung (NSCLC), lung adenocarcinoma, squamous cell carcinoma, large cell carcinoma, bronchioloalveolar carcinoma, and mesothelioma), neuroendocrine tumors (including but not limited to carcinoids of the gastrointestinal tract, breast, and other organs), adrenocortical carcinoma, thyroid carcinoma, pancreatic carcinoma (including, but not limited to, pancreatic ductal adenocarcinoma, pancreatic adenocarcinoma, acinar cell carcinoma, intraductal papillary mucinous neoplasm with invasive carcinoma, mucinous cystic neoplasm with invasive carcinoma, islet cell carcinoma and neuroendocrine tumors), breast carcinoma (including, but not limited to, ductal carcinoma, lobular carcinoma, inflammatory breast cancer, clear cell carcinoma, mucinous carcinoma), ovarian carcinoma (including, but not limited to, ovarian epithelial carcinoma or surface epithelial-stromal tumor including serous tumor, endometrioid tumor and mucinous cystadenocarcinoma, sex-cord-stromal tumor), liver and bile duct carcinoma (including, but not limited to, hepatocellular carcinoma, cholangiocarcinoma and hemangioma), prostate carcinoma, adenocarcinoma, brain tumours (including, but not limited to glioma, glioblastoma and medulloblastoma), germ cell tumors, sweat gland carcinoma, sebaceous gland carcinoma, papillary carcinoma, papillary adenocarcinoma, cystadenocarcinoma, kidney carcinoma (including, but not limited to, renal cell carcinoma, clear cell carcinoma and Wilm's tumor), medullary carcinoma, ductal carcinoma in situ or bile duct carcinoma, choriocarcinoma, seminoma, embryonal carcinoma, cervical carcinoma, uterine carcinoma (including, but not limited to, endometrial adenocarcinoma, uterine papillary serous carcinoma, uterine clear-cell carcinoma, uterine sarcomas and leiomyosarcomas, mixed mullerian tumors), testicular carcinoma, osteogenic carcinoma, epithelial carcinoma, sarcomatoid carcinoma, nasopharyngeal carcinoma, laryngeal carcinoma; oral and oropharyngeal squamous carcinoma;
(2) Sarcomas, including: osteosarcoma and osteogenic sarcoma (bone); chondrosarcoma (cartilage); leiomyosarcoma (smooth muscle); rhabdomyosarcoma (skeletal muscle); mesothelial sarcoma and mesothelioma (membranous lining of body cavities); fibrosarcoma (fibrous tissue); angiosarcoma and hemangioendothelioma (blood vessels); liposarcoma (adipose tissue); glioma and astrocytoma (neurogenic connective tissue found in the brain); myxosarcoma (primitive embryonic connective tissue); chordoma, endotheliosarcoma, lymphangiosarcoma, lymphangioendotheliosarcoma, synovioma, Ewing's sarcoma, mesenchymous and mixed mesodermal tumor (mixed connective tissue types) and other soft tissue sarcomas;
(3) Myeloma and multiple myeloma;
(4) Hematopoietic tumours, including: myelogenous and granulocytic leukemia (malignancy of the myeloid and granulocytic white blood cell series); lymphatic, lymphocytic, and lymphoblastic leukemia (malignancy of the lymphoid and lymphocytic blood cell series); polycythemia vera and erythremia (malignancy of various blood cell products, but with red cells predominating); myelofibrosis.
(5) Lymphomas, including: Hodgkin and Non-Hodgkin lymphomas;
(6) Solid tumors of the nervous system including medulloblastoma, craniopharyngioma, ependymoma, pinealoma, hemangioblastoma, acoustic neuroma, oligodendroglioma, meningioma, neuroblastoma and schwannoma;
(7) Melanoma, uveal melanoma and retinoblastoma; and
(8) Mixed Types, including, e.g., adenosquamous carcinoma, mixed mesodermal tumor, carcinosarcoma or teratocarcinoma.

Suitably, a compound of the invention, or a pharmaceutically acceptable salt thereof may be for use in the treatment of a cancer selected from cancer selected from, colon/colorectal cancer, cholangiocarcinoma, gastric cancer, skin cancer (e.g. basal cell carcinoma), ovarian, prostate, breast cancer, liver cancer, pancreatic cancer, brain cancer, blood cancers (leukaemia's, myelomas), bladder cancer, bone cancer, head and neck cancer, renal cancer and lung cancer.

More suitably, the cancer is selected from colon/colorectal cancer, prostate cancer, ovarian cancer, basal cell carcinoma or cholangiocarcinoma.

In a particular aspect of the present invention, the cancer is basal cell carcinoma.

In a further aspect of the present invention, the cancer is colorectal cancer.

In a further aspect of the present invention, the cancer is cholangiocarcinoma.

In a further aspect of the present invention, the cancer is prostate cancer.

In a further aspect of the present invention, the cancer is ovarian cancer.

In another aspect of the present invention, the cancer is a hematopoietic tumour.

It is hypothesised that the compounds of the present invention will be particularly suited to the treatment of wnt pathway driven cancers, e.g. wnt pathway mutated colorectal cancer or cholangiocarcinoma (Di Maira et al, 2019).

In addition to CK2α having a very well characterized function in wnt pathway activity, it also plays a role in other key cellular pathways known to be upregulated in cancer, such as, but not limited to, the DNA damage response (Ruzzene & Pinna, 2010; Montenarh, Transl. Cancer Res 2016). Thus, the compounds of the present invention may have a further use in the treatment of PARP insensitive tumors in prostate/ovarian cancer.

CK2α has also recently been identified as a key host protein required for viral replication (e.g. in SARS-CoV2) and as such could represent an antiviral treatment (Gordon et al. Nature 2020).

Thus, in another aspect, the present invention provides a compound of Formula I as defined herein, or a pharmaceutically acceptable salt, hydrate or solvate thereof, or a pharmaceutical composition as defined herein, for use in the treatment of a viral infection.

In another aspect, the present invention provides the use of a compound of Formula I as defined herein, or a pharmaceutically acceptable salt, hydrate or solvate thereof, in the manufacture of a medicament for use in the treatment of a viral infection.

In another aspect, the present invention provides a method of treating a viral infection, said method comprising administering to a subject in need thereof an effective amount of a compound of Formula I as defined herein, or a pharmaceutically acceptable salt, hydrate or solvate thereof, or a pharmaceutical composition as defined herein.

Suitably, the virus is a coronavirus, e.g. SARS-CoV2.

### Routes of Administration

The compounds of the invention or pharmaceutical compositions comprising these compounds may be administered to a subject by any convenient route of administration, whether systemically/peripherally or topically (i.e., at the site of desired action).

Routes of administration include, but are not limited to, oral (e.g. by ingestion); buccal; sublingual; transdermal (e.g. by a patch, plaster, etc.); transmucosal (e.g. by a patch, plaster, etc.); intranasal (e.g. by nasal spray); ocular (e.g. by eye drops, eye ointment etc.); pulmonary (e.g. by inhalation or insufflation therapy, for example via an aerosol, for example by the nose or mouth); rectal (e.g. by suppository or enema); vaginal (e.g. by pessary); parental, for example by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir dosage form, for example subcutaneously or intramuscularly.

The compounds of the present invention are particularly suitable for oral administration.

### Combination Therapies

The compounds of the invention and salts, solvates thereof defined hereinbefore may be applied as a sole therapy or may involve, in addition to the compound of the invention, one or more additional therapeutic agents, e.g. an anti-tumour agent.

In the context of cancer treatment, in addition to the compound of the invention therapy may involve conventional surgery or radiotherapy or chemotherapy. Such chemotherapy may include one or more of the following categories of anti-tumour agents:—
other antiproliferative/antineoplastic drugs and combinations thereof, as used in medical oncology, such as, but not limited to, alkylating agents (for example cisplatin, oxaliplatin, carboplatin, cyclophosphamide, nitrogen mustard, melphalan, chlorambucil, busulphan, temozolamide and nitrosoureas); antimetabolites (for example gemcitabine and antifolates such as, but not limited to, fluoropyrimidines like 5-fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);
cytostatic agents such as, but not limited to, antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as, but not limited to, finasteride;
anti-invasion agents [for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341), N-(2-chloro-6-methylphenyl)-2-{6-[4-(2-hydroxyethyl) piperazin-1-yl]-2-methylpyrimidin-4-ylamino}thiazole-5-carboxamide (dasatinib, BMS-354825; J. Med. Chem., 2004, 47, 6658-6661) and bosutinib (SKI-606), and metalloproteinase inhibitors like marimastat, inhibitors of urokinase plasminogen activator receptor function or antibodies to Heparanase];
inhibitors of growth factor function: for example such inhibitors include growth factor antibodies and growth factor receptor antibodies (for example the anti-erbB2 antibody trastuzumab [Herceptin™], the anti-EGFR antibody panitumumab, the anti-erbB1 antibody cetuximab [Erbitux, C225] and any growth factor or growth factor receptor antibodies disclosed by Stern et al. (Critical reviews in oncology/haematology, 2005, Vol. 54, pp11-29); such inhibitors also include tyrosine kinase inhibitors, for example inhibitors of the epidermal growth factor family (for example EGFR family tyrosine kinase inhibitors such as, but not limited to, N-(3-chloro-4-fluorophenyl)-7-methoxy-6-(3-morpholinopropoxy) quinazolin-4-amine (gefitinib, ZD1839), N-(3-ethynylphenyl)-6,7-bis(2-methoxyethoxy) quinazolin-4-amine (erlotinib, OSI-774) and 6-acrylamido-N-(3-chloro-4-fluorophenyl)-7-(3-morpholinopropoxy)-quinazolin-4-amine (CI 1033), erbB2 tyrosine kinase inhibitors such as, but not limited to, lapatinib); inhibitors of the hepatocyte growth factor family; inhibitors of the insulin growth factor family; inhibitors of the platelet-derived growth factor family such as, but not limited to, imatinib and/or nilotinib (AMN107); inhibitors of serine/threonine kinases (for example Ras/Raf signalling inhibitors such as, but not limited to, farnesyl transferase inhibitors, for example sorafenib (BAY 43-9006), tipifarnib (R115777) and lonafarnib (SCH66336)), inhibitors of cell signalling through MEK and/or AKT kinases, c-kit inhibitors, abl kinase inhibitors, PI3 kinase inhibitors, Plt3 kinase inhibitors, CSF-1R kinase inhibitors, IGF receptor (insulin-like growth factor) kinase inhibitors; aurora kinase inhibitors (for example AZD1152, PH739358, VX-680, MLN8054, R₇₆₃, MP235, MP529, VX-528 AND AX39459) and cyclin dependent kinase inhibitors such as, but not limited to, CDK2 and/or CDK4 inhibitors;
antiangiogenic agents such as, but not limited to, those which inhibit the effects of vascular endothelial growth factor, [for example the anti-vascular endothelial cell growth factor antibody bevacizumab (Avastin™) and for example, a VEGF receptor tyrosine kinase inhibitor such as, but not limited to, vandetanib (ZD6474), vatalanib (PTK787), sunitinib (SU11248), axitinib (AG-013736), pazopanib (GW 786034) and 4-(4-fluoro-2-methylindol-5-yloxy)-6-methoxy-7-(3-pyrrolidin-1-ylpropoxy)quinazoline (AZD2171; Example 240 within WO 00/47212), compounds such as, but not limited to, those disclosed in International Patent Applications WO97/22596, WO 97/30035, WO 97/32856 and WO 98/13354 and compounds that work by other mechanisms (for example linomide, inhibitors of integrin αvβ3 function and angiostatin)];
vascular damaging agents such as, but not limited to, Combretastatin A4 and compounds disclosed in International Patent Applications WO 99/02166, WO 00/40529, WO 00/41669,

### WO 01/92224, WO 02/04434 and WO 02/08213;

an endothelin receptor antagonist, for example zibotentan (ZD4054) or atrasentan;
antisense therapies, for example those which are directed to the targets listed above, such as, but not limited to, ISIS 2503, an anti-ras antisense;
gene therapy approaches, including for example approaches to replace aberrant genes such as, but not limited to, aberrant p53 or aberrant BRCA1 or BRCA2, GDEPT (gene-directed enzyme pro-drug therapy) approaches such as, but not limited to, those using cytosine deaminase, thymidine kinase or a bacterial nitroreductase enzyme and approaches to increase patient tolerance to chemotherapy or radiotherapy such as multi-drug resistance gene therapy; and
immunotherapy approaches, including for example ex-vivo and in-vivo approaches to increase the immunogenicity of patient tumour cells, such as, but not limited to, transfection with cytokines such as interleukin 2, interleukin 4 or granulocyte-macrophage colony stimulating factor, approaches to decrease T-cell anergy, approaches using transfected immune cells such as, but not limited to, cytokine-transfected dendritic cells, approaches using cytokine-transfected tumour cell lines and approaches using anti-idiotypic antibodies.

In a particular embodiment, the antiproliferative treatment defined hereinbefore may involve, in addition to the compound of the invention, conventional surgery or radiotherapy or chemotherapy.

In a further particular embodiment, the antiproliferative treatment defined hereinbefore may involve, in addition to the compound of the invention, standard chemotherapy for the cancer concerned.

In a particular embodiment, the antiproliferative treatment defined hereinbefore may involve, in addition to the compound of the invention, therapy with K-ras inhibitors and/or DNA damage repair inhibitors (e.g. PARP inhibitors).

Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

According to this aspect of the invention there is provided a combination for use in the treatment of a cancer (for example a cancer involving a solid tumour) comprising a compound of the invention as defined hereinbefore, or a pharmaceutically acceptable salt, hydrate or solvate thereof, and another anti-tumour agent.

According to this aspect of the invention there is provided a combination for use in the treatment of a proliferative condition, such as, but not limited to, cancer (for example a cancer involving a solid tumour), comprising a compound of the invention as defined hereinbefore, or a pharmaceutically acceptable salt, hydrate or solvate thereof, and any one of the anti-tumour agents listed herein above.

In a further aspect of the invention there is provided a compound of the invention or a pharmaceutically acceptable salt, hydrate or solvate thereof, for use in the treatment of cancer in combination with another anti-tumour agent, optionally selected from one listed herein above.

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination. In one embodiment, a combination refers to a combination product.

According to a further aspect of the invention there is provided a pharmaceutical composition which comprises a compound of the invention, or a pharmaceutically acceptable salt, hydrate or solvate thereof, in combination with an anti-tumour agent (optionally selected from one listed herein above), in association with a pharmaceutically acceptable diluent or carrier.

### Biological Activity

The biological assay described in the example section (Biological Assay 1) may be used to measure the pharmacological effects of the compounds of the present invention.

Although the pharmacological properties of the compounds of formula I vary with structural change, as expected, the compounds of the invention were found to be active in the assays described in Biological Assay 1. In general, the compounds of the invention demonstrate an IC₅₀ of 500 nM or less in the assay described in Biological Assay 1, with preferred compounds of the invention demonstrating an IC₅₀ of 100 nM or less and the most preferred compounds of the invention demonstrating an IC₅₀ of 30 nM or less.

Compounds of the invention may also show activity in Assay 3 described in the accompanying Biological Assay section.

### EXAMPLES

The invention will now be illustrated, but not limited, by reference to the specific embodiments described in the following examples. Compounds are named using conventional IUPAC nomenclature, or as named by the chemical supplier.

The following synthetic procedures are provided for illustration of the methods used; for a given preparation or step the precursor used may not necessarily derive from the individual batch synthesized according to the step in the description given.

### Analytical Methods (AM)

Where examples and preparations cite analytical data, the following analytical methods were used unless otherwise specified.

All LCMS spectra were obtained by using one of the below methods.
Method 1 (AM1): (5-95 A-B_1.5 min_220 & 254 nm): Instrument: Agilent 1100\G1956A; Column: Kinetex@ 5 um EVO C18 30×2.1 mm×5 μm; Run Time: 1.5 min; Solvents: A) 0.0375% TFA in H₂O (v/v), B) 0.01875% TFA in acetonitrile (v/v). The gradient runs with 5% B; Gradient: 5-95% B with A, 0.8 min; hold at 95% B to 1.2 min; 5% B at 1.21 min and hold at 5% B to1.5 min @ 1.5 mL/min, 50° C.
Method 2 (AM2): (5-95 A-B_1.5 min_220 & 254 nm): Instrument: Agilent 1200\G6110A; Column: Kinetex@ 5 um EVO C18 30×2.1 mm×5 μm; Run Time: 1.5 min; Solvents: A) 0.0375% TFA in H₂O (v/v), B) 0.01875% TFA in acetonitrile (v/v). The gradient runs with 5% B; Gradient: 5-95% B with A, 0.8 min; hold at 95% B to 1.2 min; 5% B at 1.21 min and hold at 5% B to 1.5 min @ 1.5 mL/min, 50° C.
Method 3 (AM3): (5-95 A-B_1.55 min_220 & 254 nm): Instrument: SHIMADZU LCMS-2020; Column: Kinetex EVO C18 30×2.1 mm×5 μm; Run Time: 1.55 min; Solvents: A) 0.0375% TFA in H₂O (v/v), B) 0.01875% TFA in acetonitrile (v/v). The gradient runs with 5% B; Gradient: 5-95% B with A, 0.8 min; hold at 95% B to 1.2 min; 5% B at 1.21 min and hold at 5% B to 1.55 min @ 1.5 mL/min, 50° C.
Method 4 (AM4): (5-95 A-B_1.5 min_220 & 254 nm): Instrument: Agilent 1200 LC/G1956A MSD; Column: Kinetex EVO C18 30×2.1 mm×5 μm; Run Time: 1.5 min; Solvents: A) 0.0375% TFA in H₂O (v/v), B) 0.01875% TFA in acetonitrile (v/v). The gradient runs with 5% B; Gradient: 5-95% B with A, 0.8 min; hold at 95% B to 1.2 min; 5% B at 1.21 min and hold at 5% B to 1.5 min @ 1.5 mL/min, 50° C.
Method 5 (AM5): (0-60 A-B_1.55 min_220 & 254 nm): Instrument: SHIMADZU LCMS-2020; Column: Kinetex EVO C18 30×2.1 mm×5 μm; Run Time: 1.55 min; Solvents: A) 0.0375% TFA in H₂O (v/v), B) 0.01875% TFA in ACN (v/v). The gradient runs with 0% B; Gradient: 0-60% B with A, 0.8 min; hold at 60% B to 1.20 min; 0% B at 1.21 min and hold at 0% B to 1.55 min @ 1.5 mL/min, 50° C.
Method 6 (AM6): (0-60 C-D_2.20 min_220 & 254 nm): Instrument: SHIMADZU LCMS-2020; Column: Kinetex EVO C18 30×2.1 mm×5 μm; Run Time: 2.20 min; Solvents: A) 0.025% NH₃·H₂O in H₂O (v/v), B) acetonitrile. The gradient runs with 0% B; Gradient: 0-60% B with A, 1.2 min; hold at 60% B to 1.6 min; 0% B at 1.61 min and hold at 0% B to 2.2 min @ 1.5 mL/min, 40° C.
Method 7 (AM7): (5-95 C-D_1.5 min_R_220&254_POS): Instrument: SHIMADZU LCMS-2020; Column: Kinetex EVO C18 30×2.1 mm×5 μm; Run Time: 1.5 min; Solvents A) 0.025% NH₃·H₂O in H₂O (v/v) B) Acetonitrile. The gradient runs with 5% B. Gradient: 5-95% B with A 0.8 min, hold at 95% B to 1.2 min; 5% B at 1.21 min and hold at 5% B to 1.5 min @ 1.5 ml/min, 40° C.
Method 8 (AM8): (10-80 C-D_2.00 min_220 & 254 nm): Instrument: Agilent 1200\G6110A; Column: ACE Excel 5 C18 30×2.1 mm×5 μm; Run Time: 2.00 min; Solvents: A) 0.025% NH₃·H₂O in H₂O (v/v), B) Acetonitrile (v/v). The gradient runs with 10% B; Gradient: 10-80% B with A, 1.2 min; hold at 80% B to 1.6 min; 10% B at 1.61 min and hold at 10% B to 2.00 min @ 1.0 mL/min, 40° C.
Method 9 (AM9): (10-80 A-B_7 min_220 & 254 nm): Instrument: SHIMADZU LCMS-2020; Column: AB: Xtimate C18 30×2.1 mm×3 μm; Run Time: 7.0 min; Solvents: A) 0.0375% TFA in H₂O (v/v), B) 0.01875% TFA in acetonitrile (v/v). The gradient runs with 10% B; Gradient: 10-80% B with A, 6.5 min; hold at 80% B to 7 min; 10% B at 6.5 min and hold at 10% B to 7 min @ 1.5 mL/min, 50° C.
¹H NMR spectra were acquired on a Bruker Avance III spectrometer at 400 MHz using residual undeuterated solvent as reference. The spectra were processed using interpretation software ACD Spectrus processor or equivalent software.

### Purification Methods (PM)

### Chromatography

| Purification | | | Eluent |
|---|---|---|---|
| method | Column | Eluent | Ratio |
| PM1 | SiO₂ | petroleum ether | 1 |
| PM2 | SiO₂ | petroleum ether:ethyl acetate | 1:1 |
| PM3 | SiO₂ | petroleum ether:ethyl acetate | 2:1 |
| PM4 | SiO₂ | petroleum ether:ethyl acetate | 3:1 |
| PM5 | SiO₂ | petroleum ether:ethyl acetate | 4:1 |
| PM6 | SiO₂ | petroleum ether:ethyl acetate | 5:1 |
| PM7 | SiO₂ | petroleum ether:ethyl acetate | 8:1 |
| PM8 | SiO₂ | petroleum ether:ethyl acetate | 10:1 |
| PM9 | SiO₂ | petroleum ether:ethyl acetate | 12:1 |
| PM10 | SiO₂ | petroleum ether:ethyl acetate | 15:1 |
| PM11 | SiO₂ | petroleum ether:ethyl acetate | 20:1 |
| PM12 | SiO₂ | petroleum ether:ethyl acetate | 30:1 |
| PM13 | SiO₂ | petroleum ether:ethyl acetate | 40:1 |
| PM14 | SiO₂ | petroleum ether:ethyl acetate | 50:1 |
| PM15 | SiO₂ | petroleum ether:ethyl acetate | 100:1 |
| PM16 | SiO₂ | DCM:Methanol | 100:1 to 10:1 |
| PM17 | SiO₂ | petroleum ether:ethyl acetate | 8:1 to 2:1 |
| PM18 | SiO₂ | petroleum ether:ethyl acetate | 100:1 to 30:1 |
| PM19 | SiO₂ | petroleum ether:ethyl acetate | 10:1 to 3:1 |
| PM20 | SiO₂ | petroleum ether:ethyl acetate | 10:1 to 8:1 |
| PM21 | SiO₂ | ethyl acetate | 1 |

Reverse-phase HPLC conditions

| Purification | | Mobile | |
|---|---|---|---|
| method | Column | phase | Gradient |
| 22 | Phenomenex Gemini-NX C18 | water (0.1% TFA)- | 20%-55%, |
| | 75 × 30 mm × 3 mm | ACN | 10 min |
| 23 | Phenomenex Gemini-NX C18 | water (0.1% | 20%-55%, |
| | 75 × 30 mm × 3 mm | NH₃•H₂O)-ACN] | 10 min |
| 24 | Phenomenex luna C18 150 × 25 | water (0.075% TFA)- | 30%-60%, 9 |
| | mm × 10 mm | ACN | min |
| 25 | Phenomenex Gemini-NX C18 | water (0.1% FA)- | 20%-55%, |
| | 75 × 30 mm × 3 mm | ACN | 15 min |
| 26 | Phenomenex Gemini-NX C18 | water (0.1% TFA)- | 20%-55%, |
| | 75 × 30 mm × 3 mm | ACN | 15 min |
| 27 | Phenomenex Gemini-NX C18 | water (0.1% TFA)- | 20%-55%, |
| | 75 × 30 mm × 3 mm | ACN | 20 min |
| 28 | Phenomenex Gemini-NX C18 | water (0.1% FA)- | 20%-80%, |
| | 75 × 30 mm × 3 mm | ACN | 20 min |
| 29 | Phenomenex Gemini-NX C18 | water (0.1% HCl)- | 20%-80%, |
| | 75 × 30 mm × 3 mm | ACN | 20 min |
| 30 | Phenomenex Gemini-NX C18 | water (0.1% TFA)- | 20%-80%, |
| | 75 × 30 mm × 3 mm | ACN | 20 min |
| 31 | Phenomenex Synergi C18 150 × | water (0.1% TFA)- | 18%-38%, |
| | 25 mm × 10 mm | ACN | 20 min |
| 32 | Phenomenex Synergi C18 150 × | water (0.1% TFA)- | 18%-38%, |
| | 25 mm × 10 mm | ACN | 12 min |
| 33 | Phenomenex Synergi C18 150 × | water (0.05% HCl)- | 55%-75%, |
| | 25 mm × 10 mm | ACN | 12 min |
| 34 | Phenomenex luna C18 150 × 25 | water (0.1% TFA)- | 21%-51%, |
| | mm × 10 mm | ACN | 10 min |
| 35 | Phenomenex luna C18 150 × 25 | water (0.075% TFA)- | 28%-58%, 9 |
| | mm × 10 mm | ACN | min |
| 36 | Phenomenex luna C18 150 × 25 | water (0.1% TFA)- | 38%-68%, |
| | mm × 10 mm | ACN | 11 min |
| 37 | Phenomenex Synergi C18 150 × | water (0.1% TFA)- | 20%-50%, |
| | 25 mm × 10 mm | ACN | 10 min |
| 38 | Phenomenex luna C18 200 × 40 | water (0.1% TFA)- | 25%-55%, |
| | mm × 10 mm | ACN | 10 min |
| 39 | Phenomenex Synergi C18 150 × | water-ACN | 10%-90%, |
| | 25 mm × 10 mm | | 20 min |
| 40 | Phenomenex Synergi C18 150 × | water (0.1% TFA)- | 10%-90%, |
| | 25 mm × 10 mm | ACN | 20 min |
| 41 | Phenomenex luna C18 150 × 25 | water (0.075% TFA)- | 20%-50%, |
| | mm × 10 mm | ACN | 2 min |
| 42 | Phenomenex luna C18 150 × 25 | water (0.1% TFA)- | 5%-35%, |
| | mm × 10 mm | ACN | 10 min |
| 43 | Phenomenex Gemini 150 × 25 | water (0.225% FA)- | 27%-54%, |
| | mm × 10 mm | ACN | 10 min |
| 44 | Phenomenex Synergi C18 150 × | water (0.1% TFA)- | 15%-45%, |
| | 25 mm × 10 mm | ACN | 10 min |
| 45 | Phenomenex luna C18 150 × 25 | water (0.075% TFA)- | 15%-45%, |
| | mm × 10 mm | ACN | 9 min |
| 46 | Phenomenex Synergi C18 150 × | water (0.1% FA)- | 10%-90%, |
| | 25 mm × 10 mm | ACN | 20 min |
| 47 | Phenomenex luna C18 150 × 25 | water (0.1% TFA)- | 18%-48%, |
| | mm × 10 mm | ACN | 10 min |
| 48 | Phenomenex Synergi C18 150 × | water (0.1% HCl)- | 10%-90%, |
| | 25 mm × 10 mm | ACN | 20 min |
| 49 | Phenomenex luna C18 150 × 25 | water (0.1% TFA)- | 35%-65%, |
| | mm × 10 mm | ACN | 10 min |
| 50 | Phenomenex Synergi C18 150 × | water (0.1% TFA)- | 20%-45%, |
| | 25 mm × 10 mm | ACN | 9 min |
| 51 | Phenomenex luna C18 150 × 25 | water (0.075% TFA)- | 15%-45%, |
| | mm × 10 mm | ACN | 2 min |
| 52 | Phenomenex luna C18 150 × 25 | water (0.075% TFA)- | 12%-42%, |
| | mm × 10 mm | ACN | 2 min |
| 53 | Phenomenex Synergi C18 150 × | water (0.1% TFA)- | 18%-48%, |
| | 25 mm × 10 mm | ACN | 13 min |
| 54 | Phenomenex Synergi C18 150 × | water (0.1% TFA)- | 35%-85%, |
| | 25 mm × 10 mm | ACN | 10 min |
| 55 | Phenomenex luna C18 250 × 50 | water (0.225% FA)- | 55%-80%, |
| | mm × 10 mm | ACN | 20 min |
| 56 | Phenomenex Synergi C18 150 × | water (0.1% TFA)- | 15%-45%, |
| | 25 mm × 10 mm | ACN | 13 min |
| 57 | Phenomenex luna C18 200 × 40 | water (0.1% TFA)- | 52%-82%, |
| | mm × 10 mm | ACN | 10 min |
| 58 | Phenomenex Synergi Max-RP | water (0.225% FA)- | 20%-50%, |
| | 150 × 50 mm × 10 mm | ACN | 10 min |
| 59 | Phenomenex luna C18 200 × 40 | water (0.1% TFA)- | 35%-65%, |
| | mm × 10 mm | ACN | 10 min |
| 60 | Phenomenex Synergi Max-RP | water (0.225% FA)- | 25%-55%, |
| | 150 × 50 mm × 10 mm | ACN | 6 min |
| 61 | Phenomenex Synergi C18 150 × | water (0.1% | 10%-90%, |
| | 25 mm × 10 mm | ammonium | 20 min |
| | | hydroxide)-ACN | |
| 62 | Phenomenex luna C18 150 × 25 | water (0.075% TFA)- | 42%-72%, |
| | mm × 10 mm | ACN | 9 min |
| 63 | Phenomenex luna C18 150 × 25 | water (0.075% TFA)- | 2%-32%, |
| | mm × 10 mm | ACN | 9 min |
| 64 | Phenomenex luna C18 250 × 50 | water (0.075% TFA)- | 20%-50%, |
| | mm × 10 mm | ACN | 9 min |
| 65 | Phenomenex Synergi C18 150 × | water (0.05% HCl)- | 24%-44%, |
| | 25 mm × 10 mm | ACN | 10 min |
| 66 | Phenomenex Synergi C18 150 × | water (0.225% FA)- | 18%-48%, |
| | 25 mm × 10 mm | ACN | 10 min |
| 67 | Phenomenex Synergi C18 150 × | water (0.1% TFA)- | 40%-60%, |
| | 25 mm × 10 mm | ACN | 10 min |
| 68 | Phenomenex Synergi C18 150 × | water (0.1% TFA)- | 42%-62%, |
| | 25 mm × 10 mm | ACN | 10 min |
| 69 | Phenomenex Synergi C18 150 × | water (0.1% TFA)- | 24%-54%, |
| | 25 mm × 10 mm | ACN | 10 min |
| 70 | Phenomenex luna C18 250 × 50 | water (0.075% TFA)- | 35%-65%, 9 |
| | mm × 10 mm | ACN | min |
| 71 | Phenomenex luna C18 250 × 50 | water (0.075% TFA)- | 22%-52%, 9 |
| | mm × 10 mm | ACN | min |
| 72 | Phenomenex Synergi C18 150 × | water (0.1% TFA)- | 18%-48%, |
| | 25 mm × 10 mm | ACN | 10 min |
| 73 | Phenomenex luna C18 150 × 25 | water (0.075% TFA)- | 15%-45%, |
| | mm × 10 mm | ACN | 10 min |
| 74 | Phenomenex Synergi C18 150 × | water (0.1% | 10%-90%, |
| | 25 mm × 10 mm | NH₃•H₂O)-ACN] | 20 min |
| 75 | Phenomenex luna C18 250 × 50 | water (0.1% TFA)- | 22%-52%, |
| | mm × 10 mm | ACN | 10 min |
| 76 | Phenomenex Synergi Max-RP | water (0.1% TFA)- | 22%-52%, |
| | 250 × 50 mm × 10 mm | ACN | 10 min |
| 77 | Phenomenex luna C18 150 × 25 | water (0.1% TFA)- | 38%-68%, |
| | mm × 10 mm | ACN | 10 min |
| 78 | Phenomenex Gemini-NX C18 | water (0.1% TFA)- | 25%-35%, |
| | 75 × 30 mm × 3 mm | ACN | 7 min |
| 79 | Phenomenex luna C18 150 × 40 | water (0.1% TFA)- | 46%-76%, |
| | mm × 15 mm | ACN | 10 min |
| 80 | Phenomenex luna C18 200 × 40 | water (0.1% TFA)- | 32%-62%, |
| | mm × 10 mm | ACN | 11 min |
| 81 | Phenomenex Gemini-NX C18 | water (0.1% TFA)- | 45%-55%, |
| | 75 × 30 mm × 3 mm | ACN | 7 min |
| 82 | Phenomenex Gemini-NX C18 | water (0.1% | 5%-35%, 7 |
| | 75 × 30 mm × 3 mm | ammonium | min |
| | | hydroxide)-ACN | |
| 83 | Phenomenex Gemini-NX C18 | water (0.1% | 38%-68%, |
| | 75 × 30 mm × 3 mm | ammonium | 10 min |
| | | hydroxide)-ACN | |
| 84 | Phenomenex Gemini-NX C18 | water (0.1% TFA)- | 28%-38%, |
| | 75 × 30 mm × 3 mm | ACN | 7 min |
| 85 | Phenomenex luna C18 150 × 40 | water (0.1% TFA)- | 36%-66%, |
| | mm × 15 mm | ACN | 10 min |
| 86 | Phenomenex Synergi Max-RP | water (0.05% HCl)- | 15%-45%, |
| | 250 × 50 mm × 10 mm | ACN | 30 min |
| 87 | Phenomenex Synergi Max-RP | water (0.05% HCl)- | 17%-47%, |
| | 250 × 50 mm × 10 mm | ACN | 30 min |
| 88 | Phenomenex luna C18 150 × 40 | water (0.1% TFA)- | 15%-45%, |
| | mm × 15 mm | ACN | 11 min |
| 89 | Phenomenex luna C18 250 × 50 | water (0.1% TFA)- | 26%-46%, |
| | mm × 10 mm | ACN | 10 min |
| 90 | Phenomenex luna C18 250 × 50 | water (0.225% FA)- | 23%-53%, |
| | mm × 10 mm | ACN | 10 min |
| 91 | Phenomenex Gemini-NX C18 | water (0.1% TFA)- | 52%-62%, 7 |
| | 75 × 30 mm × 3 mm | ACN | min |
| 92 | Phenomenex Synergi C18 150 × | water (0.225% FA)- | 14%-38%, 8 |
| | 25 mm × 10 mm | ACN | min |
| 93 | Phenomenex Synergi C18 150 × | water (0.05% HCl)- | 30%-50%, |
| | 25 mm × 10 mm | ACN | 10 min |
| 94 | Phenomenex Gemini-NX C18 | water (0.1% TFA)- | 22%-32%, 7 |
| | 75 × 30 mm × 3 mm | ACN | min |
| 95 | Phenomenex luna C18 150 × 25 | water (0.1% TFA)- | 42%-62%, |
| | mm × 10 mm | ACN | 10 min |
| 96 | Phenomenex Synergi C18 150 × | water (0.225% FA)- | 60%-90%, |
| | 25 mm × 10 mm | ACN | 10 min |
| 97 | Phenomenex Synergi C18 150 × | water (0.225% FA)- | 53%-83%, |
| | 25 mm × 10 mm | ACN | 10 min |
| 98 | Phenomenex Synergi C18 150 × | water (0.225% FA)- | 27%-57%, 9 |
| | 25 mm × 10 mm | ACN | min |
| 99 | Phenomenex luna C18 75 × 30 | water (0.225% FA)- | 60%-80%, 7 |
| | mm × 10 mm | ACN | min |
| 100 | Phenomenex Synergi C18 150 × | water (0.05% HCl)- | 15%-38%, 7 |
| | 25 mm × 10 mm | ACN | min |
| 101 | Boston pH-lex 150 × 25 | water (0.1% TFA)- | 21%-48%, 9 |
| | mm × 10 mm | ACN | mir |
| 102 | Phenomenex Synergi C18 150 × | water (0.05% HCl)- | 10%-30%, 9 |
| | 25 mm × 10 mm | ACN | min |
| 103 | Phenomenex Synergi C18 150 × | water (0.05% HCl)- | 20%-40%, 9 |
| | 25 mm × 10 mm | ACN | min |
| 104 | Phenomenex luna C18 250 × 50 | water (0.1% TFA)- | 15%-40%, |
| | mm × 10 mm | ACN | 10 min |
| 105 | Phenomenex luna C18 150 × 25 | water (0.1% TFA)- | 1%-30%, 10 |
| | mm × 10 mm | ACN | min |
| 106 | Phenomenex luna C18 150 × 25 | water (0.1% TFA)- | 2%-22%, 10 |
| | mm × 10 mm | ACN | min |
| 107 | Phenomenex Synergi C18 150 × | water (0.1% TFA)- | 8%-38%, |
| | 25 mm × 10 mm | ACN | 10 min |
| 108 | Phenomenex Synergi C18 150 × | water (0.1% TFA)- | 12%-38%, |
| | 25 mm × 10 mm | ACN | 10 min |
| 109 | Phenomenex luna C18 150 × 25 | water (0.075% TFA)- | 5%-35%, |
| | mm × 10 mm | ACN | 9 min |
| 110 | Phenomenex Synergi C18 150 × | water (0.1% TFA)- | 3%-33%, |
| | 25 mm × 10 mm | ACN | 10 min |
| 111 | Phenomenex luna C18 150 × 25 | water (0.075% TFA)- | 5%-35%, |
| | mm × 10 mm | ACN | 2 min |
| 112 | Phenomenex luna C18 150 × 25 | water (0.075% TFA)- | 10%-40%, |
| | mm × 10 mm | ACN | 2 min |
| 113 | Phenomenex Synergi C18 150 × | water (0.1% TFA)- | 10%-40%, |
| | 25 mm × 10 mm | ACN | 10 min |
| 114 | Phenomenex luna C18 150 × 25 | water (0.075% TFA)- | 8%-38%, |
| | mm × 10 mm | ACN | 10 min |
| 115 | Phenomenex Synergi C18 150 × | water (0.1% TFA)- | 5%-35%, |
| | 25 mm × 10 mm | ACN | 9 min |
| 116 | Phenomenex luna C18 150 × 25 | water (0.075% TFA)- | 10%-40%, |
| | mm × 10 mm | ACN | 9 min |
| 117 | Phenomenex Synergi C18 150 × | water (0.1% TFA)- | 15%-35%, |
| | 25 mm × 10 mm | ACN | 10 min |
| 118 | Phenomenex luna C18 150 × 25 | water (0.075% TFA)- | 2%-32%, |
| | mm × 10 mm | ACN | 9 min |
| 119 | Phenomenex luna C18 150 × 25 | water (0.1% TFA)- | 7%-37%, |
| | mm × 10 mm | ACN | 10 min |
| 120 | Shim-pack C18 150 × 25 mm × | water (0.225% FA)- | 2%-32%, |
| | 10 mm | ACN | 10 min |
| 121 | Phenomenex Synergi C18 150 × | water (0.225% FA)- | 12%-42%, |
| | 25 mm × 10 mm | ACN | 9 min |
| 122 | Phenomenex luna C18 150 × 25 | water (0.1% TFA)- | 10%-40%, |
| | mm × 10 mm | ACN | 10 min |
| 123 | Phenomenex Gemini-NX C18 | water (0.1% TFA)- | 18%-28%, 7 |
| | 75 × 30 mm × 3 mm | ACN | min |
| 124 | Phenomenex Synergi C18 150 × | water (0.225% FA)- | 10%-40%, |
| | 25 mm × 10 mm | ACN | 10 min |
| 125 | Phenomenex luna C18 150 × 25 | water (0.1% TFA)- | 15%-45%, 9 |
| | mm × 10 mm | ACN | min |
| 126 | Phenomenex luna C18 150 × 25 | water (0.075% TFA)- | 12%-42%, 9 |
| | mm × 10 mm | ACN | min |
| 127 | Phenomenex Synergi C18 150 × | water (0.1% TFA)- | 18%-38%, |
| | 25 mm × 10 mm | ACN | 10 min |
| 128 | Phenomenex luna C18 150 × 25 | water (0.075% TFA)- | 10%-40%, |
| | mm × 10 mm | ACN | 12 min |
| 129 | Phenomenex Synergi C18 150 × | water (0.1% TFA)- | 22%-42%, |
| | 25 mm × 10 mm | ACN | 10 min |
| 130 | Phenomenex Synergi C18 150 × | water (0.1% TFA)- | 20%-40%, |
| | 25 mm × 10 mm | ACN | 10 min |
| 131 | Phenomenex Gemini-NX C18 | water (0.1% TFA)- | 10%-40%, |
| | 75 × 30 mm × 3 mm | ACN | 10 min |
| 132 | Phenomenex luna C18 150 × 25 | water (0.1% TFA)- | 17%-47%, |
| | mm × 10 mm | ACN | 10 min |
| 133 | Phenomenex Gemini-NX C18 | water (0.1% TFA)- | 25%-35%, |
| | 75 × 30 mm × 3 mm | ACN | 10 min |
| 134 | Phenomenex luna C18 150 × 25 | water (0.1% TFA)- | 22%-42%, 9 |
| | mm × 10 mm | ACN | min |
| 135 | Xtimate C18 150 × 40 | water (0.1% | 5%-35%, 10 |
| | mm × 10 mm | ammonium | min |
| | | hydroxide)-ACN | |
| 136 | Phenomenex Gemini-NX C18 | water (0.1% TFA)- | 25%-35%, 7 |
| | 75 × 30 mm × 3 mm | ACN | min |
| 137 | Phenomenex Gemini-NX C18 | water (0.1% TFA)- | 20%-30%, 7 |
| | 75 × 30 mm × 3 mm | ACN | min |
| 138 | Phenomenex Synergi C18 150 × | water (0.05% HCl)- | 12%-32%, 9 |
| | 25 mm × 10 mm | ACN | min |
| 139 | Phenomenex luna C18 150 × 25 | water (0.05% HCl)- | 1%-30%, 9 |
| | mm × 10 mm | ACN | min |
| 140 | Phenomenex Synergi C18 150 × | water (0.05% HCl)- | 5%-35%, 10 |
| | 25 mm × 10 mm | ACN | min |
| 141 | Shim-pack C18 150 × 25 mm × | water (0.225% FA)- | 3%-27%, 8 |
| | 10 mm | ACN | min |
| 142 | Phenomenex Synergi C18 150 × | water (0.05% HCl)- | 11%-33%, |
| | 25 mm × 10 mm | ACN | 11 min |
| 143 | Phenomenex Gemini-NX C18 | water (0.1% | 3%-33%, 7 |
| | 75 × 30 mm × 3 mm | ammonium | min |
| | | hydroxide)-ACN | |
| 144 | Phenomenex Synergi C18 150 × | water (0.1% TFA)- | 21%-51%, |
| | 25 mm × 10 mm | ACN | 10 min |
| 145 | Phenomenex Synergi C18 150 × | water (0.1% TFA)- | 25%-45%, |
| | 25 mm × 10 mm | ACN | 10 min |
| 146 | Phenomenex luna C18 150 × 25 | water (0.1% TFA)- | 24%-44%, |
| | mm × 10 mm | ACN | 10 min |
| 147 | Phenomenex luna C18 150 × 25 | water (0.1% TFA)- | 11%-41%, |
| | mm × 10 mm | ACN | 10 min |
| 148 | Xtimate C18 150 × 40 | water (0.1% | 27%-57%, |
| | mm × 10 mm | ammonium | 10 min |
| | | hydroxide)-ACN | |
| 149 | Phenomenex luna C18 200 × 40 | water (0.1% TFA)- | 32%-52%, |
| | mm × 10 mm | ACN | 10 min |
| 150 | Phenomenex luna C18 200 × 40 | water (0.1% TFA)- | 18%-38%, |
| | mm × 10 mm | ACN | 10 min |
| 151 | Unisil 3-100 C18 Ultra150 × 50 | water (0.225% FA)- | 8%-38%, 10 |
| | mm × 3 mm | ACN | min |
| 152 | Unisil 3-100 C18 Ultra150 × 50 | water (0.225% FA)- | 20%-40%, |
| | mm × 3 mm | ACN | 10 min |
| 153 | Phenomenex Synergi C18 150 × | water (0.225% FA)- | 3%-33%, 10 |
| | 25 mm × 10 mm | ACN | min |
| 154 | Phenomenex luna C18 200 × 40 | water (0.225% FA)- | 20%-50%, |
| | mm × 10 mm | ACN | 10 min |
| 155 | Phenomenex luna C18 150 × 25 | water (0.075% TFA)- | 35%-50% 9 |
| | mm × 10 mm | ACN | min |
| 156 | Phenomenex luna C18 150 × 25 | water (0.1% TFA)- | 15%-45%, |
| | mm × 10 mm | ACN | 10 min |
| 157 | Phenomenex luna C18 150 × 25 | water (0.1% TFA)- | 8%-38%, 10 |
| | mm × 10 mm | ACN | min |
| 158 | Phenomenex luna C18 150 × 25 | water (0.1% TFA)- | 6%-36%, 10 |
| | mm × 10 mm | ACN | min |
| 159 | Phenomenex luna C18 150 × 25 | water (0.1% TFA)- | 9%-39%, 10 |
| | mm × 10 mm | ACN | min |
| 160 | Phenomenex Synergi C18 150 × | water (0.1% TFA)- | 12%-32%, |
| | 25 mm × 10 mm | ACN | 10 min |
| 161 | Phenomenex Synergi C18 150 × | water (0.1% TFA)- | 20%-30%, |
| | 25 mm × 10 mm | ACN | 10 min |
| 162 | Phenomenex luna C18 150 × 25 | water (0.1% TFA)- | 12%-32%, |
| | mm × 10 mm | ACN | 10 min |
| 163 | Phenomenex luna C18 150 × 25 | water (0.075% TFA)- | 8%-38%, 9 |
| | mm × 10 mm | ACN | min |
| 164 | Phenomenex luna C18 150 × 25 | water (0.1% TFA)- | 2%-31%, 10 |
| | mm × 10 mm | ACN | min |
| 165 | Phenomenex luna C18 150 × 25 | water (0.1% TFA)- | 2%-25%, 10 |
| | mm × 10 mm | ACN | min |
| 166 | Phenomenex Synergi C18 150 × | water (0.1% TFA)- | 2%-32%, 10 |
| | 25 mm × 10 mm | ACN | min |
| 167 | Phenomenex luna C18 150 × 25 | water (0.075% TFA)- | 1%-30%, 9 |
| | mm × 10 mm | ACN | min |
| 168 | Phenomenex Synergi C18 150 × | water (0.1% TFA)- | 6%-26%, 10 |
| | 25 mm × 10 mm | ACN | min |
| 169 | Phenomenex luna C18 150 × 25 | water (0.075% TFA)- | 5%-30%, 10 |
| | mm × 10 mm | ACN | min |
| 170 | Phenomenex Synergi C18 150 × | water (0.1% TFA)- | 10%-30%, |
| | 25 mm × 10 mm | ACN | 10 min |
| 171 | Waters Xbridge 150 × 25 mm × | water (0.05% | 8%-38%, 10 |
| | 5 mm | ammonium | min |
| | | hydroxide)-ACN | |
| 172 | Phenomenex luna C18 150 × 25 | water (0.1% TFA)- | 16%-46%, |
| | mm × 10 mm | ACN | 10 min |
| 173 | Phenomenex luna C18 150 × 25 | water (0.1% TFA)- | 14%-44%, |
| | mm × 10 mm | ACN | 10 min |
| 174 | Xtimate C18 150 × 40 mm × 10 | water (0.05% | 1%-31%, 10 |
| | mm | ammonium | min |
| | | hydroxide)-ACN | |
| 175 | Waters Xbridge 150 × 25 mm × | water (0.05% | 34%-54%, |
| | 5 mm | ammonium | 10 min |
| | | hydroxide)-ACN | |
| 176 | Phenomenex luna C18 150 × 25 | water (0.1% TFA)- | 12%-42%, |
| | mm × 10 mm | ACN | 10min |
| 177 | Waters Xbridge 150 × 25 mm × | water (0.05% | 9%-37%, 10 |
| | 5 mm | ammonium | min |
| | | hydroxide)-ACN | |
| 178 | Waters Xbridge 150 × 25 mm × | water (0.05% | 4%-34%, 10 |
| | 5 mm | ammonium | min |
| | | hydroxide)-ACN | |
| 179 | Unisil 3-100 C18 Ultra150 × 50 | water (0.225% FA)- | 20%-50%, |
| | mm × 3 mm | ACN | 10 min |
| 180 | Waters Xbridge 150 × 25 | water (0.05% | 15%-45%, |
| | mm × 5 mm | ammonium | 10 min |
| | | hydroxide)-ACN | |
| 181 | Waters Xbridge 150 × 25 | water (0.05% | 28%-58%, |
| | mm × 5 mm | ammonium | 10 min |
| | | hydroxide)-ACN | |
| 182 | Waters Xbridge 150 × 25 | water (0.05% | 9%-36%, 10 |
| | mm × 5 mm | ammonium | min |
| | | hydroxide)-ACN | |
| 183 | Phenomenex luna C18 150 × 25 | water (0.1% TFA)- | 25%-45%, |
| | mm × 10 mm | ACN | 10 min |
| 184 | Phenomenex Gemini-NX C18 | water (0.1% TFA)- | 35%-45%, 7 |
| | 75 × 30 mm × 3 mm | ACN | min |
| 185 | Waters Vinridis Silica 2-EP OBD | Hexane-EtOH | 25%-45%, |
| | 150 × 50 mm × 5 mm | | 10 min |
| 186 | Phenomenex luna C18 150 × 25 | water (0.225% FA)- | 6%-36%, 10 |
| | mm × 10 mm | ACN | min |
| 187 | Phenomenex luna C18 150 × 25 | water (0.1% TFA)- | 46%-76%, |
| | mm × 10 mm | ACN | 7min |
| 188 | Waters Xbridge 150 × 25 | water (10 mM | 21%-51%, |
| | mm × 5 mm | NH₄HCO₃)-ACN | 10 min |
| 189 | Waters Xbridge 150 × 25 | water (0.05% | 13%-43%, 9 |
| | mm × 5 mm | ammonium | min |
| | | hydroxide)-ACN | |
| 190 | Phenomenex luna C18 150 × 25 | water (0.1% TFA)- | 30%-60%, 7 |
| | mm × 10 mm | ACN | min |

### ABBREVIATIONS

Wherein the following abbreviations have been used, the following meanings apply:
ACN is acetonitrile,
AM is analytical method,
aq. is aqueous,
Boc₂O is Di-tert-butyl dicarbonate,
Br₂ is Bromine solution,
CBr₄ is carbon tetrabromide,
CDI is 1,1′-carbonyldiimidazole,
CHCl₃-d is deuterated chloroform,
Cs₂CO₃ is cesium carbonate,
CsF is cesium fluoride,
CuI is copper iodide,
DCE is dichloroethane,
DCM is dichloromethane,
DIPEA is N,N-diisopropylethylamine,
DMAP is dimethylaminopyridine,
DME is 1,2-dimethoxyethane,
DMF is N,N-dimethylformamide,
DMP is Dess-Martin periodinane,
DMS is dimethylsulfide,
DMSO is dimethyl sulfoxide,
DMSO-d₆ is dimethyl sulfoxide,
DPPF is 1,1′-ferrocenediyl-bis(diphenylphosphine),
EA is Ethyl acetate,
EDCI is N-(3-dimethylaminopropyl)-N′-ethylcarbodiimide hydrochloride,
EtOH is ethanol,
FA is formic acid,
Fmoc is 9-fluorenylmethoxycarbonyl,
h is Hours,
NMR is nuclear magnetic resonance,
HATU is (1-[Bis(dimethylamino)methylene]-1H-1,2,3-triazolo[4,5-b]pyridinium 3-oxide hexafluorophosphate,
HCl is hydrochloric acid,
HOBt is 1-hydroxybenzotriazole,
H₂O is water,
HPLC is high performance liquid chromatography,
KF is potassium fluoride,
K₂CO₃ is potassium carbonate,
LAH is lithium aluminum hydride,
LCMS is Liquid Chromatography Mass Spectrometry,
LiOH·H₂O is Lithium hydroxide monohydrate,
MeOH is methanol,
MeOH-d₄ is deuterated methanol,
min is minutes,
MS are molecular sieves,
MTBE is methyltertbutylether,
N₂ is nitrogen gas,
NaBH(OAc)₃ is sodium triacetoxyborohydride,
NaBH₃CN is sodium cyanoborohydride
NaH is sodium hydride,
NaHCO₃ is sodium bicarbonate,
NaOAc is sodium acetate,
NaOH is sodium hydroxide,
NaOMe is sodium methoxide,
NH₄Cl is ammonium chloride,
Na₂SO₄ is anhydrous sodium sulphate,
n-BuLi is n-Butyllithium,
NCS is N-chlorosuccinimide,
Pd/C is palladium on carbon,
Pd(PPh₃)₄ is Tetrakis(triphenylphosphine)palladium(0),
Pd(dppf)Cl₂ is [1,1′-Bis(diphenylphosphino)ferrocene]dichloropalladium(II),
Pd(dppf)Cl₂·CHCl₂ is [1,1′-Bis(diphenylphosphino)ferrocene]dichloropalladium(II), complex with dichloromethane,
PE is Petroleum ether,
PM is purification method,
POCl₃ is phosphorous oxychloride,
PPh₃ is triphenylphosphine,
rt is retention time,
RT is room temperature,
RuPhos Pd G3 is (2-Dicyclohexylphosphino-2′,6′-diisopropoxy-1,1′-biphenyl)[2-(2′-amino-1,1′-biphenyl)]palladium(II) methanesulfonate,
SEM is silylethoxymethyl,
SOCl₂ is thionyl chloride,
TBAC is tetrabutylammonium chloride,
TBAF is tetrabutylammonium fluoride,
TBAI is tetramethylammonium iodide,
TEA is triethylamine,
TFA is trifluoroacetic acid,
TFAA is trifluoroacetic anhydride,
THF is tetrahydrofuran,
TLC is thin layer chromatography,
TMEDA is N′-tetramethylethylenediamine,
T3P is propylphosphonic anhydride
XPhos-Pd-G3 is (2-Dicyclohexylphosphino-2′,4′,6′-triisopropyl-1,1′-biphenyl)[2-(2′-amino-1,1′-biphenyl)]palladium(II) methanesulfonate and
9-BBN is 9-Borabicyclo[3.3.1]nonane.

### Preparation of Intermediates

The following Preparations describe the methods used for common intermediates required for synthesis of the Examples.

Compound 1.1 may be prepared according to the method described in J. Med. Chem. 2011, 54 (2), 635-654.

### Synthesis of Intermediate A

### Methyl 5-((2-((tert-butoxycarbonyl)amino)ethyl)amino)benzo[c][2,6]naphthyridine-8-carboxylate, 1.3

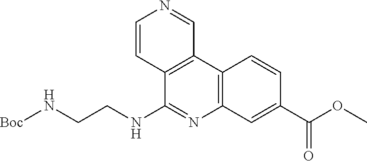 [SMILES: COC(=O)c1ccc2c(c1)nc(NCCNC(=O)OC(C)(C)C)c1ccncc12]

To a stirred mixture of compound 1.1 (Journal of Medicinal Chemistry, 2011, 54 (2), 635-654) (6 g, 20.68 mmol) and tert-butyl (2-aminoethyl)carbamate (4.55 mL, 28.96 mmol) in DMSO (60 mL) was added DIPEA (9 mL, 51.67 mmol) at RT, the mixture was heated to 70° C. and stirred for 17 h. The reaction mixture was added to H₂O (300 mL), precipitating a grey solid that was collected by filtration and dried under vacuum. This crude product was triturated in EtOH (60 mL), filtered and dried under vacuum to afford compound 1.3 (7.2 g, 87.8% yield) as a grey solid.

¹H NMR (400 MHz, DMSO-d₆) δ: 10.07 (s, 1H), 8.90 (d, J=5.6 Hz, 1H), 8.76 (d, J=8.4 Hz, 1H), 8.23 (d, J=5.6 Hz, 1H), 8.17 (s, 1H), 8.05 (t, J=4.8 Hz, 1H), 7.83 (dd, J=8.4 Hz, 1.6 Hz, 1H), 7.03 (t, J=5.6 Hz, 1H), 3.92 (s, 3H), 3.65 (q, J=5.6 Hz, 2H), 3.33-3.29 (m, 2H), 1.36 (s, 9H) ppm.

### Methyl 5-((2-aminoethyl)amino)benzo[c][2,6]naphthyridine-8-carboxylate, Intermediate A

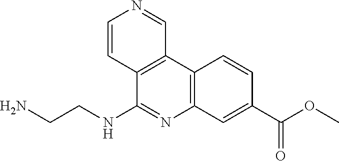 [SMILES: COC(=O)c1ccc2c(c1)nc(NCCN)c1ccncc12]

A mixture of compound 1.3 (1.11 g, 2.88 mmol) in a solution of HCl in 1,4-dioxane (4 M, 10 mL, 40 mmol) was stirred at 25° C. for 5 h. The reaction mixture was concentrated in vacuo to afford Intermediate A (1.33 g, HCl salt) as a yellow solid, which was used directly without further purification.

LCMS (AM1): rt=0.561 min, (297.0 [M+H]⁺), 88.7% purity.

### Synthesis of Intermediate 1.11

### 4-(((2-chloro-[1,1′-biphenyl]-4-yl)methyl)amino)butan-1-ol 1.5

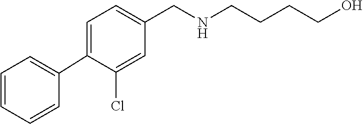 [SMILES: OCCCCNCc1ccc(-c2ccccc2)c(Cl)c1]

A mixture of 3-chloro-4-phenylbenzaldehyde (CAS 57592-44-6, 10 g, 42.00 mmol) (Bioorganic and Medicinal Chemistry, 2017, 25 (13), 3471-3482), 4-aminobutan-1-ol (8.01 g, 89.88 mmol) and 4 Å MS (20 g) in MeOH (200 mL) was stirred at RT for 18 h, and then NaBH₃CN (9 g, 143.22 mmol) added. The mixture was stirred for 4 h at RT. The reaction mixture was filtered, the filtrate was concentrated in vacuo to give a residue that was added to H₂O (200 mL) and the aq. mixture was extracted with EA (200 ml×3). The combined organic phases were washed with brine (300 mL), dried (Na₂SO₄), filtered and concentrated in vacuo. The crude product was purified (PM16) to give compound 1.5 (6.9 g, 23.77 mmol, 51.6% yield) as a light brown gum.

¹H NMR (CHCl₃-d, 400 MHz) δ: 7.37-7.34 (m, 6H), 7.25-7.19 (m, 2H), 3.74 (s, 2H), 3.56 (m, 2H), 3.38 (bs, 2H), 2.68 (t, J=5.6 Hz, 2H), 1.63-1.58 (m, 4H) ppm.

### tert-butyl ((2-chloro-[1,1′-biphenyl]-4-yl)methyl)(4-hydroxybutyl)carbamate 1.6

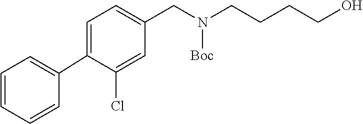 [SMILES: CC(C)(C)OC(=O)N(CCCCO)Cc1ccc(-c2ccccc2)c(Cl)c1]

To a mixture of compound 1.5 (6.9 g, 23.77 mmol) in THF (45 mL) and H₂O (15 mL) was added NaHCO₃ (4.00 g, 47.62 mmol) and (Boc)₂O (6 g, 27.49 mmol) at RT. The mixture was stirred for 18 h. To the reaction mixture was added H₂O (100 mL), extracted with EA (100 mL×3) and the combined organic phases washed (brine, 100 mL), dried (Na₂SO₄), filtered and concentrated in vacuo. The residue was purified (PM17) to afford compound 1.6 (7.0 g, 17.99 mmol, 75% yield) as a light brown oil.

LCMS (AM1): rt=0.839 min, (334.1 [M−^{t}Bu+2H]⁺), 99.3% purity.

### tert-butyl ((2-chloro-[1,1′-biphenyl]-4-yl)methyl)(4-oxobutyl)carbamate 1.7

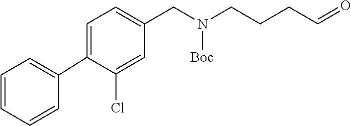 [SMILES: CC(C)(C)OC(=O)N(CCCC=O)Cc1ccc(-c2ccccc2)c(Cl)c1]

To a solution of compound 1.6 (1.5 g, 3.85 mmol) in DCM (30 mL) was added (1,1,1-trisacetyloxy)-1,1-dihydro-1,2-benziodoxol-3(1H)-one (DMP, 2 g, 4.72 mmol) at RT. The reaction mixture was then stirred for 15 h. The reaction mixture filtered, the filtrate concentrated in vacuo and purified (PM18) to afford compound 1.7 (1.01 g, 62.9% yield) as a colourless oil.

LCMS (AM1): rt=0.979 min, (332.0 [M−^{t}Bu+2H]⁺), 81.6% purity.

### methyl 5-((2-((4-((tert-butoxycarbonyl)((2-chloro-[1,1′-biphenyl]-4-yl)methyl)amino)butyl)amino)ethyl)amino)benzo[c][2,6]naphthyridine-8-carboxylate 1.8

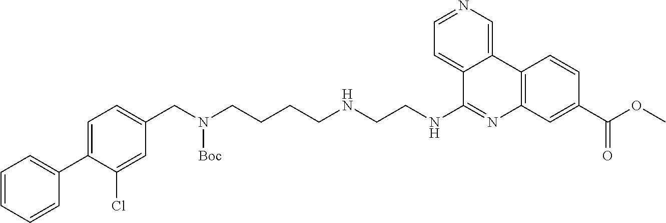 [SMILES: COC(=O)c1ccc2c(c1)nc(NCCNCCCCN(Cc1ccc(-c3ccccc3)c(Cl)c1)C(=O)OC(C)(C)C)c1ccncc12]

To a mixture of compound 1.7 (1 g, 2.40 mmol) and NaOAc (1.06 g, 12.92 mmol) in MeOH (20 mL) was added Intermediate A (2.23 g, 3.35 mmol, HCl salt) at RT and the reaction mixture was stirred for 1 hr. NaBH₃CN (324 mg, 5.16 mmol) was added and the reaction mixture stirred at RT for 17 h. The reaction mixture was filtered and the filter cake was washed with EA (20 mL×2). The filtrate was then concentrated in vacuo and purified (PM22) to afford compound 1.8 (240 mg, 12.4% yield, TFA salt) as a yellow solid.

LCMS (AM2): rt=0.879 min, (668.3 [M+H]⁺), 96.9% purity.

### methyl 5-((2-((tert-butoxycarbonyl)(4-((tert-butoxycarbonyl)((2-chloro-[1,1′-biphenyl]-4-yl)methyl)amino)butyl)amino)ethyl)amino)benzo[c][2,6]naphthyridine-8-carboxylate 1.9

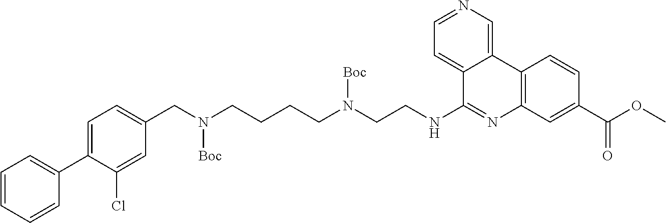 [SMILES: COC(=O)c1ccc2c(c1)nc(NCCN(CCCCN(Cc1ccc(-c3ccccc3)c(Cl)c1)C(=O)OC(C)(C)C)C(=O)OC(C)(C)C)c1ccncc12]

To a mixture of compound 1.8 (240 mg, 0.298 mmol, TFA salt) in THF (10 mL) was added DMAP (20 mg, 0.163 mmol), Boc₂O (142.5 mg, 0.653 mmol) and TEA (1.80 mmol, 0.25 mL), sequentially at RT. The reaction mixture was then heated to reflux for 18 h. The reaction mixture was concentrated in vacuo to afford compound 1.9 (330 mg) as a brown solid, which was used directly without further purification.

LCMS (AM1): rt=0.946 min, (768.3 [M+H]⁺), 91.6% purity.

### 5-((2-((tert-butoxycarbonyl)(4-((tert-butoxycarbonyl)((2-chloro-[1,1′-biphenyl]-4-yl)methyl)amino)butyl)amino)ethyl)amino)benzo[c][2,6]naphthyridine-8-carboxylic acid Intermediate B

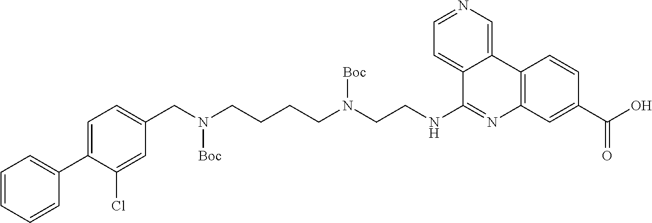 [SMILES: CC(C)(C)OC(=O)N(CCCCN(Cc1ccc(-c2ccccc2)c(Cl)c1)C(=O)OC(C)(C)C)CCNc1nc2cc(C(=O)O)ccc2c2cnccc12]

A mixture of compound 1.9 (330 mg, 0.43 mmol) and LiOH. H₂O (180 mg, 4.29 mmol) in H₂O (8 mL) and THF (16 mL) was stirred at 25° C. for 8 h. The reaction mixture was added to aq. HCl (0.1 N, 50 mL) and the resulting aq. mixture was extracted with EA (20 mL×3). The organic phases combined, washed (brine, 30 mL), dried (Na₂SO₄), filtered and concentrated in vacuo to afford Intermediate B (200 mg) as a yellow solid, which was used directly without further purification.

LCMS (AM1): rt=0.890 min, (754.3 [M+H]⁺), 89% purity.

### tert-butyl (4-((tert-butoxycarbonyl)(2-((8-carbamoylbenzo[c][2,6]naphthyridin-5-yl)amino)ethyl)amino)butyl)((2-chloro-[1,1′-biphenyl]-4-yl)methyl)carbamate 1.10

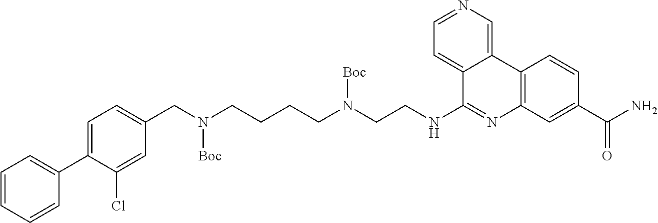 [SMILES: CC(C)(C)OC(=O)N(CCCCN(Cc1ccc(-c2ccccc2)c(Cl)c1)C(=O)OC(C)(C)C)CCNc1nc2cc(C(N)=O)ccc2c2cnccc12]

To a mixture of Intermediate B (200 mg, 0.27 mmol) in DMF (10 mL) was added NH₄Cl (400 mg, 7.48 mmol), HOBt (160 mg, 1.18 mmol,), DIPEA (1 mL) and EDCI (230 mg, 1.20 mmol), sequentially at RT. The reaction mixture was then heated to 70° C. for 8 h. The reaction mixture was poured into H₂O (100 mL) and extracted with EA (30 mL×2). The combined organic phases were washed (brine, 30 mL), dried (Na₂SO₄), filtered and concentrated in vacuo to afford compound 1.10 (200 mg) as a yellow gum, which was used directly without further purification.

LCMS (AM1): rt=0.877 min, (753.3 [M+H]⁺), 88.0% purity.

### tert-butyl (4-((tert-butoxycarbonyl)(2-((8-cyanobenzo[c][2,6]naphthyridin-5-yl)amino)ethyl)amino)butyl)((2-chloro-[1,1′-biphenyl]-4-yl)methyl)carbamate 1.11

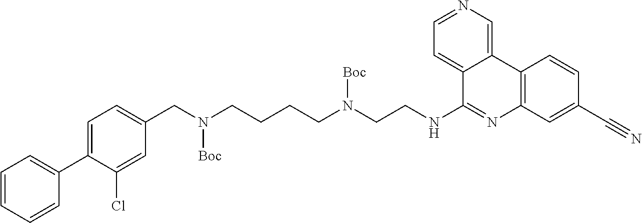 [SMILES: CC(C)(C)OC(=O)N(CCCCN(Cc1ccc(-c2ccccc2)c(Cl)c1)C(=O)OC(C)(C)C)CCNc1nc2cc(C#N)ccc2c2cnccc12]

To a solution of compound 1.10 (195 mg, 0.26 mmol) and TEA (1 mL) in DCM (20 mL) was added TFAA (0.5 mL) dropwise at RT, the reaction mixture was then stirred for 12 h. The reaction mixture was concentrated in vacuo and purified (PM23) to afford compound 1.11 (130 mg, 68% yield) as a light yellow solid.

LCMS (AM1): rt=1.024 min, (735.3 [M+H]⁺), 94.6% purity.

### Synthesis of Intermediate 1.17

### 5-((2-((tert-butoxycarbonyl)amino)ethyl)amino)benzo[c][2,6]naphthyridine-8-carboxylic acid 1.12

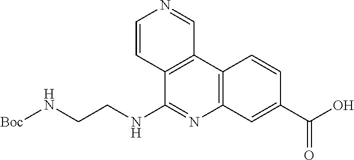 [SMILES: CC(C)(C)OC(=O)NCCNc1nc2cc(C(=O)O)ccc2c2cnccc12]

A mixture of compound 1.3 (7.2 g, 18.16 mmol) and LiOH·H₂O (7.62 g, 181.62 mmol) in THF (100 mL) and H₂O (50 mL) was stirred at RT for 19 h. The reaction mixture was added to H₂O (200 mL) and the pH of the mixture adjusted to pH4 with aq. HCl (1 N). The precipitate was collected by filtration and dried under vacuum to afford compound 1.12 (6.44 g, 91.3% yield) as a light yellow solid.

¹H NMR (400 MHz, DMSO-d₆) δ: 13.14 (br s, 1H), 10.10 (s, 1H), 8.93 (d, J=5.2 Hz, 1H), 8.78 (d, J=8.4 Hz, 1H), 8.32 (br s, 2H), 7.87 (d, J=1.2 Hz, 1H), 7.07 (s, 1H), 3.69 (m, 2H), 3.36-3.34 (m, 2H), 1.35 (s, 9H) ppm.

### tert-butyl (2-((8-carbamoylbenzo[c][2,6]naphthyridin-5-yl)amino)ethyl)carbamate 1.13

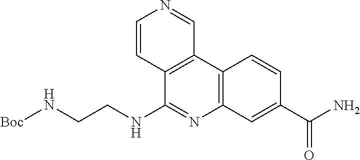 [SMILES: CC(C)(C)OC(=O)NCCNc1nc2cc(C(N)=O)ccc2c2cnccc12]

To a mixture of compound 1.12 (6.44 g, 16.84 mmol), NH₄Cl (9 g, 168.26 mmol), HOBt (4.55 g, 33.68 mmol) and DIPEA (14.6 mL) in DMF (150 mL) was added EDCI (6.46 g, 33.68 mmol) at RT. The reaction mixture was heated to 70° C. and stirred for 3 h. The reaction mixture was added to H₂O (600 mL) and a white solid precipitated which was collected by filtration. The filter cake was triturated in EA:MeOH (400 mL, v:v=20:1). The precipitate was collected by filtration and dried under vacuum to afford compound 1.13 (6.39 g, 99.5% yield) as a white solid.

¹H NMR (400 MHz, DMSO-d₆) δ: 10.07 (s, 1H), 8.87 (d, J=5.2 Hz, 1H), 8.70 (d, J=8.4 Hz, 1H), 8.22 (d, J=6.0 Hz, 1H), 8.15 (s, 1H), 8.14 (d, J=1.6 Hz, 1H), 7.95-7.91 (m, 1H), 7.81 (dd, J=8.4 Hz, 1.6 Hz, 1H), 7.42 (s, 1H), 7.03 (t, J=5.2 Hz, 1H), 3.67-3.63 (m, 2H), 3.35-3.33 (m, 2H), 1.37 (s, 9H) ppm.

### tert-butyl (2-((8-aminobenzo[c][2,6]naphthyridin-5-yl)amino)ethyl)carbamate 1.14

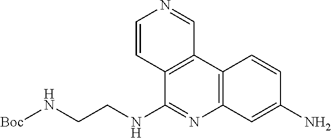 [SMILES: CC(C)(C)OC(=O)NCCNc1nc2cc(N)ccc2c2cnccc12]

To a solution of NaOH (590 mg, 14.76 mmol) in H₂O (20 mL) was added Br₂ (0.24 mL, 4.66 mmol) at 0° C. The reaction was stirred at 0° C. for 30 min, then compound 1.13 (700 mg, 1.84 mmol) added. The reaction mixture was stirred at 0° C. for 30 min, then the mixture was heated to 70° C. and stirred for 2 h. The pH of the combined reaction mixture was adjusted to pH7 with aq. HCl (1 N). The resultant mixture was extracted with EA:MeOH (v/v=20:1, 100 mL×3). The combined organic phase was washed (brine, 200 mL), dried (Na₂SO₄), filtered and concentrated in vacuo to afford compound 1.14 (720 mg) as a brown solid, which was used directly without further purification.

LCMS (AM2): rt=0.634 min, (354.1 [M+H]⁺), 90.1% purity.

### tert-butyl (2-((8-acetamidobenzo[c][2,6]naphthyridin-5-yl)amino)ethyl)carbamate 1.15

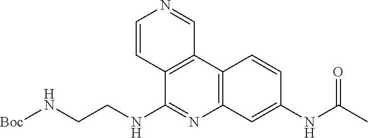 [SMILES: CC(=O)Nc1ccc2c(c1)nc(NCCNC(=O)OC(C)(C)C)c1ccncc12]

To a mixture of compound 1.14 (80 mg, 0.226 mmol), TEA (0.1 mL, 0.72 mmol) and N, N-dimethylpyridin-4-amine (14 mg, 0.115 mmol) in DCM (2 mL) was added acetic anhydride (0.1 mL, 0.27 mmol) at RT. The reaction mixture was heated to 50° C. and stirred for 2 h. The reaction mixture was added to H₂O (40 mL), extracted with EA (20 mL×2) and the combined organic phase was washed (brine, 20 mL), dried (Na₂SO₄), filtered and concentrated in vacuo to afford compound 1.15 (90 mg) as a brown solid, which was used directly without further purification.

LCMS (AM2): rt=0.605 min, (396.2 [M+H]⁺), 88.2% purity.

### N-(5-((2-aminoethyl)amino)benzo[c][2,6]naphthyridin-8-yl)acetamide 1.16

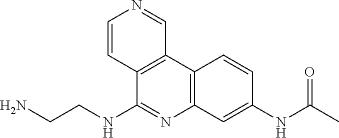 [SMILES: CC(=O)Nc1ccc2c(c1)nc(NCCN)c1ccncc12]

To a mixture of compound 1.15 (90 mg, 0.23 mmol) in DCM (2 mL) was added TFA (0.5 mL, 6.75 mmol) at RT, and the mixture was stirred for 2 h. The reaction mixture was concentrated in vacuo to afford compound 1.16 (100 mg, TFA salt) as a brown oil, which was used directly without further purification.

LCMS (AM4): rt=0.196 min, (296.1 [M+H]⁺), 89.6% purity.

### tert-butyl (4-((2-((8-acetamidobenzo[c][2,6]naphthyridin-5-yl)amino)ethyl)amino)butyl)((2-chloro-[1,1′-biphenyl]-4-yl)methyl)carbamate 1.17

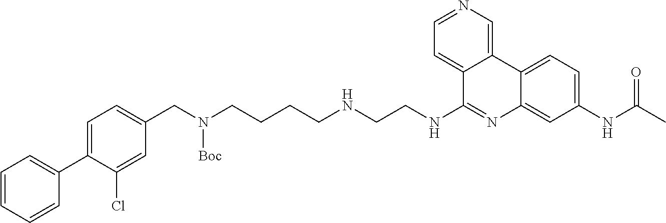 [SMILES: CC(=O)Nc1ccc2c(c1)nc(NCCNCCCCN(Cc1ccc(-c3ccccc3)c(Cl)c1)C(=O)OC(C)(C)C)c1ccncc12]

To a mixture of compound 1.7 (90 mg, 0.193 mmol) and compound 1.16 (100 mg, 0.244 mmol, TFA salt) in DCE (4 mL) was added NaOAc (76 mg, 0.926 mmol) at RT. The mixture was stirred for 2 h, NaBH(OAc)₃ (100 mg, 0.472 mmol) was added in one portion and the reaction mixture stirred for 15 h. The reaction mixture was concentrated in vacuo and the crude product purified (PM24) to afford compound 1.17 (37 mg, TFA salt) as a yellow solid.

LCMS (AM4): rt=0.875 min, (667.3 [M+H]⁺), 71% purity.

### Synthesis of Intermediate 1.24

### tert-butyl (4-(cyanomethoxy)butyl)carbamate, 1.19

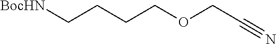 [SMILES: CC(C)(C)OC(=O)NCCCCOCC#N]

To a mixture of tert-butyl (4-hydroxybutyl)carbamate (7 g, 36.99 mmol) and 2-bromoacetonitrile (8.87 g, 73.98 mmol) in DCM (100 mL) was added silver (I) oxide (18.55 g, 80.05 mmol) and TBAI (2.94 g, 7.96 mmol) at 25° C., the mixture was stirred for 16 h. The mixture was filtered, the filtrate washed with aq. NaHCO₃ (100 mL), dried with Na₂SO₄, filtered and concentrated in vacuo. The crude product was purified (PM8) to afford compound 1.19 (1.0 g, 4.38 mmol, 11.8% yield) as a yellow oil.

¹H NMR (CHCl₃-d, 400 MHz) δ: 4.56 (br s, 1H), 4.24 (s, 2H), 3.62-3.59 (t, 2H), 3.18-3.13 (m, 2H), 1.71-1.63 (m, 2H), 1.58-1.53 (m, 2H), 1.45 (s, 9H) ppm.

### tert-butyl (4-(2-aminoethoxy)butyl)carbamate, 1.20

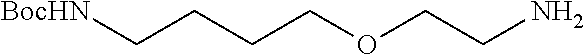 [SMILES: CC(C)(C)OC(=O)NCCCCOCCN]

To a solution of compound 1.19 (1.0 g, 4.38 mmol) in MeOH (10 mL) was added ammonium hydroxide (2 mL, 25% wt.) and Raney nickel (100 mg, 1.17 mmol) under N₂ at 25° C. The suspension was degassed and purged with H₂ (×3). The mixture was stirred under H₂ (45 psi) at 25° C. for 16 h. The mixture was filtered and the filtrate concentrated in vacuo to afford compound 1.20 (1 g) as a green oil, which was used directly.

### tert-butyl (4-(2-(((2-chloro-[1,1′-biphenyl]-4-yl)methyl)amino)ethoxy)butyl)carbamate 1.21

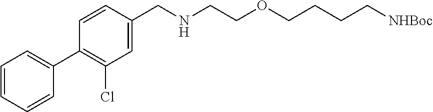 [SMILES: CC(C)(C)OC(=O)NCCCCOCCNCc1ccc(-c2ccccc2)c(Cl)c1]

A mixture of 3-chloro-4-phenylbenzaldehyde (CAS 57592-44-6, 480 mg, 2.22 mmol) and compound 1.20 (640 mg, 2.75 mmol) in MeOH (10 mL) was stirred at 25° C. for 6 h. NaBH(OAc)₃ (2.82 g, 13.29 mmol) was added and the mixture stirred for 12 h. The reaction mixture was concentrated in vacuo to give a residue, which was purified (PM25) to afford compound 1.21 (730 mg, 1.69 mmol, 76.1% yield, FA salt, 100% purity) as a white solid.

LCMS (AM3): rt=0.865 min, (433.5 [M+H]⁺), 96.77% purity.

### (9H-fluoren-9-yl)methyl (2-(4-((tert-butoxycarbonyl)amino)butoxy)ethyl)((2-chloro-[1,1′-biphenyl]-4-yl)methyl)carbamate 1.22

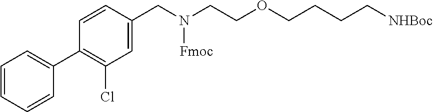

To a mixture of compound 1.21 (600 mg, 1.39 mmol) and NaHCO₃ (233 mg, 2.77 mmol) in dioxane (5 mL) and H₂O (5 mL) was added (9H-fluoren-9-yl)methyl carbonochloridate (396 mg, 1.53 mmol) at 0° C. The mixture was warmed to 20° C. and stirred for 1 h. The mixture was diluted with H₂O (20 mL) and the aq. phase was extracted with EA (20 mL×3). The combined organic phase were washed (brine, 50 mL), dried Na₂SO₄, filtered and concentrated in vacuo. The residue was purified (PM2) to afford compound 1.22 (800 mg, 1.22 mmol, 88.1% yield, 100% purity) as a colourless oil.

LCMS (AM3): rt=1.152 min, (655.4 [M+H]⁺), 100% purity.

### (9H-fluoren-9-yl)methyl(2-(4-aminobutoxy)ethyl)((2-chloro-[1,1′-biphenyl]-4-yl)methyl)carbamate 1.23

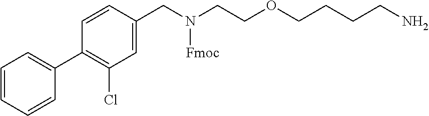

A mixture of compound 1.22 (800 mg, 1.22 mmol) in a solution of HCl in 1,4-dioxane (4 M, 6 mL) was stirred at 20° C. for 1 h. The reaction mixture was concentrated in vacuo to afford compound 1.23 (700 mg) as a colourless oil which was used directly without further purification.

LCMS (AM3): rt=0.886 min, (555.3 [M+H]⁺), 87% purity.

### methyl 5-((4-(2-(((2-chloro-[1,1′-biphenyl]-4-yl)methyl)amino)ethoxy)butyl)amino)benzo[c][2,6]naphthyridine-8-carboxylate 1.24

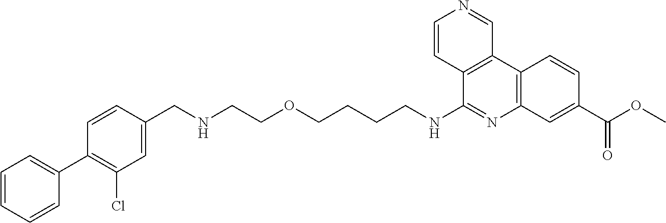 [SMILES: COC(=O)c1ccc2c(c1)nc(NCCCCOCCNCc1ccc(-c3ccccc3)c(Cl)c1)c1ccncc12]

To a mixture of compound 1.23 (650 mg, 1.17 mmol) and compound 1.1 (363 mg, 1.17 mmol) in DMSO (4 mL) was added DIPEA (482 mg, 3.73 mmol) at RT. The mixture was heated to 70° C. and stirred for 12 h. The reaction mixture was filtered, the filtrate concentrated in vacuo and purified (PM26) to afford compound 1.24 (350 mg, 0.603 mmol, 51.5% yield, 98% purity) as yellow oil.

LCMS (AM3): rt=0.745 min, (569.1 [M+H]⁺), 98.2% purity.

### Synthesis of Intermediate 1.25

### methyl 5-((tert-butoxycarbonyl)(2-((tert-butoxycarbonyl)(4-((tert-butoxycarbonyl)((2-chloro-[1,1′-biphenyl]-4-yl)methyl)amino)butyl)amino)ethyl)amino)benzo[c][2,6]naphthyridine-8-carboxylate 1.88

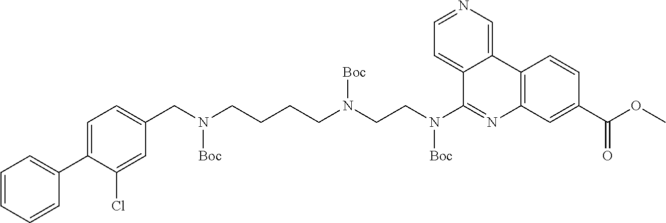 [SMILES: COC(=O)c1ccc2c(c1)nc(N(CCN(CCCCN(Cc1ccc(-c3ccccc3)c(Cl)c1)C(=O)OC(C)(C)C)C(=O)OC(C)(C)C)C(=O)OC(C)(C)C)c1ccncc12]

To a mixture of compound 1.8 (190 mg, 0.243 mmol, TFA salt), DMAP (30 mg, 0.246 mmol) and TEA (400 mg, 3.95 mmol) in THF (10 mL) was added (Boc) 20 (200 mg, 0.916 mmol) at RT. The mixture was heated to reflux and stirred for 18 h. The mixture was concentrated in vacuo to afford compound 1.88 (0.5 g) as a light brown solid, which was used directly without purification.

LCMS (AM2): rt=1.012 min, (868.4 [M+H]⁺), 87.2% purity.

### 5-((tert-butoxycarbonyl)(2-((tert-butoxycarbonyl)(4-((tert-butoxycarbonyl)((2-chloro-[1,1′-biphenyl]-4-yl)methyl)amino)butyl)amino)ethyl)amino)benzo[c][2,6]naphthyridine-8-carboxylic acid 1.89

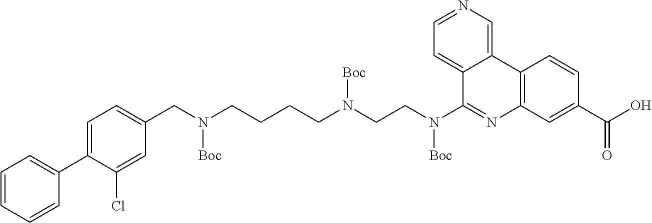 [SMILES: CC(C)(C)OC(=O)N(CCCCN(Cc1ccc(-c2ccccc2)c(Cl)c1)C(=O)OC(C)(C)C)CCN(C(=O)OC(C)(C)C)c1nc2cc(C(=O)O)ccc2c2cnccc12]

A mixture of compound 1.88 (0.5 g) and LiOH. H₂O (625.00 mg, 14.89 mmol) in THF (5 mL), H₂O (5 mL) and MeOH (5 mL) was stirred at RT for 18 h. The pH of the reaction mixture was adjusted to pH7 with aq. HCl (1 N) and extracted with EA (20 mL×3). The combined organic phase was dried (Na₂SO₄) and concentrated in vacuo to give compound 1.89 (200 mg) as a light brown solid, used directly without purification.

LCMS (AM2): rt=0.961 min, (854.3 [M+H]⁺), 74.9% purity.

### tert-butyl (4-((tert-butoxycarbonyl)(2-((tert-butoxycarbonyl)(8-carbamoylbenzo[c][2,6]naphthyridin-5-yl)amino)ethyl)amino)butyl)((2-chloro-[1,1′-biphenyl]-4-yl)methyl)carbamate 1.25

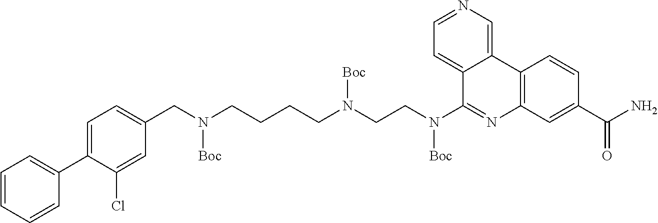 [SMILES: CC(C)(C)OC(=O)N(CCCCN(Cc1ccc(-c2ccccc2)c(Cl)c1)C(=O)OC(C)(C)C)CCN(C(=O)OC(C)(C)C)c1nc2cc(C(N)=O)ccc2c2cnccc12]

To a mixture of compound 1.89 (200 mg, 0.23 mmol), NH₄Cl (400 mg, 7.48 mmol), HOBt (160 mg, 1.18 mmol) and DIPEA (1 mL, 5.74 mmol) in DMF (20 mL) was added EDCI (230 mg, 1.20 mmol) at RT. The reaction mixture was heated to 70° C. and stirred for 8 h. The reaction mixture was added to H₂O (100 mL), the mixture extracted with EA (30 mL×2), the combined organic phase washed (brine, 30 mL), dried (Na₂SO₄), filtered and concentrated in vacuo to afford compound 1.25 (200 mg) as a yellow gum, which was used directly without further purification.

LCMS (AM1): rt=0.877 min, (753.3 [M+H]⁺), 88% purity

### Synthesis of Intermediate C

### methyl 5-((3-((tert-butoxycarbonyl)amino)propyl)amino)benzo[c][2,6]naphthyridine-8-carboxylate, 1.26

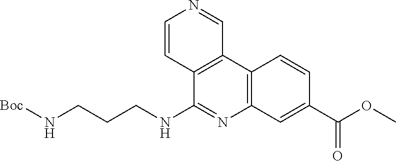 [SMILES: COC(=O)c1ccc2c(c1)nc(NCCCNC(=O)OC(C)(C)C)c1ccncc12]

A mixture of compound 1.1 (10 g, 36.67 mmol), tert-butyl N-(3-aminopropyl) carbamate (8.31 g, 47.67 mmol) and DIPEA (16 mL, 91.86 mmol) in DMSO (150 mL) was heated to 70° C. and stirred for 15 h. The reaction mixture was added to H₂O (1 L), the aq. mixture extracted with EA (500 mL×2), the combined organic phase washed (brine, 500 mL), dried (Na₂SO₄), filtered and concentrated in vacuo. The crude product was triturated in EtOH (100 mL) and then filtered and dried under vacuum to afford compound 1.26 (10.2 g, 65.7% yield) as a yellow solid.

LCMS (AM1): rt=0.833 min, (411.2 [M+H]⁺), 95.6% purity.

### 5-((3-((tert-butoxycarbonyl)amino)propyl)amino)benzo[c][2,6]naphthyridine-8-carboxylic acid, 1.27

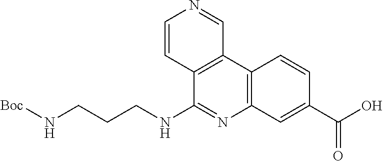 [SMILES: CC(C)(C)OC(=O)NCCCNc1nc2cc(C(=O)O)ccc2c2cnccc12]

To a mixture of compound 1.26 (10.2 g, 24.10 mmol) in THF (50 mL) and H₂O (50 mL) was added LiOH·H₂O (3.46 g, 82.46 mmol) at RT. The reaction mixture was heated to 40° C. and stirred for 15 h. The reaction mixture was added to H₂O (200 mL) and the pH adjusted to pH3 with aq. HCl (1 M). The resulting precipitate was collected by filtration and dried in vacuo to afford compound 1.27 (9.5 g, 98.1% yield) as a yellow solid, which was used directly without further purification.

LCMS (AM1): rt=0.765 min, (397.2 [M+H]⁺), 97.5% purity.

### tert-butyl (3-((8-carbamoylbenzo[c][2,6]naphthyridin-5-yl)amino)propyl)carbamate, 1.28

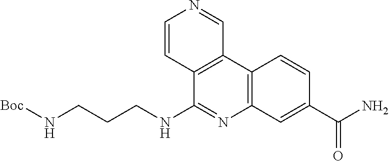 [SMILES: CC(C)(C)OC(=O)NCCCNc1nc2cc(C(N)=O)ccc2c2cnccc12]

To a mixture of compound 1.27 (9.5 g, 23.65 mmol), NH₄Cl (7.6 g, 142.08 mmol), HOBt (6.39 g, 47.30 mmol) and DIPEA (16.5 mL, 94.73 mmol) in DMF (150 mL) was added EDCI (9.07 g, 47.30 mmol) at RT. The reaction mixture was then heated to 70° C. and stirred for 14 h. The reaction mixture was added to H₂O (600 mL), the mixture extracted with EA (200 mL×5), the combined organic phase washed (brine, 500 mL×2), dried (Na₂SO₄), filtered and concentrated in vacuo to afford compound 1.28 (9.1 g, 94.4% yield) as a yellow solid, which was used directly without further purification.

LCMS (AM1): rt=0.756 min, (396.2 [M+H]⁺), 96.77% purity.

### 5-((3-aminopropyl)amino)benzo[c][2,6]naphthyridine-8-carboxamide, 1.29

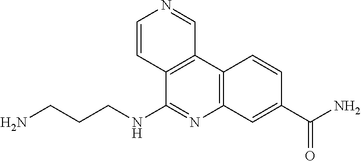 [SMILES: NCCCNc1nc2cc(C(N)=O)ccc2c2cnccc12]

To a mixture of compound 1.28 (9.1 g, 23.01 mmol) in 1,4-dioxane (50 mL) was added a solution of HCl in 1,4-dioxane (4 M, 50 mL) dropwise at RT, and the mixture stirred for 15 h. The reaction mixture was concentrated in vacuo to afford compound 1.29 (9.2 g, HCl salt) as a yellow solid, which was used without further purification.

### benzyl (3-((3-((8-carbamoylbenzo[c][2,6]naphthyridin-5-yl)amino)propyl)amino)-3-oxopropyl)carbamate, 1.30

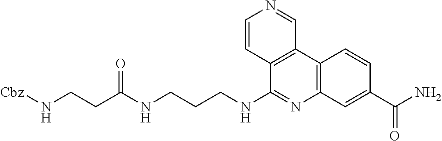 [SMILES: NC(=O)c1ccc2c(c1)nc(NCCCNC(=O)CCNC(=O)OCc1ccccc1)c1ccncc12]

To a mixture of compound 1.29 (8.2 g, 24.71 mmol, HCl salt) and 3-(benzyloxycarbonylamino)propanoic acid (6.18 g, 27.68 mmol) in DCM (100 mL) was added TEA (16 mL) followed by T₃P (19 mL, 50% purity, 31.95 mmol) at 30° C. and the reaction mixture was stirred for 1 h. The reaction mixture was concentrated in vacuo. The residue was triturated in H₂O (200 mL) and filtered, and the obtained filter cake was triturated again in ACN (100 mL) and filtered. The filter cake was dried under vacuum to afford compound 1.30 (8.5 g) as a yellow solid, which was used directly without further purification.

LCMS (AM2): rt=0.748 min, (501.2 [M+H]⁺), 96.77% purity.

### 5-((3-(3-aminopropanamido)propyl)amino)benzo[c][2,6]naphthyridine-8-carboxamide, Intermediate C

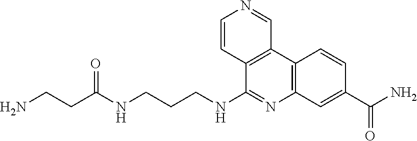 [SMILES: NCCC(=O)NCCCNc1nc2cc(C(N)=O)ccc2c2cnccc12]

A mixture of compound 1.30 (1 g, 2.00 mmol) and Pd/C (100 mg, 2.00 mmol, 10% Pd/C) in MeOH (50 mL) and THF (50 mL) was stirred under a H₂ balloon at RT for 22 h. The reaction mixture was filtered through a pad of Celite® and the filtrate concentrated in vacuo to afford Intermediate C (760 mg, 81% yield, 78% purity) as a yellow solid, which was used directly without further purification.

LCMS (AM1): rt=0.669 min, (367.1 [M+H]⁺), 76.3% purity.

### Synthesis of Intermediate 1.32

### 3-chloro-4-cyclobutoxybenzaldehyde 1.32

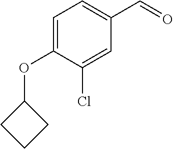 [SMILES: O=Cc1ccc(OC2CCC2)c(Cl)c1]

A mixture of bromocyclobutane (0.25 mL, 2.65 mmol), 3-chloro-4-hydroxybenzaldehyde (200 mg, 1.28 mmol) and K₂CO₃ (440 mg, 3.18 mmol) in DMF (10 mL) was stirred at 80° C. for 15 h. The reaction mixture was poured into H₂O (60 mL), the mixture extracted with EA (20 mL×3), the combined organic phase washed with brine (30 mL), dried over Na₂SO₄, filtered and concentrated in vacuo to give the crude product which was purified (PM11) to afford compound 1.32 (134 mg, 49.8% yield) as a colourless oil.

LCMS (AM1): rt=0.969 min, (211.0 [M+H]⁺), 66.6% purity.

### Synthesis of Intermediate 1.33

### 3-chloro-4-(cyclopentyloxy)benzaldehyde 1.33

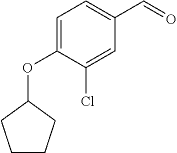 [SMILES: O=Cc1ccc(OC2CCCC2)c(Cl)c1]

A mixture of bromocyclopentane (0.274 mL, 2.56 mmol,), 3-chloro-4-hydroxy-benzaldehyde (200 mg, 1.28 mmol) and K₂CO₃ (441 mg, 3.19 mmol) in DMF (10 mL) was stirred at 80° C. for 15 h. The reaction mixture was poured onto H₂O (60 mL), the resulting mixture was extracted with EA (20 mL×3), the combined organic phase washed with brine (30 mL), dried over Na₂SO₄, filtered and concentrated in vacuo. The crude product was purified (PM11) to afford compound 1.33 (270 mg, 93.9% yield) as a colourless oil.

LCMS (AM3): rt=1.017 min, (266.0 [M+H₂O+Na]⁺), 96.9% purity.

### Synthesis of Intermediate 1.37

### 2-(((2-chloro-[1,1′-biphenyl]-4-yl)methyl)amino)ethanol 1.34

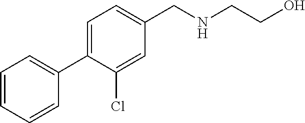 [SMILES: OCCNCc1ccc(-c2ccccc2)c(Cl)c1]

A mixture of 3-chloro-4-phenylbenzaldehyde (CAS 57592-44-6, 1 g, 4.62 mmol) and 2-aminoethanol (846 mg, 13.85 mmol) in MeOH (20 mL) was stirred at RT for 1 h, NaBH(OAc)₃ (1.96 g, 9.23 mmol) was added and the reaction mixture was stirred for 4 h. The reaction mixture was poured onto H₂O (100 mL), the mixture extracted with EA (60 mL×2), the combined organic phase washed (brine, 100 mL), dried (Na₂SO₄), filtered and concentrated in vacuo to afford compound 1.34 (1.07 g, 88.6% yield) as a colourless oil, which was used directly without purification.

LCMS (AM3): rt=0.745 min, (261.9 [M+H]⁺), 85.8% purity.

### tert-butyl ((2-chloro-[1,1′-biphenyl]-4-yl)methyl)(2-hydroxyethyl)carbamate 1.35

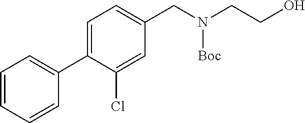 [SMILES: CC(C)(C)OC(=O)N(CCO)Cc1ccc(-c2ccccc2)c(Cl)c1]

To a mixture of compound 1.34 (1.07 g, 4.09 mmol) and NaHCO₃ (687 mg, 8.18 mmol) in THF (10 mL) and H₂O (10 mL) was added Boc₂O (1.07 g, 4.91 mmol) at RT, the reaction mixture stirred for 16 h. The reaction mixture was poured onto H₂O (100 mL), extracted with EA (50 mL×2), the combined organic phase washed (brine, 100 mL), dried (Na₂SO₄), filtered and concentrated in vacuo. The residue was purified (PM6) to afford compound 1.35 (1.24 g, 83.8% yield) as a colourless oil.

LCMS (AM3): rt=0.989 min, (384.2 [M+Na]⁺), 83.3% purity.

### tert-butyl ((2-chloro-[1,1′-biphenyl]-4-yl)methyl)(2-oxoethyl)carbamate 1.36

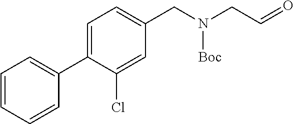 [SMILES: CC(C)(C)OC(=O)N(CC=O)Cc1ccc(-c2ccccc2)c(Cl)c1]

To a solution of compound 1.35 (780 mg, 2.16 mmol) in DCM (15 mL) was added DMP (1.1 g, 2.59 mmol) in portions at RT, the reaction mixture was stirred for 18 h. The reaction mixture was filtered and the filtrate was concentrated in vacuo. The residue was purified (PM11) to afford compound 1.36 (660 mg) as a colourless oil.

¹H NMR (400 MHz, DMSO-d₆) δ: 9.58-9.54 (m, 1H), 7.47-7.32 (m, 7H), 7.23-7.16 (m, 1H), 4.57-4.16 (m, 2H), 3.89-3.50 (m, 2H), 1.52-1.50 (m, 9H) ppm.

### Methyl 5-((2-((2-((tert-butoxycarbonyl) ((2-chloro-[1,1′-biphenyl]-4-yl)methyl)amino)ethyl)amino)ethyl)amino)benzo[c][2,6]naphthyridine-8-carboxylate 1.37

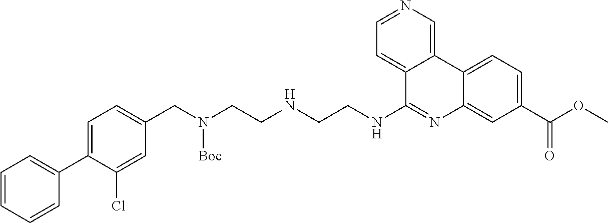 [SMILES: COC(=O)c1ccc2c(c1)nc(NCCNCCN(Cc1ccc(-c3ccccc3)c(Cl)c1)C(=O)OC(C)(C)C)c1ccncc12]

To a mixture of compound 1.36 (112 mg, 0.311 mmol) and NaOAc (102 mg, 1.24 mmol) in MeOH (5 mL) was added Intermediate A (170 mg, 0.311 mmol, HCl salt) followed by NaBH₃CN (80 mg, 1.27 mmol) at RT, the reaction was stirred for 16 h. The reaction mixture was filtered and concentrated in vacuo to give a residue, which was purified (PM27) to afford compound 1.37 (40 mg, 20% yield) as a yellow solid.

LCMS (AM1): rt=0.809 min, (640.2 [M+H]⁺), 36.4% purity.

### Synthesis of Intermediate D

### 3-(((2-chloro-[1,1′-biphenyl]-4-yl)methyl)amino)propanoic acid, 1.39

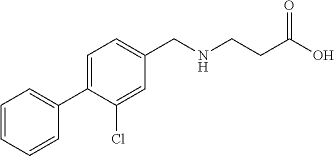 [SMILES: O=C(O)CCNCc1ccc(-c2ccccc2)c(Cl)c1]

A solution of 3-chloro-4-phenylbenzaldehyde (CAS 57592-44-6, 10 g, 46.15 mmol) and 3-aminopropanoic acid (12.34 g, 138.46 mmol) in MeOH (200 mL) was stirred at 34° C. for 1 h. NaBH(OAc)₃ (19.56 g, 92.31 mmol) was added slowly and the reaction mixture was stirred at 34° C. for 14 h. The reaction mixture was filtered and concentrated in vacuo to give a residue that was purified (PM28) to afford compound 1.39 (11.9 g, 41.07 mmol, 89% yield) as a colourless oil.

LCMS (AM3): rt=0.758 min, (290.1 [M+H]⁺), 50% purity.

### 3-((tert-butoxycarbonyl)((2-chloro-[1,1′-biphenyl]-4-yl)methyl)amino)propanoic acid, Intermediate D

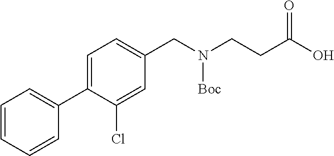 [SMILES: CC(C)(C)OC(=O)N(CCC(=O)O)Cc1ccc(-c2ccccc2)c(Cl)c1]

To a solution of compound 1.39 (11.9 g, 41.07 mmol) in THF (75 mL) and H₂O (75 mL) was added sodium bicarbonate (5.18 g, 61.60 mmol) and (Boc) 20 (10.76 g, 49.28 mmol) sequentially at 30° C., the mixture was stirred for 2 h. The reaction mixture was filtered and concentrated, and the residue was purified (PM28) to afford Intermediate D (13.8 g, 35.40 mmol, 86.2% yield) as a yellow oil.

LCMS (AM3): rt=0.997 min, (412.1 [M+Na]⁺). 92% purity.

### Synthesis of Intermediate 1.44

### Methyl 5-((3-((tert-butoxycarbonyl)(methyl)amino)propyl)amino)benzo[c][2,6]naphthyridine-8-carboxylate 1.40

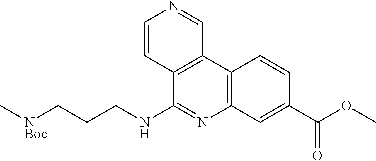

To a solution of compound 1.1 (500 mg, 1.83 mmol) in DMSO (10 mL) was added DIPEA (473 mg, 3.67 mmol) and tert-butyl (3-aminopropyl)(methyl)carbamate (517 mg, 2.75 mmol) at RT. The mixture was heated to 75° C. and stirred for 12 h. The mixture was poured into 50 mL of iced-H₂O, the precipitate was collected by filtration and dried under vacuum to afford compound 1.40 (750 mg, 1.43 mmol, 77.9% yield) as a red solid.

LCMS (AM3): rt=0.757 min, (425.2 [M+H]⁺), 80.9% purity.

### 5-((3-((tert-butoxycarbonyl)(methyl)amino)propyl)amino)benzo[c][2,6]naphthyridine-8-carboxylic acid 1.41

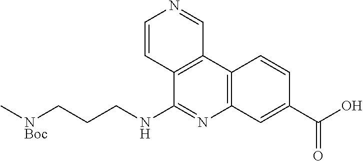 [SMILES: CN(CCCNc1nc2cc(C(=O)O)ccc2c2cnccc12)C(=O)OC(C)(C)C]

To a solution of compound 1.40 (740 mg, 1.41 mmol) in THF (10 mL), MeOH (1 mL) and H₂O (1 mL) was added NaOH (564 mg, 14.10 mmol) at RT. The mixture was heated to 50° C. and stirred for 2 h. The reaction mixture was concentrated in vacuo and purified (PM29) to afford compound 1.41 (550 mg, 1.31 mmol, 93.1% yield) as a red solid.

LCMS (AM3): rt=0.782 min, (411.4 [M+H]⁺), 98.2% purity.

### tert-butyl (3-((8-carbamoylbenzo[c][2,6]naphthyridin-5-yl)amino)propyl)(methyl)carbamate 1.42

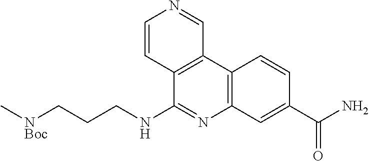 [SMILES: CN(CCCNc1nc2cc(C(N)=O)ccc2c2cnccc12)C(=O)OC(C)(C)C]

To a solution of compound 1.41 (550 mg, 1.34 mmol) in THF (10 mL) was added DIPEA (519 mg, 4.02 mmol), NH₄Cl (143 mg, 2.68 mmol) and HATU (662 mg, 1.74 mmol) sequentially at 25° C., the reaction mixture was stirred for 12 h. The reaction mixture was poured into 50 mL of iced-H₂O and the precipitate was collected by filtration and dried under vacuum to afford compound 1.42 (400 mg, 0.93 mmol, 69.4% yield) as a red solid.

LCMS (AM3): rt=0.697 min, (410.3 [M+H]⁺), 95.3% purity.

### 5-((3-(methylamino)propyl)amino)benzo[c][2,6]naphthyridine-8-carboxamide 1.43

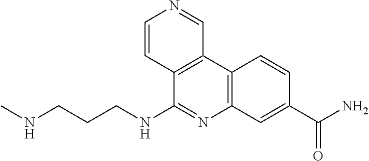 [SMILES: CNCCCNc1nc2cc(C(N)=O)ccc2c2cnccc12]

To a solution of compound 1.42 (380 mg, 0.92 mmol) in 1,4-dioxane (10 mL) was added a solution of HCl in dioxane (4 M, 5 mL) at 0° C. The reaction mixture was warmed to 25° C. and stirred for 0.5 h. The reaction mixture was concentrated in vacuo to afford compound 1.43 (400 mg, 0.89 mmol, 96.6% yield, HCl salt) as a light yellow solid, which was used directly without further purification.

LCMS (AM5): rt=0.621 min, (310.2 [M+H]⁺), 77.5% purity.

### tert-butyl (3-((3-((8-carbamoylbenzo[c][2,6]naphthyridin-5-yl)amino)propyl)(methyl)amino)-3-oxopropyl)((2-chloro-[1,1′-biphenyl]-4-yl)methyl)carbamate 1.44

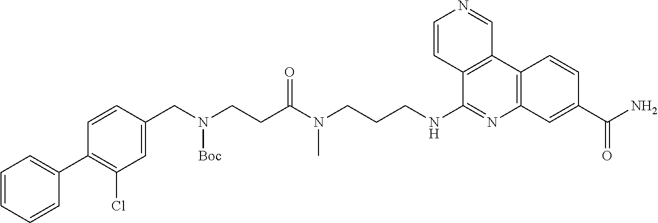 [SMILES: CN(CCCNc1nc2cc(C(N)=O)ccc2c2cnccc12)C(=O)CCN(Cc1ccc(-c2ccccc2)c(Cl)c1)C(=O)OC(C)(C)C]

To a solution of Intermediate D (100 mg, 0.25 mmol) in THF (10 mL) was added HATU (117 mg, 0.31 mmol), compound 1.43 (137 mg, 0.31 mmol, HCl salt) and DIPEA (99 mg, 0.77 mmol) sequentially at 25° C., the reaction mixture was stirred for 12 h. The reaction mixture was concentrated in vacuo and purified (PM30) to afford compound 1.44 (130 mg, 0.19 mmol, 74.4% yield) as a yellow solid.

LCMS (AM3): rt=0.913 min, (681.3 [M+H]⁺), 100% purity.

### Synthesis of Intermediate 1.49

### 4-formyl-[1,1′-biphenyl]-2-carbonitrile 1.49

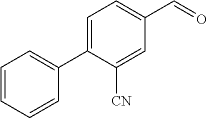 [SMILES: N#Cc1cc(C=O)ccc1-c1ccccc1]

To a mixture of phenylboronic acid (174 mg, 1.43 mmol) and 2-bromo-5-formylbenzonitrile (200 mg, 952.26 μmol) in 1,4-dioxane (1 mL) and H₂O (0.1 mL) was added K₂CO₃ (329.02 mg, 2.38 mmol) and XPhos-Pd-G3 (80.60 mg, 95.23 μmol), sequentially at 25° C. under N₂. The mixture was then heated to 90° C. and stirred for 12 h. The mixture was concentrated in vacuo and purified (PM32) to afford compound 1.49 (100 mg, 477.74 μmol, 50.1% yield, 99% purity) as a white solid.

LCMS (AM3): rt=0.911 min, (208.0 [M+H]⁺), 99.0% purity.

¹H NMR (400 MHz, CHCl₃-d) δ: 10.09 (s, 1H), 8.28 (d, J=1.6 Hz, 1H), 8.16 (dd, J=1.6, 8.4 Hz, 1H), 7.73 (d, J=8.4 Hz, 1H), 7.66-7.59 (m, 2H), 7.59-7.53 (m, 3H) ppm.

### Synthesis of Intermediate 1.51

### 6-(trifluoromethoxy)quinoline-2-carbaldehyde 1.51

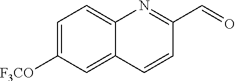 [SMILES: O=Cc1ccc2cc(OC(F)(F)F)ccc2n1]

To a mixture of 2-methyl-6-(trifluoromethoxy) quinoline (ref: U.S. Pat. No. 6,288,075) (100 mg, 440.18 μmol) in 1,4-dioxane (5 mL) was added selenium dioxide (195.37 mg, 1.76 mmol) at 25° C. under N₂, the mixture was heated to 80° C. and stirred for 12 h. The reaction mixture was concentrated in vacuo and purified (PM33) to afford compound 1.51 (20 mg, 82.93 μmol, 18.8% yield) as yellow oil.

LCMS (AM3): rt=0.885 min, (242.1 [M+H]⁺), 64.18% purity.

¹H NMR (400 MHz, CHCl₃-d) δ: 10.23 (s, 1H), 8.36-8.30 (m, 2H), 8.09 (d, J=8.8 Hz, 1H), 7.74-7.68 (m, 2H) ppm.

### Synthesis of Intermediate 1.67

### (4-Chloro-5-phenyl-1H-pyrazol-3-yl)methanol 1.66

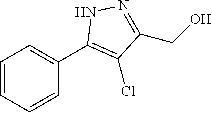 [SMILES: OCc1n[nH]c(-c2ccccc2)c1Cl]

To a mixture of 5-phenyl-1H-pyrazol-3-yl methanol (800 mg, 4.59 mmol) (Journal of Medicinal Chemistry, 1998, 41 (13), 2390-2410) in ACN (10 mL) was added NCS (919.87 mg, 6.89 mmol) under N₂ at 25° C. The mixture was stirred at 25° C. for 12 h. The mixture was filtered and the filtrate was concentrated in vacuo to give a residue. The obtained residue was purified (PM34) to afford compound 1.66 (250 mg, 1.14 mmol, 24.8% yield, 95% purity) as a white solid.

LCMS (AM3): rt=0.813 min, (209.1 [M+H]⁺), 94.39% purity.

### 4-Chloro-5-phenyl-1H-pyrazole-3-carbaldehyde 1.67

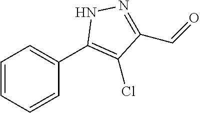 [SMILES: O=Cc1n[nH]c(-c2ccccc2)c1Cl]

To a mixture of compound 1.66 (250 mg, 1.20 mmol) in DCM (10 mL) was added manganese (IV) oxide (1.04 g, 11.98 mmol) under N₂ at 25° C. The mixture was stirred at 25° C. for 12 h. The mixture was filtered and the filtrate was concentrated in vacuo to give a residue which was further purified (PM35) to afford compound 1.67 (60 mg, 281.67 μmol, 23.5% yield, 97% purity) as a white solid.

LCMS (AM3): rt=0.858 min, (207.0 [M+H]⁺), 96.49% purity.

### Synthesis of Intermediate F and Intermediate G

### 5-((2-Aminoethyl)amino)benzo[c][2,6]naphthyridine-8-carboxamide, Intermediate F

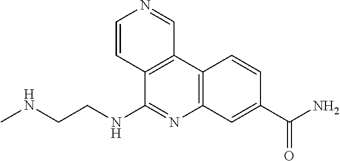 [SMILES: CNCCNc1nc2cc(C(N)=O)ccc2c2cnccc12]

A mixture of compound 1.13 (14.4 g, 37.75 mmol) and TFA (1.35 mol, 100.00 mL) in DCM (50 mL) was stirred at RT for 18 h. The reaction mixture was concentrated in vacuo to afford Intermediate F (21.2 g, TFA salt) as a yellow solid, which was used directly without further purification.

LCMS (AM3): rt=0.666 min, (282.0 [M+H]⁺), 100% purity.

### Benzyl (4-((2-((8-carbamoylbenzo[c][2,6]naphthyridin-5-yl)amino)ethyl)amino)butyl)carbamate, 1.69

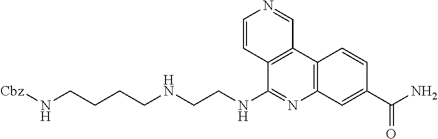 [SMILES: NC(=O)c1ccc2c(c1)nc(NCCNCCCCNC(=O)OCc1ccccc1)c1ccncc12]

A mixture of Intermediate F (21.2 g, 53.63 mmol, TFA salt), benzyl (4-oxobutyl)carbamate (7 g, 31.64 mmol) (ref: WO2018170306) and DIPEA (126.31 mmol, 21.99 mL) in MeOH (250 mL) was stirred at RT for 14 h, then NaBH(OAc)₃ (20 g, 94.37 mmol) was added. The reaction mixture was stirred at RT for 4 h. The reaction mixture was filtered and the filtrate was concentrated in vacuo. The residue was purified (PM34) to afford compound 1.69 (3.7 g, 19.5% yield, TFA salt) as a yellow solid.

LCMS (AM3): rt=0.750 min, (487.2 [M+H]⁺), 100% purity.

### tert-Butyl (4-(((benzyloxy)carbonyl)amino)butyl)(2-((8-carbamoylbenzo[c][2,6]naphthyridin-5-yl)amino)ethyl)carbamate, 1.70

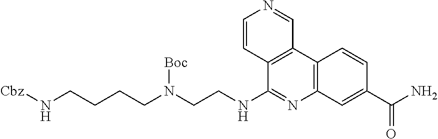 [SMILES: CC(C)(C)OC(=O)N(CCCCNC(=O)OCc1ccccc1)CCNc1nc2cc(C(N)=O)ccc2c2cnccc12]

A mixture of compound 1.69 (3.7 g, 6.16 mmol, TFA salt), TEA (24.43 mmol, 3.4 mL) and Boc₂O (6.53 mmol, 1.5 mL) in MeOH (40 mL) was stirred at RT for 18 h. The reaction mixture was concentrated in vacuo. The obtained residue was dissolved in EA and MeOH (v:v=10:1, 100 mL), and the resulting solution was washed with H₂O (100 mL) and brine (100 mL×2). The organic layer was dried (Na₂SO₄), filtered and concentrated in vacuo to afford compound 1.70 (1.23 g, 2.10 mmol, 34% yield) as a yellow solid, which was used directly without further purification.

LCMS (AM3): rt=0.839 min, (587.3 [M+H]⁺), 87.7% purity.

### tert-Butyl (4-aminobutyl)(2-((8-carbamoylbenzo[c][2,6]naphthyridin-5-yl)amino)ethyl)carbamate, Intermediate G

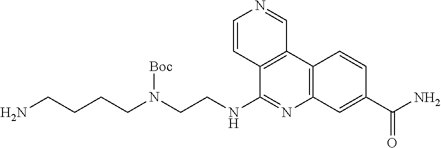 [SMILES: CC(C)(C)OC(=O)N(CCCCN)CCNc1nc2cc(C(N)=O)ccc2c2cnccc12]

A mixture of compound 1.70 (1.23 g, 2.10 mmol) and 10% Pd/C (100 mg) in MeOH (10 mL) and aq. ammonia (1 mL) was hydrogenated under hydrogen at 15 psi at RT for 72 h. The reaction mixture was filtered and the filtrate was concentrated in vacuo to afford Intermediate G (810 mg, 85.4% yield) as a yellow solid, which was used directly without purification.

LCMS (AM3): rt=0.700 min, (453.1 [M+H]⁺), 83% purity.

### Synthesis of Intermediate 1.74

### (2-((2-Bromophenethoxy)methoxy)ethyl)trimethylsilane 1.72

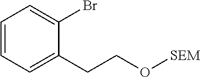 [SMILES: C[Si](C)(C)CCOCOCCc1ccccc1Br]

A mixture of 2-(2-bromophenyl) ethanol (500 mg, 2.49 mmol), DIPEA (5.74 mmol, 1 mL) and (2-(chloromethoxy)ethyl)trimethylsilane (2.83 mmol, 0.5 mL) in DCM (10 mL) was stirred at 40° C. for 18 h. The reaction mixture was concentrated in vacuo and purified (PM11) to afford compound 1.72 (550 mg, 66.8% yield) as a colourless oil.

¹H NMR (400 MHz, MeOH-d₄) δ: 7.54 (dd, J=8.0 Hz, 0.8 Hz, 1H), 7.37-7.31 (m, 1H), 7.30-7.24 (m, 1H), 7.13-7.09 (m, 1H), 4.63 (s, 2H), 3.76 (t, J=6.8 Hz, 2H), 3.55-3.51 (t, 2H), 3.03 (t, J=6.8 Hz, 2H), 0.87 (t, J=8.0 Hz, 2H), 0.00 (s, 9H) ppm.

### 2-Chloro-2′-(2-((2-(trimethylsilyl)ethoxy)methoxy)ethyl)-[1,1′-biphenyl]-4-carbaldehyde 1.73

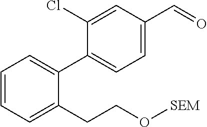 [SMILES: C[Si](C)(C)CCOCOCCc1ccccc1-c1ccc(C=O)cc1Cl]

A mixture of compound 1.72 (150 mg, 0.453 mmol), (2-chloro-4-formylphenyl)boronic acid (CAS 1063712-34-4, 125 mg, 0.678 mmol) and K₂CO₃ (125 mg, 0.904 mmol) in 1,4-dioxane (8 mL) and H₂O (2 mL) was degassed and purged with N₂ three times, then Pd(dppf)Cl₂ (33 mg, 0.045 mmol) was added at RT. The reaction mixture was heated to 80° C. and stirred for 16 h. The reaction mixture was diluted with EA (40 mL) and the organic phase was washed (brine, 40 mL), dried (Na₂SO₄), filtered and concentrated in vacuo. The residue was purified (PM11) to afford compound 1.73 (130 mg) as a colourless oil.

¹H NMR (400 MHz, MeOH-d₄) δ: 10.02 (s, 1H), 8.04 (d, J=1.6 Hz, 1H), 7.91 (dd, J=8.0 Hz, 2.0 Hz, 1H), 7.51 (d, J=8.0 Hz, 1H), 7.40-7.27 (m, 3H), 7.12-7.08 (m, 1H), 4.49 (s, 2H), 3.56-3.53 (m, 2H), 3.46-3.41 (m, 2H), 2.75-2.64 (m, 2H), 0.82-0.77 (m, 2H), −0.03 (s, 9H) ppm.

### tert-Butyl (2-((8-carbamoylbenzo[c][2,6]naphthyridin-5-yl)amino)ethyl)(4-(((2-chloro-2′-(2-((2-(trimethylsilyl)ethoxy)methoxy)ethyl)-[1,1′-biphenyl]-4-yl)methyl)amino)butyl)carbamate 1.74

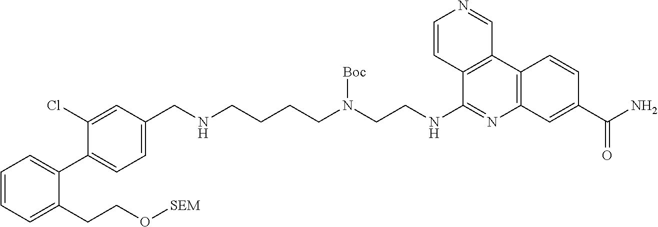 [SMILES: CC(C)(C)OC(=O)N(CCCCNCc1ccc(-c2ccccc2CCOCOCC[Si](C)(C)C)c(Cl)c1)CCNc1nc2cc(C(N)=O)ccc2c2cnccc12]

A mixture of compound 1.73 (130 mg, 0.333 mmol), Intermediate G (80 mg, 0.177 mmol) and DIPEA (0.574 mmol, 0.1 mL) in MeOH (10 mL) was stirred at 30° C. for 16 h, then NaBH(OAc)₃ (150 mg, 0.708 mmol) was added. The reaction mixture was stirred at 30° C. for another 2 h. The reaction mixture was filtered and the filtrate was concentrated in vacuo. The crude product was purified (PM36) to afford compound 1.74 (117 mg, 70.3% yield, TFA salt) as a yellow solid.

LCMS (AM4): rt=0.829 min, (827.3 [M+H]⁺), 97.29% purity.

The following intermediates in Table 1 were made with non-critical changes or substitutions to the exemplified procedure in Intermediate 1.74, that would be understood by one skilled in the art using Intermediate G and suitable aldehyde.

TABLE 1

| Intermediate | | | |
|---|---|---|---|
| No. | Chemical IUPAC name | Aldehyde | Analytical |
| 1.104 | (4-(((4-((tert-Butoxycarbonyl)(2-((8- carbamoylbenzo[c][2,6]naphthyridin-5- yl)amino)ethyl)amino)butyl)amino) methyl)-2-chlorophenyl)boronic acid | 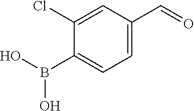 [SMILES: O=Cc1ccc(B(O)O)c(Cl)c1] | LCMS (AM3): rt = 0.734 min, (621.2 [M + H]⁺), 99.1% purity. |
| | | (2-chloro-4- | Purification |
| | | formylphenyl)boronic | Method |
| | | acid CAS | 42 |
| | | 1063712-34-4 | |
| 1.130 | tert-Butyl (2-((8- carbamoylbenzo[c][2,6]naphthyridin-5- yl)amino)ethyl)(4-(((6-methyl-5- phenylpyridin-2- yl)methyl)amino)butyl)carbamate | 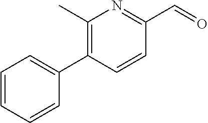 | LCMS (AM3): rt = 0.804 min, (634.2 [M + H]⁺), 100% purity Purification |
| | | 6-Methyl-5- | Method |
| | | phenylpicolinaldehyde | 51 |
| | | CAS 2921724- | |
| | | 20-9 | |
| 1.167 | tert-butyl (2-((8- carbamoylbenzo[c][2,6]naphthyridin-5- yl)amino)ethyl)(4-(((4-methyl-5- phenylpyridin-2- yl)methyl)amino)butyl)carbamate1 | 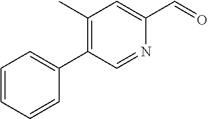 | LCMS (AM3): rt = 0.803 min, (634.2 [M + H]⁺), 100% purity. Purification |
| | | 4-methyl-5- | Method |
| | | phenylpicolinaldehyde | 51 |
| | | 1.166 | |

### Synthesis of Intermediate 1.76

### tert-Butyl (2-((8-carbamoylbenzo[c][2,6]naphthyridin-5-yl)amino)ethyl) (4-(((5-(trifluoromethyl)-1H-indol-2-yl)methyl)amino)butyl)carbamate 1.76

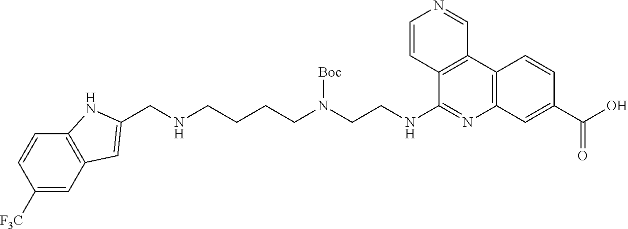 [SMILES: CC(C)(C)OC(=O)N(CCCCNCc1cc2cc(C(F)(F)F)ccc2[nH]1)CCNc1nc2cc(C(=O)O)ccc2c2cnccc12]

A mixture of 5-(trifluoromethyl)-1H-indole-2-carbaldehyde (CAS 1367793-79-0, 84.92 mg, 187.66 μmol) and Intermediate G (40 mg, 187.66 μmol) in MeOH (5 mL) was stirred at 25° C. for 1 h, then NaBH(OAc)₃ (159.09 mg, 750.62 μmol) was added. The mixture was stirred at 25° C. for 11 h. The reaction mixture was concentrated in vacuo and purified (PM37) to afford compound 1.76 (100 mg, 152.38 μmol, 81.2% yield) as a yellow solid.

LCMS (AM3): rt=0.829 min, (650.3 [M+H]⁺), 99.86% purity.

The following intermediates in Table 2 were made with non-critical changes or substitutions to the exemplified procedure in Intermediate 1.76, that would be understood by one skilled in the art using Intermediate G and a suitable aldehyde.

TABLE 2

| Intermediate | | | |
|---|---|---|---|
| No | Chemical IUPAC name | Aldehyde | Analytical |
| 1.103 | tert-Butyl (2-((8- carbamoylbenzo[c][2,6] naphthyridin-5- yl)amino)ethyl)(4-((3- chloro-4- (trifluoromethoxy)benzyl) amino) butyl) carbamate | 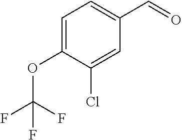 [SMILES: O=Cc1ccc(OC(F)(F)F)c(Cl)c1] | LCMS (AM3): rt = 0.777 min, (661.5 [M + H]⁺), 100% purity. Purification Method 41 |
| | | 3-chloro-4- | |
| | | (trifluoromethoxy)benz- | |
| | | aldehyde | |
| | | CAS 83279-39-4 | |
| 1.109 | tert-Butyl (2-((8- carbamoylbenzo[c][2,6] naphthyridin-5- yl)amino)ethyl)(4-(((2- chloro-2′-(2-((2- (trimethylsilyl)ethoxy) methoxy)ethoxy)-[1,1′- biphenyl]-4- yl)methyl)amino)butyl) carbamate | 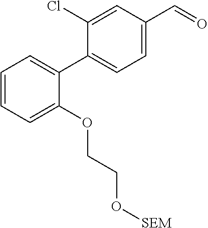 [SMILES: C[Si](C)(C)CCOCOCCOc1ccccc1-c1ccc(C=O)cc1Cl] | LCMS (AM3): rt = 0.910 min, (843.4 [M + H]⁺), 100% purity. Purification Method 43 |
| | | 2-Chloro-2′-(2-((2- | |
| | | (trimethylsilyl)ethoxy) | |
| | | methoxy)ethoxy)-[1,1′- | |
| | | biphenyl]-4-carbaldehyde | |
| | | 1.108 | |
| 1.110 | tert-Butyl (2-((8- carbamoylbenzo[c][2,6] naphthyridin-5- yl)amino)ethyl)(4-(((4- chloro-3-phenyl-1H- pyrazol-5- yl)methyl)amino)butyl) carbamate | 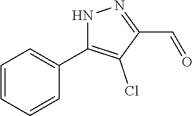 [SMILES: O=Cc1n[nH]c(-c2ccccc2)c1Cl] | LCMS (AM3): rt = 0.785 min, (643.5 [M + H]⁺), 99.8% purity Purification Method 44 |
| | | 4-Chloro-5-phenyl-1H- | |
| | | pyrazole-3-carbaldehyde | |
| | | 1.67 | |
| 1.111 | tert-Butyl (2-((8- carbamoylbenzo[c][2,6] naphthyridin-5- yl)amino)ethyl)(4-(((5- fluoro-1H-indol-2- yl)methyl)amino)butyl) carbamate | 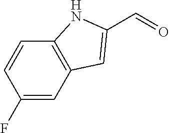 [SMILES: O=Cc1cc2cc(F)ccc2[nH]1] | LCMS (AM3): rt = 0.791 min, (600.4 [M + H]⁺), 99.8% purity. Purification Method 45 |
| | | 5-fluoro-1H-indole-2- | |
| | | carbaldehyde | |
| | | CAS 220943-23-7 | |
| 1.119 | tert-Butyl(4-(((1H-indol- 2- yl)methyl)amino)butyl) (2-((8- carbamoylbenzo[c][2,6] naphthyridin-5- yl)amino)ethyl)carbamate | 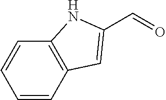 [SMILES: O=Cc1cc2ccccc2[nH]1] | LCMS (AM3): rt = 0.781 min, (582.2 [M + H]⁺), 99.3% purity. Purification Method 44 |
| | | 1H-indole-2-carbaldehyde | |
| | | CAS 19005-93-7 | |
| 1.120 | tert-Butyl(2-((8- carbamoylbenzo[c][2,6] naphthyridin-5- yl)amino)ethyl)(4-(((5- chloro-1H-indol-2- yl)methyl)amino)butyl) carbamate | 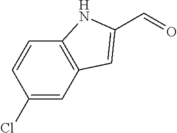 [SMILES: O=Cc1cc2cc(Cl)ccc2[nH]1] | LCMS (AM3): rt = 0.794 min, (616.1 [M + H]⁺), 100% purity. Purification Method 44 |
| | | 5-chloro-1H-indole-2- | |
| | | carbaldehyde | |
| | | CAS 53590-49-1 | |
| 1.121 | tert-Butyl (2-((8- carbamoylbenzo[c][2,6] naphthyridin-5- yl)amino)ethyl)(4-((3- chloro-4- cyclobutoxybenzyl) amino)butyl)carbamate | 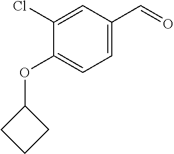 [SMILES: O=Cc1ccc(OC2CCC2)c(Cl)c1] | LCMS (AM3): rt = 0.821 min, (647.3 [M + H]⁺), 26.4% purity. Purification Method 47 |
| | | 3-chloro-4- | |
| | | cyclobutoxybenzaldehyde | |
| | | (ref: WO2022185041) | |
| 1.127 | tert-Butyl (2-((8- carbamoylbenzo[c][2,6] naphthyridin-5- yl)amino)ethyl)(4-(((5- chloro-6-phenylpyridin- 3- yl)methyl)amino)butyl) carbamate | 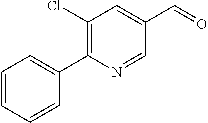 [SMILES: O=Cc1cnc(-c2ccccc2)c(Cl)c1] | LCMS (AM4): rt = 0.700 min, (654.3 [M + H]⁺), 80% purity. Purification Method 50 |
| | | 5-Chloro-6- | |
| | | phenylnicotinaldehyde | |
| | | CAS 1227582-64-0 | |
| 1.131 | tert-Butyl (2-((8- carbamoylbenzo[c][2,6] naphthyridin-5- yl)amino)ethyl)(4-(((2- fluoro-[1,1′-biphenyl]-4- yl)methyl)amino)butyl) carbamate | 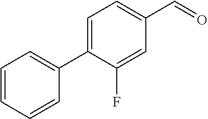 [SMILES: O=Cc1ccc(-c2ccccc2)c(F)c1] | LCMS (AM3): rt = 0.786 min, (637.7 [M + H]⁺), 100% purity Purification Method 50 |
| | | 3-fluoro-4- | |
| | | phenylbenzaldehyde | |
| | | CAS 57592-43-5 | |
| 1.137 | tert-Butyl (2-((8- carbamoylbenzo[c][2,6] naphthyridin-5- yl)amino)ethyl)(4-(((6- fluoro-1H-indol-2- yl)methyl)amino)butyl) carbamate | 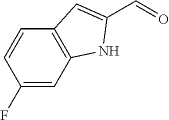 [SMILES: O=Cc1cc2ccc(F)cc2[nH]1] | LCMS (AM3): rt = 0.791 min, (600.3 [M + H]⁺), 99.4% purity. Purification Method 45 |
| | | 6-fluoro- | |
| | | 1H-indole-2- | |
| | | carbaldehyde | |
| | | CAS 933746-81-7 | |
| 1.142 | tert-Butyl (2-((8- carbamoylbenzo[c][2,6] naphthyridin-5- yl)amino)ethyl)(4-(((4- methyl-3-phenyl-1H- pyrazol-5- yl)methyl)amino)butyl) carbamate | 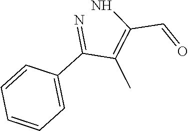 | LCMS (AM3): rt = 0.760 min, (623.6 [M + H]⁺), 98.4% purity. Purification Method 52 |
| | | 4-Methyl-3-phenyl-1H- | |
| | | pyrazole-5-carbaldehyde | |
| | | 1.141 | |
| 1.143 | tert-Butyl (2-((8- carbamoylbenzo[c][2,6] naphthyridin-5- yl)amino)ethyl)(4-(((3- phenyl-1H-pyrazol-5- yl)methyl)amino)butyl) carbamate | 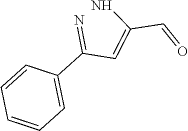 [SMILES: N=C(/C=C(\N)C=O)c1ccccc1] | LCMS (AM3): rt = 0.719 min, (609.2 [M + H]⁺), 95.2% purity Purification Method 52 |
| | | 3-phenyl-1H-pyrazole-5- | |
| | | carbaldehyde | |
| | | CAS 57204-65-6 | |
| 1.144 | tert-Butyl (2-((8- carbamoylbenzo[c][2,6] naphthyridin-5- yl)amino)ethyl)(4-(((6- (trifluoromethyl)quinolin- | 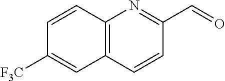 [SMILES: O=Cc1ccc2cc(C(F)(F)F)ccc2n1] | LCMS (AM3): rt = 0.806 min, (662.4 [M + H]⁺), 100% purity Purification Method 53 |
| | 2- | 6- | |
| | yl)methyl)amino)butyl) | (trifluoromethyl)quinoline- | |
| | carbamate | 2-carbaldehyde | |
| | | CAS 345895-60-5 | |
| 1.145 | tert-Butyl (2-((8- carbamoylbenzo[c][2,6] naphthyridin-5- yl)amino)ethyl)(4-(((5- methoxy-1H-indol-2- yl)methyl)amino)butyl) carbamate | 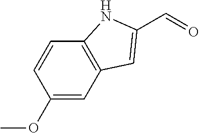 | LCMS (AM3): rt = 0.773 min, (612.3 [M + H]⁺), 99.8% purity. Purification Method 35 |
| | | 5-methoxy-1H-indole-2- | |
| | | carbaldehyde | |
| | | CAS 21778-81-4 | |
| 1.168 | tert-Butyl(2-((8- carbamoylbenzo[c][2,6] naphthyridin-5- yl)amino)ethyl)(4-(((6- chloro-1H-indol-2- yl)methyl)amino)butyl) carbamate | 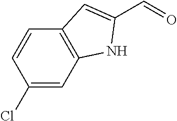 [SMILES: O=Cc1cc2ccc(Cl)cc2[nH]1] | LCMS (AM3): rt = 0.796 min, (616.3 [M + H]⁺), 99.7% purity. Purification Method 44 |
| | | 6-chloro-1H-indole-2- | |
| | | carbaldehyde | |
| | | CAS 53590-59-3 | |
| 1.185 | tert-Butyl (2-((8- carbamoylbenzo[c][2,6] naphthyridin-5- yl)amino)ethyl)(4-(((6- chloroquinolin-2- | 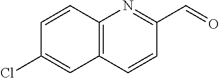 [SMILES: O=Cc1ccc2cc(Cl)ccc2n1] | LCMS (AM3): rt = 0.787 min, (628.3 [M + H]⁺), 100% purity. Purification Method 56 |
| | yl)methyl)amino)butyl) | 6- | |
| | carbamate | chloroquinoline-2- | |
| | | carbaldehyde | |
| | | CAS 59394-26-2 | |
| 1.205 | tert-Butyl (2-((8- carbamoylbenzo[c][2,6] naphthyridin-5- yl)amino)ethyl)(4-((3- chloro-4- | 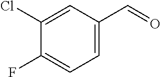 | LCMS (AM3): rt = 0.743 min, (595.5 [M + H]⁺), 100% purity Purification Method 51 |
| | fluorobenzyl)amino) | 3-chloro- | |
| | butyl)carbamate | 4-fluoro-benzaldehyde | |
| | | CAS 34328-61-5 | |
| 1.252 | tert-Butyl (2-((8- carbamoylbenzo[c][2,6] naphthyridin-5- yl)amino)ethyl)(4-((3- chlorobenzyl)amino) | 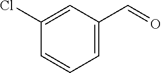 | LCMS (AM3): rt = 0.701 min, (577.1 [M + H]⁺), 58.6% purity. Purification Method 69 |
| | butyl)carbamate | 3-chlorobenzaldehyde | |
| | | CAS 587-04-2 | |
| 1.314 | tert-Butyl (2-((8- carbamoylbenzo[c][2,6] naphthyridin-5- yl)amino)ethyl)(4-(((2- methoxy-[1,1′- biphenyl]-4- yl)methyl)amino)butyl) carbamate | 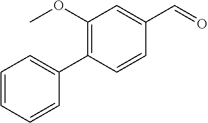 | LCMS (AM3): rt = 0.809 min, (649.5 [M + H]⁺), 100% purity Purification Method 50 |
| | | 2-Methoxy-[1,1′- | |
| | | biphenyl]-4-carbaldehyde | |
| | | CAS 248263-04-9 | |
| 1.317 | tert-Butyl (2-((8- carbamoylbenzo[c][2,6] naphthyridin-5- yl)amino)ethyl)(4-(((2- cyano-[1,1′-biphenyl]-4- yl)methyl)amino)butyl) carbamate | 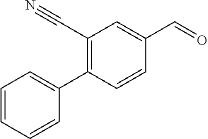 [SMILES: N#Cc1cc(C=O)ccc1-c1ccccc1] | LCMS (AM3): rt = 0.800 min, (644.4 [M + H]⁺), 66.7% purity Purification Method 72 |
| | | 4-Formyl-[1,1′-biphenyl]-2- | |
| | | carbonitrile | |
| | | CAS 25331-69-5 | |
| 1.327 | tert-Butyl (2-((8- carbamoylbenzo[c][2,6] naphthyridin-5- yl)amino)ethyl)(4-((3- cyano-4- cyclobutoxybenzyl) amino)butyl)carbamate | 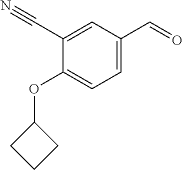 [SMILES: N#Cc1cc(C=O)ccc1OC1CCC1] | LCMS (AM3): rt = 0.729 min, (638.2 [M + H]⁺), 37.5% purity Purification Method 73 |
| | | 2- | |
| | | cyclobutoxy-5- | |
| | | formylbenzonitrile (Ref: | |
| | | WO2022185041) | |
| 1.370 | tert-Butyl (2-((8- carbamoylbenzo[c][2,6] naphthyridin-5- yl)amino)ethyl)(4-((3- cyano-4- (trifluoromethoxy)benzyl) amino)butyl)carbamate | 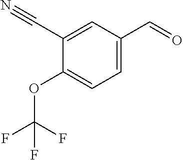 [SMILES: N#Cc1cc(C=O)ccc1OC(F)(F)F] | LCMS (AM3): rt = 0.601 min, (652.1 [M + H]⁺), 55.9% purity Purification Method 78 |
| | | 5- | |
| | | Formyl-2- | |
| | | (trifluoromethoxy)benzo- | |
| | | nitrile (Ref: | |
| | | WO2022185041) | |

### Synthesis of Intermediate H

### tert-butyl ((2-chloro-[1,1′-biphenyl]-4-yl)methyl)(3-((3,3-diethoxypropyl)amino)-3-oxopropyl)carbamate, 1.77

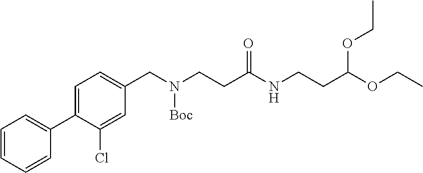 [SMILES: CCOC(CCNC(=O)CCN(Cc1ccc(-c2ccccc2)c(Cl)c1)C(=O)OC(C)(C)C)OCC]

To a solution of Intermediate D (10 g, 25.65 mmol), 3,3-diethoxypropan-1-amine (4.53 g, 30.78 mmol) and TEA (10.71 mL, 76.95 mmol) in THF (100 mL) was added T₃P (24.48 g, 38.47 mmol, 50% in EA) at 0° C. The resulting mixture was stirred at 0° C. for 2 h. The mixture was diluted with H₂O (500 mL) and extracted with EA (200 mL×2). The combined organic phases were washed (brine, 200 mL), dried (Na₂SO₄) and concentrated in vacuo. The residue was purified (PM5) to give compound 1.77 (11.2 g, 21.58 mmol, 84.1% yield) as a colourless oil.

LCMS (AM3): rt=1.057 min, (541.2 [M+Na]⁺), 76% purity.

### tert-butyl ((2-chloro-[1,1′-biphenyl]-4-yl)methyl)(3-oxo-3-((3-oxopropyl)amino)propyl)carbamate, Intermediate H

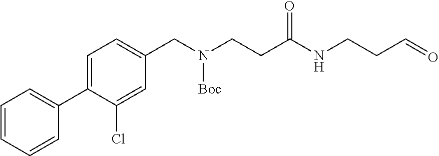 [SMILES: CC(C)(C)OC(=O)N(CCC(=O)NCCC=O)Cc1ccc(-c2ccccc2)c(Cl)c1]

A solution of compound 1.77 (1 g, 1.93 mmol) in AcOH (5 mL) and H₂O (5 mL) was stirred at 30° C. for 2. The mixture was basified with saturated aq. NaHCO₃ solution to pH=8 and then extracted with EA (50 mL×2). The combined organic phases were washed (brine, 50 mL), dried (Na₂SO₄) and concentrated in vacuo to give Intermediate H (850 mg, 1.91 mmol, 99.1% yield) as a colourless oil, which was used directly without further purification.

LCMS (AM3): rt=0.961 min, (467.2 [M+Na]⁺), 86.8% purity.

### Synthesis of Intermediate 1.83

### Methyl 5-(4-{bis[(tert-butoxy)carbonyl]amino}butyl)benzo[c]2,6-naphthyridine-8-carboxylate 1.81

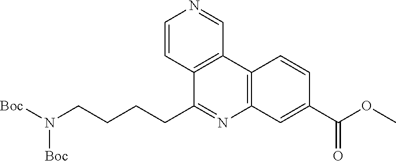 [SMILES: COC(=O)c1ccc2c(c1)nc(CCCCN(C(=O)OC(C)(C)C)C(=O)OC(C)(C)C)c1ccncc12]

To a solution of tert-butyl N-(but-3-en-1-yl)-N-[(tert-butoxy) carbonyl]carbamate (600 mg, 2.21 mmol) (WO2002081467) in anhydrous THF (6 mL) was added a solution of 9-BBN (0.5 M, 4.86 mL) in THE dropwise under N₂ at 20° C. The resulting reaction mixture was stirred at 20° C. for 16 h. Consequently, tert-butyl N-[4-(9-borabicyclo[3.3.1]nonan-9-yl)butyl]-N-tert-butoxycarbonyl-carbamate was obtained as a solution in THF (approximately 0.204 M), which was added to a solution of compound 1.1 (170 mg, 623.43 μmol), Pd(dppf)Cl₂·CH₂Cl₂ (50.91 mg, 62.34 μmol) and K₃PO₄ (397.00 mg, 1.87 mmol) in anhydrous DMF (10 mL) under N₂. The resulting reaction mixture was stirred at 85° C. for 18 h. The reaction mixture was diluted with H₂O (30 mL) and extracted with EA (20 mL×2). The combined organic layers were washed (brine, 10 mL), dried (Na₂SO₄) and filtered. The filtrate was concentrated in vacuo and the residue was purified (PM19) to afford compound 1.81 (60 mg, 117.74 μmol, 18.9% yield) as a yellow solid.

LCMS (AM3): rt=1.101 min, (532.5 [M+Na]⁺).

### Methyl 5-(4-aminobutyl)benzo[c]2,6-naphthyridine-8-carboxylate 1.82

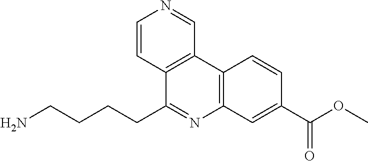 [SMILES: COC(=O)c1ccc2c(c1)nc(CCCCN)c1ccncc12]

To a solution of compound 1.81 (60 mg, 118 μmol) in EA (4 mL) was added a solution of HCl in EA (4 M, 2.82 mL) and the mixture was stirred at 25° C. for 1 h. The reaction mixture was concentrated in vacuo to afford compound 1.82 (40 mg, HCl salt) as a yellow solid, which was used without purification.

LCMS (AM3): rt=0.690 min, (310.2 [M+H]⁺).

### Methyl 5-[4-({4-[({2-chloro-[1,1′-biphenyl]-4-yl}methyl)amino]butyl}amino)butyl]benzo[c]2,6-naphthyridine-8-carboxylate 1.83

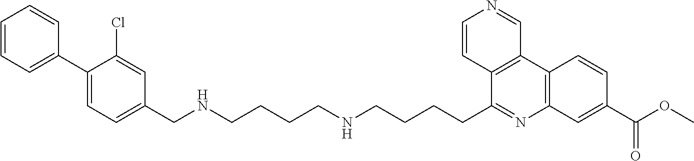 [SMILES: COC(=O)c1ccc2c(c1)nc(CCCCNCCCCNCc1ccc(-c3ccccc3)c(Cl)c1)c1ccncc12]

To a solution of compound 1.7 (50.2 mg, 129 μmol) and compound 1.82 (40 mg, 129 μmol) in MeOH (1 mL) was added NaBH₃CN (16.3 mg, 259 μmol) at 20° C. and the mixture was stirred at 20° C. for 1 h. The mixture was diluted with EA (5 mL), washed with saturated aq. NaHCO₃ solution (3×5 mL), dried (Na₂SO₄), filtered and concentrated in vacuo. The residue was purified (PM48) to afford compound 1.83 (40 mg, 61.3 μmol, 47.4% yield) as a yellow solid.

LCMS (AM3): rt=0.818 min, (581.5 [M+H]⁺), 89% purity.

### Synthesis of Intermediate 1.85

### 5-[(2-aminoethyl)amino]benzo[c]2,6-naphthyridine-8-carboxylic acid 1.84

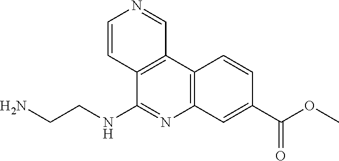 [SMILES: COC(=O)c1ccc2c(c1)nc(NCCN)c1ccncc12]

A mixture of Intermediate A (300 mg, 1.01 mmol) and LiOH·H₂O (212 mg, 5.06 mmol) in MeOH (5 mL) was stirred at 25° C. for 1 h under N₂. The reaction mixture was adjusted to pH 6 with aq. HCl (1 M), then diluted with H₂O (10 mL) and extracted with EA (3×10 mL). The combined organic layers were washed with H₂O (2×10 mL) and brine (10 mL), dried (Na₂SO₄) and filtered. The filtrate was concentrated in vacuo to give compound 1.84 (200 mg, 708 μmol, 70.0% yield) as a dark brown oil, which was used directly without further purification.

### 5-({2-[(4-{[(tert-butoxy)carbonyl]({2-chloro-[1,1′-biphenyl]-4-yl}methyl)amino}butyl)amino]ethyl}amino)benzo[c]2,6-naphthyridine-8-carboxylic acid 1.85

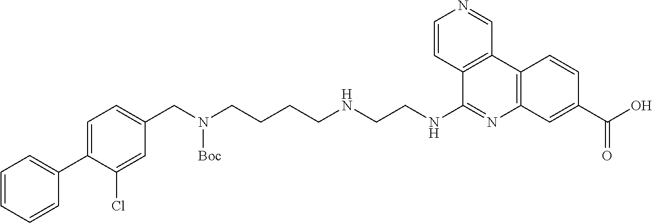 [SMILES: CC(C)(C)OC(=O)N(CCCCNCCNc1nc2cc(C(=O)O)ccc2c2cnccc12)Cc1ccc(-c2ccccc2)c(Cl)c1]

A mixture of compound 1.7 (200 mg, 516 μmol), compound 1.84 (140 mg, 496 μmol) and NaBH₃CN (48 mg, 764 μmol) in MeOH (2 mL) was stirred at 70° C. for 2 h The reaction mixture was diluted with H₂O (10 mL) and extracted with EA (3×10 mL). The combined organic layers were washed with H₂O (2×10 mL) and brine (10 mL), dried (Na₂SO₄) and filtered. The filtrate was concentrated in vacuo to give compound 1.85 (200 mg, 306 μmol, 59.3% yield) as a dark brown solid, which was used directly without further purification.

### Synthesis of Intermediate 1.87

### Methyl 5-[(3-aminopropyl)amino]benzo[c]2,6-naphthyridine-8-carboxylate 1.86

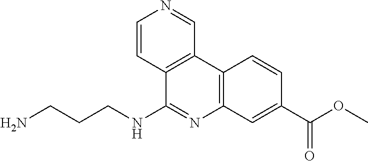 [SMILES: COC(=O)c1ccc2c(c1)nc(NCCCN)c1ccncc12]

A solution of compound 1.26 (280 mg, 682 μmol) in dioxane (15 mL) was treated with a solution of HCl in dioxane (4 M, 15 mL) and stirred at 25° C. for 1 h under N₂. The reaction mixture was concentrated in vacuo to give compound 1.86 (300 mg, HCl salt) as a black solid.

¹H NMR (400 MHz, DMSO-d₆) δ: 10.18 (s, 1H), 9.05 (d, J=5.6 Hz, 1H), 8.88 (d, J=8.4 Hz, 2H), 8.20-8.13 (m, 3H), 8.01 (d, J=7.9 Hz, 1H), 3.94 (s, 3H), 3.51-3.48 (m, 2H), 3.42 (d, J=5.1 Hz, 2H), 2.15-2.07 (m, 2H) ppm.

### Methyl 5-({3-[(4-{[(tert-butoxy)carbonyl]({2-chloro-[1,1′-biphenyl]-4-yl}methyl)amino}butyl)amino]propyl}amino)benzo[c]2,6-naphthyridine-8-carboxylate 1.8

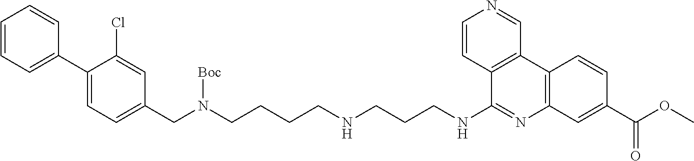 [SMILES: COC(=O)c1ccc2c(c1)nc(NCCCNCCCCN(Cc1ccc(-c3ccccc3)c(Cl)c1)C(=O)OC(C)(C)C)c1ccncc12]

A mixture of compound 1.7 (100 mg, 258 μmol), compound 1.86 (80.0 mg, 258 μmol) and NaBH₃CN (25.0 mg, 398 μmol) in MeOH (2 mL) was stirred at 70° C. for 2 h. The reaction mixture was used directly in the next step without work-up.

### 5-({3-[(4-{[(tert-butoxy)carbonyl]({2-chloro-[1,1′-biphenyl]-4-yl}methyl)amino}butyl)amino]propyl}amino)benzo[c]2,6-naphthyridine-8-carboxylic acid 1.87

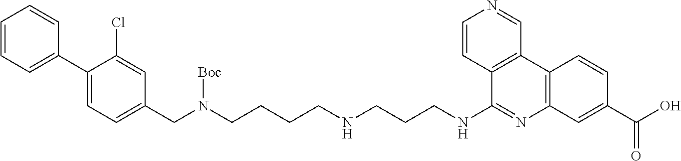 [SMILES: CC(C)(C)OC(=O)N(CCCCNCCCNc1nc2cc(C(=O)O)ccc2c2cnccc12)Cc1ccc(-c2ccccc2)c(Cl)c1]

The reaction mixture containing compound 1.8 (175 mg, 258 μmol) was treated with LiOH·H₂O (54.0 mg, 1.29 mmol) in MeOH (3 mL) and stirred at 70° C. for 1 h. The reaction mixture was adjusted to pH 5 with aq. HCl (1 M), then diluted with H₂O (10 mL) and extracted with EA (3×10 mL). The combined organic layers were washed with H₂O (2×10 mL) and brine (10 mL), dried (Na₂SO₄), filtered and concentrated in vacuo to give compound 1.87 (150 mg, 224 μmol, 87.5% yield) as a yellow solid, which was used directly without further purification.

### Synthesis of Intermediate 1.95

### Methyl 5-((3-((tert-butoxycarbonyl)amino)-3-methylbutyl)amino)benzo[c][2,6]naphthyridine-8-carboxylate 1.91

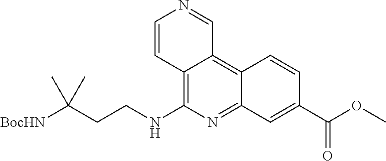 [SMILES: COC(=O)c1ccc2c(c1)nc(NCCC(C)(C)NC(=O)OC(C)(C)C)c1ccncc12]

To a solution of compound 1.1 (250 mg, 916.81 μmol) in DMSO (10 mL) was added DIPEA (236.98 mg, 1.83 mmol) and tert-butyl N-(3-amino-1,1-dimethyl-propyl) carbamate (229.84 mg, 962.65 μmol, HCl salt) at RT. The mixture was heated to 80° C. and stirred for 12 h. The mixture was poured into H₂O and extracted with EA (50 mL×2). The combined organic phases were washed (brine, 50 mL), dried (Na₂SO₄) and concentrated in vacuo. The residue was purified (PM38) to afford compound 1.91 (300 mg, 542.95 μmol, 53.8% yield, TFA salt) as a yellow solid.

LCMS (AM3): rt=0.826 min, (439.8 [M+H]⁺), 59.6% purity

### 5-((3-((tert-Butoxycarbonyl)amino)-3-methylbutyl)amino)benzo[c][2,6]naphthyridine-8-carboxylic acid 1.92

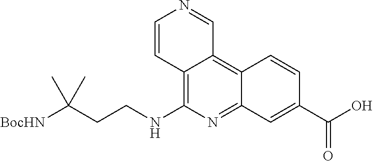 [SMILES: CC(C)(CCNc1nc2cc(C(=O)O)ccc2c2cnccc12)NC(=O)OC(C)(C)C]

To a solution of compound 1.91 (280 mg, 506.75 μmol) in THF (15 mL), MeOH (1.5 mL) and H₂O (1.5 mL) was added LiOH·H₂O (425.30 mg, 10.13 mmol) at 20° C. The mixture was stirred for 12 h. The organic solvent was removed under vacuum and the aq. phase was acidified with 1 N HCl to PH=5. The mixture was extracted with EA (50 mL×2). The combined organic phase was dried (Na₂SO₄), filtered and concentrated in vacuo to afford compound 1.92 (280 mg), which was used directly without further purification.

LCMS (AM3): rt=0.789 min, (425.2 [M+H]⁺), 79.27% purity.

### tert-Butyl (4-((8-carbamoylbenzo[c][2,6]naphthyridin-5-yl)amino)-2-methylbutan-2-yl)carbamate 1.93

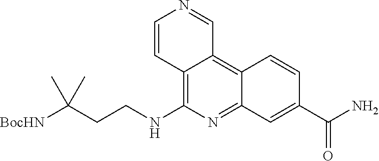 [SMILES: CC(C)(CCNc1nc2cc(C(N)=O)ccc2c2cnccc12)NC(=O)OC(C)(C)C]

To a solution of compound 1.92 (280 mg, 659.61 μmol) in THF (10 mL) was added HATU (300.97 mg, 791.53 μmol), DIPEA (170.50 mg, 1.32 mmol) and NH₄Cl (38.81 mg, 725.57 μmol), sequentially at 25° C. The mixture was stirred at 25° C. for 12 h. The mixture was concentrated and purified (PM39) to afford compound 1.93 (120 mg, 283.35 μmol, 43% yield) as a yellow solid.

LCMS (AM3): rt=0.763 min, (424.1 [M+H]⁺), 99.6% purity.

### 5-((3-Amino-3-methylbutyl)amino)benzo[c][2,6]naphthyridine-8-carboxamide 1.94

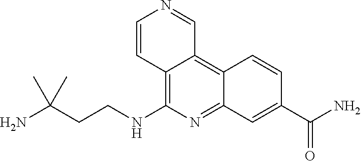 [SMILES: CC(C)(N)CCNc1nc2cc(C(N)=O)ccc2c2cnccc12]

To a solution of compound 1.93 (120 mg, 283.35 μmol) in DCM (2 mL) was added TFA (770.00 mg, 6.75 mmol) at 25° C. The mixture was stirred for 1 h. The reaction was concentrated in vacuo to afford compound 1.94 (120 mg, TFA salt), which was used directly without purification.

LCMS (AM3): rt=0.511 min, (324.2 [M+H]⁺), 93.4% purity.

### tert-Butyl (3-((4-((8-carbamoylbenzo[c][2,6]naphthyridin-5-yl)amino)-2-methylbutan-2-yl)amino)-3-oxopropyl)((2-chloro-[1,1′-biphenyl]-4-yl)methyl)carbamate 1.95

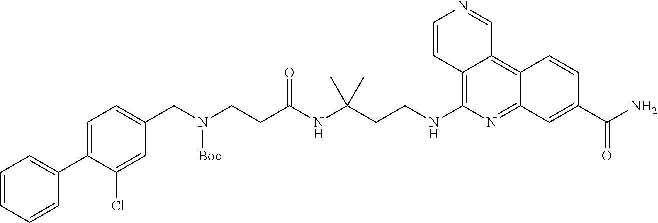 [SMILES: CC(C)(CCNc1nc2cc(C(N)=O)ccc2c2cnccc12)NC(=O)CCN(Cc1ccc(-c2ccccc2)c(Cl)c1)C(=O)OC(C)(C)C]

To a solution of Intermediate D (87.0 mg, 223.15 μmol) in THF (10 mL) was added DIPEA (57.70 mg, 446.46 μmol) and HATU (101.86 mg, 267.89 μmol) at 25° C. After stirring for 30 min, compound 1.94 (94.5 mg, 292.22 μmol) was added. The mixture was stirred at 25° C. for 11.5 h. The mixture was concentrated in vacuo and purified (PM40) to afford compound 1.95 (120 mg, 148.28 μmol, 50.7% yield, TFA salt) as a yellow solid.

LCMS (AM3): rt=0.844 min, (695.4 [M+H]⁺), 77.6% purity.

### Synthesis of Intermediate 1.100

### 3-Chloro-4-cyclopentylbenzaldehyde 1.100

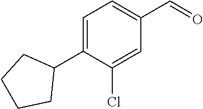 [SMILES: O=Cc1ccc(C2CCCC2)c(Cl)c1]

To a solution of 3-Chloro-4-cyclopentylphenylmethanol (CAS 1228582-57-7, 220 mg, 1.04 mmol) in DCM (20 mL) was added manganese (IV) oxide (907.74 mg, 10.44 mmol) at 30° C. The reaction mixture was stirred at 30° C. for 12 h. The mixture was filtered and the filtrate was concentrated in vacuo. The crude product was purified (PM13) to afford compound 1.100 (150 mg, 718.79 μmol, 62.6% yield) as a colourless oil.

¹H NMR (400 MHz, CHCl₃-d) δ: 9.94 (s, 1H), 7.86 (d, J=1.5 Hz, 1H), 7.73 (dd, J=1.6, 7.9 Hz, 1H), 7.48 (d, J=7.8 Hz, 1H), 3.56-3.47 (m, 1H), 2.20-2.10 (m, 2H), 1.90-1.70 (m, 4H), 1.65-1.55 (m, 2H) ppm.

### Synthesis of Intermediate 1.105

### tert-Butyl (2-((8-carbamoylbenzo[c][2,6]naphthyridin-5-yl)amino)ethyl) (4-(((2-chloro-2′-(cyanomethyl)-[1,1′-biphenyl]-4-yl)methyl)amino)butyl)carbamate 1.105

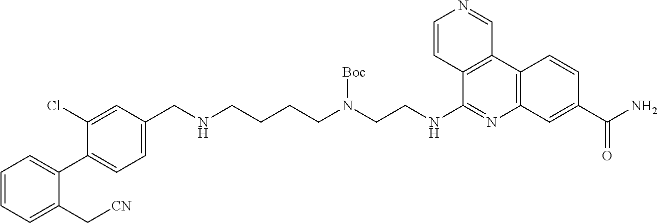 [SMILES: CC(C)(C)OC(=O)N(CCCCNCc1ccc(-c2ccccc2CC#N)c(Cl)c1)CCNc1nc2cc(C(N)=O)ccc2c2cnccc12]

To a mixture of compound 1.104 (100 mg, 0.135 mmol, TFA salt) in 1,4-dioxane (8 mL) and H₂O (2 mL) was added K₂CO₃ (80 mg, 0.579 mmol) followed by 2-(2-bromophenyl) acetonitrile (0.035 mL, 0.27 mmol). The reaction mixture was degassed and purged with N₂ (×3), then Pd(dppf)Cl₂ (10 mg, 0.014 mmol) was added. The reaction was heated to 80° C. and stirred for 16 h under N₂. The reaction mixture was concentrated in vacuo and the residue was purified (PM40) to afford compound 1.105 (27 mg, 23% yield, TFA salt) as a yellow solid.

LCMS (AM5): rt=0.666 min, (692.2 [M+H]⁺), 92.9% purity.

### Synthesis of Intermediate 1.108

### (2-((2-(2-Bromophenoxy)ethoxy)methoxy)ethyl)trimethylsilane 1.107

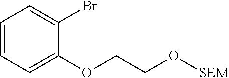 [SMILES: C[Si](C)(C)CCOCOCCOc1ccccc1Br]

A mixture of 2-(2-bromophenoxy) ethanol (230 mg, 1.06 mmol), DIPEA (0.46 mL, 2.65 mmol) and (2-(chloromethoxy)ethyl)trimethylsilane (0.28 mL, 1.59 mmol) in DCM (10 mL) was stirred at RT for 20 h. The reaction mixture was concentrated in vacuo and purified (PM14) to afford compound 1.107 (170 mg, 46.2% yield) as a colourless oil.

¹H NMR (400 MHz, MeOH-d₄) δ: 7.52 (dd, J=8.0 Hz, 1.6 Hz, 1H), 7.31-7.27 (m, 1H), 7.04 (dd, J=8.4 Hz, 1.2 Hz, 1H), 6.87-6.83 (m, 1H), 4.77 (s, 2H), 4.21-4.19 (m, 2H), 3.94-3.91 (m, 2H), 3.71-3.67 (m, 2H), 0.93 (t, J=8.0 Hz, 2H), 0.01 (s, 9H) ppm.

### 2-Chloro-2′-(2-((2-(trimethylsilyl)ethoxy)methoxy)ethoxy)-[1,1′-biphenyl]-4-carbaldehyde 1.108

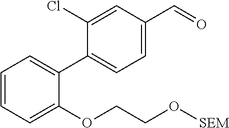 [SMILES: C[Si](C)(C)CCOCOCCOc1ccccc1-c1ccc(C=O)cc1Cl]

A mixture of compound 1.107 (170 mg, 0.489 mmol), (2-chloro-4-formylphenyl) boronic acid (180 mg, 0.976 mmol) and K₂CO₃ (170 mg, 1.23 mmol) in 1,4-dioxane (8 mL) and H₂O (2 mL) was degassed and purged with N₂ (×3), then Pd(dppf)Cl₂ (36 mg, 0.049 mmol) was added. The reaction was degassed and purged with N₂ (×3). The reaction mixture was heated to 80° C. and stirred for 18 h. The reaction mixture was filtered and the filtrate was concentrated in vacuo. The crude product was purified by prep-TLC (PM8) to afford compound 1.108 (90 mg, crude) as a light brown oil, which was used directly.

### Synthesis of Intermediate 1.117

### Methyl 5-((3-(3-aminopropoxy)propyl)amino)benzo[c][2,6]naphthyridine-8-carboxylate 1.113

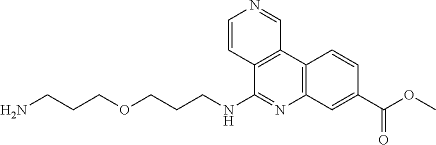 [SMILES: COC(=O)c1ccc2c(c1)nc(NCCCOCCCN)c1ccncc12]

A mixture of 3,3′-oxybis(propan-1-amine) (1.37 g, 10.34 mmol), compound 1.1 (600 mg, 2.07 mmol) and DIPEA (534.63 mg, 4.14 mmol) in DMSO (6 mL) was heated to 65° C. and stirred for 16 h. The mixture was poured into H₂O and extracted with EA (50 mL×2). The combined organic phases were washed (brine, 50 mL), dried (Na₂SO₄), filtered and concentrated in vacuo. The residue was purified (PM46) to afford compound 1.113 (500 mg, 1.29 mmol, 62.3% yield) as a brown oil.

LCMS (AM3): rt=0.686 min, (369.1 [M+H]⁺), 95.4% purity.

### Methyl 5-((3-(3-(((2-chloro-[1,1′-biphenyl]-4-yl)methyl)amino)propoxy)propyl)amino)benzo[c][2,6]naphthyridine-8-carboxylate 1.114

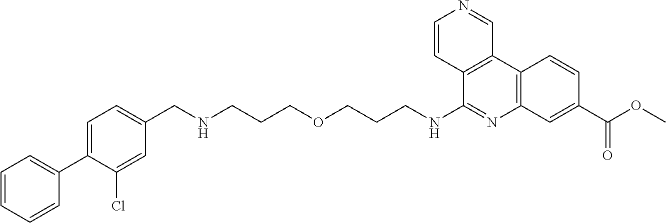 [SMILES: COC(=O)c1ccc2c(c1)nc(NCCCOCCCNCc1ccc(-c3ccccc3)c(Cl)c1)c1ccncc12]

A mixture of compound 1.113 (440 mg, 1.13 mmol) and 3-chloro-4-phenylbenzaldehyde (CAS 57592-44-6, 220 mg, 1.02 mmol) in MeOH (2 mL) was stirred at 25° C. for 3 h, then NaBH₃CN (127.62 mg, 2.03 mmol) was added. The mixture was stirred at 25° C. for 12 h. The mixture was concentrated in vacuo and purified (PM40) to afford compound 1.114 (120 mg, 0.167 mmol, 16.4% yield, TFA salt) as a yellow solid.

LCMS (AM3): rt=0.813 min, (569.2 [M+H]⁺), 94.5% purity.

### Methyl 5-((3-(3-((tert-butoxycarbonyl) ((2-chloro-[1,1′-biphenyl]-4-yl)methyl)amino)propoxy)propyl)amino)benzo[c][2,6]naphthyridine-8-carboxylate 1.115

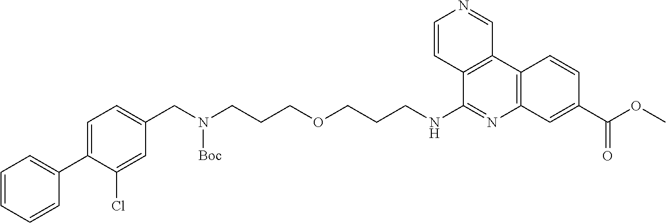 [SMILES: COC(=O)c1ccc2c(c1)nc(NCCCOCCCN(Cc1ccc(-c3ccccc3)c(Cl)c1)C(=O)OC(C)(C)C)c1ccncc12]

To a mixture of compound 1.114 (170 mg, 0.299 mmol, TFA salt) and TEA (90.68 mg, 0.896 mmol) in DCM (10 mL) was added Boc₂O (78.24 mg, 0.358 mmol) at 25° C. The mixture was stirred at 25° C. for 1 h. The mixture was diluted with H₂O (30 mL) and extracted with DCM (30 mL×2). The combined organic phases were washed (brine, 50 mL), dried (Na₂SO₄), filtered and concentrated in vacuo to afford compound 1.115 (130 mg, 0.194 mmol, 65% yield) as a yellow oil, which was used directly without further purification.

LCMS (AM3): rt=0.942 min, (669.3 [M+H]⁺), 89.5% purity.

### 5-((3-(3-((tert-Butoxycarbonyl)((2-chloro-[1,1′-biphenyl]-4-yl)methyl)amino)propoxy)propyl)amino)benzo[c][2,6]naphthyridine-8-carboxylic acid 1.116

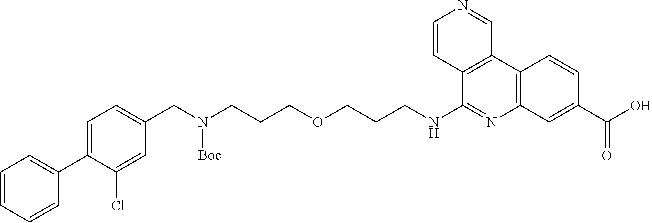 [SMILES: CC(C)(C)OC(=O)N(CCCOCCCNc1nc2cc(C(=O)O)ccc2c2cnccc12)Cc1ccc(-c2ccccc2)c(Cl)c1]

To a mixture of compound 1.115 (130 mg, 0.194 mmol) in THF (9 mL), MeOH (1 mL) and H₂O (1 mL) was added LiOH·H₂O (81.52 mg, 1.94 mmol) at 25° C. The mixture was stirred at 25° C. for 20 h. The organic solvent was removed in vacuo. The remaining aq. phase was acidified with HCl (1 N, 5 mL) and extracted with EA (15 mL×2). The combined organic phases were washed (brine, 20 mL), dried (Na₂SO₄), filtered and concentrated in vacuo to afford compound 1.116 (130 mg) as a yellow oil which was used directly without further purification.

LCMS (AM3): rt=0.865 min, (655.3 [M+H]⁺), 93.4% purity.

### tert-Butyl (3-(3-((8-carbamoylbenzo[c][2,6]naphthyridin-5-yl)amino)propoxy)propyl)((2-chloro-[1,1′-biphenyl]-4-yl)methyl)carbamate 1.117

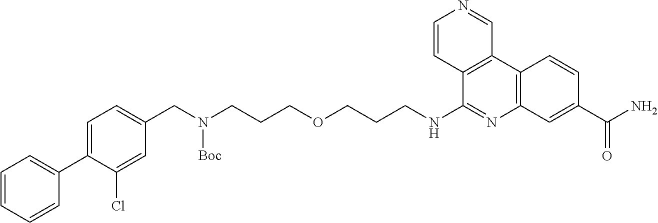 [SMILES: CC(C)(C)OC(=O)N(CCCOCCCNc1nc2cc(C(N)=O)ccc2c2cnccc12)Cc1ccc(-c2ccccc2)c(Cl)c1]

To a mixture of compound 1.116 (120 mg, 0.183 mmol) in THF (5 mL) was added HATU (90 mg, 0.237 mmol), DIPEA (74.20 mg, 0.574 mmol) and NH₄Cl (50 mg, 0.935 mmol), sequentially at 25° C. The mixture was stirred at 25° C. for 14 h. The mixture was poured into H₂O (20 mL) and the aq. phase was extracted with EA (20 mL×2). The combined organic phases were washed (brine, 30 mL), dried (Na₂SO₄), filtered and concentrated in vacuo to afford compound 1.117 (130 mg) as a yellow oil which was used directly without further purification.

LCMS (AM3): rt=0.865 min, (654.3 [M+H]⁺), 85% purity.

### Synthesis of Intermediate 1.118

### 5-((2-(3-Chloropropylsulfonamido)ethyl)amino)benzo[c][2,6]naphthyridine-8-carboxamide 1.118

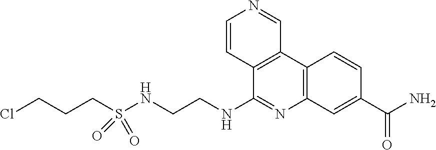 [SMILES: NC(=O)c1ccc2c(c1)nc(NCCNS(=O)(=O)CCCCl)c1ccncc12]

To a mixture of Intermediate F (250 mg, 0.787 mmol, HCl salt) and TEA (239 mg, 2.36 mmol) in DMF (10 mL) was added 3-chloropropane-1-sulfonyl chloride (208.93 mg, 1.18 mmol) at 0° C. The mixture was warmed to 25° C. and stirred for 2 h. The mixture was concentrated in vacuo and purified (PM39) to afford compound 1.118 (150 mg, 0.356 mmol, 45.2% yield) as a yellow solid.

LCMS (AM3): rt=0.616 min, (422.2 [M+H]⁺), 100% purity.

### Synthesis of Intermediate 1.141

### Ethyl 4-methyl-3-phenyl-1H-pyrazole-5-carboxylate 1.139

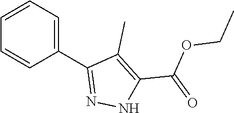 [SMILES: CCOC(=O)c1[nH]nc(-c2ccccc2)c1C]

To a mixture of compound 1.138 (ref: WO2015095767, 400 mg, 1.43 mmol) and phenylboronic acid (261.22 mg, 2.14 mmol) in 1,4-dioxane (5 mL) and H₂O (0.5 mL) was added Pd(dppf)Cl₂·CH₂Cl₂ (116.64 mg, 142.83 μmol) and sodium carbonate (378.45 mg, 3.57 mmol) under N₂ at RT. The mixture was stirred at 85° C. for 12 h. The mixture was concentrated in vacuum and purified (PM40) to afford compound 1.139 (300 mg, 1.19 mmol, 83% yield) as a brown gum.

LCMS (AM3): rt=0.906 min, (231.2 [M+H]⁺), 91.1% purity.

### (4-Methyl-3-phenyl-1H-pyrazol-5-yl)methanol 1.140

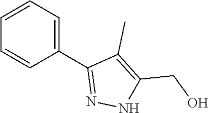

To a mixture of compound 1.139 (300 mg, 1.19 mmol) in THF (10 mL) was added LAH (44.99 mg, 1.19 mmol) in portions at 0° C. under N₂. The mixture was then warmed to 25° C. and stirred for 5 h. The mixture was cooled to 0° C., diluted with EA (10 mL) and quenched with H₂O (0.2 mL) followed by addition of aq. 10% NaOH (0.2 mL) and H₂O (0.6 mL). The resulting suspension was dried (Na₂SO₄), filtered and concentrated in vacuo to afford compound 1.140 (300 mg) as a brown gum, which was used directly without purification.

LCMS (AM3): rt=0.747 min, (171.1 [M−H₂O+H]⁺), 59.5% purity.

### 4-Methyl-3-phenyl-1H-pyrazole-5-carbaldehyde 1.141

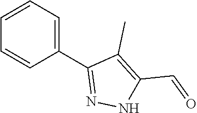

To a mixture of compound 1.140 (300 mg, 1.59 mmol) in DCM (10 mL) was added manganese (IV) oxide (1.39 g, 15.94 mmol) at 25° C. The mixture was stirred at 25° C. for 12 h. The mixture was concentrated in vacuo and purified (PM24) afford compound 1.141 (100 mg, 520.92 μmol, 32.7% yield) as a white solid.

LCMS (AM3): rt=0.845 min, (187.2 [M+H]⁺), 96.7% purity.

### Synthesis of Intermediate I

### tert-Butyl 3-chloro-4-(trifluoromethoxy)benzyl(4-oxobutyl)carbamate, Intermediate I

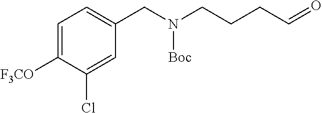 [SMILES: CC(C)(C)OC(=O)N(CCCC=O)Cc1ccc(OC(F)(F)F)c(Cl)c1]

To a solution of tert-Butyl 3-chloro-4-(trifluoromethoxy)benzyl (4-hydroxybutyl)carbamate (Ref: WO2022185041, 1.45 g, 3.64 mmol) in DCM (10 mL) was added DMP (1.86 g, 4.37 mmol) at 20° C. The resulting mixture was stirred at 20° C. for 1 h. The mixture was concentrated in vacuo and purified (PM8) to afford Intermediate I (1.15 g, 2.91 mmol, 79.7% yield) as a light yellow oil.

LCMS (AM3): rt=1.036 min, (418.1 [M+Na]⁺), 72.2% purity.

### Synthesis of Intermediate 1.163

### Methyl 5-((2-((tert-butoxycarbonyl)(methyl)amino)ethyl)amino)benzo[c][2,6]naphthyridine-8-carboxylate 1.159

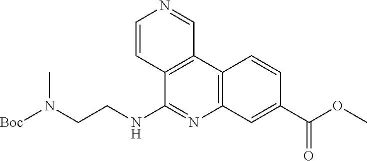 [SMILES: COC(=O)c1ccc2c(c1)nc(NCCN(C)C(=O)OC(C)(C)C)c1ccncc12]

A mixture of compound 1.1 (500 mg, 1.83 mmol), tert-butyl (2-aminoethyl)(methyl)carbamate (383.39 mg, 2.20 mmol) and DIPEA (710.93 mg, 5.50 mmol) in DMSO (10 mL) was stirred at 80° C. for 15 h. The mixture was poured into H₂O (100 mL) and extracted with EA (100 mL×2). The combined organic phases were washed (brine, 50 mL), dried (Na₂SO₄) and concentrated in vacuo to afford compound 1.159 (600 mg) as an off-white solid, which was used directly without further purification.

LCMS (AM3): rt=0.818 min, (411.2 [M+H]⁺), 83.7% purity.

### 5-((2-((tert-Butoxycarbonyl)(methyl)amino)ethyl)amino)benzo[c][2,6]naphthyridine-8-carboxylic acid 1.160

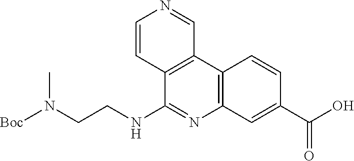 [SMILES: CN(CCNc1nc2cc(C(=O)O)ccc2c2cnccc12)C(=O)OC(C)(C)C]

To a mixture of compound 1.159 (600 mg, 1.46 mmol) in THF (10 mL), MeOH (10 mL) and H₂O (10 mL) was added LiOH·H₂O (184.02 mg, 4.39 mmol) at 25° C. The resulting mixture was stirred at 25° C. for 3 h. The solvent was removed under reduced pressure and the residue was acidified with aq. HCl (1 N) to pH4. The resulting solid was collected by filtration and dried under vacuum to afford compound 1.160 (350 mg) as a yellow solid, which was used directly without further purification.

LCMS (AM3): rt=0.761 min, (397.1 [M+H]⁺), 90% purity.

### tert-Butyl(2-((8-carbamoylbenzo[c][2,6]naphthyridin-5-yl)amino)ethyl)(methyl)carbamate 1.161

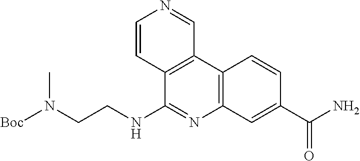 [SMILES: CN(CCNc1nc2cc(C(N)=O)ccc2c2cnccc12)C(=O)OC(C)(C)C]

To a solution of compound 1.160 (350 mg, 882.86 μmol) in DMF (10 mL) was added HOBt (180 mg, 1.33 mmol), EDCI (250 mg, 1.30 mmol), DIPEA (170 mg, 1.32 mmol) and NH₄Cl (150 mg, 2.80 mmol) at 20° C. The resulting mixture was stirred at 20° C. for 15 h. The reaction mixture was poured into H₂O (50 mL), and the resulting solid collected by filtration and dried under vacuum to afford compound 1.161 (230 mg) as a yellow solid, which was used directly without further purification.

LCMS (AM3): rt=0.730 min, (396.2 [M+H]⁺), 73.8% purity.

### 5-((2-(Methylamino)ethyl)amino)benzo[c][2,6]naphthyridine-8-carboxamide 1.162

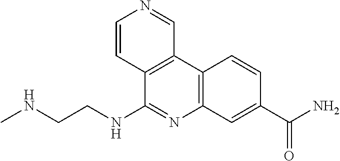 [SMILES: CNCCNc1nc2cc(C(N)=O)ccc2c2cnccc12]

To a solution of compound 1.161 (230 mg, 581.61 μmol) in 1,4-dioxane (5 mL) was added a solution of HCl in 1,4-dioxane (4 M, 5 mL) at 20° C. The resulting mixture was stirred at 20° C. for 15 h. The mixture was concentrated in vacuo to afford compound 1.162 (200 mg, HCl salt) as a yellow solid, which was used directly without further purification.

LCMS (AM3): rt=0.172 min, (296.1 [M+H]⁺), 94.9% purity.

### tert-Butyl (4-((2-((8-carbamoylbenzo[c][2,6]naphthyridin-5-yl)amino)ethyl)(methyl)amino)butyl)(3-chloro-4-(trifluoromethoxy)benzyl)carbamate 1.163

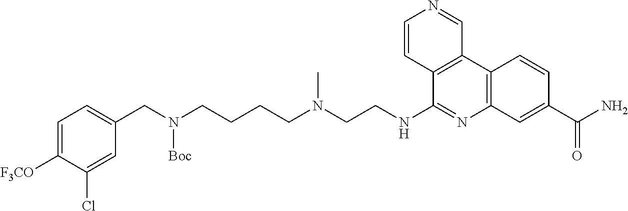 [SMILES: CN(CCCCN(Cc1ccc(OC(F)(F)F)c(Cl)c1)C(=O)OC(C)(C)C)CCNc1nc2cc(C(N)=O)ccc2c2cnccc12]

To a solution of compound 1.162 (50 mg, 169.30 μmol) in MeOH (5 mL) was added DIPEA (44 mg, 340.44 μmol) and Intermediate I (67.01 mg, 169.30 μmol) at 20° C. The resulting mixture was stirred at 20° C. for 14 h, then NaBH(OAc)₃ (72 mg, 339.72 μmol) was added. The mixture was stirred for 1 h. The mixture was filtered, concentrated in vacuo and purified (PM38) to afford compound 1.163 (50 mg, 63.36 μmol, 37.4% yield, TFA salt) as yellow solid.

LCMS (AM3): rt=0.769 min, (675.3 [M+H]⁺), 50.5% purity.

### Synthesis of Intermediate 1.164

### tert-Butyl (2-((8-carbamoylbenzo[c][2,6]naphthyridin-5-yl)amino)ethyl) (4-((3-chloro-4-(pyridin-2-yl)benzyl)amino)butyl)carbamate 1.164

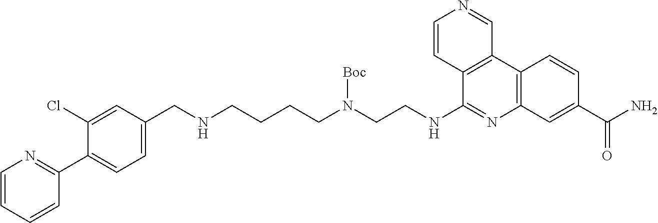 [SMILES: CC(C)(C)OC(=O)N(CCCCNCc1ccc(-c2ccccn2)c(Cl)c1)CCNc1nc2cc(C(N)=O)ccc2c2cnccc12]

To a mixture of compound 1.104 (100 mg, 0.135 mmol, TFA salt) in 1,4-dioxane (8 mL) and H₂O (2 mL) was added K₂CO₃ (80 mg, 0.579 mmol) followed by addition of 2-bromopyridine (26 μL, 0.273 mmol) and Pd(dppf)Cl₂ (10 mg, 0.014 mmol) at RT. The reaction was degassed and purged with N₂ (×3), then it was heated to 80° C. and stirred for 16 h. The reaction mixture was filtered and the filtrate was concentrated in vacuo. The crude product was purified (PM40) to afford compound 1.164 (38 mg, 36% yield, TFA salt) as a yellow solid.

LCMS (AM4): rt=0.666 min, (654.2 [M+H]⁺), 96.3% purity.

### Synthesis of Intermediate 1.166

### 4-methyl-5-phenylpicolinaldehyde 1.166

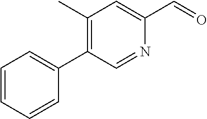

To a mixture of phenylboronic acid (100 mg, 0.82 mmol) in 1,4-dioxane (8 mL) and H₂O (2 mL) was added K₂CO₃ (172 mg, 1.24 mmol) followed by addition of 6-bromo-5-methylnicotinaldehyde (100 mg, 0.5 mmol) and Pd(dppf)Cl₂ (36 mg, 0.049 mmol) at RT. The reaction was degassed and purged with N₂ (×3), then it was heated to 80° C. and stirred for 2 h. The reaction mixture was poured into H₂O (50 mL) and the resulting mixture was extracted with EA (25 mL×2). The combined organic phases were washed (brine, 50 mL), dried (Na₂SO₄), filtered and concentrated in vacuo. The crude product was purified (PM12) to afford compound 1.166 (84 mg, 84.9% yield) as a brown oil.

LCMS (AM3): rt=0.602 min, (198.1 [M+H]⁺), 99.7% purity.

### Synthesis of Intermediate J

### N-((2-Chloro-[1,1′-biphenyl]-4-yl)methyl)but-3-yn-1-amine, 1.169

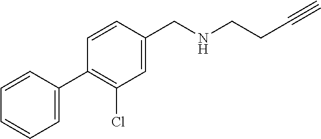 [SMILES: C#CCCNCc1ccc(-c2ccccc2)c(Cl)c1]

A mixture of but-3-yn-1-amine hydrochloride (974.47 mg, 9.23 mmol, HCl salt), NaOAc (946.56 mg, 11.54 mmol) and 3-chloro-4-phenylbenzaldehyde (CAS 57592-44-6, 500 mg, 2.31 mmol) in MeOH (20 mL) was stirred at 20° C. for 12 h, then NaBH(OAc)₃ (1.96 g, 9.23 mmol) was added. The mixture was stirred at 20° C. for 3 h. The mixture was filtered, and the filtrate was concentrated in vacuo. The residue was purified (PM46) to afford compound 1.169 (500 mg, 1.85 mmol, 80.3% yield) as a yellow oil.

LCMS (AM3): rt=0.794 min, (270.5 [M+H]⁺), 100% purity.

### tert-Butyl but-3-yn-1-yl((2-chloro-[1,1′-biphenyl]-4-yl)methyl)carbamate, Intermediate J

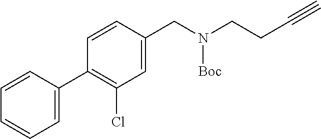 [SMILES: C#CCCN(Cc1ccc(-c2ccccc2)c(Cl)c1)C(=O)OC(C)(C)C]

To a mixture of compound 1.169 (500 mg, 1.85 mmol) and TEA (562.65 mg, 5.56 mmol) in THF (10 ml) was added Boc₂O (444.96 mg, 2.04 mmol) at 20° C. The mixture was stirred at 20° C. for 12 h. The mixture was concentrated in vacuo and purified (PM6) to afford Intermediate J (370 mg, 1.00 mmol, 54% yield) as a colourless oil.

¹H NMR (400 MHz, CHCl₃-d) δ: 7.48-7.29 (m, 7H), 7.19 (br s, 1H), 4.55 (s, 2H), 3.51-3.33 (m, 2H), 2.52-2.40 (m, 2H), 2.00 (t, J=2.4 Hz, 1H), 1.54-1.45 (m, 9H) ppm.

### Synthesis of Intermediate 1.172

### Methyl 5-((2-azidoethyl)amino)benzo[c][2,6]naphthyridine-8-carboxylate 1.170

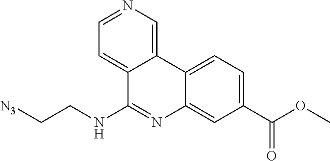 [SMILES: COC(=O)c1ccc2c(c1)nc(NCCN=[N+]=[N-])c1ccncc12]

To a mixture of 2-azidoethanamine hydrochloride (205.23 mg, 2.38 mmol) and compound 1.1 (500 mg, 1.61 mmol) in DMSO (3 mL) was added DIPEA (710.94 mg, 5.50 mmol) at RT. The mixture was heated to 80° C. and stirred for 16 h. The mixture was poured into a solution of H₂O (40 mL) and EtOH (10 mL), and the resulting precipitate was collected by filtration and dried under vacuum to afford compound 1.170 (550 mg, 1.71 mmol, 93% yield) as a grey solid.

LCMS (AM3): rt=0.782 min, (323.4 [M+H]⁺), 74.8% purity.

### methyl 5-((2-(4-(2-((tert-Butoxycarbonyl) ((2-chloro-[1,1′-biphenyl]-4-yl)methyl)amino)ethyl)-1H-1,2,3-triazol-1-yl)ethyl)amino)benzo[c][2,6]naphthyridine-8-carboxylate 1.171

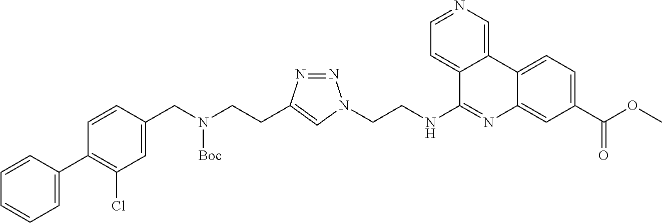 [SMILES: COC(=O)c1ccc2c(c1)nc(NCCn1cc(CCN(Cc3ccc(-c4ccccc4)c(Cl)c3)C(=O)OC(C)(C)C)nn1)c1ccncc12]

To a mixture of Intermediate J (150 mg, 0.405 mmol) and compound 1.170 (131 mg, 0.406 mmol) in DMF (3.5 mL) and MeOH (0.5 mL) was added CuI (20 mg, 0.105 mmol) at RT. The mixture was heated to 100° C. and stirred for 16 h under N₂. The mixture was diluted with H₂O (30 mL) and the aq. phase was extracted with EA (20 mL×3). The combined organic phases were washed (brine, 50 mL), dried (Na₂SO₄), filtered and concentrated in vacuo. The residue was purified (PM40) to afford compound 1.171 (240 mg, 0.347 mmol, 85.5% yield) as a brown solid.

LCMS (AM3): rt=1.059 min, (692.0 [M+H]⁺), 87.3% purity.

### tert-Butyl (2-(1-(2-((8-carbamoylbenzo[c][2,6]naphthyridin-5-yl)amino)ethyl)-1H-1,2,3-triazol-4-yl)ethyl)((2-chloro-[1,1′-biphenyl]-4-yl)methyl)carbamate 1.172

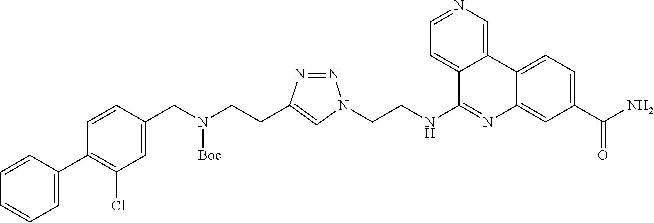 [SMILES: CC(C)(C)OC(=O)N(CCc1cn(CCNc2nc3cc(C(N)=O)ccc3c3cnccc23)nn1)Cc1ccc(-c2ccccc2)c(Cl)c1]

A solution of compound 1.171 (240 mg, 346.72 μmol) in a solution of NH₃ in MeOH (7 M, 5 mL) was stirred at 80° C. for 12 h in a sealed tube. The mixture was concentrated in vacuo to afford compound 1.172 (230 mg) as a yellow solid, which was used directly without purification.

LCMS (AM3): rt=0.943 min, (677.1 [M+H]⁺), 71.8% purity.

### Synthesis of Intermediate K

### Methyl 5-((2-(4-(2-((tert-butoxycarbonyl)amino)ethyl)-1H-1,2,3-triazol-1-yl)ethyl)amino) benzo[c][2,6]naphthyridine-8-carboxylate, 1.173

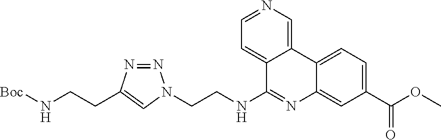 [SMILES: COC(=O)c1ccc2c(c1)nc(NCCn1cc(CCNC(=O)OC(C)(C)C)nn1)c1ccncc12]

To a mixture of compound 1.170 (2.6 g, 8.07 mmol) in DMF (70 mL) and MeOH (10 mL) was added tert-butyl but-3-yn-1-ylcarbamate (1.37 g, 8.07 mmol) and CuI (307.25 mg, 1.61 mmol) at RT. The reaction mixture was heated to 100° C. and stirred for 12 h under N₂. The mixture was diluted with H₂O (100 mL) and the aq. phase was extracted with EA (100 mL×3). The combined organic phases were washed (brine, 200 mL), dried (Na₂SO₄), filtered and concentrated in vacuo. The residue was purified (PM46) to afford compound 1.173 (3.7 g, 7.15 mmol, 88.7% yield) as a yellow solid.

LCMS (AM3): rt=0.839 min, (492.5 [M+H]⁺), 97.8% purity.

### 5-((2-(4-(2-((tert-Butoxycarbonyl)amino)ethyl)-1H-1,2,3-triazol-1-yl)ethyl)amino) benzo[c][2,6]naphthyridine-8-carboxylic acid, 1.174

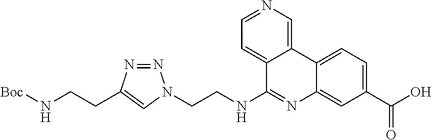 [SMILES: CC(C)(C)OC(=O)NCCc1cn(CCNc2nc3cc(C(=O)O)ccc3c3cnccc23)nn1]

To a mixture of compound 1.173 (3.7 g, 7.15 mmol) in THF (30 mL), MeOH (30 mL) and H₂O (30 mL) was added LiOH·H₂O (1.50 g, 35.75 mmol) at 20° C. The reaction mixture was stirred at 20° C. for 12 h. The solvent was removed in vacuo and the aq. was acidified with aq. HCl to pH3. The resulting precipitate was collected by filtration and dried under vacuum to afford compound 1.174 (2.2 g, 4.61 mmol, 64.4% yield) as a yellow solid.

LCMS (AM3): rt=0.785 min, (478.5 [M+H]⁺), 94.2% purity.

### tert-Butyl (2-(1-(2-((8-carbamoylbenzo[c][2,6]naphthyridin-5-yl)amino)ethyl)-1H-1,2,3-triazol-4-yl)ethyl)carbamate, 1.175

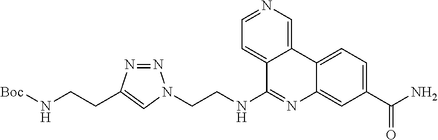 [SMILES: CC(C)(C)OC(=O)NCCc1cn(CCNc2nc3cc(C(N)=O)ccc3c3cnccc23)nn1]

To a solution of compound 1.174 (2.2 g, 4.61 mmol) in DMF (50 mL) was added HATU (2.10 g, 5.53 mmol) and DIPEA (1.79 g, 13.82 mmol, 2.41 mL) at 20° C. After being stirred for 30 min, NH₄Cl (739.33 mg, 13.82 mmol) was added. The reaction mixture was stirred at 20° C. for 1.5 h. The reaction mixture was diluted with H₂O (60 mL) and then extracted with EA (100 mL×3). The combined organic layer was washed (brine, 80 mL×3), dried (Na₂SO₄), filtered and concentrated in vacuo to afford compound 1.175 (2.5 g) as a yellow solid, which was used directly without further purification.

LCMS (AM3): rt=0.746 min, (477.5 [M+H]⁺), 77.1% purity.

### 5-((2-(4-(2-Aminoethyl)-1H-1,2,3-triazol-1-yl)ethyl)amino)benzo[c][2,6]naphthyridine-8-carboxamide, Intermediate K

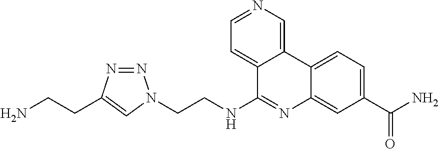 [SMILES: NCCc1cn(CCNc2nc3cc(C(N)=O)ccc3c3cnccc23)nn1]

To a solution of compound 1.175 (2.5 g, 5.25 mmol) in MeOH (10 mL) was added a solution of HCl in MeOH (4 M, 20 mL) slowly at 0° C. The reaction mixture was stirred at 20° C. for 12 h. The reaction mixture was concentrated in vacuo to afford Intermediate K (1.2 g) as a yellow solid, which was used directly without purification.

LCMS (AM5): rt=0.698 min, (377.5 [M+H]⁺), 94.4% purity.

### Synthesis of Intermediate 1.184

### Ethyl 4-(3-((tert-butoxycarbonyl)((2-chloro-[1,1′-biphenyl]-4-yl)methyl)amino)propanamido)-3-oxobutanoate 1.177

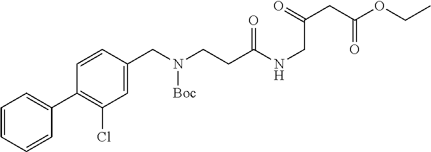 [SMILES: CCOC(=O)CC(=O)CNC(=O)CCN(Cc1ccc(-c2ccccc2)c(Cl)c1)C(=O)OC(C)(C)C]

To a mixture of Intermediate D (2.07 g, 5.31 mmol) and DIPEA (2.06 g, 15.91 mmol) in DMF (20 mL) was added HATU (2.42 g, 6.36 mmol) at 20° C. After stirring at 20° C. for 0.5 h, ethyl 4-amino-3-oxobutanoate (963 mg, 5.30 mmol, HCl salt) (ref: EP1650201) was added. The mixture was stirred at 20° C. for 15 h. The mixture was diluted with H₂O (20 mL) and the aq. phase was extracted with EA (20 mL×3). The combined organic phase was washed (brine, 50 mL×3), dried (Na₂SO₄), filtered and concentrated in vacuo. The residue was purified (PM55) to afford compound 1.177 (1.5 g, 2.90 mmol, 54.7% yield) as a yellow oil.

LCMS (AM3): rt=1.010 min, (539.3 [M+Na]⁺), 100% purity.

### Ethyl 2-(2-(2-((tert-butoxycarbonyl)((2-chloro-[1,1′-biphenyl]-4-yl)methyl)amino)ethyl)oxazol-5-yl)acetate 1.178

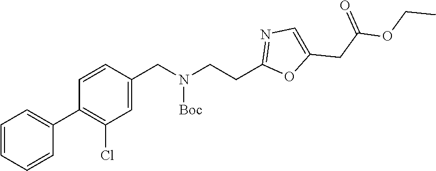 [SMILES: CCOC(=O)Cc1cnc(CCN(Cc2ccc(-c3ccccc3)c(Cl)c2)C(=O)OC(C)(C)C)o1]

To a mixture of compound 1.177 (750 mg, 1.45 mmol) in DMF (2 mL) was added POCl₃ (480 mg, 3.13 mmol) at RT. The reaction was heated to 60° C. and stirred for 0.5 h under N₂. The reaction mixture was quenched with saturated NaHCO₃ (20 mL) and the aq. phase was extracted with EA (20 mL×2). The combined organic phase was washed (brine, 30 mL×2), dried (Na₂SO₄), filtered and concentrated in vacuo. The residue was purified (PM6) to afford compound 1.178 (520 mg, 1.04 mmol, 35.9% yield) as a yellow oil.

¹H NMR (400 MHz, CHCl₃-d) δ: 7.45-7.37 (m, 5H), 7.36-7.28 (m, 2H), 7.17 (br s, 1H), 6.87 (s, 1H), 4.45-4.35 (br m, 2H), 4.24-4.18 (q, 2H), 3.70-3.60 (m, 4H), 3.10-2.95 (br m, 2H), 1.55-1.43 (br s, 9H), 1.28 (t, J=7.2 Hz, 3H) ppm.

### 2-(2-(2-((tert-butoxycarbonyl)((2-chloro-[1,1′-biphenyl]-4-yl)methyl)amino)ethyl)oxazol-5-yl) acetic acid 1.179

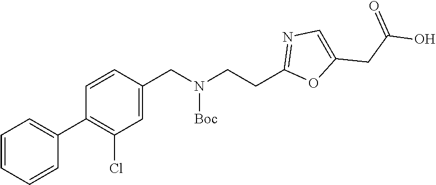 [SMILES: CC(C)(C)OC(=O)N(CCc1ncc(CC(=O)O)o1)Cc1ccc(-c2ccccc2)c(Cl)c1]

To a mixture of compound 1.178 (520 mg, 1.04 mmol) in THF (9 mL), MeOH (1 mL) and H₂O (1 mL) was added LiOH·H₂O (437.26 mg, 10.42 mmol) at 20° C. The reaction was stirred at 20° C. for 16 h. The mixture was adjusted to pH 6 by adding aq. HCl (1 N) and the aq. phase was extracted with EA (15 mL×3). The combined organic phase was washed (brine, 30 mL), dried (Na₂SO₄), filtered and concentrated in vacuo to afford compound 1.179 (510 mg) as a yellow oil, which was used directly without purification.

LCMS (AM3): rt=1.003 min, (471.3 [M+H]⁺), 93.9% purity.

### tert-butyl(2-(5-(2-amino-2-oxoethyl)oxazol-2-yl)ethyl)((2-chloro-[1,1′-biphenyl]-4-yl)methyl)carbamate 1.180

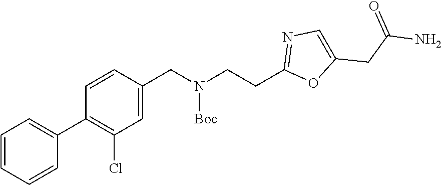 [SMILES: CC(C)(C)OC(=O)N(CCc1ncc(CC(N)=O)o1)Cc1ccc(-c2ccccc2)c(Cl)c1]

To a mixture of compound 1.179 (510 mg, 1.08 mmol) in THF (10 mL) was added HATU (494.12 mg, 1.30 mmol) and DIPEA (419.88 mg, 3.25 mmol) at 20° C. After stirring at 20° C. for 0.5 h, NH₄Cl (173.78 mg, 3.25 mmol) was added. The mixture was warmed to 35° C. and stirred for 2 h. The reaction mixture was diluted with H₂O (30 mL) and the aq. phase was extracted with EA (30 mL×2). The combined organic phase was washed (brine, 50 mL), dried (Na₂SO₄), filtered and concentrated in vacuo to afford compound 1.180 (450 mg) as a yellow oil, which was used directly without purification.

### tert-butyl((2-chloro-[1,1′-biphenyl]-4-yl)methyl)(2-(5-(cyanomethyl)oxazol-2-yl)ethyl)carbamate 1.181

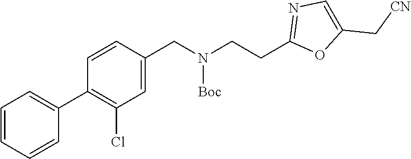 [SMILES: CC(C)(C)OC(=O)N(CCc1ncc(CC#N)o1)Cc1ccc(-c2ccccc2)c(Cl)c1]

To a mixture of compound 1.180 (400 mg, 851.14 μmol) in THF (10 mL) was added Burgess reagent (608.50 mg, 2.55 mmol) at 20° C. The reaction was stirred at 20° C. for 0.5 h. The reaction mixture was concentrated in vacuo and the obtained residue was purified (PM2) to afford compound 1.181 (330 mg, 730.18 μmol, 85.8% yield) as a yellow oil.

LCMS (AM3): rt=1.061 min, (452.3 [M+H]⁺), purity: 93.35%.

### tert-butyl(2-(5-(2-aminoethyl)oxazol-2-yl)ethyl)((2-chloro-[1,1′-biphenyl]-4-yl)methyl)carbamate 1.182

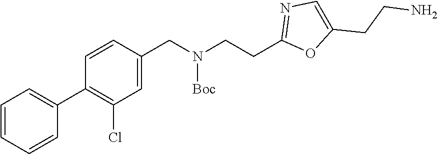 [SMILES: CC(C)(C)OC(=O)N(CCc1ncc(CCN)o1)Cc1ccc(-c2ccccc2)c(Cl)c1]

To a solution of compound 1.181 (330 mg, 730.18 μmol) and NH₃·H₂O (910 mg, 7.79 mmol, 1 mL, 30%) in MeOH (10 mL) was added Raney Ni (30 mg) under N₂. The suspension was degassed under vacuum and purged with H₂ three times. The reaction mixture was hydrogenated under 15 psi at 20° C. for 12 h. The catalyst was removed by filtration and the filtrate was concentrated in vacuo to afford compound 1.182 (310 mg) as a yellow oil, which was used directly without purification.

LCMS (AM3): rt=0.867 min, (456.3 [M+H]⁺), purity: 87.6%.

### Methyl 5-((2-(2-(2-((tert-butoxycarbonyl)((2-chloro-[1,1′-biphenyl]-4-yl)methyl)amino)ethyl)oxazol-5-yl)ethyl)amino)benzo[c][2,6]naphthyridine-8-carboxylate 1.183

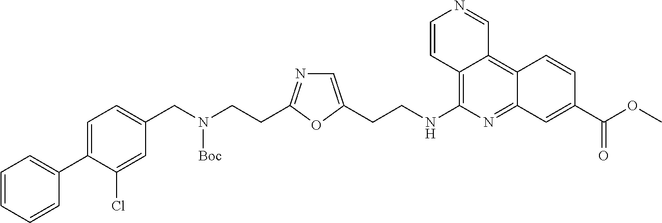 [SMILES: COC(=O)c1ccc2c(c1)nc(NCCc1cnc(CCN(Cc3ccc(-c4ccccc4)c(Cl)c3)C(=O)OC(C)(C)C)o1)c1ccncc12]

To a mixture of compound 1.182 (260 mg, 570.21 μmol) and compound 1.1 (176.69 mg, 570.21 μmol) in DMSO (5 mL) was added DIPEA (147.39 mg, 1.14 mmol) at RT. The mixture was heated to 70° C. and stirred for 16 h. The reaction mixture was diluted with H₂O (30 mL) and the aq. phase was extracted with EA (30 mL×3). The combined organic phase was washed (brine, 80 mL), dried (Na₂SO₄), filtered and concentrated in vacuo. The residue was purified (PM40) to afford compound 1.183 (200 mg, 288.93 μmol, 50.7% yield) as a yellow solid.

LCMS (AM3): rt=1.016 min, (691.9 [M+H]⁺), purity: 95.5%.

### tert-butyl (2-(5-(2-((8-carbamoylbenzo[c][2,6]naphthyridin-5-yl)amino)ethyl)oxazol-2-yl)ethyl)((2-chloro-[1,1′-biphenyl]-4-yl)methyl)carbamate 1.184

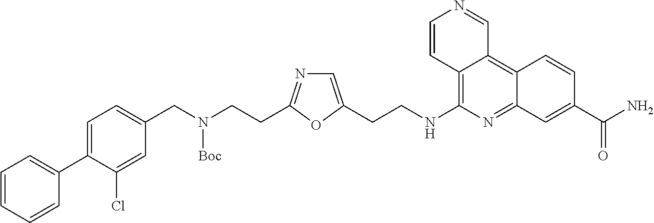 [SMILES: CC(C)(C)OC(=O)N(CCc1ncc(CCNc2nc3cc(C(N)=O)ccc3c3cnccc23)o1)Cc1ccc(-c2ccccc2)c(Cl)c1]

A solution of compound 1.183 (100 mg, 144.47 μmol) in NH₃ in MeOH solution (7 M, 5 mL) was stirred at 80° C. for 16 h in a sealed tube. The reaction mixture was concentrated in vacuo to afford compound 1.184 (100 mg) as a yellow solid, which was used directly without purification.

LCMS (AM3): rt=0.916 min, (677.0 [M+H]⁺), purity: 76.6%.

### Synthesis of Intermediate 1.187

### Methyl 5-((2-(2-(2-aminoethoxy)ethoxy)ethyl)amino)benzo[c][2,6]naphthyridine-8-carboxylate 1.186

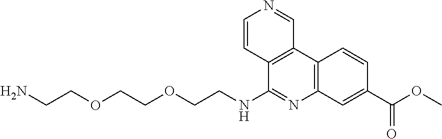 [SMILES: COC(=O)c1ccc2c(c1)nc(NCCOCCOCCN)c1ccncc12]

To a mixture of compound 1.1 (500 mg, 1.83 mmol) and 2,2′-(ethane-1,2-diylbis(oxy))diethanamine (543.49 mg, 3.67 mmol) in DMSO (10 mL) was added DIPEA (710.94 mg, 5.50 mmol) at RT. The mixture was heated to 80° C. and stirred for 12 h. The mixture was concentrated in vacuo and the obtained residue was purified (PM40) to afford compound 1.186 (150 mg, 304.35 μmol, 16.6% yield) as a light yellow solid.

LCMS (AM3): rt=0.675 min, (385.2 [M+H]⁺), 65.4% purity.

### 5-((2-(2-(2-Aminoethoxy)ethoxy)ethyl)amino)benzo[c][2,6]naphthyridine-8-carboxamide 1.187

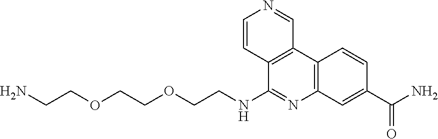 [SMILES: NCCOCCOCCNc1nc2cc(C(N)=O)ccc2c2cnccc12]

A solution of compound 1.186 (150 mg, 304.35 μmol) in saturated NH₃ in MeOH solution (7 M, 10 mL) was stirred at 85° C. for 12 h in a sealed tube. The reaction mixture was concentrated in vacuo to afford compound 1.187 (140 mg) as yellow oil, which was used directly without further purification.

LCMS (AM3): rt=0.532 min, (370.1 [M+H]⁺), 48.7% purity.

### Synthesis of Intermediate 1.192

### 3-(((2-Chloro-[1,1′-biphenyl]-4-yl)methyl)amino)propanenitrile 1.188

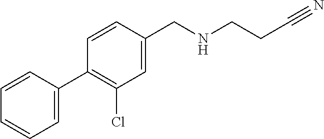 [SMILES: N#CCCNCc1ccc(-c2ccccc2)c(Cl)c1]

A mixture of 3-chloro-4-phenylbenzaldehyde (CAS 57592-44-6, 600 mg, 2.77 mmol) and 3-aminopropanenitrile (240 mg, 3.42 mmol) in MeOH (5 mL) was stirred at 20° C. for 6 h, then NaBH(OAc)₃ (2.35 g, 11.08 mmol) was added. The reaction mixture was stirred at 20° C. for 12 h. The mixture was concentrated in vacuo and the residue was purified (PM46) to afford compound 1.188 (1.2 g) as a white solid, which was used directly without purification.

LCMS (AM3): rt=0.766 min, (271.5 [M+H]⁺), 78.56% purity.

### tert-Butyl ((2-chloro-[1,1′-biphenyl]-4-yl)methyl)(2-cyanoethyl)carbamate 1.189

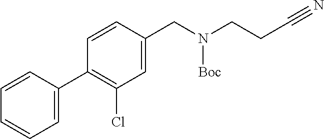 [SMILES: CC(C)(C)OC(=O)N(CCC#N)Cc1ccc(-c2ccccc2)c(Cl)c1]

To a mixture of compound 1.188 (1.2 g, 4.43 mmol) and TEA (896.95 mg, 8.86 mmol) in THF (20 mL) was added Boc₂O (967.28 mg, 4.43 mmol) at 20° C. The reaction mixture was stirred at 20° C. for 12 h. The mixture was concentrated in vacuo and the residue was purified (PM6) to afford compound 1.189 (750 mg, 2.02 mmol, 45.6% yield) as a colourless oil.

¹H NMR (400 MHz, MeOH-d₄) δ: 7.45-7.25 (m, 8H), 4.56 (s, 2H), 3.65-3.52 (br m, 2H), 2.78-2.65 (br m, 2H), 1.54-1.45 (m, 9H) ppm.

### tert-Butyl ((2-chloro-[1,1′-biphenyl]-4-yl)methyl)(3-(hydroxyamino)-3-iminopropyl)carbamate 1.190

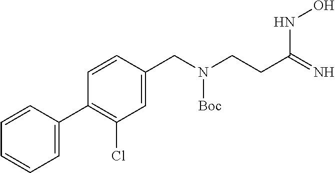 [SMILES: CC(C)(C)OC(=O)N(CCC(=N)NO)Cc1ccc(-c2ccccc2)c(Cl)c1]

To a mixture of compound 1.189 (400 mg, 1.08 mmol) and hydroxylamine hydrochloride (299.8 mg, 4.31 mmol) in EtOH (20 mL) was added Na₂CO₃ (571.5 mg, 5.39 mmol) at RT. The mixture was then heated to 80° C. and stirred for 16 h. The mixture was concentrated in vacuo and the residue was diluted with H₂O (30 mL). The aq. phase was extracted with EA (30 mL×2). The combined organic phase was washed (brine, 50 mL), dried (Na₂SO₄), filtered and concentrated in vacuo to afford compound 1.190 (400 mg, 990.34 μmol, 91.8% yield) as a colourless oil, which was used directly without further purification.

LCMS (AM3): rt=0.859 min, (404.4 [M+H]⁺), 76.6% purity.

### Methyl 5-((2-(3-(2-((tert-butoxycarbonyl)((2-chloro-[1,1′-biphenyl]-4-yl)methyl)amino)ethyl)-1,2,4-oxadiazol-5-yl)ethyl)amino)benzo[c][2,6]naphthyridine-8-carboxylate 1.191

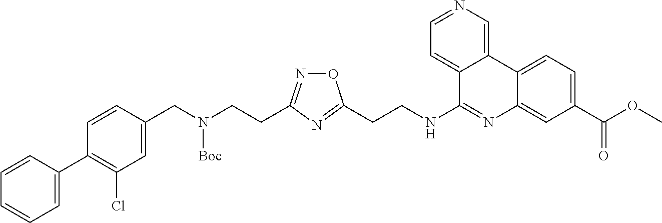 [SMILES: COC(=O)c1ccc2c(c1)nc(NCCc1nc(CCN(Cc3ccc(-c4ccccc4)c(Cl)c3)C(=O)OC(C)(C)C)no1)c1ccncc12]

A mixture of Intermediate L (100 mg, 307.39 μmol), HATU (175.32 mg, 461.09 μmol) and DIPEA (119.18 mg, 922.17 μmol) in DMF (1 mL) was stirred at 35° C. for 0.5 h, then compound 1.190 (124.16 mg, 307.39 μmol) was added. The resulting mixture was stirred at 35° C. for 2 h. The reaction mixture was diluted with H₂O (20 mL) and the aq. phase was extracted with EA (30 mL×2). The combined organic phase was washed (brine, 50 mL), dried (Na₂SO₄), filtered and concentrated in vacuo to give the intermediate product. The intermediate product was then re-dissolved in DMF (8 mL) and heated to 110° C. for 16 h. The reaction mixture was concentrated in vacuo and the residue was purified (PM57) to afford compound 1.191 (160 mg, 230.82 μmol, 82% yield) as a yellow solid.

LCMS (AM3): rt=1.056 min, (692.9 [M+H]⁺), purity: 91.9%.

### tert-Butyl (2-(5-(2-((8-carbamoylbenzo[c][2,6]naphthyridin-5-yl)amino)ethyl)-1,2,4-oxadiazol-3-yl)ethyl)((2-chloro-[1,1′-biphenyl]-4-yl)methyl)carbamate 1.192

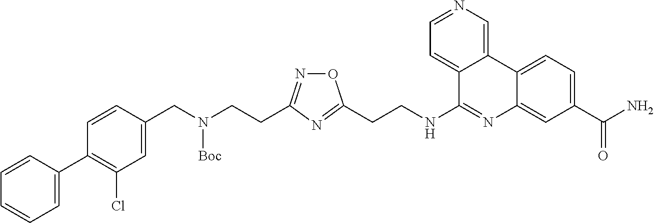 [SMILES: CC(C)(C)OC(=O)N(CCc1noc(CCNc2nc3cc(C(N)=O)ccc3c3cnccc23)n1)Cc1ccc(-c2ccccc2)c(Cl)c1]

A mixture of compound 1.191 (150 mg, 216.39 μmol) in saturated NH₃ in MeOH solution (10 mL) was stirred at 80° C. for 16 h in a sealed tube. The reaction mixture was concentrated in vacuo to afford compound 1.192 (140 mg) as a yellow solid, which was used directly without further purification.

LCMS (AM3): rt=0.938 min, (678.0 [M+H]⁺), purity: 58.8%.

### Synthesis of Intermediate 1.195

### tert-Butyl ((2-chloro-[1,1′-biphenyl]-4-yl)methyl)(3-hydrazinyl-3-iminopropyl)carbamate 1.193

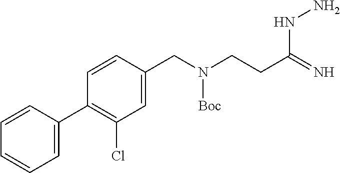 [SMILES: CC(C)(C)OC(=O)N(CCC(=N)NN)Cc1ccc(-c2ccccc2)c(Cl)c1]

A mixture of compound 1.189 (600 mg, 1.62 mmol) and hydrazine hydrate (809.88 mg, 16.18 mmol) in a freshly prepared methanolic solution of NaOMe (2.45 M, 10 mL) was stirred at 70° C. for 5 h. The reaction mixture was acidified with FA and concentrated in vacuo. The residue was purified (PM58) to afford compound 1.193 (180 mg, 446.74 μmol, 27.6% yield) as a yellow solid.

LCMS (AM3): rt=0.861 min, (403.1 [M+H]⁺), 99.7% purity.

### Methyl 5-((2-(5-(2-((tert-butoxycarbonyl)((2-chloro-[1,1′-biphenyl]-4-yl)methyl)amino)ethyl)-4H-1,2,4-triazol-3-yl)ethyl)amino)benzo[c][2,6]naphthyridine-8-carboxylate 1.194

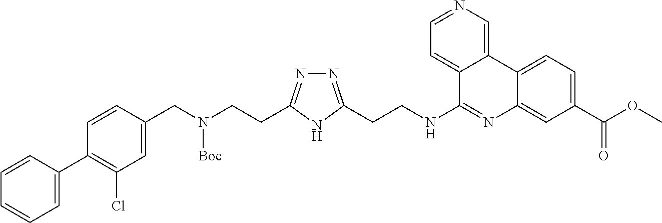 [SMILES: COC(=O)c1ccc2c(c1)nc(NCCc1nnc(CCN(Cc3ccc(-c4ccccc4)c(Cl)c3)C(=O)OC(C)(C)C)[nH]1)c1ccncc12]

A mixture of Intermediate L (121.11 mg, 372.29 μmol), HATU (212.33 mg, 558.43 μmol) and DIPEA (144.34 mg, 1.12 mmol) in DMF (5 mL) was stirred at 35° C. for 0.5 h, then compound 1.193 (150 mg, 372.29 μmol) was added. The reaction mixture was stirred at 35° C. for 2 h, then heated to 110° C. and stirred for 12 h. The reaction mixture was diluted with H₂O (20 mL) and the aq. phase was extracted with EA (30 mL×2). The combined organic phase was washed (brine, 50 mL), dried (Na₂SO₄), filtered and concentrated in vacuo. The obtained residue was purified (PM59) to afford compound 1.194 (110 mg, 158.91 μmol, 42.7% yield) as a yellow solid.

LCMS (AM3): rt=0.944 min, (692.2 [M+H]⁺), 100% purity.

### tert-Butyl (2-(5-(2-((8-carbamoylbenzo[c][2,6]naphthyridin-5-yl)amino)ethyl)-4H-1,2,4-triazol-3-yl)ethyl)((2-chloro-[1,1′-biphenyl]-4-yl)methyl)carbamate 1.195

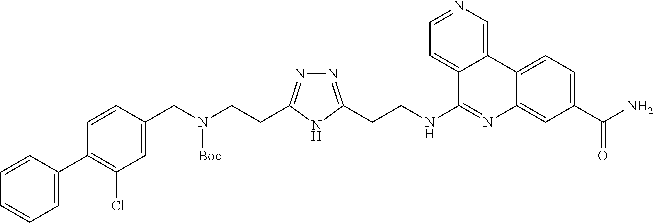 [SMILES: CC(C)(C)OC(=O)N(CCc1nnc(CCNc2nc3cc(C(N)=O)ccc3c3cnccc23)[nH]1)Cc1ccc(-c2ccccc2)c(Cl)c1]

A mixture of compound 1.194 (110 mg, 158.91 μmol) in Ammonia in MeOH (7 M, 8 mL) was stirred at 80° C. for 16 h in a sealed tube. The reaction mixture was concentrated in vacuo to afford compound 1.195 (100 mg) as a white solid, which was used directly without purification.

LCMS (AM3): rt=0.878 min, (677.0 [M+H]⁺), 73.3% purity.

### Synthesis of Intermediate 1.201

### 4-((4-Bromo-3-chlorobenzyl)amino) butan-1-ol 1.197

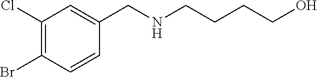 [SMILES: OCCCCNCc1ccc(Br)c(Cl)c1]

A mixture of 4-bromo-3-chloro-benzaldehyde (10 g, 45.57 mmol) and 4-aminobutan-1-ol (4.06 g, 45.55 mmol) in MeOH (200 mL) was stirred at RT for 2 h, then NaBH(OAc)₃ (38.63 g, 182.26 mmol) added. The reaction mixture was stirred at RT for 2 h. The reaction mixture was filtered and the filtrate was concentrated in vacuo. The residue was dissolved in EA (200 mL) and then filtered. The filtrate was concentrated in vacuo to afford compound 1.197 (20.4 g) as a light brown oil, which was used directly without further purification.

LCMS (AM3): rt=0.525 min, (294.0 [M+H]⁺), 47.0% purity.

### tert-Butyl 4-bromo-3-chlorobenzyl(4-hydroxybutyl)carbamate 1.198

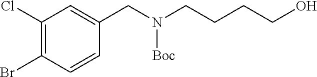 [SMILES: CC(C)(C)OC(=O)N(CCCCO)Cc1ccc(Br)c(Cl)c1]

To mixture of compound 1.197 (20.4 g, 69.72 mmol) in THF (100 mL) and H₂O (100 mL) was added NaHCO₃ (11.71 g, 139.44 mmol) followed by Boc₂O (20 mL, 87.06 mmol) at RT. The reaction mixture was stirred at RT for 2 h. The reaction mixture was filtered, the filtrate was diluted with H₂O (100 mL) and extracted with EA (50 mL×2). The combined organic phase was washed (brine, 100 mL), dried (Na₂SO₄), filtered and concentrated in vacuo. The residue was purified (PM3) to afford compound 1.198 (14.1 g, 44.8% yield) as a colorless oil.

LCMS (AM4): rt=1.074 min, (414.0, [M+Na]⁺), 87.3% purity.

### tert-Butyl 4-bromo-3-chlorobenzyl(4-oxobutyl)carbamate 1.199

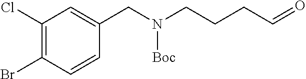 [SMILES: CC(C)(C)OC(=O)N(CCCC=O)Cc1ccc(Br)c(Cl)c1]

To a solution of compound 1.198 (14.1 g, 31.24 mmol) in DCM (150 mL) was added DMP (16 g, 37.72 mmol) in portions at RT. The reaction mixture was stirred at RT for 17 h. The reaction mixture was filtered and the filtrate was concentrated in vacuo. The crude product was purified (PM6) to afford compound 1.199 (10 g, 72.9% yield) as a colorless oil.

LCMS (AM3): rt=1.049 min, (413.9, [M+Na]⁺), 59.0% purity.

### tert-Butyl 4-bromo-3-chlorobenzyl(4-((2-((8-carbamoylbenzo[c][2,6]naphthyridin-5-yl)amino)ethyl)amino)butyl)carbamate 1.200

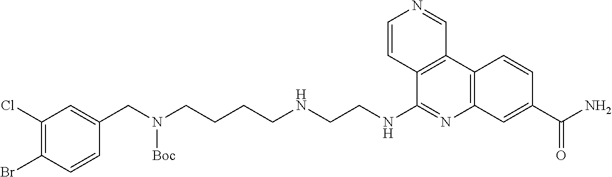 [SMILES: CC(C)(C)OC(=O)N(CCCCNCCNc1nc2cc(C(N)=O)ccc2c2cnccc12)Cc1ccc(Br)c(Cl)c1]

A mixture of compound 1.199 (3 g, 6.83 mmol), Intermediate F (4 g, 10.12 mmol, TFA salt) and DIPEA (7.50 mL, 43.06 mmol) in MeOH (200 mL) was stirred at RT for 16 h, then NaBH(OAc)₃ (5.79 g, 27.32 mmol) was added. The reaction mixture was stirred at RT for 1 h. The reaction mixture was filtered and the filtrate was concentrated in vacuo. The crude product was purified (PM60) to afford compound 1.200 (0.6 g, 10.8% yield, TFA salt) as a yellow solid.

LCMS (AM4): rt=0.765 min, (657.0 [M+H]⁺), 95.0% purity.

### tert-Butyl (4-((2-((8-carbamoylbenzo[c][2,6]naphthyridin-5-yl)amino)ethyl)amino)butyl)((2-chloro-2′-(hydroxymethyl)-[1,1′-biphenyl]-4-yl)methyl)carbamate 1.201

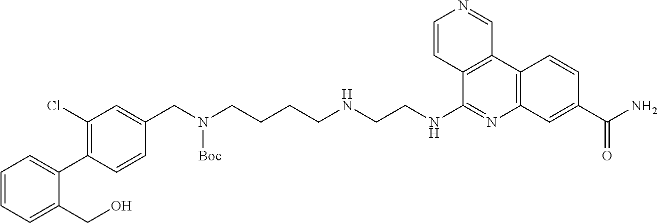 [SMILES: CC(C)(C)OC(=O)N(CCCCNCCNc1nc2cc(C(N)=O)ccc2c2cnccc12)Cc1ccc(-c2ccccc2CO)c(Cl)c1]

To a mixture of compound 1.200 (150 mg, 0.185 mmol, TFA salt) in 1,4-dioxane (8 mL) and H₂O (2 mL) was added K₂CO₃ (102 mg, 0.738 mmol) followed by addition of (2-(hydroxymethyl)phenyl) boronic acid (30 mg, 0.197 mmol) and Pd(dppf)Cl₂ (14 mg, 0.019 mmol) at RT. The reaction mixture was degassed and purged with N₂ (×3). The reaction mixture was heated to 80° C. and stirred for 3 h. The reaction mixture was filtered and the filtrate was concentrated in vacuo. The crude product was purified (PM40) to afford compound 1.201 (88 mg, 55.5% yield, TFA salt) as a yellow solid.

LCMS (AM4): rt=0.749 min, (683.2 [M+H]⁺), 80.3% purity.

### Synthesis of Intermediate 1.203

### tert-Butyl (4-((2-((8-carbamoylbenzo[c][2,6]naphthyridin-5-yl)amino)ethyl)amino)butyl)(3-chloro-4-(pyridin-3-yl)benzyl)carbamate 1.203

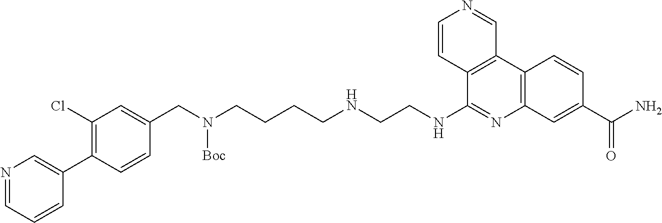 [SMILES: CC(C)(C)OC(=O)N(CCCCNCCNc1nc2cc(C(N)=O)ccc2c2cnccc12)Cc1ccc(-c2cccnc2)c(Cl)c1]

To a mixture of compound 1.200 (100 mg, 0.123 mmol, TFA salt) in 1,4-dioxane (8 mL) and H₂O (2 mL) was added K₂CO₃ (68 mg, 0.492 mmol) followed by addition of pyridin-3-ylboronic acid (16 mg, 0.130 mmol) and Pd(dppf)Cl₂ (9 mg, 0.012 mmol) at RT. The reaction mixture was degassed and purged with N₂ (×3), then it was heated to 80° C. and stirred for 15 h. The reaction mixture was filtered and the filtrate was concentrated in vacuo. The crude product was purified (PM40) to afford compound 1.203 (80 mg, 84.4% yield, TFA salt) as a yellow solid.

LCMS (AM4): rt=0.669 min, (654.2 [M+H]⁺), 99.6% purity.

### Synthesis of Intermediate 1.204

### tert-Butyl (4-((2-((8-carbamoylbenzo[c][2,6]naphthyridin-5-yl)amino)ethyl)amino)butyl)(3-chloro-4-(pyridin-4-yl)benzyl)carbamate 1.204

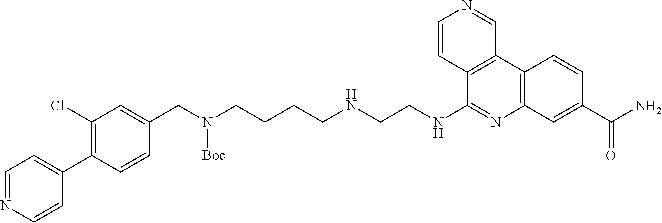 [SMILES: CC(C)(C)OC(=O)N(CCCCNCCNc1nc2cc(C(N)=O)ccc2c2cnccc12)Cc1ccc(-c2ccncc2)c(Cl)c1]

To a mixture of compound 1.200 (250 mg, 0.308 mmol, TFA salt) in 1,4-dioxane (8 mL) and H₂O (2 mL) was added K₂CO₃ (170 mg, 1.23 mmol) followed by pyridin-4-ylboronic acid (95 mg, 0.773 mmol) and Pd(dppf)Cl₂ (23 mg, 0.031 mmol) at RT. The reaction mixture was degassed and purged with N₂ (×3), then it was heated to 80° C. and stirred for 15 h. The reaction mixture was filtered and the filtrate was concentrated in vacuo. The crude product was purified (PM40) to afford compound 1.204 (84 mg, 35.5% yield, TFA salt) as a yellow solid.

LCMS (AM3): rt=0.770 min, (654.1 [M+H]⁺), 100% purity.

### Synthesis of Intermediate L

### 3-((8-(Methoxycarbonyl)benzo[c][2,6]naphthyridin-5-yl)amino)propanoic acid, Intermediate L

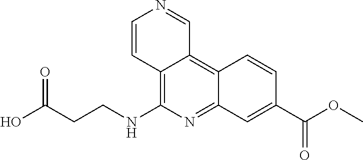 [SMILES: COC(=O)c1ccc2c(c1)nc(NCCC(=O)O)c1ccncc12]

To a mixture of 3-aminopropanoic acid (1.04 g, 11.62 mmol) and compound 1.1 (600 mg, 1.94 mmol) in DMSO (4 mL) was added DIPEA (2.23 g, 17.22 mmol) at RT. The reaction mixture was heated to 100° C. and stirred for 12 h. The mixture was concentrated in vacuo and the obtained solid was triturated with a mixture of EtOH (30 mL) and H₂O (40 mL). The resulting mixture was filtered and the solid was dried in vacuo to afford Intermediate L (400 mg, 1.23 mmol, 63.5% yield) as a grey solid.

LCMS (AM3): rt=0.711 min, (326.4 [M+H]⁺), purity: 71.9%.

### Synthesis of Intermediate M

### 4-(((2-Chloro-[1,1′-biphenyl]-4-yl)methyl)amino)butanoic acid, 1.206

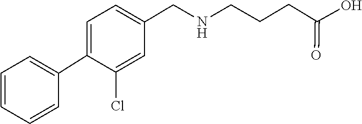 [SMILES: O=C(O)CCCNCc1ccc(-c2ccccc2)c(Cl)c1]

To a solution of 4-aminobutanoic acid (5.71 g, 55.39 mmol) in MeOH (30 mL) was added 3-chloro-4-phenylbenzaldehyde (CAS 57592-44-6, 3 g, 13.85 mmol) at 25° C. After stirring for 2 h, NaBH(OAc)₃ (8.80 g, 41.54 mmol) was added. The reaction mixture was stirred at 25° C. for 12 h. The mixture was filtered and the filtrate was concentrated and the residue purified (PM39) to afford compound 1.206 (1.9 g, 6.25 mmol, 45.2% yield) as a white solid.

LCMS (AM3): rt=0.787 min, (304.5 [M+H]⁺), 98.9% purity.

### 4-((tert-Butoxycarbonyl)((2-chloro-[1,1′-biphenyl]-4-yl)methyl)amino)butanoic acid; Intermediate M

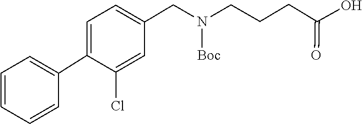 [SMILES: CC(C)(C)OC(=O)N(CCCC(=O)O)Cc1ccc(-c2ccccc2)c(Cl)c1]

To a mixture of compound 1.206 (1.5 g, 4.94 mmol) in THF (20 mL) and H₂O (2 mL) was added NaHCO₃ (829.6 mg, 9.88 mmol) and Boc₂O (1.19 g, 5.43 mmol) at 25° C. The reaction mixture was stirred at 25° C. for 2 h. The mixture was concentrated and the residue was purified (PM61) to afford Intermediate M (1.8 g, 4.46 mmol, 90.3% yield) as a white solid.

LCMS (AM3): rt=0.962 min, (426.1 [M+Na]⁺), 85.4% purity.

### Synthesis of Intermediate 1.211

### Methyl 5-((2-((tert-butoxycarbonyl)(methyl)amino)ethyl)(methyl)amino)benzo[c][2,6]naphthyridine-8-carboxylate 1.207

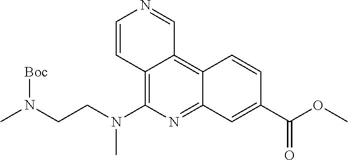

To a solution of compound 1.1 (300 mg, 1.10 mmol) in DMSO (10 mL) was added DIPEA (284.38 mg, 2.20 mmol) and tert-butyl N-methyl-N-[2-(methylamino)ethyl]carbamate (248.55 mg, 1.32 mmol) at RT. The reaction mixture was then heated to 80° C. and stirred for 12 h. The mixture was poured into H₂O (50 mL) and extracted with EA (50 mL×3). The combined organic phases were washed (brine, 50 mL), dried (Na₂SO₄) and concentrated in vacuo to afford compound 1.207 (450 mg, 1.06 mmol, 96.4% yield), which was used directly without further purification.

LCMS (AM3): rt=0.842 min, (425.2 [M+H]⁺), 81.9% purity.

### 5-((2-((tert-Butoxycarbonyl)(methyl)amino)ethyl)(methyl)amino)benzo[c][2,6]naphthyridine-8-carboxylic acid 1.208

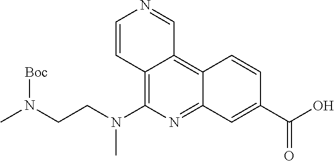 [SMILES: CN(CCN(C)c1nc2cc(C(=O)O)ccc2c2cnccc12)C(=O)OC(C)(C)C]

To a solution of compound 1.207 (400 mg, 942.30 μmol) in THF (20 mL), MeOH (2 mL) and H₂O (2 mL) was added LiOH·H₂O (395.42 mg, 9.42 mmol) at 20° C. The reaction mixture was stirred at 20° C. for 12 h. The reaction mixture was concentrated and the residue was purified (PM46) to afford compound 1.208 (300 mg, 730.88 μmol, 70.5% yield) as colourless oil.

LCMS (AM3): rt=0.754 min, (411.2 [M+H]⁺), 100% purity.

### tert-Butyl (2-((8-carbamoylbenzo[c][2,6]naphthyridin-5-yl)(methyl)amino)ethyl)(methyl)carbamate 1.209

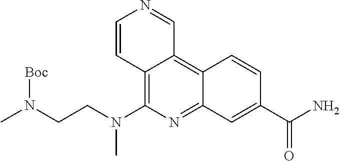 [SMILES: CN(CCN(C)c1nc2cc(C(N)=O)ccc2c2cnccc12)C(=O)OC(C)(C)C]

To a solution of compound 1.208 (280 mg, 682.15 μmol) in THF (10 mL) was added HATU (311.25 mg, 818.58 μmol) followed by DIPEA (176.32 mg, 1.36 mmol) and NH₄Cl (40.14 mg, 750.37 μmol), sequentially at 25° C. The reaction mixture was stirred at 25° C. for 12 h. The mixture was concentrated and the residue was purified (PM39) to afford compound 1.209 (160 mg, 390.74 μmol, 57.3% yield) as a yellow solid.

LCMS (AM3): rt=0.790 min, (410.1 [M+H]⁺), 96.4% purity.

### 5-(Methyl (2-(methylamino)ethyl)amino)benzo[c][2,6]naphthyridine-8-carboxamide 1.210

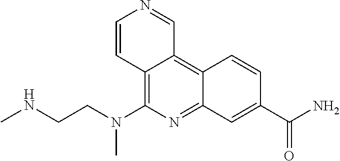 [SMILES: CNCCN(C)c1nc2cc(C(N)=O)ccc2c2cnccc12]

To a solution of compound 1.209 (140 mg, 341.90 μmol) in 1,4-dioxane (5 mL) was added a solution of HCl in 1,4-dioxane (4 M, 10 mL) at 25° C. The reaction mixture was stirred at 25° C. for 0.5 h. The mixture was concentrated in vacuo to afford compound 1.210 (100 mg, 289.16 μmol, 84.6% yield, HCl salt) as a yellow solid, which was used directly without purification.

LCMS (AM3): rt=0.374 min, (310.2 [M+H]⁺), 88.0% purity.

### tert-Butyl (4-((2-((8-carbamoylbenzo[c][2,6]naphthyridin-5-yl)(methyl)amino)ethyl)(methyl)amino)-4-oxobutyl)((2-chloro-[1,1′-biphenyl]-4-yl)methyl)carbamate 1.211

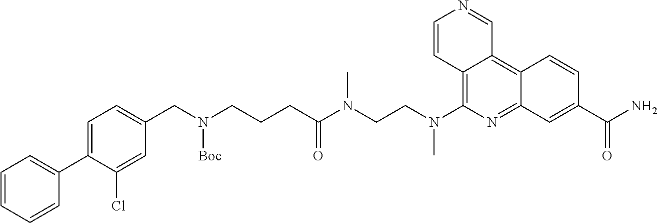 [SMILES: CN(CCN(C)c1nc2cc(C(N)=O)ccc2c2cnccc12)C(=O)CCCN(Cc1ccc(-c2ccccc2)c(Cl)c1)C(=O)OC(C)(C)C]

To a solution of Intermediate M (99.27 mg, 245.79 μmol) in THF (10 mL) was added DIPEA (63.53 mg, 491.58 μmol) followed by HATU (112.15 mg, 294.95 μmol) and compound 1.210 (85 mg, 245.79 μmol, HCl salt), sequentially at 25° C. The reaction mixture was stirred at 25° C. for 12 h. The mixture was poured into H₂O (50 mL) and extracted with EA (50 mL×3). The combined organic phases were washed (brine, 50 mL), dried over Na₂SO₄ and concentrated in vacuo to give the residue which was purified (PM62) to afford compound 1.211 (150 mg, 185.35 μmol, 62.8% yield, TFA salt) as a yellow solid.

LCMS (AM4): rt=0.867 min, (695.0 [M+H]⁺), 81.5% purity.

### Synthesis of Intermediate 1.218

### tert-Butyl (2-(4-formyl-1H-pyrazol-1-yl)ethyl)carbamate 1.213

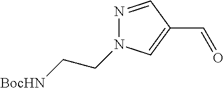 [SMILES: CC(C)(C)OC(=O)NCCn1cc(C=O)cn1]

To a solution of 1H-pyrazole-4-carbaldehyde (1 g, 7.54 mmol, HCl salt) in ACN (20 mL) was added tert-butyl N-(2-bromoethyl)carbamate (2.54 g, 11.32 mmol) and Cs₂CO₃ (4.92 g, 15.09 mmol) at RT. The mixture was then heated to 80° C. and stirred for 12 h. The reaction mixture was filtered and the filtrate was concentrated in vacuo. The residue was purified (PM2) to afford compound 1.213 (1.8 g, 7.52 mmol, 95.0% yield) as a colourless oil.

LCMS (AM3): rt=0.623 min, (140.1 [M−tBuCO₂+2H]⁺), 95.5% purity.

### (E)-tert-Butyl (2-(4-(2-nitrovinyl)-1H-pyrazol-1-yl)ethyl)carbamate 1.214

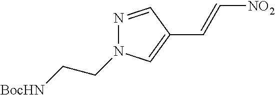 [SMILES: CC(C)(C)OC(=O)NCCn1cc(/C=C/[N+](=O)[O-])cn1]

To a solution of compound 1.213 (1.3 g, 5.43 mmol) in EtOH (10 mL) was added nitromethane (331.64 mg, 5.43 mmol) followed by AcOH (525.00 mg, 8.74 mmol) and piperidine (431.10 mg, 5.06 mmol), sequentially at 35° C. The reaction mixture was stirred at 35° C. for 12 h. The reaction mixture was poured into H₂O (50 mL) and extracted with EA (50 mL×2). The combined organic phases were washed (brine, 50 mL), dried (Na₂SO₄) and concentrated in vacuo to afford compound 1.214 (1 g, 3.54 mmol, 65.2% yield) as a yellow solid.

LCMS (AM3): rt=0.834 min, (283.1 [M+H]⁺), 88.1% purity.

### tert-Butyl (2-(4-(2-aminoethyl)-1H-pyrazol-1-yl)ethyl)carbamate 1.215

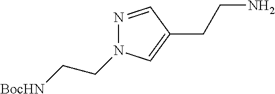 [SMILES: CC(C)(C)OC(=O)NCCn1cc(CCN)cn1]

To a solution of LAH (161.34 mg, 4.25 mmol) in THF (15 mL) was added compound 1.214 (400 mg, 1.42 mmol) at 20° C. The reaction mixture was stirred at 20° C. for 12 h. The reaction mixture was cooled to 0° C. and quenched by addition of H₂O (0.2 mL) and 10% NaOH aq. (0.2 mL) slowly. After stirring for 0.5 h, Na₂SO₄ (15 g) was added and the resulting suspension was stirred for 0.5 h and then filtered. The filtrate was concentrated in vacuo and the residue was purified (PM63) to afford compound 1.215 (170 mg, 461.52 μmol, 32.6% yield, TFA salt) as a colourless oil.

### Methyl 5-((2-(1-(2-((tert-butoxycarbonyl)amino)ethyl)-1H-pyrazol-4-yl)ethyl)amino)benzo[c][2,6]naphthyridine-8-carboxylate 1.216

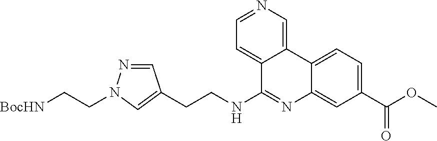 [SMILES: COC(=O)c1ccc2c(c1)nc(NCCc1cnn(CCNC(=O)OC(C)(C)C)c1)c1ccncc12]

To a solution of compound 1.1 (100 mg, 366.72 μmol) in DMSO (5 mL) was added DIPEA (94.79 mg, 733.44 μmol) and compound 1.215 (135.08 mg, 366.72 μmol) at RT. The reaction mixture was then heated to 80° C. and stirred for 12 h. The reaction mixture was poured into H₂O (50 mL) and extracted with EA (50 mL×2). The combined organic phases were washed (brine, 50 mL), dried (Na₂SO₄) and concentrated in vacuo. The residue was purified (PM40) to afford compound 1.216 (160 mg, 264.65 μmol, 60.1% yield, TFA salt) as a yellow solid.

LCMS (AM3): rt=0.807 min, (491.3 [M+H]⁺), 97.6% purity.

### Methyl 5-((2-(1-(2-aminoethyl)-1H-pyrazol-4-yl)ethyl)amino)benzo[c][2,6]naphthyridine-8-carboxylate 1.217

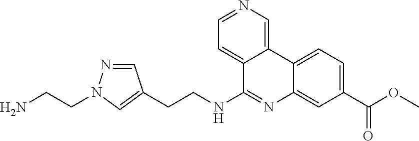 [SMILES: COC(=O)c1ccc2c(c1)nc(NCCc1cnn(CCN)c1)c1ccncc12]

To a solution of compound 1.216 (160 mg, 264.65 μmol) in DCM (2 mL) was added TFA (3.08 g, 27.01 mmol) at 30° C. The reaction mixture was stirred at 30° C. for 1 h. The reaction mixture was concentrated in vacuo to afford compound 1.217 (150 mg, TFA salt) as a colourless oil, which was used without purification.

LCMS (AM3): rt=0.668 min, (391.2 [M+H]⁺), 96.6% purity.

### Methyl 5-((2-(1-(2-(((2-chloro-[1,1′-biphenyl]-4-yl)methyl)amino)ethyl)-1H-pyrazol-4-yl)ethyl)amino)benzo[c][2,6]naphthyridine-8-carboxylate 1.218

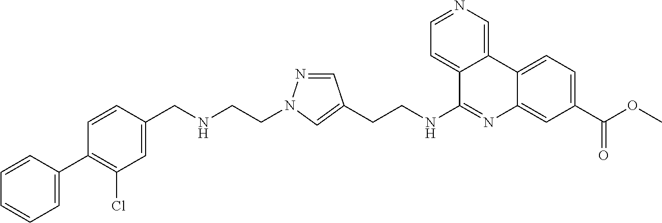 [SMILES: COC(=O)c1ccc2c(c1)nc(NCCc1cnn(CCNCc3ccc(-c4ccccc4)c(Cl)c3)c1)c1ccncc12]

To a solution of compound 1.217 (93.13 mg, 184.62 μmol) in MeOH (2 mL) was added 3-chloro-4-phenylbenzaldehyde (CAS 57592-44-6, 40 mg, 184.62 μmol) at 25° C. After stirring for 2 h, NaBH(OAc)₃ (117.38 mg, 553.86 μmol) was added, and the reaction mixture was stirred at 25° C. for 12 h. The reaction mixture was concentrated in vacuo and the residue was purified (PM64) to afford compound 1.218 (80 mg, 113.46 μmol, 41.0% yield, TFA salt) as a yellow oil.

LCMS (AM3): rt=0.823 min, (591.1 [M+H]⁺), 99.5% purity.

### Synthesis of Intermediate 1.220

### tert-Butyl (2-((8-carbamoylbenzo[c][2,6]naphthyridin-5-yl)amino)ethyl) (4-((3-chloro-4-(pyrazin-2-yl)benzyl)amino)butyl)carbamate 1.220

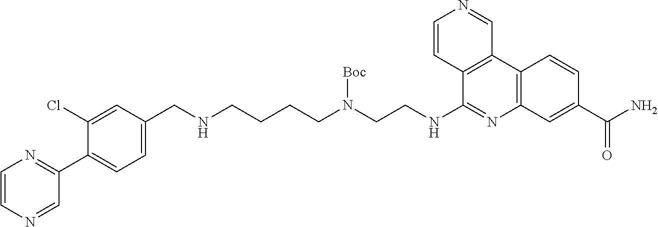 [SMILES: CC(C)(C)OC(=O)N(CCCCNCc1ccc(-c2cnccn2)c(Cl)c1)CCNc1nc2cc(C(N)=O)ccc2c2cnccc12]

To a mixture of compound 1.104 (100 mg, 0.135 mmol, TFA salt) in 1,4-dioxane (8 mL) and H₂O (2 mL) was added K₂CO₃ (80 mg, 0.578 mmol) followed by 2-bromopyrazine (43 mg, 0.27 mmol) and Pd(dppf)Cl₂ (10 mg, 0.014 mmol) at RT. The reaction mixture was degassed and purged with N₂ (×3), then it was heated to 80° C. and stirred for 16 h. The reaction mixture was concentrated in vacuo and the residue was purified (PM40) to afford compound 1.220 (35 mg, 33.1% yield, TFA salt) as a yellow solid.

LCMS (AM5): rt=0.615 min, (655.2 [M+H]⁺), 98.5% purity.

### Synthesis of Intermediate 1.221

### tert-Butyl (4-((2-((8-carbamoylbenzo[c][2,6]naphthyridin-5-yl)amino)ethyl)amino)butyl)(3-chloro-4-(pyrimidin-5-yl)benzyl)carbamate 1.221

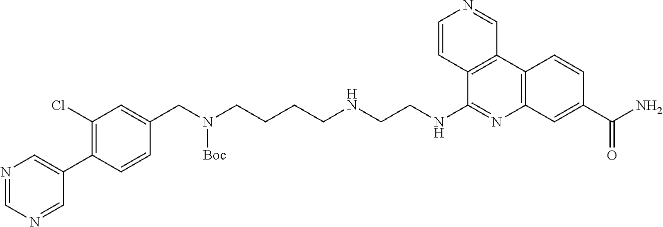 [SMILES: CC(C)(C)OC(=O)N(CCCCNCCNc1nc2cc(C(N)=O)ccc2c2cnccc12)Cc1ccc(-c2cncnc2)c(Cl)c1]

To a mixture of compound 1.200 (210 mg, 0.288 mmol, HCl salt) in 1,4-dioxane (10 mL) and H₂O (1 mL) was added K₂CO₃ (160 mg, 1.16 mmol) followed by pyrimidin-5-yl boronic acid (36 mg, 0.291 mmol) and Pd(PPh₃)₄ (33 mg, 0.029 mmol), sequentially at RT. The reaction mixture was degassed and purged with N₂ (×3), then heated to 80° C. and stirred for 3 h. The reaction mixture was poured onto H₂O (100 mL) and the resulting mixture was filtered. The filtrate was extracted with EA (50 mL×2) and the combined organic phases were washed (brine, 50 mL), dried (Na₂SO₄), filtered and concentrated in vacuo. The residue was purified (PM65) to afford compound 1.221 (58 mg, 28.2% yield, HCl salt) as a yellow solid.

LCMS (AM4): rt=0.713 min, (655.2 [M+H]⁺), 78.4% purity

### Synthesis of Intermediate 1.222

### tert-Butyl (4-((2-((8-carbamoylbenzo[c][2,6]naphthyridin-5-yl)amino)ethyl)amino)-4-oxobutyl)((2-chloro-[1,1′-biphenyl]-4-yl)methyl)carbamate 1.222

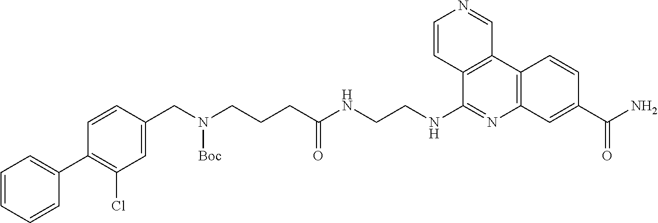 [SMILES: CC(C)(C)OC(=O)N(CCCC(=O)NCCNc1nc2cc(C(N)=O)ccc2c2cnccc12)Cc1ccc(-c2ccccc2)c(Cl)c1]

A mixture of Intermediate M (180 mg, 0.446 mmol), DIPEA (0.24 mL, 1.38 mmol) and HATU (200 mg, 0.526 mmol) in DMF (4 mL) was stirred at RT for 0.5 h, then Intermediate F (215 mg, 0.677 mmol, HCl salt) was added. The reaction mixture was stirred at RT for another 1.5 h. The reaction mixture was poured into H₂O (40 mL) and the resulting mixture was extracted with EA (20 mL×3). The combined organic phases were washed (brine, 40 mL), dried (Na₂SO₄), filtered and concentrated in vacuo. The residue was purified (PM24) to afford compound 1.222 (160 mg, 53.8% yield) as a light yellow solid.

LCMS (AM3): rt=0.828 min, (667.3 [M+H]⁺), 100% purity.

### Synthesis of Intermediate 1.227

### 3-((3-Chloro-4-(trifluoromethoxy)benzyl)amino)propanenitrile 1.223

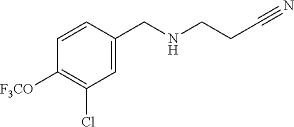 [SMILES: N#CCCNCc1ccc(OC(F)(F)F)c(Cl)c1]

A mixture of 3-chloro-4-(trifluoromethoxy)benzaldehyde (CAS 83279-39-4, 1 g, 4.45 mmol) and 3-aminopropanenitrile (780.32 mg, 11.13 mmol) in MeOH (20 mL) was stirred at 35° C. for 12 h, then NaBH(OAc)₃ (3.78 g, 17.81 mmol) was added. The reaction mixture was stirred at 35° C. for 3 h. The mixture was filtered and the filtrate was concentrated in vacuo. The residue was purified (PM46) to afford compound 1.223 (820 mg, 2.94 mmol, 66.1% yield) as a brown oil.

LCMS (AM3): rt=0.717 min, (278.9 [M+H]⁺), 100% purity.

### tert-Butyl 3-chloro-4-(trifluoromethoxy)benzyl(2-cyanoethyl)carbamate 1.224

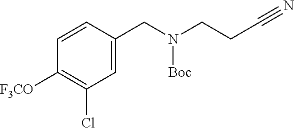 [SMILES: CC(C)(C)OC(=O)N(CCC#N)Cc1ccc(OC(F)(F)F)c(Cl)c1]

To a mixture of compound 1.223 (820 mg, 2.94 mmol) and TEA (893.31 mg, 8.83 mmol) in THF (10 mL) was added Boc₂O (706.45 mg, 3.24 mmol) at 20° C. The reaction mixture was stirred at 20° C. for 16 h. The mixture was concentrated in vacuo and the residue was purified (PM6) to afford compound 1.224 (1 g, 2.64 mmol, 89.7% yield) as a colourless oil.

LCMS (AM3): rt=1.207 min, (323.5 [M-tBu+2H]⁺), 91.3% purity.

### tert-Butyl 3-chloro-4-(trifluoromethoxy)benzyl(3-hydrazinyl-3-iminopropyl)carbamate 1.225

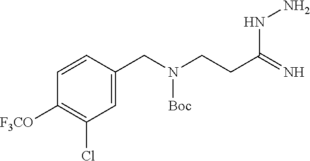 [SMILES: CC(C)(C)OC(=O)N(CCC(=N)NN)Cc1ccc(OC(F)(F)F)c(Cl)c1]

To a mixture of compound 1.224 (900 mg, 2.38 mmol) in a methanolic solution of NaOMe (2.45 M, 22.50 mL) was added hydrazine hydrate (1.55 g, 30.86 mmol) at RT. The reaction mixture was heated to 70° C. and stirred for 5 h. The mixture was adjusted to pH 7 by adding an aq. solution of FA (4 N). The mixture was filtered and the filtrate was concentrated in vacuo to give a residue that was purified (PM66) to afford compound 1.225 (170 mg, 413.81 μmol, 17.4% yield) as a colourless oil.

LCMS (AM3): rt=0.842 min, (411.1 [M+H]⁺), 79.8% purity.

### Methyl 5-((2-(5-(2-((tert-butoxycarbonyl)(3-chloro-4-(trifluoromethoxy)benzyl)amino)ethyl)-4H-1,2,4-triazol-3-yl)ethyl)amino)benzo[c][2,6]naphthyridine-8-carboxylate 1.226

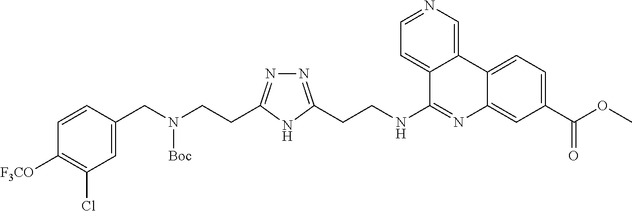 [SMILES: COC(=O)c1ccc2c(c1)nc(NCCc1nnc(CCN(Cc3ccc(OC(F)(F)F)c(Cl)c3)C(=O)OC(C)(C)C)[nH]1)c1ccncc12]

To a solution of Intermediate L (102.94 mg, 316.44 μmol) in DMF (2 mL) was added HATU (144.38 mg, 379.73 μmol) and DIPEA (122.69 mg, 949.33 μmol) at 30° C. The resulting mixture was stirred at 30° C. for 0.5 h and then compound 1.225 (130 mg, 316.44 μmol) was added. The reaction mixture was stirred at 30° C. for 4 h, and then heated to 110° C. and stirred for 16 h. The mixture was diluted with H₂O (20 mL) and the aq. phase was extracted with EA (20 mL×3). The combined organic phases were washed (brine, 50 mL), dried with Na₂SO₄, filtered and concentrated in vacuo. The residue was purified (PM67) to afford compound 1.226 (50 mg, 71.42 μmol, 22.6% yield) as a yellow solid.

LCMS (AM3): rt=0.933 min, (700.3 [M+H]⁺), purity: 96.7%.

### tert-Butyl (2-(5-(2-((8-carbamoylbenzo[c][2,6]naphthyridin-5-yl)amino)ethyl)-4H-1,2,4-triazol-3-yl)ethyl)(3-chloro-4-(trifluoromethoxy)benzyl)carbamate 1.227

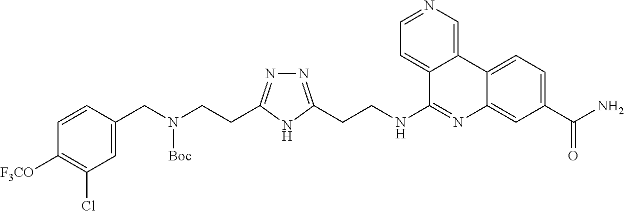 [SMILES: CC(C)(C)OC(=O)N(CCc1nnc(CCNc2nc3cc(C(N)=O)ccc3c3cnccc23)[nH]1)Cc1ccc(OC(F)(F)F)c(Cl)c1]

A mixture of compound 1.226 (50 mg, 71.42 μmol) in NH₃ in MeOH solution (7 M, 10 mL) was stirred at 80° C. for 40 h in a 30 ml sealed tube. The mixture was concentrated in vacuo to afford compound 1.227 (50 mg) as a white solid which was used without further purification.

LCMS (AM3): rt=0.880 min, (685.2 [M+H]⁺), purity: 59.9%.

### Synthesis of Intermediate 1.233

### N-(3-Chloro-4-(trifluoromethoxy)benzyl)-2-(1H-imidazol-4-yl) ethanamine 1.228

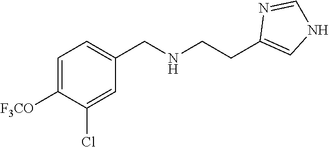 [SMILES: FC(F)(F)Oc1ccc(CNCCc2c[nH]cn2)cc1Cl]

To a mixture of 3-chloro-4-(trifluoromethoxy)benzaldehyde (CAS 83279-39-4, 2 g, 8.91 mmol) and 2-(1H-imidazol-4-yl) ethanamine (3.28 g, 17.81 mmol, 2× HCl salt) in DCE (50 mL) was added DIPEA (5.76 g, 44.53 mmol) and NaBH(OAc)₃ (7.55 g, 35.62 mmol), sequentially at 20° C. The reaction mixture was stirred at 20° C. for 12 h. The mixture was concentrated in vacuo and the residue was purified (PM61) to afford compound 1.228 (1.1 g, 3.44 mmol, 38.6% yield) as a yellow oil.

¹H NMR (400 MHz, CHCl₃-d) δ: 7.49 (s, 1H), 7.36 (d, J=1.6 Hz, 1H), 7.22-7.14 (m, 2H), 6.75 (s, 1H), 3.73 (s, 2H), 2.85 (t, J=6.4 Hz, 2H), 2.74 (t, J=6.4 Hz, 2H) ppm.

### tert-Butyl (2-(1H-imidazol-4-yl)ethyl)(3-chloro-4-(trifluoromethoxy)benzyl)carbamate 1.229

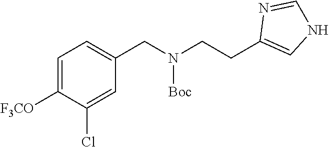 [SMILES: CC(C)(C)OC(=O)N(CCc1c[nH]cn1)Cc1ccc(OC(F)(F)F)c(Cl)c1]

To a mixture of compound 1.228 (900 mg, 2.82 mmol) in DCM (20 mL) was added Boc₂O (552.94 mg, 2.53 mmol) at 20° C. The reaction mixture was stirred at 20° C. for 2 h. The mixture was concentrated in vacuo and the residue was purified (PM61) to afford compound 1.229 (600 mg, 1.43 mmol, 50.8% yield) as a brown oil.

¹H NMR (400 MHz, CHCl₃-d) δ: 7.56 (s, 1H), 7.30-7.22 (m, 2H), 7.12 (d, J=7.8 Hz, 1H), 6.79 (br s, 1H), 4.33 (s, 2H), 3.67-3.40 (m, 2H), 2.95-2.80 (m, 2H), 1.46 (s, 9H) ppm.

### tert-Butyl 3-chloro-4-(trifluoromethoxy)benzyl(2-(1-(cyanomethyl)-1H-imidazol-4-yl)ethyl)carbamate 1.230

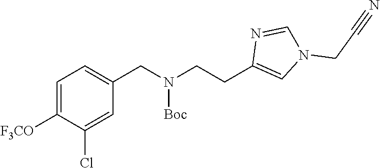 [SMILES: CC(C)(C)OC(=O)N(CCc1cn(CC#N)cn1)Cc1ccc(OC(F)(F)F)c(Cl)c1]

To a mixture of compound 1.229 (180 mg, 428.75 μmol) and 2-bromoacetonitrile (128.57 mg, 1.07 mmol) in ACN (4 mL) was added K₂CO₃ (177.77 mg, 1.29 mmol) at RT. The reaction mixture was heated to 80° C. and stirred for 16 h. The mixture was diluted with H₂O (30 mL) and the aq. phase was extracted with EA (30 mL×2). The combined organic phases were washed (brine, 50 mL), dried (Na₂SO₄), filtered and concentrated in vacuo. The residue was purified (PM3) to afford compound 1.230 (200 mg) as a black oil.

LCMS (AM3): rt=0.782 min, (459.2 [M+H]⁺), 91.9% purity.

### tert-Butyl (2-(1-(2-aminoethyl)-1H-imidazol-4-yl)ethyl)(3-chloro-4-(trifluoromethoxy)benzyl)carbamate 1.231

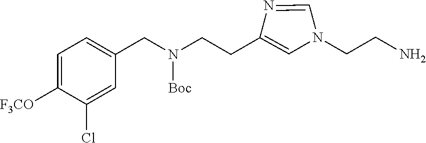 [SMILES: CC(C)(C)OC(=O)N(CCc1cn(CCN)cn1)Cc1ccc(OC(F)(F)F)c(Cl)c1]

To a solution of compound 1.230 (200 mg, 435.86 μmol) in MeOH (10 mL) was added Raney Ni (40.00 mg) and aq. ammonium hydroxide solution (728.00 mg, 6.23 mmol, 30% NH₃) under N₂ at 20° C. The suspension was degassed and purged with H₂ (×3). The reaction mixture was hydrogenated (15 psi) at 20° C. for 16 h. The mixture was filtered and the filtrate was concentrated in vacuo to afford compound 1.231 (200 mg) as a brown solid, which was used without purification.

LCMS (AM3): rt=0.803 min, (463.4 [M+H]⁺), purity: 76.1%.

### Methyl 5-((2-(4-(2-((tert-butoxycarbonyl)(3-chloro-4-(trifluoromethoxy)benzyl)amino)ethyl)-1H-imidazol-1-yl)ethyl)amino)benzo[c][2,6]naphthyridine-8-carboxylate 1.232

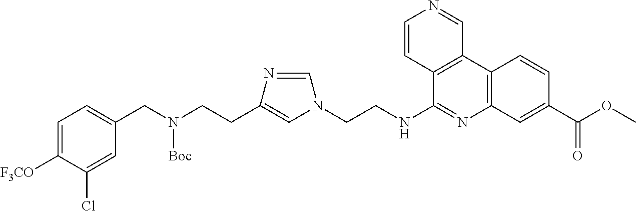 [SMILES: COC(=O)c1ccc2c(c1)nc(NCCn1cnc(CCN(Cc3ccc(OC(F)(F)F)c(Cl)c3)C(=O)OC(C)(C)C)c1)c1ccncc12]

To a mixture of compound 1.231 (200 mg, 432.07 μmol) and compound 1.1 (129.60 mg, 475.27 μmol) in DMSO (5 mL) was added DIPEA (167.52 mg, 1.30 mmol) at RT. The reaction mixture was heated to 80° C. and stirred for 12 h. The mixture was diluted with H₂O (30 mL) and extracted with EA (30 mL×2). The combined organic phases were washed (brine, 50 mL×2), dried (Na₂SO₄), filtered and concentrated in vacuo. The residue was purified (PM68) to afford compound 1.232 (110 mg, 157.34 μmol, 36.4% yield) as a yellow solid.

LCMS (AM3): rt=0.934 min, (699.0 [M+H]⁺), purity: 97.7%.

### tert-Butyl (2-(1-(2-((8-carbamoylbenzo[c][2,6]naphthyridin-5-yl)amino)ethyl)-1H-imidazol-4-yl)ethyl)(3-chloro-4-(trifluoromethoxy)benzyl)carbamate 1.233

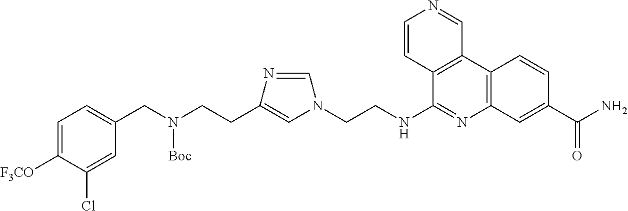 [SMILES: CC(C)(C)OC(=O)N(CCc1cn(CCNc2nc3cc(C(N)=O)ccc3c3cnccc23)cn1)Cc1ccc(OC(F)(F)F)c(Cl)c1]

A mixture of compound 1.232 (110 mg, 157.34 μmol) in a methanolic solution of ammonia (7 M, 10 mL) was stirred at 80° C. in a 30 mL sealed tube for 36 h. The mixture was concentrated in vacuo to afford compound 1.233 (110 mg) as a white solid, which was used without purification.

LCMS (AM3): rt=0.862 min, (684.2 [M+H]⁺), purity: 86.2%.

### Synthesis of Intermediate 1.234

### tert-Butyl (3-((3-((8-carbamoylbenzo[c][2,6]naphthyridin-5-yl)amino)propyl)amino)-3-oxopropyl)((2-chloro-[1,1′-biphenyl]-4-yl)methyl)carbamate 1.234

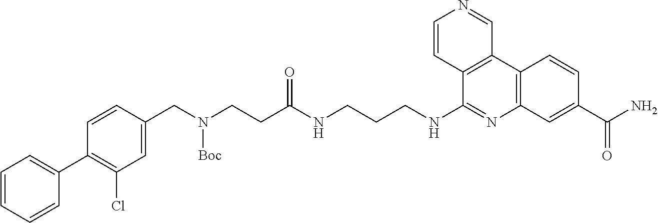 [SMILES: CC(C)(C)OC(=O)N(CCC(=O)NCCCNc1nc2cc(C(N)=O)ccc2c2cnccc12)Cc1ccc(-c2ccccc2)c(Cl)c1]

To a mixture of Intermediate D (117.50 mg, 301.39 μmol) and HATU (171.90 mg, 452.09 μmol) in DMF (5 mL) was added DIPEA (77.91 mg, 602.78 μmol) followed by compound 1.29 (100 mg, 301.39 μmol) at 25° C. The resulting mixture was stirred at 25° C. for 2 h. The mixture was poured into H₂O (50 mL) and extracted with EA (50 mL×2). The combined organic phases were washed (brine, 50 mL×3), dried (Na₂SO₄) and concentrated in vacuo to give compound 1.234 (150 mg) as a brown solid, which was used without further purification.

LCMS (AM3): rt=0.861 min, (667.4 [M+H]⁺), 50.9% purity.

### Synthesis of Intermediate 1.239

### Methyl 5-((2-((tert-butoxycarbonyl)amino)ethyl)(methyl)amino)benzo[c][2,6]naphthyridine-8-carboxylate 1.235

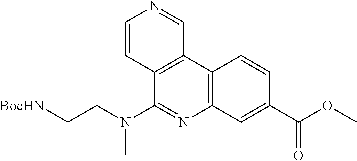 [SMILES: COC(=O)c1ccc2c(c1)nc(N(C)CCNC(=O)OC(C)(C)C)c1ccncc12]

A mixture of compound 1.1 (300 mg, 1.03 mmol), tert-butyl N-[2-(methylamino)ethyl] carbamate (269.20 mg, 1.55 mmol) and DIPEA (358.81 μL, 2.06 mmol) in DMSO (5 mL) was stirred at 70° C. for 15 h. The reaction mixture was poured into H₂O and the precipitate was collected by filtration to afford compound 1.235 (300 mg) as a brown solid, which was used without purification.

LCMS (AM3): rt=0.845 min, (411.2 [M+H]⁺), 98.4% purity.

### 5-((2-((tert-Butoxycarbonyl)amino)ethyl)(methyl)amino)benzo[c][2,6]naphthyridine-8-carboxylic acid 1.236

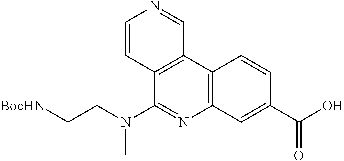 [SMILES: CN(CCNC(=O)OC(C)(C)C)c1nc2cc(C(=O)O)ccc2c2cnccc12]

To a mixture of compound 1.235 (300 mg, 730.88 μmol) in THF (5 mL), MeOH (5 mL) and H₂O (5 mL) was added LiOH·H₂O (61.34 mg, 1.46 mmol) at 20° C. The resulting mixture was stirred at 20° C. for 15 h. The solvent was removed in vacuo and the mixture was acidified with aq. 1 N HCl to pH 4. The formed precipitate was collected by filtration and dried under vacuum to afford compound 1.236 (250 mg) as a brown solid, which was used without purification.

LCMS (AM3): rt=0.777 min, (397.2 [M+H]⁺), 90.9% purity.

### tert-Butyl (2-((8-carbamoylbenzo[c][2,6]naphthyridin-5-yl)(methyl)amino)ethyl)carbamate 1.237

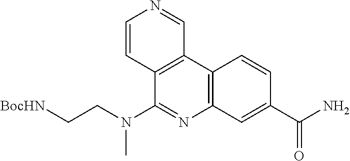 [SMILES: CN(CCNC(=O)OC(C)(C)C)c1nc2cc(C(N)=O)ccc2c2cnccc12]

To a mixture of compound 1.236 (250 mg, 630.61 μmol), EDCI (181.34 mg, 945.92 μmol), HOBt (127.82 mg, 945.92 μmol) and DIPEA (122.25 mg, 945.92 μmol) in DMF (5 mL) was added NH₄Cl (168.66 mg, 3.15 mmol) at 20° C. The resulting mixture was stirred at 20° C. for 15 h. The mixture was poured into H₂O (50 mL) and extracted with a mixed solution of DCM and MeOH (50 mL×2, 10:1). The combined organic phases were washed (brine, 50 mL), dried over Na₂SO₄ and concentrated to afford compound 1.237 (120 mg) as a brown solid, which was used without purification.

LCMS (AM3): rt=0.681 min, (396.2 [M+H]⁺), 83.8% purity.

### 5-((2-Aminoethyl)(methyl)amino)benzo[c][2,6]naphthyridine-8-carboxamide 1.238

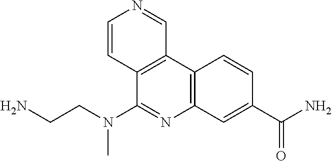 [SMILES: CN(CCN)c1nc2cc(C(N)=O)ccc2c2cnccc12]

To a solution of compound 1.237 (120 mg, 303.45 μmol) in 1,4-dioxane (5 mL) was added a solution of HCl in 1,4-dioxane (4 M, 3 mL) at 20° C. The resulting mixture was stirred at 20° C. for 1 h. The mixture was concentrated in vacuo and the residue was triturated with MTBE (10 mL), filtered and dried under reduced pressure to afford compound 1.238 (100 mg, HCl salt) as yellow solid.

LCMS (AM3): rt=0.234 min, (296.2 [M+H]⁺) 81.0% purity.

### tert-Butyl (4-((2-((8-carbamoylbenzo[c][2,6]naphthyridin-5-yl)(methyl)amino)ethyl)amino)-4-oxobutyl)((2-chloro-[1,1′-biphenyl]-4-yl)methyl)carbamate 1.239

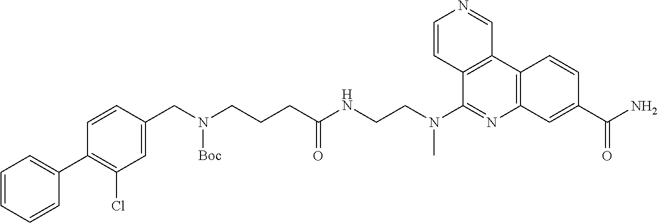 [SMILES: CN(CCNC(=O)CCCN(Cc1ccc(-c2ccccc2)c(Cl)c1)C(=O)OC(C)(C)C)c1nc2cc(C(N)=O)ccc2c2cnccc12]

To a solution of Intermediate M (121.73 mg, 301.39 μmol) and HATU (171.90 mg, 452.09 μmol) in DMF (5 mL) was added DIPEA (104.99 μL, 602.78 μmol) followed by compound 1.238 (100 mg, 301.39 μmol) at 20° C. The resulting mixture was stirred at 20° C. for 2 h. The mixture was poured into H₂O (50 mL) and extracted with EA (50 mL×2). The combined organic phases were washed (brine, 50 mL), dried (Na₂SO₄) and concentrated to afford compound 1.239 (150 mg) as a yellow solid that was used without further purification.

LCMS (AM3): rt=0.909 min, (681.4 [M+H]⁺), 44.0% purity.

### Synthesis of Intermediate 1.241

### 2-Chloro-2′-hydroxy-[1,1′-biphenyl]-4-carbaldehyde 1.240

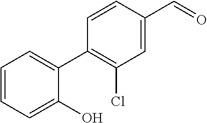 [SMILES: O=Cc1ccc(-c2ccccc2O)c(Cl)c1]

To a mixture of 4-Bromo-3-chlorobenzaldehyde (500 mg, 2.28 mmol) and (2-hydroxyphenyl)boronic acid (345.67 mg, 2.51 mmol) in 1,4-dioxane (5 mL) and H₂O (1 mL) was added Pd(dppf)Cl₂ (166.70 mg, 227.83 μmol) and Na₂CO₃ (482.95 mg, 4.56 mmol), sequentially at RT. The reaction mixture was heated to 80° C. and stirred for 16 h under N₂. The mixture was concentrated in vacuo and the residue was purified (PM6) to afford compound 1.240 (350 mg, 1.50 mmol, 66.0% yield) as a purple solid.

LCMS (AM3): rt=0.880 min, (233.1 [M+H]⁺), 79.1% purity.

### 2-Chloro-2′-(2-hydroxyethoxy)-[1,1′-biphenyl]-4-carbaldehyde 1.241

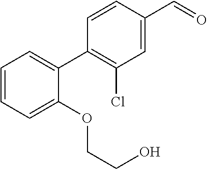 [SMILES: O=Cc1ccc(-c2ccccc2OCCO)c(Cl)c1]

To a mixture of 2-bromoethanol (177.25 mg, 1.42 mmol) and compound 1.240 (300 mg, 1.29 mmol) in DMF (8 mL) was added K₂CO₃ (356.41 mg, 2.58 mmol) at RT. The mixture was heated to 80° C. and stirred for 12 h. The reaction mixture was diluted with H₂O (30 mL) and extracted with EA (30 mL×2). The combined organic phases were washed (brine, 50 mL×3), dried (Na₂SO₄), filtered and concentrated in vacuo. The residue was purified (PM4) to afford compound 1.241 (200 mg, 722.77 μmol, 56.0% yield) as a yellow oil.

LCMS (AM3): rt=0.909 min, (277.5 [M+H]⁺), 90.1% purity.

### Synthesis of Intermediate 1.251

### 3-((3-Chloro-4-(trifluoromethoxy)benzyl)amino)propanoic acid 1.242

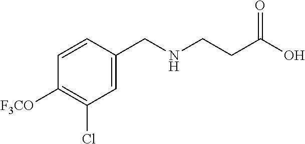 [SMILES: O=C(O)CCNCc1ccc(OC(F)(F)F)c(Cl)c1]

To a solution of 3-chloro-4-(trifluoromethoxy)benzaldehyde (CAS 83279-39-4, 7 g, 31.17 mmol) in MeOH (50 mL) was added 3-aminopropanoic acid (8.33 g, 93.51 mmol) at 20° C. After stirring for 24 h at 20° C., NaBH(OAc)₃ (19.82 g, 93.51 mmol) was added. The reaction mixture was stirred at 20° C. for 1 h. The mixture was filtered and the filtrate was concentrated in vacuo. The residue was purified (PM40) to afford compound 1.242 (7 g, 17.00 mmol, 54.5% yield, TFA salt) as a yellow oil.

LCMS (AM3): rt=0.703 min, (298.1 [M+H]⁺), 98.7% purity.

### 3-((tert-Butoxycarbonyl) (3-chloro-4-(trifluoromethoxy)benzyl)amino)propanoic acid 1.243

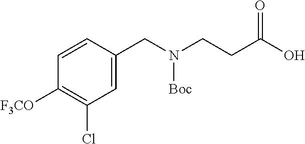 [SMILES: CC(C)(C)OC(=O)N(CCC(=O)O)Cc1ccc(OC(F)(F)F)c(Cl)c1]

To a solution of compound 1.242 (7 g, 23.52 mmol) in THF (50 mL) and H₂O (10 mL) was added NaHCO₃ (3.95 g, 47.03 mmol) and Boc₂O (7.70 g, 35.28 mmol) at 20° C. The reaction mixture was stirred at 20° C. for 12 h. The reaction mixture was acidified to pH 3 with aq. HCl (1 M) and then extracted with EA (50 mL×3). The combined organic layers were washed (brine, 20 mL), dried (Na₂SO₄), filtered and concentrated in vacuo. The residue was purified (PM2) to afford compound 1.243 (5.55 g, 13.95 mmol, 59.3% yield) as a yellow oil.

LCMS (AM3): rt=0.998 min, (420.0 [M+Na]⁺), 98.4% purity.

### Ethyl 4-(3-((tert-butoxycarbonyl)(3-chloro-4-(trifluoromethoxy)benzyl)amino)propanamido)-3-oxobutanoate 1.244

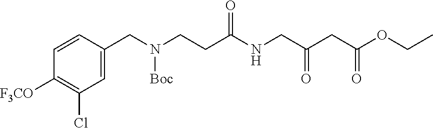 [SMILES: CCOC(=O)CC(=O)CNC(=O)CCN(Cc1ccc(OC(F)(F)F)c(Cl)c1)C(=O)OC(C)(C)C]

To a mixture of compound 1.243 (5.9 g, 14.83 mmol) and HATU (6.77 g, 17.80 mmol) in DMF (50 mL) was added DIPEA (5.75 g, 44.50 mmol) at 30° C. After stirring at 30° C. for 30 min, Ethyl 4-amino-3-oxobutanoate hydrochloride (2.69 g, 14.84 mmol, HCl salt) was added and the mixture was stirred at 30° C. for 2 h. The mixture was diluted with H₂O (100 mL) and the aq. phase was extracted with EA (100 mL×3). The combined organic phases were washed (brine, 200 mL), dried (Na₂SO₄), filtered and concentrated in vacuo. The residue was purified (PM46) to afford compound 1.244 (3.9 g, 7.43 mmol, 50.1% yield) as a red oil.

LCMS (AM3): rt=1.030 min, (525.1 [M+H]⁺), 74.0% purity.

### Ethyl 2-(2-(2-((tert-butoxycarbonyl)(3-chloro-4-(trifluoromethoxy)benzyl)amino)ethyl)oxazol-5-yl)acetate 1.245

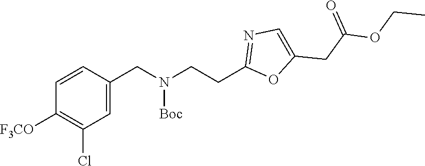 [SMILES: CCOC(=O)Cc1cnc(CCN(Cc2ccc(OC(F)(F)F)c(Cl)c2)C(=O)OC(C)(C)C)o1]

To a mixture of compound 1.244 (2.8 g, 5.33 mmol) in THF (40 mL) was added Burgess reagent (3.81 g, 16.00 mmol) at 20° C. The mixture was stirred at 20° C. for 2 h. The mixture was concentrated in vacuo and the residue was purified (PM4) to afford compound 1.245 (1.3 g, 2.56 mmol, 48% yield) as a yellow oil.

LCMS (AM3): rt=1.100 min, (507.2 [M+H]⁺), 94.0% purity.

### 2-(2-(2-((tert-Butoxycarbonyl)(3-chloro-4-(trifluoromethoxy)benzyl)amino)ethyl)oxazol-5-yl)acetic acid 1.246

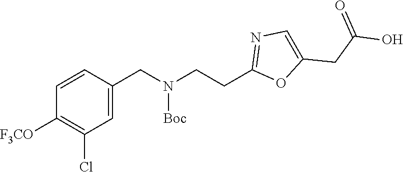 [SMILES: CC(C)(C)OC(=O)N(CCc1ncc(CC(=O)O)o1)Cc1ccc(OC(F)(F)F)c(Cl)c1]

To a mixture of compound 1.245 (1.1 g, 2.17 mmol) in THF (20 mL), MeOH (2 mL) and H₂O (2 mL) was added LiOH·H₂O (910.56 mg, 21.70 mmol) at 20° C. The mixture was stirred at 20° C. for 14 h. The mixture was acidified to pH 6 with aq. HCl (1 N) and the aq. phase was extracted with EA (30 mL×3). The combined organic phases were washed (brine, 80 mL), dried (Na₂SO₄), filtered and concentrated in vacuo to afford compound 1.246 (1.1 g) as a yellow oil, which was used without further purification.

LCMS (AM3): rt=1.002 min, (479.4 [M+H]⁺), 99.2% purity.

### tert-Butyl (2-(5-(2-amino-2-oxoethyl)oxazol-2-yl)ethyl)(3-chloro-4-(trifluoromethoxy)benzyl)carbamate 1.247

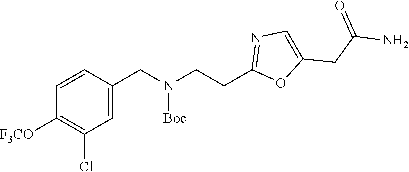 [SMILES: CC(C)(C)OC(=O)N(CCc1ncc(CC(N)=O)o1)Cc1ccc(OC(F)(F)F)c(Cl)c1]

To a mixture of compound 1.246 (500 mg, 1.04 mmol), EDCI (400.34 mg, 2.09 mmol) and HOBt (282.18 mg, 2.09 mmol) in DMF (5 mL) was added DIPEA (404.86 mg, 3.13 mmol) and NH₄Cl (223.42 mg, 4.18 mmol) at 30° C. The mixture was stirred at 30° C. for 12 h. The mixture was diluted with H₂O (30 mL) and the aq. phase was extracted with EA (30 mL×3). The combined organic phases were washed (brine, 80 mL×3), dried (Na₂SO₄), filtered and concentrated in vacuo to afford compound 1.247 (500 mg) as a yellow oil which was used without further purification.

### tert-Butyl 3-chloro-4-(trifluoromethoxy)benzyl(2-(5-(cyanomethyl)oxazol-2-yl)ethyl)carbamate 1.248

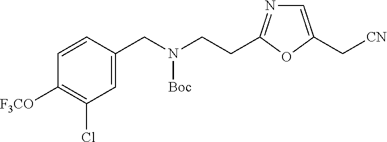 [SMILES: CC(C)(C)OC(=O)N(CCc1ncc(CC#N)o1)Cc1ccc(OC(F)(F)F)c(Cl)c1]

To a mixture of compound 1.247 (500 mg, 1.05 mmol) in THF (10 mL) was added Burgess reagent (500 mg, 2.10 mmol) at 20° C. The mixture was stirred at 20° C. for 1 h. The mixture was concentrated in vacuo and the residue was purified (PM3) to afford compound 1.248 (400 mg, 861.16 μmol, 82.3% yield) as a yellow oil.

LCMS (AM3): rt=0.948 min, (460.2 [M+H]⁺), purity: 99.2%.

### tert-Butyl (2-(5-(2-aminoethyl)oxazol-2-yl)ethyl)(3-chloro-4-(trifluoromethoxy)benzyl)carbamate 1.249

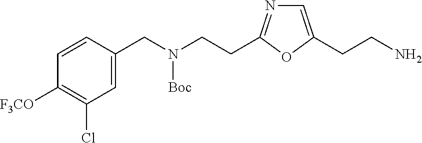 [SMILES: CC(C)(C)OC(=O)N(CCc1ncc(CCN)o1)Cc1ccc(OC(F)(F)F)c(Cl)c1]

To a solution of compound 1.248 (350 mg, 753.51 μmol) and aq. ammonium hydroxide (2.73 g, 23.37 mmol, 30%) in MeOH (15 mL) was added Raney-Ni (50 mg) under N₂ at 20° C. The suspension was degassed under vacuum and purged with hydrogen three times. The mixture was hydrogenated at 15 psi at 20° C. for 16 h. The mixture was filtered and the filtrate was concentrated in vacuo to afford compound 1.249 (260 mg, 560.49 μmol, 74.4% yield) as a colourless oil, which was used without purification.

LCMS (AM3): rt=0.862 min, (464.4 [M+H]⁺), purity: 95.5%.

### Methyl 5-((2-(2-(2-((tert-butoxycarbonyl)(3-chloro-4-(trifluoromethoxy)benzyl)amino)ethyl)oxazol-5-yl)ethyl)amino)benzo[c][2,6]naphthyridine-8-carboxylate 1.250

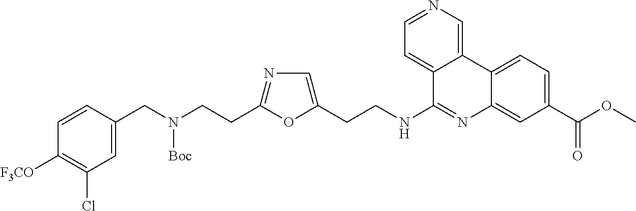 [SMILES: COC(=O)c1ccc2c(c1)nc(NCCc1cnc(CCN(Cc3ccc(OC(F)(F)F)c(Cl)c3)C(=O)OC(C)(C)C)o1)c1ccncc12]

To a mixture of compound 1.249 (260 mg, 560.49 μmol) and compound 1.1 (173.68 mg, 560.49 μmol) in DMSO (2 mL) was added DIPEA (217.32 mg, 1.68 mmol) at RT. The mixture was then heated to 70° C. and stirred for 12 h. The mixture was diluted with H₂O (50 mL) and the aq. phase was extracted with EA (50 mL×3). The combined organic phases were washed (brine, 100 mL), dried (Na₂SO₄), filtered and concentrated in vacuo. The residue was purified (PM40) to afford compound 1.250 (350 mg, 499.93 μmol, 89.2% yield) as a yellow solid.

LCMS (AM3): rt=1.001 min, (700.4 [M+H]⁺), purity: 93.3%.

### tert-Butyl (2-(5-(2-((8-carbamoylbenzo[c][2,6]naphthyridin-5-yl)amino)ethyl)oxazol-2-yl)ethyl)(3-chloro-4-(trifluoromethoxy)benzyl)carbamate 1.251

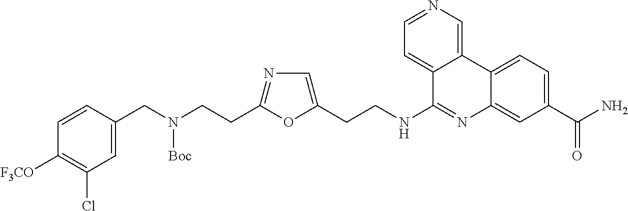 [SMILES: CC(C)(C)OC(=O)N(CCc1ncc(CCNc2nc3cc(C(N)=O)ccc3c3cnccc23)o1)Cc1ccc(OC(F)(F)F)c(Cl)c1]

A mixture of compound 1.250 (150 mg, 214.25 μmol) in saturated NH₃ in MeOH (7 M, 15 mL) was stirred at 80° C. in a 30 mL sealed tube for 16 h. The mixture was concentrated in vacuo to afford compound 1.251 (140 mg, 204.35 μmol, 95.4% yield) as a yellow solid which was used without purification.

LCMS (AM3): rt=0.901 min, (685.2 [M+H]⁺), purity: 62.6%.

### Synthesis of Intermediate 1.257

### Methyl 5-((2-((tert-butoxycarbonyl)(methyl)amino)ethyl)amino)benzo[c][2,6]naphthyridine-8-carboxylate 1.253

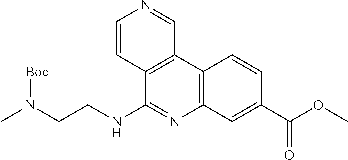

A mixture of compound 1.1 (300 mg, 1.03 mmol), tert-butyl N-(2-aminoethyl)-N-methyl-carbamate (270.29 mg, 1.55 mmol) and DIPEA (267.31 mg, 2.07 mmol) in DMSO (5 mL) was stirred at 70° C. for 15 h. The mixture was poured into H₂O and extracted with EA (100 mL×2). The combined organic phases were washed (brine, 50 mL), dried (Na₂SO₄) and concentrated to give compound 1.253 (420 mg) as a brown solid, which was used without further purification.

LCMS (AM3): rt=0.774 min, (411.2 [M+H]⁺), 67.1% purity.

### 5-((2-((tert-Butoxycarbonyl)(methyl)amino)ethyl)amino)benzo[c][2,6]naphthyridine-8-carboxylic acid 1.254

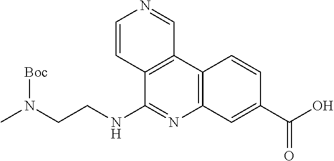 [SMILES: CN(CCNc1nc2cc(C(=O)O)ccc2c2cnccc12)C(=O)OC(C)(C)C]

To a solution of compound 1.253 (420 mg, 1.02 mmol) in THF (10 mL) and H₂O (5 mL) was added LiOH·H₂O (85.88 mg, 2.05 mmol) at 15° C. The resulting mixture was stirred at 15° C. for 15 h. The solvent was removed under reduced pressure and the residue was acidified with aq. 1 N HCl solution to pH 5. The precipitate was collected by filtration and dried under vacuum to afford compound 1.254 (350 mg) as yellow solid.

LCMS (AM3): rt=0.782 min, (397.1 [M+H]⁺), 92.5% purity.

### tert-Butyl (2-((8-carbamoylbenzo[c][2,6]naphthyridin-5-yl)amino)ethyl)(methyl)carbamate 1.255

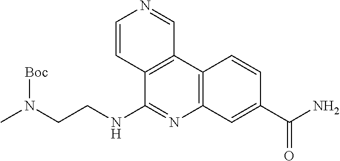 [SMILES: CN(CCNc1nc2cc(C(N)=O)ccc2c2cnccc12)C(=O)OC(C)(C)C]

To a solution of compound 1.254 (350 mg, 882.86 μmol), EDCI (253.87 mg, 1.32 mmol), HOBt (178.94 mg, 1.32 mmol) and DIPEA (228.21 mg, 1.77 mmol) in DMF (10 mL) was added NH₄Cl (141.68 mg, 2.65 mmol) at 15° C. The resulting mixture was stirred at 15° C. for 3 h. The mixture was poured into H₂O (50 mL) and extracted with EA (50 mL×2). The combined organic layer was washed (brine, 50 mL), dried (Na₂SO₄) and concentrated in vacuo to afford compound 1.255 (300 mg) as a yellow solid, which was used without further purification.

LCMS (AM3): rt=0.749 min, (396.2 [M+H]⁺), 60.8% purity.

### 5-((2-(Methylamino)ethyl)amino)benzo[c][2,6]naphthyridine-8-carboxamide 1.256

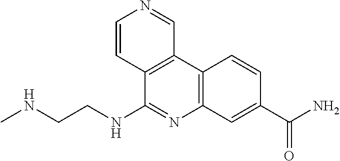 [SMILES: CNCCNc1nc2cc(C(N)=O)ccc2c2cnccc12]

To a solution of compound 1.255 (300 mg, 758.62 μmol) in 1,4-dioxane (5 mL) was added a solution of HCl in 1,4-dioxane (4 M, 5 mL) at 15° C. The resulting mixture was stirred at 15° C. for 15 h. The mixture was concentrated in vacuo and the residue was triturated with MTBE (50 mL) and filtered. The solid was dried under vacuum to afford compound 1.256 (220 mg, HCl salt) as a yellow solid.

LCMS (AM3): rt=0.265 min, (296.2 [M+H]⁺), 93.7% purity.

### tert-Butyl (4-((2-((8-carbamoylbenzo[c][2,6]naphthyridin-5-yl)amino)ethyl)(methyl)amino)-4-oxobutyl)((2-chloro-[1,1′-biphenyl]-4-yl)methyl)carbamate 1.257

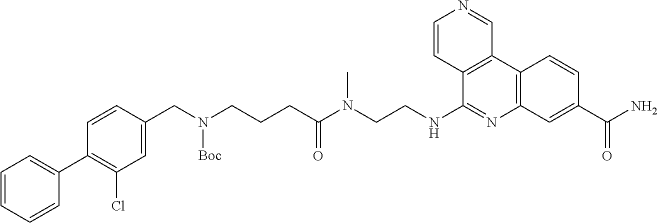 [SMILES: CN(CCNc1nc2cc(C(N)=O)ccc2c2cnccc12)C(=O)CCCN(Cc1ccc(-c2ccccc2)c(Cl)c1)C(=O)OC(C)(C)C]

To a solution of Intermediate M (100 mg, 247.59 μmol) and HATU (141.21 mg, 371.38 μmol) in DMF (5 mL) was added DIPEA (64.00 mg, 495.17 μmol) followed by compound 1.256 (98.58 mg, 297.10 μmol) at 15° C. The resulting mixture was stirred at 15° C. for 2 h. The mixture was poured into H₂O (50 mL) and extracted with EA (50 mL×2). The combined organic phases were washed (brine, 50 mL), dried (Na₂SO₄) and concentrated in vacuo to afford compound 1.257 (160 mg) as a yellow oil.

LCMS (AM3): rt=0.906 min, (681.3 [M+H]⁺), 86.7% purity.

### Synthesis of Intermediate 1.258

### tert-Butyl (2-((8-carbamoylbenzo[c][2,6]naphthyridin-5-yl)amino)ethyl) (4-(((5-phenylthiazol-2-yl)methyl)amino)butyl)carbamate 1.258

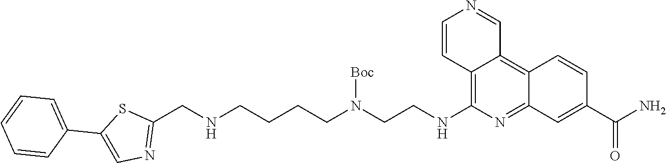 [SMILES: CC(C)(C)OC(=O)N(CCCCNCc1ncc(-c2ccccc2)s1)CCNc1nc2cc(C(N)=O)ccc2c2cnccc12]

To a solution of Intermediate G (100 mg, 220.97 μmol) in DCE (2 mL) was added 5-phenylthiazole-2-carbaldehyde (62.72 mg, 331.46 μmol) at 30° C. The mixture was stirred at 30° C. for 2 h and then NaBH(OAc)₃ (93.67 mg, 441.94 μmol) was added. The mixture was stirred at 30° C. for additional 12 h. The mixture was poured into H₂O and quenched with saturated NaHCO₃ aq. The aq. phase was extracted with a solvent mixture (EA:MeOH=20:1, 50 mL) and the organic layer was concentrated in vacuo to give compound 1.258 (40 mg) as a yellow oil, which was used without further purification.

### Synthesis of Intermediate 1.261

### Methyl 5-((3-aminopropyl)amino)benzo[c][2,6]naphthyridine-8-carboxylate 1.259

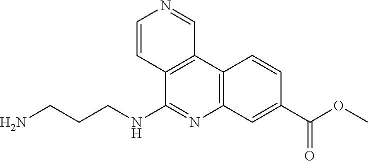 [SMILES: COC(=O)c1ccc2c(c1)nc(NCCCN)c1ccncc12]

To a solution of compound 1.26 (155 mg, 0.38 mmol) in 1,4-dioxane (5 mL) was added a solution of HCl in 1,4-dioxane (4 M, 15 mL) at 25° C. The mixture was stirred at 25° C. for 0.5 h. The mixture was concentrated in vacuo to give a residue which was purified (PM48) to afford compound 1.259 (100 mg, 0.28 mmol, 73.7% yield, HCl salt) as a yellow solid.

LCMS (AM1): rt=0.628 min, (311.1 [M+H]⁺), 97.2% purity.

### Methyl 5-((3-(3-((tert-butoxycarbonyl)(3-chloro-4-(trifluoromethoxy)benzyl)amino)propanamido)propyl)amino)benzo[c][2,6]naphthyridin e-8-carboxylate 1.260

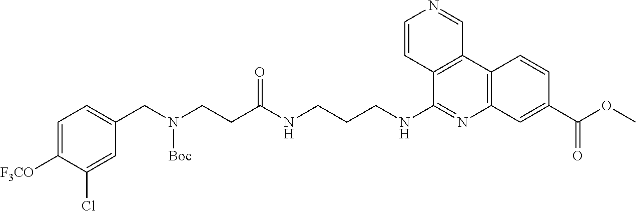 [SMILES: COC(=O)c1ccc2c(c1)nc(NCCCNC(=O)CCN(Cc1ccc(OC(F)(F)F)c(Cl)c1)C(=O)OC(C)(C)C)c1ccncc12]

To a solution of compound 1.243 (170 mg, 0.43 mmol) in DMF (5 mL) was added DIPEA (165 mg, 1.28 mmol) and HATU (211 mg, 0.55 mmol) at 35° C. After being stirred for 0.5 h, compound 1.259 (159 mg, 0.51 mmol) was added. The mixture was stirred at 35° C. for 11.5 h. The mixture was concentrated in vacuo to give a residue which was purified (PM48) to afford compound 1.260 (170 mg, 0.24 mmol, 57.1% yield) as a light yellow solid.

LCMS (AM3): rt=0.953 min, (690.3 [M+H]⁺), 99.2% purity.

### Methyl 5-((3-(3-((3-chloro-4-(trifluoromethoxy)benzyl)amino)propanamido)propyl)amino)benzo[c][2,6]naphthyridin e-8-carboxylate 1.261

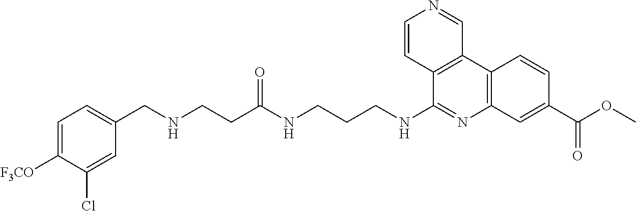 [SMILES: COC(=O)c1ccc2c(c1)nc(NCCCNC(=O)CCNCc1ccc(OC(F)(F)F)c(Cl)c1)c1ccncc12]

To a solution of compound 1.260 (140 mg, 202.8 μmol) in 1,4-dioxane (2 mL) was added a solution of HCl in 1,4-dioxane (4 M, 1 mL) at 25° C. The mixture was stirred at 25° C. for 0.5 h. The mixture was concentrated in vacuo to afford compound 1.261 (100 mg, 158.83 μmol, 78.3% yield, HCl salt) as a yellow solid, which was used without further purification.

LCMS (AM3): rt=0.613 min, (590.0 [M+H]⁺), 99.6% purity.

### Synthesis of Intermediate 1.268

### Ethyl 2-(2-(2-((tert-butoxycarbonyl) ((2-chloro-[1,1′-biphenyl]-4-yl)methyl)amino)ethyl)-1H-imidazol-5-yl)acetate 1.262

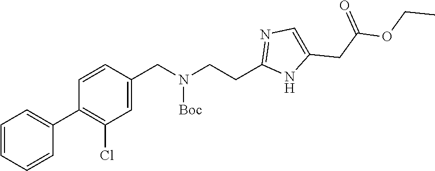 [SMILES: CCOC(=O)Cc1cnc(CCN(Cc2ccc(-c3ccccc3)c(Cl)c2)C(=O)OC(C)(C)C)[nH]1]

A mixture of compound 1.244 (2.9 g, 5.61 mmol) and NH₄OAc (4.32 g, 56.09 mmol) in toluene (60 mL) was stirred at reflux for 14 h. The reaction mixture was filtered and the filtrate was concentrated in vacuo. The residue was purified (PM40) to afford compound 1.262 (0.28 g) as a brown gum.

LCMS (AM3): rt=0.921 min, (498.3 [M+H]⁺), 74.9% purity.

### 2-(2-(2-((tert-Butoxycarbonyl)((2-chloro-[1,1′-biphenyl]-4-yl)methyl)amino)ethyl)-1H-imidazol-5-yl)acetic acid 1.263

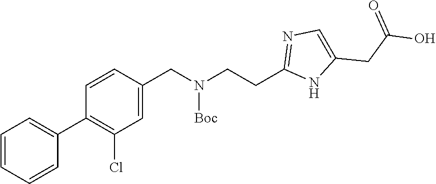 [SMILES: CC(C)(C)OC(=O)N(CCc1ncc(CC(=O)O)[nH]1)Cc1ccc(-c2ccccc2)c(Cl)c1]

To a mixture of compound 1.262 (0.28 g, 562.23 μmol) in THF (2 mL) and H₂O (1 mL) was added LiOH. H₂O (95 mg, 2.26 mmol) at 35° C. The resulting mixture was stirred at 35° C. for 2 h. The reaction mixture was poured onto H₂O (30 mL) and the aq. phase was adjusted to pH 5 with aq. HCl solution (1 N). The resulting mixture was extracted with EA (50 mL×2). The combined organic phases were washed (brine, 50 mL), dried (Na₂SO₄), filtered and concentrated in vacuo to afford compound 1.263 (260 mg) as a brown solid, which was used without further purification.

LCMS (AM3): rt=0.852 min, (470.3 [M+H]⁺), 84.2% purity.

### tert-Butyl (2-(5-(2-amino-2-oxoethyl)-1H-imidazol-2-yl)ethyl)((2-chloro-[1,1′-biphenyl]-4-yl)methyl)carbamate 1.264

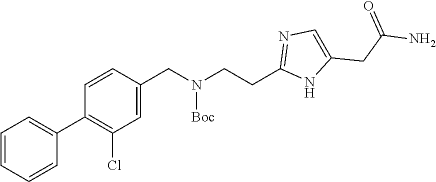 [SMILES: CC(C)(C)OC(=O)N(CCc1ncc(CC(N)=O)[nH]1)Cc1ccc(-c2ccccc2)c(Cl)c1]

To a mixture of compound 1.263 (260 mg, 553.24 μmol), NH₄Cl (177 mg, 3.31 mmol), HOBt (150 mg, 1.11 mmol) and DIPEA (0.385 mL) in DMF (4 mL) was added EDCI (212 mg, 1.11 mmol) at RT. The reaction mixture was heated to 70° C. and stirred for 2 h. The reaction mixture was filtered, and the filtrate was purified (PM48) to afford compound 1.264 (140 mg, 54% yield) as a brown solid.

LCMS (AM3): rt=0.850 min, (469.3 [M+H]⁺), 96.9% purity.

### tert-Butyl ((2-chloro-[1,1′-biphenyl]-4-yl)methyl)(2-(5-(cyanomethyl)-1H-imidazol-2-yl)ethyl)carbamate 1.265

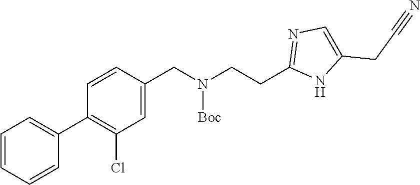 [SMILES: CC(C)(C)OC(=O)N(CCc1ncc(CC#N)[nH]1)Cc1ccc(-c2ccccc2)c(Cl)c1]

A mixture of compound 1.264 (130 mg, 0.277 mmol) and Burgess reagent (100 mg, 0.42 mmol) in THF (3 mL) was stirred at RT for 16 h. The mixture was poured into H₂O and extracted with EA (50 mL×2). The combined organic phases were washed (brine, 30 mL), dried (Na₂SO₄), filtered and concentrated in vacuo to afford compound 1.265 (100 mg) as a brown solid, which was used without further purification.

LCMS (AM3): rt=0.888 min, (451.3 [M+H]⁺), 67.9% purity.

### tert-Butyl (2-(5-(2-aminoethyl)-1H-imidazol-2-yl)ethyl)((2-chloro-[1,1′-biphenyl]-4-yl)methyl)carbamate 1.266

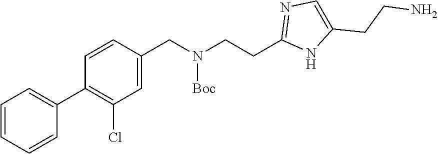 [SMILES: CC(C)(C)OC(=O)N(CCc1ncc(CCN)[nH]1)Cc1ccc(-c2ccccc2)c(Cl)c1]

A mixture of compound 1.265 (90 mg, 199.57 μmol), aq. ammonia (0.1 mL, 25%) and Raney-Ni (18 mg, 0.210 mmol) in MeOH (2 mL) was stirred under a H₂ atmosphere (15 psi) at RT for 23 h. The reaction mixture was filtered and the filtrate was concentrated in vacuo to afford compound 1.266 (120 mg) as a yellow solid, which was used without purification.

### Methyl 5-((2-(2-(2-((tert-butoxycarbonyl)((2-chloro-[1,1′-biphenyl]-4-yl)methyl)amino)ethyl)-1H-imidazol-5-yl)ethyl)amino)benzo[c][2,6]naphthyridine-8-carboxylate 1.267

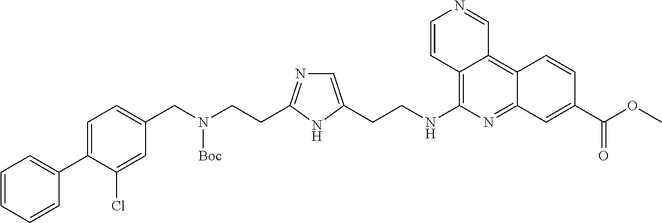 [SMILES: COC(=O)c1ccc2c(c1)nc(NCCc1cnc(CCN(Cc3ccc(-c4ccccc4)c(Cl)c3)C(=O)OC(C)(C)C)[nH]1)c1ccncc12]

A mixture of compound 1.266 (120 mg, 263.74 μmol), compound 1.1 (108 mg, 0.396 mmol) and DIPEA (0.115 mL, 660.24 μmol) in DMSO (3 mL) was stirred at 80° C. for 17 h. H₂O (20 mL) was added to the reaction mixture and the mixture was extracted with EA (20 mL×3). The combined organic phases were washed (brine, 20 mL), dried (Na₂SO₄), filtered and concentrated in vacuo. The crude product was purified (PM70) to afford compound 1.267 (70 mg) as a yellow solid.

LCMS (AM3): rt=0.912 min, (691.4 [M+H]⁺), 69.7% purity.

### tert-Butyl (2-(5-(2-((8-carbamoylbenzo[c][2,6]naphthyridin-5-yl)amino)ethyl)-1H-imidazol-2-yl)ethyl)((2-chloro-[1,1′-biphenyl]-4-yl)methyl)carbamate 1.268

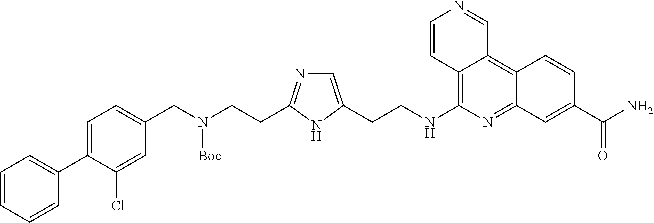 [SMILES: CC(C)(C)OC(=O)N(CCc1ncc(CCNc2nc3cc(C(N)=O)ccc3c3cnccc23)[nH]1)Cc1ccc(-c2ccccc2)c(Cl)c1]

A solution of compound 1.267 (70 mg, 101.27 μmol) in saturated NH₃ in MeOH (10 mL, 7 M) was stirred in a 30 mL sealed tube at 90° C. for 48 h. The reaction mixture was concentrated in vacuo and the residue was purified (PM71) to afford compound 1.268 (30 mg, 43.8% yield) as a yellow solid.

LCMS (AM3): rt=0.860 min, (676.3 [M+H]⁺), 100% purity.

### Synthesis of Intermediate 1.325

### tert-Butyl 3-chloro-4-(trifluoromethoxy)benzyl(3-((3,3-diethoxypropyl)amino)-3-oxopropyl)carbamate 1.324

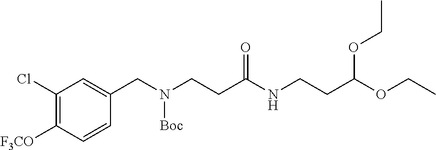 [SMILES: CCOC(CCNC(=O)CCN(Cc1ccc(OC(F)(F)F)c(Cl)c1)C(=O)OC(C)(C)C)OCC]

To a solution of compound 1.243 (1.3 g, 3.27 mmol), 3,3-diethoxypropan-1-amine (577.35 mg, 3.92 mmol) and TEA (1.4 mL, 10.06 mmol) in THF (30 mL) was added T₃P (50% in EA) (6.73 mmol, 4 mL) at 20° C. The resulting mixture was stirred at 20° C. for 1 h. The residue was poured into water (100 mL) and extracted with EA (50 mL×3). The combined organic phases were washed (brine, 50 mL×3), dried with anhydrous sodium sulfate, filtered and concentrated in vacuo. The residue was purified (PM2) to give compound 1.324 (933 mg, 1.77 mmol, 54.1% yield) as a yellow oil.

LCMS (AM3): rt=1.072 min, (549.3 [M+Na]⁺), 78.2% purity.

### tert-Butyl 3-chloro-4-(trifluoromethoxy)benzyl(3-oxo-3-((3-oxopropyl)amino)propyl)carbamate 1.325

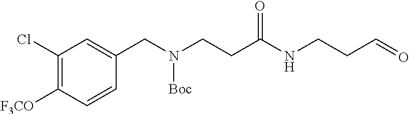 [SMILES: CC(C)(C)OC(=O)N(CCC(=O)NCCC=O)Cc1ccc(OC(F)(F)F)c(Cl)c1]

A mixture of compound 1.324 (533 mg, 1.01 mmol) in a solution of AcOH (6 mL, 104.91 mmol), THF (3 mL) and water (3 mL) was stirred at 20° C. for 1 h. The mixture was basified with saturated aq. sodium bicarbonate solution to pH=8 and then extracted with EA (20 mL×3). The combined organic phases were washed (brine, 30 mL×3), dried (Na₂SO₄), filtered and concentrated in vacuo to afford compound 1.325 (513 mg) as a yellow oil.

LCMS (AM3): rt=0.975 min, (453.1 [M+H]⁺), 82.9% purity.

### Synthesis of Intermediate 1.333

### Methyl 5-((2-cyanoethyl)amino)benzo[c][2,6]naphthyridine-8-carboxylate 1.329

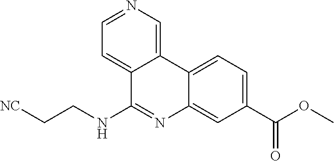 [SMILES: COC(=O)c1ccc2c(c1)nc(NCCC#N)c1ccncc12]

To a solution of compound 1.1 (300 mg, 1.10 mmol) and 3-aminopropanenitrile (80.97 mg, 1.16 mmol) in DMSO (10 mL) was added DIPEA (2.20 mmol, 383.25 μL) at RT. The mixture was heated to 80° C. and stirred for 12 h. MeOH (10 mL) was added and the precipitate was collected by filtration and dried under vacuum to give compound 1.329 (200 mg, 652.92 μmol, 54% yield) as a white solid.

LCMS (AM3): rt=0.777 min, (307.1 [M+H]⁺), 60.3% purity.

### Methyl 5-((3-(hydroxyamino)-3-iminopropyl)amino)benzo[c][2,6]naphthyridine-8-carboxylate 1.330

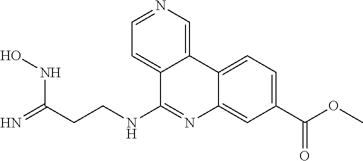 [SMILES: COC(=O)c1ccc2c(c1)nc(NCCC(=N)NO)c1ccncc12]

To a solution of compound 1.329 (120 mg, 391.75 μmol) in EtOH (10 mL) was added hydroxylamine hydrochloride (108.89 mg, 1.57 mmol) and sodium carbonate (166.08 mg, 1.57 mmol) at RT. The mixture was heated to 80° C. and stirred for 12 h. H₂O (5 mL) was added and the precipitate was collected by filtration and dried under vacuum to afford compound 1.330 (120 mg, 353.62 μmol, 72.2% yield) as a white solid.

LCMS (AM3): rt=0.672 min, (340.1 [M+H]⁺), 82.3% purity.

### Methyl 5-((2-(5-(2-((tert-butoxycarbonyl)((2-chloro-[1,1′-biphenyl]-4-yl)methyl)amino)ethyl)-1,2,4-oxadiazol-3-yl)ethyl)amino)benzo[c][2,6]naphthyridine-8-carboxylate 1.331

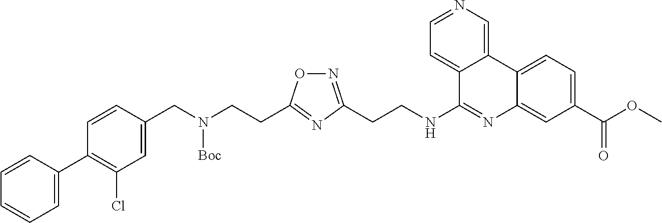 [SMILES: COC(=O)c1ccc2c(c1)nc(NCCc1noc(CCN(Cc3ccc(-c4ccccc4)c(Cl)c3)C(=O)OC(C)(C)C)n1)c1ccncc12]

To a mixture of compound 1.330 (100 mg, 294.68 μmol) in DMF (10 mL) was added Intermediate D (126.38 mg, 324.15 μmol), EDCI (67.79 mg, 353.62 μmol), HOBt (47.78 mg, 353.62 μmol) and DIPEA (102.66 μL, 589.37 μmol), sequentially at RT. The mixture was heated to 80° C. and stirred for 2 h. The reaction mixture was concentrated in vacuo and the residue was purified (PM46)) to give compound 1.331 (120 mg, 173.11 μmol, 49% yield) as a yellow solid.

LCMS (AM3): rt=0.973 min, (693.2 [M+H]⁺), 87.8% purity.

### 5-((2-(5-(2-((tert-Butoxycarbonyl)((2-chloro-[1,1′-biphenyl]-4-yl)methyl)amino)ethyl)-1,2,4-oxadiazol-3-yl)ethyl)amino)benzo[c][2,6]naphthyridine-8-carboxylic acid 1.332

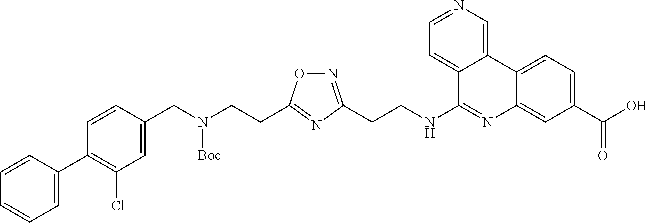 [SMILES: CC(C)(C)OC(=O)N(CCc1nc(CCNc2nc3cc(C(=O)O)ccc3c3cnccc23)no1)Cc1ccc(-c2ccccc2)c(Cl)c1]

To a solution of compound 1.331 (120 mg, 173.11 μmol) in THF (9 mL), MeOH (1 mL) and H₂O (1 mL), was added LiOH·H₂O (72.64 mg, 1.73 mmol) at 20° C. The reaction mixture was stirred at 20° C. for 12 h. The reaction mixture was acidified with aq. 1 M HCl to pH 4 and extracted with EA (10 mL×3). The combined organic layers were washed (brine, 10 mL), dried (Na₂SO₄), filtered and concentrated in vacuo to give compound 1.332 (100 mg, 147.24 μmol, 85% yield) as a yellow solid.

LCMS (AM3): rt=0.974 min, (679.3 [M+H]⁺), 90.9% purity.

### tert-Butyl (2-(3-(2-((8-carbamoylbenzo[c][2,6]naphthyridin-5-yl)amino)ethyl)-1,2,4-oxadiazol-5-yl)ethyl)((2-chloro-[1,1′-biphenyl]-4-yl)methyl)carbamate 1.333

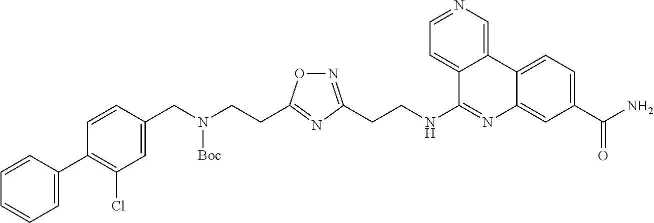 [SMILES: CC(C)(C)OC(=O)N(CCc1nc(CCNc2nc3cc(C(N)=O)ccc3c3cnccc23)no1)Cc1ccc(-c2ccccc2)c(Cl)c1]

To a solution of compound 1.332 (97.98 mg, 144.26 μmol) in THF (10 mL) was added HATU (65.82 mg, 173.11 μmol) and DIPEA (50.25 μL, 288.52 μmol) at 25° C. After being stirred for 10 min, NH₄Cl (8.49 mg, 158.69 μmol) was added. The mixture was stirred at 25° C. for 12 h. The mixture was poured into H₂O (50 mL) and extracted with EA (10 mL×3). The combined organic layers were washed (brine, 10 mL), dried (Na₂SO₄), filtered and concentrated to give compound 1.333 (100 mg) as a yellow solid.

LCMS (AM3): rt=0.916 min, (678.1 [M+H]⁺), 65.0% purity.

### Synthesis of Intermediate 1.335

### tert-Butyl (2-(N-(3-((8-carbamoylbenzo[c][2,6]naphthyridin-5-yl)amino)propyl)sulfamoyl)ethyl)carbamate 1.334

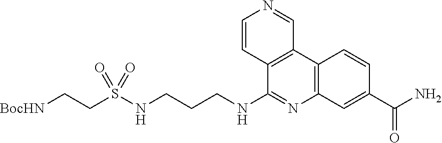 [SMILES: CC(C)(C)OC(=O)NCCS(=O)(=O)NCCCNc1nc2cc(C(N)=O)ccc2c2cnccc12]

To a solution of compound 1.29 (150 mg, 452.08 μmol) and DIPEA (157.49 μL, 904.16 μmol) in DMF (15 mL) was added tert-butyl (2-(chlorosulfonyl)ethyl)carbamate at 20° C. (132.21 mg, 542.50 μmol) (Protein and Peptide Letters, 2017, 24 (1), 56-63). The resulting mixture was stirred at 20° C. for 0.5 h. The mixture was poured into H₂O (50 mL) and extracted with EA (50 mL×2). The combined organic layers were washed (brine, 100 mL), dried (Na₂SO₄), filtered and concentrated in vacuo. The residue was purified (PM40) to give compound 1.334 (200 mg, 378.05 μmol, 83.6% yield) as a yellow solid.

LCMS (AM3): rt=0.645 min, (503.3 [M+H]⁺), 41.1% purity.

### 5-((3-(2-Aminoethylsulfonamido)propyl)amino)benzo[c][2,6]naphthyridine-8-carboxamide 1.335

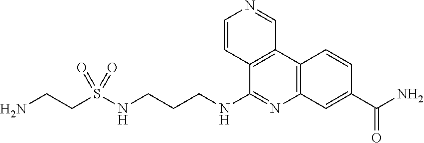 [SMILES: NCCS(=O)(=O)NCCCNc1nc2cc(C(N)=O)ccc2c2cnccc12]

To a solution of compound 1.334 (200 mg, 397.94 μmol) in DCM (20 mL) was added TFA (67.53 mmol, 5 mL) at 25° C. The mixture was stirred at 25° C. for 0.5 h. The mixture was concentrated in vacuo to afford compound 1.335 (200 mg, TFA salt) as a brown oil, which was used without purification.

LCMS (AM3): rt=0.202 min, (403.4 [M+H]⁺), 92.2% purity.

### Synthesis of Intermediate 1.338

### tert-Butyl (2-(((2-chloro-[1,1′-biphenyl]-4-yl)methyl)amino)ethyl)carbamate 1.336

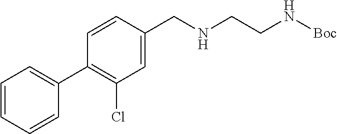 [SMILES: CC(C)(C)OC(=O)NCCNCc1ccc(-c2ccccc2)c(Cl)c1]

A solution of tert-butyl N-(2-aminoethyl)carbamate (2.96 g, 18.46 mmol) and 3-chloro-4-phenylbenzaldehyde (CAS 57592-44-6, 1 g, 4.62 mmol) in MeOH (30 mL) was stirred at 25° C. for 2 h, then NaBH(OAc)₃ (2.93 g, 13.85 mmol) was added. The mixture was stirred at 25° C. for 12 h. The mixture was concentrated to give a residue which was purified (PM46) to give compound 1.336 (1.1 g, 2.70 mmol, 58.6% yield, FA salt) as a white solid.

LCMS (AM3): rt=0.767 min, (361.2 [M+H]⁺), 95.0% purity.

### (9H-fluoren-9-yl)methyl (2-((tert-butoxycarbonyl)amino)ethyl)((2-chloro-[1,1′-biphenyl]-4-yl)methyl)carbamate 1.337

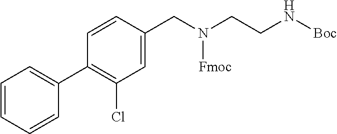

To a solution of compound 1.336 (1.1 g, 2.70 mmol) and DIPEA (941.73 μL, 5.41 mmol) in DCM (10 mL) was added (9H-fluoren-9-yl)methyl chloroformate (839.23 mg, 3.24 mmol) at 25° C. The mixture was stirred at 25° C. for 1 h. The reaction mixture was concentrated in vacuo and the residue purified (PM46) to afford compound 1.337 (1.4 g, 2.23 mmol, 79.9% yield) as a white solid.

LCMS (AM3): rt=1.113 min, (483.1 [M−tBuCO₂+2H]⁺), 96.7% purity

### (9H-fluoren-9-yl)methyl (2-aminoethyl)((2-chloro-[1,1′-biphenyl]-4-yl)methyl)carbamate 1.338

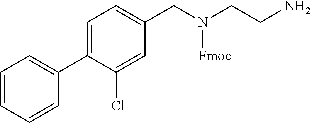

To a solution of compound 1.337 (1.4 g, 2.23 mmol) in 1,4-dioxane (6 mL) was added a solution of HCl in 1,4-dioxane (4 M, 6 mL) at 25° C. The mixture was stirred at 25° C. for 1 h. The mixture was concentrated in vacuo and the residue purified (PM61) to give compound 1.338 (1 g, 2.07 mmol, 93% yield) as a white solid.

LCMS (AM3): rt=0.842 min, (483.1 [M+H]⁺), 93.9% purity.

### Synthesis of Intermediate 1.344

### tert-Butyl (3-(((2-chloro-[1,1′-biphenyl]-4-yl)methyl)amino)propyl)carbamate 1.339

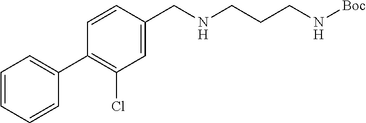 [SMILES: CC(C)(C)OC(=O)NCCCNCc1ccc(-c2ccccc2)c(Cl)c1]

A solution of tert-butyl N-(3-aminopropyl)carbamate (3.22 g, 18.48 mmol) and 3-chloro-4-phenylbenzaldehyde (CAS 57592-44-6, 1 g, 4.62 mmol) in MeOH (30 mL) was stirred at 25° C. for 2 h, then NaBH(OAc)₃ (2.93 g, 13.86 mmol) was added. The mixture was stirred at 25° C. for 12 h. The mixture was acidified with TFA (2 mL) and concentrated in vacuo to give compound 1.339 (2.2 g, TFA salt) as a yellow solid, which was used without purification.

### (9H-fluoren-9-yl)methyl (3-((tert-butoxycarbonyl)amino)propyl)((2-chloro-[1,1′-biphenyl]-4-yl)methyl)carbamate 1.340

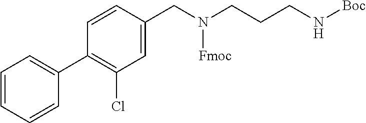

To a solution of compound 1.339 (2.2 g, 4.50 mmol) and DIPEA (13.50 mmol, 2.35 mL) in DCM (20 mL) was added (9H-fluoren-9-yl) methyl chloroformate (1.40 g, 5.40 mmol) at 25° C. The mixture was stirred at 25° C. for 1 h. The reaction mixture was concentrated in vacuo and the residue was purified (PM40) to afford compound 1.340 (2.6 g, 3.66 mmol, 81.3% yield) as a white solid.

LCMS (AM3): rt=1.217 min, (597.2 [M+H]⁺), 83.6% purity.

### (9H-fluoren-9-yl)methyl (3-aminopropyl)((2-chloro-[1,1′-biphenyl]-4-yl)methyl)carbamate 1.341

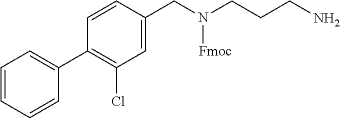

To a solution of compound 1.340 (2.6 g, 3.66 mmol) in 1,4-dioxane (20 mL) was added a solution of HCl in 1,4-dioxane (4 M, 10 mL) at 20° C. The reaction mixture was stirred at 20° C. for 1 h, then the mixture was concentrated in vacuo to give compound 1.341 (1.8 g, 3.62 mmol, 99% yield), which was used without purification.

### 9H-fluoren-9-ylmethyl N-[3-[2-(tert-butoxycarbonylamino)ethylsulfonylamino]propyl]-N-[(3-chloro-4-phenyl-phenyl)methyl]carbamate 1.342

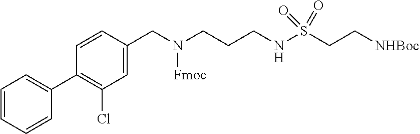

To a solution of compound 1.341 (1 g, 2.01 mmol) and DIPEA (4.02 mmol, 700.88 μL) in DMF (15 mL) was added tert-butyl (2-(chlorosulfonyl)ethyl)carbamate (588.40 mg, 2.41 mmol) at 15° C. The resulting mixture was stirred at 15° C. for 0.5 h. The mixture was poured into H₂O (100 mL) and extracted with EA (100 mL×2). The combined organic layers were washed (brine, 100 mL), dried (Na₂SO₄) and concentrated in vacuo. The residue was purified (PM3) to afford compound 1.342 (250 mg, 354.98 μmol, 17.6% yield) as a colourless oil.

LCMS (AM3): rt=1.158 min, (604.3 [M−tBuCO₂+2H]⁺), 23% purity.

### (9H-fluoren-9-yl)methyl (3-(2-aminoethylsulfonamido)propyl)((2-chloro-[1,1′-biphenyl]-4-yl)methyl)carbamate 1.343

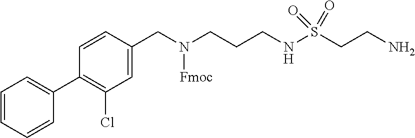

To a solution of compound 1.342 (250 mg, 354.98 μmol) in 1,4-dioxane (10 mL) was added a solution of HCl in 1,4-dioxane (4 M, 5 mL) at 25° C. The mixture was stirred at 25° C. for 1 h. The mixture was concentrated in vacuo and the residue was purified (PM74) to afford compound 1.343 (100 mg, 165.52 μmol, 46.6% yield) as a white solid.

LCMS (AM3): rt=0.910 min, (604.3 [M+H]⁺), 97.3% purity.

### Methyl 5-((2-(N-(3-(((2-chloro-[1,1′-biphenyl]-4-yl)methyl)amino)propyl) sulfamoyl)ethyl)amino)benzo[c][2,6]naphthyridine-8-carboxylate 1.344

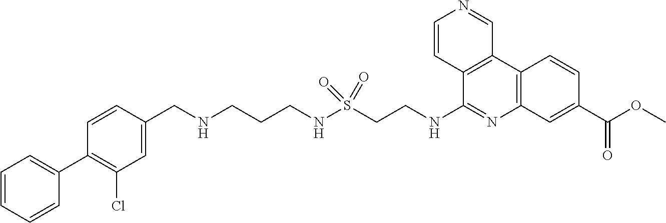 [SMILES: COC(=O)c1ccc2c(c1)nc(NCCS(=O)(=O)NCCCNCc1ccc(-c3ccccc3)c(Cl)c1)c1ccncc12]

A solution of compound 1.1 (40 mg, 146.69 μmol), compound 1.343 (93.05 mg, 154.02 μmol) and DIPEA (293.38 μmol, 51.10 μL) in DMSO (5 mL) was stirred at 80° C. for 12 h. The mixture was poured into H₂O (50 mL) and extracted with EA (50 mL×2). The combined organic phases were washed (brine, 50 mL), dried (Na₂SO₄) and concentrated in vacuo. The residue was purified (PM75) to afford compound 1.344 (100 mg, 136.58 μmol, 93.1% yield, TFA salt) as a yellow solid.

LCMS (AM3): rt=0.840 min, (618.0 [M+H]⁺), 95.5% purity

### Synthesis of Intermediate 1.347

### tert-Butyl (3-((3-((8-carbamoylbenzo[c][2,6]naphthyridin-5-yl)amino)propyl)(methyl)amino)-3-oxopropyl)carbamate 1.346

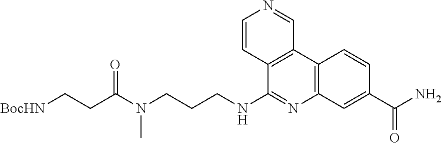 [SMILES: CN(CCCNc1nc2cc(C(N)=O)ccc2c2cnccc12)C(=O)CCNC(=O)OC(C)(C)C]

To a solution of 3-(tert-butoxycarbonylamino)propanoic acid (200 mg, 1.06 mmol) and HATU (439.79 mg, 1.16 mmol) in DMF (5 mL) was added DIPEA (1.16 mmol, 201.34 μL) at 20° C. After stirring for 0.5 h, compound 1.43 (200 mg, 578.33 μmol) was added. The mixture was stirred at 20° C. for 1.5 h. The mixture was poured into H₂O (50 mL) and extracted with EA (50 mL×2). The combined organic phases were washed (brine, 50 mL), dried (Na₂SO₄) and concentrated. The residue was purified (PM40) to afford compound 1.346 (200 mg, 378.73 μmol, 65.5% yield) as yellow solid.

LCMS (AM3): rt=0.727 min, (481.2 [M+H]⁺), 91.2% purity.

### 5-((3-(3-Amino-N-methylpropanamido)propyl)amino)benzo[c][2,6]naphthyridine-8-carboxamide 1.347

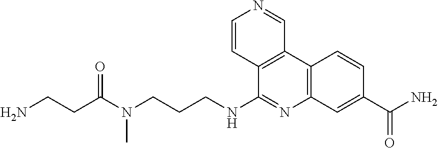 [SMILES: CN(CCCNc1nc2cc(C(N)=O)ccc2c2cnccc12)C(=O)CCN]

To a solution of compound 1.346 (200 mg, 378.73 μmol) in MeOH (2 mL) was added a solution of HCl in 1,4-dioxane (4 M, 2 mL) at 20° C. The resulting mixture was stirred at 20° C. for 3 h. The mixture was concentrated in vacuo and the residue was dried under vacuum to afford compound 1.347 (150 mg, HCl salt) as a yellow solid.

LCMS (AM3): rt=0.155 min, (381.5 [M+H]⁺), 98.7% purity.

### Synthesis of Intermediate 1.348

### Methyl 5-((2-(1-(2-((3-chloro-4-(trifluoromethoxy)benzyl)amino)ethyl)-1H-pyrazol-4-yl)ethyl)amino)benzo[c][2,6]naphthyridine-8-carboxylate 1.348

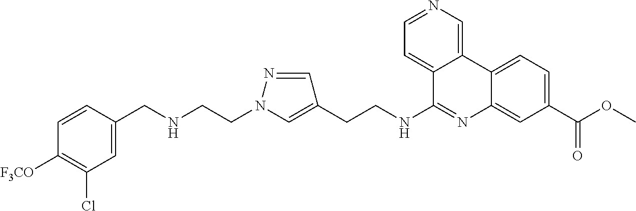 [SMILES: COC(=O)c1ccc2c(c1)nc(NCCc1cnn(CCNCc3ccc(OC(F)(F)F)c(Cl)c3)c1)c1ccncc12]

A solution of compound 1.217 (150 mg, 297.35 μmol), 3-chloro-4-(trifluoromethoxy)benzaldehyde (66.77 mg, 297.35 μmol) and DIPEA (594.69 μmol, 103.58 μL) in MeOH (6 mL) was stirred at 20° C. for 2 h, then NaBH₃CN (186.85 mg, 2.97 mmol) was added. The mixture was stirred at 20° C. for 12 h. The mixture was concentrated in vacuo to give a residue which was purified (PM76) to afford compound 1.348 (90 mg, 126.22 μmol, 42.5% yield, TFA salt) as yellow oil.

LCMS (AM3): rt=0.789 min, (599.0 [M+H]⁺), 29.4% purity.

### Synthesis of Intermediate 1.354

### N-((2-chloro-[1,1′-biphenyl]-4-yl)methyl)-2-(1H-imidazol-4-yl)ethanamine 1.349

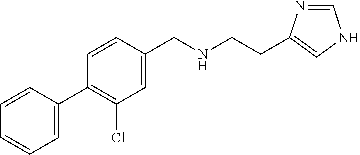 [SMILES: Clc1cc(CNCCc2c[nH]cn2)ccc1-c1ccccc1]

To a mixture of 2-(1H-imidazol-4-yl)ethanamine (3.40 g, 18.47 mmol, 2× HCl salt) in DCE (60 mL) was added DIPEA (45.93 mmol, 8 mL) followed by 3-chloro-4-phenylbenzaldehyde (CAS 57592-44-6, 2 g, 9.23 mmol) and NaBH(OAc)₃ (8.00 g, 37.75 mmol), sequentially at 35° C. The reaction mixture was stirred at 35° C. for 15 h. The reaction mixture was filtered and the filtrate was concentrated in vacuo. The residue was purified (PM48) to afford compound 1.349 (1.66 g, 45.8% yield, 2×HCl salt) as a white solid.

LCMS (AM3): rt=0.703 min, (312.0 [M+H]⁺), 98.4% purity.

### tert-Butyl (2-(1H-imidazol-4-yl)ethyl)((2-chloro-[1,1′-biphenyl]-4-yl)methyl)carbamate 1.350

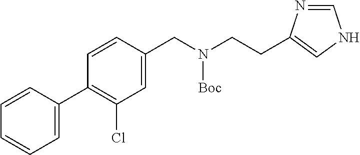 [SMILES: CC(C)(C)OC(=O)N(CCc1c[nH]cn1)Cc1ccc(-c2ccccc2)c(Cl)c1]

To a mixture of compound 1.349 (1.66 g, 4.31 mmol, 2×HCl salt) and TEA (2.50 mL, 17.99 mmol) in MeOH (60 mL) was added Boc₂O (0.99 mL, 4.31 mmol) at RT. The reaction mixture was stirred at RT for 20 h. The reaction mixture was concentrated in vacuo and the residue was purified (PM40) to afford compound 1.350 (1.46 g, 62.4% yield, TFA salt) as a colourless oil.

LCMS (AM3): rt=0.870 min, (412.1 [M+H]⁺), 97.1% purity.

### tert-Butyl ((2-chloro-[1,1′-biphenyl]-4-yl)methyl)(2-(1-(cyanomethyl)-1H-imidazol-4-yl)ethyl)carbamate 1.351

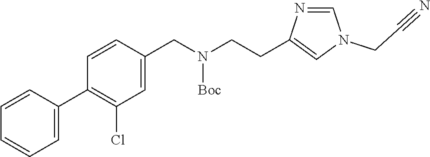 [SMILES: CC(C)(C)OC(=O)N(CCc1cn(CC#N)cn1)Cc1ccc(-c2ccccc2)c(Cl)c1]

A mixture of compound 1.350 (1.46 g, 2.78 mmol, TFA salt), 2-bromoacetonitrile (6.93 mmol, 0.462 mL) and K₂CO₃ (1.5 g, 10.85 mmol) in ACN (20 mL) was stirred under reflux for 15 h. The reaction mixture was filtered and the filtrate was concentrated in vacuo. The residue was purified (PM21) to afford compound 1.351 (1.05 g, 80.5% yield) as a dark brown gum.

LCMS (AM3): rt=0.885 min, (451.2 [M+H]⁺), 88.4% purity.

### tert-Butyl (2-(1-(2-aminoethyl)-1H-imidazol-4-yl)ethyl)((2-chloro-[1,1′-biphenyl]-4-yl)methyl)carbamate 1.352

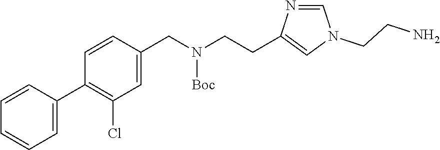 [SMILES: CC(C)(C)OC(=O)N(CCc1cn(CCN)cn1)Cc1ccc(-c2ccccc2)c(Cl)c1]

A mixture of compound 1.351 (1.05 g, 2.24 mmol), 25% aq. NH₃ solution (6.49 mmol, 1 mL) and Raney Ni (200 mg, 2.33 mmol) in MeOH (20 mL) was stirred under a H₂ balloon at RT for 23 h. The reaction mixture was filtered and the filtrate was concentrated in vacuo. The residue was purified (PM38) to afford compound 1.352 (180 mg, 0.316 mmol, 14% yield, TFA salt) as a yellow solid.

LCMS (AM3): rt=0.805 min, (455.2 [M+H]⁺), 100% purity.

### Methyl 5-((2-(4-(2-((tert-butoxycarbonyl)((2-chloro-[1,1′-biphenyl]-4-yl)methyl)amino)ethyl)-1H-imidazol-1-yl)ethyl)amino)benzo[c][2,6]naphthyridine-8-carboxylate 1.353

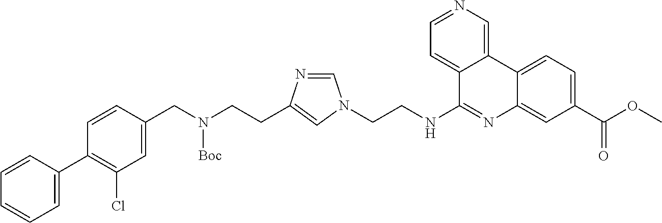 [SMILES: COC(=O)c1ccc2c(c1)nc(NCCn1cnc(CCN(Cc3ccc(-c4ccccc4)c(Cl)c3)C(=O)OC(C)(C)C)c1)c1ccncc12]

A mixture of compound 1.352 (180 mg, 0.316 mmol, TFA salt), compound 1.1 (86 mg, 0.315 mmol) and DIPEA (0.947 mmol, 0.165 mL) in DMSO (4 mL) was stirred at 80° C. for 17 h. The reaction mixture was added to H₂O (40 mL), the precipitate was collected by filtration and purified (PM77) to afford compound 1.353 (40 mg, 0.058 mmol, 18.3% yield) as a yellow solid.

LCMS (AM3): rt=0.935 min, (691.2 [M+H]⁺), 100% purity.

### tert-Butyl (2-(1-(2-((8-carbamoylbenzo[c][2,6]naphthyridin-5-yl)amino)ethyl)-1H-imidazol-4-yl)ethyl)((2-chloro-[1,1′-biphenyl]-4-yl)methyl)carbamate 1.354

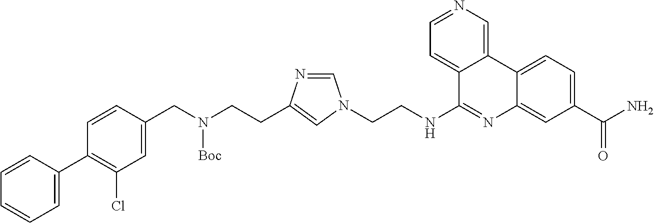 [SMILES: CC(C)(C)OC(=O)N(CCc1cn(CCNc2nc3cc(C(N)=O)ccc3c3cnccc23)cn1)Cc1ccc(-c2ccccc2)c(Cl)c1]

A mixture of compound 1.353 (40 mg, 0.058 mmol) in saturated NH₃ in MeOH (7M, 10 mL) was stirred in a 30 mL sealed tube at 80° C. for 20 h. The reaction mixture was concentrated in vacuo to afford compound 1.354 (40 mg) as a light yellow solid, which was used directly without further purification.

LCMS (AM3): rt=0.783 min, (676.4 [M+H]⁺), 54.9% purity.

### Synthesis of Intermediate 1.372

### tert-Butyl (4-(2-iodoethoxy)butyl)carbamate 1.372

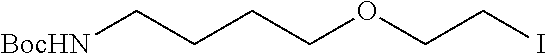 [SMILES: CC(C)(C)OC(=O)NCCCCOCCI]

To a mixture of compound 1.54 (1.3 g, 5.57 mmol), imidazole (569.03 mg, 8.36 mmol) and PPh₃ (2.19 g, 8.36 mmol) in DCM (10 mL) was added iodine (2.12 g, 8.36 mmol) at 0° C. under N₂. The mixture was then warmed to 25° C. and stirred for 12 h. The excess iodine was quenched with saturated aq. Na₂SO₃ solution (30 mL) and the mixture was extracted with EA (50 mL×3). The combined organic phase was washed (brine, 50 mL), dried (Na₂SO₄), filtered and concentrated in vacuo. The residue was purified (PM8) to afford compound 1.372 (1.4 g, 4.08 mmol, 73.2% yield) as a yellow oil.

¹H NMR (400 MHz, MeOH-d₄) δ: 3.70 (t, J=6.4 Hz, 2H), 3.52 (t, J=6.0 Hz, 2H), 3.31 (t, J=6.4 Hz, 2H), 3.08 (t, J=6.8 Hz, 2H), 1.65-1.54 (m, 4H), 1.45 (s, 9H) ppm.

### Synthesis of Intermediate 1.380

### (9H-fluoren-9-yl)methyl ((2-chloro-[1,1′-biphenyl]-4-yl)methyl)(2-(3-chloropropylsulfonamido)ethyl)carbamate 1.375

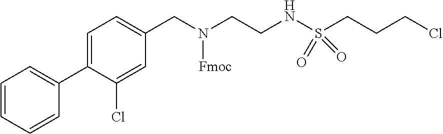

To a solution of compound 1.338 (800 mg, 1.66 mmol) in DMF (10 mL) was added TEA (3.31 mmol, 461.08 μL) and 3-chloropropane-1-sulfonyl chloride (351.90 mg, 1.99 mmol), and the reaction mixture was stirred at 25° C. for 1 h. The mixture was poured into H₂O (100 mL) and extracted with EA (50 mL×2). The combined organic phases were washed (brine, 50 mL), dried (Na₂SO₄) and concentrated in vacuo. The residue was purified (PM48) to give compound 1.375 (1 g, 1.60 mmol, 96.8% yield) as a white solid.

LCMS (AM3): rt=1.109 min, (623.1 [M+H]⁺), 90.5% purity.

### 3-Azido-N-(2-(((2-chloro-[1,1′-biphenyl]-4-yl)methyl)amino)ethyl)propane-1-sulfonamide 1.376

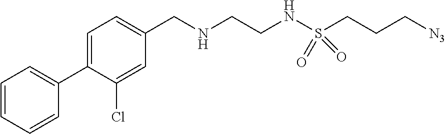 [SMILES: [N-]=[N+]=NCCCS(=O)(=O)NCCNCc1ccc(-c2ccccc2)c(Cl)c1]

To a solution of compound 1.375 (1 g, 1.60 mmol) in DMF (10 mL) was added sodium azide (521.26 mg, 8.02 mmol), and the resulting mixture heated to 50° C. and stirred for 3 h. The mixture was diluted with H₂O (100 mL) and extracted with EA (50 mL×2). The combined organic phases were washed (brine, 50 mL), dried over Na₂SO₄ and concentrated in vacuo. The residue was purified (PM46) to give compound 1.376 (500 mg, 1.10 mmol, 68.8% yield) as colourless oil.

LCMS (AM3): rt=0.788 min, (408.2 [M+H]⁺), 75.8% purity.

### tert-Butyl (2-(3-azidopropylsulfonamido)ethyl)((2-chloro-[1,1′-biphenyl]-4-yl)methyl)carbamate 1.377

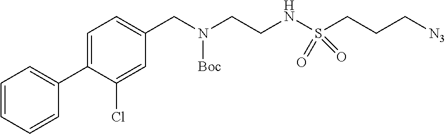 [SMILES: CC(C)(C)OC(=O)N(CCNS(=O)(=O)CCCN=[N+]=[N-])Cc1ccc(-c2ccccc2)c(Cl)c1]

To a solution of compound 1.376 (500 mg, 1.23 mmol) in DCM (10 mL) was added Boc₂O (321.02 mg, 1.47 mmol) and TEA (148.84 mg, 1.47 mmol), and the resulting mixture was stirred at 10° C. for 15 h. The mixture was concentrated in vacuo and the residue was purified (PM46) to afford compound 1.377 (400 mg, 747.98 μmol, 61% yield) as a colourless oil.

LCMS (AM3): rt=1.046 min, (408.1 [M−tBuCO₂+2H]⁺), 71.2% purity.

### tert-Butyl (2-(3-aminopropylsulfonamido)ethyl)((2-chloro-[1,1′-biphenyl]-4-yl)methyl)carbamate 1.378

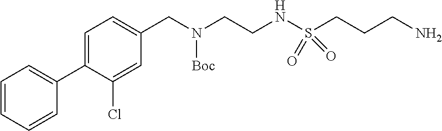 [SMILES: CC(C)(C)OC(=O)N(CCNS(=O)(=O)CCCN)Cc1ccc(-c2ccccc2)c(Cl)c1]

To a solution of compound 1.377 (400 mg, 747.98 μmol) in THF (5 mL) and H₂O (1 mL) was added PPh₃ (294.28 mg, 1.12 mmol), and the resulting mixture was stirred at 10° C. for 15 h. The mixture was concentrated and the residue was purified (PM2) to afford compound 1.378 (160 mg, 331.93 μmol, 44.4% yield) as a colourless oil.

LCMS (AM3): rt=0.841 min, (482.1 [M+H]⁺), 34.6% purity.

### Methyl 5-[3-[2-[tert-butoxycarbonyl-[(3-chloro-4-phenyl-phenyl)methyl]amino]ethylsulfamoyl]propylamino]benzo[c][2,6]naphthyridine-8-carboxylate 1.379

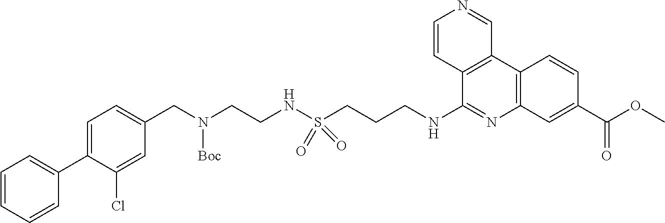 [SMILES: COC(=O)c1ccc2c(c1)nc(NCCCS(=O)(=O)NCCN(Cc1ccc(-c3ccccc3)c(Cl)c1)C(=O)OC(C)(C)C)c1ccncc12]

A mixture of compound 1.1 (64.28 mg, 207.45 μmol), compound 1.378 (100 mg, 207.45 μmol) and DIPEA (53.62 mg, 414.91 μmol) in DMSO (2 mL) was heated to 70° C. for 15 h. The mixture was poured into H₂O and extracted with EA (50 mL×2). The combined organic phases were washed (brine, 50 mL), dried (Na₂SO₄) and concentrated. The residue was purified (PM46) to afford compound 1.379 (50 mg, 69.61 μmol, 33.6% yield) as a yellow solid.

LCMS (AM3): rt=0.987 min, (718.4 [M+H]⁺), 52.9% purity.

### tert-Butyl (2-(3-((8-carbamoylbenzo[c][2,6]naphthyridin-5-yl)amino)propylsulfonamido)ethyl)((2-chloro-[1,1′-biphenyl]-4-yl)methyl)carbamate 1.380

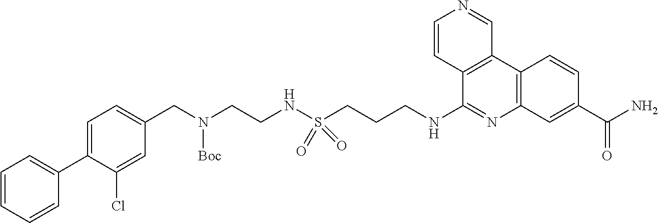 [SMILES: CC(C)(C)OC(=O)N(CCNS(=O)(=O)CCCNc1nc2cc(C(N)=O)ccc2c2cnccc12)Cc1ccc(-c2ccccc2)c(Cl)c1]

A mixture of compound 1.379 (40 mg, 55.69 μmol) in saturated NH₃ in MeOH (7 M, 10 mL) was heated to 80° C. and stirred for 15 h in a 30 mL sealed tube. The reaction was concentrated to afford compound 1.380 (35 mg, 49.77 μmol, 89.4% yield) as a yellow solid.

LCMS (AM3): rt=0.894 min, (703.2 [M+H]⁺), 62.0% purity.

### Synthesis of Intermediate 1.385

### tert-Butyl (4-((2-((8-(2H-tetrazol-5-yl)benzo[c][2,6]naphthyridin-5-yl)amino)ethyl)(tert-butoxycarbonyl)amino)butyl)((2-chloro-[1,1′-biphenyl]-4-yl)methyl)carbamate 1.385

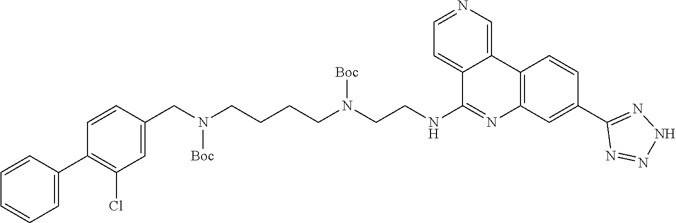 [SMILES: CC(C)(C)OC(=O)N(CCCCN(Cc1ccc(-c2ccccc2)c(Cl)c1)C(=O)OC(C)(C)C)CCNc1nc2cc(-c3nn[nH]n3)ccc2c2cnccc12]

To a mixture of compound 1.11 (95 mg, 0.122 mmol) and NH₄Cl (66 mg, 1.23 mmol) in DMF (10 mL) was added sodium azide (220 mg, 3.38 mmol), and the reaction mixture was heated to 100° C. and stirred for 23 h. The reaction mixture was diluted with H₂O (60 mL) and adjusted to pH 8 with saturated aq. NaHCO₃ solution. The resulting mixture was extracted with EA (30 mL×2), the combined organic phase was washed (brine, 30 mL), dried (Na₂SO₄), filtered and concentrated in vacuo. The residue was purified (PM191) to afford compound 1.385 (60 mg, 63% yield) as a yellow solid.

LCMS (AM1): rt=0.918 min, (778.3 [M+H]⁺), 100% purity.

### Synthesis of Intermediate 1.387

### Methyl 5-((3-(3-((tert-butoxycarbonyl)((2-chloro-[1,1′-biphenyl]-4-yl)methyl)amino)propanamido)propyl)amino)benzo[c][2,6]naphthyridine-8-carboxylate 1.386

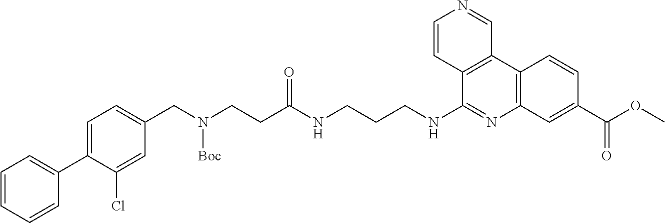 [SMILES: COC(=O)c1ccc2c(c1)nc(NCCCNC(=O)CCN(Cc1ccc(-c3ccccc3)c(Cl)c1)C(=O)OC(C)(C)C)c1ccncc12]

To a mixture of Intermediate D (300 mg, 0.769 mmol), compound 1.259 (370 mg, 1.07 mmol, HCl salt) and TEA (4.45 mmol, 0.62 mL) in DCM (10 mL) was added T₃P (1.51 mmol, 0.9 mL, 50% in EA) dropwise at 0° C. The reaction mixture was then warmed to RT and stirred for 15 h. The mixture was concentrated in vacuo and the residue was purified (PM40) to afford compound 1.386 (130 mg, 20.3% yield, TFA salt) as a light yellow solid.

LCMS (AM1): rt=0.837 min, (682.2 [M+H]⁺), 95.5% purity.

### 5-((3-(3-((tert-Butoxycarbonyl)((2-chloro-[1,1′-biphenyl]-4-yl)methyl)amino)propanamido)propyl)amino)benzo[c][2,6]naphthyridine-8-carboxylic acid 1.387

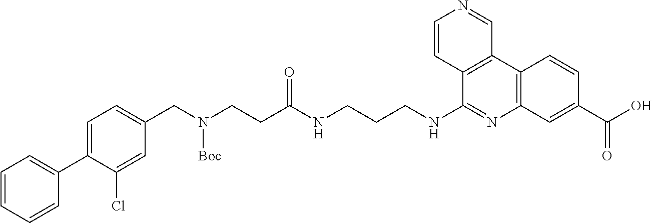 [SMILES: CC(C)(C)OC(=O)N(CCC(=O)NCCCNc1nc2cc(C(=O)O)ccc2c2cnccc12)Cc1ccc(-c2ccccc2)c(Cl)c1]

To a solution of compound 1.386 (120 mg, 0.176 mmol) in THF (5 mL), MeOH (5 mL) and H₂O (5 mL) was added LiOH·H₂O (14.76 mg, 0.352 mmol) at 20° C. The reaction mixture was stirred at 20° C. for 1 h. The reaction mixture was poured into H₂O (20 mL) and acidified to pH 5 with aq. HCl (1 M). The mixture was extracted with EA (20 mL×2), and the combined organic phases were washed (brine, 30 mL), dried (Na₂SO₄), filtered and concentrated in vacuo to afford compound 1.387 (120 mg, 97% yield) as light yellow solid, which was used directly without further purification.

LCMS (AM3): rt=0.918 min, (668.3 [M+H]⁺), 92.9% purity.

### Synthesis of Intermediate 1.388

### Methyl 5-((2-(4-((tert-butoxycarbonyl)((2-chloro-[1,1′-biphenyl]-4-yl)methyl)amino)butanamido)ethyl)amino)benzo[c][2,6]naphthyridine-8-carboxylate 1.388

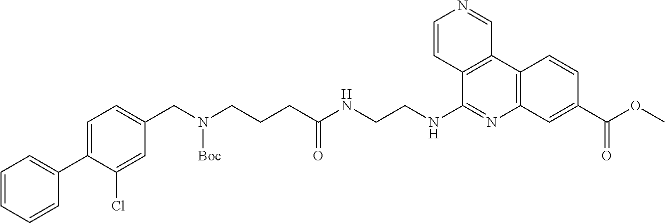 [SMILES: COC(=O)c1ccc2c(c1)nc(NCCNC(=O)CCCN(Cc1ccc(-c3ccccc3)c(Cl)c1)C(=O)OC(C)(C)C)c1ccncc12]

To a mixture of Intermediate M (340 mg, 0.842 mmol), Intermediate A (460 mg, 1.38 mmol, HCl salt) and TEA (5.03 mmol, 0.7 mL) in DCM (10 mL) was added T₃P (1.68 mmol, 1 mL, 50% in EA) dropwise at 0° C. The reaction mixture was warmed to RT and stirred for 2 h. The reaction mixture was diluted with saturated aq. NaHCO₃ solution (20 mL) and filtered through a pad of Celite®. The filter cake was washed with EA (20 mL×5) and the filtrate was concentrated in vacuo. The residue was purified (PM21) to afford compound 1.388 (220 mg) as a white solid.

LCMS (AM2): rt=0.933 min, (682.3 [M+H]⁺), 62.8% purity.

### Synthesis of Intermediate 1.393

### N-((2-chloro-[1,1′-biphenyl]-4-yl)methyl)-3,3-diethoxypropan-1-amine 1.389

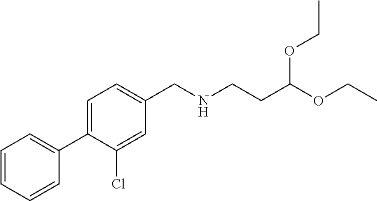 [SMILES: CCOC(CCNCc1ccc(-c2ccccc2)c(Cl)c1)OCC]

A mixture of 3-chloro-4-phenylbenzaldehyde (CAS 57592-44-6, 1 g, 4.20 mmol), 3,3-diethoxypropan-1-amine (800 mg, 5.43 mmol) and 4 Å MS (2 g, 4.20 mmol) in MeOH (20 mL) was stirred at 25° C. for 2 h, then NaBH₃CN (1.5 g, 23.87 mmol) was added. The mixture was stirred at 25° C. for 4 h. The mixture was poured into H₂O (50 mL) and extracted with EA (50 mL×3). The combined organic phases were washed (brine, 100 mL), dried (Na₂SO₄) and concentrated in vacuo to afford compound 1.389 (1.7 g), which was used directly without further purification.

LCMS (AM1): rt=0.714 min, (348.1 [M+H]⁺), 81.1% purity.

### tert-Butyl ((2-chloro-[1,1′-biphenyl]-4-yl)methyl)(3,3-diethoxypropyl)carbamate 1.390

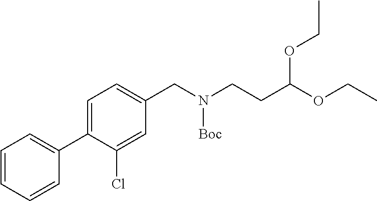 [SMILES: CCOC(CCN(Cc1ccc(-c2ccccc2)c(Cl)c1)C(=O)OC(C)(C)C)OCC]

To a mixture of compound 1.389 (0.5 g, 1.44 mmol) and NaHCO₃ (0.3 g, 3.57 mmol) in THF (20 mL) and H₂O (7 mL) was added (Boc)₂O (0.65 g, 2.98 mmol) at 25° C. The mixture was stirred at 25° C. for 5 h. The mixture was poured into H₂O (50 mL) and extracted with EA (50 mL×3). The combined organic phases were washed (brine, 50 mL), dried (Na₂SO₄) and concentrated in vacuo to afford compound 1.390 (0.9 g), which was used directly without further purification.

LCMS (AM1): rt=1.008 min, (470.2 [M+Na]⁺), 95.6% purity.

### tert-Butyl ((2-chloro-[1,1′-biphenyl]-4-yl)methyl) (3-oxopropyl)carbamate 1.391

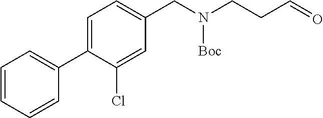 [SMILES: CC(C)(C)OC(=O)N(CCC=O)Cc1ccc(-c2ccccc2)c(Cl)c1]

A mixture of compound 1.390 (0.49 g, 1.09 mmol) in HOAc (5 mL), H₂O (5 mL) and THF (3 mL) was stirred at 25° C. for 40 h. The solution was slowly poured into saturated aq. NaHCO₃ solution (200 mL) and extracted with EA (50 mL×2). The combined organic phases were washed (brine, 100 mL), dried over Na₂SO₄ and concentrated in vacuo. The residue was purified (PM9) to afford compound 1.391 (220 mg, 544.89 μmol, 49.8% yield) as a colourless oil.

LCMS (AM3): rt=1.075 min, (318.2 [M-tBu+2H]⁺), 92.6% purity.

### Methyl 5-((2-((3-((tert-butoxycarbonyl) ((2-chloro-[1,1′-biphenyl]-4-yl)methyl)amino)propyl)amino)ethyl)amino)benzo[c][2,6]naphthyridine-8-carboxylate 1.392

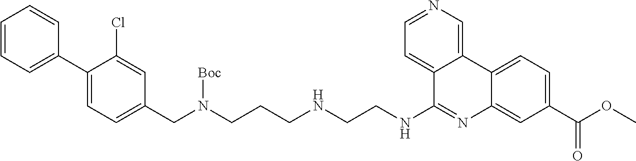 [SMILES: COC(=O)c1ccc2c(c1)nc(NCCNCCCN(Cc1ccc(-c3ccccc3)c(Cl)c1)C(=O)OC(C)(C)C)c1ccncc12]

A mixture of compound 1.391 (210 mg, 0.562 mmol), Intermediate A (205.61 mg, 0.618 mmol, HCl salt) and NaOAc (138.23 mg, 1.69 mmol) in DCE (10 mL) was stirred at 20° C. for 1 h, then NaBH(OAc)₃ (238.09 mg, 1.12 mmol) was added. The reaction mixture was stirred at 20° C. for 15 h. The reaction mixture was concentrated in vacuo and the residue was purified (PM46) to afford compound 1.392 (110 mg, 29.2% yield) as light yellow solid.

LCMS (AM3): rt=0.921 min, (654.2 [M+H]⁺), 97.4% purity.

### 5-((2-((3-((tert-Butoxycarbonyl)((2-chloro-[1,1′-biphenyl]-4-yl)methyl)amino)propyl)amino)ethyl)amino)benzo[c][2,6]naphthyridine-8-carboxylic acid 1.393

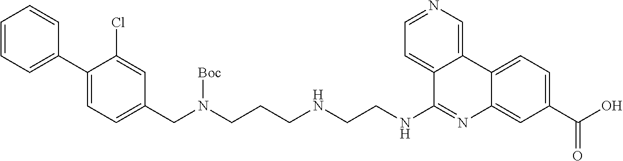 [SMILES: CC(C)(C)OC(=O)N(CCCNCCNc1nc2cc(C(=O)O)ccc2c2cnccc12)Cc1ccc(-c2ccccc2)c(Cl)c1]

To a mixture of compound 1.392 (110 mg, 0.164 mmol) in THF (2 mL), H₂O (2 mL) and MeOH (2 mL) was added LiOH·H₂O (14 mg, 0.33 mmol) at 25° C. The resulting mixture was stirred at 25° C. for 1 h. The reaction mixture was acidified with aq. HCl (1 M) to pH 5 and the resulting mixture was extracted with EA (20 mL×3). The combined organic phases were washed (brine, 50 mL), dried (Na₂SO₄), filtered and concentrated in vacuo to afford compound 1.393 (100 mg) as a light yellow solid, which was used directly without further purification.

LCMS (AM3): rt=0.869 min, (640.2 [M+H]⁺), 99.0% purity.

### Synthesis of Intermediate 1.420

### Methyl 5-cyclobutoxy-1H-indole-2-carboxylate 1.418

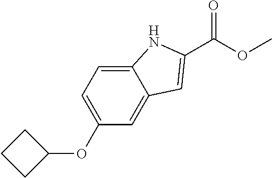 [SMILES: COC(=O)c1cc2cc(OC3CCC3)ccc2[nH]1]

A mixture of methyl 5-hydroxy-1H-indole-2-carboxylate (850 mg, 4.45 mmol) (Bioorganic and Medicinal Chemistry Letters, 2000, 10 (5), 483-486), bromocyclobutane (1.70 mL, 18.01 mmol) and K₂CO₃ (1.53 g, 11.07 mmol) in DMF (15 mL) was stirred at 80° C. for 20 h. The reaction mixture was poured into H₂O (45 mL), extracted with EA (20 mL×2), the combined organic phase washed (brine, 40 mL), dried (Na₂SO₄), filtered and concentrated in vacuo. The residue was purified (PM8) to afford compound 1.418 (700 mg, 60.3% yield) as a yellow solid.

LCMS (AM3): rt=0.942 min, (246.3 [M+H]⁺), 93.9% purity.

### (5-Cyclobutoxy-1H-indol-2-yl)methanol 1.419

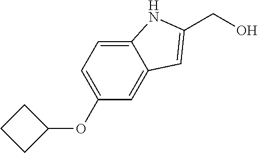 [SMILES: OCc1cc2cc(OC3CCC3)ccc2[nH]1]

To a mixture of LAH (260 mg, 6.85 mmol) in THF (20 mL) was added compound 1.418 (650 mg, 2.65 mmol) in THF (5 mL) at 0° C. The reaction mixture was warmed to room temperature, stirred for 2 h, then the reaction quenched with H₂O (0.5 mL) followed by aq. 10% NaOH solution (0.5 mL) and H₂O (1 mL). The reaction mixture was stirred for 1 h, then Na₂SO₄ added, the mixture filtered and the filtrate concentrated in vacuo to afford compound 1.419 (470 mg, 81.6% yield) as a white solid, which was used directly without purification.

LCMS (AM3): rt=0.840 min, (218.3 [M+H]⁺), 90.3% purity.

### 5-Cyclobutoxy-1H-indole-2-carbaldehyde 1.420

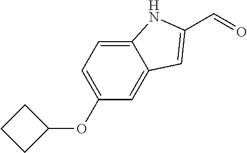 [SMILES: O=Cc1cc2cc(OC3CCC3)ccc2[nH]1]

A mixture of compound 1.419 (470 mg, 2.16 mmol) and MnO₂ (1.88 g, 21.63 mmol) in DCM (20 mL) was stirred at room temperature for 12 h. The reaction mixture was filtered and the filtrate was concentrated in vacuo. The residue was purified (PM8) to afford compound 1.420 (370 mg, 58.8% yield) as a brown solid.

LCMS (AM4): rt=0.846 min, (216.1 [M+H]⁺), 74.2% purity.

### Synthesis of Intermediate 1.427

### Methyl 1-(4-((tert-butoxycarbonyl)amino)butyl)-1H-1,2,3-triazole-4-carboxylate 1.422

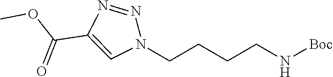 [SMILES: COC(=O)c1cn(CCCCNC(=O)OC(C)(C)C)nn1]

To a solution of tert-butyl (4-azidobutyl)carbamate, (CAS 129392-85-4, 11.3 g, 52.74 mmol) and CuI (2 g, 10.50 mmol) in DMF (100 mL) and MeOH (15 mL) was added methyl prop-2-ynoate (5 mL, 60.07 mmol). The reaction mixture was then heated to 100° C. and stirred for 2 h. The reaction mixture was poured into a mixture of H₂O:EA (v/v=3:1, 400 mL) and filtered. The filtrate was extracted with EA (100 mL×3). The combined organic phase was washed (brine, 200 mL×2), dried (Na₂SO₄), filtered and concentrated in vacuo. The residue was triturated with a mixture of MTBE:EtOH (v/v=2:1, 120 mL) and then filtered. The solid was dried in vacuum to afford compound 1.422 (11 g, 69.9% yield) as a white solid.

¹H NMR (400 MHz, MeOH-d₄) δ: 8.55 (s, 1H), 4.49 (t, J=7.2 Hz, 2H), 3.91 (s, 3H), 3.07 (t, J=6.8 Hz, 2H), 1.98-1.90 (quin, 2H), 1.50-1.44 (quin, 2H), 1.42 (s, 9H) ppm.

### tert-Butyl (4-(4-carbamoyl-1H-1,2,3-triazol-1-yl)butyl)carbamate 1.423

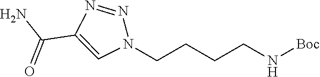 [SMILES: CC(C)(C)OC(=O)NCCCCn1cc(C(N)=O)nn1]

A mixture of compound 1.422 (11 g, 36.87 mmol) in NH₃/MeOH (7 M, 100 mL) was stirred at RT for 16 h. The reaction mixture was concentrated in vacuo to afford compound 1.423 (10.45 g, 100% yield) as a white solid, which was used directly without purification.

LCMS (AM3): rt=0.662 min, (306.1 [M+Na]⁺), 87.5% purity.

### tert-Butyl (4-(4-amino-1H-1,2,3-triazol-1-yl)butyl)carbamate 1.424

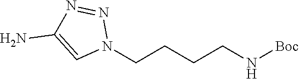 [SMILES: CC(C)(C)OC(=O)NCCCCn1cc(N)nn1]

To a solution of NaOH (4.07 g, 101.65 mmol) in H₂O (40 mL) was added Br₂ (1.31 mL, 25.41 mmol) at 0° C. The reaction mixture was stirred at 0° C. for 30 min before compound 1.423 (3.6 g, 12.71 mmol) was added. The reaction mixture was stirred at RT for 30 min, then heated to 80° C. and stirred for 2 h. The reaction mixture was neutralized by slow addition of conc. HCl and concentrated in vacuo. The residue was purified (PM39) to afford compound 1.424 (0.9 g, 24.1% yield) as a yellow solid.

LCMS (AM3): rt=0.688 min, (256.2 [M+H]⁺), 87% purity.

### tert-Butyl (4-(4-(((2-chloro-[1,1′-biphenyl]-4-yl)methyl)amino)-1H-1,2,3-triazol-1-yl)butyl)carbamate 1.425

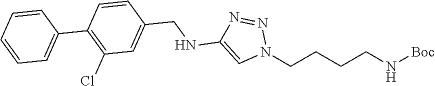 [SMILES: CC(C)(C)OC(=O)NCCCCn1cc(NCc2ccc(-c3ccccc3)c(Cl)c2)nn1]

A mixture of 3-chloro-4-phenylbenzaldehyde (CAS 57592-44-6, 0.25 g, 1.15 mmol), compound 1.424 (0.3 g, 1.18 mmol) and AcOH (2.34 mmol, 0.134 mL) in DCE (10 mL) was stirred at 80° C. for 0.5 h. The reaction mixture was cooled to RT and NaBH(OAc)₃ (1.20 g, 5.66 mmol) was added. The reaction mixture was stirred at RT for 0.5 h. The reaction mixture was concentrated in vacuo and the residue was purified (PM79) to afford compound 1.425 (0.39 g, 72.8% yield) as a colourless oil.

LCMS (AM3): rt=1.012 min, (456.4 [M+H]⁺), 99% purity.

### 1-(4-Aminobutyl)-N-((2-chloro-[1,1′-biphenyl]-4-yl)methyl)-1H-1,2,3-triazol-4-amine 1.426

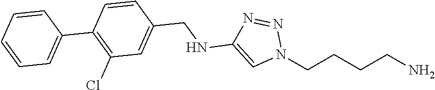 [SMILES: NCCCCn1cc(NCc2ccc(-c3ccccc3)c(Cl)c2)nn1]

A mixture of compound 1.425 (0.39 g, 0.855 mmol) and TFA (5 mL, 67.53 mmol) in DCM (5 mL) was stirred at RT for 0.5 h. The reaction mixture was concentrated in vacuo to afford compound 1.426 (0.5 g, TFA salt) as a brown oil, which was used directly without further purification.

LCMS (AM3): rt=0.819 min, (356.3 [M+H]⁺), 97.2% purity.

### Methyl 5-((4-(4-(((2-chloro-[1,1′-biphenyl]-4-yl)methyl)amino)-1H-1,2,3-triazol-1-yl)butyl)amino)benzo[c][2,6]naphthyridine-8-carboxylate 1.427

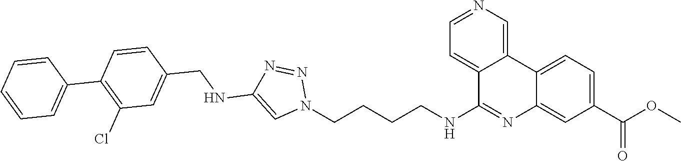 [SMILES: COC(=O)c1ccc2c(c1)nc(NCCCCn1cc(NCc3ccc(-c4ccccc4)c(Cl)c3)nn1)c1ccncc12]

A mixture of compound 1.426 (0.5 g, 1.06 mmol, TFA salt), compound 1.1 (0.23 g, 843 mmol) and DIPEA (3.44 mmol, 0.6 mL) in DMSO (4 mL) was stirred at 80° C. for 14 h. The reaction mixture was filtered and the filtrate was purified (PM40) to afford compound 1.427 (0.25 g, 36.1% yield, 2×TFA salt) as a yellow solid.

LCMS (AM3): rt=0.901 min, (592.4 [M+H]⁺), 100% purity.

### Synthesis of Intermediate 1.431

### tert-Butyl (4-(2-((3-chloro-4-(trifluoromethoxy)benzyl)amino)ethoxy)butyl)carbamate 1.428

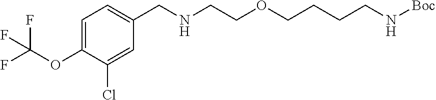 [SMILES: CC(C)(C)OC(=O)NCCCCOCCNCc1ccc(OC(F)(F)F)c(Cl)c1]

To a solution of 3-chloro-4-(trifluoromethoxy)benzaldehyde (CAS 83279-39-4, 1 g, 4.45 mmol) and compound 1.20 (1.28 g, 5.52 mmol) in MeOH (10 mL) was added NaBH(OAc)₃ (5.66 g, 26.72 mmol) at 25° C. The mixture was stirred at 25° C. for 12 h. The mixture was concentrated in vacuo and purified (PM40) to give compound 1.428 (1.2 g, 2.15 mmol, 48.4% yield, TFA salt) as a colourless oil.

LCMS (AM3): rt=0.677 min, (441.0 [M+H]⁺), 99.6% purity

### (9H-Fluoren-9-yl)methyl (2-(4-((tert-butoxycarbonyl)amino)butoxy)ethyl)(3-chloro-4-(trifluoromethoxy)benzyl)carbamate 1.429

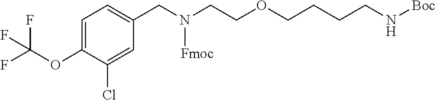

To a solution of compound 1.428 (1.1 g, 1.98 mmol) and NaHCO₃ (832.64 mg, 9.91 mmol) in 1,4-dioxane (4 mL) and H₂O (4 mL) was added Fmoc-Cl (1.03 g, 3.96 mmol) at 0° C. The mixture was then warmed to 25° C. and stirred for 12 h. The mixture was poured into H₂O (70 mL) and the aq. phase was extracted with EA (50 mL×3). The combined organic phase was washed (brine, 70 mL×3), dried (Na₂SO₄), filtered and concentrated in vacuo. The residue was purified (PM6) to give compound 1.429 (720 mg, 1.09 mmol, 54.8% yield) as a light yellow oil.

LCMS (AM3): rt=1.207 min, (663.3 [M+H]⁺), 35.86% purity.

### (9H-Fluoren-9-yl)methyl (2-(4-aminobutoxy)ethyl)(3-chloro-4-(trifluoromethoxy)benzyl)carbamate 1.430

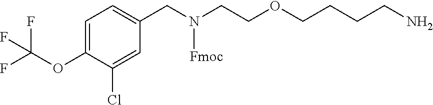

Compound 1.429 (720 mg, 1.09 mmol) in a solution of HCl in 1,4-dioxane (4 M, 8 mL) was stirred at 25° C. for 1 h. The mixture was concentrated in vacuo to afford compound 1.430 (700 mg, HCl salt) as a colourless oil.

LCMS (AM3): rt=0.895 min, (563.3 [M+H]⁺), 83.5% purity.

### Methyl 5-((4-(2-((3-chloro-4-(trifluoromethoxy)benzyl)amino)ethoxy)butyl)amino)benzo[c][2,6]naphthyridine-8-carboxylate 1.431

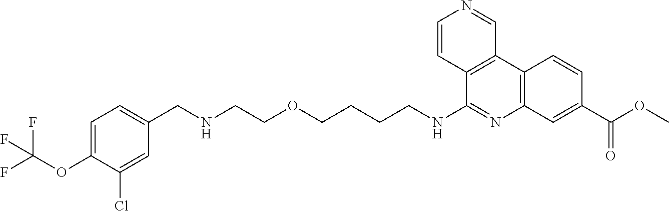 [SMILES: COC(=O)c1ccc2c(c1)nc(NCCCCOCCNCc1ccc(OC(F)(F)F)c(Cl)c1)c1ccncc12]

To a solution of compound 1.1 (320 mg, 1.17 mmol) and compound 1.430 (660.69 mg, 1.17 mmol) in DMSO (10 mL) was added DIPEA (454.99 mg, 3.52 mmol). The mixture was heated to 70° C. and stirred for 12 h. The mixture was poured into H₂O (80 mL) and the aq. phase was extracted with EA (40 mL×3). The combined organic phase was washed (brine, 80 mL×3), dried (Na₂SO₄), filtered and concentrated in vacuo. The residue was purified (PM48) to afford compound 1.431 (347 mg, 565.65 μmol, 48.2% yield, HCl salt) as a yellow solid.

LCMS (AM3): rt=0.625 min, (577.0 [M+H]⁺), 26.6% purity.

### Synthesis of Intermediate 1.436

### 2-(3-(2-Aminoethyl)phenyl)acetonitrile 1.433

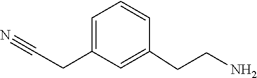 [SMILES: N#CCc1cccc(CCN)c1]

To a solution of 2,2′-(1,3-phenylene)diacetonitrile (1 g, 6.40 mmol) in MeOH (10 mL) was added Raney Ni (548.55 mg, 6.40 mmol) under N₂. The suspension was degassed under vacuum and purged with hydrogen three times. The mixture was then hydrogenated under H₂ atmosphere at 25° C. for 12 h. The reaction mixture was filtered and the filtrate was concentrated to give compound 1.433 (1.39 g) as a yellow oil.

LCMS (AM6): rt=1.087 min, (161.0 [M+H]⁺), 39.9% purity.

### Methyl 5-((3-(cyanomethyl)phenethyl)amino)benzo[c][2,6]naphthyridine-8-carboxylate 1.434

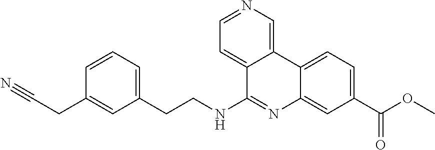 [SMILES: COC(=O)c1ccc2c(c1)nc(NCCc1cccc(CC#N)c1)c1ccncc12]

To a solution of compound 1.1 (750 mg, 2.75 mmol) and compound 1.433 (1.36 g, 8.25 mmol) in DMSO (10 mL) was added DIPEA (1.07 g, 8.25 mmol). The mixture was heated to 70° C. and stirred for 12 h. The residue was poured into H₂O (50 mL) and the aq. phase was extracted with EA (30 mL×3). The combined organic phase was washed (brine, 60 mL×3), dried (Na₂SO₄), filtered and concentrated in vacuo. The residue was purified (PM48) to give compound 1.434 (1.3 g, 2.13 mmol, 77.5% yield) as a yellow oil.

LCMS (AM3): rt=0.827 min, (397.2 [M+H]⁺), 65.0% purity.

### Methyl 5-((3-(2-aminoethyl)phenethyl)amino)benzo[c][2,6]naphthyridine-8-carboxylate 1.435

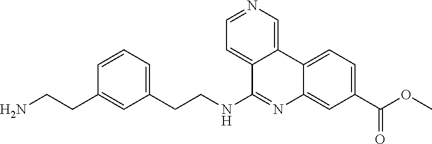 [SMILES: COC(=O)c1ccc2c(c1)nc(NCCc1cccc(CCN)c1)c1ccncc12]

To a solution of compound 1.434 (1.3 g, 3.28 mmol) in MeOH (10 mL) was added Raney Ni (280.93 mg, 3.28 mmol) under N₂. The suspension was degassed and purged with H₂ (×3) The mixture was hydrogenated under H₂ atmosphere at 25° C. for 32 h. The reaction mixture was filtered and the filtrate was concentrated in vacuo. The residue was purified (PM48) to afford compound 1.435 (273 mg, 624.81 μmol, 19.1% yield, HCl salt) as a yellow solid.

LCMS (AM3): rt=0.534 min, (401.0 [M+H]⁺), 52.7% purity.

### Methyl 5-((3-(2-(((2-chloro-[1,1′-biphenyl]-4-yl)methyl)amino)ethyl)phenethyl)amino)benzo[c][2,6]naphthyridine-8-carboxylate 1.436

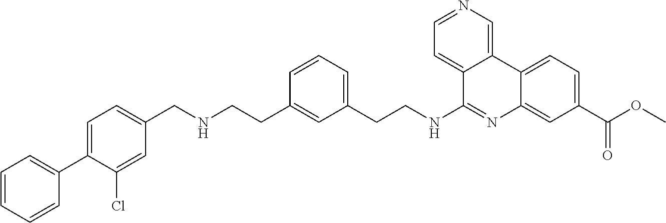 [SMILES: COC(=O)c1ccc2c(c1)nc(NCCc1cccc(CCNCc3ccc(-c4ccccc4)c(Cl)c3)c1)c1ccncc12]

To a solution of compound 1.435 (108 mg, 247.18 μmol) in DCE (2 mL) was added DIPEA (63.89 mg, 494.35 μmol) and 3-chloro-4-phenylbenzaldehyde (CAS 57592-44-6, 56.23 mg, 259.54 μmol), then NaBH₃CN (155.33 mg, 2.47 mmol) was added, and the mixture was stirred at 25° C. for 12 h. The mixture was concentrated in vacuo and purified (PM61) to afford compound 1.436 (140 mg, 200.29 μmol, 81.0% yield) as a yellow solid.

LCMS (AM3): rt=0.762 min, (601.1 [M+H]⁺), 86.1% purity.

### Synthesis of Intermediate 1.442

### tert-Butyl N-[2-[4-(benzyloxycarbonylaminomethyl)-1-piperidyl]ethyl]carbamate 1.438

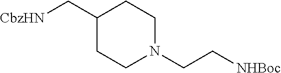 [SMILES: CC(C)(C)OC(=O)NCCN1CCC(CNC(=O)OCc2ccccc2)CC1]

To a solution of benzyl (piperidin-4-ylmethyl)carbamate (3 g, 12.08 mmol) and K₂CO₃ (15.03 g, 108.73 mmol) in DMF (10 mL) was added tert-butyl N-(2-bromoethyl)carbamate (4.06 g, 18.12 mmol). The mixture was stirred at 25° C. for 12 h. The mixture was poured into H₂O (50 mL) and the aq. phase was extracted with EA (50 mL×4). The combined organic phase was washed (brine, 100 mL×3), dried (Na₂SO₄), filtered and concentrated in vacuo. The residue was purified (PM2) to give compound 1.438 (4.3 g, 9.23 mmol, 76.4% yield) as a light yellow solid.

LCMS (AM3): rt=0.771 min, (392.3 [M+H]⁺), 84.4% purity.

### Benzyl ((1-(2-aminoethyl)piperidin-4-yl)methyl)carbamate 1.439

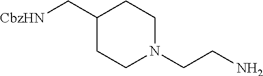 [SMILES: NCCN1CCC(CNC(=O)OCc2ccccc2)CC1]

Compound 1.438 (4.3 g, 10.98 mmol) in a solution of HCl in MeOH (27.5 mL, 4 M) was stirred at 25° C. for 1 h. The mixture was concentrated in vacuo to give compound 1.439 (3.7 g, HCl salt) as a white solid.

LCMS (AM3): rt=0.780 min, (292.2 [M+H]⁺), 45.4% purity.

### Methyl 5-((2-(4-((((benzyloxy)carbonyl)amino)methyl)piperidin-1-yl)ethyl)amino)benzo[c][2,6]naphthyridine-8-carboxylate 1.440

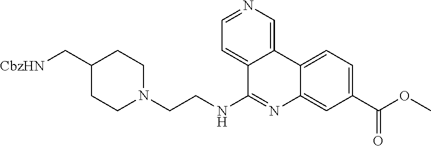 [SMILES: COC(=O)c1ccc2c(c1)nc(NCCN1CCC(CNC(=O)OCc3ccccc3)CC1)c1ccncc12]

To a solution of compound 1.1 (1 g, 3.67 mmol) and compound 1.439 (3.61 g, 11.00 mmol) in DMSO (20 mL) was added DIPEA (1.42 g, 11.00 mmol). The mixture was heated to 80° C. and stirred for 12 h. The mixture was poured into H₂O (100 mL) and the resulting mixture was extracted with EA (50 mL×2). The combined organic phase was washed (brine, 100 mL), dried (Na₂SO₄) and concentrated in vacuo. The obtained residue was purified (PM48) to afford compound 1.440 (1.06 g, 1.90 mmol, 51.8% yield) as a yellow solid.

LCMS (AM3): rt=0.647 min, (528.1 [M+H]⁺), 95.4% purity.

### Methyl 5-((2-(4-(aminomethyl)piperidin-1-yl)ethyl)amino)benzo[c][2,6]naphthyridine-8-carboxylate 1.441

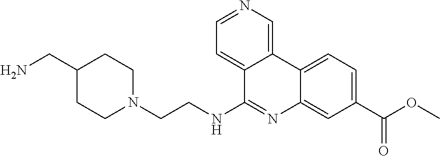 [SMILES: COC(=O)c1ccc2c(c1)nc(NCCN1CCC(CN)CC1)c1ccncc12]

To a solution of compound 1.440 (1.06 g, 2.01 mmol) in EA (20 mL) was added 10% Pd(OH) 2 on carbon (453.30 mg) under N₂. The suspension was degassed under vacuum and purged with hydrogen three times. The mixture was hydrogenated under H₂ atmosphere at 25° C. for 3 h. The catalyst was removed by filtration and the filtrate was concentrated in vacuo. The obtained residue was purified (PM48) to afford compound 1.441 (530 mg, 1.23 mmol, 61.4% yield, HCl salt) as a yellow solid.

LCMS (AM3): rt=0.595 min, (394.1 [M+H]⁺), 75.6% purity.

### Methyl 5-((2-(4-((((2-chloro-[1,1′-biphenyl]-4-yl)methyl)amino)methyl)piperidin-1-yl)ethyl)amino)benzo[c][2,6]naphthyridine-8-carboxylate 1.442

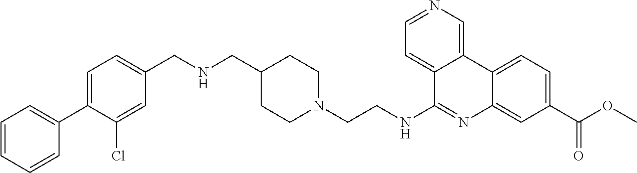 [SMILES: COC(=O)c1ccc2c(c1)nc(NCCN1CCC(CNCc3ccc(-c4ccccc4)c(Cl)c3)CC1)c1ccncc12]

To a solution of compound 1.441 (200 mg, 465.18 μmol) in MeOH (5 mL) was added DIPEA (120.24 mg, 930.36 μmol) and 3-chloro-4-phenylbenzaldehyde (CAS 57592-44-6, 110.87 mg, 511.70 μmol) followed by NaBH₃CN (292.32 mg, 4.65 mmol), the mixture was stirred at 25° C. for 12 h. The mixture was concentrated in vacuo and purified (PM48) to afford compound 1.442 (214 mg, 356.58 μmol, 76.7% yield) as a yellow solid.

LCMS (AM3): rt=0.789 min, (594.3 [M+H]⁺), 99.6% purity.

### Synthesis of Intermediate 1.451

### (E)-tert-Butyl 3-(cyanomethylene)pyrrolidine-1-carboxylate 1.444

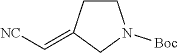 [SMILES: CC(C)(C)OC(=O)N1CC/C(=C\C#N)C1]

To a solution of 2-diethoxyphosphorylacetonitrile (4.78 g, 26.99 mmol) in THF (50 mL) was added potassium tert-butoxide (3.63 g, 32.39 mmol) at 0° C. under N₂. After being stirred for 10 min, tert-butyl 3-oxopyrrolidine-1-carboxylate (CAS 101385-93-7, 5 g, 26.99 mmol) was added and the reaction mixture was warmed to 25° C. and stirred for 12 h. The reaction mixture was diluted with H₂O (100 mL) and the aq. phase was extracted with EA (80 mL×3). The combined organic phase was washed (brine, 100 mL×3), dried (Na₂SO₄), filtered and concentrated in vacuo. The residue was purified (PM8) to give compound 1.444 (2.2 g, 10.46 mmol, 38.7% yield) as a yellow oil.

LCMS (AM3): rt=0.877 min, (153.1 [M−56+H]⁺), 99.5% purity.

### tert-Butyl 3-(2-aminoethyl)pyrrolidine-1-carboxylate 1.445

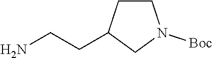 [SMILES: CC(C)(C)OC(=O)N1CCC(CCN)C1]

To a solution of compound 1.444 (3.8 g, 8.95 mmol) in MeOH (50 mL) was added Raney Ni (905.01 mg, 8.95 mmol) and ammonium hydroxide (4.92 mL, 35.80 mmol, 28%) under N₂. The suspension was degassed and purged with H₂ (×3). The mixture was hydrogenated under H₂ atmosphere at 25° C. for 12 h. The catalyst was removed by filtration and the filtrate was concentrated in vacuo to give compound 1.445 (1.8 g) as a yellow oil.

LCMS (AM3): rt=0.696 min, (159.2 [M-tBu+2H]⁺), 52.2% purity.

### tert-Butyl 3-(2-((3-chloro-4-(trifluoromethoxy)benzyl)amino)ethyl)pyrrolidine-1-carboxylate 1.446

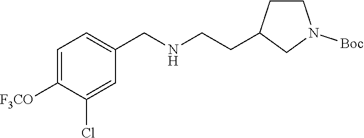 [SMILES: CC(C)(C)OC(=O)N1CCC(CCNCc2ccc(OC(F)(F)F)c(Cl)c2)C1]

To a solution of compound 1.445 (2 g, 9.33 mmol) in MeOH (20 mL) was added 3-chloro-4-(trifluoromethoxy)benzaldehyde (CAS 83279-39-4, 2.10 g, 9.33 mmol). After being stirred at 20° C. for 12 h, NaBH₃CN (3.52 g, 56.00 mmol) was added and stirred at 20° C. for 1 h. The mixture was concentrated in vacuo and purified (PM40) to afford compound 1.446 (1.2 g, 2.84 mmol, 30.4% yield) as a yellow oil.

LCMS (AM3): rt=0.828 min, (367.1 [M-tBu+2H]⁺), 37.7% purity.

### tert-Butyl 3-(2-(N-(3-chloro-4-(trifluoromethoxy)benzyl)-2,2,2-trifluoroacetamido)ethyl)pyrrolidine-1-carboxylate 1.447

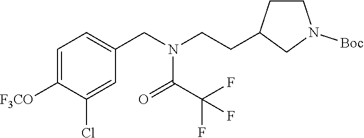 [SMILES: CC(C)(C)OC(=O)N1CCC(CCN(Cc2ccc(OC(F)(F)F)c(Cl)c2)C(=O)C(F)(F)F)C1]

To a solution of compound 1.446 (1.15 g, 2.72 mmol) in DCM (10 mL) was added TFAA (856.78 mg, 4.08 mmol) and TEA (687.96 mg, 6.80 mmol) at 0° C. The mixture was stirred at 0° C. for 1 h. The mixture was concentrated in vacuo and purified (PM40) to afford compound 1.447 (1.5 g) as a brown oil.

LCMS (AM3): rt=1.095 min, (463.1 [M-tBu+2H]⁺), 38.6% purity.

### N-(3-Chloro-4-(trifluoromethoxy)benzyl)-2,2,2-trifluoro-N-(2-(pyrrolidin-3-yl)ethyl)acetamide 1.448

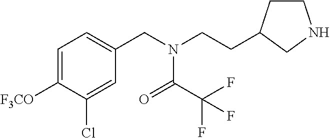 [SMILES: O=C(N(CCC1CCNC1)Cc1ccc(OC(F)(F)F)c(Cl)c1)C(F)(F)F]

To a solution of compound 1.447 (500 mg, 963.62 μmol) in DCM (5 mL) was added TFA (3.08 g, 27.01 mmol) at 20° C. The mixture was stirred at 20° C. for 1 h. The mixture was concentrated in vacuo to afford compound 1.448 (625 mg, TFA salt) as a yellow oil.

LCMS (AM3): rt=0.864 min, (419.1 [M+H]⁺), 93.9% purity.

### tert-Butyl (2-(3-(2-(N-(3-chloro-4-(trifluoromethoxy)benzyl)-2,2,2-trifluoroacetamido)ethyl)pyrrolidin-1-yl)ethyl)carbamate 1.449

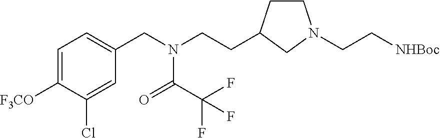 [SMILES: CC(C)(C)OC(=O)NCCN1CCC(CCN(Cc2ccc(OC(F)(F)F)c(Cl)c2)C(=O)C(F)(F)F)C1]

To a solution of compound 1.448 (575 mg, 1.08 mmol) and tert-butyl N-(2-oxoethyl)carbamate (257.69 mg, 1.62 mmol) in DCE (10 mL) was added DIPEA (278.97 mg, 2.16 mmol) and NaBH(OAc)₃ (1.14 g, 5.40 mmol). The mixture was stirred at 20° C. for 2 h. The mixture was concentrated in vacuo and the residue was purified (PM80) to afford compound 1.449 (266 mg, 473.36 μmol, 43.9% yield) as a yellow oil.

LCMS (AM3): rt=0.936 min, (562.2 [M+H]⁺), 19.5% purity.

### N-(2-(1-(2-Aminoethyl) pyrrolidin-3-yl)ethyl)-N-(3-chloro-4-(trifluoromethoxy)benzyl)-2,2,2-trifluoroacetamide 1.450

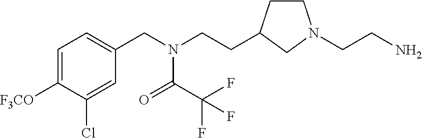 [SMILES: NCCN1CCC(CCN(Cc2ccc(OC(F)(F)F)c(Cl)c2)C(=O)C(F)(F)F)C1]

Compound 1.449 (266 mg, 473.36 μmol) in a solution of HCl in MeOH (4 mL, 4 M) was stirred at 20° C. for 1 h. The mixture was concentrated in vacuo to afford compound 1.450 (120 mg, HCl salt) as a yellow oil.

LCMS (AM3): rt=0.653 min, (461.9 [M+H]⁺), 92.8% purity.

### Methyl 5-((2-(3-(2-(N-(3-chloro-4-(trifluoromethoxy)benzyl)-2,2,2-trifluoroacetamido)ethyl)pyrrolidin-1-yl)ethyl)amino)benzo[c][2,6]naphthyridine-8-carboxylate 1.451

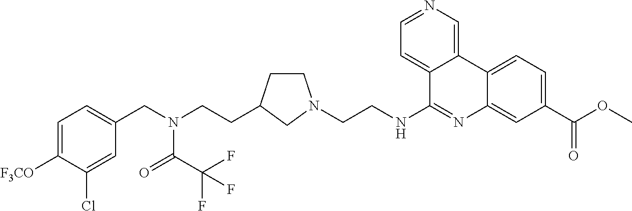 [SMILES: COC(=O)c1ccc2c(c1)nc(NCCN1CCC(CCN(Cc3ccc(OC(F)(F)F)c(Cl)c3)C(=O)C(F)(F)F)C1)c1ccncc12]

To a solution of compound 1.1 (40 mg, 146.69 μmol) and compound 1.450 (94.84 mg, 205.36 μmol) in DMSO (2 mL) was added DIPEA (94.79 mg, 733.44 μmol). The mixture was heated to 80° C. for 12 h. The mixture was diluted with H₂O (50 mL) and extracted with EA (50 mL×2). The combined organic phase was washed (brine, 50 mL), dried over Na₂SO₄ and concentrated in vacuo. The residue was purified (PM40) to afford compound 1.451 (75 mg, 107.44 μmol, 73.2% yield) as a yellow solid.

LCMS (AM3): rt=0.931 min, (698.2 [M+H]⁺),57.5% purity.

### Synthesis of Intermediate 1.458

### tert-Butyl (2-(3-bromopropoxy)ethyl)carbamate 1.453

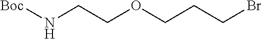 [SMILES: CC(C)(C)OC(=O)NCCOCCCBr]

To a solution of tert-butyl (2-(3-hydroxypropoxy)ethyl)carbamate (ref: US2007/155767A1, 8 g, 36.48 mmol) in DCM (80 mL) was added CBr₄ (18.15 g, 54.73 mmol) and triphenyl phosphine (9.57 g, 36.48 mmol) at 25° C. The resulting mixture was stirred at 25° C. for 12 h. The reaction mixture was poured into H₂O (100 mL) and the aq. phase was extracted with DCM (80 mL×2). The combined organic phase was washed (brine, 100 mL×2), dried (Na₂SO₄), filtered and concentrated in vacuo. The residue was purified (PM7) to afford compound 1.453 (4.3 g, 15.24 mmol, 41.8% yield) as a yellow oil.

¹H NMR (400 MHz, CHCl₃-d) δ: 4.86 (br s, 1H), 3.57 (t, J=5.8 Hz, 2H), 3.53-3.47 (m, 4H), 3.34-3.28 (m, 2H), 2.13-2.07 (quin, 2H), 1.45 (s, 9H) ppm.

### di-tert-Butyl(((disulfanediylbis(propane-3,1-diyl))bis(oxy))bis(ethane-2,1-diyl))dicarbamate 1.454

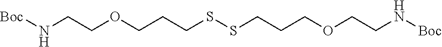 [SMILES: CC(C)(C)OC(=O)NCCOCCCSSCCCOCCNC(=O)OC(C)(C)C]

To a solution of compound 1.453 (1 g, 3.54 mmol) in DMF (10 mL) was added NaSH (993.36 mg, 17.72 mmol) at 0° C. The mixture was warmed to 20° C. and stirred for 1 h. The reaction mixture was poured into H₂O (50 mL) and the aq. phase was extracted with EA (20 mL×4). The combined organic phase was washed (brine, 50 mL×3), dried (Na₂SO₄), filtered and concentrated in vacuo to afford compound 1.454 (1.1 g, 2.35 mmol, 66.2% yield) as a colourless oil.

LCMS (AM3): rt=1.015 min, (469.2 [M+H]⁺), 69.3% purity.

### tert-Butyl (2-(3-(chlorosulfonyl)propoxy)ethyl)carbamate 1.455

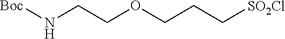 [SMILES: CC(C)(C)OC(=O)NCCOCCCS(=O)(=O)Cl]

To a mixture of compound 1.454 (600 mg, 1.28 mmol) and TBAC (1.42 g, 5.12 mmol) in a solution of H₂O (57.66 μL, 3.20 mmol) and ACN (10 mL) was added NCS (769.28 mg, 5.76 mmol) at 0° C. The mixture was stirred at 0° C. for 1 h. The mixture was concentrated in vacuo to afford compound 1.455 (450 mg) as a colourless oil, which was used without purification.

### tert-Butyl (2-(3-(N-(3-chlorobenzyl)sulfamoyl)propoxy)ethyl)carbamate 1.456

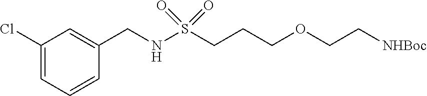 [SMILES: CC(C)(C)OC(=O)NCCOCCCS(=O)(=O)NCc1cccc(Cl)c1]

To a solution of (3-chlorophenyl)methanamine (422.28 mg, 2.98 mmol) and DIPEA (385.42 mg, 2.98 mmol) in THF (5 mL) was added compound 1.455 (450 mg, 1.49 mmol) at 20° C. The mixture was stirred at 20° C. for 1 h. The mixture was concentrated in vacuo and the residue was purified (PM3) to afford compound 1.456 (520 mg, 1.23 mmol, 82.7% yield) as a white solid.

LCMS (AM3): rt=0.915 min, (351.1 [M-tBu+2H]⁺), 96.5% purity.

### 3-(2-Aminoethoxy)-N-(3-chlorobenzyl)propane-1-sulfonamide 1.457

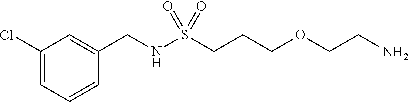 [SMILES: NCCOCCCS(=O)(=O)NCc1cccc(Cl)c1]

Compound 1.456 (520 mg, 1.28 mmol) in a solution of HCl in MeOH (5 mL, 4 M) was stirred at 20° C. for 0.5 h. The mixture was concentrated in vacuo to afford compound 1.457 (420 mg, HCl salt) as a white solid.

LCMS (AM3): rt=0.632 min, (307.1 [M+H]⁺), 95.2% purity.

### Methyl 5-((2-(3-(N-(3-chlorobenzyl)sulfamoyl)propoxy)ethyl)amino)benzo[c][2,6]naphthyridine-8-carboxylate 1.458

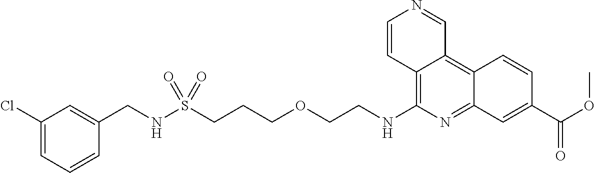 [SMILES: COC(=O)c1ccc2c(c1)nc(NCCOCCCS(=O)(=O)NCc1cccc(Cl)c1)c1ccncc12]

A solution of compound 1.1 (240 mg, 880.13 μmol), compound 1.457 (378.04 mg, 1.23 mmol) and DIPEA (568.74 mg, 4.40 mmol) in DMSO (5 mL) was stirred at 80° C. for 12 h. The mixture was diluted with H₂O (50 mL) and extracted with EA (50 mL×2). The combined organic phase was washed (brine, 50 mL), dried (Na₂SO₄) and concentrated in vacuo. The residue was purified (PM40) to afford compound 1.458 (430 mg, 791.85 μmol, 89.9% yield) as a yellow solid.

LCMS (AM3): rt=0.855 min, (543.2 [M+H]⁺), 39% purity.

### Synthesis of Intermediate 1.464

### tert-Butyl (5-(cyanomethoxy)pentyl)carbamate 1.460

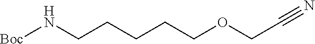 [SMILES: CC(C)(C)OC(=O)NCCCCCOCC#N]

To a solution of tert-butyl (5-hydroxypentyl)carbamate (4 g, 19.68 mmol) in DCM (50 mL) was added 2-bromoacetonitrile (4.72 g, 39.36 mmol), TBAI (1.45 g, 3.94 mmol) and Ag₂O (4.56 g, 19.68 mmol) at 25° C. The reaction mixture was stirred at 25° C. for 12 h. The mixture was filtered and concentrated in vacuo, and the residue was purified (PM8) to afford compound 1.460 (280 mg, 1.16 mmol, 5.9% yield) as a colourless oil.

¹H NMR (400 MHz, CHCl₃-d) δ: 4.56 (br s, 1H), 4.23 (s, 2H), 3.57 (t, J=6.4 Hz, 2H), 3.11 (q, J=6.0 Hz, 2H), 1.67-1.60 (quin, 2H), 1.54-1.42 (m, 2H), 1.44 (s, 9H), 1.42-1.35 (m, 2H) ppm.

### tert-Butyl (5-(2-aminoethoxy)pentyl)carbamate 1.461

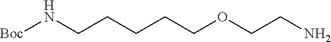 [SMILES: CC(C)(C)OC(=O)NCCCCCOCCN]

To a solution of compound 1.460 (400 mg, 1.65 mmol) in MeOH (10 mL) was added Raney Ni (141.43 mg, 1.65 mmol) and ammonium hydroxide (826.46 mg, 6.60 mmol, 28%) under N₂. The suspension was degassed and purged with H₂ (×3). The mixture was hydrogenated under H₂ at 25° C. for 12 h. The catalyst was removed by filtration and the filtrate was concentrated in vacuo to give compound 1.461 (423 mg) as a light yellow oil, which was used without purification.

### Methyl 5-((2-((5-((tert-butoxycarbonyl)amino)pentyl)oxy)ethyl)amino)benzo[c][2,6]naphthyridine-8-carboxylate 1.462

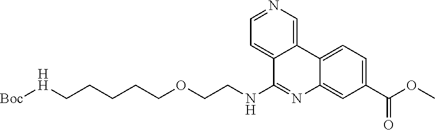

A solution of compound 1.1 (300 mg, 1.10 mmol), compound 1.461 (379.43 mg, 1.54 mmol) and DIPEA (426.56 mg, 3.30 mmol) in DMSO (5 mL) was stirred at 80° C. for 12 h. The mixture was diluted with H₂O (50 mL) and extracted with EA (50 mL×2). The combined organic phase was washed (brine, 50 mL), dried (Na₂SO₄) and concentrated in vacuo. The residue was purified (PM40) to afford compound 1.462 (500 mg, 1.04 mmol, 94.2% yield) as a yellow oil.

LCMS (AM3): rt=0.874 min, (483.2 [M+H]⁺), 72% purity.

### Methyl 5-((2-((5-aminopentyl)oxy)ethyl)amino)benzo[c][2,6]naphthyridine-8-carboxylate 1.463

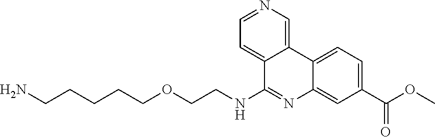 [SMILES: COC(=O)c1ccc2c(c1)nc(NCCOCCCCCN)c1ccncc12]

Compound 1.462 (500 mg, 1.04 mmol) in a solution of HCl in MeOH (5 mL, 4 M) was stirred at 20° C. for 1 h. The mixture was concentrated in vacuo to afford compound 1.463 (450 mg, HCl salt) as a yellow solid.

LCMS (AM3): rt=0.690 min, (383.2 [M+H]⁺),97.9% purity.

### 5-((2-((5-((3-Chlorophenyl)amino)pentyl)oxy)ethyl)amino)benzo[c][2,6]naphthyridine-8-carboxylic acid 1.464

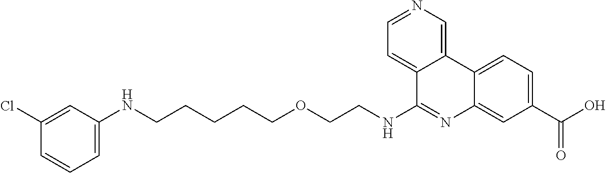 [SMILES: O=C(O)c1ccc2c(c1)nc(NCCOCCCCCNc1cccc(Cl)c1)c1ccncc12]

To a solution of compound 1.463 (400 mg, 954.85 μmol) and 1-chloro-3-iodo-benzene (273.22 mg, 1.15 mmol) in t-BuOH (4 mL) was added sodium tert-butoxide (275.29 mg, 2.86 mmol) and Brettphos Pd G3 (86.56 mg, 95.48 μmol). The reaction mixture was heated to 100° C. and stirred for 12 h under N₂. The mixture was concentrated in vacuo and purified (PM46) to afford compound 1.464 (150 mg, 286.24 μmol, 29.9% yield) as a yellow solid.

LCMS (AM3): rt=0.705 min, (479.0 [M+H]⁺), 91.4% purity.

### Synthesis of Intermediate 1.515

### Tert-butyl (2-(5-nitro-2H-indazol-2-yl)ethyl)carbamate 1.511

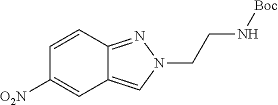 [SMILES: CC(C)(C)OC(=O)NCCn1cc2cc([N+](=O)[O-])ccc2n1]

To a mixture of 5-nitro-2H-indazole (CAS 65750-01-8, 1 g, 6.13 mmol) in ACN (10 mL) was added K₂CO₃ (1.69 g, 12.26 mmol) and tert-butyl (2-bromoethyl)carbamate (2.06 g, 9.19 mmol) at 25° C. The mixture was stirred at 25° C. for 12 h. The mixture was filtered and concentrated in vacuo. The residue was purified (PM2) to afford compound 1.511 (400 mg, 1.31 mmol, 21.3% yield) as a light yellow solid.

LCMS (AM3): rt=0.694 min, (306.9 [M+H]⁺), 99.4% purity.

### 2-(5-Nitro-2H-indazol-2-yl)ethanamine 1.512

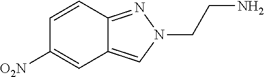 [SMILES: NCCn1cc2cc([N+](=O)[O-])ccc2n1]

A mixture of compound 1.511 (400 mg, 1.31 mmol) in a solution of HCl in 1,4-dioxane (7 mL, 4 M) was stirred at 25° C. for 0.5 h. The precipitate was collected by filtration and dried under vacuum to afford compound 1.512 (300 mg) as a light yellow solid, which was used without purification.

LCMS (AM3): rt=0.233 min, (207.2 [M+H]⁺), 76.1% purity.

### Methyl 5-((2-(5-nitro-2H-indazol-2-yl)ethyl)amino)benzo[c][2,6]naphthyridine-8-carboxylate 1.513

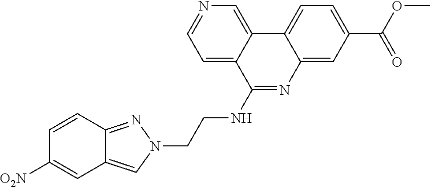 [SMILES: COC(=O)c1ccc2c(c1)nc(NCCn1cc3cc([N+](=O)[O-])ccc3n1)c1ccncc12]

To a mixture of compound 1.512 (300 mg, 1.10 mmol) and compound 1.1 (272.23 mg, 1.32 mmol) in DMSO (5 mL) was added DIPEA (426.56 mg, 3.30 mmol). The mixture was heated to 80° C. and stirred for 12 h. The mixture was poured into H₂O (10 mL) and the aq. phase was extracted with EA (50 mL×3). The combined organic phase was washed (brine, 50 mL), dried (Na₂SO₄), filtered and concentrated in vacuo. The residue was purified (PM2) to afford compound 1.513 (250 mg, 508.56 μmol, 46.2% yield) as a yellow solid.

LCMS (AM3): rt=0.847 min, (443.2 [M+H]⁺), 90.9% purity.

### Methyl 5-((2-(5-amino-2H-indazol-2-yl)ethyl)amino)benzo[c][2,6]naphthyridine-8-carboxylate 1.514

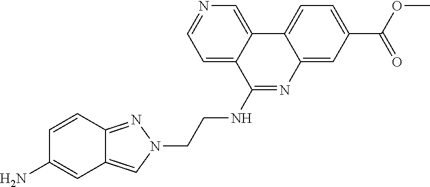 [SMILES: COC(=O)c1ccc2c(c1)nc(NCCn1cc3cc(N)ccc3n1)c1ccncc12]

To a mixture of compound 1.513 (250 mg, 565.07 μmol) and NH₄Cl (302.25 mg, 5.65 mmol) in EtOH (10 mL) and H₂O (2 mL) was added iron powder (63.12 mg, 1.13 mmol) under N₂. The mixture was heated to 65° C. and stirred for 1 h. The mixture was filtered, the filtrate was diluted with H₂O (100 mL) and the aq. phase was extracted with EA (100 mL×2). The combined organic phase was washed (brine, 50 mL), dried (Na₂SO₄), filtered and concentrated in vacuo to afford compound 1.514 (200 mg) as a yellow solid, which was used without further purification.

LCMS (AM3): rt=0.510 min, (412.9 [M+H]⁺), 89.6% purity.

### Methyl 5-((2-(5-(((2-chloro-[1,1′-biphenyl]-4-yl)methyl)amino)-2H-indazol-2-yl)ethyl)amino)benzo[c][2,6]naphthyridine-8-carboxylate 1.515

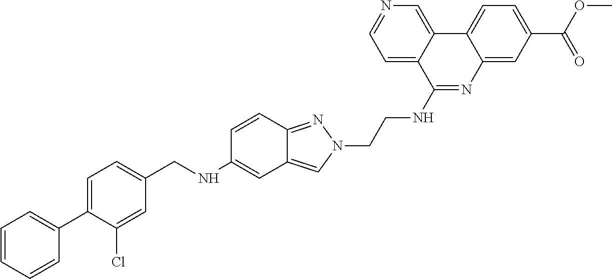 [SMILES: COC(=O)c1ccc2c(c1)nc(NCCn1cc3cc(NCc4ccc(-c5ccccc5)c(Cl)c4)ccc3n1)c1ccncc12]

A mixture of compound 1.514 (190.36 mg, 461.55 μmol) and 3-chloro-4-phenylbenzaldehyde (CAS 57592-44-6, mg, 461.55 μmol) in MeOH (5 mL) was stirred at 25° C. for 1 h before NaBH(OAc)₃ (195.64 mg, 923.09 μmol) was added. The mixture was stirred at 25° C. for 11 h. The mixture was concentrated in vacuo and the residue was purified (PM81) to afford compound 1.515 (70 mg, 70.79 μmol, 15.3% yield) as a yellow solid.

LCMS (AM3): rt=0.940 min, (613.2 [M+H]⁺), 62.3% purity.

### Synthesis of Intermediate 1.540

### Methyl 5-((3-(3-((tert-butoxycarbonyl)amino)propanamido)propyl)amino)benzo[c][2,6]naphthyridine-8-carboxylate 1.538

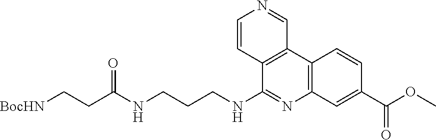 [SMILES: COC(=O)c1ccc2c(c1)nc(NCCCNC(=O)CCNC(=O)OC(C)(C)C)c1ccncc12]

To a solution of 3-((tert-butoxycarbonyl)amino)propanoic acid (609.66 mg, 3.22 mmol) in DMF (15 mL) was added HATU (1.47 g, 3.87 mmol) and DIPEA (832.89 mg, 6.44 mmol). After being stirred at 20° C. for 0.5 h, compound 1.259 (1 g, 3.22 mmol) was added. The reaction mixture was stirred at 20° C. for 2 h. The reaction mixture was diluted with H₂O (50 mL) and extracted with EA (50 mL×2). The combined organic layer was washed (brine, 80 mL×2), dried (Na₂SO₄), filtered and concentrated in vacuo. The residue was purified (PM40) to afford compound 1.538 (940 mg, 1.95 mmol, 60.6% yield) as a yellow oil.

LCMS (AM3): rt=0.793 min, (482.4 [M+H]⁺), 94.2% purity.

### 5-((3-(3-((Tert-butoxycarbonyl)amino)propanamido)propyl)amino)benzo[c][2,6]naphthyridine-8-carboxylic acid 1.539

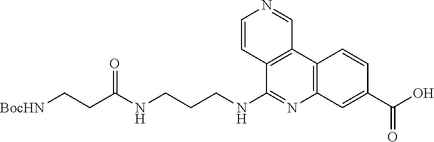 [SMILES: CC(C)(C)OC(=O)NCCC(=O)NCCCNc1nc2cc(C(=O)O)ccc2c2cnccc12]

To a mixture of compound 1.538 (940 mg, 1.58 mmol) in MeOH (8 mL), THF (8 mL) and H₂O (8 mL) was added LiOH·H₂O (132.46 mg, 3.16 mmol) at 20° C. The reaction mixture was stirred at 20° C. for 12 h. The reaction mixture was diluted with H₂O (30 mL) and acidified with aq. HCl (1 M) to pH 4. The precipitate was collected by filtration and dried under vacuum to afford compound 1.539 (700 mg) as a yellow solid, which was used directly without purification.

LCMS (AM3): rt=0.734 min, (468.5 [M+H]⁺), 96.5% purity.

### 5-((3-(3-Aminopropanamido)propyl)amino)benzo[c][2,6]naphthyridine-8-carboxylic acid 1.540

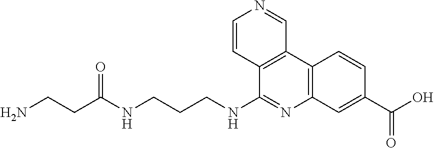 [SMILES: NCCC(=O)NCCCNc1nc2cc(C(=O)O)ccc2c2cnccc12]

A mixture of compound 1.539 (700 mg, 1.50 mmol) in a solution of HCl in 1,4-dioxane (15 mL, 4 M) was stirred at 20° C. for 2 h. The reaction mixture was concentrated in vacuo to afford compound 1.540 (300 mg, 742.83 μmol, 49.6% yield, HCl salt) as a yellow solid, which was used directly without purification.

LCMS (AM5): rt=0.729 min, (368.5 [M+H]⁺), 57.2% purity.

### Synthesis of Intermediate 1.541

### Methyl 5-((3-(3-((3-chloro-4-(trifluoromethoxy)benzyl)amino)propoxy)propyl)amino)benzo[c][2,6]naphthyridine-8-carboxylate 1.541

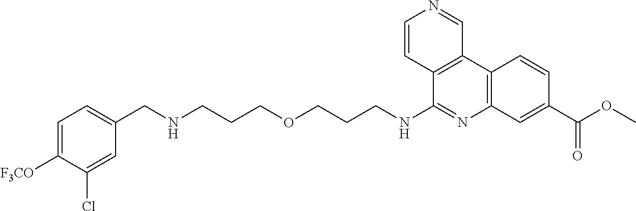 [SMILES: COC(=O)c1ccc2c(c1)nc(NCCCOCCCNCc1ccc(OC(F)(F)F)c(Cl)c1)c1ccncc12]

A mixture of compound 1.113 (500 mg, 1.23 mmol), 3-chloro-4-(trifluoromethoxy)benzaldehyde (CAS 83279-39-4, 277.32 mg, 1.23 mmol) and NaOAc (405.22 mg, 4.94 mmol) in MeOH (15 mL) was stirred at 20° C. for 12 h before NaBH(OAc)₃ (785.18 mg, 3.70 mmol) was added. The reaction mixture was stirred at 20° C. for 3 h. The reaction mixture was poured into H₂O (20 mL) and extracted with EA (30 mL×3). The combined organic phase was washed (brine, 20 mL), dried (Na₂SO₄), filtered and concentrated in vacuo. The residue was purified (PM40) to afford compound 1.541 (400 mg, 578.86 μmol, 46.9% yield, TFA salt) as a yellow oil.

LCMS (AM3): rt=0.815 min, (577.0 [M+H]⁺), 49.0% purity.

### Synthesis of Intermediate 1.544

### Methyl 5-((3-((3-((tert-butoxycarbonyl)amino)propyl)amino)-3-oxopropyl)amino)benzo[c][2,6]naphthyridine-8-carboxylate 1.542

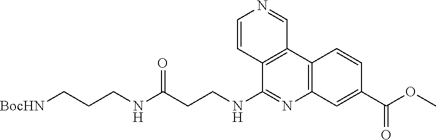 [SMILES: COC(=O)c1ccc2c(c1)nc(NCCC(=O)NCCCNC(=O)OC(C)(C)C)c1ccncc12]

To a solution of Intermediate L (300 mg, 922.17 μmo) in DMF (15 mL) was added DIPEA (178.77 mg, 1.38 mmol) and HATU (525.96 mg, 1.38 mmol). After stirring at 25° C. for 0.5 h, tert-butyl (3-aminopropyl)carbamate (321.36 mg, 1.84 mmol) was added. The reaction mixture was stirred at 25° C. for 1 h. The mixture was poured into H₂O (60 mL) and extracted with EA (30 mL×3). The combined organic phase was washed (brine, 50 mL), dried (Na₂SO₄), filtered and concentrated in vacuo to afford compound 1.542 (342 mg, 710.22 μmol) as a brown oil, which was used directly without further purification.

LCMS (AM3): rt=0.806 min, (482.4 [M+H]⁺), 92.1% purity.

### Methyl 5-((3-((3-aminopropyl)amino)-3-oxopropyl)amino)benzo[c][2,6]naphthyridine-8-carboxylate 1.543

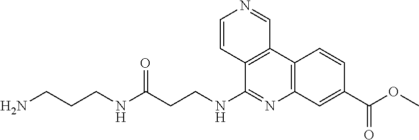 [SMILES: COC(=O)c1ccc2c(c1)nc(NCCC(=O)NCCCN)c1ccncc12]

To a solution of compound 1.542 (342 mg, 710.22 μmol) in DCM (10 mL) was added TFA (15.40 g, 135.06 mmol) at 20° C. The reaction mixture was stirred at 20° C. for 0.1 h. The reaction mixture was concentrated in vacuo to afford compound 1.543 (230 mg, 464.22 μmol) as a yellow solid, which was used directly without purification.

LCMS (AM3): rt=0.692 min, (382.5 [M+H]⁺), 94.6% purity.

### Methyl 5-((3-((3-((3-chloro-4-(trifluoromethoxy)benzyl)amino)propyl)amino)-3-oxopropyl)amino)benzo[c][2,6]naphthyridine-8-carboxylate 1.544

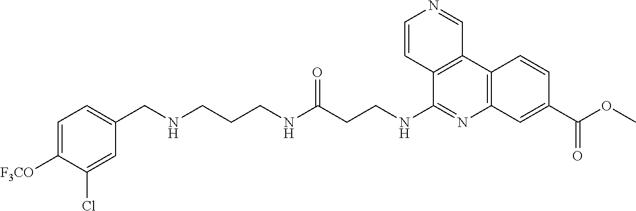 [SMILES: COC(=O)c1ccc2c(c1)nc(NCCC(=O)NCCCNCc1ccc(OC(F)(F)F)c(Cl)c1)c1ccncc12]

A mixture of compound 1.543 (180 mg, 363.31 μmol), 3-chloro-4-(trifluoromethoxy)benzaldehyde (CAS 83279-39-4, 81.59 mg, 363.31 μmol) and DIPEA (93.91 mg, 726.61 μmol) in MeOH (3 mL) was stirred at 20° C. for 12 h before NaBH₃CN (68.49 mg, 1.09 mmol) was added. The reaction mixture was stirred at 20° C. for 3 h. The reaction mixture was filtered and the filtrate was concentrated in vacuo. The residue was purified (PM44) to afford compound 1.544 (200 mg, 284.08 μmol, 78.2% yield, TFA salt) as a yellow solid.

LCMS (AM3): rt=0.815 min, (590.1 [M+H]⁺), 51.9% purity.

### Synthesis of Intermediate 1.549

### Tert-butyl (3-((8-cyanobenzo[c][2,6]naphthyridin-5-yl)amino)propyl)carbamate 1.545

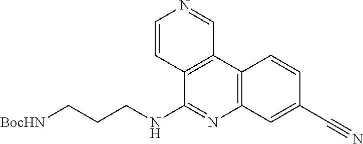 [SMILES: CC(C)(C)OC(=O)NCCCNc1nc2cc(C#N)ccc2c2cnccc12]

To a solution of compound 1.28 (500 mg, 1.26 mmol) in THF (10 mL) was added Burgess reagent (301.31 mg, 1.26 mmol) slowly at 0° C. The reaction mixture was warmed to 20° C. and stirred for 3.5 h. The reaction mixture was filtered and the filtrate was concentrated in vacuo. The residue was purified (PM40) to afford compound 1.545 (380 mg, 773.20 μmol, 61.2% yield, TFA salt) as a yellow solid.

LCMS (AM3): rt=0.836 min, (378.5 [M+H]⁺), 95.2% purity.

### Tert-butyl (3-((8-(2H-tetrazol-5-yl)benzo[c][2,6]naphthyridin-5-yl)amino)propyl)carbamate 1.546

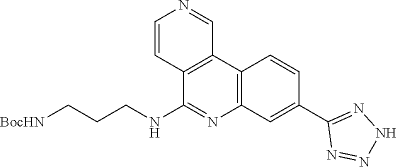 [SMILES: CC(C)(C)OC(=O)NCCCNc1nc2cc(-c3nn[nH]n3)ccc2c2cnccc12]

A mixture of compound 1.545 (270 mg, 715.35 μmol), NH₄Cl (382.65 mg, 7.15 mmol) and sodium azide (930.09 mg, 14.31 mmol) in DMF (20 mL) was stirred at 100° C. for 12 h. The reaction mixture was diluted with H₂O (50 mL) and extracted with EA (40 mL×2). The organic layer was washed (brine, 50 mL×2), dried (Na₂SO₄), filtered and concentrated in vacuo. The residue was purified (PM40) to afford compound 1.546 (150 mg, 356.75 μmol, 49.9% yield) as a yellow solid.

LCMS (AM3): rt=0.798 min, (421.5 [M+H]⁺), 99.5% purity.

### N1-(8-(2H-tetrazol-5-yl)benzo[c][2,6]naphthyridin-5-yl) propane-1,3-diamine 1.547

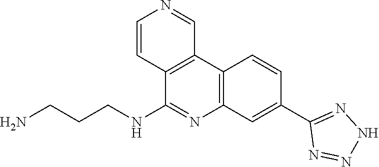 [SMILES: NCCCNc1nc2cc(-c3nn[nH]n3)ccc2c2cnccc12]

A mixture of compound 1.546 (150 mg, 356.75 μmol) in a solution of HCl in MeOH (10 mL, 4 M) was stirred at 20° C. for 0.1 h. The reaction mixture was concentrated in vacuo to afford compound 1.547 (160 mg) as grey solid.

LCMS (AM5): rt=0.730 min, (321.1 [M+H]⁺), 95.0% purity.

### Tert-butyl (3-((3-((8-(2H-tetrazol-5-yl)benzo[c][2,6]naphthyridin-5-yl)amino)propyl)amino)-3-oxopropyl)carbamate 1.548

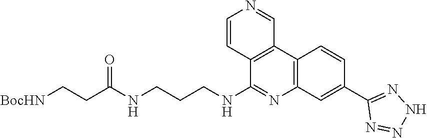 [SMILES: CC(C)(C)OC(=O)NCCC(=O)NCCCNc1nc2cc(-c3nn[nH]n3)ccc2c2cnccc12]

To a solution of 3-((tert-butoxycarbonyl)amino)propanoic acid (84.84 mg, 448.42 μmol) in DMF (10 mL) was added HATU (255.75 mg, 672.62 μmol) and DIPEA (115.91 mg, 896.83 μmol) at 20° C. After being stirred at 20° C. for 0.5 h, compound 1.547 (160 mg, 448.42 μmol, HCl salt) was added. The reaction mixture was stirred at 20° C. for 1.5 h. The reaction mixture was diluted with H₂O (50 mL) and then extracted with EA (50 mL×2). The organic layer was washed (brine, 80 mL), dried (Na₂SO₄), filtered and concentrated in vacuo. The residue was purified (PM40) to afford compound 1.548 (100 mg, 203.44 μmol, 45.4% yield) as a yellow oil.

LCMS (AM3): rt=0.783 min, (492.4 [M+H]⁺), 71.4% purity.

### N-(3-((8-(2H-tetrazol-5-yl)benzo[c][2,6]naphthyridin-5-yl)amino)propyl)-3-aminopropanamide 1.549

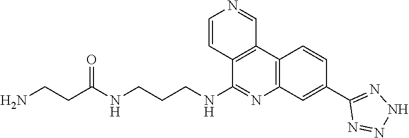 [SMILES: NCCC(=O)NCCCNc1nc2cc(-c3nn[nH]n3)ccc2c2cnccc12]

A mixture of compound 1.548 (100 mg, 203.44 μmol) in a solution of HCl in MeOH (2 mL, 4 M) was stirred at 20° C. for 0.1 h. The reaction mixture was concentrated in vacuo to afford compound 1.549 (100 mg, HCl salt) as a yellow oil.

LCMS (AM5): rt=0.756 min, (392.4 [M+H]⁺), 94.4% purity.

### Synthesis of Intermediate 1.551

### 5-((3-Aminopropyl)amino)benzo[c][2,6]naphthyridine-8-carbonitrile 1.550

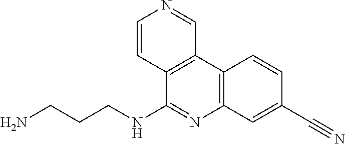 [SMILES: N#Cc1ccc2c(c1)nc(NCCCN)c1ccncc12]

To a solution of compound 1.545 (300 mg, 794.83 μmol) in DCM (15 mL) was added TFA (23.10 g, 202.59 mmol) at 20° C. The reaction mixture was stirred at 20° C. for 0.1 h. The reaction mixture was concentrated in vacuo to afford compound 1.550 (450 mg, TFA salt) as a yellow oil.

LCMS (AM5): rt=0.773 min, (278.6 [M+H]⁺), 26.9% purity.

### Tert-butyl 3-chloro-4-(trifluoromethoxy)benzyl(3-((3-((8-cyanobenzo[c][2,6]naphthyridin-5-yl)amino)propyl)amino)-3-oxopropyl)carbamate 1.551

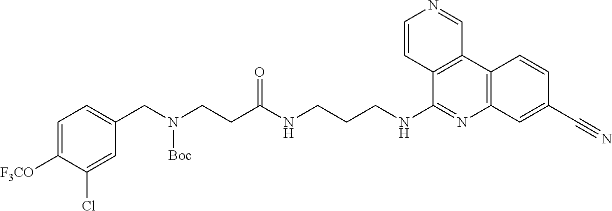 [SMILES: CC(C)(C)OC(=O)N(CCC(=O)NCCCNc1nc2cc(C#N)ccc2c2cnccc12)Cc1ccc(OC(F)(F)F)c(Cl)c1]

To a solution of compound 1.243 (548.87 mg, 1.38 mmol) in DMF (20 mL) was added EDCI (330.65 mg, 1.72 mmol), HOBt (233.06 mg, 1.72 mmol) and DIPEA (222.92 mg, 1.72 mmol) at 20° C. After being stirred for 0.5 h, compound 1.550 (450 mg, 1.15 mmol, TFA salt) was added. The reaction mixture was stirred at 20° C. for 3 h. The reaction mixture was diluted with H₂O (40 mL) and then extracted with EA (40 mL×2). The organic layer was washed (brine, 80 mL×2), dried (Na₂SO₄), filtered and concentrated in vacuo to give compound 1.551 (800 mg) as a yellow oil, which was used directly without purification.

LCMS (AM3): rt=0.975 min, (657.1 [M+H]⁺), 91.8% purity.

### Synthesis of Intermediate 1.552

### 5-((2-(4-(2-((Tert-butoxycarbonyl)((2-chloro-[1,1′-biphenyl]-4-yl)methyl)amino)ethyl)-1H-1,2,3-triazol-1-yl)ethyl)amino)benzo[c][2,6]naphthyridine-8-carboxylic acid 1.552

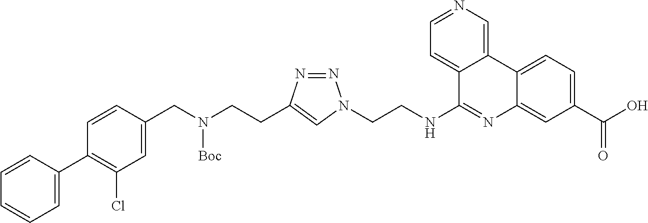 [SMILES: CC(C)(C)OC(=O)N(CCc1cn(CCNc2nc3cc(C(=O)O)ccc3c3cnccc23)nn1)Cc1ccc(-c2ccccc2)c(Cl)c1]

To a mixture of compound 1.171 (500 mg, 496.14 μmol) in THF (10 mL), MeOH (10 mL) and H₂O (10 mL) was added LiOH·H₂O (41.64 mg, 992.28 μmol) at 20° C. The reaction mixture was stirred at 20° C. for 12 h. The organic solvent was removed under reduced pressure and the aq. phase was acidified with aq. 1 N HCl to pH 5. The mixture was then extracted with EA (50 mL×2). The combined organic phase was washed (brine, 50 mL), dried (Na₂SO₄) and concentrated to afford compound 1.552 (300 mg) as a yellow solid.

LCMS (AM3): rt=0.961 min, (678.2 [M+H]⁺), 81.7% purity.

### Synthesis of Intermediate 1.554

### N-((2-Chloro-[1,1′-biphenyl]-4-yl)methyl)prop-2-yn-1-amine 1.553

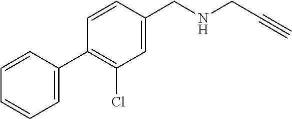 [SMILES: C#CCNCc1ccc(-c2ccccc2)c(Cl)c1]

A solution of 3-chloro-4-phenylbenzaldehyde (CAS 57592-44-6, 1 g, 4.62 mmol) and prop-2-yn-1-amine (254.21 mg, 4.62 mmol) in MeOH (15 mL) was stirred at 20° C. for 12 h, then NaBH(OAc)₃ (978.21 mg, 4.62 mmol) was added. The reaction mixture was stirred at 20° C. for 3 h. The reaction mixture was concentrated in vacuo to afford compound 1.553 (1 g,) as a yellow oil, which was used directly without purification.

LCMS (AM3): rt=0.815 min, (256.6 [M+H]⁺), 55.4% purity.

### Tert-butyl ((2-chloro-[1,1′-biphenyl]-4-yl)methyl)(prop-2-yn-1-yl)carbamate 1.554

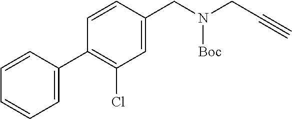 [SMILES: C#CCN(Cc1ccc(-c2ccccc2)c(Cl)c1)C(=O)OC(C)(C)C]

To a solution of compound 1.553 (1 g, 3.91 mmol) in MeOH (15 mL) was added NaHCO₃ (328.48 mg, 3.91 mmol) and Boc₂O (853.39 mg, 3.91 mmol) at 20° C. The reaction mixture was stirred at 20° C. for 1 h. The reaction mixture was concentrated in vacuo and purified (PM8) to afford compound 1.554 (1 g, 2.81 mmol, 71.9% yield) as a yellow oil.

LCMS (AM3): rt=1.126 min, (300.4 [M+H−55]⁺), 86.3% purity.

### Synthesis of Intermediate 1.557

### Methyl 5-((3-azidopropyl)amino)benzo[c][2,6]naphthyridine-8-carboxylate 1.555

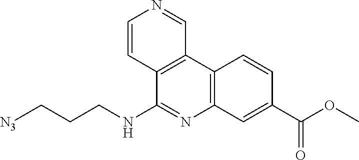 [SMILES: COC(=O)c1ccc2c(c1)nc(NCCCN=[N+]=[N-])c1ccncc12]

A mixture of compound 1.1 (0.5 g, 1.83 mmol), 3-azidopropan-1-amine hydrochloride (183.58 mg, 1.83 mmol) (Chemistry; A European Journal, 2017, 23, (27), 6535-6543) and DIPEA (473.96 mg, 3.67 mmol) in DMSO (20 mL) was stirred at 70° C. for 12 h. The mixture was poured into H₂O (100 mL), and the precipitate was collected by filtration and dried under vacuum to afford compound 1.555 (380 mg, 1.13 mmol, 61.6% yield) as a brown solid.

LCMS (AM3): rt=0.786 min, (337.0 [M+H]⁺), 79.0% purity.

### Methyl 5-((3-(4-(((tert-butoxycarbonyl) ((2-chloro-[1,1′-biphenyl]-4-yl)methyl)amino)methyl)-1H-1,2,3-triazol-1-yl)propyl)amino)benzo[c][2,6]naphthyridine-8-carboxylate 1.556

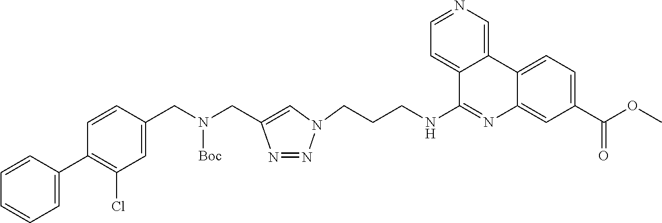 [SMILES: COC(=O)c1ccc2c(c1)nc(NCCCn1cc(CN(Cc3ccc(-c4ccccc4)c(Cl)c3)C(=O)OC(C)(C)C)nn1)c1ccncc12]

A mixture of compound 1.554 (402.04 mg, 1.13 mmol), compound 1.555 (380 mg, 1.13 mmol) and CuI (43.03 mg, 225.96 μmol) in a mixture solvent of DMF (21 mL) and MeOH (3 mL) was stirred at 100° C. for 12 h. The reaction mixture was diluted with H₂O (50 mL) and then extracted with EA (60 mL×2). The organic layer was washed (brine, 80 mL), dried (Na₂SO₄), filtered and concentrated in vacuo. The residue was purified (PM40) to afford compound 1.556 (300 mg, 433.40 μmol, 38.4% yield) as a yellow solid.

LCMS (AM3): rt=0.982 min, (692.2 [M+H]⁺), 56.1% purity.

### Tert-butyl ((1-(3-((8-carbamoylbenzo[c][2,6]naphthyridin-5-yl)amino)propyl)-1H−1,2,3-triazol-4-yl)methyl)((2-chloro-[1,1′-biphenyl]-4-yl)methyl)carbamate 1.557

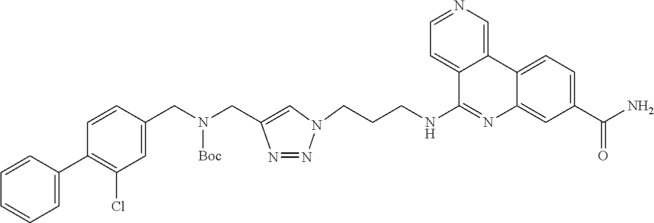 [SMILES: CC(C)(C)OC(=O)N(Cc1ccc(-c2ccccc2)c(Cl)c1)Cc1cn(CCCNc2nc3cc(C(N)=O)ccc3c3cnccc23)nn1]

A solution of compound 1.556 (300 mg, 433.40 μmol) in a solution of NH₃ in MeOH (10 mL, 7 M) was stirred in a sealed tube at 90° C. for 12 h. The reaction mixture was concentrated in vacuo to afford compound 1.557 (200 mg, 295.34 μmol, 68.1% yield) as a yellow solid.

LCMS (AM3): rt=0.906 min, (677.3 [M+H]⁺), 72.1% purity

### Synthesis of Intermediate 1.562

### Methyl 5-((2-((4-(((benzyloxy)carbonyl)amino)butyl)amino)ethyl)amino)benzo[c][2,6]naphthyridine-8-carboxylate 1.558

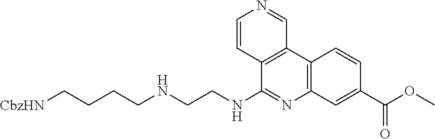 [SMILES: COC(=O)c1ccc2c(c1)nc(NCCNCCCCNC(=O)OCc1ccccc1)c1ccncc12]

A solution of Intermediate A (1 g, 3.00 mmol), TEA (608.14 mg, 6.01 mmol) and benzyl (4-oxobutyl)carbamate (864.31 mg, 3.91 mmol) in MeOH (20 mL) was stirred at 20° C. for 12.5 h, then NaBH(OAc)₃ (1.91 g, 9.01 mmol) was added. The reaction mixture was stirred at 20° C. for 3 h. The reaction mixture was concentrated in vacuo and purified (PM40) to afford compound 1.558 (1.5 g, 2.44 mmol, 81.1% yield, TFA salt) as a yellow oil.

LCMS (AM3): rt=0.788 min, (502.2 [M+H]⁺), 45.7% purity.

### Methyl 5-((2-((4-(((benzyloxy)carbonyl)amino)butyl)(tert-butoxycarbonyl)amino)ethyl)amino)benzo[c][2,6]naphthyridine-8-carboxylate 1.559

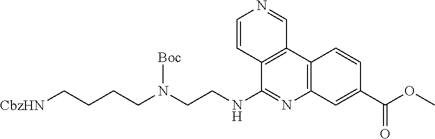 [SMILES: COC(=O)c1ccc2c(c1)nc(NCCN(CCCCNC(=O)OCc1ccccc1)C(=O)OC(C)(C)C)c1ccncc12]

A solution of compound 1.558 (1.3 g, 2.59 mmol), Boc₂O (565.66 mg, 2.59 mmol) and NaHCO₃ (435.46 mg, 5.18 mmol) in MeOH (20 mL) was stirred at 20° C. for 5 h. The reaction mixture was filtered and concentrated in vacuo. The residue was purified (PM40) to afford compound 1.559 (0.31 g, 433.13 μmol, 16.7% yield, TFA salt) as a yellow oil.

LCMS (AM3): rt=0.922 min, (602.3 [M+H]⁺), 41.3% purity.

### 5-((2-((4-(((Benzyloxy)carbonyl)amino)butyl)(tert-butoxycarbonyl)amino)ethyl)amino)benzo[c][2,6]naphthyridine-8-carboxylic acid 1.560

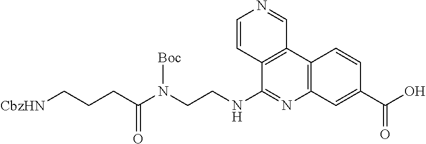 [SMILES: CC(C)(C)OC(=O)N(CCNc1nc2cc(C(=O)O)ccc2c2cnccc12)C(=O)CCCNC(=O)OCc1ccccc1]

To a mixture of compound 1.559 (310 mg, 515.21 μmol) in THF (10 mL), MeOH (10 mL) and H₂O (10 mL) was added LiOH·H₂O (43.24 mg, 1.03 mmol) at 20° C. The reaction mixture was stirred at 20° C. for 2 h. The organic solvents were removed under reduced pressure and then the aq. phase was acidified with aq. 1 N HCl to pH 6. The mixture was extracted with EA (80 mL×2) and the combined organic phase was washed (brine, 80 mL), dried (Na₂SO₄), filtered and concentrated in vacuo to afford compound 1.560 (300 mg, 510.50 μmol, 99.1% yield) as a yellow oil, which was used directly without purification.

LCMS (AM3): rt=0.858 min, (588.2 [M+H]⁺), 90.1% purity.

### 5-((2-((4-Aminobutyl)(tert-butoxycarbonyl)amino)ethyl)amino)benzo[c][2,6]naphthyridine-8-carboxylic acid 1.561

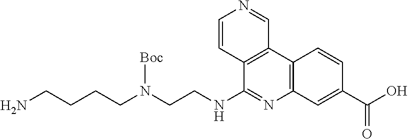 [SMILES: CC(C)(C)OC(=O)N(CCCCN)CCNc1nc2cc(C(=O)O)ccc2c2cnccc12]

To a solution of compound 1.560 (300 mg, 510.50 μmol) in MeOH (10 mL) was added 10% palladium on carbon (50 mg) at 20° C. The resulting suspension was hydrogenated under one atmosphere H₂ at 20° C. for 1 h. The catalyst was removed by filtration and the filtrate was concentrated to afford compound 1.561 (200 mg, 440.98 μmol, 86.4% yield) as a yellow oil.

LCMS (AM3): rt=0.742 min, (454.2 [M+H]⁺), 90.0% purity.

### 5-((2-((Tert-butoxycarbonyl)(4-(((5-(trifluoromethyl)-1H-indol-2-yl)methyl)amino)butyl)amino)ethyl)amino)benzo[c][2,6]naphthyridine-8-carboxylic acid 1.562

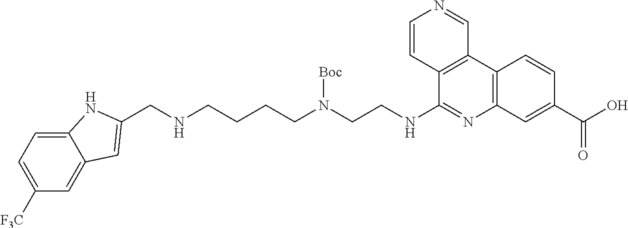 [SMILES: CC(C)(C)OC(=O)N(CCCCNCc1cc2cc(C(F)(F)F)ccc2[nH]1)CCNc1nc2cc(C(=O)O)ccc2c2cnccc12]

A solution of compound 1.561 (150 mg, 330.74 μmol), DIPEA (85.49 mg, 661.47 μmol) and 5-(trifluoromethyl)-1H-indole-2-carbaldehyde (CAS 1367793-79-0, 70.50 mg, 330.74 μmol) in MeOH (3 mL) was stirred at 20° C. for 12.5 h, then NaBH₃CN (62.35 mg, 992.21 μmol) was added. The reaction mixture was stirred at 20° C. for 3 h. The reaction mixture was filtered and concentrated in vacuo. The residue was purified (PM82) to afford compound 1.562 (30 mg, 46.10 μmol, 13.9% yield) as a white solid.

LCMS (AM3): rt=0.861 min, (651.3 [M+H]⁺), 55.2% purity.

### Synthesis of Intermediate 1.569

### Methyl 5-((2-(4-((tert-butoxycarbonyl)amino) butanamido)ethyl)amino)benzo[c][2,6]naphthyridine-8-carboxylate 1.567

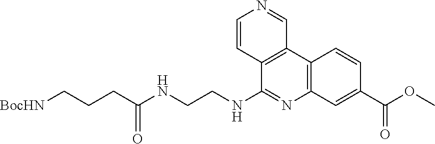 [SMILES: COC(=O)c1ccc2c(c1)nc(NCCNC(=O)CCCNC(=O)OC(C)(C)C)c1ccncc12]

To a solution of 4-((tert-butoxycarbonyl)amino)butanoic acid (250 mg, 1.23 mmol) in DMF (10 mL) was added HATU (701.58 mg, 1.85 mmol) and DIPEA (317.96 mg, 2.46 mmol) at 20° C. After being stirred for 0.1 h, Intermediate A (491.23 mg, 1.48 mmol, HCl salt) was added. The mixture was stirred at 20° C. for additional 2 h. The reaction mixture was diluted with H₂O (100 mL) and then extracted with EA (50 mL×2). The combined organic phase was washed (brine, 100 mL), dried (Na₂SO₄) and concentrated to afford compound 1.567 (550 mg, 923.49 μmol, 75.1% yield) as a yellow solid, which was used directly without purification.

LCMS (AM3): rt=0.821 min, (482.2 [M+H]⁺), 98.0% purity.

### Methyl 5-((2-(4-aminobutanamido)ethyl)amino)benzo[c][2,6]naphthyridine-8-carboxylate 1.568

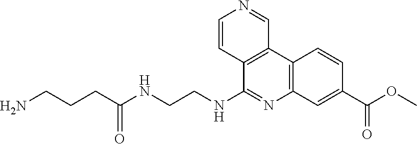 [SMILES: COC(=O)c1ccc2c(c1)nc(NCCNC(=O)CCCN)c1ccncc12]

To a solution of compound 1.567 (550 mg, 1.14 mmol) in MeOH (15 mL) was added a solution of HCl in MeOH (15 mL, 4 M) at 20° C. The reaction mixture was stirred at 20° C. for 0.1 h. The reaction mixture was concentrated in vacuo and purified (PM40) to afford compound 1.568 (380 mg, 996.26 μmol, 87.2% yield) as a yellow oil.

LCMS (AM3): rt=0.788 min, (382.2 [M+H]⁺), 62.0% purity.

### Methyl 5-((2-(4-((3-chloro-4-(trifluoromethoxy)benzyl)amino)butanamido)ethyl)amino)benzo[c][2,6]naphthyridine-8-carboxylate 1.569

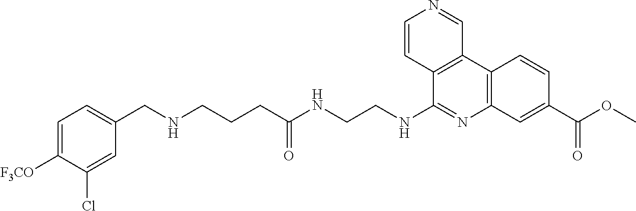 [SMILES: COC(=O)c1ccc2c(c1)nc(NCCNC(=O)CCCNCc1ccc(OC(F)(F)F)c(Cl)c1)c1ccncc12]

A mixture of compound 1.568 (360 mg, 861.47 μmol), DIPEA (222.68 mg, 1.72 mmol) and 3-chloro-4-(trifluoromethoxy)benzaldehyde (193.46 mg, 861.47 μmol) in MeOH (10 mL) was stirred at 20° C. for 1.1 h, then NaBH₃CN (162.41 mg, 2.58 mmol) was added. The reaction mixture was stirred at 20° C. for 0.5 h. The reaction mixture was filtered and the filtrate was concentrated in vacuo. The residue was purified (PM83) to afford compound 1.569 (300 mg, 508.48 μmol, 59.0% yield) as a grey solid.

LCMS (AM3): rt=0.817 min, (590.2 [M+H]⁺), 65.5% purity.

### Synthesis of Intermediate 1.570

### 5-((2-((Tert-butoxycarbonyl)(4-((3-chloro-4-(trifluoromethoxy)benzyl)amino)butyl)amino)ethyl)amino)benzo[c][2,6]naphthyridine-8-carboxylic acid 1.570

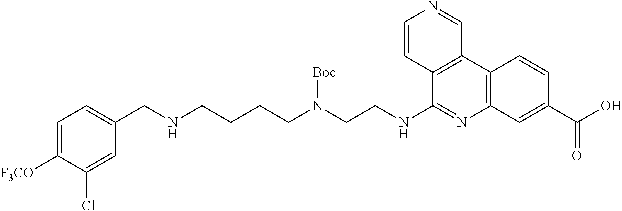 [SMILES: CC(C)(C)OC(=O)N(CCCCNCc1ccc(OC(F)(F)F)c(Cl)c1)CCNc1nc2cc(C(=O)O)ccc2c2cnccc12]

A mixture of compound 1.561 (95 mg, 167.38 μmol), DIPEA (21.63 mg, 167.38 μmol) and 3-chloro-4-(trifluoromethoxy)benzaldehyde (CAS 83279-39-4, 37.59 mg, 167.38 μmol) in MeOH (3 mL) was stirred at 20° C. for 12.5 h, then NaBH₃CN (10.52 mg, 167.38 μmol) was added. The reaction mixture was stirred at 20° C. for 3 h. The reaction mixture was filtered and the filtrate was concentrated in vacuo. The residue was purified (PM84) to afford compound 1.570 (50 mg, 64.42 μmol, 38.5% yield, TFA salt) as a yellow solid.

LCMS (AM3): rt=0.840 min, (662.2 [M+H]⁺), 27.8% purity.

### Synthesis of Intermediate 1.572

### N-((2-chloro-[1,1′-biphenyl]-4-yl)methyl)-N-(2-(1-(2-(N-(8-cyanobenzo[c][2,6]naphthyridin-5-yl)-2,2,2-trifluoroacetamido)ethyl)-1H-1,2,3-triazol-4-yl)ethyl)-2,2,2-trifluoroacetamide 1.571

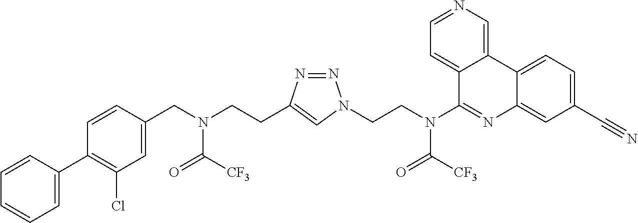 [SMILES: N#Cc1ccc2c(c1)nc(N(CCn1cc(CCN(Cc3ccc(-c4ccccc4)c(Cl)c3)C(=O)C(F)(F)F)nn1)C(=O)C(F)(F)F)c1ccncc12]

To a solution of EXAMPLE 51 (300 mg, 519.86 μmol) in DCM (20 mL) was added TFAA (218.37 mg, 1.04 mmol) and TEA (263.02 mg, 2.60 mmol) at 20° C. The reaction mixture was stirred at 20° C. for 0.1 h. The reaction mixture was concentrated in vacuo and purified (PM85) to afford compound 1.571 (60 mg, 69.36 μmol, 13.3% yield) as a yellow oil.

LCMS (AM3): rt=1.074 min, (751.2 [M+H]⁺), 51.8% purity.

### N-(2-(1-(2-((8-(2H-tetrazol-5-yl)benzo[c][2,6]naphthyridin-5-yl)amino)ethyl)-1H-1,2,3-triazol-4-yl)ethyl)-N-((2-chloro-[1,1′-biphenyl]-4-yl)methyl)-2,2,2-trifluoroacetamide 1.572

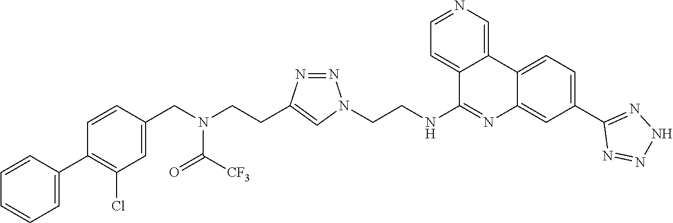 [SMILES: O=C(N(CCc1cn(CCNc2nc3cc(-c4nn[nH]n4)ccc3c3cnccc23)nn1)Cc1ccc(-c2ccccc2)c(Cl)c1)C(F)(F)F]

A mixture of compound 1.571 (60 mg, 79.89 μmol), sodium azide (50 mg, 769.11 μmol) and NH₄Cl (42.73 mg, 798.85 μmol) in DMF (10 mL) was stirred at 100° C. for 12 h. The reaction mixture was diluted with H₂O (50 mL) and extracted with EA (50 mL×2). The combined organic phase was washed (brine, 100 mL), dried (Na₂SO₄), filtered and concentrated. The residue was purified (PM40) to afford compound 1.572 (45 mg, 55.41 μmol, 69.4% yield, TFA salt) as a yellow solid.

LCMS (AM3): rt=0.939 min, (698.4 [M+H]⁺), 84.4% purity.

### Synthesis of Intermediate 1.579

### (R)-Benzyl 3-(3-((tert-butoxycarbonyl)amino)propoxy)pyrrolidine-1-carboxylate 1.575

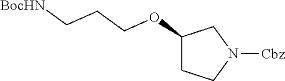 [SMILES: CC(C)(C)OC(=O)NCCCO[C@@H]1CCN(C(=O)OCc2ccccc2)C1]

To a solution of (R)-benzyl 3-hydroxypyrrolidine-1-carboxylate (1 g, 4.52 mmol) in DMF (15 mL) was added NaH (198.85 mg, 4.97 mmol, 60% dispersion in oil) slowly at 0° C. After being stirred for 0.5 h, tert-butyl (3-bromopropyl)carbamate (1.18 g, 4.97 mmol) was added. The reaction mixture was stirred at 0° C. for 3 h. The mixture was diluted with iced H₂O (50 mL) slowly and then extracted with EA (80 mL×2). The combined organic phase was washed (brine, 100 mL), dried (Na₂SO₄), filtered and concentrated in vacuo. The residue was purified (PM2) to afford compound 1.575 (820 mg, 2.17 mmol, 47.9% yield) as a colourless oil.

¹H NMR (400 MHz, CHCl₃-d) δ: 7.40-7.28 (m, 5H), 5.20-5.09 (m, 2H), 4.77 (br s, 1H), 4.05-3.98 (m, 1H), 3.54-3.42 (m, 6H), 3.24-3.12 (m, 2H), 2.05-1.86 (m, 2H), 1.77-1.70 (quin, 2H), 1.43 (s, 9H) ppm.

### (R)-Tert-butyl (3-(pyrrolidin-3-yloxy)propyl)carbamate 1.576

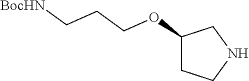 [SMILES: CC(C)(C)OC(=O)NCCCO[C@@H]1CCNC1]

To a solution of compound 1.575 (700 mg, 1.85 mmol) in MeOH (8 mL) was added 10% Pd/C (100 mg) at 20° C. The resulting suspension was hydrogenated under one atmosphere H₂ pressure at 20° C. for 12 h. The catalyst was removed by filtration and the filtrate was concentrated to afford compound 1.576 (460 mg) as a colourless oil, which was used directly without purification.

### (R)-methyl 5-(3-(3-((tert-butoxycarbonyl)amino)propoxy)pyrrolidin-1-yl)benzo[c][2,6]naphthyridine-8-carboxylate 1.577

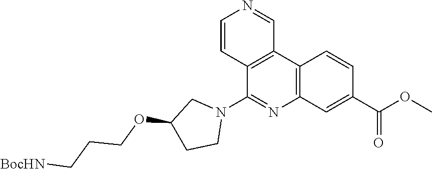 [SMILES: COC(=O)c1ccc2c(c1)nc(N1CC[C@@H](OCCCNC(=O)OC(C)(C)C)C1)c1ccncc12]

A mixture of compound 1.576 (460 mg, 1.88 mmol), compound 1.1 (513.39 mg, 1.88 mmol) and DIPEA (486.65 mg, 3.77 mmol) in DMSO (10 mL) was stirred at 80° C. for 12 h. The reaction mixture was diluted with H₂O (50 mL) and extracted with EA (50 mL×2). The combined organic layer was washed (brine, 80 mL), dried (Na₂SO₄), filtered and concentrated in vacuo. The residue was purified (PM48) to afford compound 1.577 (600 mg, 1.25 mmol, 66.3% yield) as a dark brown oil.

LCMS (AM3): rt=0.844 min, (481.1 [M+H]⁺), 80.2% purity.

### (R)-Methyl 5-(3-(3-aminopropoxy)pyrrolidin-1-yl)benzo[c][2,6]naphthyridine-8-carboxylate 1.578

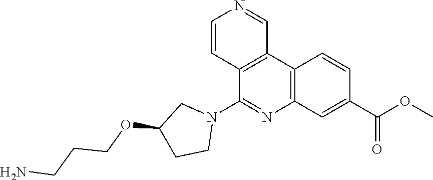 [SMILES: COC(=O)c1ccc2c(c1)nc(N1CC[C@@H](OCCCN)C1)c1ccncc12]

To a solution of compound 1.577 (600 mg, 1.25 mmol) in MeOH (5 mL) was added a solution of HCl in MeOH (10 mL, 4 M) at 20° C. The reaction mixture was stirred at 20° C. for 1 h. The reaction mixture was concentrated in vacuo to afford compound 1.578 (600 mg, HCl salt) as a brown solid, which was used directly without purification.

LCMS (AM5): rt=0.806 min, (381.3 [M+H]⁺), 74.8% purity.

### (R)-Methyl 5-(3-(3-((3-chloro-4-(trifluoromethoxy)benzyl)amino)propoxy)pyrrolidin-1-yl)benzo[c][2,6]naphthyridine-8-carboxylate 1.579

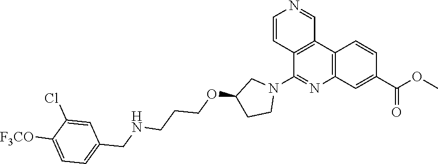 [SMILES: COC(=O)c1ccc2c(c1)nc(N1CC[C@@H](OCCCNCc3ccc(OC(F)(F)F)c(Cl)c3)C1)c1ccncc12]

A mixture of compound 1.578 (600 mg, 1.22 mmol), DIPEA (474.93 mg, 3.67 mmol) and 3-chloro-4-(trifluoromethoxy)benzaldehyde (CAS 83279-39-4, 288.83 mg, 1.29 mmol) in MeOH (15 mL) was stirred at 20° C. for 12 h, then NaBH(OAc)₃ (778.84 mg, 3.67 mmol) was added. The reaction mixture was stirred at 20° C. for 3 h. The reaction mixture was filtered and the filtrate was concentrated in vacuo. The residue was purified (PM86) to afford compound 1.579 (200 mg, 319.76 μmol, 26.1% yield, HCl salt) as a yellow oil.

LCMS (AM3): rt=0.822 min, (589.3 [M+H]⁺), 100% purity.

### Synthesis of Intermediate 1.585

### (S)-Benzyl 3-(3-((tert-butoxycarbonyl)amino)propoxy)pyrrolidine-1-carboxylate 1.581

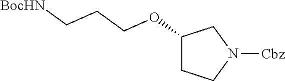 [SMILES: CC(C)(C)OC(=O)NCCCO[C@H]1CCN(C(=O)OCc2ccccc2)C1]

To a solution of(S)-benzyl 3-hydroxypyrrolidine-1-carboxylate (CAS 100858-32-0, 1 g, 4.52 mmol) in DMF (15 mL) was added NaH (198.85 mg, 4.97 mmol, 60% wt.) slowly at 0° C. After being stirred for 0.5 h, tert-butyl (3-bromopropyl)carbamate (1.18 g, 4.97 mmol) was added. The reaction mixture was stirred at 0° C. for 3 h. The mixture was diluted with iced H₂O (50 mL) slowly and extracted with EA (80 mL×2). The combined organic phase was washed (brine, 100 mL), dried (Na₂SO₄), filtered and concentrated in vacuo to give compound 1.581 (900 mg, 2.28 mmol, 52.6% yield) as a colourless oil, which was used directly without purification.

### (S)-Tert-butyl (3-(pyrrolidin-3-yloxy)propyl)carbamate 1.582

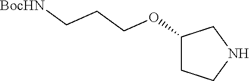 [SMILES: CC(C)(C)OC(=O)NCCCO[C@H]1CCNC1]

To a solution of compound 1.581 (900 mg, 2.28 mmol) in MeOH (8 mL) was added 10% Pd/C (100 mg) at 20° C. The resulting suspension was hydrogenated under one atmosphere H₂ pressure at 20° C. for 12 h. The catalyst was removed by filtration and the filtrate was concentrated in vacuo to afford compound 1.582 (560 mg, 2.29 mmol, 96.38% yield) as a colourless oil, which was used directly without purification.

### (S)-Methyl 5-(3-(3-((tert-butoxycarbonyl)amino)propoxy)pyrrolidin-1-yl)benzo[c][2,6]naphthyridine-8-carboxylate 1.583

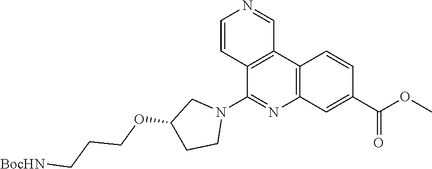 [SMILES: COC(=O)c1ccc2c(c1)nc(N1CC[C@H](OCCCNC(=O)OC(C)(C)C)C1)c1ccncc12]

A mixture of compound 1.582 (560 mg, 2.29 mmol), DIPEA (592.43 mg, 4.58 mmol) and compound 1.1 (624.99 mg, 2.29 mmol) in DMSO (5 mL) was stirred at 80° C. for 12 h. The reaction mixture was diluted with H₂O (50 mL) and then extracted with EA (100 ml×2). The combined organic phase was washed (brine, 80 mL), dried (Na₂SO₄), filtered and concentrated in vacuo to afford compound 1.583 (800 mg, 1.66 mmol, 72.63% yield) as a brown oil, which was used directly without purification.

LCMS (AM3): rt=0.841 min, (481.3 [M+H]⁺), 76.2% purity.

### (S)-Methyl 5-(3-(3-aminopropoxy)pyrrolidin-1-yl)benzo[c][2,6]naphthyridine-8-carboxylate 1.584

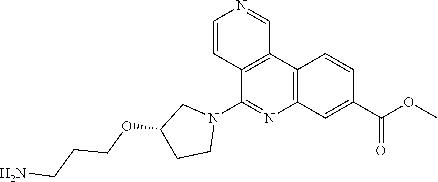 [SMILES: COC(=O)c1ccc2c(c1)nc(N1CC[C@H](OCCCN)C1)c1ccncc12]

To a solution of compound 1.583 (800 mg, 1.66 mmol) in MeOH (5 mL) was added a solution of HCl in MeOH (10 mL, 4 M) at 20° C. The reaction mixture was stirred at 20° C. for 1 h. The reaction mixture was concentrated in vacuo to afford compound 1.584 (800 mg) as a brown solid, which was used directly without purification.

LCMS (AM5): rt=0.800 min, (381.3 [M+H]⁺), 82.8% purity.

### (S)-Methyl 5-(3-(3-((3-chloro-4-(trifluoromethoxy)benzyl)amino)propoxy)pyrrolidin-1-yl)benzo[c][2,6]naphthyridine-8-carboxylate 1.585

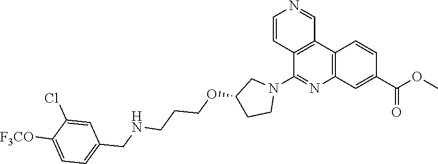 [SMILES: COC(=O)c1ccc2c(c1)nc(N1CC[C@H](OCCCNCc3ccc(OC(F)(F)F)c(Cl)c3)C1)c1ccncc12]

A mixture of compound 1.584 (800 mg, 1.63 mmol, 3×HCl salt), DIPEA (633.26 mg, 4.90 mmol) and 3-chloro-4-(trifluoromethoxy)benzaldehyde (CAS 83279-39-4, 385.11 mg, 1.71 mmol) in MeOH (15 mL) was stirred at 20° C. for 12 h, then NaBH(OAc)₃ (1.04 g, 4.90 mmol) was added. The reaction mixture was stirred at 20° C. for 3 h. The reaction mixture was filtered and the filtrate was purified (PM87) to afford compound 1.585 (260 mg, 415.69 μmol, 25.6% yield, HCl salt) as a yellow oil.

LCMS (AM3): rt=0.825 min, (589.3 [M+H]⁺), 100% purity.

### Synthesis of Intermediate 1.594

### 2-(6-Nitro-1H-benzo[d]imidazol-2-yl)ethanamine 1.591

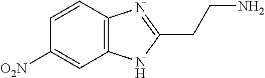 [SMILES: NCCc1nc2ccc([N+](=O)[O-])cc2[nH]1]

A mixture of 4-nitrobenzene-1,2-diamine (4.5 g, 29.39 mmol) and 3-aminopropanoic acid (4.50 g, 50.51 mmol) in aq. HCl solution (50 mL, 6 M) was stirred at 100° C. for 12 h. The mixture was cooled to RT and basified to pH 8 by adding saturated aq. Na₂CO₃ solution. The mixture was filtered and the filtrate was concentrated in vacuo. The residue was purified (PM61) to afford compound 1.591 (3 g, 14.55 mmol, 49.5% yield) as a yellow solid.

LCMS (AM8): rt=0.799 min, (207.1 [M+H]⁺), 86.4% purity.

### Methyl 5-((2-(6-nitro-1H-benzo[d]imidazol-2-yl)ethyl)amino)benzo[c][2,6]naphthyridine-8-carboxylate 1.592

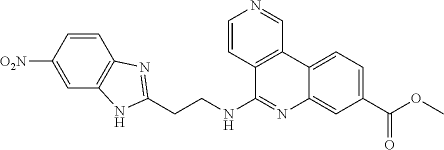 [SMILES: COC(=O)c1ccc2c(c1)nc(NCCc1nc3ccc([N+](=O)[O-])cc3[nH]1)c1ccncc12]

To a mixture of compound 1.591 (500.00 mg, 2.42 mmol) and compound 1.1 (500 mg, 1.83 mmol) in DMSO (6 mL) was added DIPEA (710.93 mg, 5.50 mmol). The mixture was heated to 70° C. and stirred for 12 h. The mixture was poured into H₂O (100 mL) and the precipitate was collected by filtration. The filter cake was washed with H₂O (20 mL×3) and dried in vacuo to afford compound 1.592 (425 mg) as a brown solid, which was used directly without purification.

LCMS (AM3): rt=0.804 min, (443.1 [M+H]⁺), 72.4% purity.

### Methyl 5-((2-(6-amino-1H-benzo[d]imidazol-2-yl)ethyl)amino)benzo[c][2,6]naphthyridine-8-carboxylate 1.593

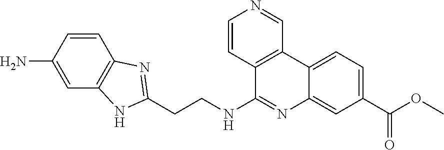 [SMILES: COC(=O)c1ccc2c(c1)nc(NCCc1nc3ccc(N)cc3[nH]1)c1ccncc12]

To a mixture of compound 1.592 (750 mg, 1.70 mmol) in EtOH (10 mL) and H₂O (2 mL) was added iron powder (946.68 mg, 16.95 mmol) and NH₄Cl (906.78 mg, 16.95 mmol). The mixture was then heated to 60° C. and stirred for 1 h. The mixture was diluted with H₂O (60 mL) and filtered. The filtrate was extracted with EA (40 mL×3) and the combined organic phase was washed (brine, 100 mL), dried (Na₂SO₄), filtered and concentrated in vacuo to afford compound 1.593 (80 mg, 193.97 μmol, 11.4% yield) as a yellow solid.

LCMS (AM3): rt=0.724 min, (413.2 [M+H]⁺), 95.3% purity.

### Methyl 5-((2-(6-(((2-chloro-[1,1′-biphenyl]-4-yl)methyl)amino)-1H-benzo[d]imidazol-2-yl)ethyl)amino)benzo[c][2,6]naphthyridine-8-carboxylate 1.594

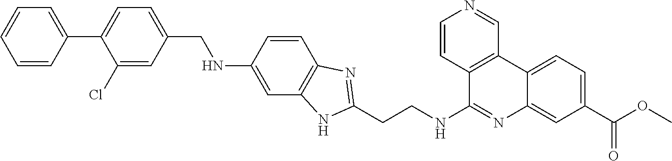 [SMILES: COC(=O)c1ccc2c(c1)nc(NCCc1nc3ccc(NCc4ccc(-c5ccccc5)c(Cl)c4)cc3[nH]1)c1ccncc12]

A mixture of 3-chloro-4-phenylbenzaldehyde (CAS 57592-44-6, 63.04 mg, 290.95 μmol) and compound 1.593 (60 mg, 145.47 μmol) in MeOH (4 mL) was stirred at 30° C. for 12 h, then NaBH₃CN (91.42 mg, 1.45 mmol) was added. The mixture was stirred at 30° C. for 2 h. The mixture was concentrated in vacuo and the residue was purified (PM61) to afford compound 1.594 (40 mg, 63.28 μmol, 43.5% yield) as a brown solid.

LCMS (AM3): rt=0.860 min, (613.1 [M+H]⁺), 97.4% purity.

### Synthesis of Intermediate 1.597

### Methyl 5-((2-((3-(((tert-butoxycarbonyl)amino)methyl)cyclobutyl)amino)ethyl)amino)benzo[c][2,6]naphthyridi ne-8-carboxylate 1.595

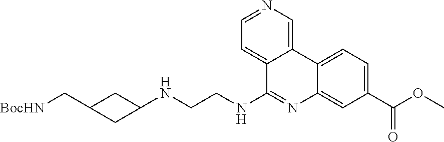 [SMILES: COC(=O)c1ccc2c(c1)nc(NCCNC1CC(CNC(=O)OC(C)(C)C)C1)c1ccncc12]

A mixture of Intermediate A (500 mg, 1.50 mmol), DIPEA (388.37 mg, 3.00 mmol) and tert-butyl ((3-oxocyclobutyl)methyl)carbamate (299.36 mg, 1.50 mmol) in MeOH (10 mL) was stirred for 0.5 h, then NaBH₃CN (944.19 mg, 15.02 mmol) was added. The mixture was stirred at 30° C. for 11.5 h. The mixture was concentrated in vacuo and purified (PM88) to afford compound 1.595 (300 mg, 625.56 μmol, 41.6% yield) as a yellow solid.

LCMS (AM3): rt=0.830 min, (480.2 [M+H]⁺), 96.4% purity.

### Methyl 5-((2-((3-(aminomethyl)cyclobutyl)amino)ethyl)amino)benzo[c][2,6]naphthyridine-8-carboxylate 1.596

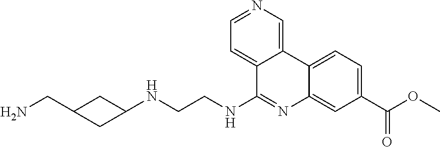 [SMILES: COC(=O)c1ccc2c(c1)nc(NCCNC1CC(CN)C1)c1ccncc12]

A mixture of compound 1.595 (250 mg, 521.30 μmol) in a solution of HCl in MeOH (10.00 mL, 4 M) was stirred at 30° C. for 0.5 h The mixture was concentrated in vacuo to afford compound 1.596 (220 mg, HCl salt) as a yellow solid, which was used directly without purification.

LCMS (AM3): rt=0.705 min, (380.2 [M+H]⁺), 97.7% purity.

### Methyl 5-((2-((3-(((3-chloro-4-(trifluoromethoxy)benzyl)amino)methyl)cyclobutyl)amino)ethyl)amino)benzo[c][2,6]na phthyridine-8-carboxylate 1.597

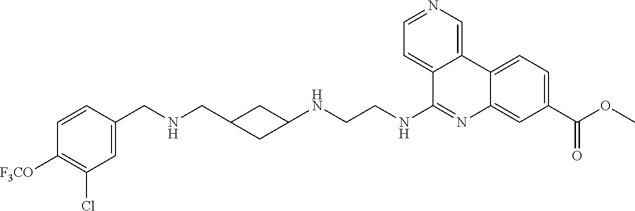 [SMILES: COC(=O)c1ccc2c(c1)nc(NCCNC1CC(CNCc3ccc(OC(F)(F)F)c(Cl)c3)C1)c1ccncc12]

A mixture of compound 1.596 (220 mg, 528.95 μmol, HCl salt), DIPEA (136.73 mg, 1.06 mmol) and 3-chloro-4-(trifluoromethoxy)benzaldehyde (CAS 83279-39-4, 118.78 mg, 528.95 μmol) in MeOH (5 mL) was stirred at 30° C. for 11 h, then NaBH₃CN (332.41 mg, 5.29 mmol) was added. The mixture was stirred at 30° C. for 1 h. The mixture was concentrated in vacuo and the residue was purified (PM89) to afford compound 1.597 (150 mg) as a yellow solid.

LCMS (AM3): rt=0.810 min, (588.3 [M+H]⁺), 59.5% purity.

### Synthesis of Intermediate 1.603

### Benzyl 4-(3-((tert-butoxycarbonyl)amino)propoxy)piperidine-1-carboxylate 1.599

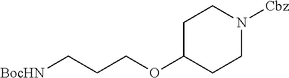 [SMILES: CC(C)(C)OC(=O)NCCCOC1CCN(C(=O)OCc2ccccc2)CC1]

To a mixture of benzyl 4-hydroxypiperidine-1-carboxylate (CAS 95798-23-5, 5 g, 21.25 mmol) in DMF (50 mL) was added NaH (934.97 mg, 23.38 mmol, 60% wt.) at 0° C. The reaction mixture was stirred for 0.5 h then tert-butyl (3-bromopropyl)carbamate (6.07 g, 25.50 mmol) was added and the mixture was warmed to 20° C. and stirred for 3 h. The mixture was quenched with H₂O (60 mL) and the aq. phase was extracted with EA (60 mL×2). The combined organic phase was washed (brine, 100 mL×3), dried (Na₂SO₄), filtered and concentrated in vacuo. The residue was purified (PM2) to afford compound 1.599 (4 g, 10.19 mmol, 47.9% yield) as a colourless oil.

¹H NMR (400 MHz, CHCl₃-d) δ: 7.39-7.29 (m, 5H), 5.13 (s, 2H), 4.88 (br s, 1H), 3.80-3.70 (m, 2H), 3.52 (t, J=8.0 Hz, 2H), 3.50-3.44 (m, 1H), 3.30-3.17 (m, 4H), 1.87-1.73 (m, 4H), 1.65-1.52 (m, 2H), 1.44 (s, 9H) ppm.

### Tert-butyl (3-(piperidin-4-yloxy)propyl)carbamate 1.600

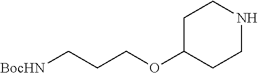 [SMILES: CC(C)(C)OC(=O)NCCCOC1CCNCC1]

To a solution of compound 1.599 (1.7 g, 4.33 mmol) in MeOH (10 mL) was added 10% Pd/C (1 g, 4.33 mmol) under N₂. The resulting suspension was degassed under vacuum and purged with hydrogen three times. The mixture was hydrogenated under one atmosphere H₂ pressure at 20° C. for 12 h. The catalyst was removed by filtration and the filtrate was concentrated in vacuo to afford compound 1.600 (1.2 g) as a colourless oil.

LCMS (AM3): rt=0.871 min, (203.2 [M-tBu+2H]⁺), 100% purity.

### Methyl 5-(4-(3-((tert-butoxycarbonyl)amino)propoxy)piperidin-1-yl)benzo[c][2,6]naphthyridine-8-carboxylate 1.601

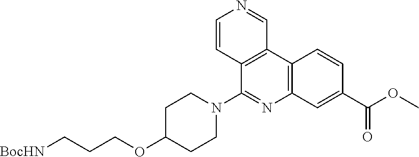 [SMILES: COC(=O)c1ccc2c(c1)nc(N1CCC(OCCCNC(=O)OC(C)(C)C)CC1)c1ccncc12]

To a mixture of compound 1.600 (500 mg, 1.94 mmol) and compound 1.1 (500 mg, 1.83 mmol) in DMSO (8 mL) was added DIPEA (473.96 mg, 3.67 mmol). The mixture was heated to 80° C. and stirred for 2 h. The mixture was cooled to RT and diluted with H₂O (50 mL). The mixture was extracted with EA (50 mL×2). The combined organic phase was washed (brine, 50 mL), dried (Na₂SO₄) and concentrated in vacuo. The residue was purified (PM40) to afford compound 1.601 (800 mg, 1.62 mmol, 88.2% yield) as a yellow solid.

LCMS (AM3): rt=0.985 min, (495.3 [M+H]⁺), 85.5% purity.

### Methyl 5-(4-(3-aminopropoxy)piperidin-1-yl)benzo[c][2,6]naphthyridine-8-carboxylate 1.602

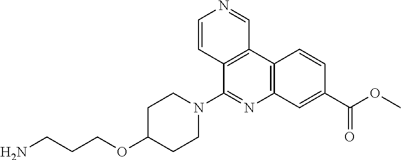 [SMILES: COC(=O)c1ccc2c(c1)nc(N1CCC(OCCCN)CC1)c1ccncc12]

To a mixture of compound 1.601 (500 mg, 1.01 mmol) in MeOH (10 mL) was added a solution of HCl in 1,4-dioxane (8 mL, 4 M) at 20° C. The mixture was stirred at 20° C. for 1.5 h. The mixture was concentrated in vacuo to afford compound 1.602 (450 mg, HCl salt) as a yellow solid, which was used directly without purification.

LCMS (AM3): rt=0.778 min, (395.3 [M+H]⁺), 61.7% purity.

### Methyl 5-(4-(3-((3-chloro-4-(trifluoromethoxy)benzyl)amino)propoxy)piperidin-1-yl)benzo[c][2,6]naphthyridine-8-carboxylate 1.603

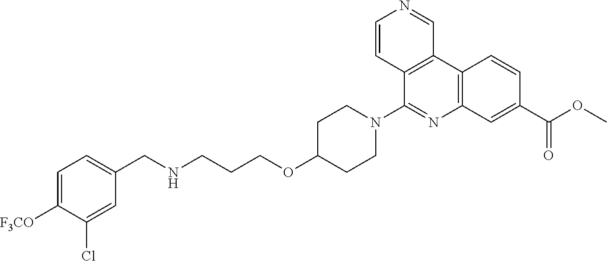 [SMILES: COC(=O)c1ccc2c(c1)nc(N1CCC(OCCCNCc3ccc(OC(F)(F)F)c(Cl)c3)CC1)c1ccncc12]

A mixture of compound 1.602 (200 mg, 464.12 μmol, HCl salt), DIPEA (179.95 mg, 1.39 mmol) and 3-chloro-4-(trifluoromethoxy)benzaldehyde (104.22 mg, 464.12 μmol) in MeOH (3 mL) was stirred at 35° C. for 12 h, then NaBH₃CN (590.19 mg, 2.78 mmol) was added. The mixture was stirred at 35° C. for 0.5 h. The mixture was concentrated in vacuo and the residue was purified (PM90) to afford compound 1.603 (100 mg, 165.83 μmol, 35.7% yield) as a yellow oil.

LCMS (AM3): rt=0.903 min, (603.2 [M+H]⁺), 98.6% purity.

### Synthesis of Intermediate 1.616

### Methyl 3-(((2-chloro-[1,1′-biphenyl]-4-yl)methyl)amino)cyclobutanecarboxylate 1.611

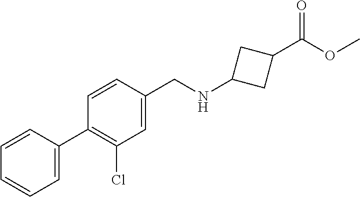 [SMILES: COC(=O)C1CC(NCc2ccc(-c3ccccc3)c(Cl)c2)C1]

A solution of (2-chloro-[1,1′-biphenyl]-4-yl)methanamine (ref: Bioorganic and Medicinal Chemistry, 2017, 25 (13), 3471-3482, 8 g, 36.75 mmol) and methyl 3-oxocyclobutanecarboxylate (CAS 695-95-4, 4.71 g, 36.75 mmol) in MeOH (20 mL) was stirred at 25° C. for 12 h, then NaBH₃CN (2.31 g, 36.75 mmol) was added. The reaction mixture was stirred at 25° C. for 2 h. The mixture was filtered, and the filtrate was concentrated in vacuo. The residue was purified (PM46) to afford compound 1.611 (4 g, 28.9% yield) as a pale oil.

LCMS (AM3): rt=0.817 min, 330.2 [M+H]⁺, 21.4% purity.

### Methyl 3-((tert-butoxycarbonyl) ((2-chloro-[1,1′-biphenyl]-4-yl)methyl)amino)cyclobutanecarboxylate 1.612

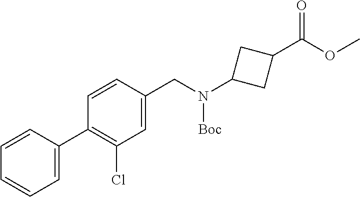 [SMILES: COC(=O)C1CC(N(Cc2ccc(-c3ccccc3)c(Cl)c2)C(=O)OC(C)(C)C)C1]

A solution of compound 1.611 (2.3 g, 6.97 mmol), (Boc)₂O (2.28 g, 10.46 mmol) and NaHCO₃ (1.17 g, 13.95 mmol) in THF (15 ml) was stirred at 25° C. for 2 h. The mixture was diluted with H₂O (15 mL) and extracted with EA (20 mL×3). The combined organic phase was dried with Na₂SO₄ and concentrated in vacuo to afford compound 1.612 (3.5 g, 99.8% yield) as a colourless oil, which was used directly without further purification.

LCMS (AM3): rt=0.980 min, 330.0 [M−tBuCO2+2H]⁺, 77.6% purity.

### Tert-butyl ((2-chloro-[1,1′-biphenyl]-4-yl)methyl)(3-(hydroxymethyl)cyclobutyl)carbamate 1.613

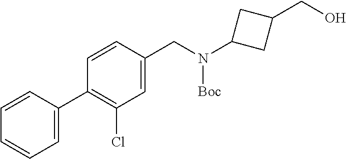 [SMILES: CC(C)(C)OC(=O)N(Cc1ccc(-c2ccccc2)c(Cl)c1)C1CC(CO)C1]

To a three-neck flask was added LAH (264.84 mg, 6.98 mmol) and THF (20 mL), and the mixture was cooled to 0° C. The reaction mixture was degassed with N₂. A solution of compound 1.612 (2 g, 4.65 mmol) in THF (20 mL) was added dropwise and the mixture was stirred at 0° C. for 1 h. The mixture was quenched with EA (20 mL) and H₂O (20 mL), Na₂SO₄ (20 g) was added. The mixture was filtered and the filtrate was concentrated in vacuo. The residue was purified (PM3) to afford compound 1.613 (1.35 g, 72.2% yield, 100% purity) as a colourless oil.

LCMS (AM3): rt=0.957 min, 402.2 [M+H]⁺, 98.8% purity.

### Tert-butyl ((2-chloro-[1,1′-biphenyl]-4-yl)methyl)(3-formylcyclobutyl)carbamate 1.614

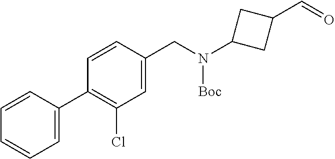 [SMILES: CC(C)(C)OC(=O)N(Cc1ccc(-c2ccccc2)c(Cl)c1)C1CC(C=O)C1]

A mixture of compound 1.613 (550 mg, 1.37 mmol) and DMP (715 mg, 1.69 mmol) in DCM (15 mL) was stirred at RT for 4 h. The reaction mixture was concentrated in vacuo and purified (PM8) to afford compound 1.614 (290 mg, 48.8% yield) as a colourless oil.

LCMS (AM3): rt=1.074 min, (422.3 [M+Na]⁺), 92.0% purity.

### Methyl 5-((2-(((3-((tert-butoxycarbonyl) ((2-chloro-[1,1′-biphenyl]-4-yl)methyl)amino)cyclobutyl)methyl)amino)ethyl)amino)benzo[c][2,6]naphthyridine-8-carboxylate 1.615

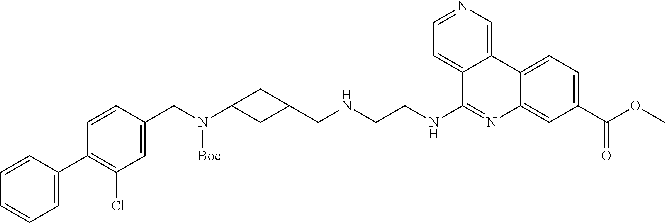 [SMILES: COC(=O)c1ccc2c(c1)nc(NCCNCC1CC(N(Cc3ccc(-c4ccccc4)c(Cl)c3)C(=O)OC(C)(C)C)C1)c1ccncc12]

A mixture of compound 1.614 (270 mg, 0.675 mmol), Intermediate A (405 mg, 1.22 mmol, HCl salt) and DIPEA (1.55 mmol, 0.27 mL) in MeOH (30 mL) was stirred at RT for 1 h, then NaBH(OAc)₃ (572 mg, 2.70 mmol) was added. The reaction mixture was then stirred at RT for 1 h. The reaction mixture was concentrated in vacuo and the residue was purified (PM91) to afford compound 1.615 (230 mg, 50.1% yield) as a white solid.

LCMS (AM3): rt=0.990 min, (680.3 [M+H]⁺), 99.4% purity.

### Tert-butyl (3-(((2-((8-carbamoylbenzo[c][2,6]naphthyridin-5-yl)amino)ethyl)amino)methyl)cyclobutyl)((2-chloro-[1,1′-biphenyl]-4-yl)methyl)carbamate 1.616

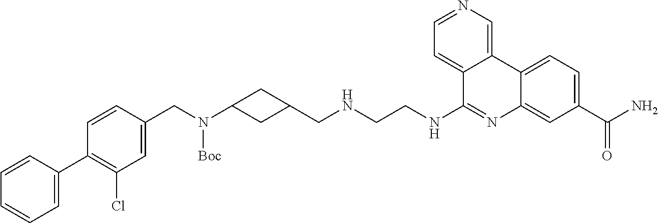 [SMILES: CC(C)(C)OC(=O)N(Cc1ccc(-c2ccccc2)c(Cl)c1)C1CC(CNCCNc2nc3cc(C(N)=O)ccc3c3cnccc23)C1]

A mixture of compound 1.615 (230 mg, 0.338 mmol) in a solution of NH₃ in MeOH (15 mL, 7 M) was stirred in a sealed tube at 90° C. for 16 h. The reaction mixture was concentrated in vacuo to afford compound 1.616 (260 mg) as a colourless oil, which was used directly without purification.

LCMS (AM1): rt=0.810 min, (665.3 [M+H]⁺), 30.5% purity.

### Synthesis of Intermediate 1.620

### Methyl 5-(piperazin-1-yl)benzo[c][2,6]naphthyridine-8-carboxylate 1.617

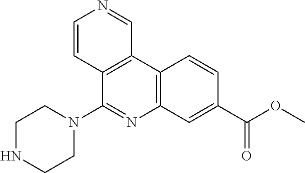 [SMILES: COC(=O)c1ccc2c(c1)nc(N1CCNCC1)c1ccncc12]

A solution of compound 1.1 (600 mg, 1.76 mmol), piperazine (1.52 g, 17.60 mmol) and TEA (1.45 g, 14.37 mmol) in DMSO (6 mL) was stirred at 80° C. for 1 h. The mixture was poured into H₂O and extracted with EA (50 mL×2). The combined organic phase was washed (brine, 100 mL), dried (Na₂SO₄) and concentrated in vacuo. The residue was purified (PM48) to afford compound 1.617 (640 mg, HCl salt) as a brown solid.

LCMS (AM3): rt=0.734 min, (323.2 [M+H]⁺), 81.3% purity.

### Methyl 5-(4-(4-(((benzyloxy)carbonyl)amino)butyl)piperazin-1-yl)benzo[c][2,6]naphthyridine-8-carboxylate 1.618

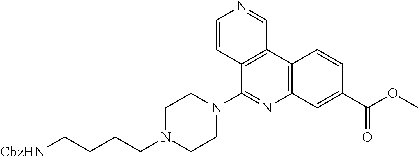 [SMILES: COC(=O)c1ccc2c(c1)nc(N1CCN(CCCCNC(=O)OCc3ccccc3)CC1)c1ccncc12]

A solution of compound 1.617 (590 mg, 1.64 mmol, HCl salt), DIPEA (847.81 mg, 6.56 mmol) and benzyl (4-oxobutyl)carbamate (1.09 g, 4.92 mmol) in MeOH (15 mL) was stirred at 20° C. for 12 h, then NaBH(OAc)₃ (1.74 g, 8.20 mmol) was added. The reaction mixture was stirred at 20° C. for 1 h. The mixture was filtered and the filtrate was concentrated in vacuo. The residue was purified (PM46) to afford compound 1.618 (700 mg, 662.44 μmol, FA salt) as a brown gum.

LCMS (AM3): rt=0.671 min, (528.1 [M+H]⁺), 51.0% purity.

### Methyl 5-(4-(4-aminobutyl)piperazin-1-yl)benzo[c][2,6]naphthyridine-8-carboxylate 1.619

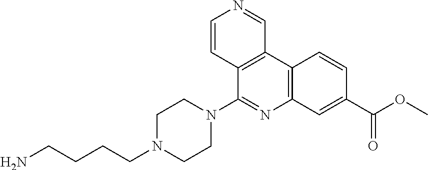 [SMILES: COC(=O)c1ccc2c(c1)nc(N1CCN(CCCCN)CC1)c1ccncc12]

To a solution of compound 1.618 (380 mg, 662.44 μmol, FA salt) in MeOH (20 mL) was added aq. ammonium hydroxide solution (5 mL, 38.95 mmol, 30%) followed by 10% Pd/C (100 mg, 10%) under N₂ at 20° C. The reaction vessel was with H₂ (×3), then hydrogenated under one atmosphere H₂ at 20° C. for 4 h. The catalyst was removed by filtration and the filtrate was concentrated in vacuo to afford compound 1.619 (200 mg, 208.28 μmol, 76.7% yield) as a yellow gum.

LCMS (AM3): rt=0.651 min, (394.2 [M+H]⁺), 90.9% purity.

### Methyl 5-(4-(4-((3-chloro-4-(trifluoromethoxy)benzyl)amino)butyl)piperazin-1-yl)benzo[c][2,6]naphthyridine-8-carboxylate 1.620

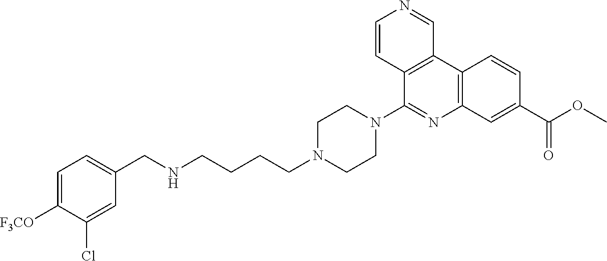 [SMILES: COC(=O)c1ccc2c(c1)nc(N1CCN(CCCCNCc3ccc(OC(F)(F)F)c(Cl)c3)CC1)c1ccncc12]

A mixture of compound 1.619 (190 mg, 482.87 μmol) and 3-chloro-4-(trifluoromethoxy)benzaldehyde (CAS 83279-39-4, 97 mg, 431.95 μmol) in MeOH (5 mL) was stirred at 20° C. for 12 h, then NaBH(OAc)₃ (409.36 mg, 1.93 mmol) was added. The reaction mixture was stirred at 20° C. for 1 h. The mixture was filtered and the filtrate was concentrated in vacuo. The residue was purified (PM92) to afford compound 1.620 (100 mg, 154.30 μmol, 31.9% yield, FA salt) as a light yellow solid.

LCMS (AM3): rt=0.653 min, (602.0 [M+H]⁺), 51.4% purity.

### Synthesis of Intermediate 1.628

### Methyl 5-((4-(2-aminoethyl)phenethyl)amino)benzo[c][2,6]naphthyridine-8-carboxylate 1.627

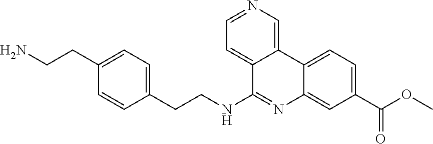 [SMILES: COC(=O)c1ccc2c(c1)nc(NCCc1ccc(CCN)cc1)c1ccncc12]

A solution of compound 1.1 (195 mg, 715.11 μmol), 2,2′-(1,4-phenylene) diethanamine (ref: US2003229102, 352.36 mg, 2.15 mmol) and DIPEA (277.27 mg, 2.15 mmol) in DMSO (4 mL) was stirred at 70° C. for 12 h. The reaction mixture was poured into H₂O (70 mL) and extracted with EA (40 mL×3). The combined organic phase was washed (brine, 70 mL×3), dried (Na₂SO₄), filtered and concentrated in vacuo. The residue was purified (PM48) to afford compound 1.627 (219 mg, 481.17 μmol, 67.3% yield, HCl salt) as a yellow solid.

LCMS (AM3): rt=0.742 min, (401.2 [M+H]⁺), 96.6% purity.

### Methyl 5-((4-(2-(((2-chloro-[1,1′-biphenyl]-4-yl)methyl)amino)ethyl)phenethyl)amino)benzo[c][2,6]naphthyridine-8-carboxylate 1.628

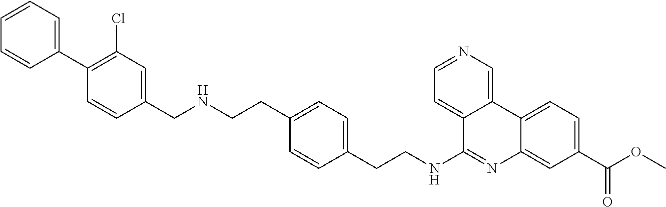 [SMILES: COC(=O)c1ccc2c(c1)nc(NCCc1ccc(CCNCc3ccc(-c4ccccc4)c(Cl)c3)cc1)c1ccncc12]

A solution of compound 1.627 (185 mg, 423.41 μmol), DIPEA (109.44 mg, 846.81 μmol) and 3-chloro-4-phenylbenzaldehyde (CAS 57592-44-6, 96.32 mg, 444.58 μmol) in MeOH (3 mL) was stirred at 25° C. for 1 h, then NaBH(OAc)₃ (89.74 mg, 423.41 μmol) was added. The mixture was stirred at 25° C. for 11 h. The mixture was concentrated in vacuo and the residue was purified (PM93) to afford compound 1.628 (93 mg, 154.71 μmol, 36.5% yield, HCl salt) as a light yellow solid.

LCMS (AM3): rt=0.759 min, (601.1 [M+H]⁺), 35.2% purity.

### Synthesis of Intermediate 1.641

### (S)-Tert-butyl (5-(cyanomethoxy)pentan-2-yl)carbamate 1.637

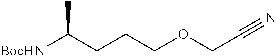 [SMILES: C[C@@H](CCCOCC#N)NC(=O)OC(C)(C)C]

To a solution of(S)-tert-butyl (5-hydroxypentan-2-yl)carbamate (CAS 104700-48-3, 2.7 g, 13.28 mmol) in DCM (30 mL) was added TBAI (981.21 mg, 2.66 mmol), 2-bromoacetonitrile (3.19 g, 26.56 mmol) and Ag₂O (3.08 g, 13.28 mmol) at 20° C. The reaction mixture was stirred at 20° C. for 12 h. The reaction mixture was filtered and the filtrate was concentrated in vacuo. The residue was purified (PM10) to afford compound 1.637 (650 mg, 2.68 mmol, 20.2% yield) as a colourless oil.

¹H NMR (400 MHz, CHCl₃-d) δ: 4.46-4.42 (m, 1H), 4.22 (s, 2H), 3.57 (t, J=8.0 Hz, 2H), 1.70-1.40 (m, 4H), 1.42 (s, 9H), 1.11 (d, J=6.8 Hz, 3H) ppm.

### (S)-Tert-butyl (5-(2-aminoethoxy)pentan-2-yl)carbamate 1.638

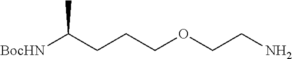 [SMILES: C[C@@H](CCCOCCN)NC(=O)OC(C)(C)C]

To a solution of compound 1.637 (650 mg, 2.68 mmol) in MeOH (15 mL) was added aq. ammonium hydroxide solution (2.73 g, 30%) and Raney Ni (100 mg) under N₂. The reaction mixture was purged with hydrogen and hydrogenated under three atmospheres' pressure at 20° C. for 12 h. The reaction mixture was filtered and the filtrate was concentrated in vacuo to afford compound 1.638 (650 mg) as a colourless oil, which was used directly without purification.

### (S)-Methyl 5-((2-((4-((tert-butoxycarbonyl)amino)pentyl)oxy)ethyl)amino)benzo[c][2,6]naphthyridine-8-carboxylate 1.639

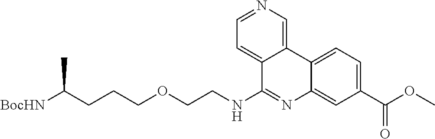 [SMILES: COC(=O)c1ccc2c(c1)nc(NCCOCCC[C@H](C)NC(=O)OC(C)(C)C)c1ccncc12]

A mixture of compound 1.638 (600 mg, 2.44 mmol), compound 1.1 (664.16 mg, 2.44 mmol) and DIPEA (629.57 mg, 4.87 mmol) in DMSO (20 mL) was stirred at 70° C. for 12 h. The reaction mixture was diluted with H₂O (60 mL) and extracted with EA (80 mL×2). The combined organic phase was washed (brine, 100 mL), dried (Na₂SO₄), filtered and concentrated in vacuo to afford compound 1.639 (1.2 g) as a brown oil, which was used directly without further purification.

LCMS (AM3): rt=0.852 min, (483.3 [M+H]⁺), 59.0% purity.

### (S)-Methyl 5-((2-((4-aminopentyl)oxy)ethyl)amino)benzo[c][2,6]naphthyridine-8-carboxylate 1.640

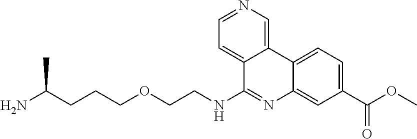 [SMILES: COC(=O)c1ccc2c(c1)nc(NCCOCCC[C@H](C)N)c1ccncc12]

To a solution of compound 1.639 (600 mg, 1.24 mmol) in 1,4-dioxane (5 mL) was added a solution of HCl in MeOH (15 mL, 4 M) at 20° C. The reaction mixture was stirred at 20° C. for 2 h. The reaction mixture was concentrated in vacuo to afford compound 1.640 (450 mg, 764.80 μmol, 94.6% yield) as a brown oil, which was used directly without purification.

LCMS (AM5): rt=0.817 min, (383.2 [M+H]⁺), 62.5% purity.

### (S)-Methyl 5-((2-((4-((3-chlorobenzyl)amino)pentyl)oxy)ethyl)amino)benzo[c][2,6]naphthyridine-8-carboxylate 1.641

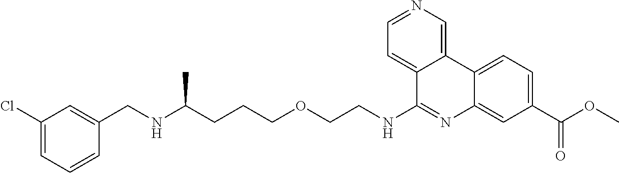 [SMILES: COC(=O)c1ccc2c(c1)nc(NCCOCCC[C@H](C)NCc1cccc(Cl)c1)c1ccncc12]

A mixture of compound 1.640 (450 mg, 764.80 μmol, 65% purity), DIPEA (98.84 mg, 764.80 μmol) and 3-chlorobenzaldehyde (107.50 mg, 764.80 μmol) in MeOH (10 mL) was stirred at 20° C. for 12 h, then NaBH(OAc)₃ (162.09 mg, 764.80 μmol) was added. The reaction mixture was stirred at 20° C. for 3 h. The reaction mixture was filtered, diluted with H₂O (40 mL) and extracted with EA (60 mL×2). The combined organic phase was washed (brine, 80 mL), dried (Na₂SO₄), filtered and concentrated in vacuo. The residue was purified (PM94) to afford compound 1.641 (100 mg, 161.02 μmol, 21.1% yield, TFA salt) as a yellow solid.

LCMS (AM3): rt=0.782 min, (507.3 [M+H]⁺), 54.2% purity.

### Synthesis of Intermediate 1.659

### 4-((8-(Methoxycarbonyl)benzo[c][2,6]naphthyridin-5-yl)amino)butanoic acid 1.656

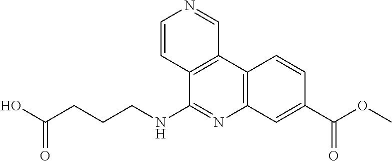 [SMILES: COC(=O)c1ccc2c(c1)nc(NCCCC(=O)O)c1ccncc12]

A mixture of 4-aminobutanoic acid (2.27 g, 22.00 mmol) and compound 1.1 (1 g, 3.67 mmol) and DIPEA (4.27 g, 33.00 mmol) in DMSO (10 mL) was heated to 80° C. for 12 h. The reaction mixture was diluted with H₂O (100 mL) and the precipitate was collected by filtration. The filter cake was dried under vacuum to afford compound 1.656 (800 mg, 64.3% yield) as a brown solid, which was used directly without further purification.

LCMS (AM3): rt=0.737 min, (340.5 [M+H]⁺), 80.8% purity.

### Methyl 5-((4-((2-((tert-butoxycarbonyl)amino)ethyl)amino)-4-oxobutyl)amino)benzo[c][2,6]naphthyridine-8-carboxylate 1.657

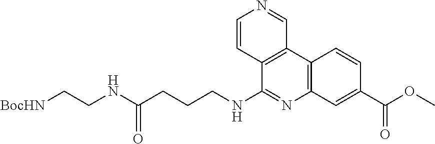 [SMILES: COC(=O)c1ccc2c(c1)nc(NCCCC(=O)NCCNC(=O)OC(C)(C)C)c1ccncc12]

To a solution of compound 1.656 (378 mg, 1.11 mmol) and HATU (633.08 mg, 1.67 mmol) in DMF (10 mL) was added DIPEA (215.19 mg, 1.67 mmol) at 20° C. After stirring at 20° C. for 0.5 h, tert-butyl (2-aminoethyl)carbamate (213.41 mg, 1.33 mmol) was added. The mixture was stirred at 20° C. for 2 h. The reaction mixture was diluted with H₂O (100 mL) and extracted with EA (50 mL×3). The combined organic layer was washed by brine (100 mL×2), dried (Na₂SO₄), filtered and concentrated in vacuo. The residue was purified (PM46) to afford compound 1.657 (410 mg, 76.7% yield) as a brown solid.

LCMS (AM3): rt=0.625 min, (482.1 [M+H]⁺), 42.0% purity.

### Methyl 5-((4-((2-aminoethyl)amino)-4-oxobutyl)amino)benzo[c][2,6]naphthyridine-8-carboxylate 1.658

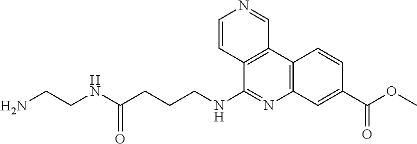 [SMILES: COC(=O)c1ccc2c(c1)nc(NCCCC(=O)NCCN)c1ccncc12]

To a solution compound 1.657 (410 mg, 851.43 μmol) in DCM (5 mL) was added TFA (126.28 mg, 1.11 mmol) at 20° C. The mixture was stirred at 20° C. for 1 h. The mixture was concentrated in vacuo to afford compound 1.658 (730 mg, TFA salt) which was used directly without purification.

LCMS (AM3): rt=0.708 min, (382.5 [M+H]⁺), 26.6% purity.

### Methyl 5-((4-((2-((3-chloro-4-(trifluoromethoxy)benzyl)amino)ethyl)amino)-4-oxobutyl)amino)benzo[c][2,6]naphthyridine-8-carboxylate 1.659

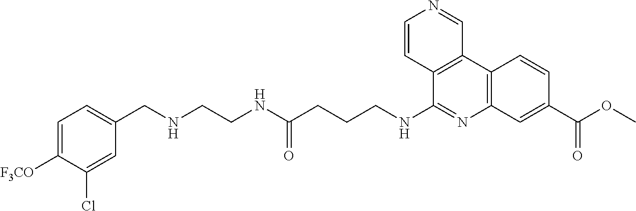 [SMILES: COC(=O)c1ccc2c(c1)nc(NCCCC(=O)NCCNCc1ccc(OC(F)(F)F)c(Cl)c1)c1ccncc12]

A solution of compound 1.658 (730 mg, 1.91 mmol) and 3-chloro-4-(trifluoromethoxy)benzaldehyde (CAS 83279-39-4, 515.74 mg, 2.30 mmol) in MeOH (10 mL) was stirred at 25° C. for 12 h, then NaBH₃CN (120.27 mg, 1.91 mmol) was added and the mixture was stirred at 25° C. for 3 h. The mixture was diluted with MeOH (10 mL) and filtered to remove insoluble material. The filtrate was concentrated in vacuo and purified (PM46) to afford compound 1.659 (90 mg, 8.0% yield) as a yellow oil.

LC-MS (AM3): rt=0.606 min, (590.0 [M+H]⁺), 11.6% purity.

### Synthesis of Intermediate 1.665

### Tert-butyl (3-((2-amino-2-oxoethoxy)methyl)cyclobutyl)((2-chloro-[1,1′-biphenyl]-4-yl)methyl)carbamate 1.662

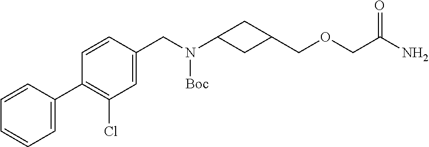 [SMILES: CC(C)(C)OC(=O)N(Cc1ccc(-c2ccccc2)c(Cl)c1)C1CC(COCC(N)=O)C1]

To a solution of compound 1.613 (1 g, 2.49 mmol) in THF (20 mL) was added NaH (497.56 mg, 12.44 mmol, 60% dispersion in oil) at 20° C. slowly, followed by 2-iodoacetamide (460.19 mg, 2.49 mmol). The resulting mixture was heated to 60° C. and stirred for 6 h. The reaction mixture was diluted with H₂O (50 mL) and extracted with EA (50 mL×3). The combined organic layer was washed (brine, 100 mL×2), dried (Na₂SO₄), filtered and concentrated in vacuo. The residue was purified (PM46) to afford compound 1.662 (260 mg, 566.48 μmol, 22.8% yield) as a yellow oil.

LCMS (AM3): rt=1.097 min, (481.2 [M+Na]⁺), 35.1% purity.

### Tert-butyl (3-((2-aminoethoxy)methyl)cyclobutyl)((2-chloro-[1,1′-biphenyl]-4-yl)methyl)carbamate 1.663

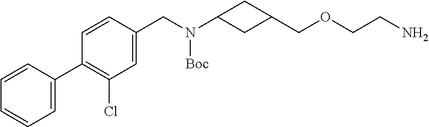 [SMILES: CC(C)(C)OC(=O)N(Cc1ccc(-c2ccccc2)c(Cl)c1)C1CC(COCCN)C1]

To a solution of compound 1.662 (260 mg, 566.48 μmol) in THF (5 mL) was added a solution of borane-THF complex (566.48 μL, 1 M) at 0° C. under N₂. The mixture was stirred at 0° C. for 2 h. The reaction mixture was quenched with aq. NaOH solution (1 N, 20 mL) and extracted with EA (20 mL×3). The combined organic layer was washed (brine, 10 mL×2), dried (Na₂SO₄), filtered and concentrated in vacuo to afford compound 1.663 (220 mg, 494.39 μmol, 87.3% yield) as a yellow oil.

LCMS (AM3): rt=0.915 min, (445.3 [M+H]⁺), 77.7% purity.

### Methyl 5-((2-((3-((tert-butoxycarbonyl) ((2-chloro-[1,1′-biphenyl]-4-yl)methyl)amino)cyclobutyl)methoxy)ethyl)amino)benzo[c][2,6]naphthyridine-8-carboxylate 1.664

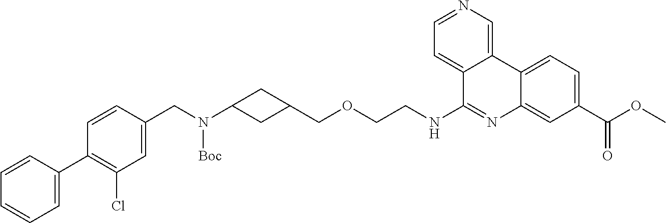 [SMILES: COC(=O)c1ccc2c(c1)nc(NCCOCC1CC(N(Cc3ccc(-c4ccccc4)c(Cl)c3)C(=O)OC(C)(C)C)C1)c1ccncc12]

A mixture of compound 1.663 (170 mg, 382.03 μmol), compound 1.1 (104.17 mg, 382.03 μmol) and DIPEA (98.75 mg, 764.06 μmol) in DMSO (2 mL) was stirred at 70° C. for 12 h. The reaction mixture was diluted with H₂O (30 mL) and extracted with EA (25 mL×2). The combined organic layer was washed (brine, 25 mL×2), dried (Na₂SO₄), filtered and concentrated in vacuo. The residue was purified (PM46) to afford compound 1.664 (55 mg, 80.74 μmol, 21.1% yield) as a light yellow solid.

LCMS (AM3): rt=1.010 min, (681.4 [M+H]⁺), 55.1% purity.

### 5-((2-((3-((Tert-butoxycarbonyl)((2-chloro-[1,1′-biphenyl]-4-yl)methyl)amino)cyclobutyl)methoxy)ethyl)amino)benzo[c][2,6]naphthyridine-8-carboxylic acid 1.665

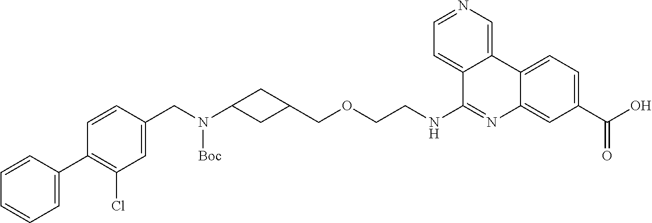 [SMILES: CC(C)(C)OC(=O)N(Cc1ccc(-c2ccccc2)c(Cl)c1)C1CC(COCCNc2nc3cc(C(=O)O)ccc3c3cnccc23)C1]

A mixture of compound 1.664 (55 mg, 80.74 μmol) and LiOH·H₂O (10 mg, 238.30 μmol) in THF (1 mL) and H₂O (1 mL) was stirred at 20° C. for 1 h. The mixture was neutralized with FA and then concentrated in vacuo to afford compound 1.665 (50 mg) as a white solid.

LCMS (AM3): rt=0.949 min, (667.3 [M+H]⁺), 85.7% purity.

### Synthesis of Intermediate 1.700

### (S)-5-(3-(3-((tert-butoxycarbonyl)amino)propoxy)pyrrolidin-1-yl)benzo[c][2,6]naphthyridine-8-carboxylic acid 1.698

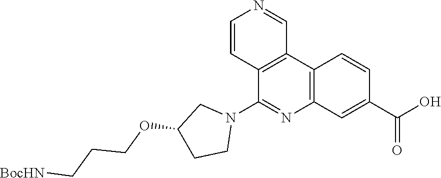 [SMILES: CC(C)(C)OC(=O)NCCCO[C@H]1CCN(c2nc3cc(C(=O)O)ccc3c3cnccc23)C1]

To a solution of compound 1.583 (250 mg, 520.23 μmol) in THF (5 mL), MeOH (5 mL) and H₂O (5 mL) was added LiOH·H₂O (43.66 mg, 1.04 mmol) at 20° C. The reaction mixture was stirred at 20° C. for 2 h. The organic solvents were removed in vacuo and then acidified with aq. HCl solution (1 M) until pH=5. The mixture was extracted with EA (20 mL×2) and the combined organic layer was washed (brine, 30 mL), dried (Na₂SO₄), filtered and concentrated in vacuo to afford compound 1.698 (240 mg, 514.44 μmol, 98.9% yield) as a yellow oil, that was used directly.

### (S)-tert-butyl (3-((1-(8-carbamoylbenzo[c][2,6]naphthyridin-5-yl)pyrrolidin-3-yl)oxy)propyl)carbamate 1.699

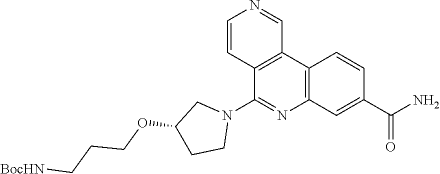 [SMILES: CC(C)(C)OC(=O)NCCCO[C@H]1CCN(c2nc3cc(C(N)=O)ccc3c3cnccc23)C1]

To a solution of compound 1.698 (240 mg, 514.44 μmol) in DMF (3 mL) was added DIPEA (132.98 mg, 1.03 mmol), EDCI (147.93 mg, 771.66 μmol) and HOBt (104.27 mg, 771.66 μmol) at 20° C. After stirring at 20° C. for 0.1 h, NH₄Cl (41.28 mg, 771.66 μmol) was added. The reaction mixture was stirred at 20° C. for 1 h. The reaction mixture was diluted with H₂O (20 mL) and then extracted with EA (30 mL×2). The organic layer was washed (brine, 40 mL), dried (Na₂SO₄), filtered and concentrated in vacuo. The crude product was purified (PM46) to afford compound 1.699 (230 mg, 494.05 μmol, 96.0% yield) as a yellow solid.

LCMS (AM3): rt=0.764 min, (466.3 [M+H]⁺), 100% purity.

### (S)-5-(3-(3-aminopropoxy)pyrrolidin-1-yl)benzo[c][2,6]naphthyridine-8-carboxamide 1.700

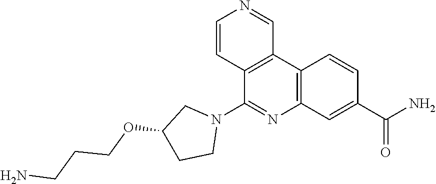 [SMILES: NCCCO[C@H]1CCN(c2nc3cc(C(N)=O)ccc3c3cnccc23)C1]

To a solution of compound 1.699 (230 mg, 494.05 μmol) in DCM (1.5 mL) was added TFA (2.31 g, 20.26 mmol) at 20° C. The reaction mixture was stirred at 20° C. for 1 h. The reaction mixture was concentrated in vacuo to afford compound 1.700 (160 mg, 364.29 μmol, 73.7% yield) as a yellow solid, that was used directly.

LCMS (AM3): rt=0.659 min, (366.2 [M+H]⁺), 68.75% purity.

### Synthesis of Intermediate 1.777

### Methyl 4-oxo-2-(tetrahydro-2H-pyran-2-yl)-4,5-dihydro-2H-pyrazolo[3,4-c]quinoline-7-carboxylate 1.772

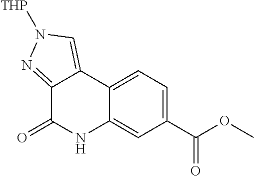

To a mixture of (2-amino-4-(methoxycarbonyl)phenyl) boronic acid (1 g, 4.32 mmol, HCl salt) and methyl 4-iodo-1-(tetrahydro-2H-pyran-2-yl)-1H-pyrazole-3-carboxylate (2.00 g, 5.95 mmol) (Journal of the Chemical Society; Chemical Communications. 1994 (5), 583-584) in 1,4-dioxane (10 mL) was added Cs₂CO₃ (2.82 g, 8.64 mmol) and Pd(dppf)Cl₂ (316.15 mg, 432.08 μmol). The mixture was heated to 120° C. and stirred for 12 h under N₂. The mixture was concentrated in vacuo and the residue was purified (PM21) to afford compound 1.772 (750 mg, 2.29 mmol, 53.0% yield).

LCMS (AM3): rt=0.869 min, (328.5 [M+H]⁺), 76.4% purity.

### Methyl 4-chloro-3H-pyrazolo[3,4-c]quinoline-7-carboxylate 1.773

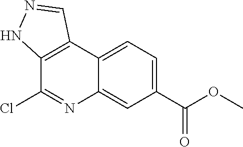 [SMILES: COC(=O)c1ccc2c(c1)nc(Cl)c1[nH]ncc12]

A mixture of compound 1.772 (550 mg, 1.68 mmol) in POCl₃ (11 g, 71.74 mmol) was stirred at 60° C. for 12 h. The mixture was concentrated in vacuo and the residue was diluted with ACN (10 mL). The mixture was added slowly to iced-H₂O (30 mL), then the mixture was adjusted to pH=8 by adding saturated aq. NaHCO₃ solution. The mixture was filtered and the filter cake was dried under vacuum to afford compound 1.773 (500 mg) as a grey solid, which was used directly without further purification.

### Methyl 4-((3-((tert-butoxycarbonyl)amino)propyl)amino)-3H-pyrazolo[3,4-c]quinoline-7-carboxylate 1.774

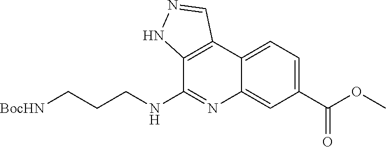 [SMILES: COC(=O)c1ccc2c(c1)nc(NCCCNC(=O)OC(C)(C)C)c1[nH]ncc12]

A mixture of compound 1.773 (600 mg, 2.29 mmol), tert-butyl (3-aminopropyl)carbamate (1.20 g, 6.88 mmol) and DIPEA (592.71 mg, 4.59 mmol) in DMSO (7 mL) was stirred at 70° C. for 12 h The mixture was poured into H₂O (100 mL) and extracted with EA (50 mL×2). The combined organic phase was washed (brine, 50 mL), dried over Na₂SO₄ and concentrated in vacuo. The residue was purified (PM40) to afford compound 1.774 (400 mg) as a brown solid.

LCMS (AM3): rt=0.812 min, (400.4 [M+H]⁺), 68.5% purity.

### Methyl 4-((3-aminopropyl)amino)-3H-pyrazolo[3,4-c]quinoline-7-carboxylate 1.775

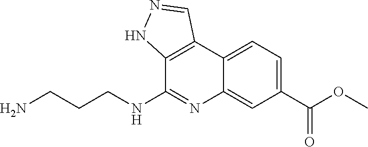 [SMILES: COC(=O)c1ccc2c(c1)nc(NCCCN)c1[nH]ncc12]

Compound 1.774 (380 mg, 951.33 μmol) in a solution of HCl in MeOH (4 mL, 4 M) was stirred at 30° C. for 0.5 h. The mixture was concentrated in vacuo and the residue was purified (PM40) to afford compound 1.775 (160 mg, 476.49 μmol, 50.1% yield, HCl salt) as a brown solid

LCMS (AM3): rt=0.377 min, (300.1 [M+H]⁺), 96.1% purity.

### Methyl 4-((3-(3-((tert-butoxycarbonyl)((2-chloro-[1,1′-biphenyl]-4-yl)methyl)amino)propanamido)propyl)amino)-3H-pyrazolo[3,4-c]quinoline-7-carboxylate 1.776

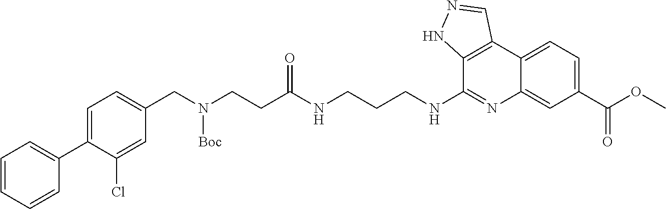 [SMILES: COC(=O)c1ccc2c(c1)nc(NCCCNC(=O)CCN(Cc1ccc(-c3ccccc3)c(Cl)c1)C(=O)OC(C)(C)C)c1[nH]ncc12]

To a mixture of Intermediate D (178.80 mg, 458.62 μmol) and DIPEA (215.54 mg, 1.67 mmol) in DMF (5 mL) was added HATU (190.23 mg, 500.32 μmol) at 30° C. After stirring at 30° C. for 0.5 h, compound 1.775 (140 mg, 416.93 μmol, HCl salt) was added. The mixture was stirred at 30° C. for 3 h. The mixture was poured into H₂O (100 mL) and extracted with EA (50 mL×2). The combined organic phase was washed (brine, 50 mL), dried over Na₂SO₄ and concentrated. The residue was purified (PM95) to afford compound 1.776 (130 mg, 193.69 μmol, 46.5% yield) as a yellow solid.

LCMS (AM3): rt=0.909 min, (671.3 [M+H]⁺), 99.6% purity.

### 4-((3-(3-((Tert-butoxycarbonyl)((2-chloro-[1,1′-biphenyl]-4-yl)methyl)amino)propanamido)propyl)amino)-3H-pyrazolo[3,4-c]quinoline-7-carboxylic acid 1.777

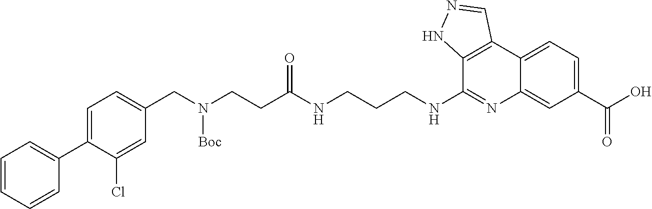 [SMILES: CC(C)(C)OC(=O)N(CCC(=O)NCCCNc1nc2cc(C(=O)O)ccc2c2cn[nH]c12)Cc1ccc(-c2ccccc2)c(Cl)c1]

To a mixture of compound 1.776 (130 mg, 193.69 μmol) in THF (3 mL), MeOH (1 mL) and H₂O (1 mL) was added LiOH·H₂O (32.51 mg, 774.75 μmol) at 30° C. The mixture was stirred at 30° C. for 16 h. The mixture was adjusted to pH=6 by adding TFA and concentrated in vacuo to afford compound 1.777 (160 mg) as a yellow oil, which was used directly without purification.

LCMS (AM3): rt=0.906 min, (657.3 [M+H]⁺), 90.2% purity.

### Synthesis of Intermediate 1.807

### Methyl 3-amino-4-(1-((2-(trimethylsilyl)ethoxy)methyl)-1H-imidazol-5-yl)benzoate 1.805

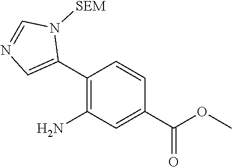 [SMILES: COC(=O)c1ccc(-c2cncn2COCC[Si](C)(C)C)c(N)c1]

A mixture of methyl 3-amino-4-(4,4,5,5-tetramethyl-1,3,2-dioxaborolan-2-yl)benzoate (900 mg, 3.25 mmol) (ACS Medicinal Chemistry Letters, 2020, 11 (5), 713-719), 5-bromo-1-((2-(trimethylsilyl)ethoxy)methyl)-1H-imidazole (ref: WO2017/165139, 0.9 g, 3.25 mmol), Na₂CO₃ (688 mg, 6.49 mmol) and RuPhos Pd G3 (272 mg, 0.32 mmol) in 1,4-dioxane (2 mL) and H₂O (0.2 mL) was stirred under a nitrogen atmosphere at 85° C. for 3 h. The mixture was filtered and concentrated in vacuo to give a residue that was purified (PM4) to afford compound 1.805 (135 mg, 0.39 mmol, 12.0% yield) as a light yellow oil.

LCMS (AM3): rt=0.835 min, (348.2 [M+H]⁺), 38.0% purity.

### Methyl 3-((3-(3-((tert-butoxycarbonyl)((2-chloro-[1,1′-biphenyl]-4-yl)methyl)amino)propanamido)propyl)amino)-4-(1-((2-(trimethylsilyl)ethoxy)methyl)-1H-imidazol-5-yl)benzoate 1.806

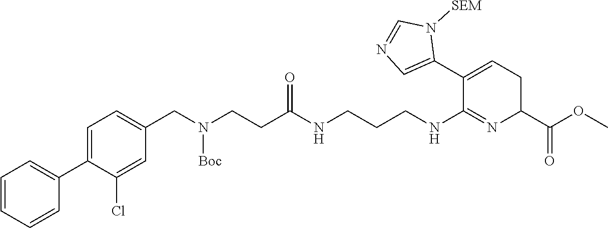 [SMILES: COC(=O)C1CC=C(c2cncn2COCC[Si](C)(C)C)C(NCCCNC(=O)CCN(Cc2ccc(-c3ccccc3)c(Cl)c2)C(=O)OC(C)(C)C)=N1]

A mixture of compound 1.805 (126 mg, 0.36 mmol) and Intermediate H (160 mg, 0.35 mmol) in MeOH (3 mL) was stirred at RT for 1 h, then NaBH(OAc)₃ (147.85 mg, 0.7 mmol) was added. The mixture was stirred at RT for 20 h. The mixture was concentrated in vacuo and the residue was purified by prep-TLC (PM2) to afford compound 1.806 (0.12 g, 0.15 mmol, 42.5% yield) as a colourless oil.

LCMS (AM3): rt=1.089 min, (776.3 [M+H]⁺), 39.8% purity.

### 3-((3-(3-((Tert-butoxycarbonyl)((2-chloro-[1,1′-biphenyl]-4-yl)methyl)amino)propanamido)propyl)amino)-4-(1-((2-(trimethylsilyl)ethoxy)methyl)-1H-imidazol-5-yl)benzoic acid 1.807

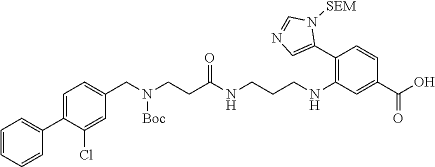 [SMILES: CC(C)(C)OC(=O)N(CCC(=O)NCCCNc1cc(C(=O)O)ccc1-c1cncn1COCC[Si](C)(C)C)Cc1ccc(-c2ccccc2)c(Cl)c1]

To a solution of compound 1.806 (120 mg, 0.15 mmol) in THF (5 mL), H₂O (5 mL) and MeOH (1 mL) was added LiOH·H₂O (12.97 mg, 0.31 mmol) at RT. The mixture was stirred at RT for 18 h. The reaction mixture was diluted with aq. HCl (0.1 M, 30 mL) and extracted with EA (20 mL×2). The combined organic phase was washed (brine, 50 mL), dried (Na₂SO₄), filtered and concentrated in vacuo to afford compound 1.807 (95 mg, 0.12 mmol, 76.6% yield) as a light yellow solid, which was used directly without further purification.

LCMS (AM3): rt=1.045 min, (762.2 [M+H]⁺), 88.9% purity.

### Synthesis of Intermediate 1.813

### 4-Bromo-1-((2-(trimethylsilyl)ethoxy)methyl)-1H-pyrazole-3-carboxamide 1.809

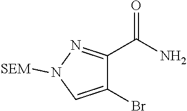 [SMILES: C[Si](C)(C)CCOCn1cc(Br)c(C(N)=O)n1]

To a solution of 4-bromo-1H-pyrazole-3-carboxamide (1 g, 5.26 mmol) in DMF (10 mL) was added NaH (421.06 mg, 10.53 mmol, 60% dispersion in oil) at 0° C. After stirring for 0.5 h, SEMCl (1.05 g, 6.32 mmol) was added. The reaction mixture was warmed to 25° C. and stirred for 1.5 h. The reaction mixture was poured into H₂O (10 mL) and extracted with EA (10 mL×2). The combined organic layer was washed (brine, 10 mL), dried (Na₂SO₄), filtered and concentrated in vacuo. The residue was purified (PM6) to afford compound 1.809 (500 mg, 1.56 mmol, 29.7% yield) as a colourless oil.

¹H NMR (400 MHz, CHCl₃-d) δ: 7.66 (s, 1H), 6.74 (br s, 1H), 5.49 (br s, 1H), 5.40 (s, 2H), 3.60-3.56 (t, 2H), 0.97-0.91 (t, 2H), 0.00 (s, 9H) ppm.

### Methyl 3-amino-4-(3-carbamoyl-1-((2-(trimethylsilyl)ethoxy)methyl)-1H-pyrazol-4-yl)benzoate 1.810

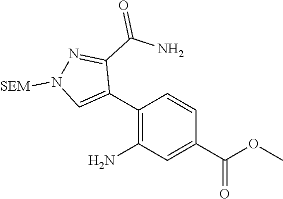 [SMILES: COC(=O)c1ccc(-c2cn(COCC[Si](C)(C)C)nc2C(N)=O)c(N)c1]

A mixture of compound 1.809 (400 mg, 1.25 mmol), Methyl 3-amino-4-(4,4,5,5-tetramethyl-1,3,2-dioxaborolan-2-yl)benzoate (CAS 850689-26-8, 380.74 mg, 1.37 mmol), Pd(PPh₃)₄ (72.16 mg, 62.45 μmol) and Na₂CO₃ (265.12 mg, 2.50 mmol) in DME (1 mL) and H₂O (0.1 mL) was stirred at 90° C. for 12 h under N₂. The mixture was concentrated in vacuo and the residue was purified (PM2) to afford compound 1.810 (240 mg, 460.94 μmol, 36.9% yield) as a red solid

LCMS (AM3): rt=0.885 min, (391.1 [M+H]⁺), 74.5% purity.

### Methyl 3-((3-(3-((tert-butoxycarbonyl)((2-chloro-[1,1′-biphenyl]-4-yl)methyl)amino)propanamido)propyl)amino)-4-(3-carbamoyl-1-((2-(trimethylsilyl)ethoxy)methyl)-1H-pyrazol-4-yl)benzoate 1.811

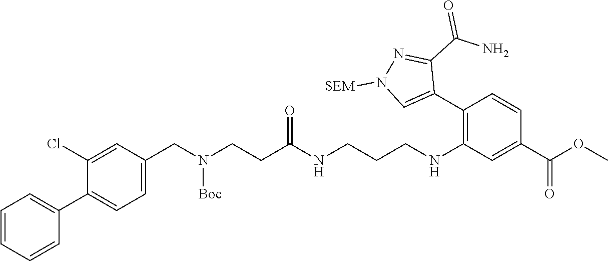 [SMILES: COC(=O)c1ccc(-c2cn(COCC[Si](C)(C)C)nc2C(N)=O)c(NCCCNC(=O)CCN(Cc2ccc(-c3ccccc3)c(Cl)c2)C(=O)OC(C)(C)C)c1]

To a solution of compound 1.810 (230 mg, 516.91 μmol) in MeOH (10 mL) was added Intermediate H (242.23 mg, 620.29 μmol) at 25° C. After stirring for 2 h, NaBH₃CN (64.97 mg, 1.03 mmol) was added. The mixture was stirred at 25° C. for 12 h. The mixture was concentrated in vacuo and the residue was purified (PM46) to afford compound 1.811 (160 mg, 195.25 μmol, 37.8% yield) as a colourless oil.

LCMS (AM3): rt=1.171 min, (819.3 [M+H]⁺), 24.8% purity.

### 3-((3-(3-((Tert-butoxycarbonyl)((2-chloro-[1,1′-biphenyl]-4-yl)methyl)amino)propanamido)propyl)amino)-4-(3-carbamoyl-1-((2-(trimethylsilyl)ethoxy)methyl)-1H-pyrazol-4-yl)benzoic acid 1.812

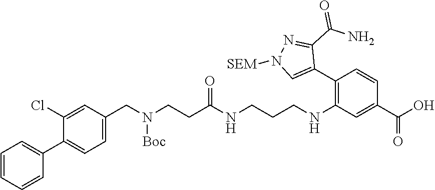 [SMILES: CC(C)(C)OC(=O)N(CCC(=O)NCCCNc1cc(C(=O)O)ccc1-c1cn(COCC[Si](C)(C)C)nc1C(N)=O)Cc1ccc(-c2ccccc2)c(Cl)c1]

To a solution of compound 1.811 (140 mg, 170.84 μmol) in THF (9 mL), H₂O (1 mL) and MeOH (1 mL) was added LiOH·H₂O (71.69 mg, 1.71 mmol) at 25° C. The mixture was stirred at 25° C. for 6 h. The mixture was acidified with aq. HCl (1 M) to pH=4 and extracted with EA (10 mL×2). The combined organic layer was washed (brine, 50 mL), dried (Na₂SO₄), filtered and concentrated in vacuo. The residue was purified (PM96) to afford compound 1.812 (70 mg, 86.13 μmol, 50.4% yield) as a white solid.

LCMS (AM3): rt=1.063 min, (805.4 [M+H]⁺), 99.1% purity.

### 3-((3-(3-((Tert-butoxycarbonyl)((2-chloro-[1,1′-biphenyl]-4-yl)methyl)amino)propanamido)propyl)amino)-4-(3-carbamoyl-1H-pyrazol-4-yl)benzoic acid 1.813

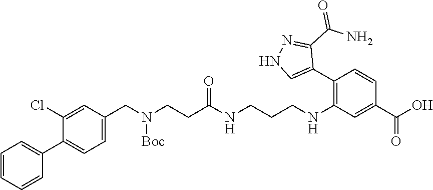 [SMILES: CC(C)(C)OC(=O)N(CCC(=O)NCCCNc1cc(C(=O)O)ccc1-c1c[nH]nc1C(N)=O)Cc1ccc(-c2ccccc2)c(Cl)c1]

To a solution of compound 1.812 (50 mg, 61.52 μmol) in THF (1 mL) was added TBAF·3H₂O (194.1 mg, 615.20 μmol). The mixture was heated to 70° C. and stirred for 12 h. The mixture was concentrated in vacuo and the residue was purified (PM46) to afford compound 1.813 (40 mg, 57.41 μmol, 93.3% yield) as a white solid.

LCMS (AM3): rt=0.922 min, (675.2 [M+H]⁺), 96.9% purity.

### Synthesis of Intermediate 1.816

### Methyl 5-(2-(4-((tert-butoxycarbonyl)amino)butoxy)ethyl)-4-oxo-2-(tetrahydro-2H-pyran-2-yl)-4,5-dihydro-2H-pyrazolo[3,4-c]quinoline-7-carboxylate1.814

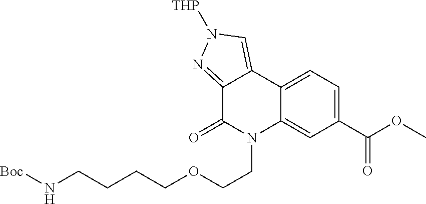

A solution of compound 1.772 (300 mg, 0.92 mmol), Cs₂CO₃ (1.49 g, 1.83 mmol) and compound 1.372 (314 mg, 0.92 mmol) in DMF (2 mL) was stirred at 80° C. for 2 h. The reaction mixture was concentrated in vacuo and the residue was purified (PM97) to afford compound 1.814 (250 mg, 0.46 mmol, 50.3% yield) as a yellow solid.

LCMS (AM3): rt=0.975 min, (543.4 [M+H]⁺), 99.5% purity.

### Methyl 5-(2-(4-aminobutoxy)ethyl)-4-oxo-4,5-dihydro-3H-pyrazolo[3,4-c]quinoline-7-carboxylate 1.815

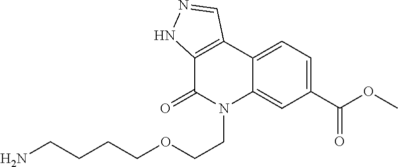 [SMILES: COC(=O)c1ccc2c3cn[nH]c3c(=O)n(CCOCCCCN)c2c1]

To a solution of compound 1.814 (250 mg, 0.46 mmol) in 1,4-dioxane (1 mL) was added a solution of HCl in 1,4-dioxane (2 mL, 4 M) at RT. The mixture was stirred at RT for 0.5 h. The reaction mixture was concentrated in vacuo to afford compound 1.815 (120 mg, HCl salt) as a yellow solid, which was used directly without further purification.

LCMS (AM3): rt=0.645 min, (359.2 [M+H]⁺), 51.4% purity.

### Methyl 5-(2-(4-((3-chloro-4-(trifluoromethoxy)benzyl)amino)butoxy)ethyl)-4-oxo-4,5-dihydro-3H-pyrazolo[3,4-c]quinoline-7-carboxylate 1.816

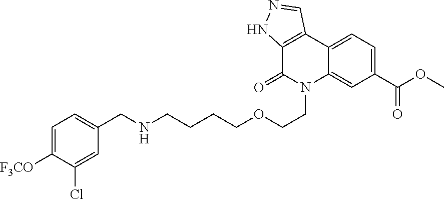 [SMILES: COC(=O)c1ccc2c3cn[nH]c3c(=O)n(CCOCCCCNCc3ccc(OC(F)(F)F)c(Cl)c3)c2c1]

To a mixture of compound 1.815 (73 mg, 0.2 mmol, HCl salt), DIPEA (0.1 mL, 0.06 mmol) and 3-chloro-4-(trifluoromethoxy)benzaldehyde (CAS 83279-39-4, 46 mg, 0.2 mmol) in MeOH (1 mL) was added NaBH₃CN (19 mg, 0.3 mmol) at RT. The mixture was stirred at RT for 16 h. The reaction mixture was concentrated in vacuo and the residue was purified (PM98) to afford compound 1.816 (60 mg, 0.11 mmol, 52.2% yield) as a white solid.

LCMS (AM3): rt=0.862 min, (567.3 [M+H]⁺), 52.2% purity.

### Synthesis of Intermediate 1.820

### Methyl 3-((3-(3-((tert-butoxycarbonyl)(3-chloro-4-(trifluoromethoxy)benzyl)amino)propanamido)propyl)amino)-4-(4,4,5,5-tetramethyl-1,3,2-dioxaborolan-2-yl)benzoate 1.817

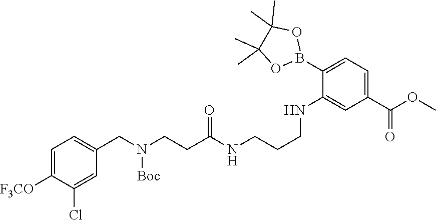 [SMILES: COC(=O)c1ccc(B2OC(C)(C)C(C)(C)O2)c(NCCCNC(=O)CCN(Cc2ccc(OC(F)(F)F)c(Cl)c2)C(=O)OC(C)(C)C)c1]

A mixture of compound 1.325 (400 mg, 0.88 mmol) and compound 1.804 (367 mg, 1.32 mmol) in MeOH (10 mL) was stirred at RT for 2 h, then NaBH₃CN (111 mg, 1.77 mmol) was added. The mixture was stirred at RT for 16 h. The reaction mixture was diluted with H₂O (200 mL) and then extracted with EA (100 mL×3). The combined organic layer was washed (brine, 100 mL×2), dried (Na₂SO₄), filtered and concentrated in vacuo to afford compound 1.817 (600 mg, 0.84 mmol, 95.1% yield) as a yellow oil, which was used directly without further purification.

### Methyl 5-(3-(3-((tert-butoxycarbonyl)(3-chloro-4-(trifluoromethoxy)benzyl)amino)propanamido)propyl)-4-oxo-2-(tetrahydro-2H-pyran-2-yl)-4,5-dihydro-2H-pyrazolo[3,4-c]quinoline-7-carboxylate 1.818

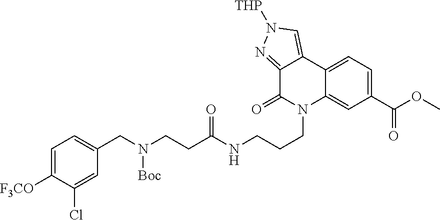

To a solution of compound 1.817 (600 mg, 0.84 mmol) and methyl 4-iodo-1-(tetrahydro-2H-pyran-2-yl)-1H-pyrazole-3-carboxylate (ref: WO2020150545, 282 mg, 0.84 mmol) in 1,4-dioxane (2 mL) was added Cs₂CO₃ (547 mg, 1.68 mmol) and Pd(dppf)Cl₂ (34 mg, 0.04 mmol) at RT. The mixture was refluxed under a nitrogen atmosphere for 14 h. The reaction mixture was diluted with H₂O (20 mL) and the mixture was extracted with EA (30 mL×3). The combined organic layer was washed (brine, 10 mL×2), dried (Na₂SO₄), filtered and concentrated in vacuo. The residue was purified (PM46) to afford compound 1.818 (200 mg, 0.26 mmol, 31.1% yield) as a white solid.

LCMS (AM3): rt=1.080 min, (764.2 [M+H]⁺), 96.9% purity.

### 5-(3-(3-((Tert-butoxycarbonyl)(3-chloro-4-(trifluoromethoxy)benzyl)amino)propanamido)propyl)-4-oxo-2-(tetrahydro-2H-pyran-2-yl)-4,5-dihydro-2H-pyrazolo[3,4-c]quinoline-7-carboxylic acid 1.819

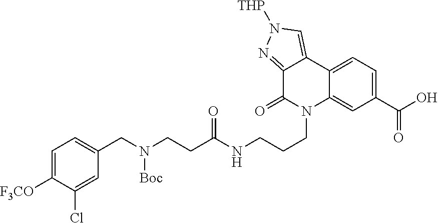

A mixture of compound 1.818 (120 mg, 0.16 mmol) and LiOH·H₂O (19.77 mg, 0.47 mmol) in THF (2 mL) and H₂O (2 mL) was stirred at RT for 2 h. The reaction mixture was neutralized with aq. HCl (1 M) to pH=5 and concentrated in vacuo to afford compound 1.819 (80 mg) as a yellow oil, which was used directly without further purification.

### Tert-butyl (3-((3-(7-carbamoyl-4-oxo-2-(tetrahydro-2H-pyran-2-yl)-2H-pyrazolo[3,4-c]quinolin-5 (4H)-yl)propyl)amino)-3-oxopropyl)(3-chloro-4-(trifluoromethoxy)benzyl)carbamate 1.820

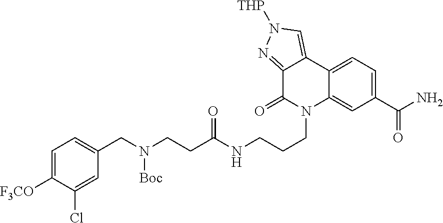

To a mixture of compound 1.819 (70 mg, 0.09 mmol) and NH₄Cl (10 mg, 0.19 mmol) in DMF (1 mL) was added HATU (53 mg, 0.14 mmol) and DIPEA (24 mg, 0.19 mmol) at RT. The mixture was stirred at RT for 1 h. The reaction mixture was diluted with H₂O (20 mL) and extracted with EA (20 mL×3). The combined organic layer was washed (brine, 10 mL×2), dried (Na₂SO₄), filtered and concentrated in vacuo to afford compound 1.820 (70 mg) as a yellow oil, which was used directly without further purification.

LCMS (AM3): rt=1.005 min, (749.3 [M+H]⁺), 67.8% purity.

### Synthesis of Intermediate 1.823

### Methyl 3-((3-(3-((tert-butoxycarbonyl)((2-chloro-[1,1′-biphenyl]-4-yl)methyl)amino)propanamido)propyl)amino)-4-(4,4,5,5-tetramethyl-1,3,2-dioxaborolan-2-yl)benzoate 1.821

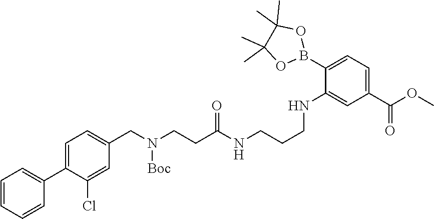 [SMILES: COC(=O)c1ccc(B2OC(C)(C)C(C)(C)O2)c(NCCCNC(=O)CCN(Cc2ccc(-c3ccccc3)c(Cl)c2)C(=O)OC(C)(C)C)c1]

A mixture of Intermediate H (500 mg, 1.12 mmol) and compound 1.804 (467.12 mg, 1.69 mmol) in MeOH (10 mL) was stirred at 25° C. for 2 h, then NaBH₃CN (70.62 mg, 1.12 mmol) was added. The mixture was stirred at 25° C. for additional 16 h. The reaction mixture was diluted with H₂O (200 mL) and extracted with EA (100 mL×3). The combined organic layer was washed (brine, 100 mL×2), dried (Na₂SO₄), filtered and concentrated in vacuo to afford compound 1.821 (750 mg) as a yellow oil, which was used without further purification.

LCMS (AM3): rt=1.187 min, (706.3 [M+H]⁺), 53.5% purity.

### Methyl 6-(3-(3-((tert-butoxycarbonyl)((2-chloro-[1,1′-biphenyl]-4-yl)methyl)amino)propanamido)propyl)-5-oxo-5,6-dihydrobenzo[c][2,6]naphthyridine-8-carboxylate 1.822

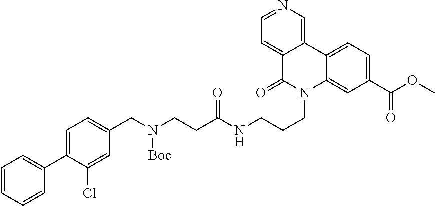 [SMILES: COC(=O)c1ccc2c3cnccc3c(=O)n(CCCNC(=O)CCN(Cc3ccc(-c4ccccc4)c(Cl)c3)C(=O)OC(C)(C)C)c2c1]

A mixture of compound 1.821 (300 mg, 424.88 μmol), methyl 3-bromoisonicotinate (CAS 59786-31-1, 110.15 mg, 509.86 μmol), Cs₂CO₃ (276.87 mg, 849.77 μmol), Pd(dppf)Cl₂ (15.54 mg, 21.24 μmol) in 1,4-dioxane (2 mL) was stirred at 60° C. for 1 h under N₂. The reaction mixture was then heated at 120° C. and stirred for 12 h. The reaction mixture was diluted with H₂O (20 mL) and extracted with EA (50 mL×3). The combined organic layer was washed (brine, 50 mL×2), dried (Na₂SO₄), filtered and concentrated in vacuo. The residue was purified (PM99) to afford compound 1.822 (160 mg, 234.20 μmol, 55.1% yield) as a white solid.

LCMS (AM3): rt=1.071 min, (683.3 [M+H]⁺), 82.1% purity.

### 6-(3-(3-((Tert-butoxycarbonyl)((2-chloro-[1,1′-biphenyl]-4-yl)methyl)amino)propanamido)propyl)-5-oxo-5,6-dihydrobenzo[c][2,6]naphthyridine-8-carboxylic acid 1.823

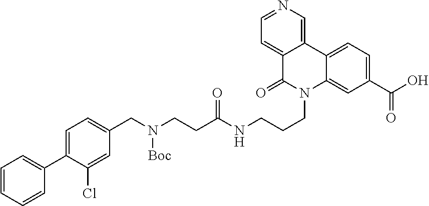 [SMILES: CC(C)(C)OC(=O)N(CCC(=O)NCCCn1c(=O)c2ccncc2c2ccc(C(=O)O)cc21)Cc1ccc(-c2ccccc2)c(Cl)c1]

To a solution of compound 1.822 (120 mg, 175.65 μmol) in H₂O (1 mL) and THF (1 mL) was added LiOH·H₂O (42.07 mg, 1.76 mmol) at 20° C. The mixture was stirred at 20° C. for 0.5 h. The reaction mixture was neutralized with aq. HCl (1 N) to pH=6 and concentrated in vacuo to afford compound 1.823 (100 mg, 149.44 μmol, 85% yield) as a yellow oil, which was used directly without further purification.

LCMS (AM3): rt=0.939 min, (669.3 [M+H]⁺), 74.8% purity.

### Synthesis of Intermediate 1.851

### tert-Butyl(S)-2-((4-((tert-butoxycarbonyl)amino)pentyl)oxy)acetate 1.847

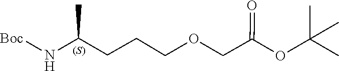

To a solution of tert-butyl N-[(2S)-5-hydroxypentan-2-yl]carbamate (5 g, 24.60 mmol) and tert-butyl 2-bromoacetate (9.90 g, 50.76 mmol) in NaOH aq. (10 g dissolved in 25 mL H₂O) was added TBAI (500 mg, 1.35 mmol) at 0° C. The resulting suspension was stirred at 0° C. for 2 h. The reaction mixture was diluted with H₂O (80 mL) and then extracted with MTBE (80 mL×2). The combined organic phases were washed (brine, 100 mL), dried (Na₂SO₄), filtered and concentrated in vacuo to give the residue. The residue was purified (PM20) to afford compound 1.847 (7 g, 22.05 mmol, 89.6% yield) as colourless oil.

¹H NMR (400 MHz, CHCl₃-d) δ: 4.37 (s, 1H) 3.94 (s, 2H) 3.64 (s, 1H) 3.51 (t, J=6.0 Hz, 2H) 1.70-1.61 (m, 2H) 1.54-1.50 (m, 2H) 1.48 (s, 9H) 1.43 (s, 9H) 1.12 (d, J=6.4 Hz, 3H) ppm.

### tert-Butyl(S)-(5-(2-hydroxyethoxy)pentan-2-yl)carbamate 1.848

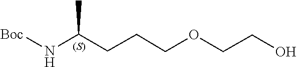

To a mixture of LAH (900.00 mg, 23.71 mmol) in cooled THF (50 mL) at 0° C. was added a solution of compound 1.847 (6 g, 18.90 mmol) in THF (50 mL) slowly. The reaction mixture was stirred at 0° C. for 2 h. The reaction mixture was quenched by addition of H₂O (1 mL), NaOH aq. (15% wt., 1 mL) followed by H₂O (3 mL) slowly at 0° C. After being stirred for 0.5 h, Na₂SO₄ (10 g) was added. The resulting suspension was stirred for 0.5 h and then filtered. The filter cake was washed with EA (100 mL). The filtrate was concentrated in vacuo to give the residue. The residue was purified (PM3) to afford compound 1.848 (3.5 g, 14.15 mmol, 74.8% yield) as colourless oil.

¹H NMR (400 MHz, CHCl₃-d) δ: 3.75-3.72 (m, 2H), 3.70-3.65 (m, 1H), 3.55-3.52 (m, 2H), 3.51-3.48 (m, 2H), 1.69-1.60 (m, 2H), 1.53-1.48 (m, 2H), 1.45 (s, 9H), 1.13 (d, J=6.4 Hz, 3H) ppm.

### Methyl (S)-5-(2-((4-((tert-butoxycarbonyl)amino)pentyl)oxy)ethoxy)benzo[c][2,6]naphthyridine-8-carboxylate 1.849

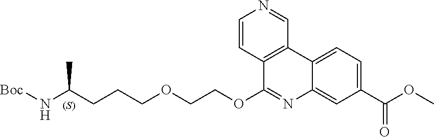

To the mixture of compound 1.848 (500 mg, 2.02 mmol) in THF (10 mL) was added NaH (88.94 mg, 2.22 mmol, 60% wt.) at 0° C. After being stirred at 0° C. for 0.5 h, compound 1.1 (551.26 mg, 2.02 mmol) was added, the reaction mixture was stirred at 0° C. for 2 h. The reaction mixture was quenched by slow addition of H₂O (0.3 mL) at 0° C. and then concentrated in vacuo to afford compound 1.849 (1 g, crude) as yellow solid.

LCMS (AM3): rt=1.007 min, (484.2 [M+H]⁺), 40.2% purity.

### (S)-5-(2-((4-((tert-butoxycarbonyl)amino)pentyl)oxy)ethoxy)benzo[c][2,6]naphthyridine-8-carboxylic acid 1.850

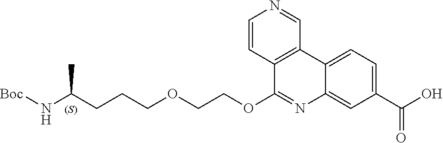

To a solution of compound 1.849 (1 g, 2.07 mmol) in THF (10 mL) and H₂O (5 mL) was added LiOH·H₂O (200 mg, 4.77 mmol) at 20° C. The reaction mixture was stirred at 20° C. for 12 h. The reaction mixture was concentrated in vacuo. The mixture was purified (PM46) to afford compound 1.850 (600 mg, 1.11 mmol, 53.7% yield) as yellow solid.

LCMS (AM3): rt=0.928 min, (470.0 [M+H]⁺), 87.5% purity.

### (S)-5-(2-((4-aminopentyl)oxy)ethoxy)benzo[c][2,6]naphthyridine-8-carboxylic acid 1.851

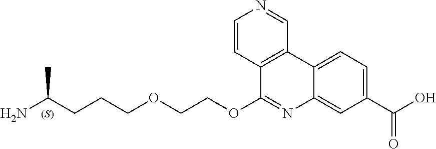

To a mixture of compound 1.850 (600 mg, 1.28 mmol) in DCM (10 mL) was added TFA (145.71 mg, 1.28 mmol) at 20° C. The reaction mixture was stirred at 20° C. for 0.5 h. The reaction mixture was concentrated in vacuo to give the crude product. The crude product was purified (PM46) to afford compound 1.851 (230 mg, 570.04 μmol, 44.6% yield) as white solid.

LCMS (AM3): rt=0.876 min, (369.9 [M+H]⁺), 91.9% purity.

### Synthesis of Intermediate 1.858

### tert-Butyl 2-((3-((tert-butoxycarbonyl)amino)cyclobutyl)methoxy)acetate 1.855

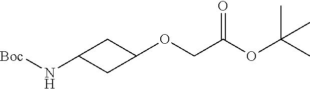 [SMILES: CC(C)(C)OC(=O)COC1CC(NC(=O)OC(C)(C)C)C1]

To a solution of tert-butyl (3-(hydroxymethyl)cyclobutyl)carbamate (CAS 130369-05-0, 5 g, 24.84 mmol), tert-butyl 2-bromoacetate (9.69 g, 49.69 mmol) in NaOH aq. (10 g dissolved in 25 mL H₂O) was added TBAI (458.81 mg, 1.24 mmol) at 0° C. The reaction mixture was stirred at 0° C. for 2 h. The reaction mixture was diluted with H₂O (100 mL) and then extracted with MTBE (100 mL×2). The combined organic phases were washed (brine, 150 mL), dried (Na₂SO₄), filtered and concentrated in vacuo to give the residue. The residue was purified (PM20) to afford compound 1.855 (9.8 g, crude) as colourless oil.

¹H NMR (400 MHz, CHCl₃-d) δ: 4.88-4.63 (m, 1H), 4.25-4.15 (m, 0.5H), 4.08-3.96 (m, 0.5H), 3.94 (d, J=6.0 Hz, 2H), 3.54 (d, J=7.2 Hz, 1H), 3.45 (d, J=5.6 Hz, 1H), 2.48-2.43 (m, 1H), 2.27-2.19 (m, 1.5H), 2.02-1.96 (m, 1H), 1.76-1.54 (m, 1.5H), 1.48 (s, 9H) 1.42 (s, 9H) ppm.

### tert-Butyl (3-((2-hydroxyethoxy)methyl)cyclobutyl)carbamate 1.856

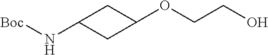 [SMILES: CC(C)(C)OC(=O)NC1CC(OCCO)C1]

To a solution of compound 1.855 (3 g, 9.51 mmol) in cooled THF (40 mL) at 0° C. was added LAH (400 mg, 10.54 mmol) slowly. The reaction mixture was stirred at 0° C. for 2 h. The reaction mixture was quenched by addition of H₂O (0.4 mL) slowly followed by 15% wt. aq. NaOH (0.4 mL) solution and H₂O (1.2 mL) at 0° C. After being stirred for 0.5 h, Na₂SO₄ (5 g) was added, the resulting suspension was stirred for 0.5 h and then filtered and concentrated in vacuo to give a residue which was purified (PM3) to afford compound 1.856 (1 g, 4.08 mmol, 42.8% yield) as colourless oil.

¹H NMR (400 MHz, CHCl₃-d) δ: 4.74 (s, 1H), 4.28-4.15 (m, 0.5H), 4.05-3.95 (m, 0.5H), 3.75-3.72 (m, 2H), 3.57-3.54 (m, 2H), 3.53-3.51 (m, 1H), 3.42 (d, J=5.6 Hz, 1H), 2.49-2.41 (m, 1.5H), 2.25-2.16 (m, 1.5H), 2.02-1.95 (m, 1H), 1.66-1.58 (m, 1H), 1.44 (s, 9H) ppm.

### 5-(2-((3-((tert-Butoxycarbonyl)amino)cyclobutyl)methoxy)ethoxy)benzo[c][2,6]naphthyridine-8-carboxylic acid 1.857

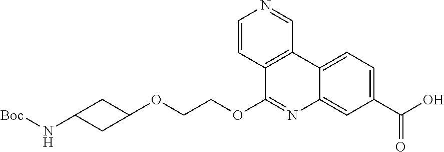 [SMILES: CC(C)(C)OC(=O)NC1CC(OCCOc2nc3cc(C(=O)O)ccc3c3cnccc23)C1]

To a solution of compound 1.856 (1 g, 4.08 mmol) in THF (20 mL) was added NaH (200 mg, 5.00 mmol, 60% wt.) slowly at 0° C. After being stirred for 0.5 h at 0° C., compound 1.1 (1.11 g, 4.08 mmol) was added. The reaction mixture was warmed to 20° C. and stirred for 1.5 h. After completion of the reaction, a solution of LiOH·H₂O (342.12 mg, 8.15 mmol) in H₂O (4 mL) was added. The reaction mixture was stirred at 20° C. for 14 h. The reaction mixture was concentrated in vacuo and the residue purified (PM46) to afford compound 1.857 (1 g, 2.05 mmol, 50.3% yield) as light yellow solid.

LCMS (AM3): rt=0.914 min, (468.0 [M+H]⁺), 94.9% purity.

### 5-(2-((3-Aminocyclobutyl)methoxy)ethoxy)benzo[c][2,6]naphthyridine-8-carboxylic acid 1.858

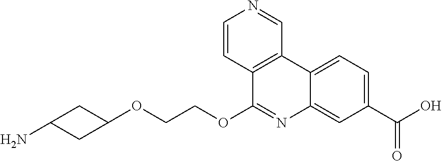 [SMILES: NC1CC(OCCOc2nc3cc(C(=O)O)ccc3c3cnccc23)C1]

To a solution of compound 1.857 (1 g, 2.14 mmol) in DCM (10 mL) was added TFA (243.89 mg, 2.14 mmol) at 20° C. The reaction mixture was stirred at 20° C. for 2 h. The reaction mixture was concentrated in vacuo to give the residue. The residue was purified (PM46) to afford compound 1.858 (320 mg, 741.18 μmol, 34.65% yield, FA salt) as white solid.

LCMS (AM5): rt=0.873 min, (368.2 [M+H]⁺), 95.7% purity.

### Synthesis of Intermediate 1.882

### Methyl 1-(4-((tetrahydro-2H-pyran-2-yl)oxy)butyl)-1H-1,2,3-triazole-4-carboxylate 1.878

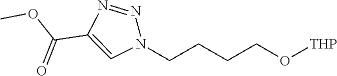

To a solution of 2-(4-azidobutoxy)tetrahydro-2H-pyran (CAS 1391031-38-1, 14 g, 70.26 mmol) and CuI (2.68 g, 14.05 mmol) in a mixed solution of DMF (100 mL) and MeOH (15 mL) was added methyl prop-2-ynoate (CAS 922-67-8, 7.07 g, 84.09 mmol). The reaction mixture was heated to 100° C. and stirred for 2 h. The reaction mixture was filtered and the filtrate was diluted with H₂O (400 mL), the resulting mixture was extracted with EA (100 ml×3). The combined organic phase was washed (brine, 300 mL), dried (Na₂SO₄), filtered and concentrated in vacuum. The residue was purified (PM4) to afford compound 1.878 (6.4 g, crude) as brown oil

LCMS (AM3): rt=0.805 min, (200.1 [M-THP+H]⁺), 54.3% purity.

### 1-(4-((Tetrahydro-2H-pyran-2-yl)oxy)butyl)-1H-1,2,3-triazole-4-carboxamide 1.879

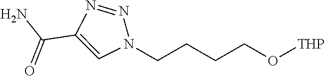

A mixture of compound 1.878 (6.4 g, 22.59 mmol) in NH₃/MeOH (60 mL, saturated with ammonia, 7 M) was stirred at RT for 16 h. The reaction mixture was concentrated in vacuum. The residue was triturated in MTBE (100 mL) and filtered. The filter cake was dried in vacuo to afford compound 1.879 (4.6 g, 15.43 mmol, 68.3% yield) as white solid.

LCMS (AM3): rt=0.617 min, (291.1 [M+Na]⁺), 94.2% purity.

### 1-(4-((Tetrahydro-2H-pyran-2-yl)oxy)butyl)-1H-1,2,3-triazol-4-amine 1.880

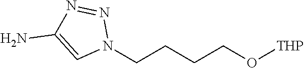

To a solution of NaOH (4.11 g, 102.87 mmol) in H₂O (50 mL) was added Br₂ (4.11 g, 25.69 mmol) at 0° C. After being stirred at 0° C. for 30 min, compound 1.879 (4.6 g, 17.14 mmol) was added. The reaction mixture was warmed to RT and stirred for 30 min, then the mixture was heated to 80° C. and stirred for 2 h. The reaction solution was purified (PM46) and the eluent was lyophilized to afford compound 1.880 (1.5 g, crude) as brown oil.

LCMS (AM3): rt=0.443 min, (157.2 [M−THP+H]⁺), 36.8% purity.

### N-(3,5-difluoro-4-(trifluoromethoxy)benzyl)-1-(4-((tetrahydro-2H-pyran-2-yl)oxy)butyl)-1H-1,2,3-triazol-4-amine 1.881

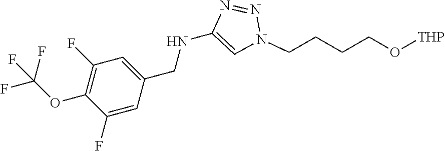

A mixture of compound 1.880 (1.60 g, 7.09 mmol), 3,5-Difluoro-4-(trifluoromethoxy)benzaldehyde (ref: WO2022185041, 1.2 g, 4.99 mmol) and AcOH (597.97 mg, 9.96 mmol) in THF (20 mL) was stirred at 80° C. for 0.5 h. After then the mixture was cooled to 50° C., NaBH(OAc)₃ (3.18 g, 14.99 mmol) was added. The reaction mixture was stirred at 50° C. for 0.5 h. The reaction mixture was concentrated in vacuum. The residue was purified (PM46) and the eluent was lyophilized to afford compound 1.881 (90 mg, 171.85 μmol, 3.4% yield) as brown oil.

LCMS (AM3): rt=0.820 min, (451.2 [M+H]+), 86.4% purity.

### 4-(4-((3,5-Difluoro-4-(trifluoromethoxy)benzyl)amino)-1H-1,2,3-triazol-1-yl) butan-1-ol 1.882

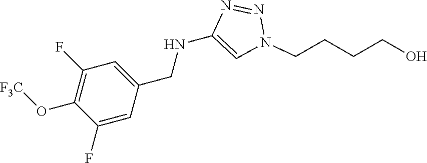 [SMILES: OCCCCn1cc(NCc2cc(F)c(OC(F)(F)F)c(F)c2)nn1]

A mixture of compound 1.881 (90 mg, 199.82 μmol) and TFA (27.01 mmol, 2.00 mL) in DCM (2 mL) was stirred at RT for 4 h. The residue was dissolved in MeOH (4 mL) and then K₂CO₃ (149.48 mg, 1.08 mmol) was added. The mixture was stirred at RT for 2 h and then concentrated in vacuum. The residue was purified (PM46) to afford compound 1.882 (30 mg, 80.27 μmol, 37.1% yield) as brown oil.

LCMS (AM3): rt=0.883 min, (367.1 [M+H]⁺), 98.3% purity.

### Synthesis of Intermediate 1.887

### tert-Butyl (2-(1-(2-hydroxyethyl)-1H-1,2,3-triazol-4-yl)ethyl)carbamate 1.884

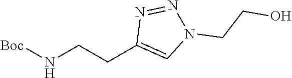 [SMILES: CC(C)(C)OC(=O)NCCc1cn(CCO)nn1]

To a solution of 2-azidoethan-1-ol (CAS 1517 May 1, 910.44 mg, 10.46 mmol) and tert-butyl but-3-yn-1-ylcarbamate (CAS 149990-27-2, 2.3 g, 13.59 mmol) in a mixed solution of DMF (8 mL) and MeOH (2.4 mL) was added CuI (398.24 mg, 2.09 mmol). The mixture was heated to 100° C. and stirred for 2 h under N₂. The reaction mixture was diluted with H₂O (50 mL) and extracted with EA (50 mL×3). The combined organic layers were washed (brine, 30 mL×3), dried (Na₂SO₄), filtered and concentrated in vacuo to give a residue. The mixture was purified (PM46) to afford compound 1.884 (1.8 g, 4.92 mmol, 47.0% yield) as blue oil.

LCMS (AM3): rt=0.776 min, (257.2 [M+H]⁺), 69.7% purity.

### Methyl 5-(2-(4-(2-((tert-butoxycarbonyl)amino)ethyl)-1H-1,2,3-triazol-1-yl)ethoxy)benzo[c][2,6]naphthyridine-8-carboxylate 1.885

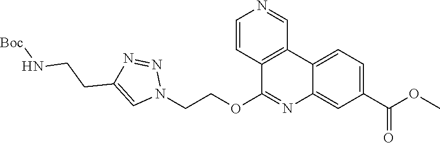 [SMILES: COC(=O)c1ccc2c(c1)nc(OCCn1cc(CCNC(=O)OC(C)(C)C)nn1)c1ccncc12]

To a solution of compound 1.884 (500 mg, 1.95 mmol) in THF was added NaH (93.63 mg, 2.34 mmol, 60% purity) at 0° C. After being stirred for 0.5 h, compound 1.1 (531.97 mg, 1.95 mmol) was added, the resulting mixture was warmed to 25° C. and stirred for 1.5 h. The reaction mixture was quenched by addition of H₂O (10 mL) and extracted with EA (30 mL×3). The combined organic layers were washed (brine, 30 mL×2), dried (Na₂SO₄), filtered and concentrated in vacuo to afford compound 1.885 (700 mg, crude) as brown solid.

LCMS (AM3): rt=0.767 min, (393.2 [M−Boc+H]⁺), 34.9% purity.

### 5-(2-(4-(2-((tert-Butoxycarbonyl)amino)ethyl)-1H-1,2,3-triazol-1-yl)ethoxy)benzo[c][2,6]naphthyridine-8-carboxylic acid 1.886

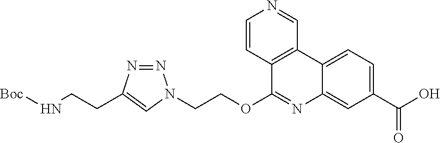 [SMILES: CC(C)(C)OC(=O)NCCc1cn(CCOc2nc3cc(C(=O)O)ccc3c3cnccc23)nn1]

To a solution of compound 1.885 (500 mg, 1.02 mmol) in THF (5 mL) and H₂O (1 mL) was added LiOH·H₂O (127.79 mg, 3.05 mmol) at 25° C. The mixture was stirred at 25° C. for 0.5 h. The mixture was concentrated in vacuum to give a residue. The residue was purified (PM46) and the eluent was lyophilized to afford compound 1.886 (180 mg, 376.18 μmol, 37.06% yield) as white solid.

LCMS (AM3): rt=0.747 min, (479.2 [M+H]⁺), 32.7% purity

### 5-(2-(4-(2-Aminoethyl)-1H-1,2,3-triazol-1-yl)ethoxy)benzo[c][2,6]naphthyridine-8-carboxylic acid 1.887

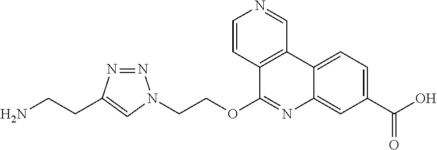 [SMILES: NCCc1cn(CCOc2nc3cc(C(=O)O)ccc3c3cnccc23)nn1]

To a solution of compound 1.886 (180 mg, 376.18 μmol) in DCM (5 mL) was added TFA (1.54 g, 13.51 mmol) at 25° C. The mixture was stirred at 25° C. for 1 h. The mixture was concentrated in vacuum to give a residue. The residue was purified (PM46) and the eluent was lyophilized to afford compound 1.887 (150 mg, 353.43 μmol, 93.95% yield, FA salt) as yellow oil,

LCMS (AM3): rt=0.708 min, (379.2 [M+H]⁺), 100% purity.

### EXAMPLE COMPOUNDS

The Examples are prepared according to the methods below using the Preparations hereinbefore. Wherein additional materials have been prepared, preparations are included for each Example. Alternatively, wherein commercially available materials are used, only the final steps are included, and no intermediate reference number is necessary.

### Example 1

### 5-[4-({4-[({2-chloro-[1,1′-biphenyl]-4-yl}methyl)amino]butyl}amino)butyl]benzo[c]2,6-naphthyridine-8-carboxylic acid

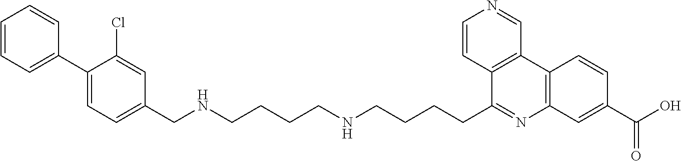 [SMILES: O=C(O)c1ccc2c(c1)nc(CCCCNCCCCNCc1ccc(-c3ccccc3)c(Cl)c1)c1ccncc12]

To a solution of compound 1.83 (20 mg, 34.4 μmol) in MeOH (2 mL) and H₂O (0.5 mL) was added NaOH (11 mg, 275 μmol) and the mixture was stirred at 25° C. for 1 hr. The reaction mixture was diluted with H₂O (4 mL) and concentrated in vacuo to remove MeOH. The residue was acidified with aq. HCl (1 M), then concentrated in vacuo and purified (PM100) to afford EXAMPLE 1 (2.93 mg, 4.74 μmol, 13.8% yield, HCl salt) as a white solid.

LCMS (AM3): RT=0.792 min, (567.5 [M+H]⁺), 97.6% purity.

¹H NMR (400 MHz, MeOH-d₄) δ: 10.58 (s, 1H), 9.17-8.96 (m, 3H), 8.89 (s, 1H), 8.52-8.48 (d, 1H), 7.74 (s, 1H), 7.56 (d, J=7.8 Hz, 1H), 7.51-7.37 (m, 6H), 4.28 (s, 2H), 3.73-3.63 (m, 2H), 3.25-3.09 (m, 6H), 2.28-2.15 (m, 2H), 2.02 (d, J=9.6 Hz, 2H), 1.87 (br s, 4H) ppm.

### Example 2

### 5-{[2-({4-[({2-chloro-[1,1′-biphenyl]-4-yl}methyl)amino]butyl}amino)ethyl]amino}benzo[c]2,6-naphthyridine-8-carboxylic acid

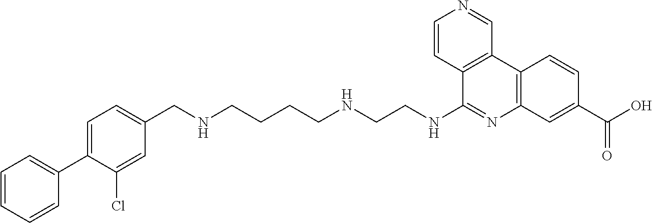 [SMILES: O=C(O)c1ccc2c(c1)nc(NCCNCCCCNCc1ccc(-c3ccccc3)c(Cl)c1)c1ccncc12]

A solution of compound 1.85 (200 mg, 306 μmol) in EA (20 mL) was treated with a solution of HCl in EA (4 M, 20 mL) and stirred at 25° C. for 2 h under N₂. The reaction mixture was concentrated in vacuo and purified (PM101) The resulting residue was triturated with H₂O (100 mL) to give EXAMPLE 2 (8.59 mg, 15.1 μmol, 5% yield) as a yellow solid.

LCMS (AM3): rt=0.760 min, (554.3 [M+H]⁺), 98.7% purity.

¹H NMR (400 MHz, MeOH-d₄) δ: 10.27 (s,EA 1H), 9.08 (d, J=6.0 Hz, 1H), 8.85 (d, J=6.1 Hz, 1H), 8.80 (d, J=8.5 Hz, 1H), 8.70 (s, 1H), 8.20-8.17 (m, 1H), 7.72 (d, J=1.6 Hz, 1H), 7.56-7.52 (m, 1H), 7.49-7.37 (m, 6H), 4.31-4.21 (m, 4H), 3.59 (t, J=5.7 Hz, 2H), 3.17 (s, 4H), 1.96-1.88 (s, 4H) ppm.

### Example 3

### 5-{[3-({4-[({2-chloro-[1,1′-biphenyl]-4-yl}methyl)amino]butyl}amino)propyl]amino}benzo[c]2,6-naphthyridine-8-carboxylic acid

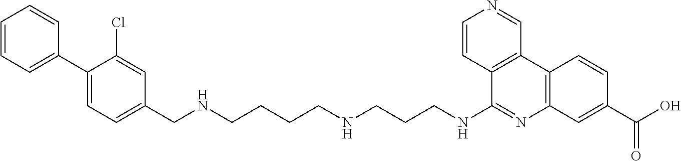 [SMILES: O=C(O)c1ccc2c(c1)nc(NCCCNCCCCNCc1ccc(-c3ccccc3)c(Cl)c1)c1ccncc12]

To a solution of compound 1.87 (150 mg, 224 μmol) in EA (10 mL) was added a solution of HCl in EA (4 M, 20 mL) and the mixture stirred at 25° C. for 2 h. The reaction mixture was concentrated in vacuo and purified (PM101) to give EXAMPLE 3 (11.07 mg, 19.1 μmol, 8.5% yield) as a yellow solid.

LCMS (AM3): rt=0.760 min, (568.3 [M+H]⁺), 98.9% purity

¹H NMR (400 MHz, MeOH-d₄) δ: 10.04 (s, 1H), 8.89 (d, J=5.6 Hz, 1H), 8.73 (d, J=8.4 Hz, 1H), 8.45 (d, J=1.3 Hz, 1H), 8.24 (d, J=5.6 Hz, 1H), 8.07 (d, J=8.4 Hz, 1H), 7.67 (s, 1H), 7.49-7.38 (m, 7H), 4.25 (s, 2H), 3.93 (t, J=6.4 Hz, 2H), 3.20-3.16 (t, 2H), 3.15-3.10 (t, 2H), 3.09-3.04 (t, 2H), 2.25-2.17 (quin, J=6.8 Hz, 2H), 1.94-1.83 (m, 2H), 1.82-1.72 (m, 2H) ppm.

### Example 4

### 5-((2-((2-(((2-chloro-[1,1′-biphenyl]-4-yl)methyl)amino)ethyl)amino)ethyl)amino)benzo[c][2,6]naphthyridine-8-carboxylic acid

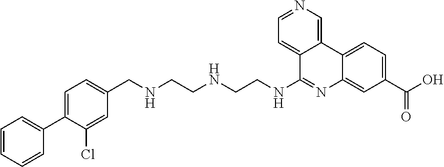 [SMILES: O=C(O)c1ccc2c(c1)nc(NCCNCCNCc1ccc(-c3ccccc3)c(Cl)c1)c1ccncc12]

To a mixture of compound 1.37 (40 mg, 0.063 mmol) in THF (1 mL) and H₂O (1 mL) was added LiOH·H₂O (11 mg, 0.262 mmol) at RT. The reaction mixture was stirred at RT for 8 h, then the reaction mixture was acidified with conc. HCl (1 mL) and stirred at RT for 4 h. The reaction mixture was concentrated in vacuo and purified (PM102) to afford EXAMPLE 4 (17.7 mg, 46.1% yield, HCl salt) as a yellow solid.

LCMS (AM1): rt=0.620 min, (526.1 [M+H]⁺), 96.8% purity

¹H NMR (400 MHz, MeOH-d₄) δ: 10.23 (s, 1H), 9.02 (d, J=6 Hz, 1H), 8.77 (d, J=8.8 Hz, 1H), 8.72 (d, J=6 Hz, 1H), 8.62 (s, 1H), 8.15 (dd, J=8.8 Hz, 1.6 Hz, 1H), 7.73 (d, J=1.6 Hz, 1H), 7.56 (dd, J=8.0 Hz, 1.6 Hz, 1H), 7.47-7.36 (m, 6H), 4.34 (s, 2H), 4.23 (t, J=5.2 Hz, 2H), 3.67 (t, J=5.2 Hz, 2H), 3.62-3.57 (m, 4H) ppm.

### Example 5

### 5-(3-(3-(((2-Chloro-[1,1′-biphenyl]-4-yl)methyl)amino)propanamido)propyl)-4-oxo-4,5-dihydro-3H-pyrazolo[3,4-c]quinoline-7-carboxylic acid

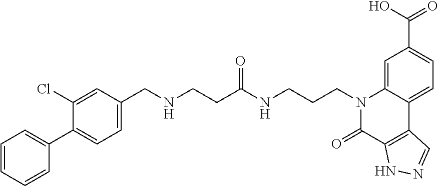 [SMILES: O=C(CCNCc1ccc(-c2ccccc2)c(Cl)c1)NCCCn1c(=O)c2[nH]ncc2c2ccc(C(=O)O)cc21]

To a solution of compound 1.813 (35 mg, 50.23 μmol) in H₂O (1 mL) was added aq. HCl (5 mL, 2 M) at 20° C. The mixture was stirred at 20° C. for 12 h. The reaction mixture was concentrated in vacuo and the residue was purified (PM103) to afford EXAMPLE 5 (9.45 mg, 15.68 μmol, 31.2% yield, HCl salt) as an off-white solid.

LCMS (AM3): rt=0.804 min, (558.1 [M+H]⁺), 98.7% purity.

¹H NMR (400 MHz, MeOH-d₄) δ: 8.54 (d, J=2.0 Hz, 1H), 8.21 (s, 1H), 8.16 (dd, J=8.0, 2.0 Hz, 1H), 7.99 (d, J=8.0 Hz, 1H), 7.71 (d, J=1.2 Hz, 1H), 7.54-7.52 (m, 1H), 7.44-7.34 (m, 6H), 4.36 (s, 2H), 3.42-3.35 (m, 6H), 2.77 (t, J=6.4 Hz, 2H), 2.08-2.01 (m, 2H) ppm.

### Example 6

### 5-((2-(4-(((2-Chloro-[1,1′-biphenyl]-4-yl)methyl)amino)butanamido)ethyl)amino)benzo[c][2,6]naphthyridine-8-carboxylic acid

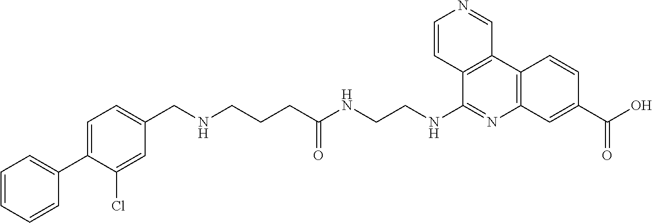 [SMILES: O=C(CCCNCc1ccc(-c2ccccc2)c(Cl)c1)NCCNc1nc2cc(C(=O)O)ccc2c2cnccc12]

A mixture of compound 1.388 (220 mg, 0.322 mmol) in aq. HCl (1 M, 2 mL) was stirred at 80° C. for 15 h, then the reaction mixture was cooled to 0° C. NaOH (129 mg, 3.23 mmol) was added at 0° C. and the reaction mixture was heated to 50° C. and stirred for 5 h. The reaction mixture was concentrated in vacuo and the residue was purified (PM104) to afford EXAMPLE 6 (50 mg, 34.7% yield, TFA salt) as a light yellow solid.

LCMS (AM1): rt=0.642 min, (568.2 [M+H]⁺), 99.1% purity.

¹H NMR (400 MHz, MeOH-d₄) δ: 10.08 (s, 1H), 8.95 (d, J=5.6 Hz, 1H), 8.77 (d, J=8.4 Hz, 1H), 8.65 (s, 1H), 8.25 (d, J=5.6 Hz, 1H), 8.15 (d, J=8.0 Hz, 1H), 7.67 (d, J=1.6 Hz, 1H), 7.49-7.35 (m, 7H), 4.27 (s, 2H), 3.91 (t, J=6.4 Hz, 2H), 3.64 (t, J=6.4 Hz, 2H), 3.24 (t, J=7.2 Hz, 2H), 2.52 (t, J=6.4 Hz, 2H), 2.10-2.02 (quin, 2H) ppm.

### Example 7

### 5-((2-((4-(((2-chloro-[1,1′-biphenyl]-4-yl)methyl)amino)butyl)amino)ethyl)amino)benzo[c][2,6]naphthyridine-8-carboxamide

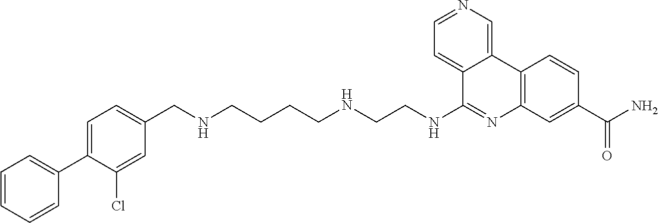 [SMILES: NC(=O)c1ccc2c(c1)nc(NCCNCCCCNCc1ccc(-c3ccccc3)c(Cl)c1)c1ccncc12]

A mixture of compound 1.25 (160 mg, 0.19 mmol) and TFA (5 mL, 67.53 mmol) in DCM (10 mL) was stirred at RT for 2 h. The reaction mixture was concentrated in vacuo to give crude product which was purified (PM44) to afford EXAMPLE 7 (87 mg, 57.5% yield, TFA salt) as a yellow solid.

LCMS (AM4): rt=0.588 min, (553.2 [M+H]⁺), 96.8% purity

¹H NMR (400 MHz, MeOH-d₄) δ: 10.03 (s, 1H), 8.87 (d, J=5.7 Hz, 1H), 8.70 (d, J=8.6 Hz, 1H), 8.28 (d, J=1.7 Hz, 1H), 8.19 (d, J=5.7 Hz, 1H), 7.90 (dd, J=1.8, 8.4 Hz, 1H), 7.68 (d, J=1.6 Hz, 1H), 7.52-7.48 (m, 1H), 7.48-7.37 (m, 6H), 4.26 (s, 2H), 4.10-4.03 (t, 2H), 3.50-3.44 (t, 2H), 3.30-3.25 (m, 2H), 3.18-3.11 (m, 2H), 1.97-1.80 (m, 4H) ppm.

The following Examples in Table 3 were made with non-critical changes or substitutions to the exemplified deprotection procedure in EXAMPLE 7 that would be understood by one skilled in the art, using suitably Boc-protected intermediates as described in the scheme below.

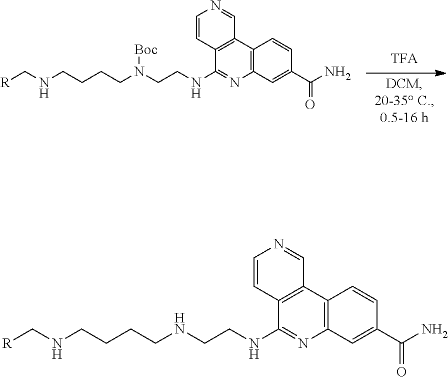

TABLE 3

| | | R | |
|---|---|---|---|
| EXAMPLE | Chemical | (Intermediate | |
| No. | IUPAC name | No.) | Analytical |
| EXAMPLE 8 | 5-((2-((4-((3- Chlorobenzyl) amino)butyl) amino)ethyl) amino)benzo[c] [2,6]naph- thyridine-8- | 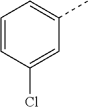 | ¹H NMR (400 MHz, MeOH-d₄) δ: 10.01 (s, 1H), 8.87 (d, J = 5.7 Hz, 1H), 8.65 (d, J = 8.4 Hz, 1H), 8.28 (d, J = 1.7 Hz, 1H), 8.24 (d, J = 5.7 Hz, 1H), 7.89 (dd, J = 1.8, 8.5 Hz, 1H), 7.55 (d, J = 1.3 Hz, 1H), 7.47-7.41 (m, 3H), 4.20 (s, 2H), 4.08 (t, J = 5.4 Hz, 2H), 3.48 (t, J = 5.4 Hz, 2H), |
| | carboxamide | (compound | 3.25 (t, J = 7.2 Hz, 2H), 3.11 (t, J = 7.2 Hz, |
| | | 1.252) | 2H), 1.78-1.92 (m, 4H) ppm. |
| | | | LCMS (AM3): rt = 0.669 min, (477.1 |
| | | | [M + H]⁺), 100% purity. |
| | | | Purification Method PM105 |
| EXAMPLE 12 | 5-((2-((4- (((1H-Indol-2- yl)methyl)amino) butyl)amino) ethyl)amino) benzo[c][2,6] naphthyridine- | 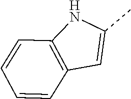 | LCMS (AM3): rt = 0.677 min, (482.3 [M + H]⁺), 100% purity. ¹H NMR (400 MHz, MeOH-d₄) δ: 10.03 (s, 1H), 8.86 (d, J = 5.6 Hz, 1H), 8.68-8.66 (d, 1H), 8.26 (d, J = 1.6 Hz, 1H), 8.20 (d, J = 5.6 Hz, 1H), 7.87 (dd, J = 8.4, 1.6 Hz, 1H), 7.54 (d, J = 8.0 Hz, |
| | 8- | (compound | 1H), 7.39 (d, J = 8.8 Hz, 1H), 7.20-7.13 |
| | carboxamide | 1.119) | (t, 1H), 7.09-7.05 (t, 1H), 6.63 (s, 1H), |
| | | | 4.39 (s, 2H), 4.05 (t, J = 4.8 Hz, 2H), |
| | | | 3.48-3.42 (m, 2H), 3.28-3.22 (m, 2H), |
| | | | 3.13-3.07 (m, 2H), 1.92-1.78 (m, 4H) |
| | | | ppm. |
| | | | Purification Method PM107 |
| EXAMPLE 13 | 5-((2-((4-(((5- Chloro-1H- indol-2- yl)methyl)amino) butyl)amino) ethyl)amino) benzo[c][2,6] naphthyridine- 8- | 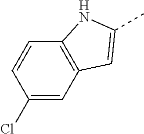 | ¹H NMR (400 MHz, MeOH-d₄) δ: 10.03 (s, 1H), 8.86 (d, J = 5.6 Hz, 1H), 8.68 (d, J = 8.4 Hz, 1H), 8.26 (d, J = 1.6 Hz, 1H), 8.18 (d, J = 5.6 Hz, 1H), 7.87 (dd, J = 8.4, 1.6 Hz, 1H), 7.53 (d, J = 1.6 Hz, 1H), 7.37 (d, J = 8.8 Hz, 1H), 7.14 (dd, J = 8.8, 2.0 Hz, 1H), 6.62 (s, 1H), 4.39 (s, 2H), 4.05 (t, J = 5.2 Hz, 2H), 3.46-3.42 (t, 2H), 3.30-3.23 (m, 2H), 3.15-3.11 (m, |
| | carboxamide | (compound | 2H), 1.93-1.76 (br m, 4H) ppm. |
| | | 1.120) | LCMS (AM3): rt = 0.707 min, (516.3 |
| | | | [M + H]⁺), 100% purity. |
| | | | Purification Method PM107 |
| EXAMPLE 14 | 5-((2-((4-(((4- Methyl-3- phenyl-1H- pyrazol-5- yl)methyl)amino) butyl)amino) ethyl)amino) benzo[c][2,6] naphthyridine- | 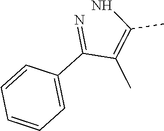 | LCMS (AM3): rt = 0.711 min, (523.4 [M + H]⁺), 100% purity. ¹H NMR (400 MHz, MeOH-d₄) δ: 10.05 (s, 1H), 8.89 ( d, J = 6.0 Hz, 1H), 8.69 (d, J = 8.4 Hz, 1H), 8.33-8.32 (d, 1H), 8.29-8.28 (d, 1H),7.92 (dd, J = 1.6, 8.4 Hz, 1H), 7.58-7.41 (m, 5H), 4.27 (s, 2H), 4.12-4.08 (t, 2H), 3.54-3.49 (t, 2H), 3.29 (t, J = 7.2 Hz, 2H), 3.20 (t, J = 7.2 Hz, |
| | 8- | (compound | 2H), 2.23 (s, 3H), 1.98-1.82 (br m, 4H) |
| | carboxamide | 1.142) | ppm |
| | | | Purification Method PM108 |
| EXAMPLE 15 | 5-((2-((4-(((4- chloro-3- phenyl-1H- pyrazol-5- yl)methyl)amino) butyl)amino) ethyl)amino) benzo[c][2,6] | 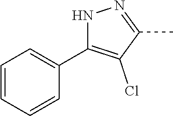 | ¹H NMR (400 MHz, MeOH-d₄) δ: 10.03- 9.95 (m, 1H), 8.88 (d, J = 5.6 Hz, 1H), 8.70-8.55 (m, 1H), 8.32-8.22 (m, 2H), 7.93-7.83 (m, 1H), 7.74 (d, J = 8.0 Hz, 2H), 7.58-7.42 (m, 3H), 4.32 (s, 2H), 4.12-4.08 (m, 2H), 3.55-3.50 (m, 2H), 3.33-3.19 (m, 4H), 1.96-1.82 (m, 4H) ppm. |
| | naphthyridine- | (compound | LCMS (AM3): rt = 0.695 min, (543.1 |
| | 8- | 1.110) | [M + H]⁺), 100% purity. |
| | carboxamide | | Purification Method PM109 |
| EXAMPLE 16 | 5-((2-((4-(((3- Phenyl-1H- pyrazol-5- yl)methyl)amino) butyl)amino) ethyl)amino) benzo[c][2,6] naphthyridine- 8- | 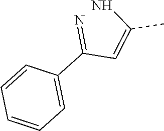 | ¹H NMR (400 MHz, MeOH-d₄) δ: 9.99 (s, 1H), 8.87 (d, J = 5.6 Hz, 1H), 8.62 (d, J = 8.8 Hz, 1H), 8.29-8.26 (m, 2H), 7.88 (dd, J = 1.6, 8.4 Hz, 1H), 7.70-7.67 (d, 2H), 7.45-7.33 (m, 3H), 6.76 (s, 1H), 4.28 (s, 2H), 4.10 (t, J = 5.2 Hz, 2H), 3.51 (t, J = 5.2 Hz, 2H), 3.27 (t, J = 6.8 Hz, 2H), 3.16 (t, J = 6.8 Hz, 2H), 1.95-1.82 (br m, 4H) ppm. |
| | carboxamide | (compound | LCMS (AM3): rt = 0.686 min, (509.2 |
| | | 1.143) | [M + H]⁺), 100% purity. |
| | | | Purification Method PM110 |
| EXAMPLE 17 | 5-((2-((4-(((5- Fluoro-1H- indol-2- yl)methyl)amino) butyl)amino) ethyl)amino) benzo[c][2,6] naphthyridine- 8- | 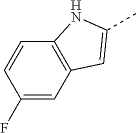 | ¹H NMR (400 MHz, DMSO-d₆) δ: 11.39 (s, 1H), 10.13 (s, 1H), 9.12 (br s, 1H), 8.92 (d, J = 5.6 Hz, 1H), 8.77 (d, J = 8.4 Hz, 2H), 8.28-8.18 (m, 3H), 7.88 (dd, J = 1.6, 8.4 Hz, 1H), 7.50 (s, 1H), 7.47-7.40 (m, 1H), 7.35-7.23 (m, 2H), 6.99 (dt, J = 2.8, 9.2 Hz, 1H), 6.59 (d, J = 1.2 Hz, 1H), 4.32 (s, 2H), 3.95-3.88 (m, 2H), 3.39-3.32 (m, 2H), 3.10-2.90 |
| | carboxamide | (compound | (m, 4H), 1.72-1.60 (m, 4H) ppm. |
| | | 1.111) | LCMS (AM3): rt = 0.714 min, (500.3 |
| | | | [M + H]⁺), 96.4% purity. |
| | | | Purification Method PM109 |
| EXAMPLE 18 | 5-((2-((4-((3- Chloro-4- fluorobenzyl) amino)butyl) amino)ethyl) | 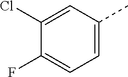 | ¹H NMR (400 MHz, MeOH-d₄) δ: 10.04 (s, 1H), 8.87 (d, J = 5.8 Hz, 1H), 8.69 (d, J = 8.5 Hz, 1H), 8.28 (d, J = 1.8 Hz, 1H), 8.23 (d, J = 5.8 Hz, 1H), 7.90 (dd, J = 1.8, 8.4 Hz, 1H), 7.67 (dd, J = 2.2, 6.9 Hz, |
| | amino)benzo | (compound | 1H), 7.49-7.46 (m, 1H), 7.36-7.31 |
| | [c][2,6]naph- | 1.205) | (2 × d, 1H), 4.20 (s, 2H), 4.07 (t, J = 5.3 Hz, |
| | thyridine-8- | | 2H), 3.48-3.46 (t, 2H), 3.26 ( t, J = |
| | carboxamide | | 7.2 Hz, 2H), 3.11 (t, J = 7.3 Hz, 2H), |
| | | | 1.93-1.78 (m, 4H) ppm. |
| | | | LCMS (AM3): rt = 0.685 min, (495.1 |
| | | | [M + H]⁺), 99.8% purity. |
| | | | Purification Method PM111 |
| EXAMPLE 19 | 5-((2-((4-((3- Chloro-4- (trifluorometh- oxy)benzyl) amino)butyl) amino)ethyl) amino)benzo [c][2,6]naph- thyridine-8- | 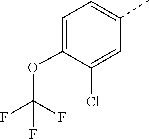 | ¹H NMR (400 MHz, MeOH-d₄) δ: 10.03 (s, 1H), 8.87 (d, J = 5.8 Hz, 1H), 8.71 (d, J = 8.5 Hz, 1H), 8.26 (d, J = 1.8 Hz, 1H), 8.16 (d, J = 5.8 Hz, 1H), 7.89 (dd, J = 1.8, 8.4 Hz, 1H), 7.78 (d, J = 1.5 Hz, 1H), 7.60-7.56 (m, 2H), 4.26 (s, 2H), 4.06-4.03 (t, 2H), 3.46-3.44 (t, 2H), 3.29-3.27 (m, 2H), 3.16-3.13 (m, 2H), 1.95-1.79 (m, 4H) ppm. |
| | carboxamide | (compound | LCMS (AM3): rt = 0.735 min, (561.1 |
| | | 1.103) | [M + H]⁺), 99.77% purity. |
| | | | Purification Method PM112 |
| EXAMPLE 20 | 5-((2-((4-(((6- Chloro-1H- indol-2- yl)methyl)amino) butyl)amino) ethyl)amino) benzo[c][2,6] naphthyridine- | 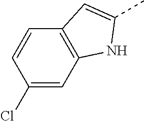 | ¹H NMR (400 MHz, MeOH-d₄) δ: 10.04 (s, 1H), 8.88 (d, J = 5.6 Hz, 1H), 8.71 (d, J = 8.4 Hz 1H), 8.28 (d, J = 1.6 Hz, 1H), 8.18 (d, J = 5.6 Hz, 1H), 7.89 (dd, J = 8.4, 1.6 Hz, 1H), 7.53 (d, J = 8.4 Hz, 1H), 7.43 (s, 1H), 7.06 (dd, J = 8.4, 1.6 Hz, 1H), 6.67 (s, 1H), 4.41 (s, 2H), 4.07-4.04 (t, 2H), 3.47-3.44 (t, 2H), 3.26-3.23 (t, |
| | 8- | (compound | 2H), 3.19-3.16 (t, 2H), 1.95-1.83 (m, |
| | carboxamide | 1.168) | 4H) ppm. |
| | | | LCMS (AM3): rt = 0.747 min, (516.2 |
| | | | [M + H]⁺), 100% purity. |
| | | | Purification Method PM113 |
| EXAMPLE 21 | 5-((2-((4-(((6- Fluoro-1H- indol-2- yl)methyl)amino) butyl)amino) ethyl)amino) benzo[c][2,6] naphthyridine- | 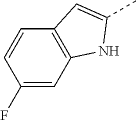 | ¹H NMR (400 MHz, MeOH-d₄) δ: 9.96 (s, 1H), 8.85 (d, J = 6.0 Hz, 1H), 8.59 (d, J = 8.4 Hz, 1H), 8.25-8.24 (d, 1H), 8.23-8.22 (d, 1H), 7.84 (dd, J = 2.0, 8.4 Hz, 1H), 7.49 (dd, J = 5.2, 8.8 Hz, 1H), 7.08 (dd, J = 2.4, 10.0 Hz, 1H), 6.86-6.80 (td, 1H), 6.63 (s, 1H), 4.37 (s, 2H), 4.07 (t, J = 5.2 Hz, 2H), 3.48 (t, J = |
| | 8- | (compound | 5.2 Hz, 2H), 3.24 (t, J = 7.2 Hz, 2H), 3.11 |
| | carboxamide | 1.137) | (t, J = 7.2 Hz, 2H), 1.92-1.80 (br m, 4H) |
| | | | ppm. |
| | | | LCMS (AM3): rt = 0.723 min, (500.2 |
| | | | [M + H]⁺), 100% purity. |
| | | | Purification Method PM114 |
| EXAMPLE 22 | 5-((2-((4-(((5- Methoxy-1H- indol-2- yl)methyl)amino) butyl)amino) ethyl)amino) benzo[c][2,6] naphthyridine- 8- | 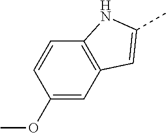 | ¹H NMR (400 MHz, MeOH-d₄) δ: 9.95 (s, 1H), 8.84 (d, J = 5.6 Hz, 1H), 8.57 (d, J = 8.8 Hz, 1H), 8.24 (d, 1H), 8.22-8.21 (d, 1H), 7.83 (dd, J = 1.6, 8.8 Hz, 1H), 7.25 (d, J = 8.8 Hz, 1H), 7.00 (d, J = 2.4 Hz, 1H), 6.80 (dd, J = 2.4, 8.8 Hz, 1H), 6.54 (s, 1H), 4.35 (s, 2H), 4.06 (t, J = 5.6 Hz, 2H), 3.78 (s, 3H), 3.47 (t, J = 5.6 Hz, 2H), 3.22 ( t, J = 6.8 Hz, 2H), 3.10 ( t, J = 6.8 Hz, |
| | carboxamide | (compound | 2H), 1.92-1.78 (br m, 4H) ppm |
| | | 1.145) | LCMS (AM3): rt = 0.711 min, (512.3 |
| | | | [M + H]⁺), 100% purity. |
| | | | Purification Method PM115 |
| EXAMPLE 23 | 5-((2-((4-(((4- methyl-5- phenylpyridin- 2- yl)methyl)amino) butyl)amino) ethyl)amino) benzo[c][2,6] | 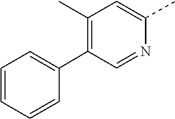 | ¹H NMR (400 MHz, MeOH-d₄) δ: 10.04 (s, 1H), 8.88 (d, J = 5.6 Hz, 1H), 8.70 (d, J = 8.4 Hz, 1H), 8.37 (s, 1H), 8.30 (d, J = 1.6 Hz, 1H), 8.21 (d, J = 5.6 Hz, 1H), 7.90 (dd, J = 8.4 Hz, 1.6 Hz, 1H), 7.53-7.41 (m, 3H), 7.38 (s, 1H), 7.34-7.32 (d, 2H), 4.37 (s, 2H), 4.07 (t, J = 5.2 Hz, 2H), 3.48 (t, J = 5.2 Hz, 2H), |
| | naphthyridine- | (compound | 3.30-3.26 (m, 2H), 3.20 (t, J = 7.6 Hz, |
| | 8- | 1.167) | 2H), 2.33 (s, 3H), 1.98-1.84 (m, 4H) |
| | carboxamide | | ppm. |
| | | | LCMS (AM3): rt = 0.734 min, (534.3 |
| | | | [M + H]⁺), 100% purity. |
| | | | Purification Method PM42 |
| EXAMPLE 24 | 5-((2-((4-(((5- (Trifluoromethyl)- 1H-indol- 2- yl)methyl)amino) butyl)amino) ethyl)amino) benzo[c][2,6] naphthyridine- 8- carboxamide | 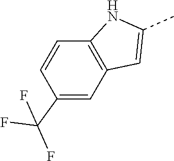 | ¹H NMR (400 MHz, MeOH-d₄) δ: 10.00 (s, 1H), 8.86 (d, J = 5.6 Hz, 1H), 8.69-8.48 (m, 1H), 8.28-8.24 (m, 2H), 7.88-7.80 (m, 2H), 7.54 (d, J = 8.4 Hz, 1H), 7.42-7.40 (m, 1H), 6.78 (s, 1H), 4.43 (s, 2H), 4.01 (m, 2H), 3.49 (m, 2H), 3.24 (m, 2H), 3.16-3.13 (m, 2H), 1.90-1.80 (m, 4H) ppm. LCMS (AM3): rt = 0.727 min, (550.2 [M + H]⁺), 100% purity. Purification Method PM116 |
| | | (compound | |
| | | 1.76) | |
| EXAMPLE 25 | 5-((2-((4-(((2- Methoxy- [1,1′- biphenyl]-4- yl)methyl)amino) butyl)amino) ethyl)amino) benzo[c][2,6] | 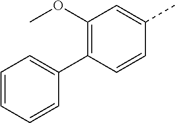 | ¹H NMR (400 MHz, MeOH-d₄) δ: 10.05 (s, 1H), 8.88 (d, J = 5.6 Hz, 1H), 8.71 (d, J = 8.4 Hz, 1H), 8.30 (d, J = 1.6 Hz, 1H), 8.21 (d, J = 5.6 Hz, 1H), 7.90 (dd, J = 1.6, 8.0 Hz, 1H), 7.46-7.44 (m, 2H), 7.40-7.29 (m, 4H), 7.21 (s, 1H), 7.13 (dd, J = 1.6, 7.8 Hz, 1H), 4.25 (s, 2H), 4.07 (t, J = 5.2 Hz, 2H), 3.83 (s, 3H), 3.47 |
| | naphthyridine- | (compound | (t, J = 5.2 Hz, 2H), 3.30-3.25 (m, 2H), |
| | 8- | 1.314) | 3.14 (t, J = 5.2 Hz, 2H), 1.96-1.80 (m, |
| | carboxamide | | 4H) ppm. |
| | | | LCMS (AM3): rt = 0.751 min, (549.4 |
| | | | [M + H]⁺), 100% purity. |
| | | | Purification Method PM117 |
| EXAMPLE 26 | 5-((2-((4-(((2- Cyano-[1,1′- biphenyl]-4- yl)methyl)amino) butyl)amino) ethyl)amino) benzo[c][2,6] naphthyridine- 8- | 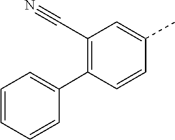 | ¹H NMR (400 MHz, MeOH-d₄) δ: 10.03 (s, 1H), 8.87 (d, J = 5.6 Hz, 1H), 8.71 (d, J = 8.2 Hz, 1H), 8.27 (s, 1H), 8.15 (d, J = 5.6 Hz, 1H), 7.99 (s, 1H), 7.93-7.87 (m, 2H), 7.70 (d, J = 8.0 Hz, 1H), 7.60- 7.48 (m, 5H), 4.34 (s, 2H), 4.05 (t, J = 4.6 Hz, 2H), 3.45 (t, J = 4.8 Hz, 2H), 3.46-3.44 (m, 2H), 3.18 (d, J = 7.6 Hz, 2H), 1.96-1.85 (m, 4H) ppm. |
| | carboxamide | (Compound | LCMS (AM3): rt = 0.750 min, (544.4 |
| | | 1.317) | [M + H]⁺), 100% purity. |
| | | | Purification Method PM44 |
| EXAMPLE 27 | 5-((2-((4-(((2- Fluoro-[1,1′- biphenyl]-4- yl)methyl)amino) butyl)amino) ethyl)amino) benzo[c][2,6] naphthyridine- | 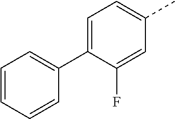 | ¹H NMR (400 MHz, MeOH-d₄) δ: 10.02 (s, 1H), 8.87 (d, J = 5.9 Hz, 1H), 8.68 (d, J = 8.3 Hz, 1H), 8.29 (d, J = 1.7 Hz, 1H), 8.21 (d, J = 5.9 Hz, 1H), 7.89 (dd, J = 1.8, 8.4 Hz, 1H), 7.60-7.34 (m, 8H), 4.26 (s, 2H), 4.07 (t, J = 5.3 Hz, 2H), 3.48 (t, J = 5.3 Hz, 2H), 3.29-3.24 (t, 2H), 3.14 (t, J = 7.3 Hz, 2H), 1.95-1.80 (br m, 4H) |
| | 8- | (Compound | ppm. |
| | carboxamide | 1.131) | LCMS (AM3): rt = 0.733 min, (537.3 |
| | | | [M + H]⁺), 100% purity. |
| | | | Purification Method PM116 |
| EXAMPLE 28 | 5-((2-((4-(((2- Chloro-2′- (cyanomethyl)- [1,1′- biphenyl]-4- yl)methyl)amino) butyl)amino) ethyl)amino) | 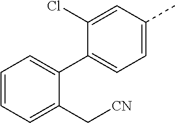 | ¹H NMR (400 MHz, MeOH-d₄) δ: 10.03 (s, 1H), 8.87 (d, J = 5.6 Hz, 1H), 8.71 (d, J = 8.8 Hz, 1H), 8.28 (d, J = 2.0 Hz, 1H), 8.17 (d, J = 5.6 Hz, 1H), 7.90 (dd, J = 8.4 Hz, 1.6 Hz, 1H), 7.74 (d, J = 1.6 Hz, 1H), 7.59-7.41 (m, 5H), 7.23-7.19 (m, 1H), 4.30 (s, 2H), 4.06 (t, J = 5.2 Hz, 2H), 3.75-3.55 (dd, 2H), 3.46 (t, J = 5.2 Hz, |
| | benzo[c][2,6] | (Compound | 2H), 3.30-3.25 (m, 2H), 3.20-3.14 (m, |
| | naphthyridine- | 1.105) | 2H), 1.98-1.82 (m, 4H) ppm. |
| | 8- | | LCMS (AM3): rt = 0.771 min, (592.2 |
| | carboxamide | | [M + H]⁺), 100% purity. |
| | | | Purification Method PM116 |
| EXAMPLE 29 | 5-((2-((4-((3- Chloro-4- (pyrazin-2- yl)benzyl)amino) butyl)amino) ethyl)amino) benzo[c][2,6] naphthyridine- | 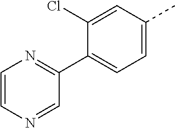 | ¹H NMR (400 MHz, MeOH-d₄) δ: 10.01 (s, 1H), 8.93-8.86 (m, 2H), 8.74 (s, 1H), 8.65- 8.61 (m, 2H), 8.30-8.25 (m, 2H), 7.88 (dd, J = 8.4 Hz, 1.6 Hz, 1H), 7.77 (s, 1H), 7.73- 7.69 (d, 1H), 7.60 (dd, J = 8.0 Hz, 1.2 Hz, 1H), 4.30 (s, 2H), 4.09 (t, J = 5.2 Hz, 2H), 3.50 (t, J = 5.2 Hz, 2H), 3.27 (t, J = 7.2 Hz, 2H), 3.16 (t, J = 7.2 Hz, 2H), 1.96-1.81 (m, |
| | 8- | (Compound | 4H) ppm. |
| | carboxamide | 1.220) | LCMS (AM3): rt = 0.706 min, (555.2 |
| | | | [M + H]⁺), 100% purity. |
| | | | Purification Method PM118 |
| EXAMPLE 30 | 5-((2-((4-(((5- Chloro-6- phenylpyridin- 3- yl)methyl)amino) butyl)amino) ethyl)amino) benzo[c][2,6] | 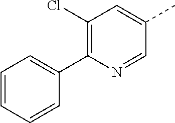 | ¹H NMR (400 MHz, MeOH-d₄) δ: 10.03 (s, 1H), 8.87 (d, J = 5.6 Hz, 1H), 8.70-8.68 (m, 2H), 8.28 (d, J = 1.6 Hz, 1H), 8.20 (d, J = 5.6 Hz, 1H), 8.17 (d, J = 2.0 Hz, 1H), 7.89 (dd, J = 8.4 Hz, 2.0 Hz, 1H), 7.70-7.64 (m, 2H), 7.54-7.46 (m, 3H), 4.35 (s, 2H), 4.07 (t, J = 5.2 Hz, 2H), 3.47 (t, J = 5.2 Hz, 2H), 3.31-3.26 |
| | naphthyridine- | (Compound | (m, 2H), 3.23-3.17 (m, 2H), 1.98-1.83 |
| | 8- | 1.127) | (m, 4H) ppm. |
| | carboxamide | | LCMS (AM3): rt = 0.734 min, (554.2 |
| | | | [M + H]⁺), 100% purity. |
| | | | Purification Method PM119 |
| EXAMPLE 32 | 5-((2-((4-(((6- Chloroquinolin- 2- yl)methyl)amino) butyl)amino) | 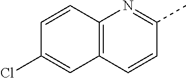 | ¹H NMR (400 MHz, MeOH-d₄) δ: 9.95 (s, 1H), 8.87 (d, J = 6.0 Hz, 1H), 8.53 (d, J = 8.8 Hz, 1H), 8.34-8.25 (m, 3H), 8.02 (d, J = 9.2 Hz, 1H), 7.96 (d, J = 2.4 Hz, 1H), 7.82 (dd, J = 1.6, 8.4 Hz, 1H), 7.71 |
| | ethyl)amino) | (Compound | (dd, J = 2.4, 8.8 Hz, 1H), 7.53 (d, J = 8.8 Hz, |
| | benzo[c][2,6] | 1.185) | 1H), 4.60 (s, 2H), 4.13 (t, J = 5.6 Hz, |
| | naphthyridine- | | 2H), 3.55 (t, J = 5.6 Hz, 2H), 3.33 (td, J = |
| | 8- | | 1.6, 3.2 Hz, 2H), 3.34-3.27 (br m, 2H), |
| | carboxamide | | 2.07-1.90 (m, 4H) ppm. |
| | | | LCMS (AM3): rt = 0.666 min, (528.1 |
| | | | [M + H]⁺), 99.5% purity. |
| | | | Purification Method PM42 |
| EXAMPLE 33 | 5-((2-((4-(((6- (Trifluoromethyl) quinolin-2- yl)methyl)amino) butyl)amino) ethyl)amino) | 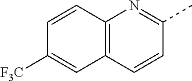 | ¹H NMR (400 MHz, MeOH-d₄) δ: 10.04 (s, 1H), 8.89 (d, J = 5.6 Hz, 1H), 8.70 (d, J = 8.4 Hz, 1H), 8.57 (d, J = 8.8 Hz, 1H), 8.41 (s, 1H), 8.32 (d, J = 1.6 Hz, 1H), 8.26-8.22 (m, 2H), 7.99 (dd, J = 2.0, 8.8 Hz, 1H), 7.88 (dd, J = 1.6, 8.4 Hz, |
| | benzo[c][2,6] | (Compound | 1H), 7.65 (d, J = 8.8 Hz, 1H), 4.68 (s, 2H), |
| | naphthyridine- | 1.144) | 4.10 (t, J = 5.2 Hz, 2H), 3.53-3.50 (t, |
| | 8- | | 2H), 3.34-3.28 (m, 4H), 2.08-1.88 (m, |
| | carboxamide | | 4H) ppm |
| | | | LCMS (AM3): rt = 0.690 min, (562.1 |
| | | | [M + H]⁺), 99.2% purity. |
| | | | Purification Method PM117 |
| EXAMPLE 34 | 5-((2-((4-(((5- Phenylthiazol- 2- yl)methyl)amino) butyl)amino) | 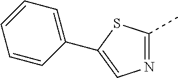 | ¹H NMR (400 MHz, MeOH-d₄) δ: 9.95 (s, 1H), 8.81 (d, J = 5.8 Hz, 1H), 8.63 (d, J = 8.5 Hz, 1H), 8.25 (d, J = 1.8 Hz, 2H), 8.10 (d, J = 5.6 Hz, 1H), 7.89 (s, 1H), 7.86 (dd, J = 1.9, 8.4 Hz, 1H), 7.51-7.48 |
| | ethyl)amino) | (Compound | (m, 2H), 7.40-7.32 (m, 3H), 4.24 (s, 2H), |
| | benzo[c][2,6] | 1.258) | 4.03 (t, J = 5.3 Hz, 2H), 3.46 (t, J = 5.3 Hz, |
| | naphthyridine- | | 2H), 3.23 (t, J = 6.7 Hz, 2H), 2.93 (t, |
| | 8- | | J = 6.7 Hz, 2H), 1.91-1.76 (m, 4H) ppm. |
| | carboxamide | | LCMS (AM3): rt = 0.700 min, (526.3 |
| | | | [M + H]⁺), 100% purity. |
| | | | Purification Method PM121 |
| EXAMPLE 35 | 5-((2-((4-((3- Chloro-4- cyclobutoxy- benzyl)amino) butyl)amino) ethyl)amino) benzo[c][2,6] naphthyridine- 8- carboxamide | 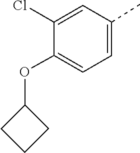 | ¹H NMR (400 MHz, MeOH-d₄) δ: 10.03 (s, 1H), 8.86 (d, J = 5.6 Hz, 1H), 8.71 (d, J = 8.5 Hz, 1H), 8.26 (d, J = 1.8 Hz, 1H), 8.17 (d, J = 5.8 Hz, 1H), 7.90 (dd, J = 1.9, 8.4 Hz, 1H), 7.53 (d, J = 2.3 Hz, 1H), 7.34 (dd, J = 2.2, 8.5 Hz, 1H), 6.96 (d, J = 8.5 Hz, 1H), 4.77 (quintet, J = 7.0 Hz, 1H), 4.13 (s, 2H), 4.06-4.03 (t, 2H), 3.47-3.45 (t, 2H), 3.29-3.25 (t, 2H), 3.08 (t, J = 7.4 Hz, 2H), 2.54-2.46 (m, 2H), |
| | | (Compound | 2.21-2.13 (m, 2H), 1.92-1.70 (m, 6H) |
| | | 1.121) | ppm. |
| | | | LCMS (AM3): rt = 0.735 min, (547.1 |
| | | | [M + H]⁺), 100% purity |
| | | | Purification Method PM122 |
| EXAMPLE 36 | 5-((2-((4-((3- Cyano-4- cyclobutoxy- benzyl)amino) butyl)amino) ethyl)amino) benzo[c][2,6] naphthyridine- 8- carboxamide | 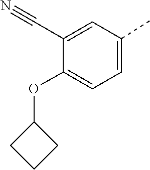 | ¹H NMR (400 MHz, MeOH-d₄) δ: 10.03 (s, 1H), 8.87 (d, J = 5.6 Hz, 1H), 8.67 (d, J = 8.8 Hz, 1H), 8.28 (d, J = 1.6 Hz, 1H), 8.23 (d, J = 6.0 Hz, 1H), 7.89 (dd, J = 1.6, 8.4 Hz, 1H), 7.75 (d, J = 2.2 Hz, 1H), 7.70 (dd, J = 2.0, 8.8 Hz, 1H), 7.07 (d, J = 8.8 Hz, 1H), 4.85-4.82 (m, 1H), 4.17 (s, 2H), 4.07 (t, J = 5.2 Hz, 2H), 3.47 (t, J = 5.2 Hz, 2H), 3.26 (t, J = 7.2 Hz, 2H), 3.09 (t, J = 7.2 Hz, 2H), 2.57-2.48 (m, 2H), 2.25-2.14 (m, 2H), 1.95-1.71 (m, |
| | | (Compound | 6H) ppm |
| | | 1.327) | LCMS (AM3): rt = 0.723 min, (538.2 |
| | | | [M + H]⁺), 100% purity. |
| | | | Purification Method PM119 |
| EXAMPLE 37 | 5-((2-((4-((3- Cyano-4- (trifluoromethoxy) benzyl) amino)butyl) amino)ethyl) amino)benzo [c][2,6]naph- thyridine-8- carboxamide | 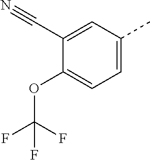 | ¹H NMR (400 MHz, MeOH-d₄) δ: 10.02 (s, 1H), 8.86 (d, J = 6.0 Hz, 1H), 8.69 (d, J = 8.4 Hz, 1H), 8.27 (d, J = 1.6 Hz, 1H), 8.20 (d, J = 6.0 Hz, 1H), 8.03 (d, J = 2.0 Hz, 1H), 7.95 (dd, J = 2.0, 8.8 Hz, 1H), 7.89 (dd, J = 2.0, 8.8 Hz, 1H), 7.66 (dd, J = 1.2, 8.6 Hz, 1H), 4.31 (s, 2H), 4.06 (t, J = 5.2 Hz, 2H), 3.46 (t, J = 5.2 Hz, 2H), 3.30-3.26 (t, 2H), 3.16 (t, J = 7.2 Hz, 2H), 1.96-1.80 (br m, 4H) ppm. |
| | | (Compound | LCMS (AM3): rt = 0.717 min, (552.3 |
| | | 1.370) | [M + H]⁺), 100% purity. |
| | | | Purification Method PM123 |

### Example 9

### 5-((2-((4-(((2-Chloro-2′-(hydroxymethyl)-[1,1′-biphenyl]-4-yl)methyl)amino)butyl)amino)ethyl)amino)benzo[c][2,6]naphthyridine-8-carboxamide

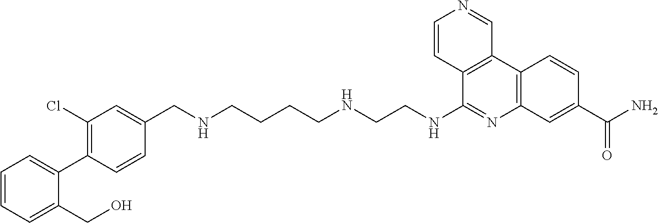 [SMILES: NC(=O)c1ccc2c(c1)nc(NCCNCCCCNCc1ccc(-c3ccccc3CO)c(Cl)c1)c1ccncc12]

A mixture of compound 1.201 (88 mg, 0.103 mmol, TFA salt) and TFA (67.53 mmol, 5 mL) in DCM (5 mL) was stirred at RT for 1 h. The reaction mixture was concentrated in vacuo and the residue was purified (PM42) to afford EXAMPLE 9 (23.9 mg, 28.7% yield, TFA salt) as a yellow gum.

LCMS (AM3): rt=0.745 min, (583.1 [M+H]⁺), 100% purity.

¹H NMR (400 MHz, MeOH-d₄) δ: 10.04 (br s, 1H), 8.90 (br s, 1H), 8.65 (d, J=8.4 Hz, 1H), 8.36-8.31 (m, 2H), 7.89 (dd, J=1.2, 8.4 Hz, 1H), 7.68 (d, J=1.2 Hz, 1H), 7.60 (d, J=7.6 Hz, 1H), 7.49 (dd, J=1.6, 8.0 Hz, 1H), 7.45-7.41 (t, 1H), 7.39-7.33 (m, 2H), 7.09 (d, J=6.8 Hz, 1H), 4.43-4.26 (2×d, 4H), 4.11 (t, J=5.2 Hz, 2H), 3.51 (t, J=5.2 Hz, 2H), 3.26 (t, J=6.8 Hz, 2H), 3.16 (t, J=6.8 Hz, 2H), 1.94-1.82 (br m, 4H) ppm.

### Example 10

### 5-((2-((4-((3-Chloro-4-(pyridin-3-yl)benzyl)amino)butyl)amino)ethyl)amino)benzo[c][2,6]naphthyridine-8-carboxamide

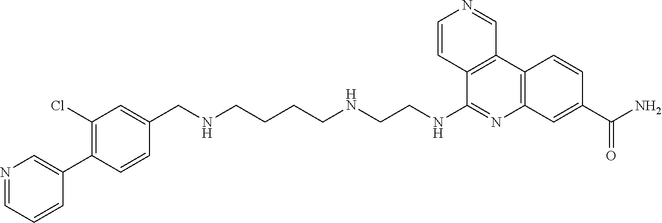 [SMILES: NC(=O)c1ccc2c(c1)nc(NCCNCCCCNCc1ccc(-c3cccnc3)c(Cl)c1)c1ccncc12]

A mixture of compound 1.203 (80 mg, 0.104 mmol, TFA salt) and TFA (5 mL, 0.068 mmol) in DCM (5 mL) was stirred at RT for 1 h. The reaction mixture was concentrated in vacuo and the residue was purified (PM105) to afford EXAMPLE 10 (49.15 mg, 60.4% yield, TFA salt) as a yellow solid.

LCMS (AM3): rt=0.646 min, (554.1 [M+H]⁺), 100% purity.

¹H NMR (400 MHz, MeOH-d₄) δ: 10.04 (s, 1H), 8.93-8.83 (m, 3H), 8.66 (d, J=8.8 Hz, 1H), 8.49 (d, J=8.0 Hz, 1H), 8.32 (s, 1H), 8.32-8.29 (d, 1H), 8.00 (td, J=8.0 Hz, 5.6 Hz, 1H), 7.90 (dd, J=8.4 Hz, 1.6 Hz, 1H), 7.80 (s, 1H), 7.66-7.58 (m, 2H), 4.30 (s, 2H), 4.11 (t, J=5.2 Hz, 2H), 3.51 (t, J=5.2 Hz, 2H), 3.26 (t, J=6.8 Hz, 2H), 3.17 (t, J=6.8 Hz, 2H), 1.96-1.80 (m, 4H) ppm.

### Example 11

### 5-((2-((4-((3-Chloro-4-(pyridin-4-yl)benzyl)amino)butyl)amino)ethyl)amino)benzo[c][2,6]naphthyridine-8-carboxamide

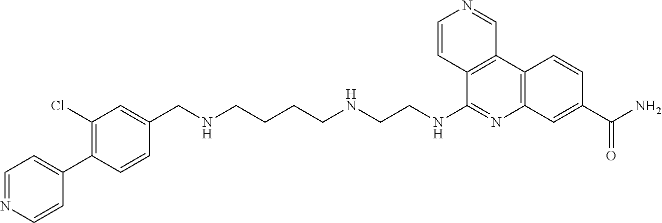 [SMILES: NC(=O)c1ccc2c(c1)nc(NCCNCCCCNCc1ccc(-c3ccncc3)c(Cl)c1)c1ccncc12]

A mixture of compound 1.204 (84 mg, 0.109 mmol, TFA salt) and TFA (5 mL, 67.53 mmol) in DCM (10 mL) was stirred at RT for 1.5 h. The reaction mixture was concentrated in vacuo and the crude product was purified (PM106) to afford EXAMPLE 11 (64.24 mg, 75.1% yield, TFA salt) as a yellow solid.

LCMS (AM3): rt=0.651 min, (554.1 [M+H]⁺), 100% purity.

¹H NMR (400 MHz, MeOH-d₄) δ: 10.05 (s, 1H), 8.90-8.84 (m, 3H), 8.71 (d, J=8.8 Hz, 1H), 8.29 (d, J=1.6 Hz, 1H), 8.20 (d, J=5.6 Hz, 1H), 7.98-7.96 (d, 2H), 7.90 (dd, J=8.4 Hz, 1.6 Hz, 1H), 7.80 (s, 1H), 7.68-7.64 (m, 2H), 4.31 (s, 2H), 4.07 (t, J=5.2 Hz, 2H), 3.47 (t, J=5.2 Hz, 2H), 3.30-3.25 (m, 2H), 3.18 (t, J=7.2 Hz, 2H), 1.96-1.83 (m, 4H) ppm.

### Example 31

### 5-((2-((4-(((2-Chloro-2′-(2-hydroxyethyl)-[1,1′-biphenyl]-4-yl)methyl)amino)butyl)amino)ethyl)amino)benzo[c][2,6]naphthyridine-8-carboxamide

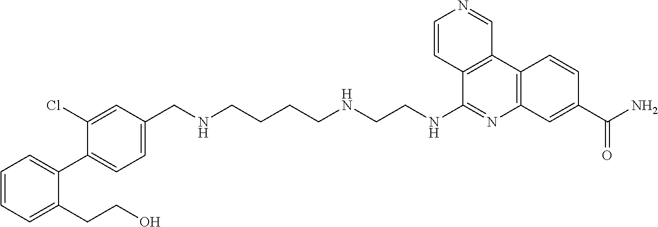 [SMILES: NC(=O)c1ccc2c(c1)nc(NCCNCCCCNCc1ccc(-c3ccccc3CCO)c(Cl)c1)c1ccncc12]

A mixture of compound 1.74 (117 mg, 0.124 mmol, TFA salt) and TFA (27.01 mmol, 2 mL) in DCM (2 mL) was stirred at 30° C. for 2 h. The reaction mixture was concentrated in vacuo and purified (PM120) to afford EXAMPLE 31 (15.55 mg, 18.2% yield, 100% purity, FA salt) as a yellow solid.

LCMS (AM3): rt=0.746 min, (597.2 [M+H]⁺), 100% purity.

¹H NMR (400 MHz, MeOH-d₄) δ: 9.96 (s, 1H), 8.83 (d, J=5.6 Hz, 1H), 8.64 (d, J=8.4 Hz, 1H), 8.47 (br s, 2H), 8.25 (s, 1H), 8.12 (d, J=5.6 Hz, 1H), 7.86 (d, J=8.4 Hz, 1H), 7.64 (s, 1H), 7.46 (d, J=8.0 Hz, 1H), 7.36-7.32 (m, 3H), 7.29-7.24 (m, 1H), 7.04 (d, J=7.6 Hz, 1H), 4.15 (s, 2H), 4.03 (t, J=4.8 Hz, 2H), 3.51 (t, J=7.2 Hz, 2H), 3.43 (t, J=4.8 Hz, 2H), 3.22 (m, 2H), 3.06 (m, 2H), 2.72-2.64 (m, 1H), 2.61-2.54 (m, 1H), 1.86 (m, 4H) ppm.

### Example 38

### 5-((3-(3-(((2-Chloro-[1,1′-biphenyl]-4-yl)methyl)amino)propanamido)propyl)amino)benzo[c][2,6]naphthyridine-8-carboxylic acid

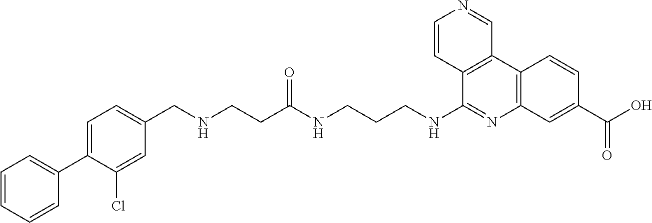 [SMILES: O=C(CCNCc1ccc(-c2ccccc2)c(Cl)c1)NCCCNc1nc2cc(C(=O)O)ccc2c2cnccc12]

To a solution of compound 1.387 (106 mg, 0.151 mmol) in DCM (10 mL) was added TFA (1.30 g, 11.37 mmol) at 20° C. The resulting mixture was stirred at 20° C. for 16 h. The reaction mixture was concentrated in vacuo and the residue was purified (PM104) to afford EXAMPLE 38 (100 mg, 81.6% yield, TFA salt) as a light yellow solid.

LCMS (AM3): rt=0.709 min, (568.1 [M+H]⁺), 98.4% purity.

¹H NMR (400 MHz, MeOH-d₄) δ: 10.07 (s, 1H), 8.97 (d, J=5.6 Hz, 1H), 8.77 (d, J=8.4 Hz, 1H), 8.59 (d, J=1.2 Hz, 1H), 8.37 (d, J=5.6 Hz, 1H), 8.15 (d, J=8.0 Hz, 1H), 7.67 (d, J=1.6 Hz, 1H), 7.50-7.47 (m, 1H), 7.46-7.37 (m, 6H), 4.30 (s, 2H), 3.87 (t, J=6.4 Hz, 2H), 3.42 (t, 2H), 3.38 (t, 2H), 2.78 (t, J=6.4 Hz, 2H), 2.11-2.04 (quin, 2H) ppm.

### Example 39

### 5-((2-((3-(((2-Chloro-[1,1′-biphenyl]-4-yl)methyl)amino)propyl)amino)ethyl)amino)benzo[c][2,6]naphthyridine-8-carboxylic acid

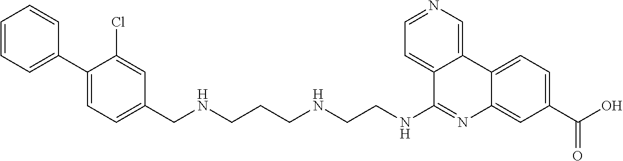 [SMILES: O=C(O)c1ccc2c(c1)nc(NCCNCCCNCc1ccc(-c3ccccc3)c(Cl)c1)c1ccncc12]

To a mixture of compound 1.393 (100 mg, 0.156 mmol) in DCM (10 mL) was added TFA (27.01 mmol) at 20° C. The resulting mixture was stirred at 20° C. for 16 h. The reaction mixture was concentrated in vacuo and the residue was purified (PM124) to afford EXAMPLE 39 (40 mg, 40.6% yield, 2×FA salt) as a light yellow solid.

LCMS (AM1): rt=0.631 min, (540.2 [M+H]⁺), 100% purity.

¹H NMR (400 MHz, MeOH-d₄) δ: 9.94 (s, 1H), 8.81 (d, J=5.6 Hz, 1H), 8.57 (d, J=8.4 Hz, 1H), 8.28 (s, 2H), 8.09 (d, J=5.6 Hz, 1H), 8.00 (d, J=8.0 Hz, 1H), 7.87 (s, 1H), 7.49 (s, 1H), 7.43-7.28 (m, 7H), 4.00 (t, J=4.8 Hz, 2H), 3.97 (s, 2H), 3.43 (t, J=4.8 Hz, 2H), 3.28-3.25 (m, 2H), 3.05 (t, J=7.2 Hz, 2H), 2.18-2.10 (quin, 2H) ppm.

### Example 40

### 6-(3-(3-(((2-Chloro-[1,1′-biphenyl]-4-yl)methyl)amino)propanamido)propyl)-5,6-dihydroimidazo[1,5-c]quinazoline-8-carboxylic acid

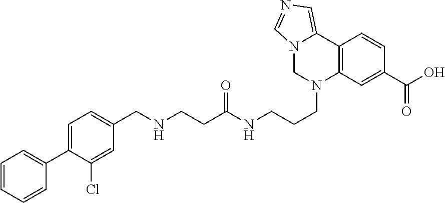 [SMILES: O=C(CCNCc1ccc(-c2ccccc2)c(Cl)c1)NCCCN1Cn2cncc2-c2ccc(C(=O)O)cc21]

A solution of compound 1.807 (90 mg, 0.11 mmol) and TFA (5.00 mL, 67.53 mmol) in DCM (5 mL) was stirred at RT for 1 h. The solution was concentrated in vacuo, followed by addition of EtOH (20 mL) and K₂CO₃ (1 g). The mixture was stirred at RT for 1 h, filtered and concentrated in vacuo. The residue was purified (PM102) to afford EXAMPLE 40 (20 mg, 0.04 mmol, 33.5% yield) as a white solid.

LCMS (AM4): rt=0.610 min, (544.1 [M+H]⁺), 94.8% purity.

¹H NMR (400 MHz, MeOH-d₄) δ: 8.95 (s, 1H), 8.02 (s, 1H), 7.82 (d, J=8.4 Hz, 1H), 7.71-7.68 (m, 3H), 7.53-7.50 (m, 1H), 7.47-7.37 (m, 6H), 5.53 (s, 2H), 4.32 (s, 2H), 3.53 (t, J=7.2 Hz, 2H), 3.38-3.33 (m, 4H), 2.76 (t, J=6.4 Hz, 2H), 1.95-1.88 (m, 2H) ppm.

### Example 41

### 5-((2-((4-(((2-chloro-[1,1′-biphenyl]-4-yl)methyl)amino)butyl)amino)ethyl)amino)benzo[c][2,6]naphthyridine-8-carbonitrile

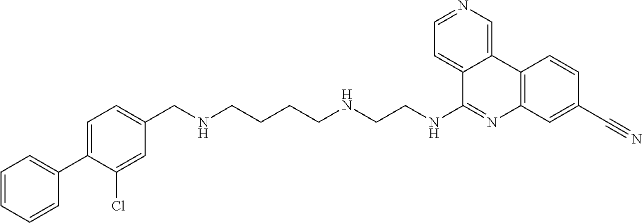 [SMILES: N#Cc1ccc2c(c1)nc(NCCNCCCCNCc1ccc(-c3ccccc3)c(Cl)c1)c1ccncc12]

A solution of compound 1.11 (30 mg, 0.041 mmol) and TFA (1 mL) in DCM (1 mL) was stirred at RT for 4 h. The reaction mixture was concentrated in vacuo and the crude product was purified (PM125) EXAMPLE 41 (11.0 mg, 34% yield, TFA salt) was obtained after lyophilization as a yellow solid.

LCMS (AM1): rt=0.658 min, (535.2 [M+H]⁺), 96.8% purity.

¹H NMR (400 MHz, MeOH-d₄) δ: 10.03 (s, 1H), 8.90 (d, J=6 Hz, 1H), 8.79 (d, J=8.4 Hz, 1H), 8.15 (d, J=4.8 Hz, 1H), 8.10 (d, J=1.6 Hz, 1H), 7.70 (dd, J=8.4 Hz, 1.6 Hz, 1H), 7.67 (s, 1H), 7.48-7.39 (m, 7H), 4.25 (s, 2H), 4.06 (t, J=5.2 Hz, 2H), 3.49-3.47 (m, 2H), 3.20 (t, J=7.2 Hz, 2H), 3.15-3.11 (m, 2H), 1.83 (t, J=3.2 Hz, 4H) ppm.

### Example 42

### N-(5-((2-((4-(((2-chloro-[1,1′-biphenyl]-4-yl)methyl)amino)butyl)amino)ethyl)amino)benzo[c][2,6]naphthyridin-8-yl) acetamide

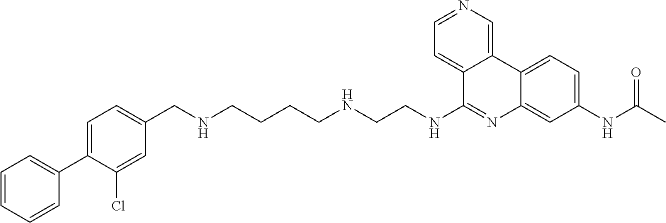 [SMILES: CC(=O)Nc1ccc2c(c1)nc(NCCNCCCCNCc1ccc(-c3ccccc3)c(Cl)c1)c1ccncc12]

A mixture of compound 1.17 (37 mg, 0.033 mmol, TFA salt) and TFA (5 mL, 67.5 mmol) in DCM (10 mL) was stirred at RT for 17 h. The reaction mixture was concentrated in vacuo and the crude product was purified (PM126) to afford EXAMPLE 42 (16.5 mg, 62.5% yield, TFA salt) as a yellow solid.

LCMS (AM3): rt=0.767 min, (567.1 [M+H]⁺), 100% purity.

¹H NMR (400 MHz, MeOH-d₄): δ: 9.94 (s, 1H), 8.78 (d, J=5.6 Hz, 1H), 8.55 (d, J=8.8 Hz, 1H), 8.48 (d, J=2.0 Hz, 1H), 8.21 (d, J=5.6 Hz, 1H), 7.66 (d, J=1.6 Hz, 1H), 7.80-7.39 (m, 7H), 7.29 (dd, J=8.8 Hz, 2.0 Hz, 1H), 4.26 (s, 2H), 4.04 (t, J=5.2 Hz, 2H), 3.46 (t, J=5.2 Hz, 2H), 3.26 (t, J=7.2 Hz, 2H), 3.18 (t, J=7.2 Hz, 2H), 2.16 (s, 3H), 1.99-1.86 (m, 4H) ppm.

### Example 43

### 5-((2-(4-(((2-Chloro-[1,1′-biphenyl]-4-yl)methyl)amino)butanamido)ethyl)amino)benzo[c][2,6]naphthyridine-8-carboxamide

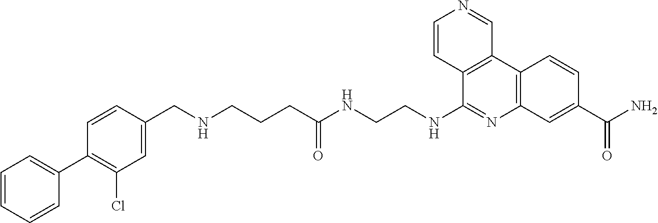 [SMILES: NC(=O)c1ccc2c(c1)nc(NCCNC(=O)CCCNCc1ccc(-c3ccccc3)c(Cl)c1)c1ccncc12]

A mixture of compound 1.222 (160 mg, 0.24 mmol) and TFA (67.53 mmol, 5 mL) in DCM (10 mL) was stirred at RT for 2 h. The reaction mixture was concentrated in vacuo and the residue was dissolved in H₂O (20 mL) to give a solution which was directly lyophilized to afford EXAMPLE 43 (92.98 mg, 56.9% yield, TFA salt) as a yellow solid.

LCMS (AM3): rt=0.766 min, (567.1 [M+H]⁺), 100% purity.

¹H NMR (400 MHz, MeOH-d₄) δ: 10.11 (s, 1H), 9.01 (d, J=1.6 Hz, 1H), 8.81 (d, J=8.8 Hz, 1H), 8.70 (d, J=1.6 Hz, 1H), 8.34 (d, J=5.6 Hz, 1H), 8.10 (d, J=8.8 Hz, 1H), 7.69 (s, 1H), 7.51 (d, J=6.0 Hz, 1H), 7.44-7.48 (m, 6H), 4.33 (s, 2H), 3.93 (t, J=6.8 Hz, 2H), 3.63 (t, J=6.8 Hz, 2H), 3.31-3.28 (m, 2H), 2.57 (t, J=6.8 Hz, 2H), 2.17-2.10 (m, 2H) ppm.

### Example 44

### 5-((3-(3-(((2-Chloro-[1,1′-biphenyl]-4-yl)methyl)amino)propoxy)propyl)amino)benzo[c][2,6]naphthyridine-8-carboxamide

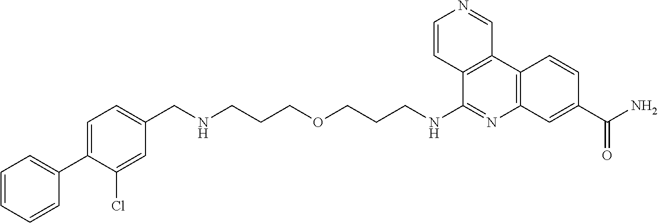 [SMILES: NC(=O)c1ccc2c(c1)nc(NCCCOCCCNCc1ccc(-c3ccccc3)c(Cl)c1)c1ccncc12]

To a mixture of compound 1.117 (125 mg, 0.191 mmol) in DCM (5 mL) was added TFA (1.54 g, 13.51 mmol) at 0° C. The mixture was warmed to 25° C. and stirred for 1 h. The mixture was concentrated in vacuo and purified (PM44) to afford EXAMPLE 44 (70.67 mg, 0.106 mmol, 55.4% yield, TFA salt) as a yellow solid.

LCMS (AM3): rt=0.773 min, (554.1 [M+H]⁺), 100% purity.

¹H NMR (400 MHz, DMSO-d₆) δ: 10.13 (s, 1H), 9.02-8.88 (m, 3H), 8.78 (d, J=8.4 Hz, 1H), 8.42-8.38 (d, 1H), 8.37-8.33 (s, 1H), 8.25-8.20 (s, 1H), 7.92 (d, J=8.4 Hz, 1H), 7.72 (s, 1H), 7.60-7.35 (m, 8H), 4.25-4.20 (m, 2H), 3.75-3.70 (m, 2H), 3.59-3.55 (t, 2H), 3.52-3.48 (t, 2H), 3.10-3.00 (m, 2H), 2.05-1.95 (m, 2H), 1.95-1.85 (m, 2H) ppm.

### Example 45

### 5-((2-(4-(((2-Chloro-[1,1′-biphenyl]-4-yl)methyl)amino)-N-methylbutanamido)ethyl)(methyl)amino)benzo[c][2,6]naphthyridine-8-carboxamide

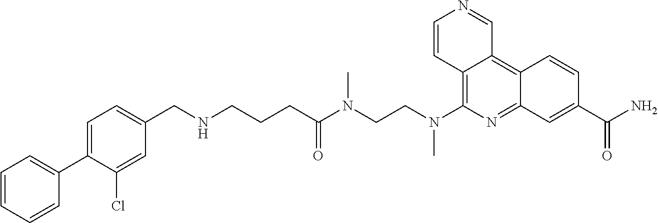 [SMILES: CN(CCN(C)c1nc2cc(C(N)=O)ccc2c2cnccc12)C(=O)CCCNCc1ccc(-c2ccccc2)c(Cl)c1]

To a solution of compound 1.211 (140 mg, 173.00 μmol, TFA salt) in DCM (10 mL) was added TFA (7.70 g, 67.53 mmol) at 25° C. The reaction mixture was stirred at 25° C. for 0.5 h. The reaction mixture was concentrated in vacuo and the residue was purified (PM126) to afford EXAMPLE 45 (48.77 mg, 68.50 μmol, 36.0% yield, TFA salt) as a yellow solid.

LCMS (AM4): rt=0.804 min, (595.2 [M+H]⁺), 99.6% purity.

¹H NMR (400 MHz, DMSO-d₆) δ: 10.19-10.16 (m, 1H), 8.88-8.74 (m, 4H), 8.32-8.23 (m, 2H), 8.09-8.03 (t, 1H), 8.01-7.94 (t, 1H), 7.73 (s, 1H), 7.58-7.41 (m, 7H), 4.21-4.14 (m, 2H), 3.82-3.65 (m, 4H), 3.23 (s, 3H), 3.00-2.84 (m, 5H), 2.62-2.55 (m, 1H), 2.22 (t, J=6.8 Hz, 1H), 1.89-1.67 (m, 2H) ppm.

### Example 46

### 5-((3-(3-(((2-Chloro-[1,1′-biphenyl]-4-yl)methyl)amino)propanamido)propyl)amino)benzo[c][2,6]naphthyridine-8-carboxamide

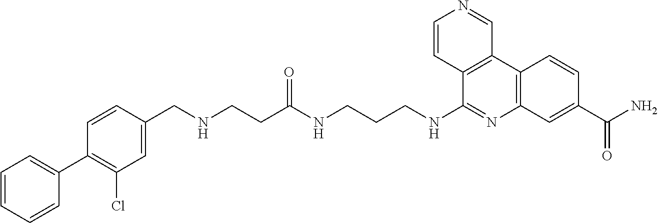 [SMILES: NC(=O)c1ccc2c(c1)nc(NCCCNC(=O)CCNCc1ccc(-c3ccccc3)c(Cl)c1)c1ccncc12]

To a solution of compound 1.234 (150 mg, 225.16 μmol) in DCM (3 mL) was added TFA (1 mL) at 25° C. The reaction mixture was stirred at 25° C. for 0.5 h. The reaction mixture was concentrated in vacuo and the residue was purified (PM44) to afford EXAMPLE 46 (69.51 mg, 102.20 μmol, 45.4% yield, TFA salt) as a yellow solid.

LCMS (AM3): rt=0.782 min, (567.2 [M+H]⁺), 100% purity.

¹H NMR (400 MHz, MeOH-d₄) δ: 10.07 (s, 1H), 8.99 (d, J=5.7 Hz, 1H), 8.76 (d, J=8.4 Hz, 1H), 8.53 (d, J=1.6 Hz, 1H), 8.44 (d, J=5.6 Hz, 1H), 8.03 (dd, J=1.7, 8.5 Hz, 1H), 7.69 (d, J=1.7 Hz, 1H), 7.55-7.37 (m, 7H), 4.33 (s, 2H), 3.90 (t, J=6.7 Hz, 2H), 3.45-3.38 (m, 4H), 2.81 (t, J=6.7 Hz, 2H), 2.11 (quin, J=6.5 Hz, 2H) ppm.

### Example 47

### 5-((3-(3-(((2-chloro-[1,1′-biphenyl]-4-yl)methyl)amino)-N-methylpropanamido)propyl)amino)benzo[c][2,6]naphthyridine-8-carboxamide

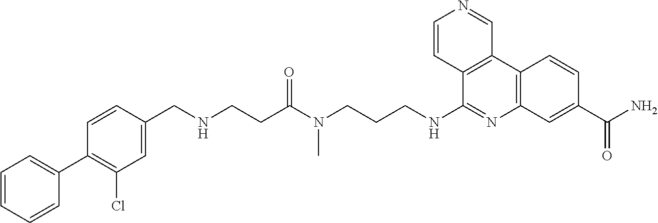 [SMILES: CN(CCCNc1nc2cc(C(N)=O)ccc2c2cnccc12)C(=O)CCNCc1ccc(-c2ccccc2)c(Cl)c1]

To a solution of compound 1.44 (110 mg, 0.16 mmol) in DCM (5 mL) was added TFA (1.54 g, 13.51 mmol) at 0° C. The reaction mixture was then warmed to 25° C. and stirred for 0.5 h. The reaction mixture was concentrated in vacuo and purified (PM126) to afford EXAMPLE 47 (59.2 mg, 50.9% yield, TFA salt) as a yellow solid.

LCMS (AM3): rt=0.750 min, (581.2 [M+H]⁺), 96.5% purity.

¹H NMR (400 MHz, MeOH-d₄) δ: 10.04 (s, 1H), 8.87 (d, J=5.6 Hz, 1H), 8.67 (d, J=8.4 Hz, 1H), 8.27 (d, 1H), 8.19 (s, 1H), 7.85 (d, J=1.2 Hz, 1H), 7.71 (s, 1H), 7.48-7.41 (m, 7H), 4.25-4.22 (m, 2H), 3.71-3.68 (m, 2H), 3.60-3.56 (m, 2H), 3.24-3.20 (m, 2H), 3.22-3.19 (m, 3H), 2.80-2.76 (m, 2H), 2.04-2.00 (m, 2H) ppm.

### Example 48

### 5-((2-(4-(((2-Chloro-[1,1′-biphenyl]-4-yl)methyl)amino)butanamido)ethyl)(methyl)amino)benzo[c][2,6]naphthyridine-8-carboxamide

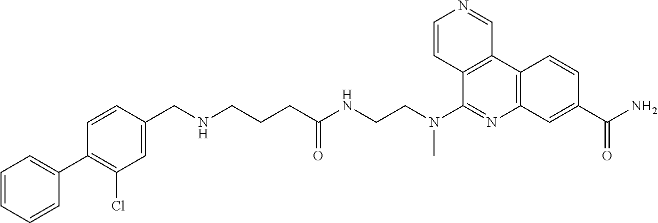 [SMILES: CN(CCNC(=O)CCCNCc1ccc(-c2ccccc2)c(Cl)c1)c1nc2cc(C(N)=O)ccc2c2cnccc12]

To a solution of compound 1.239 (150 mg, 220.19 μmol) in DCM (5 mL) was added TFA (27.01 mmol, 2 mL) at 20° C. The resulting mixture was stirred at 20° C. for 15 h. The mixture was concentrated in vacuo and the residue was purified (PM127) to afford EXAMPLE 48 (87.72 mg, 149.44 μmol, 67.9% yield) as an orange solid.

LCMS (AM3): rt=0.830 min, (581.4 [M+H]⁺), 99.1% purity.

¹H NMR (400 MHz, MeOH-d₄) δ: 10.12 (s, 1H), 8.90 (d, J=5.7 Hz, 1H), 8.76 (d, J=8.6 Hz, 1H), 8.58 (d, J=1.5 Hz, 1H), 8.37 (d, J=5.9 Hz, 1H), 8.05 (dd, J=1.8, 8.5 Hz, 1H), 7.68 (d, J=1.6 Hz, 1H), 7.51-7.36 (m, 7H), 4.26 (s, 2H), 3.92 (t, J=6.7 Hz, 2H), 3.68 (t, J=6.7 Hz, 2H), 3.51 (s, 3H), 3.16 (t, J=7.5 Hz, 2H), 2.39 (t, J=6.7 Hz, 2H), 2.02-1.95 (quin, 2H) ppm.

### Example 49

### 5-((3-(3-(((2-Chloro-[1,1′-biphenyl]-4-yl)methyl)amino)propanamido)-3-methylbutyl)amino)benzo[c][2,6]naphthyridine-8-carboxamide

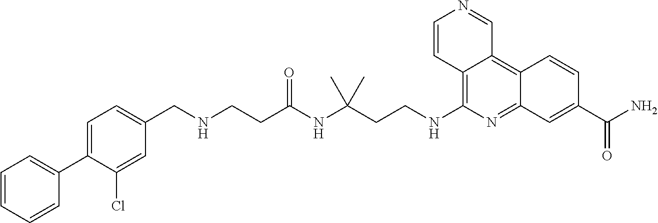 [SMILES: CC(C)(CCNc1nc2cc(C(N)=O)ccc2c2cnccc12)NC(=O)CCNCc1ccc(-c2ccccc2)c(Cl)c1]

To a solution of compound 1.95 (120 mg, 148.28 μmol) in DCM (5 mL) was added TFA (1.54 g, 13.51 mmol) at 25° C. The reaction mixture was stirred for 1 h. The mixture was concentrated in vacuo and purified (PM52) to afford EXAMPLE 49 (69.97 mg, 94.82 μmol, 63.9% yield, 96.1% purity, TFA salt) as a yellow solid.

LCMS (AM3): rt=0.771 min, (595.5 [M+H]⁺), 96.1% purity

¹H NMR (400 MHz, MeOH-d₄) δ: 10.09 (s, 1H), 8.99 (d, J=5.8 Hz, 1H), 8.80-8.78 (m, 2H), 8.33 (d, J=5.8 Hz, 1H), 8.08 (dd, J=1.6, 8.4 Hz, 1H), 7.70 (d, J=1.8 Hz, 1H), 7.53 (dd, J=1.8, 8.0 Hz, 1H), 7.45-7.33 (m, 6H), 4.41 (s, 2H), 3.87-3.78 (m, 2H), 3.57 (t, J=7.0 Hz, 2H), 2.89 (t, J=7.0 Hz, 2H), 2.48-2.40 (m, 2H), 1.42 (s, 6H) ppm.

### Example 50

### 5-((2-(4-(((2-Chloro-[1,1′-biphenyl]-4-yl)methyl)amino)-N-methylbutanamido)ethyl)amino)benzo[c][2,6]naphthyridine-8-carboxamide

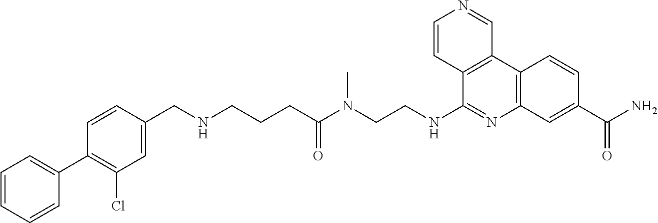 [SMILES: CN(CCNc1nc2cc(C(N)=O)ccc2c2cnccc12)C(=O)CCCNCc1ccc(-c2ccccc2)c(Cl)c1]

To a solution of compound 1.257 (160 mg, 234.18 μmol) in DCM (5 mL) was added TFA (1 mL, 15.03 mmol) at 15° C. The resulting mixture was stirred at 15° C. for 3 h. The solvent was removed in vacuo and the residue was purified (PM126) to afford EXAMPLE 50 (89.12 mg, 127.84 μmol, 54.6% yield, TFA salt) as a yellow solid.

LCMS (AM3): rt=0.774 min, (581.4 [M+H]⁺), 100% purity.

¹H NMR (400 MHz, DMSO-d₆) δ: 10.14 (d, J=3.5 Hz, 1H), 8.94 (dd, J=5.6, 11.4 Hz, 3H), 8.77 (dd, J=8.7, 10.6 Hz, 1H), 8.34-8.20 (m, 3H), 7.95-7.86 (m, 1H), 7.73 (dd, J=1.3, 4.8 Hz, 1H), 7.56-7.41 (m, 8H), 4.17 (t, J=5.2 Hz, 2H), 3.85-3.77 (m, 2H), 3.71-3.64 (m, 2H), 3.09-2.97 (m, 3H), 2.62-2.55 (m, 2H), 2.48-2.43 (m, 2H), 1.87-1.77 (m, 2H) ppm.

### Example 51

### 5-((2-(4-(2-(((2-Chloro-[1,1′-biphenyl]-4-yl)methyl)amino)ethyl)-1H-1,2,3-triazol-1-yl)ethyl)amino)benzo[c][2,6]naphthyridine-8-carboxamide

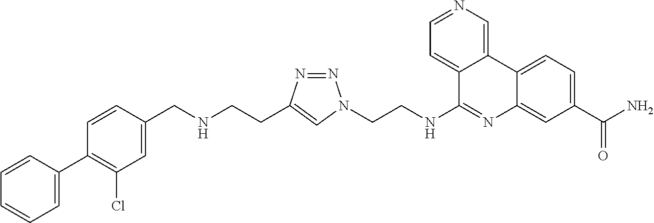 [SMILES: NC(=O)c1ccc2c(c1)nc(NCCn1cc(CCNCc3ccc(-c4ccccc4)c(Cl)c3)nn1)c1ccncc12]

A solution of compound 1.172 (230 mg, 339.64 μmol) in DCM (4 mL) and TFA (6.16 g, 54.02 mmol) was stirred at 35° C. for 3 h. The mixture was concentrated in vacuo and purified (PM44) to afford EXAMPLE 51 (101.08 mg, 146.26 μmol, 43.1% yield, TFA salt) as a yellow solid.

LCMS (AM3): rt=0.800 min, (577.2 [M+H]⁺), 100% purity.

¹H NMR (400 MHz, DMSO-d₆) δ: 10.00 (s, 1H), 8.85 (d, J=5.6 Hz, 1H), 8.65 (d, J=8.6 Hz, 1H), 8.22 (d, 1H), 8.19-8.17 (d, 1H), 7.97 (s, 1H), 7.89 (dd, J=8.4, 1.6 Hz, 1H), 7.64 (d, J=1.2 Hz, 1H), 7.48-7.38 (m, 7H), 4.92-4.85 (m, 2H), 4.27 (t, J=5.6 Hz, 2H), 4.22 (s, 2H), 3.28-3.25 (t, 2H), 3.07-3.04 (t, 2H) ppm.

### Example 52

### 5-((2-(5-(2-(((2-Chloro-[1,1′-biphenyl]-4-yl)methyl)amino)ethyl)-1,2,4-oxadiazol-3-yl)ethyl)amino)benzo[c][2,6]naphthyridine-8-carboxamide

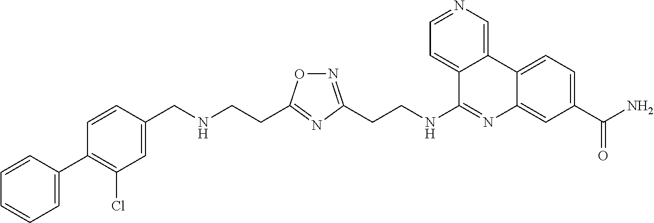 [SMILES: NC(=O)c1ccc2c(c1)nc(NCCc1noc(CCNCc3ccc(-c4ccccc4)c(Cl)c3)n1)c1ccncc12]

To a solution of compound 1.333 (100 mg, 147.45 μmol) in DCM (4 mL) was added TFA (2 mL, 27.01 mmol) at 35° C. The reaction mixture was stirred at 35° C. for 1 h. The mixture was concentrated in vacuo and the residue was purified (PM88) to afford EXAMPLE 52 (65.57 mg, 94.74 μmol, 64.25% yield, TFA salt) as a yellow solid.

LCMS (AM3): rt=0.787 min, (578.2 [M+H]⁺), 100% purity.

¹H NMR (400 MHz, MeOH-d₄) δ: 10.03 (s, 1H), 8.90 (d, J=6.0 Hz, 1H), 8.70 (d, J=8.4 Hz, 1H), 8.31 (d, J=1.6 Hz, 1H), 8.27 (d, J=5.6 Hz, 1H), 7.94 (dd, J=1.8, 8.4 Hz, 1H), 7.70 (d, J=1.2 Hz, 1H), 7.52-7.50 (m, 1H), 7.47-7.37 (m, 6H), 4.35 (s, 2H), 4.22 (t, J=6.6 Hz, 2H), 3.62 (t, J=7.2 Hz, 2H), 3.39 (t, J=7.2 Hz, 2H), 3.35-3.32 (m, 2H) ppm.

### Example 53

### 5-((3-(N-(2-(((2-chloro-[1,1′-biphenyl]-4-yl)methyl)amino)ethyl)sulfamoyl)propyl)amino)benzo[c][2,6]naphthyridine-8-carboxamide

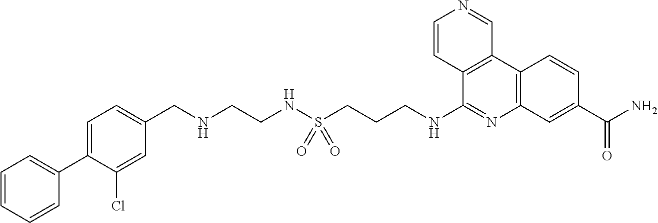 [SMILES: NC(=O)c1ccc2c(c1)nc(NCCCS(=O)(=O)NCCNCc1ccc(-c3ccccc3)c(Cl)c1)c1ccncc12]

To a solution of compound 1.380 (35 mg, 49.77 μmol) in DCM (2 mL) was added TFA (3.08 g, 27.01 mmol, 2 mL), and the resulting mixture was stirred at 15° C. for 1 h. The mixture was concentrated in vacuo and the residue was purified (PM128) to afford EXAMPLE 53 (6.24 mg, 8.53 μmol, 17% yield, TFA salt) as yellow solid.

LCMS (AM3): rt=0.748 min, (603.2 [M+H]⁺), 98.0% purity.

¹H NMR (400 MHz, MeOH-d₄) δ: 10.04 (s, 1H), 8.90 (d, J=6.0 Hz, 1H), 8.72 (d, J=8.4 Hz, 1H), 8.36 (d, J=1.6 Hz, 1H), 8.27 (d, J=5.6 Hz, 1H), 7.95 (dd, J=1.6, 8.4 Hz, 1H), 7.69 (d, J=1.2 Hz, 1H), 7.51-7.40 (m, 7H), 4.30 (s, 2H), 3.96 (t, J=7.2 Hz, 2H), 3.49 (t, J=6.0 Hz, 2H), 3.42-3.37 (m, 2H), 3.25 (t, J=6.0 Hz, 2H), 2.43-2.33 (quin, 2H) ppm.

### Example 54

### 5-((2-(2-(2-(((2-chloro-[1,1′-biphenyl]-4-yl)methyl)amino)ethyl)oxazol-5-yl)ethyl)amino)benzo[c][2,6]naphthyridine-8-carboxamide

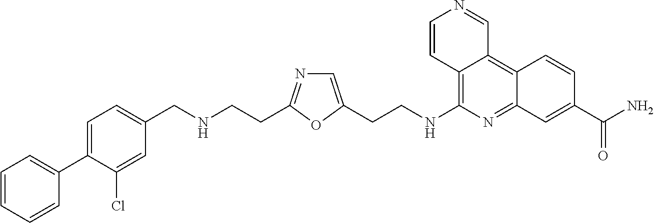 [SMILES: NC(=O)c1ccc2c(c1)nc(NCCc1cnc(CCNCc3ccc(-c4ccccc4)c(Cl)c3)o1)c1ccncc12]

To a mixture of compound 1.184 (100 mg, 147.67 μmol) in DCM (3 mL) was added TFA (4.62 g, 40.52 mmol) at 35° C. The reaction was stirred at 35° C. for 50 min. The reaction mixture was concentrated in vacuo and the residue was purified (PM44) to afford EXAMPLE 54 (87.25 mg, 126.25 μmol, 85.5% yield, TFA salt) as a yellow solid.

LCMS (AM3): rt=0.779 min, (577.2 [M+H]⁺), 100% purity.

¹H NMR (400 MHz, MeOH-d₄) δ 10.03 (s, 1H), 8.93 (d, J=5.6 Hz, 1H), 8.70 (d, J=8.4 Hz, 1H), 8.39 (s, 1H), 8.33 (d, J=5.6 Hz, 1H), 7.96 (dd, J=8.4, 1.2 Hz, 1H), 7.67 (s, 1H), 7.52-7.37 (m, 7H), 6.92 (s, 1H), 4.31 (s, 2H), 4.12 (t, J=6.8 Hz, 2H), 3.49 (t, J=7.2 Hz, 2H), 3.30-3.19 (m, 4H) ppm.

### Example 55

### 5-((2-(3-(2-(((2-Chloro-[1,1′-biphenyl]-4-yl)methyl)amino)ethyl)-1,2,4-oxadiazol-5-yl)ethyl)amino)benzo[c][2,6]naphthyridine-8-carboxamide

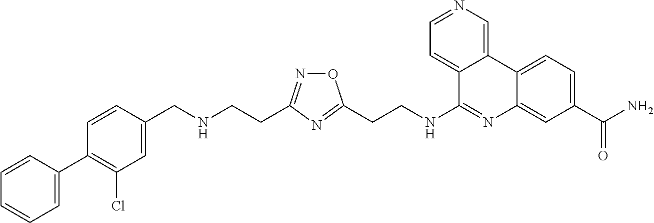 [SMILES: NC(=O)c1ccc2c(c1)nc(NCCc1nc(CCNCc3ccc(-c4ccccc4)c(Cl)c3)no1)c1ccncc12]

To a mixture of compound 1.192 (140 mg, 206.44 μmol) in DCM (4 mL) was added TFA (6.16 g, 54.02 mmol) at 35° C. The reaction mixture was stirred at 35° C. for 2 h. The mixture was concentrated in vacuo and the residue was purified (PM129) to afford EXAMPLE 55 (38.9 mg, 56.21 μmol, 27.2% yield, TFA salt) as a yellow solid.

LCMS (AM3): rt=0.793 min, (578.2 [M+H]⁺), 100% purity.

¹H NMR (400 MHz, MeOH-d₄) δ: 10.00 (s, 1H), 8.87 (d, J=5.6 Hz, 1H), 8.66 (d, J=8.4 Hz, 1H), 8.24 (s, 1H), 8.24-8.22 (d, 1H), 7.90 (dd, J=8.4, 1.6 Hz, 1H), 7.66 (d, J=1.6 Hz, 1H), 7.51-7.34 (m, 7H), 4.28 (s, 2H), 4.28-4.23 (t, 2H), 3.51-3.41 (m, 4H), 3.19-3.15 (t, 2H) ppm.

### Example 56

### 5-((2-(5-(2-(((2-Chloro-[1,1′-biphenyl]-4-yl)methyl)amino)ethyl)-4H-1,2,4-triazol-3-yl)ethyl)amino)benzo[c][2,6]naphthyridine-8-carboxamide

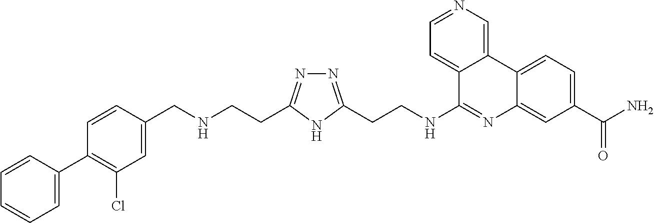 [SMILES: NC(=O)c1ccc2c(c1)nc(NCCc1nnc(CCNCc3ccc(-c4ccccc4)c(Cl)c3)[nH]1)c1ccncc12]

To a mixture of compound 1.195 (100 mg, 147.67 μmol) in DCM (4 mL) was added TFA (6.16 g, 54.02 mmol) at 35° C. The reaction mixture was stirred at 35° C. for 2 h. The mixture was concentrated in vacuo and the residue was purified (PM126) to afford EXAMPLE 56 (32.81 mg, 46.53 μmol, 31.5% yield, TFA salt) as a yellow solid.

LCMS (AM3): rt=0.775 min, (577.2 [M+H]⁺), 98.8% purity.

¹H NMR (400 MHz, MeOH-d₄) δ: 10.04 (s, 1H), 8.91 (d, J=5.6 Hz, 1H), 8.72 (d, J=8.4 Hz, 1H), 8.35 (d, J=1.6 Hz, 1H), 8.26 (d, J=5.6 Hz, 1H), 7.97 (dd, J=8.4, 1.6 Hz, 1H), 7.68 (d, J=1.6 Hz, 1H), 7.50-7.36 (m, 7H), 4.31 (s, 2H), 4.22 (t, J=6.4 Hz, 2H), 3.47 (t, J=7.6 Hz, 2H), 3.38-3.33 (m, 2H), 3.21-3.18 (t, 2H) ppm.

### Example 57

### 5-((2-(4-(2-(((2-Chloro-[1,1′-biphenyl]-4-yl)methyl)amino)ethyl)-1H-imidazol-1-yl)ethyl)amino)benzo[c][2,6]naphthyridine-8-carboxamide

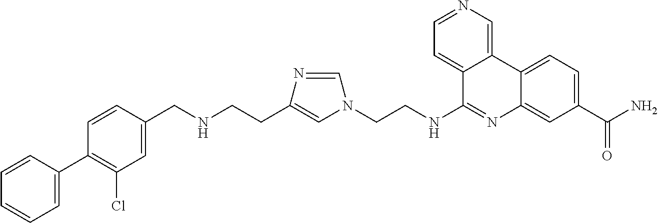 [SMILES: NC(=O)c1ccc2c(c1)nc(NCCn1cnc(CCNCc3ccc(-c4ccccc4)c(Cl)c3)c1)c1ccncc12]

A mixture of compound 1.354 (40 mg, 0.059 mmol) in DCM (10 mL) and TFA (5 mL, 67.53 mmol) was stirred at RT for 2 h. The mixture was concentrated in vacuo and purified (PM130) to give EXAMPLE 57 (10.12 mg, 14.47 μmol, 54.9% yield, TFA salt) as an off-white solid.

LCMS (AM3): rt=0.742 min, (576.2 [M+H]+), 98.7% purity.

¹H NMR (400 MHz, MeOH-d₄) δ: 10.00 (s, 1H), 8.86-8.85 (d, 2H), 8.67 (d, J=8.4 Hz, 1H), 8.13 (d, J=5.6 Hz, 1H), 8.05 (d, J=1.6 Hz, 1H), 7.87 (dd, J=2.0, 8.4 Hz, 1H), 7.70-7.69 (m, 2H), 7.54-7.41 (m, 7H), 4.57 (t, J=4.4 Hz, 2H), 4.29 (s, 2H), 4.23 (t, J=4.8 Hz, 2H), 3.32-3.30 (m, 2H), 3.06 (t, J=8.2 Hz, 2H) ppm.

### Example 58

### 5-((2-(2-(2-((3-Chloro-4-(trifluoromethoxy)benzyl)amino)ethyl) oxazol-5-yl)ethyl)amino)benzo[c][2,6]naphthyridine-8-carboxamide

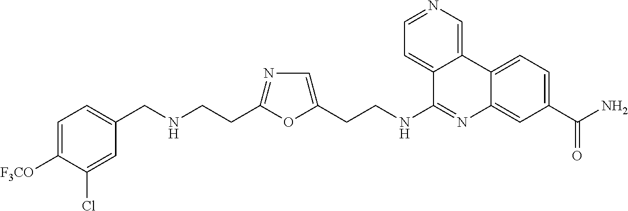 [SMILES: NC(=O)c1ccc2c(c1)nc(NCCc1cnc(CCNCc3ccc(OC(F)(F)F)c(Cl)c3)o1)c1ccncc12]

To a mixture of compound 1.251 (140 mg, 204.35 μmol) in DCM (3 mL) was added TFA (3.40 g, 29.86 mmol) at 35° C. The mixture was stirred at 35° C. for 2 h. The mixture was concentrated in vacuo and the residue was purified (PM69) to afford EXAMPLE 58 (49.88 mg, 71.36 μmol, 34.9% yield, TFA salt) as a yellow solid.

LCMS (AM3): rt=0.768 min, (585.2 [M+H]⁺), 100% purity.

¹H NMR (400 MHz, MeOH-d₄) δ: 10.05 (s, 1H), 8.91 (d, J=5.6 Hz, 1H), 8.71 (d, J=8.4 Hz, 1H), 8.34 (d, J=1.6 Hz, 1H), 8.28 (d, J=5.6 Hz, 1H), 7.94 (dd, J=8.4, 1.6 Hz, 1H), 7.76 (s, 1H), 7.55 (d, J=0.8 Hz, 2H), 6.89 (s, 1H), 4.29 (s, 2H), 4.10 (t, J=6.8 Hz, 2H), 3.46 (t, J=6.8 Hz, 2H), 3.25-3.18 (quin, 4H) ppm.

### Example 59

### 5-((2-(5-(2-((3-Chloro-4-(trifluoromethoxy)benzyl)amino)ethyl)-4H-1,2,4-triazol-3-yl)ethyl)amino)benzo[c][2,6]naphthyridine-8-carboxamide

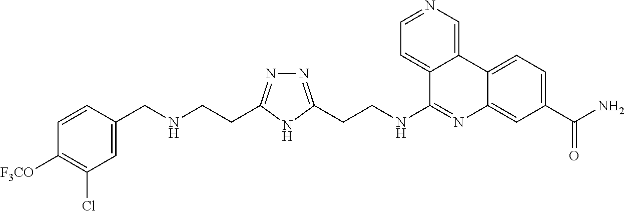 [SMILES: NC(=O)c1ccc2c(c1)nc(NCCc1nnc(CCNCc3ccc(OC(F)(F)F)c(Cl)c3)[nH]1)c1ccncc12]

To a mixture of compound 1.227 (50 mg, 72.98 μmol) in DCM (2 mL) was added TFA (3.08 g, 27.01 mmol) at 35° C. The reaction mixture was stirred at 35° C. for 2 h. The mixture was concentrated in vacuo and the residue was purified (PM130) to afford EXAMPLE 59 (22.31 mg, 31.28 μmol, 42.9% yield, TFA salt) as a yellow solid.

LCMS (AM3): rt=0.764 min, (585.2 [M+H]⁺), 98.5% purity.

¹H NMR (400 MHz, MeOH-d₄) δ: 10.06 (s, 1H), 8.92 (d, J=5.6 Hz, 1H), 8.74 (d, J=8.4 Hz, 1H), 8.36 (d, J=1.6 Hz, 1H), 8.27 (d, J=5.6 Hz, 1H), 7.98 (dd, J=8.4, 1.6 Hz, 1H), 7.78 (d, J=2.0 Hz, 1H), 7.59-7.52 (m, 2H), 4.31 (s, 2H), 4.22 (t, J=6.4 Hz, 2H), 3.47 (t, J=7.6 Hz, 2H), 3.36-3.33 (m, 2H), 3.18 (t, J=7.6 Hz, 2H) ppm.

### Example 60

### 5-((2-(2-(2-(((2-Chloro-[1,1′-biphenyl]-4-yl)methyl)amino)ethyl)-1H-imidazol-5-yl)ethyl)amino)benzo[c][2,6]naphthyridine-8-carboxamide

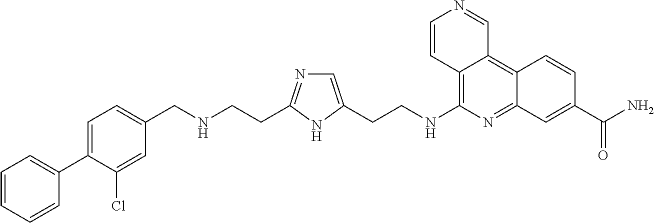 [SMILES: NC(=O)c1ccc2c(c1)nc(NCCc1cnc(CCNCc3ccc(-c4ccccc4)c(Cl)c3)[nH]1)c1ccncc12]

A mixture of compound 1.268 (30 mg, 0.044 mmol) and TFA (2 mL) in DCM (2 mL) was stirred at RT for 1 h. The reaction mixture was concentrated in vacuo and the residue was purified (PM115) to afford EXAMPLE 60 (3.14 mg, 5 μmol, 10.3% yield, TFA salt) as a yellow gum.

LCMS (AM3): rt=0.749 min, (576.2 [M+H]⁺), 100% purity

¹H NMR (400 MHz, MeOH-d₄) δ: 10.00 (s, 1H), 8.84 (d, J=5.6 Hz, 1H), 8.68 (d, J=8.4 Hz, 1H), 8.25 (d, J=2.0 Hz, 1H), 8.22 (d, J=6.0 Hz, 1H), 7.89 (dd, J=8.4 Hz, 2.0 Hz, 1H), 7.70 (d, J=2.0 Hz, 1H), 7.53-7.39 (m, 7H), 7.26 (s, 1H), 4.32 (s, 2H), 4.12 (t, J=6.4 Hz, 2H), 3.53-3.47 (m, 2H), 3.45-3.40 (m, 2H), 3.20 (t, J=6.4 Hz, 2H) ppm.

### Example 61

### 5-((2-(4-(2-((3-Chloro-4-(trifluoromethoxy)benzyl)amino)ethyl)-1H-imidazol-1-yl)ethyl)amino)benzo[c][2,6]naphthyridine-8-carboxamide

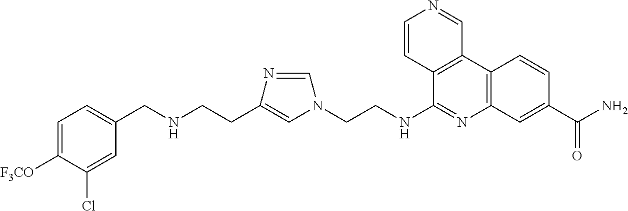 [SMILES: NC(=O)c1ccc2c(c1)nc(NCCn1cnc(CCNCc3ccc(OC(F)(F)F)c(Cl)c3)c1)c1ccncc12]

To a mixture of compound 1.233 (110 mg, 160.79 μmol) in DCM (3 mL) was added TFA (4.62 g, 40.52 mmol) at 20° C. The reaction mixture was stirred at 20° C. for 1 h. The reaction mixture was concentrated in vacuo and the residue was purified (PM127) to afford EXAMPLE 61 (55.27 mg, 79.18 μmol, 49.2% yield, TFA salt) as a yellow solid.

LCMS (AM3): rt=0.760 min, (584.2 [M+H]⁺), 100% purity.

¹H NMR (400 MHz, MeOH-d₄) δ: 10.00 (s, 1H), 8.89 (s, 1H), 8.88-8.84 (d, 1H), 8.65 (d, J=8.4 Hz, 1H), 8.15 (d, J=5.6 Hz, 1H), 8.06 (d, J=1.6 Hz, 1H), 7.86 (dd, J=8.4, 2.0 Hz, 1H), 7.78 (d, J=2.0 Hz, 1H), 7.67 (s, 1H), 7.58-7.52 (m, 2H), 4.56 (t, J=4.8 Hz, 2H), 4.27 (s, 2H), 4.21 (t, J=4.8 Hz, 2H), 3.31-3.28 (m, 2H), 3.03 (t, J=5.6 Hz, 2H) ppm.

### Example 62

### 5-((2-((4-((3-Chloro-4-(trifluoromethoxy)benzyl)amino)butyl)(methyl)amino)ethyl)amino)benzo[c][2,6]naphth yridine-8-carboxamide

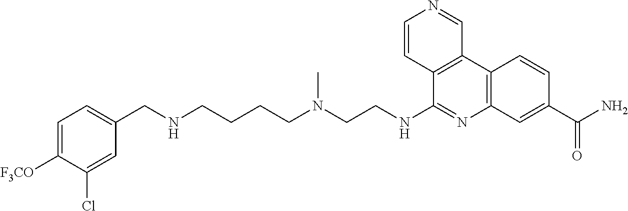 [SMILES: CN(CCCCNCc1ccc(OC(F)(F)F)c(Cl)c1)CCNc1nc2cc(C(N)=O)ccc2c2cnccc12]

To a solution of compound 1.163 (50 mg, 63.36 μmol) in DCM (5 mL) was added TFA (1 mL) at 20° C. The resulting mixture was stirred at 20° C. for 3 h. The mixture was concentrated in vacuo and purified (PM131) to afford EXAMPLE 62 (25.7 mg, 32.0 μmol, 50.5% yield, TFA salt) as a yellow gum.

LCMS (AM3): rt=0.745 min, (575.3 [M+H]⁺), 99.6% purity.

¹H NMR (400 MHz, MeOH-d₄) δ: 10.06 (s, 1H), 8.89 (d, J=5.6 Hz, 1H), 8.74 (d, J=8.5 Hz, 1H), 8.22 (d, 1H), 8.19-8.18 (d, 1H), 7.93 (dd, J=1.8, 8.5 Hz, 1H), 7.78 (d, J=1.8 Hz, 1H), 7.59-7.56 (m, 2H), 4.26 (s, 2H), 4.19-4.10 (m, 2H), 3.67-3.58 (m, 2H), 3.56-3.47 (m, 2H), 3.20-3.12 (m, 2H), 3.10 (s, 3H), 1.97-1.85 (m, 4H) ppm.

### Example 63

### 3-((3-Chloro-4-(trifluoromethoxy)benzyl)amino)-N-(3-((8-cyanobenzo[c][2,6]naphthyridin-5-yl)amino)propyl) propanamide

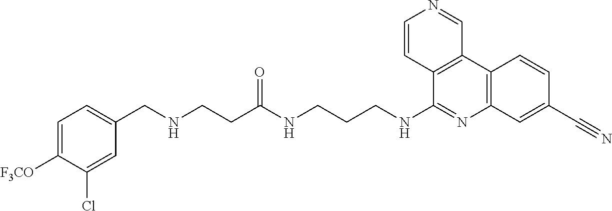 [SMILES: N#Cc1ccc2c(c1)nc(NCCCNC(=O)CCNCc1ccc(OC(F)(F)F)c(Cl)c1)c1ccncc12]

To a solution of compound 1.551 (400 mg, 608.75 μmol) in DCM (10 mL) was added TFA (15.40 g, 135.06 mmol) at 20° C. The reaction mixture was stirred at 20° C. for 2 h. The reaction mixture was concentrated in vacuo and purified (PM132) to afford EXAMPLE 63 (37.58 mg, 56.01 μmol, 9.2% yield, TFA salt) as a yellow solid.

LCMS (AM3): rt=0.810 min, (557.4 [M+H]⁺), 100% purity.

¹H NMR (400 MHz, MeOH-d₄) δ: 10.12 (s, 1H), 9.07 (d, J=5.6 Hz, 1H), 8.89 (d, J=8.4 Hz, 1H), 8.60 (d, J=5.6 Hz, 1H), 8.48 (d, J=1.2 Hz, 1H), 7.91 (dd, J=8.8, 1.6 Hz, 1H), 7.82 (d, J=2.0 Hz, 1H), 7.63-7.51 (m, 2H), 4.31 (s, 2H), 3.96 (t, J=6.8 Hz, 2H), 3.43 (t, J=6.8 Hz, 2H), 3.37 (t, J=6.8 Hz, 2H), 2.80 (t, J=6.8 Hz, 2H), 2.16-2.09 (quin, 2H) ppm.

### Example 64

### 5-((2-(4-(2-(((2-Chloro-[1,1′-biphenyl]-4-yl)methyl)amino)ethyl)-1H-1,2,3-triazol-1-yl)ethyl)amino)benzo[c][2,6]naphthyridine-8-carboxylic acid

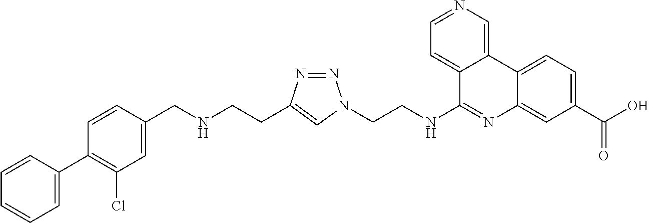 [SMILES: O=C(O)c1ccc2c(c1)nc(NCCn1cc(CCNCc3ccc(-c4ccccc4)c(Cl)c3)nn1)c1ccncc12]

To a solution of compound 1.552 (120 mg, 176.94 μmol) in DCM (15 mL) was added TFA (23.10 g, 202.60 mmol) at 20° C. The reaction mixture was stirred at 20° C. for 0.1 h. The reaction mixture was concentrated and purified (PM133) to afford EXAMPLE 64 (62.59 mg, 90.44 μmol, 51.11% yield, TFA salt) as a yellow solid.

LCMS (AM3): rt=0.812 min, (578.2 [M+H]⁺), 100% purity.

¹H NMR (400 MHz, DMSO-d₆) δ: 10.11 (s, 1H), 9.00-8.93 (br m, 2H), 8.90 (d, J=5.6 Hz, 1H), 8.78 (d, J=8.8 Hz, 1H), 8.25-8.16 (m, 3H), 8.05 (s, 1H), 7.88 (dd, J=8.0, 1.6 Hz, 1H), 7.73 (s, 1H), 7.53-7.40 (m, 7H), 4.76 (t, J=5.6 Hz, 2H), 4.23 (t, J=5.2 Hz, 2H), 4.05 (t, J=5.2 Hz, 2H), 3.24-3.16 (m, 2H), 2.98 (t, J=7.6 Hz, 2H) ppm.

### Example 65

### 5-((3-(4-((((2-Chloro-[1,1′-biphenyl]-4-yl)methyl)amino)methyl)-1H-1,2,3-triazol-1-yl)propyl)amino)benzo[c][2,6]naphthyridine-8-carboxamide

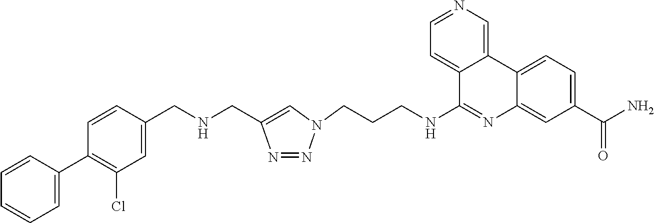 [SMILES: NC(=O)c1ccc2c(c1)nc(NCCCn1cc(CNCc3ccc(-c4ccccc4)c(Cl)c3)nn1)c1ccncc12]

To a solution of compound 1.557 (200 mg, 295.34 μmol) in DCM (10 mL) was added TFA (7.70 g, 67.53 mmol) at 20° C. The reaction mixture was stirred at 20° C. for 2 h. The reaction mixture was concentrated in vacuo and purified (PM94) to afford EXAMPLE 65 (34.04 mg, 49.25 μmol, 16.7% yield, TFA salt) as a yellow gum.

LCMS (AM3): rt=0.795 min, (577.5 [M+H]⁺), 97.6% purity.

¹H NMR (400 MHz, MeOH-d₄) δ: 10.01 (s, 1H), 8.92 (d, J=6.0 Hz, 1H), 8.69 (d, J=8.4 Hz, 1H), 8.40 (d, J=1.6 Hz, 1H), 8.33 (d, J=6.0 Hz, 1H), 8.26 (s, 1H), 7.96 (dd, J=8.4, 2.0 Hz, 1H), 7.67 (d, J=1.6 Hz, 1H), 7.52-7.35 (m, 7H), 4.69 (t, J=6.4 Hz, 2H), 4.44 (s, 2H), 4.35 (s, 2H), 3.89 (t, J=6.8 Hz, 2H), 2.52 (quint, J=6.8 Hz, 2H) ppm.

### Example 66

### 4-((3-(3-(((2-Chloro-[1,1′-biphenyl]-4-yl)methyl)amino)propanamido)propyl)amino)-3H-pyrazolo[3,4-c]quinoline-7-carboxylic acid

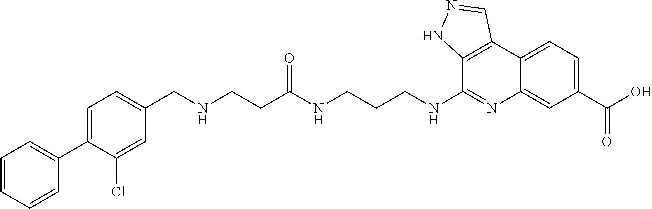 [SMILES: O=C(CCNCc1ccc(-c2ccccc2)c(Cl)c1)NCCCNc1nc2cc(C(=O)O)ccc2c2cn[nH]c12]

To a mixture of compound 1.777 (160 mg, 243.47 μmol) in DCM (3 mL) was added TFA (4.62 g, 40.52 mmol) at 30° C. The mixture was stirred at 30° C. for 1 h. The mixture was concentrated in vacuo and the residue was purified (PM134) to afford EXAMPLE 66 (125.84 mg, 187.52 μmol, 77.0% yield, TFA salt) as a white solid.

LCMS (AM3): rt=0.777 min, (557.2 [M+H]⁺), 100% purity.

¹H NMR (400 MHz, MeOH-d₄) δ: 8.85 (s, 1H), 8.61 (s, 1H), 8.19 (d, J=4.0 Hz, 1H), 8.11-8.08 (m, 1H), 7.67 (d, J=1.6 Hz, 1H), 7.50-7.37 (m, 7H), 4.30 (s, 2H), 3.83 (t, J=6.4 Hz, 2H), 3.44-3.40 (t, 2H), 3.40-3.36 (t, 2H) 2.75 (t, J=6.4 Hz, 2H), 2.09-2.03 (quin, 2H) ppm.

### Example 67

### 5-((2-((4-((3-Chloro-4-(trifluoromethoxy)benzyl)amino)butyl)amino)ethyl)amino)benzo[c][2,6]naphthyridine-8-carboxylic acid

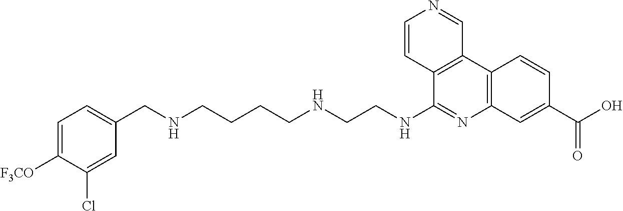 [SMILES: O=C(O)c1ccc2c(c1)nc(NCCNCCCCNCc1ccc(OC(F)(F)F)c(Cl)c1)c1ccncc12]

To a solution of compound 1.570 (25 mg, 37.76 μmol) in DCM (10 mL) was added TFA (15.40 g, 135.06 mmol) at 20° C. The reaction mixture was stirred at 20° C. for 2 h. The reaction mixture was concentrated in vacuo and purified (PM94) to afford EXAMPLE 67 (19.61 mg, 29.01 μmol, 76.83% yield, TFA salt) as a yellow solid.

LCMS (AM3): rt=0.769 min, (562.4 [M+H]⁺), 100% purity.

¹H NMR (400 MHz, MeOH-d₄) δ: 10.04 (s, 1H), 8.87 (d, J=5.6 Hz, 1H), 8.71 (d, J=8.4 Hz, 1H), 8.40 (d, J=1.6 Hz, 1H), 8.19 (d, J=5.6 Hz, 1H), 8.05 (dd, J=8.4, 1.6 Hz, 1H), 7.76 (s, 1H), 7.54 (s, 2H), 4.24 (s, 2H), 4.06 (t, J=5.2 Hz, 2H), 3.47 (t, J=5.2 Hz, 2H), 3.24 (t, J=7.2 Hz, 2H), 3.14 (t, J=7.2 Hz, 2H), 1.88-1.82 (m, 4H) ppm.

### Example 68

### 6-(3-(3-(((2-Chloro-[1,1′-biphenyl]-4-yl)methyl)amino)propanamido)propyl)-5-oxo-5,6-dihydrobenzo[c][2,6]naphthyridine-8-carboxylic acid

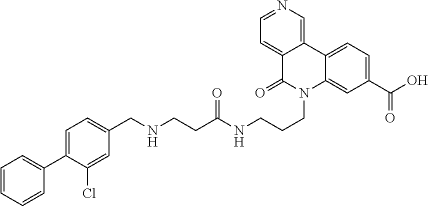 [SMILES: O=C(CCNCc1ccc(-c2ccccc2)c(Cl)c1)NCCCn1c(=O)c2ccncc2c2ccc(C(=O)O)cc21]

To a solution of compound 1.823 (100 mg, 149.44 μmol) in DCM (1 mL) was added TFA (17.04 mg, 149.44 μmol) at 20° C., and the mixture was stirred at 20° C. for 0.5 h. The reaction mixture was concentrated in vacuo and the residue was purified (PM135) to afford EXAMPLE 68 (62.3 mg, 109.48 μmol, 73.3% yield) as a white solid.

LCMS (AM3): rt=0.843 min, (569.2 [M+H]⁺), 99.2% purity.

¹H NMR (400 MHz, DMSO-d₆) δ: 9.93 (s, 1H), 8.83 (d, J=5.2 Hz, 1H), 8.69 (d, J=8.0 Hz, 1H), 8.39 (t, J=2.0 Hz 1H), 8.16-8.15 (d, 1H), 8.12 (s, 1H), 7.87 (d, J=8.0 Hz, 1H), 7.78 (s, 1H), 7.61 (d, J=5.2 Hz, 1H), 7.47-7.35 (m, 6H), 4.41 (t, J=7.2 Hz, 2H), 4.00 (s, 2H), 3.25-3.21 (m, 2H), 2.96 (t, J=6.4 Hz, 2H), 2.61 (t, J=4.8 Hz, 2H), 1.88-1.81 (m, 2H) ppm.

### Example 69

### 5-((2-((4-(((5-(Trifluoromethyl)-1H-indol-2-yl)methyl)amino)butyl)amino)ethyl)amino)benzo[c][2,6]naphthyridine-8-carboxylic acid

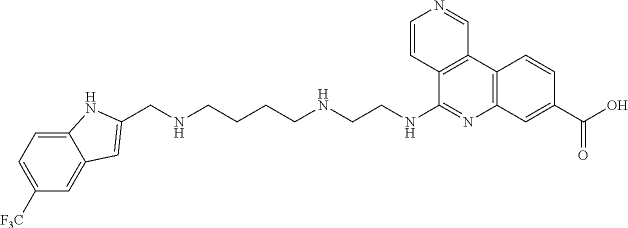 [SMILES: O=C(O)c1ccc2c(c1)nc(NCCNCCCCNCc1cc3cc(C(F)(F)F)ccc3[nH]1)c1ccncc12]

To a solution of compound 1.562 (30 mg, 46.10 μmol) in DCM (5 mL) was added TFA (7.70 g, 67.53 mmol) at 20° C. The reaction mixture was stirred at 20° C. for 2 h. The reaction mixture was concentrated in vacuo and purified (PM136) to afford EXAMPLE 69 (13.89 mg, 20.90 μmol, 45.3% yield, TFA salt) as a yellow solid.

LCMS (AM3): rt=0.778 min, (551.5 [M+H]⁺), 98.7% purity.

¹H NMR (400 MHz, MeOH-d₄) δ: 10.04 (s, 1H), 8.87 (d, J=5.6 Hz, 1H), 8.71 (d, J=8.4 Hz, 1H), 8.41 (d, J=1.6 Hz, 1H), 8.19 (d, J=5.6 Hz, 1H), 8.05 (dd, J=8.4, 1.6 Hz, 1H), 7.90 (s, 1H), 7.55 (d, J=8.4 Hz, 1H), 7.43 (dd, J=8.8, 1.6 Hz, 1H), 6.79 (s, 1H), 4.43 (s, 2H), 4.06 (t, J=5.2 Hz, 2H), 3.47 (t, J=5.6 Hz, 2H), 3.23 (t, J=7.6 Hz, 2H), 3.15 (t, J=6.8 Hz, 2H), 1.91-1.83 (m, 4H) ppm.

### Example 70

### 5-((2-(((3-(((2-Chloro-[1,1′-biphenyl]-4-yl)methyl)amino)cyclobutyl)methyl)amino)ethyl)amino)benzo[c][2,6]naphthyridine-8-carboxamide

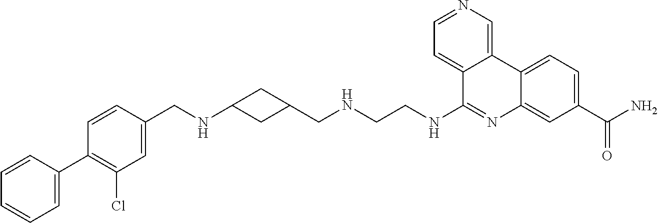 [SMILES: NC(=O)c1ccc2c(c1)nc(NCCNCC1CC(NCc3ccc(-c4ccccc4)c(Cl)c3)C1)c1ccncc12]

A mixture of compound 1.616 (260 mg, 0.117 mmol) and TFA (10 mL, 135.06 mmol) in DCM (10 mL) was stirred at RT for 1 h. The reaction mixture was concentrated in vacuo and purified (PM137) to afford EXAMPLE 70 (48.07 mg, 51.7% yield, TFA salt) as a yellow solid.

LCMS (AM3): rt=0.792 min, (565.2 [M+H]⁺), 100% purity.

¹H NMR (400 MHz, MeOH-d₄) δ: 10.01 (s, 1H), 8.86 (d, J=5.6 Hz, 1H), 8.69 (dd, J=8.4 Hz, 1.6 Hz, 1H), 8.28 (dd, J=8.0, 1.6 Hz, 1H), 8.19 (d, J=5.6 Hz, 1H), 7.93-7.89 (m, 1H), 7.65 (dd, J=4.0 Hz, 1.2 Hz, 1H), 7.48-7.38 (m, 7H), 4.15 (d, J=3.6 Hz, 2H), 4.06 (q, J=4.8 Hz, 2H), 4.01-3.74 (2×quin, 1H), 3.50-3.38 (m, 4H), 2.96-2.86 (m, 1H), 2.65-2.50 (m, 2H), 2.44-2.36 (m, 1H), 2.15-2.05 (m, 1H) ppm.

### Example 71

### 5-((2-(3-(2-(((2-Chloro-[1,1′-biphenyl]-4-yl)methyl)amino)ethyl)ureido)ethyl)amino)benzo[c][2,6]naphthyridine-8-carboxamide

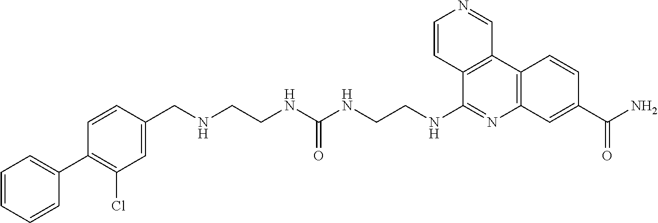 [SMILES: NC(=O)c1ccc2c(c1)nc(NCCNC(=O)NCCNCc1ccc(-c3ccccc3)c(Cl)c1)c1ccncc12]

To a solution of compound 1.338 (130 mg, 269.15 μmol) and TEA (269.15 μmol, 37.46 μL) in DMF (5 mL) was added CDI (43.64 mg, 269.15 μmol) at 20° C. under N₂. The mixture was stirred at 20° C. for 1 h and then a solution of Intermediate F (94.08 mg, 296.07 μmol) in DMF (0.5 mL) was added. The reaction mixture was stirred at 20° C. for 12 h. The reaction mixture was concentrated in vacuo and the residue purified (PM128) to afford EXAMPLE 71 (21.05 mg, 29.56 μmol, 10% yield, TFA salt) as a yellow solid.

LCMS (AM3): rt=0.715 min, (568.1 [M+H]⁺), 95.8% purity.

¹H NMR (400 MHz, MeOH-d₄, broad signals) δ: 10.04 (s, 1H), 8.96 (d, J=5.2 Hz, 1H), 8.73 (t, J=7.2 Hz, 1H), 8.59 (s, 1H), 8.30 (d, J=5.6 Hz, 1H), 8.02 (d, J=8.4 Hz, 1H), 7.66 (s, 1H), 7.50-7.25 (m, 7H), 4.36 (s, 2H), 3.89 (t, J=6.4 Hz, 2H), 3.64 (t, J=5.6 Hz, 2H), 3.56 (t, J=6.4 Hz, 2H), 3.35 (t, J=5.6 Hz, 2H) ppm.

### Example 72

### 5-((2-(3-(((2-Chloro-[1,1′-biphenyl]-4-yl)methyl)amino)propylsulfonamido)ethyl)amino)benzo[c][2,6]naphthyridine-8-carboxamide

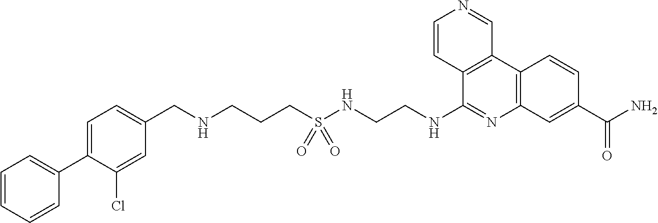 [SMILES: NC(=O)c1ccc2c(c1)nc(NCCNS(=O)(=O)CCCNCc1ccc(-c3ccccc3)c(Cl)c1)c1ccncc12]

To a mixture of compound 1.118 (90 mg, 0.213 mmol) and (2-chloro-[1,1′-biphenyl]-4-yl) methanamine (ref: Bioorganic and Medicinal Chemistry, 2017, 25 (13), 3471-3482, 90.00 mg, 0.393 mmol) in DMF (2 mL) was added NaI (31.98 mg, 0.213 mmol) at RT. The resulting mixture was then heated to 50° C. and stirred for 48 h. The reaction mixture was poured into H₂O and the aq. phase was extracted with EA (50 mL×2). The combined organic phases were washed (brine, 50 mL), dried (Na₂SO₄) and concentrated in vacuo. The residue was purified (PM127) to afford EXAMPLE 72 (25.23 mg, 0.0348 mmol, 16.3% yield, TFA salt) as a yellow solid.

LCMS (AM3): rt=0.783 min, (603.0 [M+H]⁺), 98.95% purity.

¹H NMR (400 MHz, DMSO-d₆) δ: 10.10 (s, 1H), 8.91-8.90 (d, 1H), 8.90-8.80 (br s, 2H), 8.74 (d, J=8.4 Hz, 1H), 8.26 (d, J=5.6 Hz, 1H), 8.17 (s, 2H), 7.85 (dd, J=8.4, 1.6 Hz, 1H), 7.71 (s, 1H), 7.51-7.40 (m, 9H), 4.23-4.17 (m, 2H), 3.86-3.81 (m, 2H), 3.40-3.34 (m, 2H), 3.23-3.18 (t, 2H), 3.15-3.08 (m, 2H), 2.10-2.01 (m, 2H) ppm.

### Example 73

### 5-((3-(2-(((2-Chloro-[1,1′-biphenyl]-4-yl)methyl)amino)ethylsulfonamido)propyl)amino)benzo[c][2,6]naphthyridine-8-carboxamide

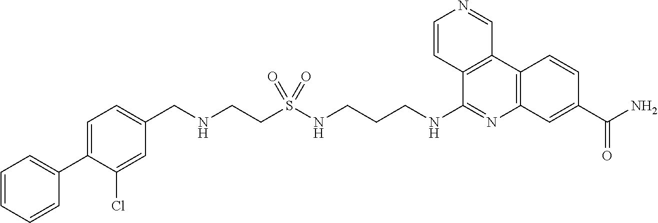 [SMILES: NC(=O)c1ccc2c(c1)nc(NCCCNS(=O)(=O)CCNCc1ccc(-c3ccccc3)c(Cl)c1)c1ccncc12]

A mixture of compound 1.335 (128.73 mg, 249.24 μmol), DIPEA (64.2 mg, 498.48 μmol) and 3-chloro-4-phenylbenzaldehyde (CAS 57592-44-6, 45 mg, 207.70 μmol) in MeOH (2 mL) was stirred for 2 h at 25° C., then NaBH(OAc)₃ (132.06 mg, 623.09 μmol) was added. The mixture was stirred at 25° C. for 12 h. The mixture was concentrated to give a residue which was purified (PM45) to give EXAMPLE 73 (17.66 mg, 24.63 μmol, 8.5% yield, TFA salt) as a yellow solid.

LCMS (AM3): rt=0.767 min, (603.2 [M+H]⁺), 100% purity.

¹H NMR (400 MHz, MeOH-d₄) δ: 10.03 (s, 1H), 8.92 (d, J=6.0 Hz, 1H), 8.73 (d, J=8.4 Hz, 1H), 8.39 (s, 1H), 8.32 (d, J=5.6 Hz, 1H), 7.96 (dd, J=1.6 Hz, 8.4 Hz, 1H), 7.65 (d, J=1.2 Hz, 1H), 7.47-7.35 (m, 7H), 4.29 (s, 2H), 3.92 (t, J=6.6 Hz, 2H), 3.51 (s, 4H), 3.35-3.32 (m, 2H), 2.12 (quin, J=6.4 Hz, 2H) ppm.

### Example 74

### N¹-(2-((8-(2H-tetrazol-5-yl)benzo[c][2,6]naphthyridin-5-yl)amino)ethyl)-N⁴-((2-chloro-[1,1′-biphenyl]-4-yl)methyl)butane-1,4-diamine

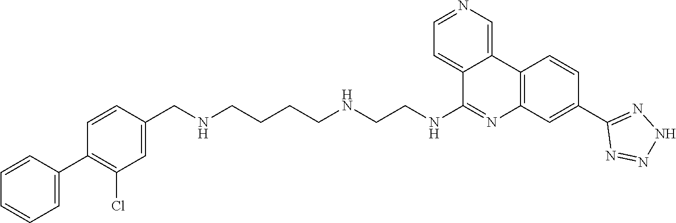 [SMILES: Clc1cc(CNCCCCNCCNc2nc3cc(-c4nn[nH]n4)ccc3c3cnccc23)ccc1-c1ccccc1]

A mixture of compound 1.385 (55 mg, 0.071 mmol) in a solution of HCl in 1,4-dioxane (4 M, 4 mL) was stirred at RT for 2 h. The reaction mixture was concentrated in vacuo and the residue was purified (PM138) to afford EXAMPLE 74 (10.37 mg, 22% yield, 2× HCl salt) as a yellow solid.

LCMS (AM1): rt=0.631 min, (578.2 [M+H]⁺), 97.8% purity.

¹H NMR (400 MHz, MeOH-d₄) δ: 10.22 (s, 1H), 8.99 (d, J=6.0 Hz, 1H), 8.87 (d, J=8.8 Hz, 1H), 8.65-8.62 (m, 2H), 8.14 (dd, J=8.4 Hz, 1.6 Hz, 1H), 7.71 (d, J=1.6 Hz, 1H), 7.54-7.51 (m, 1H), 7.48-7.39 (m, 6H), 4.28 (s, 2H), 4.19 (t, J=5.2 Hz, 2H), 3.56 (t, J=5.6 Hz, 2H), 3.29-3.25 (m, 2H), 3.20 (t, J=6.8 Hz, 2H), 1.96-1.90 (m, 4H) ppm.

### Example 75

### 5-((2-((4-((3-Chloro-4-(pyrimidin-5-yl)benzyl)amino)butyl)amino)ethyl)amino)benzo[c][2,6]naphthyridine-8-carboxamide

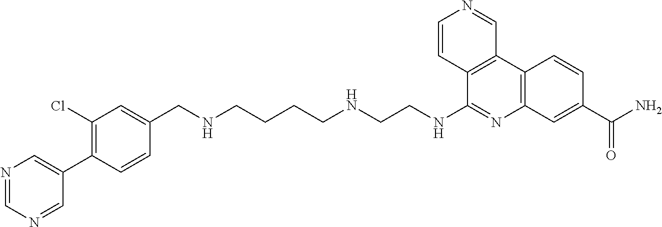 [SMILES: NC(=O)c1ccc2c(c1)nc(NCCNCCCCNCc1ccc(-c3cncnc3)c(Cl)c1)c1ccncc12]

A mixture of compound 1.221 (58 mg, 0.081 mmol, HCl salt) in a solution of HCl in 1,4-dioxane (4 M, 10 mL) was stirred at RT for 0.5 h. The reaction mixture was concentrated in vacuo and the residue was purified (PM139) to afford EXAMPLE 75 (19.98 mg, 39.1% yield, 2×HCl salt) as a yellow solid.

LCMS (AM3): rt=0.691 min, (555.1 [M+H]⁺), 100% purity.

¹H NMR (400 MHz, MeOH-d₄) δ: 10.28 (s, 1H), 9.26 (s, 1H), 9.06 (d, J=6.0 Hz, 1H), 8.97 (s, 2H), 8.90 (d, J=6.0 Hz, 1H), 8.79 (d, J=8.8 Hz, 1H), 8.55 (d, J=1.6 Hz, 1H), 8.03 (dd, J=8.4 Hz, 2.0 Hz, 1H), 7.84 (d, J=1.6 Hz, 1H), 7.68-7.58 (m, 2H), 4.32 (s, 2H), 4.27 (t, J=5.6 Hz, 2H), 3.60 (t, J=5.6 Hz, 2H), 3.29-3.24 (m, 2H), 3.19 (t, J=6.8 Hz, 2H), 1.96-1.86 (m, 4H) ppm.

### Example 76

### 5-((2-((4-(((6-Methyl-5-phenylpyridin-2-yl)methyl)amino)butyl)amino)ethyl)amino)benzo[c][2,6]naphthyridine-8-carboxamide

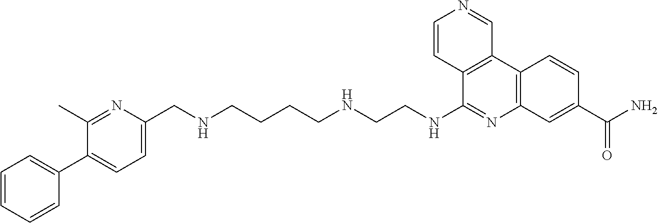 [SMILES: Cc1nc(CNCCCCNCCNc2nc3cc(C(N)=O)ccc3c3cnccc23)ccc1-c1ccccc1]

A mixture of compound 1.130 (160 mg, 0.214 mmol, TFA salt) in a solution of HCl in 1,4-dioxane (4 M, 10 mL) was stirred at RT for 18 h. The reaction mixture was concentrated in vacuo and purified (PM140) to afford EXAMPLE 76 (90.13 mg, 69.5% yield, HCl salt) as a yellow solid.

LCMS (AM3): rt=0.753 min, (534.3 [M+H]⁺), 100% purity.

¹H NMR (400 MHz, MeOH-d₄) δ: 10.27 (s, 1H), 9.05 (d, J=6.0 Hz, 1H), 8.86 (d, J=6.0 Hz, 1H), 8.78 (d, J=8.4 Hz, 1H), 8.54 (d, J=1.6 Hz, 1H), 8.07-8.01 (m, 2H), 7.74 (d, J=8.0 Hz, 1H), 7.56-7.46 (m, 3H), 7.43-7.41 (t, 2H), 4.56 (s, 2H), 4.26 (t, J=5.6 Hz, 2H), 3.60 (t, J=5.6 Hz, 2H), 3.30-3.26 (m, 4H), 2.64 (s, 3H), 2.10-1.90 (s, 4H) ppm.

### Example 77

### 5-((2-((4-((3-Chloro-4-(pyridin-2-yl)benzyl)amino)butyl)amino)ethyl)amino)benzo[c][2,6]naphthyridine-8-carboxamide

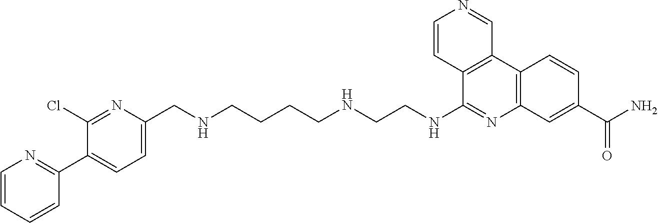 [SMILES: NC(=O)c1ccc2c(c1)nc(NCCNCCCCNCc1ccc(-c3ccccn3)c(Cl)n1)c1ccncc12]

A mixture of compound 1.164 (38 mg, 0.057 mmol, TFA salt) in a solution of HCl in 1,4-dioxane (4 M, 5 mL) was stirred at RT for 18 h. The reaction mixture was concentrated in vacuo and purified (PM141) to afford EXAMPLE 77 (4.91 mg, 13.4% yield, FA salt) as an off-white gum.

LCMS (AM3): rt=0.698 min, (554.2 [M+H]⁺), 100% purity.

¹H NMR (400 MHz, MeOH-d₄) δ: 9.97 (s, 1H), 8.83 (d, J=5.2 Hz, 1H), 8.69-8.61 (m, 2H), 8.50 (br s, 2H), 8.25 (d, J=1.2 Hz, 1H), 8.14 (d, J=5.6 Hz, 1H), 7.96-7.92 (t, 1H), 7.87 (dd, J=8.4 Hz, 1.6 Hz, 1H), 7.68-7.60 (m, 2H), 7.57-7.45 (m, 3H), 4.14 (s, 2H), 4.02 (t, J=4.4 Hz, 2H), 3.43 (t, J=4.4 Hz, 2H), 3.29-3.22 (m, 2H), 3.09-2.99 (m, 2H), 1.88-1.82 (m, 4H) ppm.

### Example 78

### 5-((2-((4-(((2-Chloro-2′-(2-hydroxyethoxy)-[1,1′-biphenyl]-4-yl)methyl)amino)butyl)amino)ethyl)amino)benzo[c][2,6]naphthyridine-8-carboxamide

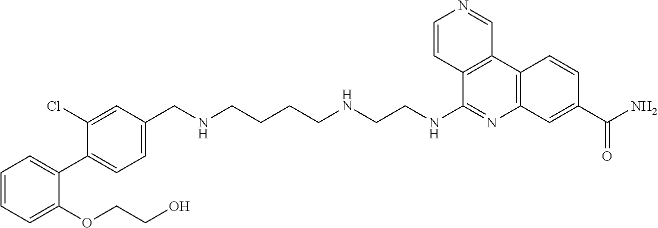 [SMILES: NC(=O)c1ccc2c(c1)nc(NCCNCCCCNCc1ccc(-c3ccccc3OCCO)c(Cl)c1)c1ccncc12]

To a mixture of compound 1.109 (38 mg, 0.043 mmol, FA salt) in 1,4-dioxane (0.5 mL) was added a solution of HCl in 1,4-dioxane (4 M, 0.5 mL) at 30° C. The reaction mixture was stirred at 30° C. for 1 h. The reaction mixture was concentrated in vacuo and purified (PM142) to afford EXAMPLE 78 (17.59 mg, 60% yield, HCl salt) as a yellow solid.

LCMS (AM3): rt=0.743 min, (613.3 [M+H]⁺), 100% purity.

¹H NMR (400 MHz, MeOH-d₄) δ: 10.27 (s, 1H), 9.06 (d, J=6.0 Hz, 1H), 8.90 (d, J=6.0 Hz, 1H), 8.77 (d, J=8.4 Hz, 1H), 8.54 (d, J=1.6 Hz, 1H), 8.02 (dd, J=8.4 Hz, 1.6 Hz, 1H), 7.67 (d, J=1.6 Hz, 1H), 7.49 (dd, J=8.0 Hz, 1.6 Hz, 1H), 7.42-7.36 (m, 2H), 7.14-7.09 (m, 2H), 7.05-7.01 (m, 1H), 4.30-4.25 (m, 4H), 4.04 (t, J=5.2 Hz, 2H), 3.70 (t, J=5.2 Hz, 2H), 3.60 (t, J=5.6 Hz, 2H), 3.27 (t, J=6.8 Hz, 2H), 3.21-3.15 (m, 2H), 1.96-1.87 (m, 4H) ppm.

### Example 79

### 5-(3-(3-((3-chloro-4-(trifluoromethoxy)benzyl)amino)propanamido)propyl)-4-oxo-4,5-dihydro-3H-pyrazolo[3,4-c]quinoline-7-carboxylic acid

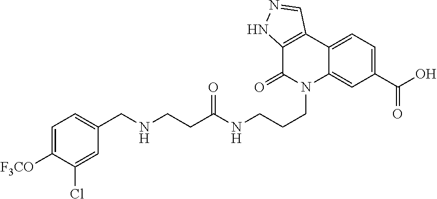 [SMILES: O=C(CCNCc1ccc(OC(F)(F)F)c(Cl)c1)NCCCn1c(=O)c2[nH]ncc2c2ccc(C(=O)O)cc21]

To a solution of compound 1.819 (60 mg, 79.98 μmol) in 1,4-dioxane (2 mL) was added a solution of HCl in 1,4-dioxane (2 mL, 4 M) at 20° C. The mixture was stirred at 20° C. for 0.5 h. The reaction mixture was concentrated in vacuo and the residue was purified (PM143) to afford EXAMPLE 79 (8.4 mg, 14.84 μmol, 18.6% yield) as a yellow oil.

LCMS (AM3): rt=0.784 min, (566.1 [M+H]⁺), 100% purity.

¹H NMR (400 MHz, DMSO-d₆) δ: 8.56 (s, 1H), 8.19 (t, J=5.2 Hz, 1H), 8.11-8.09 (m, 2H), 7.82 (d, J=8.4 Hz, 1H), 7.75 (s, 1H), 7.53-7.43 (m, 2H), 4.38 (t, J=7.6 Hz, 2H), 3.80 (s, 2H), 3.23-3.17 (m, 2H), 2.78 (t, J=6.0 Hz, 2H), 2.40 (t, J=6.4 Hz, 2H), 1.86-1.76 (m, 2H) ppm.

### Example 80

### 5-((4-(2-(((2-chloro-[1,1′-biphenyl]-4-yl)methyl)amino)ethoxy)butyl)amino)benzo[c][2,6]naphthyridine-8-carboxamide

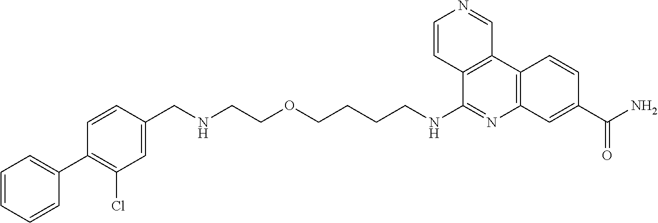 [SMILES: NC(=O)c1ccc2c(c1)nc(NCCCCOCCNCc1ccc(-c3ccccc3)c(Cl)c1)c1ccncc12]

A mixture of compound 1.24 (300 mg, 0.517 mmol) in a solution of NH₃ in MeOH (7 M, 10 mL) was heated to 80° C. and stirred for 16 h in an autoclave. The mixture was concentrated in vacuo to give a residue that was purified (PM122) to give EXAMPLE 80 (208 mg, 0.308 mmol, 59.7% yield, 99% purity, TFA salt) as a yellow solid.

LCMS (AM3): rt=0.754 min, (554.3 [M+H]⁺), 99.5% purity.

¹H NMR (400 MHz, DMSO-d₆) δ: 10.12 (s, 1H), 9.04 (br s, 2H), 8.94 (d, J=5.6 Hz, 1H), 8.77 (d, J=8.4 Hz, 1H), 8.40 (s, 1H), 8.33 (br s, 1H), 8.22 (s, 1H), 7.90 (d, J=7.8 Hz, 1H), 7.71 (d, J=1.6 Hz, 1H), 7.55 (s, 1H), 7.51-7.38 (m, 7H), 4.22 (s, 2H), 3.76-3.64 (m, 4H), 3.53 (t, J=6.4 Hz, 2H), 3.21-3.09 (m, 2H), 1.89-1.76 (m, 2H), 1.75-1.62 (m, 2H) ppm.

### Example 81

### 5-((2-(N-(3-(((2-chloro-[1,1′-biphenyl]-4-yl)methyl)amino)propyl)sulfamoyl)ethyl)amino)benzo[c][2,6]naphthyridine-8-carboxamide

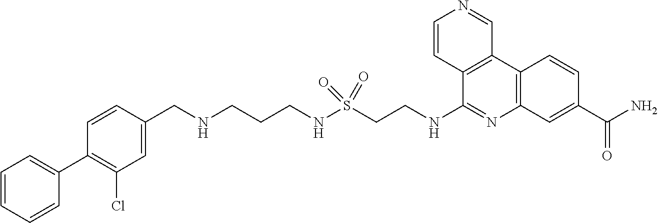 [SMILES: NC(=O)c1ccc2c(c1)nc(NCCS(=O)(=O)NCCCNCc1ccc(-c3ccccc3)c(Cl)c1)c1ccncc12]

A solution of compound 1.344 (100 mg, 136.58 μmol) in saturated NH₃ in MeOH (7 M, 10 mL) was stirred at 80° C. for 14 h in a 30 mL sealed tube. The mixture was concentrated in vacuo and the residue was purified (PM144) to afford EXAMPLE 81 (16.77 mg, 23.20 μmol, 17% yield, TFA salt) as a yellow solid.

LCMS (AM3): rt=0.774 min, (603.1 [M+H]⁺), 99.2% purity

¹H NMR (400 MHz, MeOH-d₄) δ: 10.00 (s, 1H), 8.85 (d, J=6.0 Hz, 1H), 8.67 (d, J=8.4 Hz, 1H), 8.29 (d, J=1.7 Hz, 1H), 8.17 (d, J=5.6 Hz, 1H), 7.90 (dd, J=1.8, 8.4 Hz, 1H), 7.67 (d, J=2.0 Hz, 1H), 7.50-7.37 (m, 7H), 4.28 (s, 2H), 4.17 (t, J=7.0 Hz, 2H), 3.62 (t, J=7.0 Hz, 2H), 3.35 (t, J=6.8 Hz, 2H), 3.26 (t, J=8.0 Hz, 2H), 2.09-2.02 (quin, 2H) ppm.

### Example 82

### 5-((2-(1-(2-(((2-Chloro-[1,1′-biphenyl]-4-yl)methyl)amino)ethyl)-1H-pyrazol-4-yl)ethyl)amino)benzo[c][2,6]naphthyridine-8-carboxamide

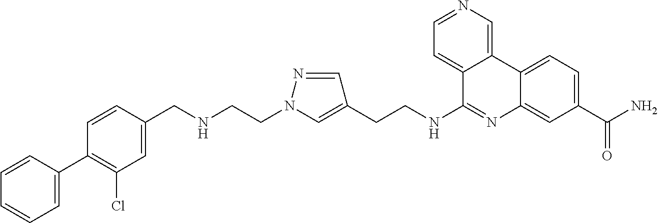 [SMILES: NC(=O)c1ccc2c(c1)nc(NCCc1cnn(CCNCc3ccc(-c4ccccc4)c(Cl)c3)c1)c1ccncc12]

A solution of compound 1.218 (80 mg, 113.46 μmol) in saturated NH₃ in MeOH (10 mL) was heated to 80° C. and stirred for 14 h in a 30 mL sealed tube. The reaction mixture was concentrated in vacuo and the residue was purified (PM45) to afford EXAMPLE 82 (15.24 mg, 22.08 μmol, 19.5% yield, TFA salt) as a yellow solid.

LCMS (AM3): rt=0.746 min, (576.0 [M+H]⁺), 100% purity.

¹H NMR (400 MHz, MeOH-d₄) δ: 10.05 (s, 1H), 8.94-8.92 (m, 1H), 8.75 (d, J=8.6 Hz, 1H), 8.40-8.31 (m, 2H), 8.00-7.96 (m, 1H), 7.69 (s, 1H), 7.62 (s, 1H), 7.60 (s, 1H), 7.47-7.37 (m, 7H), 4.48 (t, J=5.8 Hz, 2H), 4.22 (s, 2H), 4.01 (t, J=7.3 Hz, 2H), 3.51 (t, J=5.8 Hz, 2H), 3.08 (t, J=7.3 Hz, 2H) ppm.

### Example 83

### 5-((2-(1-(2-((3-Chloro-4-(trifluoromethoxy)benzyl)amino)ethyl)-1H-pyrazol-4-yl)ethyl)amino)benzo[c][2,6]naphthyridine-8-carboxamide

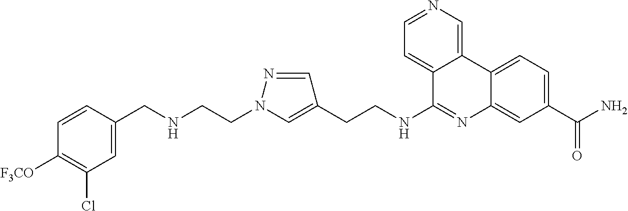 [SMILES: NC(=O)c1ccc2c(c1)nc(NCCc1cnn(CCNCc3ccc(OC(F)(F)F)c(Cl)c3)c1)c1ccncc12]

A solution of compound 1.348 (90 mg, 126.22 μmol) in saturated NH₃ in MeOH (5 mL, 7 M) was stirred at 80° C. for 48 h in a 30 mL sealed tube. The mixture was concentrated in vacuo and the residue was purified (PM146) to afford EXAMPLE 83 (32.39 mg, 55.07 μmol, 43.6% yield) as a yellow solid.

LCMS (AM3): rt=0.754 min, (584.2 [M+H]⁺), 99.3% purity.

¹H NMR (400 MHz, MeOH-d₄) δ: 10.06 (s, 1H), 8.95 (d, J=5.6 Hz, 1H), 8.75 (d, J=8.4 Hz, 1H), 8.41 (d, J=1.6 Hz, 1H), 8.36 (d, J=6.0 Hz, 1H), 8.00 (dd, J=1.6, 8.4 Hz, 1H), 7.73 (s, 1H), 7.68 (s, 1H), 7.58 (s, 1H), 7.52 (s, 2H), 4.46 (t, J=5.8 Hz, 2H), 4.24 (s, 2H), 4.01 (t, J=7.4 Hz, 2H), 3.50 (t, J=5.8 Hz, 2H), 3.07 (t, J=7.4 Hz, 2H) ppm.

### Example 84

### 5-((3-(3-((3-Chloro-4-(trifluoromethoxy)benzyl)amino)propoxy)propyl)amino)benzo[c][2,6]naphthyridine-8-carboxamide

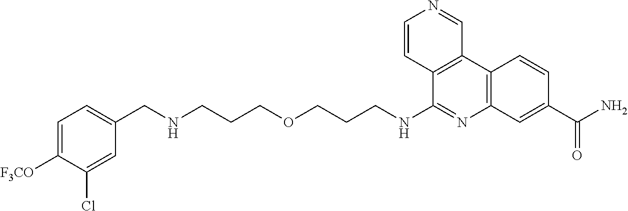 [SMILES: NC(=O)c1ccc2c(c1)nc(NCCCOCCCNCc1ccc(OC(F)(F)F)c(Cl)c1)c1ccncc12]

A solution of compound 1.541 (300 mg, 519.94 μmol) in a solution of NH₃ in MeOH (10 mL, 7 M) was stirred in a sealed tube at 90° C. for 12 h. The reaction mixture was concentrated in vacuo and purified (PM137) to afford EXAMPLE 84 (61.88 mg, 91.54 μmol, 17.6% yield, TFA salt) as a yellow solid.

LCMS (AM3): rt=0.760 min, (562.3 [M+H]⁺), 100% purity.

¹H NMR (400 MHz, MeOH-d₄) δ: 10.07 (s, 1H), 8.99 (d, J=6.0 Hz, 1H), 8.78 (d, J=8.4 Hz, 1H), 8.47 (d, J=1.6 Hz, 1H), 8.43 (d, J=6.0 Hz, 1H), 8.03 (dd, J=8.4, 1.6 Hz, 1H), 7.76 (d, J=2.0 Hz, 1H), 7.57-7.47 (m, 2H), 4.25 (s, 2H), 3.92 (t, J=7.2 Hz, 2H), 3.67 (t, J=6.0 Hz, 2H), 3.62 (t, J=6.0 Hz, 2H), 3.22 (t, J=7.6 Hz, 2H), 2.21-2.14 (quin, 2H), 2.08-1.99 (quin, 2H) ppm.

### Example 85

### 5-((4-(2-(((2-Chloro-[1,1′-biphenyl]-4-yl)methyl)amino)ethyl)phenethyl)amino)benzo[c][2,6]naphthyridine-8-carboxamide

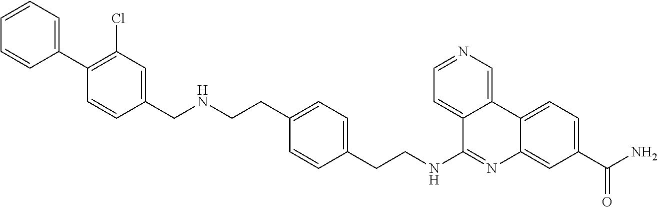 [SMILES: NC(=O)c1ccc2c(c1)nc(NCCc1ccc(CCNCc3ccc(-c4ccccc4)c(Cl)c3)cc1)c1ccncc12]

A solution of compound 1.628 (93 mg, 154.71 μmol) in a solution of NH₃ in MeOH (5 mL, 7 M) was stirred in a sealed tube at 90° C. for 24 h. The mixture was concentrated in vacuo to give the residue which was purified (PM146) to afford EXAMPLE 85 (57.24 mg, 96.97 μmol, 62.7% yield, TFA salt) as a yellow solid.

LCMS (AM3): rt=0.810 min, (586.4 [M+H]⁺), 99.4% purity.

¹H NMR (400 MHz, MeOH-d₄) δ: 10.04 (s, 1H), 8.95 (d, J=5.6 Hz, 1H), 8.73 (d, J=8.8 Hz, 1H), 8.35-8.33 (m, 2H), 7.99 (dd, J=8.4, 1.6 Hz, 1H), 7.66 (d, J=1.2 Hz, 1H), 7.49-7.37 (m, 7H), 7.36 (d, J=8.0 Hz, 2H), 7.22 (d, J=8.0 Hz, 2H), 4.24 (s, 2H), 4.08 (t, J=7.2 Hz, 2H), 3.22-3.16 (q, 4H), 2.96-2.92 (m, 2H) ppm.

### Example 86

### 5-((4-(2-((3-Chloro-4-(trifluoromethoxy)benzyl)amino)ethoxy)butyl)amino)benzo[c][2,6]naphthyridine-8-carboxamide

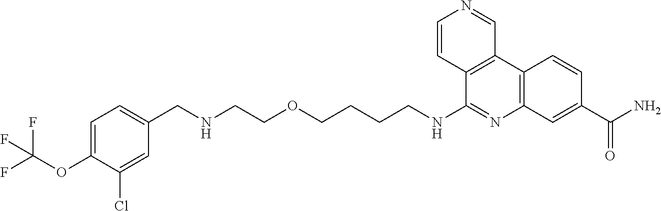 [SMILES: NC(=O)c1ccc2c(c1)nc(NCCCCOCCNCc1ccc(OC(F)(F)F)c(Cl)c1)c1ccncc12]

A solution of compound 1.431 (100 mg, 163.01 μmol) in NH₃ in MeOH (10 mL, 7 M) in a sealed tube was stirred at 90° C. for 24 h. The mixture was concentrated and purified (PM147) to afford EXAMPLE 86 (25.14 mg, 35.70 μmol, 21.9% yield, TFA salt) as a yellow oil.

LCMS (AM3): rt=0.748 min, (562.3 [M+H]⁺), 96.4% purity.

¹H NMR (400 MHz, MeOH-d₄) δ: 10.07 (s, 1H), 8.97 (d, J=5.6 Hz, 1H), 8.77 (d, J=8.4 Hz, 1H), 8.48 (d, J=1.6 Hz, 1H), 8.43 (d, J=5.6 Hz, 1H), 8.02 (dd, J=6.4, 2.0 Hz, 1H), 7.73 (d, J=2.0 Hz, 1H), 7.56-7.45 (m, 2H), 4.28 (s, 2H), 3.85 (t, J=7.2 Hz, 2H), 3.76 (t, J=4.8 Hz, 2H), 3.64 (t, J=6.0 Hz, 2H), 3.27 (t, J=4.8 Hz, 2H), 2.05-1.94 (quin, 2H), 1.87-1.80 (quin, 2H) ppm.

### Example 87

### 5-((3-(2-(((2-Chloro-[1,1′-biphenyl]-4-yl)methyl)amino)ethyl)phenethyl)amino)benzo[c][2,6]naphthyridine-8-carboxamide

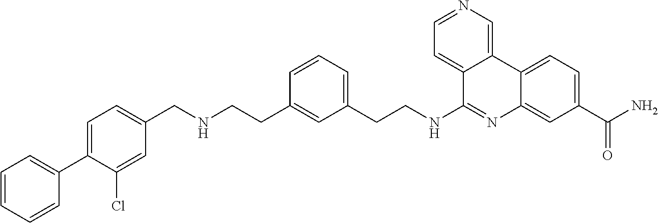 [SMILES: NC(=O)c1ccc2c(c1)nc(NCCc1cccc(CCNCc3ccc(-c4ccccc4)c(Cl)c3)c1)c1ccncc12]

A solution of compound 1.436 (140 mg, 232.89 μmol) in NH₃ in MeOH (10 mL, 7 M) in a sealed tube was stirred at 90° C. for 12 h. The mixture was concentrated in vacuo and purified (PM136) to afford EXAMPLE 87 (29.69 mg, 41.56 μmol, 17.8% yield, TFA salt) as a yellow solid.

LCMS (AM3): rt=0.730 min, (586.1 [M+H]⁺), 98.1% purity.

¹H NMR (400 MHz, MeOH-d₄) δ: 10.05 (s, 1H), 8.95 (d, J=5.6 Hz, 1H), 8.73 (d, J=8.8 Hz, 1H), 8.38-8.37 (d, 1H), 8.34-8.32 (d, 1H), 7.99 (dd, J=8.4, 1.6 Hz, 1H), 7.67 (d, J=1.6 Hz, 1H), 7.51-7.38 (m, 7H), 7.33 (s, 1H), 7.26 (d, J=4.2 Hz, 2H), 7.13-7.09 (m, 1H), 4.26 (s, 2H), 4.10 (t, J=7.2 Hz, 2H), 3.30-3.27 (m, 2H), 3.19 (t, J=7.2 Hz, 2H), 3.03-2.98 (t, J=7.2 Hz, 2H) ppm.

### Example 88

### 5-((3-((3-((3-Chloro-4-(trifluoromethoxy)benzyl)amino)propyl)amino)-3-oxopropyl)amino)benzo[c][2,6]naphthyridine-8-carboxamide

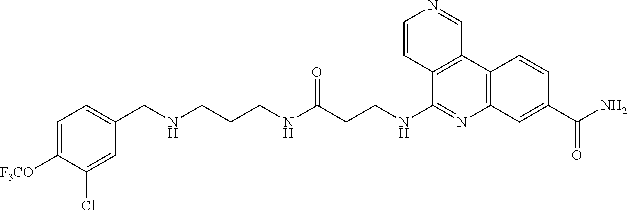 [SMILES: NC(=O)c1ccc2c(c1)nc(NCCC(=O)NCCCNCc1ccc(OC(F)(F)F)c(Cl)c1)c1ccncc12]

A solution of compound 1.544 (150 mg, 254.24 μmol) in a solution of NH₃ in MeOH (10 mL, 7 M) was stirred in a sealed tube at 90° C. for 12 h. The reaction mixture was concentrated in vacuo and purified (PM137) to afford EXAMPLE 88 (23.48 mg, 34.08 μmol, 13.4% yield, TFA salt) as a yellow solid.

LCMS (AM3): rt=0.737 min, (575.4 [M+H]⁺), 100% purity.

¹H NMR (400 MHz, MeOH-d₄) δ: 10.06 (s, 1H), 8.94 (d, J=5.6 Hz, 1H), 8.76 (d, J=8.8 Hz, 1H), 8.38 (d, J=1.6 Hz, 1H), 8.28 (d, J=5.6 Hz, 1H), 8.00 (dd, J=8.4, 1.6 Hz, 1H), 7.73 (s, 1H), 7.54-7.48 (m, 2H), 4.17 (s, 2H), 4.09 (t, J=6.0 Hz, 2H), 3.37 (t, J=6.8 Hz, 2H), 3.11 (t, J=7.2 Hz, 2H), 2.89 (t, J=6.0 Hz, 2H), 1.96-1.89 (quin, 2H) ppm.

### Example 89

### 5-((2-(4-((3-Chloro-4-(trifluoromethoxy)benzyl)amino)butanamido)ethyl)amino)benzo[c][2,6]naphthyridine-8-carboxamide

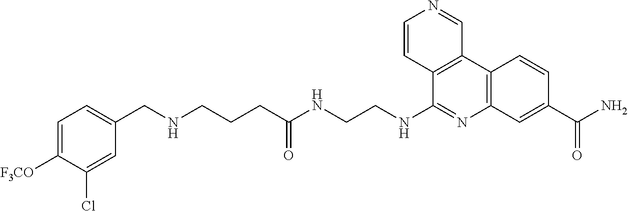 [SMILES: NC(=O)c1ccc2c(c1)nc(NCCNC(=O)CCCNCc1ccc(OC(F)(F)F)c(Cl)c1)c1ccncc12]

A mixture of compound 1.569 (145 mg, 245.77 μmol) in a solution of NH₃ in MeOH (10 mL, 7 M) was stirred in a sealed tube at 90° C. for 12 h. The reaction mixture was concentrated in vacuo and purified (PM137) to afford EXAMPLE 89 (16.17 mg, 23.47 μmol, 9.55% yield, TFA salt) as a yellow oil.

LCMS (AM3): rt=0.733 min, (575.1 [M+H]⁺), 100% purity.

¹H NMR (400 MHz, MeOH-d₄) δ: 10.07 (s, 1H), 8.95 (d, J=6.0 Hz, 1H), 8.77 (d, J=8.4 Hz, 1H), 8.59 (s, 1H), 8.28 (d, J=5.6 Hz, 1H), 8.03 (dd, J=8.4, 1.6 Hz, 1H), 7.78 (d, J=2.0 Hz, 1H), 7.60-7.46 (m, 2H), 4.29 (s, 2H), 3.91 (t, J=6.8 Hz, 2H), 3.62 (t, J=6.4 Hz, 2H), 3.25 (t, J=7.6 Hz, 2H), 2.52 (t, J=6.8 Hz, 2H), 2.07 (quint, J=7.6 Hz, 2H) ppm.

### Example 90

### 5-((2-(4-((((2-Chloro-[1,1′-biphenyl]-4-yl)methyl)amino)methyl)piperidin-1-yl)ethyl)amino)benzo[c][2,6]naphthyridine-8-carboxamide

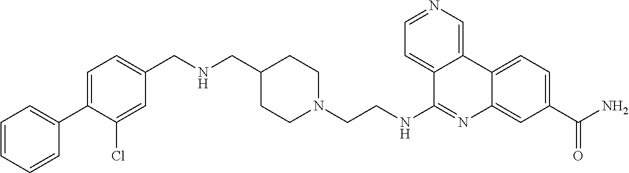 [SMILES: NC(=O)c1ccc2c(c1)nc(NCCN1CCC(CNCc3ccc(-c4ccccc4)c(Cl)c3)CC1)c1ccncc12]

Compound 1.442 (100 mg, 168.31 μmol) in a solution of NH₃ in MeOH (10 mL, 7 M) in a sealed tube was stirred at 90° C. for 12 h. The mixture was concentrated in vacuo and purified (PM94) to afford EXAMPLE 90 (52.37 mg, 74.04 μmol, 43.9% yield, TFA salt) as a yellow solid.

LCMS (AM3): rt=0.806 min, (579.2 [M+H]⁺), 98.8% purity.

¹H NMR (400 MHz, MeOH-d₄) δ: 10.05 (s, 1H), 8.88 (d, J=5.6 Hz, 1H), 8.72 (d, J=8.8 Hz, 1H), 8.32 (d, J=2.0 Hz, 1H), 8.20 (d, J=5.6 Hz, 1H), 7.93 (dd, J=8.4, 2.0 Hz, 1H), 7.72 (s, 1H), 7.54-7.51 (m, 1H), 7.48-7.37 (m, 6H), 4.30 (s, 2H), 4.14 (t, J=5.2 Hz, 2H), 3.98 (br d, J=12.0 Hz, 2H), 3.57 (t, J=5.2 Hz, 2H), 3.24-3.15 (m, 4H), 2.19-2.16 (m, 3H), 1.80-1.70 (m, 2H) ppm.

### Example 91

### 5-((4-((2-((3-Chloro-4-(trifluoromethoxy)benzyl)amino)ethyl)amino)-4-oxobutyl)amino)benzo[c][2,6]naphthyridine-8-carboxamide

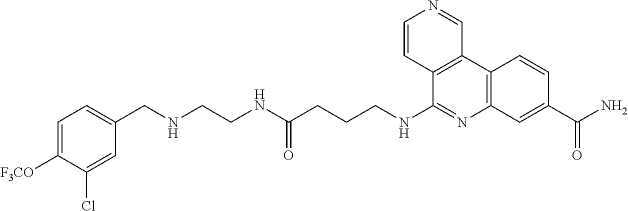 [SMILES: NC(=O)c1ccc2c(c1)nc(NCCCC(=O)NCCNCc1ccc(OC(F)(F)F)c(Cl)c1)c1ccncc12]

A solution of compound 1.659 (160 mg, 271.19 μmol) in a solution of NH₃ in MeOH (20 mL, 7 M) was stirred in a sealed tube at 85° C. for 12 h. The mixture was concentrated in vacuo and the residue was purified (PM148) to afford EXAMPLE 91 (27.9 mg, 17.9% yield) as a brown solid.

LCMS (AM3): rt=0.765 min, (575.3 [M+H]⁺), 99.5% purity.

¹H NMR (400 MHz, DMSO-d₆) δ: 10.05 (s, 1H), 8.85 (d, J=5.6 Hz, 1H), 8.69 (d, J=8.4 Hz, 1H), 8.27 (d, J=1.4 Hz, 1H), 8.17-8.13 (br s, 1H), 8.13-8.12 (d, 1H), 7.97-7.93 (t, 1H), 7.87-7.83 (t, 1H), 7.79 (dd, J=8.4, 2.0 Hz, 1H), 7.63 (d, J=1.6 Hz, 1H), 7.47-7.37 (m, 3H), 3.73 (s, 2H), 3.60 (q, J=5.2 Hz, 2H), 3.17 (q, J=6.0 Hz 2H), 2.57-2.50 (m, 2H), 2.25 (t, J=7.2 Hz, 2H), 2.00-1.90 (quin, 2H) ppm.

### Example 92

### 5-((2-(5-(((2-Chloro-[1,1′-biphenyl]-4-yl)methyl)amino)-2H-indazol-2-yl)ethyl)amino)benzo[c][2,6]naphthyridine-8-carboxamide

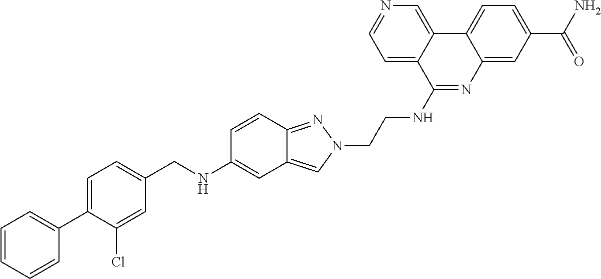 [SMILES: NC(=O)c1ccc2c(c1)nc(NCCn1cc3cc(NCc4ccc(-c5ccccc5)c(Cl)c4)ccc3n1)c1ccncc12]

A mixture of compound 1.515 (40 mg, 40.45 μmol) in a solution of NH₃ in MeOH (20 mL, 7 M) was heated in a sealed tube at 85° C. for 12 h. The mixture was concentrated in vacuo and the residue was purified (PM149) to afford EXAMPLE 92 (12.20 mg, 20.19 μmol, 49.9% yield) as a brown oil.

LCMS (AM3): rt=0.847 min, (598.2 [M+H]⁺), 99.6% purity.

¹H NMR (MeOH-d₄, 400 MHz) δ: 9.99 (s, 1H), 8.87 (d, J=5.6 Hz, 1H), 8.66 (d, J=8.4 Hz, 1H), 8.21-8.17 (m, 2H), 8.12 (s, 1H), 7.94 (dd, J=8.4, 1.6 Hz, 1H), 7.52-7.49 (m, 2H), 7.46-7.26 (m, 8H), 7.05 (d, J=8.2 Hz, 1H), 4.83-4.85 (m, 2H), 4.43 (s, 2H), 4.36 (t, J=5.6 Hz, 2H) ppm.

### Example 93

### 5-((2-(6-(((2-Chloro-[1,1′-biphenyl]-4-yl)methyl)amino)-1H-benzo[d]imidazol-2-yl)ethyl)amino)benzo[c][2,6]naphthyridine-8-carboxamide

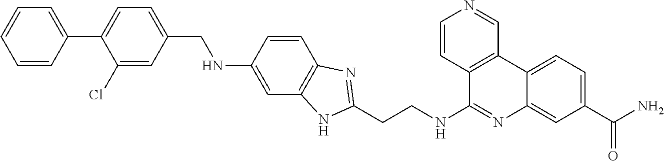 [SMILES: NC(=O)c1ccc2c(c1)nc(NCCc1nc3ccc(NCc4ccc(-c5ccccc5)c(Cl)c4)cc3[nH]1)c1ccncc12]

A mixture of compound 1.594 (60 mg, 97.86 μmol) in a solution of NH₃ in MeOH (10 mL, 7 M) was stirred in a sealed tube at 90° C. for 48 h. The mixture was concentrated in vacuo and the residue was purified (PM149) to afford EXAMPLE 93 (7.51 mg, 12.56 μmol, 12.8% yield) as a brown solid.

LCMS (AM3): rt=0.847 min, (598.2 [M+H]⁺), 100% purity.

¹H NMR (400 MHz, MeOH-d₄) δ: 9.96 (s, 1H), 8.81 (d, J=6.0 Hz, 1H), 8.57 (d, J=8.4 Hz, 1H), 8.10 (d, J=5.6 Hz, 1H), 7.96 (d, J=1.6 Hz, 1H), 7.81 (dd, J=8.4, 1.6 Hz, 1H), 7.47-7.27 (m, 9H), 6.84 (dd, J=8.8, 2.0 Hz, 1H), 6.48 (d, J=2.0 Hz, 1H), 4.38 (s, 2H), 4.22 (t, J=6.0 Hz, 2H), 3.50 (t, J=6.0 Hz, 2H) ppm.

### Example 94

### 5-((2-((3-(((3-Chloro-4-(trifluoromethoxy)benzyl)amino)methyl)cyclobutyl)amino)ethyl)amino)benzo[c][2,6]na phthyridine-8-carboxamide

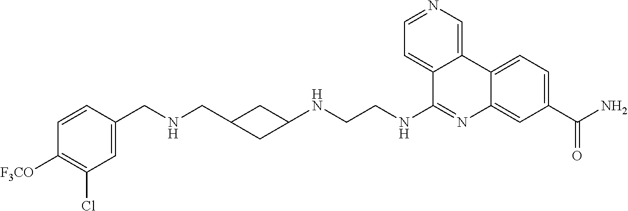 [SMILES: NC(=O)c1ccc2c(c1)nc(NCCNC1CC(CNCc3ccc(OC(F)(F)F)c(Cl)c3)C1)c1ccncc12]

A mixture of compound 1.597 (100 mg, 170.06 μmol) in a solution of NH₃ in MeOH (10 mL, 7 M) was stirred at 80° C. for 16 h. The mixture was concentrated in vacuo and the residue was purified (PM150) to afford EXAMPLE 94 (58.57 mg, 73.12 μmol, 42.9% yield) as a yellow solid.

LCMS (AM3): rt=0.786 min, (573.2 [M+H]⁺), 100% purity.

¹H NMR (400 MHz, MeOH-d₄) δ: 10.03 (s, 1H), 8.87 (d, J=5.6 Hz, 1H), 8.70 (dd, J=8.4, 2.0 Hz, 1H), 8.31 (t, J=1.6 Hz, 1H), 8.20 (d, J=5.6 Hz, 1H), 7.92 (dt, J=8.4, 2.0 Hz, 1H), 7.78-7.76 (m, 1H), 7.56-7.54 (m, 2H), 4.26-4.22 (m, 2H), 4.08-4.18 (m, 1H), 4.06-4.02 (q, 2H), 3.94-3.86 (m, 1H), 3.38 (t, J=5.2 Hz, 2H), 3.19 (d, J=6.8 Hz, 1H), 2.69-2.36 (m, 4H), 2.17-2.09 (m, 1H) ppm.

### Example 95

### 5-((2-(3-(2-((3-Chloro-4-(trifluoromethoxy)benzyl)amino)ethyl)pyrrolidin-1-yl)ethyl)amino)benzo[c][2,6]naphthyridine-8-carboxamide

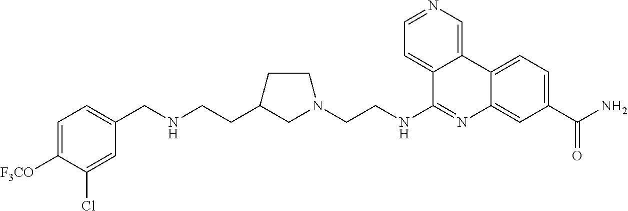 [SMILES: NC(=O)c1ccc2c(c1)nc(NCCN1CCC(CCNCc3ccc(OC(F)(F)F)c(Cl)c3)C1)c1ccncc12]

Compound 1.451 (75 mg, 107.44 μmol) in a solution of NH₃ in MeOH (10 mL, 7 M) in a sealed tube was stirred at 90° C. for 12 h. The mixture was concentrated in vacuo and the residue was purified (PM151) to afford EXAMPLE 95 (22.55 mg, 34.87 μmol, 32.4% yield, FA salt) as a yellow solid.

LCMS (AM3): rt=0.770 min, (587.3 [M+H]⁺), 97.9% purity.

¹H NMR (400 MHz, MeOH-d₄) δ: 9.97 (s, 1H), 8.83 (d, J=5.6 Hz, 1H), 8.66 (d, J=8.4 Hz, 1H), 8.40 (br s, 1H), 8.21 (d, J=1.6 Hz, 1H), 8.12 (d, J=5.6 Hz, 1H), 7.88 (dd, J=8.4, 1.6 Hz, 1H), 7.73 (s, 1H), 7.53-7.48 (m, 2H), 4.14 (s, 2H), 4.06 (t, J=5.0 Hz, 2H), 3.87 (t, J=9.2 Hz, 1H), 3.59 (t, J=5.2 Hz, 4H), 3.23 (t, J=9.2 Hz, 1H), 3.10-3.00 (m, 2H), 2.59-2.51 (m, 1H), 2.38-2.30 (m, 1H), 1.96-1.76 (m, 3H) ppm.

### Example 96

### 5-((2-(3-(N-(3-chlorobenzyl)sulfamoyl)propoxy)ethyl)amino)benzo[c][2,6]naphthyridine-8-carboxamide

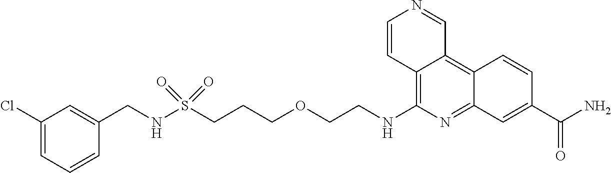 [SMILES: NC(=O)c1ccc2c(c1)nc(NCCOCCCS(=O)(=O)NCc1cccc(Cl)c1)c1ccncc12]

Compound 1.458 (300 mg, 552.45 μmol) in a solution of NH₃ in MeOH (20 mL, 7 M) in a sealed tube was stirred at 90° C. for 12 h. The mixture was concentrated in vacuo and purified (PM152) to afford EXAMPLE 96 (104.07 mg, 196.31 μmol, 35.5% yield) as a yellow solid.

LCMS (AM3): rt=0.787 min, (528.1 [M+H]⁺), 99.6% purity.

¹H NMR (400 MHz, MeOH-d₄) δ: 9.87 (s, 1H), 8.74 (d, J=5.6 Hz, 1H), 8.55 (d, J=8.4 Hz, 1H), 8.19 (d, J=1.6 Hz, 1H), 8.10 (d, J=5.6 Hz, 1H), 7.80 (dd, J=8.4, 2.0 Hz, 1H), 7.27-7.14 (m, 4H), 4.06 (s, 2H), 3.90 (t, J=5.2 Hz, 2H), 3.80 (t, J=5.6 Hz, 2H), 3.62 (t, J=6.0 Hz, 2H), 3.07 (t, J=5.6 Hz, 2H), 2.04-1.98 (m, 2H) ppm.

### Example 97

### (S)-5-((2-((4-((3-Chlorobenzyl)amino)pentyl)oxy)ethyl)amino)benzo[c][2,6]naphthyridine-8-carboxamide

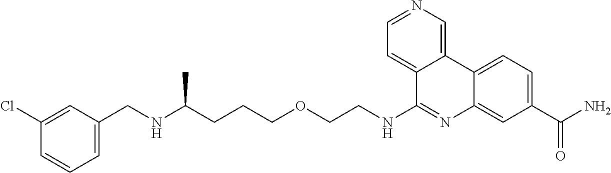 [SMILES: C[C@@H](CCCOCCNc1nc2cc(C(N)=O)ccc2c2cnccc12)NCc1cccc(Cl)c1]

A solution of compound 1.641 (100 mg, 161.02 μmol, TFA salt) in a solution of NH₃ in MeOH (15 mL, 7M) was stirred in a sealed tube at 90° C. for 10 h. The reaction mixture was concentrated in vacuo and purified (PM153) to afford EXAMPLE 97 (18.07 mg, 33.59 μmol, 20.7% yield, FA salt) as an off-white solid.

LCMS (AM3): rt=0.713 min, (492.3 [M+H]⁺), 100% purity.

¹H NMR (400 MHz, MeOH-d₄) δ: 9.91 (s, 1H), 8.77 (d, J=5.6 Hz, 1H), 8.59 (d, J=8.4 Hz, 1H), 8.50 (br s, 1H), 8.21 (d, J=2.0 Hz, 1H), 8.12 (d, J=5.6 Hz, 1H), 7.82 (dd, J=8.4, 2.0 Hz, 1H), 7.40-7.32 (m, 3H), 7.28-7.25 (m, 1H), 4.01 (d, J=2.4 Hz, 2H), 3.93 (t, J=5.6 Hz, 2H), 3.83 (t, J=5.6 Hz, 2H), 3.63 (t, J=4.8 Hz, 2H), 3.25-3.18 (m, 1H), 1.93-1.86 (m, 1H), 1.78-1.68 (m, 1H), 1.67-1.56 (m, 2H), 1.28 (d, J=6.4 Hz, 3H) ppm.

### Example 98

### 5-((4-(4-(((2-Chloro-[1,1′-biphenyl]-4-yl)methyl)amino)-1H-1,2,3-triazol-1-yl)butyl)amino)benzo[c][2,6]naphthyridine-8-carboxamide

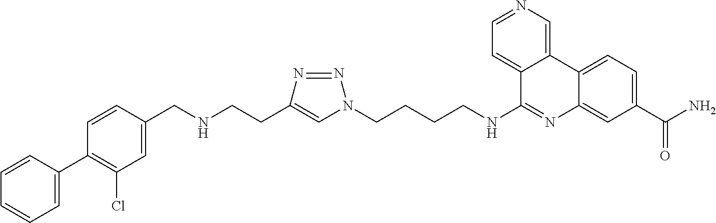 [SMILES: NC(=O)c1ccc2c(c1)nc(NCCCCn1cc(CCNCc3ccc(-c4ccccc4)c(Cl)c3)nn1)c1ccncc12]

A mixture of compound 1.427 (0.25 g, 305 mmol, 2×TFA salt) in NH₃ in MeOH (10 mL, 7 M) was stirred in a sealed tube at 90° C. for 16 h. The reaction mixture was concentrated in vacuo and the residue was purified (PM154) to afford EXAMPLE 98 (65.95 mg, 37.5% yield) as a yellow solid.

LCMS (AM3): rt=0.859 min, (577.3 [M+H]⁺), 100% purity.

¹H NMR (400 MHz, DMSO-d₆) δ: 10.05 (s, 1H), 8.84 (d, J=5.6 Hz, 1H), 8.69 (d, J=8.4 Hz, 1H), 8.25 (d, J=5.6 Hz, 1H), 8.18-8.15 (m, 2H), 7.91 (t, J=5.2 Hz, 1H), 7.80 (dd, J=8.4 Hz, 1.6 Hz, 1H), 7.50 (s, 1H), 7.45-7.31 (m, 7H), 7.30-7.28 (m, 1H), 7.23 (s, 1H), 6.01 (t, J=6.4 Hz, 1H), 4.28 (t, J=6.8 Hz, 2H), 4.23 (d, J=6.4 Hz, 2H), 3.65-3.61 (q, 2H), 1.94-1.87 (quin, 2H), 1.71-1.64 (quin, 2H) ppm.

### Example 99

### 5-((3-(3-((3-Chlorobenzyl)amino)propanamido)propyl)amino)benzo[c][2,6]naphthyridine-8-carboxamide

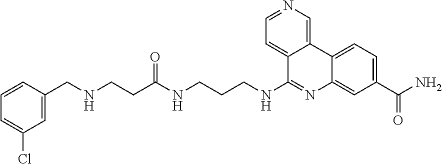 [SMILES: NC(=O)c1ccc2c(c1)nc(NCCCNC(=O)CCNCc1cccc(Cl)c1)c1ccncc12]

A solution of 3-chlorobenzaldehyde (23.02 mg, 163.75 μmol) and Intermediate C (60 mg, 163.75 μmol) in MeOH (1 mL) was stirred at 25° C. for 2 h, then NaBH₃CN (102.90 mg, 1.64 mmol) was added. The mixture was stirred at 25° C. for 12 h. The reaction mixture was concentrated in vacuo and purified (PM155) to afford EXAMPLE 99 (23.69 mg, 39.16 μmol, 23.9% yield, TFA salt) as a yellow solid.

LCMS (AM3): rt=0.658 min, (491.0 [M+H]⁺), 100% purity.

¹H NMR (400 MHz, MeOH-d₄) δ: 10.04 (s, 1H), 8.96 (d, J=5.7 Hz, 1H), 8.73 (d, J=8.4 Hz, 1H), 8.48 (d, J=1.6 Hz, 1H), 8.40 (d, J=5.7 Hz, 1H), 8.00 (dd, J=1.7, 8.4 Hz, 1H), 7.55 (s, 1H), 7.48-7.42 (m, 3H), 4.26 (s, 2H), 3.87 (t, J=6.6 Hz, 2H), 3.41 (t, J=6.5 Hz, 2H), 3.35 (t, J=6.7 Hz, 2H), 2.77 (t, J=6.7 Hz, 2H), 2.11-2.04 (quintet, J=6.6 Hz, 2H) ppm.

The following Examples in Table 4 were made with non-critical changes or substitutions to the exemplified procedure in EXAMPLE 99, that would be understood by one skilled in the art using Intermediate C and compounds of formula (Ic).

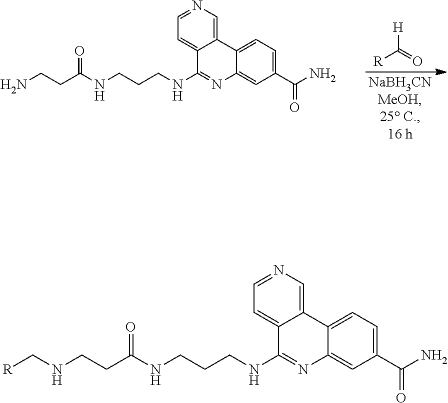

TABLE 4

| Example | Chemical IUPAC | | |
|---|---|---|---|
| No. | name | Compound (Ic) | Analytical |
| EXAMPLE 100 | 5-((3-(3-((3- Chloro-4- (trifluoromethoxy) benzyl)amino)pro- panamido)propyl) amino)benzo[c][2,6] naphthyridine-8- | 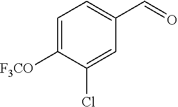 [SMILES: O=Cc1ccc(OC(F)(F)F)c(Cl)c1] | ¹H NMR (400 MHz, MeOH-d₄) δ: 10.03 (s, 1H), 8.96 (d, J = 5.5 Hz, 1H), 8.72 (d, J = 8.6 Hz, 1H), 8.48 (d, J = 1.3 Hz, 1H), 8.41-8.39 (d, 1H), 7.99 (dd, J = 1.6, 8.4 Hz, 1H), 7.78 (d, J = 2.1 Hz, 1H), 7.60-7.54 (m, 1H), 7.53-7.47 (m, 1H), 4.31 (s, 2H), 3.87 (t, J = 6.6 Hz, |
| | carboxamide | 3-Chloro-4- | 2H), 3.42-3.36 (m, 4H), 2.78 (t, J = |
| | | (trifluoromethoxy) | 6.7 Hz, 2H), 2.11-2.03 (quintet, 2H) ppm. |
| | | benzaldehyde | LCMS (AM3): rt = 0.731 min, (575.0 |
| | | CAS 83279-39-4 | [M + H]⁺), 96.67% purity. |
| | | | Purification Method 126 |
| EXAMPLE 101 | 5-((3-(3-((3- Chloro-4- cyclopropoxybenzyl) amino)propanamido) propyl)amino) benzo[c][2,6]naph- thyridine-8- | 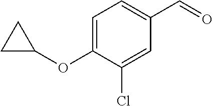 [SMILES: O=Cc1ccc(OC2CC2)c(Cl)c1] | ¹H NMR (400 MHz, MeOH-d₄) δ: 10.04 (s, 1H), 8.96 (d, J = 5.6 Hz, 1H), 8.74 (d, J = 8.6 Hz, 1H), 8.46 (d, J = 1.5 Hz, 1H), 8.38 (d, J = 5.6 Hz, 1H), 7.99 (dd, J = 1.7, 8.4 Hz, 1H), 7.52 (d, J = 1.8 Hz, 1H), 7.46-7.37 (m, 2H), 4.19 (s, 2H), 3.89-3.85 (m, 3H), 3.40 (t, J = 6.5 Hz, |
| | carboxamide | 3-Chloro-4- | 2H), 3.34-3.32 (m, 2H), 2.75 (t, J = |
| | | cyclopropoxybenz- | 6.7 Hz, 2H), 2.09-2.03 (m, 2H), 0.85-0.82 |
| | | aldehyde | (m, 2H), 0.75-0.72 (m, 2H) ppm. |
| | | (WO2022185041) | LCMS (AM3): rt = 0.739 min, (547.2 |
| | | | [M + H]⁺), 100% purity. |
| | | | Purification Method 116 |

### Example 102

### 5-((3-(3-((3-Chloro-4-cyclopropylbenzyl)amino)propanamido)propyl)amino)benzo[c][2,6]naphthyridine-8-carboxamide

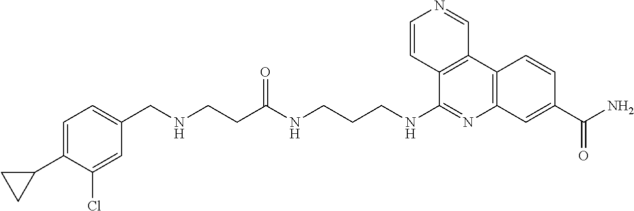 [SMILES: NC(=O)c1ccc2c(c1)nc(NCCCNC(=O)CCNCc1ccc(C3CC3)c(Cl)c1)c1ccncc12]

A mixture of Intermediate C (130 mg, 0.277 mmol) and 3-Chloro-4-cyclopropylbenzaldehyde (WO2022185041, 65 mg, 0.36 mmol) in MeOH (10 mL) was stirred at 35° C. for 15 h, then NaBH(OAc)₃ (234 mg, 1.10 mmol) was added. The reaction mixture was stirred at 35° C. for 2 h. The reaction mixture was concentrated in vacuo and the crude product was purified (PM116) to afford EXAMPLE 102 (48.22 mg, 27% yield, TFA salt) as a yellow solid.

LCMS (AM1): rt=0.742 min, (531.1 [M+H]⁺), 100% purity.

¹H NMR (400 MHz, MeOH-d₄) δ: 10.06 (s, 1H), 8.96 (d, J=5.6 Hz, 1H), 8.75 (d, J=8.4 Hz, 1H), 8.46 (d, J=1.6 Hz, 1H), 8.38 (d, J=5.6 Hz, 1H), 8.00 (dd, J=8.4 Hz, 1.6 Hz, 1H), 7.52 (d, J=1.6 Hz, 1H), 7.31 (dd, J=8.0 Hz, 1.6 Hz, 1H), 7.04 (d, J=8.0 Hz, 1H), 4.20 (s, 2H), 3.87 (t, J=6.8 Hz, 2H), 3.40 (t, J=6.4 Hz, 2H), 3.34-3.32 (m, 2H), 2.75 (t, J=6.8 Hz, 2H), 2.24-2.16 (m, 1H), 2.13-2.03 (quintet, 2H), 1.07-1.02 (m, 2H), 0.71-0.66 (m, 2H) ppm.

The following examples in Table 5 were made with non-critical changes or substitutions to the exemplified procedure in EXAMPLE 102, that would be understood by one skilled in the art using Intermediate C and compounds of formula (Ic).

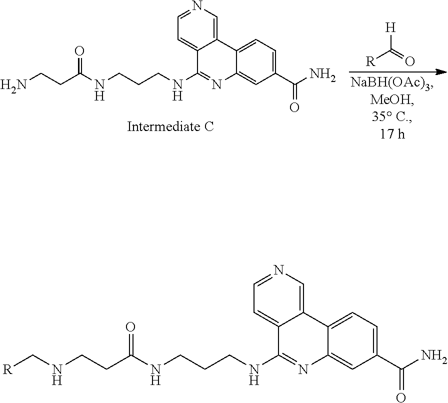

TABLE 5

| Example | Chemical | | |
|---|---|---|---|
| No. | IUPAC name | Compound (Ic) | Analytical |
| EXAMPLE 103 | 5-((3-(3-((3- chloro-4- (cyclopentyl- oxy)benzyl) amino)propan- amido)propyl) amino)benzo [c][2,6]naph- thyridine-8- carboxamide | 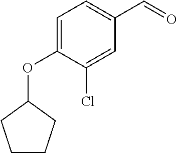 [SMILES: O=Cc1ccc(OC2CCCC2)c(Cl)c1] | ¹H NMR (400 MHz, MeOH-d₄) δ: 10.06 (s, 1H), 8.95 (d, J = 5.6 Hz, 1H), 8.75 (d, J = 8.4 Hz, 1H), 8.44 (d, J = 1.6 Hz, 1H), 8.35 (d, J = 5.6 Hz, 1H), 7.99 (dd, J = 8.4 Hz, 1.6 Hz, 1H), 7.52 (d, J = 2.0 Hz, 1H), 7.35 (dd, J = 8.4 Hz, 2.4 Hz, 1H), 7.08 (d, J = 8.4 Hz, 1H), 4.90-4.84 (m, 1H), 4.17 (s, 2H), 3.86 (t, J = 6.8 Hz, 2H), 3.40 (t, J = 6.4 Hz, 2H), 3.33-3.31 (m, 2 H), 2.74 (t, J = 6.8 Hz, 2H), 2.11-2.10 (m, |
| | | 3-chloro-4- | 2H), 1.97-1.87 (m, 2H), 1.87-1.75 (m, |
| | | (cyclopentyloxy) | 4H), 1.73-1.61 (m, 2H) ppm. |
| | | benzaldehyde | LCMS (AM3): rt = 0.784 min, (575.2 |
| | | (WO2022185041) | [M + H]⁺), 100% purity. |
| | | | Purification Method 156 |
| EXAMPLE 104 | 5-((3-(3-((3- chloro-4- cyclobutoxy- benzyl)amino) propanamido) propyl)amino) benzo[c][2,6] naphthyridine- 8- | 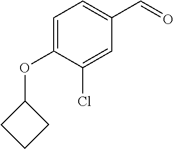 [SMILES: O=Cc1ccc(OC2CCC2)c(Cl)c1] | ¹H NMR (400 MHz, MeOH-d₄) δ: 10.04 (s, 1H), 8.94 (d, J = 5.6 Hz, 1H), 8.73 (d, J = 8.4 Hz, 1H), 8.44 (s, 1H), 8.36 (d, J = 5.6 Hz, 1H), 7.98 (dd, J = 8.4 Hz, 1.6 Hz, 1H), 7.52 (d, J = 2.0 Hz, 1H), 7.34 (dd, J = 8.4 Hz, 2.4 Hz, 1H), 6.93 (d, J = 8.8 Hz, 1H), 4.77-4.84 (m, 1H), 4.17 (s, 2H), 3.86 (t, J = 6.8 Hz, 2H), 3.40 (t, J = 6.8 Hz, 2H), 3.35-3.30 (m, 2 H), 2.74 (t, |
| | carboxamide | 3-chloro-4- | J = 6.8 Hz, 2H), 2.50-2.45 (m, 2H), |
| | | cyclobutoxybenz- | 2.20-2.11 (m, 2H), 2.11-2.03 (m, 2H), |
| | | aldehyde | 1.91-1.83 (m, 1H), 1.80-1.68 (m, 1H) |
| | | (WO2022185041) | ppm. |
| | | | LCMS (AM1): rt = 0.762 min, (561.2 |
| | | | [M + H]⁺), 100% purity. |
| | | | Purification Method 147 |
| EXAMPLE 105 | 5-((3-(3-(((6- Chloro-1H- indol-2- yl)methyl) amino)propan- amido)propyl) | 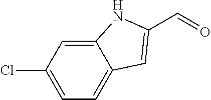 [SMILES: O=Cc1cc2ccc(Cl)cc2[nH]1] | ¹H NMR (400 MHz, MeOH-d₄) δ 10.05 (s, 1H), 8.94 (d, J = 5.6 Hz, 1H), 8.73 (d, J = 8.6 Hz, 1H), 8.43 (d, J = 1.5 Hz, 1H), 8.33 (d, J = 5.6 Hz, 1H), 7.99 (dd, J = 1.7, 8.4 Hz, 1H), 7.45 (d, J = 8.4 Hz, 1H), 7.34 (s, 1H), 6.96 (dd, J = 1.9, 8.5 Hz, |
| | amino)benzo | 6-chloro-1H- | 1H), 6.65 (s, 1H), 4.43 (s, 2H), 3.83 (t, J = |
| | [c][2,6]naph- | indole-2- | 6.5 Hz, 2H), 3.41-3.36 (m, 4H), 2.76 |
| | thyridine-8- | carbaldehyde | (t, J = 6.5 Hz, 2H), 2.04 (quintet, J = 6.5 Hz, |
| | carboxamide | CAS 53590-59-3 | 2H) ppm. |
| | | | LCMS (AM3): rt = 0.745 min, (530.3 |
| | | | [M + H]⁺), 100% purity. |
| | | | Purification Method 157 |
| EXAMPLE 106 | 5-((3-(3-((3- Fluoro-4- (trifluorometh- oxy)benzyl) amino)propan- amido)propyl) amino)benzo | 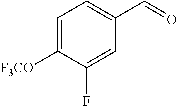 [SMILES: O=Cc1ccc(OC(F)(F)F)c(F)c1] | ¹H NMR (400 MHz, MeOH-d₄) δ: 10.07 (s, 1H), 8.97 (d, J = 5.7 Hz, 1H), 8.77 (d, J = 8.6 Hz, 1H), 8.48 (d, J = 1.5 Hz, 1H), 8.38 (d, J = 5.6 Hz, 1H), 8.01 (dd, J = 1.7, 8.5 Hz, 1H), 7.57-7.47 (m, 2H), 7.43-7.41 (d, 1H), 4.32 (s, 2H), 3.87 (t, J = 6.6 Hz, 2H), 3.43-3.35 (m, 4H), 2.77 |
| | [c][2,6]naph- | 3-fluoro-4- | (t, J = 6.7 Hz, 2H), 2.10-2.02 (quintet, |
| | thyridine-8- | (trifluoromethoxy) | 2H) ppm. |
| | carboxamide | benzaldehyde | LCMS (AM3): rt = 0.737 min, (559.3 |
| | | CAS 473917-15-6 | [M + H]⁺), 100% purity. |
| | | | Purification Method 127 |
| EXAMPLE 107 | 5-((3-(3-((3,4- Dichlorobenzyl) amino)pro- panamido) propyl)amino) benzo[c][2,6] naphthyridine- | 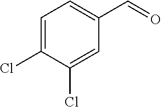 | ¹H NMR (400 MHz, MeOH-d₄) δ: 10.06 (s, 1H), 8.96 (d, J = 5.7 Hz, 1H), 8.75 (d, J = 8.4 Hz, 1H), 8.47 (d, J = 1.3 Hz, 1H), 8.38 (d, J = 5.6 Hz, 1H), 8.00 (dd, J = 1.7, 8.4 Hz, 1H), 7.70 (d, J = 2.1 Hz, 1H), 7.60 (d, J = 8.3 Hz, 1H), 7.45-7.43 (dd, 1H), 4.26 (s, 2H), 3.87 (t, J = 6.6 Hz, 2H), |
| | 8- | 3,4- | 3.40 (t, J = 6.5 Hz, 2H), 3.36 (t, J = 6.7 Hz, |
| | carboxamide | dichlorobenz- | 2H), 2.77 (t, J = 6.6 Hz, 2H), 2.07 |
| | | aldehyde | (quintet, J = 6.6 Hz, 2H) ppm. |
| | | CAS 6287-38-3 | LCMS (AM3): rt = 0.736 min, (525.2 |
| | | | [M + H]⁺), 100% purity. |
| | | | Purification Method 158 |
| EXAMPLE 108 | 5-((3-(3-((3,5- Dichlorobenzyl) amino)pro- panamido) propyl)amino) benzo[c][2,6] naphthyridine- | 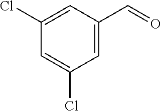 | ¹H NMR (400 MHz, MeOH-d₄) δ: 10.06 (s, 1H), 8.96 (d, J = 5.6 Hz, 1H), 8.75 (d, J = 8.8 Hz, 1H), 8.45 (d, J = 1.6 Hz, 1H), 8.37 (d, J = 5.6 Hz, 1H), 7.99 (dd, J = 1.6, 8.4 Hz, 1H), 7.55 (m, 1H), 7.50 (d, J = 1.6 Hz, 2H), 4.26 (s, 2H), 3.87 (t, J = 6.4 Hz, 2H), 3.41 (t, J = 6.4 Hz, 2H), 3.36 |
| | 8- | 3,5- | (t, J = 6.8 Hz, 2H), 2.77 (t, J = 6.8 Hz, |
| | carboxamide | dichlorobenz- | 2H), 2.10-2.03 (quintet, 2H) ppm. |
| | | aldehyde CAS | LCMS (AM3): rt = 0.734 min, (525.2 |
| | | 10203-08-4 | [M + H]⁺), 100% purity. |
| | | | Purification Method 158 |
| EXAMPLE 109 | 5-((3-(3-((3- Chloro-4- (trifluoromethyl) benzyl)amino) propanamido) propyl)amino) benzo[c] | 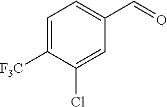 [SMILES: O=Cc1ccc(C(F)(F)F)c(Cl)c1] | ¹H NMR (400 MHz, MeOH-d₄) δ: 10.09 (s, 1H), 8.98 (d, J = 5.6 Hz, 1H), 8.78 (d, J = 8.4 Hz, 1H), 8.50 (d, J = 1.6 Hz, 1H), 8.39 (d, J = 5.6 Hz, 1H), 8.03 (dd, J = 1.6, 8.4 Hz, 1H), 7.86 (d, J = 8 Hz, 1H), 7.80 (s, 1H), 7.62 (d, J = 8.8 Hz, 1H), 4.36 (s, 2H), 3.88 (t, J = 6.8 Hz, 2H), |
| | [2,6]naph- | 3-chloro-4- | 3.43-3.40 (m, 4H), 2.78 (t, J = 6.8 Hz, |
| | thyridine-8- | (trifluoromethyl) | 2H), 2.11-2.05 (quintet, 2H) ppm. |
| | carboxamide | benzaldehyde | LCMS (AM3): rt = 0.748 min, (559.3 |
| | | CAS 83279-38-3 | [M + H]⁺), 100% purity. |
| | | | Purification Method 159 |
| EXAMPLE 110 | 5-((3-(3-((3- Chloro-5- fluorobenzyl) amino)prop- anamido)propyl) amino)benzo [c][2,6]naph- | 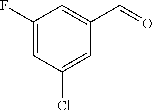 | ¹H NMR (400 MHz, MeOH-d₄) δ: 10.07 (s, 1H), 8.97 (d, J = 5.6 Hz, 1H), 8.77 (d, J = 8.5 Hz, 1H), 8.47 (s, 1H), 8.38 (d, J = 5.6 Hz, 1H), 8.01 (dd, J = 1.6, 8.4 Hz, 1H), 7.41 (s, 1H), 7.32 (td, J = 2.0, 8.6 Hz, 1H), 7.26 (d, J = 8.9 Hz, 1H), 4.28 (s, 2H), 3.87 (t, J = 6.6 Hz, 2H), 3.41 (t, |
| | thyridine-8- | 3-chloro-5-fluoro- | J = 6.5 Hz, 2H), 3.38-3.34 (t, 2H), 2.77 |
| | carboxamide | benzaldehyde | (t, J = 6.7 Hz, 2H), 2.07 (quintet, J = |
| | | CAS 90390- | 6.9 Hz, 2H) ppm. |
| | | 49-1 | LCMS (AM3): rt = 0.699 min, (509.2 |
| | | | [M + H]⁺), 99.7% purity. |
| | | | Purification Method 160 |
| EXAMPLE 111 | 5-((3-(3-(((6- Chloro-1- methyl-1H- indol-2- yl)methyl) amino)propan- amido)propyl) | 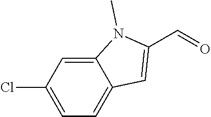 | ¹H NMR (400 MHz, MeOH-d₄) δ: 10.02 (s, 1H), 8.90 (d, J = 5.8 Hz, 1H), 8.69 (d, J = 8.3 Hz, 1H), 8.36 (s, 1H), 8.27 (d, J = 5.3 Hz, 1H), 7.92 (d, J = 7.2 Hz, 1H), 7.47 (d, J = 8.5 Hz, 1H), 7.43 (s, 1H), 7.02 (dd, J = 1.8, 8.5 Hz, 1H), 6.70 (s, 1H), 4.53 (s, 2H), 3.82 (t, J = 6.5 Hz, 2H), |
| | amino)benzo | 6-chloro-1- | 3.77 (s, 3H), 3.45-3.37 (m, 4H), 2.78 (t, |
| | [c][2,6]naph- | methyl-1H- | J = 6.5 Hz, 2H), 2.07-2.00 (m, 2H) ppm. |
| | thyridine-8- | indole-2- | LCMS (AM3): rt = 0.741 min, (544.2 |
| | carboxamide | carbaldehyde | [M + H]⁺), 100% purity. |
| | | CAS 1781757- | Purification Method 161 |
| | | 70-7 | |
| EXAMPLE 112 | 5-((3-(3-(((4- Chloro-3- phenyl-1H- pyrazol-5- yl)methyl)amino) propanamido) propyl) amino)benzo[c] | 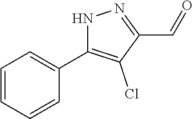 [SMILES: O=Cc1n[nH]c(-c2ccccc2)c1Cl] | ¹H NMR (400 MHz, MeOH-d₄-d4) δ: 10.05 (s, 1H), 8.96 (d, J = 5.6 Hz, 1H), 8.74 (d, J = 8.8 Hz, 1H), 8.47 (d, J = 1.6 Hz, 1H), 8.39 (d, J = 5.6 Hz, 1H), 8.00 (dd, J = 1.6, 8.8 Hz, 1H), 7.71-7.69 (m, 2H), 7.49-7.45 (m, 3H), 4.36 (s, 2H), 3.87 (t, J = 6.8 Hz, 2H), 3.50-3.47 (t, 2H), 3.44-3.41 (t, 2H), 2.81 (t, J = 6.8 Hz, |
| | [2,6]naph- | 4-chloro-5- | 2H), 2.08 (quintet, J = 6.8 Hz, 2H) |
| | thyridine-8- | phenyl-1H- | ppm. |
| | carboxamide | pyrazole-3- | LCMS (AM3): rt = 0.724 min, (557.2 |
| | | carbaldehyde | [M + H]⁺), 98.03% purity. |
| | | 1.67 | Purification Method 162 |
| EXAMPLE 113 | 5-((3-(3-(((4- Chloro-1- methyl-3- phenyl-1H- pyrazol-5- yl)methyl) amino)propan- amido)propyl) amino)benzo | 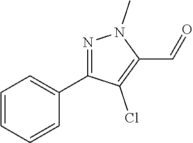 | LCMS (AM3): rt = 0.624 min, (571.3 [M + H]⁺), 100% purity. ¹H NMR (400 MHz, MeOH-d₄) δ: 10.00 (s, 1H), 8.94 (d, J = 5.6 Hz, 1H), 8.69 (d, J = 8.8 Hz, 1H), 8.44 (d, J = 1.6 Hz, 1H), 8.36 (d, J = 5.6 Hz, 1H), 7.97 (dd, J = 1.6, 8.4 Hz, 1H), 7.78-7.75 (m, 2H), 7.42-7.39 (m, 3H), 4.51 (s, 2H), 4.01 (s, 3H), 3.85 (t, J = 6.8 Hz, 2H), 3.49-3.46 |
| | [c][2,6]naph- | 4-chloro-1- | (t, 2H), 3.42-3.39 (t, 2H), 2.81 (t, J = |
| | thyridine-8- | methyl-3- | 6.4 Hz, 2H), 2.07 (quintet, J = 6.4 Hz, 2H) |
| | carboxamide | phenyl-1H- | ppm. |
| | | pyrazole-5- | Purification Method 163 |
| | | carbaldehyde | |
| | | CAS 1780286- | |
| | | 92-1 | |
| EXAMPLE 114 | 5-((3-(3-(((6- (Trifluoromethyl) pyridin-3- yl)methyl)amino) propanamido) | 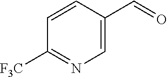 [SMILES: O=Cc1ccc(C(F)(F)F)nc1] | ¹H NMR (400 MHz, MeOH-d₄) δ: 10.06 (s, 1H), 8.95 (d, J = 5.6 Hz, 1H), 8.86 (d, J = 1.6 Hz, 1H), 8.75 (d, J = 8.5 Hz, 1H), 8.44 (d, J = 1.4 Hz, 1H), 8.34 (d, J = 5.6 Hz, 1H), 8.20 (dd, J = 1.7, 8.1 Hz, 1H), |
| | propyl) | 6- | 7.98 (dd, J = 1.6, 8.4 Hz, 1H), 7.91 (d, J = |
| | amino)benzo | (trifluoromethyl) | 8.0 Hz, 1H), 4.45 (s, 2H), 3.87 (t, J = |
| | [c][2,6]naph- | nicotinaldehyde | 6.7 Hz, 2H), 3.41 (q, J = 6.9 Hz, 4H), |
| | thyridine-8- | CAS 386704-12- | 2.80 (t, J = 6.6 Hz, 2H), 2.09-2.03 |
| | carboxamide | 7 | (quintet, 2H) ppm. |
| | | | LCMS (AM3): rt = 0.672 min, (526.2 |
| | | | [M + H]⁺), 100% purity. |
| | | | Purification Method 164 |
| EXAMPLE 115 | 5-((3-(3-(((5- Chloropyridin- 3- yl)methyl) amino)propan- amido)propyl) amino)benzo | 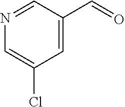 | ¹H NMR (400 MHz, MeOH-d₄) δ 10.07 (br s, 1H), 8.98 (d, J = 5.3 Hz, 1H), 8.76 (d, J = 8.5 Hz, 1H), 8.66 - 8.63 (m, 2H), 8.49 (d, J = 1.5 Hz, 1H), 8.40 (d, J = 5.8 Hz, 1H), 8.06 (t, J = 2.0 Hz, 1H), 8.01 (dd, J = 1.6, 8.4 Hz, 1H), 4.36 (s, 2H), 3.88 (t, J = 6.7 Hz, 2H), 3.41 (t, J = 6.7 Hz, |
| | [c][2,6]naph- | 5- | 4H), 2.79 (t, J = 6.7 Hz, 2H), 2.08 |
| | thyridine-8- | chloronicotinalde- | (quintet, J = 6.5 Hz, 2H) ppm. |
| | carboxamide | hyde | LCMS (AM3): rt = 0.627 min, (492.2 |
| | | CAS 113118-82- | [M + H]⁺), 96.4% purity. |
| | | 4 | Purification Method 165 |
| EXAMPLE 116 | 5-((3-(3-(((5- (Trifluoromethyl) pyridin-2- yl)methyl)amino) propan- | 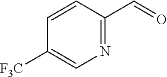 [SMILES: O=Cc1ccc(C(F)(F)F)cn1] | ¹H NMR (400 MHz, MeOH-d₄) δ: 10.06 (s, 1H), 8.94 (d, J = 5.7 Hz, 1H), 8.90 (s, 1H), 8.74 (d, J = 8.4 Hz, 1H), 8.44 (d, J = 1.5 Hz, 1H), 8.35 (d, J = 5.6 Hz, 1H), 8.19 (dd, J = 2.0, 8.2 Hz, 1H), 7.97 (dd, |
| | amido)propyl) | 5- | J = 1.8, 8.5 Hz, 1H), 7.66 (d, J = 8.2 Hz, |
| | amino)benzo[c] | (trifluoromethyl) | 1H), 4.56 (s, 2H), 3.88 (t, J = 6.7 Hz, 2H), |
| | [2,6]naph- | picolinaldehyde | 3.49-3.46 (t, 2H), 3.42 (t, J = 6.6 Hz, |
| | thyridine-8- | CAS 31224-82-5 | 2H), 2.85-2.81 (t, 2H), 2.11-2.04 |
| | carboxamide | | (quintet, 2H) ppm. |
| | | | LCMS (AM3): rt = 0.688 min, (526.2 |
| | | | [M + H]⁺), 100% purity. |
| | | | Purification Method 166 |
| EXAMPLE 117 | 5-((3-(3-((3- Chloro-4- cyclopentyl- benzyl)amino) propanamido) propyl)amino) benzo[c][2,6] | 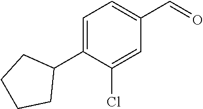 [SMILES: O=Cc1ccc(C2CCCC2)c(Cl)c1] | ¹H NMR (400 MHz, MeOH-d₄) δ: 10.06 (s, 1H), 8.96-8.93 (m, 1H), 8.75 (d, J = 8.0 Hz, 1H), 8.47-8.29 (m, 2H), 7.98-7.96 (m, 1H), 7.51 (d, J = 1.8 Hz, 1H), 7.44-7.41 (m, 1H), 7.37-7.34 (m, 1H), 4.21 (s, 2H), 3.90-3.81 (m, 2H), 3.49-3.33 (m, 5H), 2.75 (t, J = 6.7 Hz, |
| | naphthyridine- | 3-chloro-4- | 2H), 2.09-2.01 (m, 4H), 1.88-1.78 (m, |
| | 8- | cyclopentylbenz- | 2H), 1.77-1.68 (m, 2H), 1.62-1.50 (m, |
| | carboxamide | aldehyde 1.100 | 2H) ppm. |
| | | | LCMS (AM3): rt = 0.751 min, (559.0 |
| | | | [M + H]⁺), 99.85% purity |
| | | | Purification Method 45 |
| EXAMPLE 118 | 5-((3-(3-((3- (2- Hydroxyethyl) benzyl)amino) propanamido) | 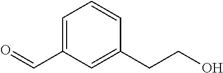 [SMILES: O=Cc1cccc(CCO)c1] | ¹H NMR (400 MHz, MeOH-d₄) δ: 10.07 (s, 1H), 8.96 (d, J = 5.8 Hz, 1H), 8.76 (d, J = 8.5 Hz, 1H), 8.47 (s, 1H), 8.37 (d, J = 5.8 Hz, 1H), 8.01 (d, J = 8.5 Hz, 1H), 7.41-7.30 (m, 4H), 4.23 (s, 2H), 3.87 (t, |
| | propyl)amino) | 3-(2- | J = 6.7 Hz, 2H), 3.77 (t, J = 6.8 Hz, 2H), |
| | benzo[c] | hydroxyethyl) | 3.40 (t, J = 6.5 Hz, 2H), 3.35-3.32 (m, |
| | [2,6]naph- | benzaldehyde CAS | 2H), 2.84 (t, J = 6.8 Hz, 2H), 2.74 (t, J = |
| | thyridine-8- | 212914-87-9 | 6.8 Hz, 2H), 2.07 (quintet, J = 6.6 Hz, |
| | carboxamide | | 2H) ppm. |
| | | | LCMS (AM3): rt = 0.602 min, (501.0 |
| | | | [M + H]⁺), 100% purity. |
| | | | Purification Method 167 |
| EXAMPLE 119 | 5-((3-(3-((3- (Hydroxymethyl) benzyl) amino)propan- amido)propyl) amino)benzo [c][2,6]naph- thyridine-8- | 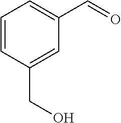 | ¹H NMR (400 MHz, MeOH-d₄) δ: 10.07 (s, 1H), 8.97 (d, J = 5.8 Hz, 1H), 8.76 (d, J = 8.5 Hz, 1H), 8.48 (d, J = 1.6 Hz, 1H), 8.39 (d, J = 5.8 Hz, 1H), 8.01 (dd, J = 1.8, 8.4 Hz, 1H), 7.48 (s, 1H), 7.45-7.36 (m, 3H), 4.63 (s, 2H), 4.26 (s, 2H), 3.87 (t, J = 6.7 Hz, 2H), 3.40 (t, J = 6.6 Hz, 2H), 3.36-3.32 (t, 2H), 2.75 (t, J = 6.8 Hz, |
| | carboxamide | 3- | 2H), 2.07 (quintet, J = 6.6 Hz, 2H) |
| | | (hydroxymethyl) | ppm. |
| | | benzaldehyde | LCMS (AM3): rt = 0.577 min, (487.0 |
| | | CAS 52010-98-7 | [M + H]⁺), 98.4% purity. |
| | | | Purification Method 168 |
| EXAMPLE 120 | 5-((3-(3-((3- (2- Hydroxyethoxy) benzyl)amino) propanamido) propyl) amino)benzo [c][2,6]naph- thyridine-8- carboxamide | 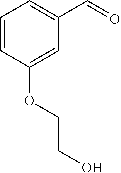 [SMILES: O=Cc1cccc(OCCO)c1] | ¹H NMR (400 MHz, MeOH-d₄) δ: 10.06 (s, 1H), 8.96 (d, J = 5.6 Hz, 1H), 8.76 (d, J = 8.5 Hz, 1H), 8.47 (d, J = 1.5 Hz, 1H), 8.38 (d, J = 5.8 Hz, 1H), 8.00 (dd, J = 1.7, 8.4 Hz, 1H), 7.35 (t, J = 7.9 Hz, 1H), 7.08-7.03 (m, 3H), 4.22 (s, 2H), 4.07-4.04 (t, 2H), 3.88-3.85 (t, 4H), 3.40 (t, J = 6.5 Hz, 2H), 3.35-3.32 (m, 2H), 2.75 (t, J = 6.7 Hz, 2H), 2.07 (quintet, J = 6.4 Hz, 2H) ppm. LCMS (AM3): rt = 0.597 min, (517.0 |
| | | 3-(2- | [M + H]⁺), 99.1% purity. |
| | | hydroxyethoxy) | Purification Method 168 |
| | | benzaldehyde | |
| | | CAS 60345-97-3 | |
| EXAMPLE 121 | 5-((3-(3-((3- Cyanobenzyl) amino)prop- anamido)propyl) amino)benzo [c][2,6]naph- thyridine-8- | 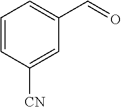 | ¹H NMR (400 MHz, MeOH-d₄-d4) δ: 10.07 (s, 1H), 8.98 (d, J = 5.6 Hz, 1H), 8.76 (d, J = 8.8 Hz, 1H), 8.49 (d, J = 1.2 Hz, 1H), 8.41 (d, J = 5.6 Hz, 1H), 8.01 (dd, J = 1.6, 8.8 Hz, 1H), 7.89 (s, 1H), 7.82-7.80 (m, 2H), 7.66-7.62 (m, 1H), 4.35 (s, 2H), 3.88 (t, J = 6.8 Hz, 2H), |
| | carboxamide | 3- | 3.43-3.36 (m, 4H), 2.78 (t, J = 6.8 Hz, |
| | | formylbenzonitrile | 2H), 2.10-2.07 (quintet, 2H) ppm. |
| | | CAS 24964- | LCMS (AM3): rt = 0.602 min, (481.9 |
| | | 64-5 | [M + H]⁺), 100.00% purity. |
| | | | Purification Method 169 |
| EXAMPLE 122 | 5-((3-(3-((3- Chloro-4- cyclopentyl- benzyl)amino) propanamido) propyl)amino) benzo[c][2,6] | 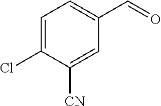 [SMILES: N#Cc1cc(C=O)ccc1Cl] | ¹H NMR (400 MHz, MeOH-d₄) δ: 10.05 (s, 1H), 8.97 (d, J = 5.6 Hz, 1H), 8.74 (d, J = 8.8 Hz, 1H), 8.48 (d, J = 1.6 Hz, 1H), 8.41 (d, J = 5.6 Hz, 1H), 8.01 (dd, J = 1.6, 8.8 Hz, 1H), 7.95 (d, J = 2.0 Hz, 1H), 7.82-7.76 (m, 1H), 7.71-7.69 (m, 1H), 4.33 (s, 2H), 3.88 (t, J = 6.8 Hz, 2H), 3.40 |
| | naphthyridine- | 2-chloro-5- | (td, J = 6.4, 10.4 Hz, 4H), 2.78 (t, J = |
| | 8- | formylbenzonitrile | 6.8 Hz, 2H), 2.12-2.05 (quintet, 2H) ppm. |
| | carboxamide | CAS 1261759- | LCMS (AM3): rt = 0.662 min, (515.9 |
| | | 41-4 | [M + H]⁺), 100% purity Purification |
| | | | Method 170 |
| EXAMPLE 123 | 5-((3-(3-(((6- Methylpyridin- 2- yl)methyl)amino) propanamido) propyl) amino)benzo | 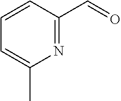 | ¹H NMR (400 MHz, MeOH-d₄) δ: 9.91 (s, 1H), 8.77 (d, J = 5.6 Hz, 1H), 8.59 (d, J = 8.4 Hz, 1H), 8.19 (d, J = 1.8 Hz, 1H), 8.13 (d, J = 5.6 Hz, 1H), 7.81 (dd, J = 1.9, 8.4 Hz, 1H), 7.58 (t, J = 7.7 Hz, 1H), 7.18 (d, J = 7.8 Hz, 1H), 7.07 (d, J = 7.8 Hz, 1H), 3.83 (s, 2H), 3.75 (t, J = 6.7 Hz, |
| | [c][2,6]naph- | 6- | 2H), 3.39-3.35 (m, 2H), 2.91 (t, J = 6.7 Hz, |
| | thyridine-8- | methylpicolinalde- | 2H), 2.48 (t, J = 6.8 Hz, 2H), 2.45 (s, |
| | carboxamide | hyde CAS | 3H), 1.97 (quintet, J = 6.7 Hz, 2H) ppm. |
| | | 1122-72-1 | LCMS (AM5): rt = 0.765 min, (472.4 |
| | | | [M + H]⁺), 95.8% purity |
| | | | Purification Method 171 |
| EXAMPLE 124 | 5-((3-(3-((3- Cyano-4- (trifluorometh- oxy)benzyl) amino)propan- amido)propyl) amino)benzo [c][2,6]naph- thyridine-8- | 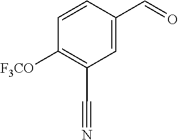 [SMILES: N#Cc1cc(C=O)ccc1OC(F)(F)F] | ¹H NMR (400 MHz, MeOH-d₄) δ: 10.06 (s, 1H), 8.95 (d, J = 5.8 Hz, 1H), 8.75 (d, J = 8.7 Hz, 1H), 8.44 (d, J = 1.5 Hz, 1H), 8.35 (d, J = 5.8 Hz, 1H), 8.03 (d, J = 2.3 Hz, 1H), 7.98 (dd, J = 1.7, 8.5 Hz, 1H), 7.93 (dd, J = 2.3, 8.7 Hz, 1H), 7.65 (dd, J = 1.4, 8.7 Hz, 1H), 4.37 (s, 2H), 3.87 (t, J = 6.7 Hz, 2H), 3.42-3.37 (q, 4H), 2.79 (t, J = 6.6 Hz, 2H), 2.11-2.02 (quin, 2H) |
| | carboxamide | 5-Formyl-2- | ppm. |
| | | (trifluoromethoxy) | LCMS (AM3): rt = 0.729 min, (566.3 |
| | | benzonitrile, | [M + H]⁺), 100% purity. |
| | | (WO2022185041) | Purification Method 116 |
| EXAMPLE 125 | 5-((3-(3-((3- Chloro-5- (hydroxymethyl) benzyl)amino) propanamido) propyl) amino)benzo [c][2,6]naph- | 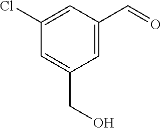 [SMILES: O=Cc1cc(Cl)cc(CO)c1] | ¹H NMR (400 MHz, MeOH-d₄) δ: 10.08 (s, 1H), 8.98 (d, J = 5.8 Hz, 1H), 8.78 (d, J = 8.6 Hz, 1H), 8.49 (d, J = 1.3 Hz, 1H), 8.40 (d, J = 5.6 Hz, 1H), 8.02 (dd, J = 1.6, 8.4 Hz, 1H), 7.44 (d, J = 6.6 Hz, 2H), 7.40 (s, 1H), 4.62 (s, 2H), 4.26 (s, 2H), 3.88 (t, J = 6.6 Hz, 2H), 3.41 (t, J = 6.5 Hz, 2H), 3.35 (t, J = 6.7 Hz, 2H), 2.76 (t, |
| | thyridine-8- | 3-chloro-5- | J = 6.7 Hz, 2H), 2.08 (quintet, J = 6.6 Hz, |
| | carboxamide | (hydroxymethyl) | 2H) ppm. |
| | | benzaldehyde | LCMS (AM3): rt = 0.604 min, (521.1 |
| | | (WO2022185041) | [M + H]⁺), 95.91% purity. |
| | | | Purification Method 117 |
| EXAMPLE 126 | 5-((3-(3-((3- (Cyanomethyl) benzyl)amino) propanamido) propyl)amino) benzo[c] [2,6]naph- thyridine-8- carboxamide | 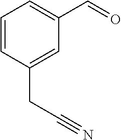 [SMILES: N#CCc1cccc(C=O)c1] | ¹H NMR (400 MHz, MeOH-d₄) δ: 10.09 (s, 1H), 9.00 (d, J = 5.6 Hz, 1H), 8.78 (d, J = 8.5 Hz, 1H), 8.55 (d, J = 1.6 Hz, 1H), 8.46 (d, J = 5.6 Hz, 1H), 8.05 (dd, J = 1.7, 8.4 Hz, 1H), 7.52 (s, 1H), 7.49-7.43 (m, 3H), 4.29 (s, 2H), 3.94 (s, 2H), 3.89 (t, J = 6.6 Hz, 2H), 3.41 (t, J = 6.4 Hz, 2H), 3.36 (t, J = 6.8 Hz, 2H), 2.77 (t, J = 6.8 Hz, 2H), 2.10 (quin, J = 6.6 Hz, 2H) |
| | | 2-(3- | ppm. |
| | | formylphenyl) | LCMS (AM3): rt = 0.542 min, (496.3 |
| | | acetonitrile | [M + H]⁺), 97.8% purity. |
| | | (WO2022185041) | Purification Method 170 |
| EXAMPLE 127 | 5-((3-(3-((3- cyano-4- cyclobutoxy- benzyl)amino) propanamido) propyl)amino) benzo[c][2,6] | 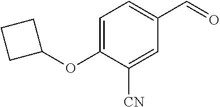 [SMILES: N#Cc1cc(C=O)ccc1OC1CCC1] | ¹H NMR (400 MHz, MeOH-d₄) δ: 10.05 (s, 1H), 8.96 (d, J = 5.6 Hz, 1H), 8.75 (d, J = 8.8 Hz, 1H), 8.46 (d, J = 1.6 Hz, 1H), 8.38 (d, J = 5.6 Hz, 1H), 7.99 (dd, J = 1.6, 8.4 Hz, 1H), 7.75 (d, J = 2.4 Hz, 1H), 7.69 (dd, J = 2.4, 8.8 Hz, 1H), 7.05 (d, J = 8.8 Hz, 1H), 4.84-4.79 (m, 1H), 4.23 |
| | naphthyridine- | 2-cyclobutoxy-5- | (s, 2H), 3.86 (t, J = 6.8 Hz, 2H), 3.40 (t, |
| | 8- | formylbenzonitrile | J = 6.8 Hz, 2H), 3.36-3.32 (m, 2H), 2.76 |
| | carboxamide | (WO2022185041) | (t, J = 6.8 Hz, 2H), 2.55-2.47 (m, 2H), |
| | | | 2.25-2.13 (m, 2H), 2.11-2.03 (m, 2H), |
| | | | 1.96-1.77 (m, 2H) ppm. |
| | | | LCMS (AM3): rt = 0.743 min, (552.4 |
| | | | [M + H]⁺), 100.00% purity. |
| | | | Purification Method 127 |
| EXAMPLE 128 | 5-((3-(3-(((2- cyano-[1,1′- biphenyl]-4- yl)methyl)amino) propanamido) propyl) amino)benzo | 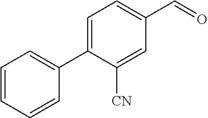 [SMILES: N#Cc1cc(C=O)ccc1-c1ccccc1] | ¹H NMR (400 MHz, MeOH-d₄) δ: 10.03 (s, 1H), 8.96 (d, J = 5.6 Hz, 1H), 8.72 (d, J = 8.8 Hz, 1H), 8.48 (d, J = 1.6 Hz, 1H), 8.40 (d, J = 5.6 Hz, 1H), 8.03-7.96 (m, 2H), 7.86 (dd, J = 2.0, 8.4 Hz, 1H), 7.65 (d, J = 8.4 Hz, 1H), 7.58-7.46 (m, 5H), 4.39 (s, 2H), 3.88 (t, J = 6.8 Hz, 2H), 3.42 |
| | [c][2,6]naph- | 4-formyl-[1,1′- | (t, J = 6.8 Hz, 4H), 2.81 (t, J = 6.8 Hz, |
| | thyridine-8- | biphenyl]-2- | 2H), 2.09 (t, J = 6.8 Hz, 2H) ppm. |
| | carboxamide | carbonitrile 1.49 | LCMS (AM3): rt = 0.749 min, (558.4 |
| | | | [M + H]⁺), 96.17% purity. |
| | | | Purification Method 127 |
| EXAMPLE 129 | 5-((3-(3-(((6- (trifluorometh- oxy)quinolin2- yl)methyl)amino) propanamido) | 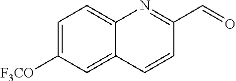 [SMILES: O=Cc1ccc2cc(OC(F)(F)F)ccc2n1] | ¹H NMR (400 MHz, MeOH-d₄) δ: 10.02 (s, 1H), 8.93 (d, J = 5.6 Hz, 1H), 8.68 (d, J = 8.4 Hz, 1H), 8.47-8.32 (m, 3H), 8.07 (d, J = 9.2 Hz, 1H), 7.98-7.81 (m, 2H), 7.69-7.55 (m, 2H), 4.67 (s, 2H), 3.90 (t, |
| | propyl) | 6- | J = 6.8 Hz, 2H), 3.57 (t, J = 6.5 Hz, 2H), |
| | amino)benzo | (trifluoromethoxy) | 3.45 (t, J = 6.8 Hz, 2H), 2.90 (t, J = 6.8 Hz, |
| | [c][2,6]naph- | quinoline-2- | 2H), 2.11 ( t, J = 6.8 Hz, 2H) ppm. |
| | thyridine-8- | carbaldehyde | LCMS (AM3): rt = 0.752 min, (592.3 |
| | carboxamide | 1.51 | [M + H]⁺), 99.75% purity. |
| | | | Purification Method 130 |
| EXAMPLE 130 | 5-((3-(3-((3- cyano-4- cyclopropyl- benzyl)amino) propanamido) propyl)amino) benzo[c][2,6] | 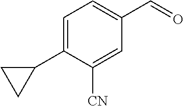 [SMILES: N#Cc1cc(C=O)ccc1C1CC1] | ¹H NMR: (400 MHz, MeOH-d₄) δ: 10.07 (s, 1H), 8.99 (d, J = 5.6 Hz, 1H), 8.77 (d, J = 8.4 Hz, 1H), 8.52 (d, J = 1.6 Hz, 1H), 8.45 (d, J = 5.6 Hz, 1H), 8.03 (dd, J = 1.6, 8.4 Hz, 1H), 7.78 (d, J = 2.0 Hz, 1H), 7.66 (dd, J = 2.0, 8.4 Hz, 1H), 7.11 (d, J = 8.4 Hz, 1H), 4.28 (s, 2H), 3.89 (t, J = |
| | naphthyridine- | 2-cyclopropyl-5- | 6.8 Hz, 2H), 3.46-3.33 (m, 4H), 2.77 (t, |
| | 8- | formylbenzonitrile | J = 6.8 Hz, 2H), 2.30-2.18 (m, 1H), 2.09 |
| | carboxamide | (WO2022185041) | (t, J = 6.4 Hz, 2H), 1.24-1.12 (m, 2H), |
| | | | 0.85-0.83 (m, 2H) ppm. |
| | | | LCMS (AM3): rt = 0.720 min, (522.2 |
| | | | [M + H]⁺), 98.25% purity. |
| | | | Purification Method 117 |
| EXAMPLE 131 | 5-((3-(3-(((6- Cyclopropyl- pyridin-3- yl)methyl)amino) propanamido) propyl) | 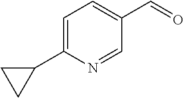 [SMILES: O=Cc1ccc(C2CC2)nc1] | ¹H NMR (400 MHz, MeOH-d₄) δ: 10.09 (s, 1H), 9.01 (d, J = 5.6 Hz, 1H), 8.78 (d, J = 8.6 Hz, 1H), 8.65 (s, 1H), 8.56 (d, J = 1.7 Hz, 1H), 8.47-8.46 (d, 1H), 8.14 (d, J = 8.3 Hz, 1H), 8.05 (dd, J = 1.6, 8.4 Hz, 1H), 7.49 (d, J = 8.3 Hz, 1H), 4.39 (s, |
| | amino)benzo | 6-cyclopropyl | 2H), 3.91 (t, J = 6.6 Hz, 2H), 3.43 (t, J = |
| | [c][2,6]naph- | nicotinaldehyde CAS | 6.5 Hz, 4H), 2.81 (t, J = 6.6 Hz, 2H), |
| | thyridine-8- | 75097-19-7 | 2.30-2.21 (m, 1H), 2.11 (quintet, J = |
| | carboxamide | | 6.4 Hz, 2H), 1.30-1.22 (m, 2H), 1.14-1.09 |
| | | | (m, 2H) ppm. |
| | | | LCMS (AM3): rt = 0.261 min, (498.1 |
| | | | [M + H]⁺), 100% purity. |
| | | | Purification Method 170 |
| EXAMPLE 132 | 5-((3-(3-(((5- Cyclopropyl- pyridin-2- yl)methyl) amino)propan- amido)propyl) | 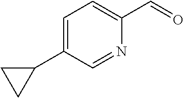 [SMILES: O=Cc1ccc(C2CC2)cn1] | ¹H NMR (400 MHz, MeOH-d₄) δ: 10.06 (s, 1H), 8.99 (d, J = 5.6 Hz, 1H), 8.75 (d, J = 8.6 Hz, 1H), 8.52 (d, J = 1.7 Hz, 1H), 8.47 (d, J = 5.6 Hz, 1H), 8.37 (d, J = 2.2 Hz, 1H), 8.02 (dd, J = 1.7, 8.6 Hz, 1H), 7.47 (dd, J = 2.2, 8.1 Hz, 1H), 7.33 (d, J = |
| | amino)benzo | 5- | 8.1 Hz, 1H), 4.35 (s, 2H), 3.90 (t, J = |
| | [c][2,6]naph- | cyclopropylpicolin- | 6.7 Hz, 2H), 3.45-3.40 (m, 4H), 2.81 (t, |
| | thyridine-8- | aldehyde CAS | J = 6.6 Hz, 2H), 2.11 (quintet, J = 6.5 Hz, |
| | carboxamide | 1256825-21-4 | 2H), 1.99-1.89 (m, 1H), 1.08-1.04 (m, |
| | | | 2H), 0.75-0.71 (m, 2H) ppm. |
| | | | LCMS (AM3): rt = 0.655 min, (498.1 |
| | | | [M + H]⁺), 100% purity. |
| | | | Purification Method 117 |

### Example 133

### 5-((3-(3-((3,5-Difluoro-4-(trifluoromethoxy)benzyl)amino)propanamido)propyl)amino)benzo[c][2,6]naphthyridin e-8-carboxamide

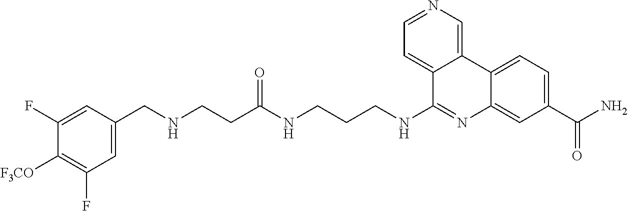 [SMILES: NC(=O)c1ccc2c(c1)nc(NCCCNC(=O)CCNCc1cc(F)c(OC(F)(F)F)c(F)c1)c1ccncc12]

A mixture of Intermediate C (80 mg, 218.33 μmol), NaOAc (71.64 mg, 873.32 μmol) and 3,5-difluoro-4-(trifluoromethoxy)benzaldehyde (49.36 mg, 218.33 μmol) in MeOH (3 mL) was stirred at 20° C. for 12 h, then NaBH(OAc)₃ (138.82 mg, 654.99 μmol) was added. The reaction mixture was stirred at 20° C. for 3 h. The reaction mixture was filtered and the filtrate was concentrated in vacuo. The crude product was purified (PM126) to afford EXAMPLE 133 (9.8 mg, 14.19 μmol, 6.5% yield, TFA salt) as a brown solid.

LCMS (AM3): rt=0.761 min, (577.3 [M+H]⁺), 100% purity.

¹H NMR (400 MHz, MeOH-d₄) δ: 10.07 (s, 1H), 8.93 (d, J=5.6 Hz, 1H), 8.76-8.73 (dd, 1H), 8.41 (s, 1H), 8.31 (d, J=5.6 Hz, 1H), 7.97-7.95 (d, 1H), 7.40 (d, J=8.1 Hz, 2H), 4.32 (s, 2H), 3.85 (t, J=6.6 Hz, 2H), 3.41-3.33 (m, 4H), 2.78 (t, J=6.6 Hz, 2H), 2.08-2.02 (quintet 2H) ppm.

The following examples in Table 6 were made with non-critical changes or substitutions to the exemplified procedure in EXAMPLE 133, that would be understood by one skilled in the art using Intermediate C and compounds of formula (Ic).

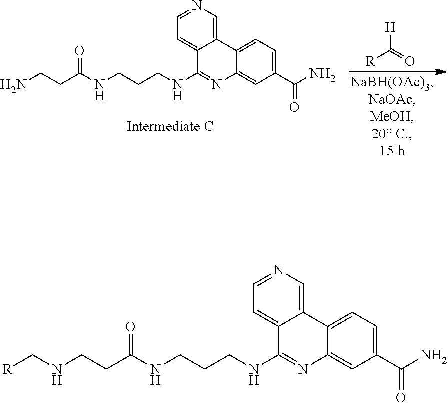

TABLE 6

| Example | Chemical | | |
|---|---|---|---|
| No. | IUPAC name | Compound (Ic) | Analytical |
| EXAMPLE 134 | 5-((3-(3-((3- Chloro-5- (cyanomethyl) benzyl)amino) propanamido) propyl)amino) benzo[c] [2,6]naph- thyridine-8- | 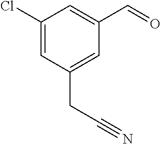 [SMILES: N#CCc1cc(Cl)cc(C=O)c1] | ¹H NMR (400 MHz, MeOH-d₄) δ: 10.06 (s, 1H), 8.95 (d, J = 5.6 Hz, 1H), 8.75 (d, J = 8.8 Hz, 1H), 8.45 (s, 1H), 8.36 (d, J = 5.6 Hz, 1H), 7.99 (dd, J = 1.6, 8.4 Hz, 1H), 7.53 (s, 1H), 7.49 (s, 1H), 7.46 (s, 1H), 4.28 (s, 2H), 3.95 (s, 2H), 3.87 (t, J = 6.6 Hz, 2H), 3.43-3.39 (t, 2H), 3.38- 3.34 (t, 2H), 2.77 (t, J = 6.8 Hz, 2H), 2.07 (quin, J = 6.4 Hz, 2H) ppm. |
| | carboxamide | 2-(3-chloro-5- | LCMS (AM3): rt = 0.703 min, (530.4 |
| | | formylphenyl) | [M + H]⁺), 100% purity. |
| | | acetonitrile | Purification Method 147 |
| | | (WO2022185041) | |

### Example 135

### 5-((2-(2-(2-((3-Chloro-4-(trifluoromethoxy)benzyl)amino)ethoxy)ethoxy)ethyl)amino)benzo[c][2,6]naphthyridin e-8-carboxamide

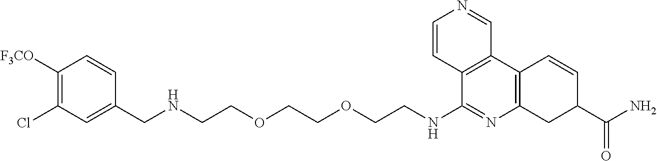 [SMILES: NC(=O)C1C=Cc2c(nc(NCCOCCOCCNCc3ccc(OC(F)(F)F)c(Cl)c3)c3ccncc23)C1]

A mixture of compound 1.187 (70 mg, 189.49 μmol) and 3-chloro-4-(trifluoromethoxy)benzaldehyde (CAS 83279-39-4, 42.55 mg, 189.49 μmol) in MeOH (1 mL) was stirred at 25° C. for 1 h and then NaBH(OAc)₃ (80.32 mg, 378.98 μmol) was added. The mixture was stirred at 25° C. for 11 h. The reaction mixture was concentrated in vacuo to give a residue which was purified (PM127) to afford EXAMPLE 135 (5.86 mg, 8.47 μmol, 4.5% yield, TFA salt) as a yellow oil.

LCMS (AM3): rt=0.754 min, (578.2 [M+H]⁺), 100% purity.

¹H NMR (400 MHz, MeOH-d₄) δ: 10.05 (s, 1H), 8.94 (d, J=5.6 Hz, 1H), 8.75 (d, J=8.8 Hz, 1H), 8.36 (d, J=1.6 Hz, 1H), 8.30 (d, J=5.6 Hz, 1H), 8.00 (dd, J=1.6, 8.4 Hz, 1H), 7.68 (d, J=2.0 Hz, 1H), 7.49-7.41 (m, 2H), 4.21 (s, 2H), 4.08-4.04 (t, 2H), 3.96-3.93 (t, 2H), 3.80-3.78 (m, 2H), 3.75-3.65 (m, 4H), 3.20-3.17 (t, 2H) ppm.

### Example 136

### 5-((2-(4-(2-((3-Chloro-4-(trifluoromethoxy)benzyl)amino)ethyl)-1H-1,2,3-triazol-1-yl)ethyl)amino)benzo[c][2,6]naphthyridine-8-carboxamide

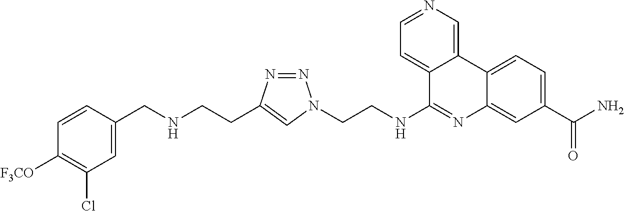 [SMILES: NC(=O)c1ccc2c(c1)nc(NCCn1cc(CCNCc3ccc(OC(F)(F)F)c(Cl)c3)nn1)c1ccncc12]

A mixture of Intermediate K (60 mg, 145.32 μmol, HCl salt), NaOAc (35.76 mg, 435.97 μmol) and 3-chloro-4-(trifluoromethoxy)benzaldehyde (CAS 83279-39-4, 32.63 mg, 145.32 μmol) in MeOH (2 mL) was stirred at 35° C. for 12.5 h, then NaBH(OAc)₃ (123.20 mg, 581.29 μmol) was added. The reaction mixture was stirred at 35° C. for 3 h. The mixture was concentrated in vacuo and the residue was purified (PM130) to afford EXAMPLE 136 (17.7 mg, 25.32 μmol, 17.4% yield, TFA salt) as a yellow solid.

LCMS (AM3): rt=0.774 min, (585.1 [M+H]⁺), 100% purity.

¹H NMR (400 MHz, MeOH-d₄) δ: 10.00 (s, 1H), 8.84 (d, J=5.6 Hz, 1H), 8.65 (d, J=8.4 Hz, 1H), 8.19 (d, J=1.6 Hz, 1H), 8.16 (d, J=5.6 Hz, 1H), 7.96 (s, 1H), 7.88 (dd, J=8.4, 1.6 Hz, 1H), 7.74 (s, 1H), 7.53 (s, 2H), 4.83 (t, J=5.6 Hz, 2H), 4.26 (t, J=5.6 Hz, 2H), 4.21 (s, 2H), 3.26-3.23 (t, 2H), 3.03-2.99 (t, 2H) ppm.

The following examples in Table 7 were made with non-critical changes or substitutions to the exemplified procedure in EXAMPLE 136, that would be understood by one skilled in the art using Intermediate K and compounds of formula (Ic).

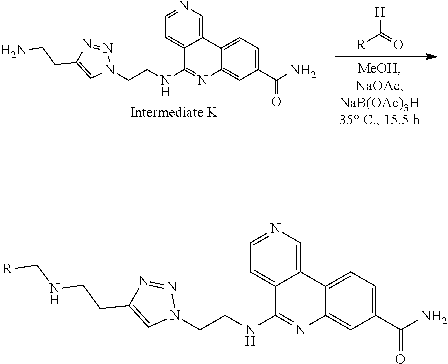

TABLE 7

| Example | Chemical | | |
|---|---|---|---|
| No. | IUPAC name | Compound (Ic) | Analytical |
| EXAMPLE 137 | 5-((2-(4-(2- (((6-Methyl-5- phenylpyridin- 2- yl)methyl)amino) ethyl)-1H- 1,2,3-triazol- 1- | 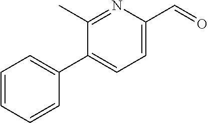 | ¹H NMR (400 MHz, MeOH-d₄) δ: 10.02 (s, 1H), 8.87 (d, J = 5.6 Hz, 1H), 8.67 (d, J = 8.4 Hz, 1H), 8.24-8.19 (m, 2H), 7.96 (s, 1H), 7.91 (dd, J = 1.8, 8.4 Hz, 1H), 7.66 (d, J = 7.8 Hz, 1H), 7.53-7.39 (m, 3H), 7.34-7.31 (m, 3H), 4.90-4.87 (m, 2H), 4.34 (s, 2H) 4.29 (t, J = 5.6 Hz, 2H), 3.29-3.27 (m, 2H), 3.09 (t, J = |
| | yl)ethyl)amino) | 6-methyl-5- | 7.2 Hz, 2H), 2.44 (s, 3H) ppm |
| | benzo[c][2,6] | phenylpicolin- | LCMS (AM3): rt = 0.757 min, (558.2 |
| | naphthyridine- | aldehyde 1.129 | [M + H]⁺), 97.8% purity. |
| | 8-carboxamide | | Purification Method 116 |
| EXAMPLE 138 | 5-((2-(4-(2- ((3- chlorobenzyl) amino)ethyl)- 1H-1,2,3- triazol-1- yl)ethyl)amino) | 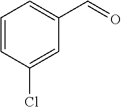 | ¹H NMR (400 MHz, MeOH-d₄) δ: 9.97 (s, 1H), 8.81 (d, J = 5.8 Hz, 1H), 8.64 (d, J = 8.4 Hz, 1H), 8.16 (d, J = 1.8 Hz, 1H), 8.09 (d, J = 5.5 Hz, 1H), 7.93 (s, 1H), 7.85 (dd, J = 1.9, 8.4 Hz, 1H), 7.50-7.35 (m, 4H), 4.24 (t, J = 5.5 Hz, 2H), 4.15 (s, 2H), 3.35-3.30 (m, 2H), |
| | benzo[c][2,6] | 3- | 3.20-3.13 (q, 2H), 3.02-2.97 (t, 2H) |
| | naphthyridine- | chlorobenzalde- | ppm |
| | 8- | hyde CAS 587- | LCMS (AM3): rt = 0.716 min, (501.2 |
| | carboxamide | 04-2 | [M + H]⁺), 100% purity. |
| | | | Purification Method 116 |
| EXAMPLE 139 | 5-((2-(4-(2- (((5-(Tri- fluoromethyl)- 1H-indol- 2- yl)methyl)amino) ethyl)-1H- | 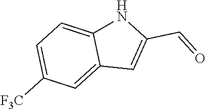 [SMILES: O=Cc1cc2cc(C(F)(F)F)ccc2[nH]1] | ¹H NMR (400 MHz, MeOH-d₄) δ: 9.96 (s, 1H), 8.81 (d, J = 5.6 Hz, 1H), 8.60 (d, J = 8.8 Hz, 1H), 8.16 (d, J = 1.6 Hz, 1H), 8.10 (d, J = 5.6 Hz, 1H), 7.94 (s, 1H), 7.90 (s, 1H), 7.83 (dd, J = 1.8, 8.4 Hz, 1H), 7.55 (d, J = 8.6 Hz, 1H), 7.44 (d, J = 10.1 Hz, 1H), 6.76 (s, 1H), |
| | 1,2,3-triazol-1- | 5- | 4.82-4.79 (m, 2H), 4.40 (s, 2H), 4.23 (t, |
| | yl)ethyl)amino) | (trifluoromethyl)- | J = 5.6 Hz, 2H), 3.24 (t, J = 8.0 Hz, 2H), |
| | benzo[c][2,6] | 1H-indole-2- | 3.02 (t, J = 7.2 Hz, 2H) ppm. |
| | naphthyridine- | carbaldehyde | LCMS (AM3): rt = 0.789 min, (574.2 |
| | 8- | CAS 1367793- | [M + H]⁺), 100% purity. |
| | carboxamide | 79-0 | Purification Method 44 |
| EXAMPLE 140 | 5-((2-(4-(2- ((3-chloro-5- fluorobenzyl) amino)ethyl)- | 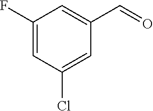 | ¹H NMR (400 MHz, MeOH-d₄) δ: 9.98 (s, 1H), 8.82 (d, J = 5.8 Hz, 1H), 8.65 (d, J = 8.5 Hz, 1H), 8.16 (d, J = 1.8 Hz, 1H), 8.11 (d, J = 5.6 Hz, 1H), 7.95 (s, |
| | 1H-1,2,3- | 3-chloro-5- | 1H), 7.86 (dd, J = 1.9, 8.4 Hz, 1H), |
| | triazol-1- | fluorobenzalde- | 7.37-7.32 (m, 2H), 7.22 (d, J = 9.3 Hz, |
| | yl)ethyl)amino) | hyde CAS 90390- | 1H), 4.84-4.81 (t, 2H), 4.24 (t, J = |
| | benzo[c][2,6] | 49-1 | 5.6 Hz, 2H), 4.17 (s, 2H), 3.19 (t, J = |
| | naphthyridine- | | 7.6 Hz, 2H), 3.00 (t, J = 7.7 Hz, 2H) ppm. |
| | 8- | | LCMS (AM3): rt = 0.722 min, (519.2 |
| | carboxamide | | [M + H]⁺), 94.7% purity. |
| | | | Purification Method 42 |
| EXAMPLE 141 | 5-((2-(4-(2- ((3,5- dichlorobenzyl) amino)ethyl)- 1H-1,2,3- triazol-1- yl)ethyl)amino) | 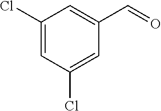 | ¹H NMR (400 MHz, MeOH-d₄) δ: 9.98 (s, 1H), 8.82 (d, J = 5.8 Hz, 1H), 8.65 (d, J = 8.5 Hz, 1H), 8.16 (d, J = 1.8 Hz, 1H), 8.11 (d, J = 5.3 Hz, 1H), 7.95 (s, 1H), 7.86 (dd, J = 1.9, 8.4 Hz, 1H), 7.57 (t, J = 1.8 Hz, 1H), 7.47 (d, J = 1.9 Hz, 2H), 4.84-4.80 (m, 2H), 4.24 (t, J = |
| | benzo[c][2,6] | 3,5- | 5.6 Hz, 2H), 4.15 (s, 2H), 3.20-3.17 (t, |
| | naphthyridine- | dichlorobenzalde- | 2H), 3.02-2.98 (t, 2H) ppm. |
| | 8- | hyde CAS | LCMS (AM3): rt = 0.741 min, (535.1 |
| | carboxamide | 10203-08-4 | [M + H]⁺), 97.5% purity. |
| | | | Purification Method 157 |
| EXAMPLE 142 | 5-((2-(4-(2- (((6-Chloro- 1H-indol-2- yl)methyl)amino) ethyl)-1H- 1,2,3-triazol-1- | 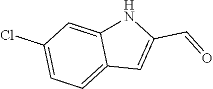 [SMILES: O=Cc1cc2ccc(Cl)cc2[nH]1] | ¹H NMR (400 MHz, MeOH-d₄) δ: 9.95 (s, 1H), 8.80 (d, J = 5.7 Hz, 1H), 8.59 (d, J = 8.4 Hz, 1H), 8.15 (d, J = 1.8 Hz, 1H), 8.08 (d, J = 5.7 Hz, 1H), 7.93 (s, 1H), 7.82 (dd, J = 1.8, 8.4 Hz, 1H), 7.51 (d, J = 8.6 Hz, 1H), 7.41 (s, 1H), 7.05 |
| | yl)ethyl)amino) | 6-chloro-1H- | (dd, J = 1.9, 8.5 Hz, 1H), 6.61 (s, 1H), |
| | benzo[c][2,6] | indole-2- | 4.82-4.78 (m, 2H), 4.34 (s, 2H), 4.23 |
| | naphthyridine- | carbaldehyde | (t, J = 5.6 Hz, 2H), 3.22-3.18 (t, 2H), |
| | 8- | CAS 53590-59-3 | 3.02-2.97 (t, 2H) ppm. |
| | carboxamide | | LCMS (AM3): rt = 0.761 min, (540.1 |
| | | | [M + H]⁺), 100% purity. |
| | | | Purification Method 147 |
| EXAMPLE 143 | 5-((2-(4-(2- ((3-Chloro-4- cyclobutoxy- benzyl)amino) ethyl)-1H- 1,2,3-triazol- 1- yl)ethyl)amino) benzo[c][2,6] | 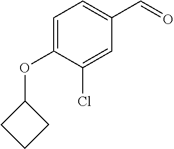 [SMILES: O=Cc1ccc(OC2CCC2)c(Cl)c1] | ¹H NMR (400 MHz, MeOH-d₄) δ: 9.99 (s, 1H), 8.83 (d, J = 5.7 Hz, 1H), 8.65 (d, J = 8.4 Hz, 1H), 8.19 (d, J = 1.8 Hz, 1H), 8.13 (d, J = 5.7 Hz, 1H), 7.94 (s, 1H), 7.87 (dd, J = 1.8, 8.4 Hz, 1H), 7.48 (d, J = 2.2 Hz, 1H), 7.29 (dd, J = 2.2, 8.4 Hz, 1H), 6.94 (d, J = 8.6 Hz, 1H), 4.88-4.86 (m, 1H), 4.79-4.73 (quin, 2H), 4.25 (t, J = 5.7 Hz, 2H), 4.07 (s, |
| | naphthyridine- | 3-chloro-4- | 2H), 3.16 (t, J = 8.0 Hz, 2H), 2.98 (t, J = |
| | 8- | cyclobutoxy- | 8.0 Hz, 2H), 2.54-2.45 (m, 2H), |
| | carboxamide | benzaldehyde | 2.22-2.12 (quin, 2H), 1.94-1.71 (m, |
| | | (WO2022185041) | 2H) ppm. |
| | | | LCMS (AM3): rt = 0.776 min, (571.2 |
| | | | [M + H]⁺), 100% purity. |
| | | | Purification Method 172 |
| EXAMPLE 144 | 5-((2-(4-(2- ((3-Chloro-4- cyclopropyl- benzyl)amino) ethyl)-1H- 1,2,3-triazol- 1- | 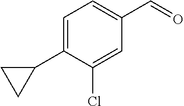 [SMILES: O=Cc1ccc(C2CC2)c(Cl)c1] | ¹H NMR (400 MHz, MeOH-d₄) δ: 9.99 (s, 1H), 8.83 (d, J = 5.7 Hz, 1H), 8.64 (d, J = 8.4 Hz, 1H), 8.18 (d, J = 1.7 Hz, 1H), 8.12 (d, J = 5.6 Hz, 1H), 7.94 (s, 1H), 7.87 (dd, J = 1.8, 8.4 Hz, 1H), 7.48 (d, J = 1.8 Hz, 1H), 7.27 (dd, J = 1.7, 8.1 Hz, 1H), 7.04 (d, J = 8.1 Hz, 1H), |
| | yl)ethyl)amino) | 3-Chloro-4- | 4.83-4.81 (m, 2H), 4.25 (t, J = 5.6 Hz, |
| | benzo[c][2,6] | cyclopropylbenz- | 2H), 4.10 (s, 2H), 3.16 (t, J = 4 Hz, 2H), |
| | naphthyridine- | aldehyde | 2.99 (t, J = 7.6 Hz, 2H), 2.27-2.18 |
| | 8- | (WO2022185041) | (quin, 1H), 1.09-1.04 (q, 2H), 0.72- |
| | carboxamide | | 0.68 (q, 2H) ppm. |
| | | | LCMS (AM3): rt = 0.762 min, (541.2 |
| | | | [M + H]⁺), 100% purity. |
| | | | Purification Method 173 |
| EXAMPLE 145 | 5-((2-(4-(2- ((3-fluoro-4- (trifluorometh- oxy)benzyl) amino)ethyl)- 1H-1,2,3- triazol-1- | 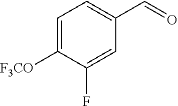 [SMILES: O=Cc1ccc(OC(F)(F)F)c(F)c1] | ¹H NMR (400 MHz, MeOH-d₄) δ: 9.97 (s, 1H), 8.81 (d, J = 5.7 Hz, 1H), 8.64 (d, J = 8.4 Hz, 1H), 8.15 (d, J = 1.8 Hz, 1H), 8.09 (d, J = 5.5 Hz, 1H), 7.95 (s, 1H), 7.85 (dd, J = 1.8, 8.3 Hz, 1H), 7.56-7.47 (m, 2H), 7.40-7.35 (m, 1H), 4.81-4.80 (m, 2H), 4.26-4.20 (m, 4H), |
| | yl)ethyl)amino) | 3-fluoro-4- | 3.23-3.20 (t, 2H), 3.02-2.98 (t, 2H) |
| | benzo[c][2,6] | (trifluoromethoxy) | ppm. |
| | naphthyridine- | benzaldehyde | LCMS (AM3): rt = 0.756 min, (569.2 |
| | 8- | CAS 473917-15- | [M + H]⁺), 100% purity. |
| | carboxamide | 6 | Purification Method 173 |
| EXAMPLE 146 | 5-((2-(4-(2- (((2-Chloro- 2′-(2- hydroxyethoxy)- [1,1′- biphenyl]-4- yl)methyl)amino) ethyl)-1H- 1,2,3-triazol-1- yl)ethyl)amino) | 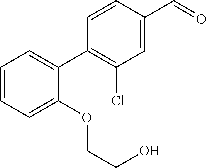 [SMILES: O=Cc1ccc(-c2ccccc2OCCO)c(Cl)c1] | ¹H NMR (400 MHz, MeOH-d₄) δ: 9.99 (s, 1H), 8.83 (d, J = 5.6 Hz, 1H), 8.65 (d, J = 8.4 Hz, 1H), 8.19 (d, J = 1.6 Hz, 1H), 8.14 (d, J = 5.6 Hz, 1H), 7.96 (s, 1H), 7.88 (dd, J = 8.4, 1.6 Hz, 1H), 7.59 (s, 1H), 7.43-7.35 (m, 3H), 7.14-7.08 (m, 2H), 7.06-7.02 (t, 1H), 4.83-4.81 (m, 2H), 4.26 (t, J = 5.6 Hz, 2H), 4.19 (s, 2H), 4.04 (t, J = 4.8 Hz, 2H), 3.69 (t, J = 5.2 Hz, 2H), 3.22 (t, J = 5.2 Hz, 2H), |
| | benzo[c][2,6] | 2-chloro-2′-(2- | 3.03 (t, J = 5.2 Hz, 2H) ppm. |
| | naphthyridine- | hydroxyethoxy)- | LCMS (AM3): rt = 0.772 min, (637.1 |
| | 8- | [1,1′-biphenyl]- | [M + H]⁺), 100% purity. |
| | carboxamide | 4-carbaldehyde | Purification Method 117 |
| | | 1.241 | |
| EXAMPLE 147 | 5-((2-(4-(2- (((5-Chloro-6- phenylpyridin- 3- yl)methyl)amino) ethyl)-1H- 1,2,3-triazol- 1- | 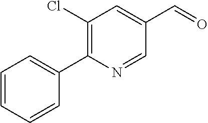 [SMILES: O=Cc1cnc(-c2ccccc2)c(Cl)c1] | ¹H NMR (400 MHz, MeOH-d₄) δ: 9.99 (s, 1H), 8.84 (d, J = 5.6 Hz, 1H), 8.66-8.62 (d, 1H), 8.62 (d, 1H), 8.20-8.19 (d, 1H), 8.15-8.13 (d, 1H), 8.12-8.11 (d, 1H), 7.98 (s, 1H), 7.88 (dd, J = 1.7, 8.6 Hz, 1H), 7.71-7.65 (m, 2H), 7.53-7.48 (m, 3H), 4.83-4.81 (m, 2H), 4.31 (s, 2H), 4.26 (t, J = 5.5 Hz, |
| | yl)ethyl)amino) | 5-Chloro-6- | 2H), 3.30-3.28 (m, 2H), 3.07-3.03 (t, |
| | benzo[c][2,6] | phenylnicotin- | 2H) ppm. |
| | naphthyridine- | aldehyde CAS | LCMS (AM3): rt = 0.879 min, (578.2 |
| | 8- | 1227582-64-0 | [M + H]⁺), 95.5% purity |
| | carboxamide | | Purification Method 126 |
| EXAMPLE 148 | 5-((2-(4-(2- (((1-Methyl-5- (trifluoromethyl)- 1H-indol- 2- yl)methyl)amino) ethyl)-1H- | 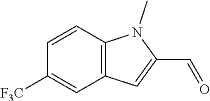 [SMILES: Cn1c(C=O)cc2cc(C(F)(F)F)ccc21] | ¹H NMR (400 MHz, MeOH-d₄) δ: 9.94 (s, 1H), 8.80 (d, J = 5.6 Hz, 1H), 8.59 (d, J = 8.4 Hz, 1H), 8.15 (d, J = 1.6 Hz, 1H), 8.09 (d, J = 5.6 Hz, 1H), 7.97 (s, 1H), 7.90 (s, 1H), 7.81 (dd, J = 1.8, 8.4 Hz, 1H), 7.59 (dd, J = 1.6, 8.7 Hz, 1H), 7.50 (dd, J = 1.6, 8.7 Hz, 1H), 6.81 |
| | 1,2,3-triazol-1- | 1-Methyl-5- | (s, 1H), 4.81-4.79 (m, 2H), 4.50 (s, 2H), |
| | yl)ethyl)amino) | (trifluoromethyl)- | 4.24 (t, J = 5.6 Hz, 2H), 3.86 (s, |
| | benzo[c][2,6] | 1H-indole-2- | 3H), 3.30-3.28 (m, 2H), 3.05 (t, J = |
| | naphthyridine- | carbaldehyde | 7.6 Hz, 2H) ppm. |
| | 8- | CAS 2090356- | LCMS (AM3): rt = 0.785 min, (588.2 |
| | carboxamide | | [M + H]⁺), 100% purity. |
| | | | Purification Method 156 |

### Example 149

### 5-((3-(3-((3-(2-Hydroxyethoxy)benzyl)amino)propanamido)propyl)amino)benzo[c][2,6]naphthyridine-8-carboxylic acid

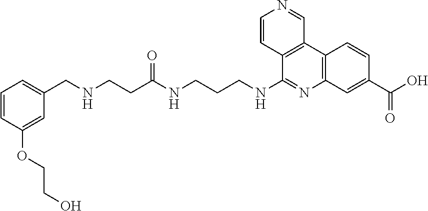 [SMILES: O=C(CCNCc1cccc(OCCO)c1)NCCCNc1nc2cc(C(=O)O)ccc2c2cnccc12]

A mixture of compound 1.540 (70 mg, 173.33 μmol, HCl salt), 3-(2-hydroxyethoxy)benzaldehyde (28.80 mg, 173.33 μmol) and NaOAc (56.87 mg, 693.31 μmol) in MeOH (3 mL) was stirred at 20° C. for 12 h before NaBH(OAc)₃ (110.21 mg, 519.98 μmol) was added. The reaction mixture was stirred at 20° C. for 3 h. The reaction mixture was filtered and the filtrate was purified (PM174) to afford EXAMPLE 149 (41.63 mg, 80.43 μmol, 46.4% yield) as a yellow solid.

LCMS (AM7): rt=0.641 min, (518.4 [M+H]⁺), 96.4% purity.

¹H NMR (400 MHz, DMSO-d₆) δ: 10.03 (s, 1H), 8.85 (d, J=5.6 Hz, 1H), 8.65 (d, J=8.4 Hz, 1H), 8.24 (d, J=5.6 Hz, 1H), 8.20-8.16 (m, 2H), 7.94 (t, J=4.8 Hz, 1H), 7.84 (dd, J=8.4, 1.6 Hz, 1H), 7.19 (t, J=8.0 Hz, 1H), 7.00 (s, 1H), 6.92 (d, J=7.6 Hz, 1H), 6.79 (dd, J=8.0, 2.0 Hz, 1H), 3.94 (t, J=4.8 Hz, 2H), 3.80 (s, 2H), 3.67 (t, J=4.8 Hz, 2H), 3.62 (t, J=5.2 Hz, 2H), 3.19 (q, J=6.0 Hz, 2H), 2.82 (t, J=7.2 Hz, 2H), 2.42 (t, J=7.2 Hz, 2H), 1.88-1.81 (quin, 2H) ppm.

### Example 150

### 5-((3-(3-((3-Chloro-4-cyclobutoxybenzyl)amino)propanamido)propyl)amino)benzo[c][2,6]naphthyridine-8-carboxylic acid

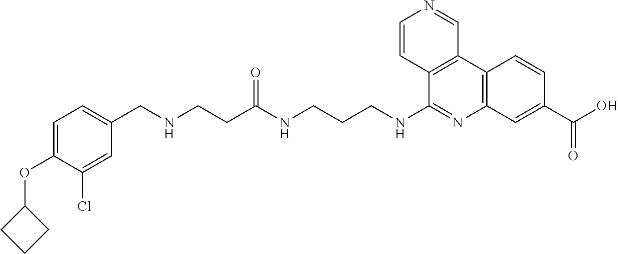 [SMILES: O=C(CCNCc1ccc(OC2CCC2)c(Cl)c1)NCCCNc1nc2cc(C(=O)O)ccc2c2cnccc12]

A mixture of compound 1.540 (40 mg, 99.04 μmol), NaOAc (32.50 mg, 396.18 μmol) and 3-chloro-4-cyclobutoxybenzaldehyde (ref: WO2022185041, 20.86 mg, 99.04 μmol) in MeOH (3 mL) was stirred at 20° C. for 12.5 h, then NaBH(OAc)₃ (62.97 mg, 297.13 μmol) was added. The reaction mixture was stirred at 20° C. for 3 h. The reaction mixture was filtered and the filtrate was concentrated in vacuo. The residue was purified (PM136) to afford EXAMPLE 150 (25.26 mg, 34.26 μmol, 34.6% yield, TFA salt) as a yellow solid.

LCMS (AM3): rt=0.764 min, (562.5 [M+H]⁺), 91.7% purity.

¹H NMR (400 MHz, DMSO-d₆) δ: 10.10 (s, 1H), 8.94 (d, J=5.6 Hz, 1H), 8.80-8.73 (m, 3H), 8.35 (d, J=5.6 Hz, 1H), 8.27-8.18 (m, 2H), 7.88 (d, J=7.6 Hz, 1H), 7.58 (d, J=2.4 Hz, 1H), 7.36 (dd, J=8.4, 2.0 Hz, 1H), 7.02 (d, J=8.4 Hz, 1H), 4.81-4.73 (quin, 1H), 4.10-4.06 (m, 2H), 3.72-3.66 (m, 2H), 3.26-3.21 (q, 2H), 3.13-3.03 (m, 2H), 2.54-2.41 (m, 4H), 2.09-1.99 (m, 2H), 1.94-1.86 (quin, 2H), 1.84-1.76 (m, 1H), 1.70-1.60 (m, 1H) ppm.

### Example 151

### 5-((3-(3-((3-Chloro-4-cyclobutoxybenzyl)amino)-N-methylpropanamido)propyl)amino)benzo[c][2,6]naphthyridine-8-carboxamide

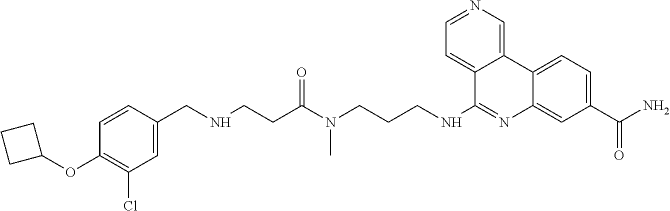 [SMILES: CN(CCCNc1nc2cc(C(N)=O)ccc2c2cnccc12)C(=O)CCNCc1ccc(OC2CCC2)c(Cl)c1]

A solution of compound 1.347 (70 mg, 167.90 μmol, HCl salt), 3-chloro-4-cyclobutoxybenzaldehyde (ref: WO2022185041, 35.37 mg, 167.90 μmol) and DIPEA (43.40 mg, 335.81 μmol) in MeOH (10 mL) was stirred at 30° C. for 3.5 h, then NaBH₃CN (31.65 mg, 503.71 μmol) was added. The reaction mixture was stirred at 30° C. for 3 h. The reaction mixture was filtered and the filtrate was concentrated in vacuo. The residue was purified (PM175) to afford EXAMPLE 151 (33.18 mg, 57.69 μmol, 34.4% yield) as a yellow oil.

LCMS (AM3): rt=0.897 min, (575.4 [M+H]⁺), 100% purity.

¹H NMR (400 MHz, DMSO-d₆) δ: 10.01 (s, 1H), 8.84 (d, J=5.6 Hz, 1H), 8.64 (d, J=8.4 Hz, 1H), 8.21 (d, J=5.2 Hz, 1H), 8.15 (d, J=1.6 Hz, 1H), 7.81 (dd, J=1.8, 8.4 Hz, 1H), 7.70-7.62 (br s, 1H), 7.33-7.26 (br s, 1H), 7.20-7.10 (br s, 1H), 6.82 (d, J=8.2 Hz, 1H), 4.68 (quin, J=6.8 Hz, 1H), 3.69-3.54 (m, 4H), 3.48 (t, J=7.2 Hz, 2H), 3.02 (s, 3H), 2.76-2.69 (br m, 2H), 2.48-2.46 (m, 2H), 2.45-2.35 (m, 2H), 2.12-1.92 (m, 4H), 1.85-1.75 (m, 1H), 1.70-1.60 (quin, 1H) ppm.

### Example 152

### 5-((3-(3-((3-Chloro-4-(trifluoromethoxy)benzyl)amino)propanamido)propyl)amino)benzo[c][2,6]naphthyridin e-8-carboxylic acid

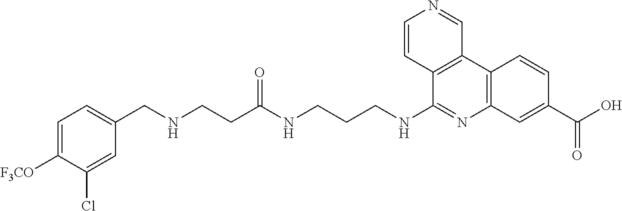 [SMILES: O=C(CCNCc1ccc(OC(F)(F)F)c(Cl)c1)NCCCNc1nc2cc(C(=O)O)ccc2c2cnccc12]

To a solution of compound 1.261 (100 mg, 0.16 mmol, HCl salt) in THF (5 mL), H₂O (0.5 mL) and MeOH (0.5 mL) was added LiOH·H₂O (66 mg, 1.60 mmol) at 25° C. The mixture was stirred at 25° C. for 1 h. The mixture was concentrated in vacuo to give a residue which was purified (PM176) to afford EXAMPLE 152 (93 mg, 0.13 mmol, 83.2% yield, TFA salt) as a yellow solid.

LCMS (AM3): rt=0.656 min, (576.2 [M+H]⁺), 98.5% purity.

¹H NMR (400 MHz, DMSO-d₆) δ: 10.11 (s, 1H), 8.96-8.84 (m, 3H), 8.79 (d, J=8.4 Hz, 1H), 8.36 (d, J=5.2 Hz, 1H), 8.31-8.24 (m, 2H), 7.90-7.86 (d, 1H), 7.85-7.84 (d, 1H), 7.67 (dd, J=8.4, 1.2 Hz, 1H), 7.59 (dd, J=8.4, 1.2 Hz, 1H) 4.21 (s, 2H), 3.75-3.65 (q, J=5.2 Hz, 2H), 3.25 (q, J=6.4 Hz, 2H), 3.19-3.09 (m, 2H), 2.57-2.53 (m, 2H), 1.94-1.86 (quin, 2H) ppm.

### Example 153

### 5-((3-(3-((3-Chloro-4-(trifluoromethoxy)benzyl)amino)propoxy)propyl)amino)benzo[c][2,6]naphthyridine-8-carboxylic acid

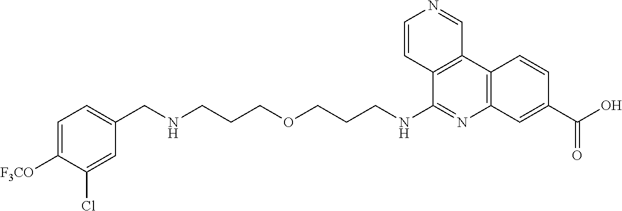 [SMILES: O=C(O)c1ccc2c(c1)nc(NCCCOCCCNCc1ccc(OC(F)(F)F)c(Cl)c1)c1ccncc12]

To a mixture of compound 1.541 (400 mg, 693.25 μmol) in MeOH (5 mL), THF (5 mL) and H₂O (5 mL) was added LiOH·H₂O (58.18 mg, 1.39 mmol). The reaction mixture was stirred at 20° C. for 12 h. The reaction mixture was concentrated under and purified (PM94) to afford EXAMPLE 153 (58.58 mg, 85.97 μmol, 12.4% yield, TFA salt) as a yellow solid.

LCMS (AM3): rt=0.776 min, (563.2 [M+H]⁺), 99.3% purity.

¹H NMR (400 MHz, MeOH-d₄) δ: 10.07 (s, 1H), 8.96 (d, J=5.6 Hz, 1H), 8.77 (d, J=8.4 Hz, 1H), 8.56 (d, J=1.6 Hz, 1H), 8.37 (d, J=6.0 Hz, 1H), 8.15 (dd, J=8.4, 1.6 Hz, 1H), 7.74 (d, J=2.0 Hz, 1H), 7.55-7.47 (q, 2H), 4.25 (s, 2H), 3.89 (t, J=7.2 Hz, 2H), 3.68 (t, J=6.0 Hz, 2H), 3.63 (t, J=5.6 Hz, 2H), 3.22 (t, J=7.2 Hz, 2H), 2.19-2.12 (quin, 2H), 2.08-2.00 (quin, 2H) ppm.

### Example 154

### 5-((3-((3-((3-Chloro-4-(trifluoromethoxy)benzyl)amino)propyl)amino)-3-oxopropyl)amino)benzo[c][2,6]naphthyridine-8-carboxylic acid

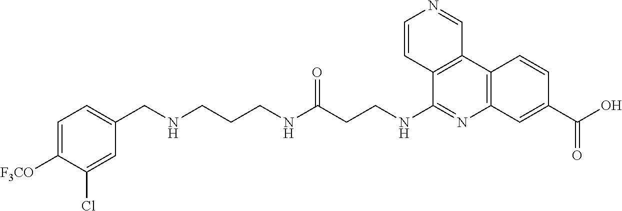 [SMILES: O=C(CCNc1nc2cc(C(=O)O)ccc2c2cnccc12)NCCCNCc1ccc(OC(F)(F)F)c(Cl)c1]

To a mixture of compound 1.544 (50 mg, 84.75 μmol) in MeOH (3 mL), THF (3 mL) and H₂O (3 mL) was added LiOH·H₂O (7.11 mg, 169.49 μmol) at 20° C. The reaction mixture was stirred at 20° C. for 12 h. The reaction mixture was filtered and the filtrate was purified (PM94) to afford EXAMPLE 154 (28.99 mg, 42.02 μmol, 49.6% yield, 100% purity, TFA salt) as a yellow solid.

LCMS (AM3): rt=0.759 min, (576.4 [M+H]⁺), 98.6% purity.

¹H NMR (400 MHz, MeOH-d₄) δ: 10.05 (s, 1H), 8.94 (d, J=5.6 Hz, 1H), 8.75 (d, J=8.8 Hz, 1H), 8.50 (d, J=1.2 Hz, 1H), 8.30 (d, J=5.6 Hz, 1H), 8.13 (dd, J=8.4, 1.6 Hz, 1H), 7.73 (d, J=2.0 Hz, 1H), 7.53-7.47 (m, 2H), 4.17 (s, 2H), 4.09 (t, J=6.0 Hz, 2H), 3.37 (t, J=6.4 Hz, 2H), 3.10 (t, J=7.2 Hz, 2H), 2.89 (t, J=6.0 Hz, 2H), 1.96-1.89 (quin, 2H) ppm.

### Example 155

### 5-((4-(2-((3-Chloro-4-(trifluoromethoxy)benzyl)amino)ethoxy)butyl)amino)benzo[c][2,6]naphthyridine-8-carboxylic acid

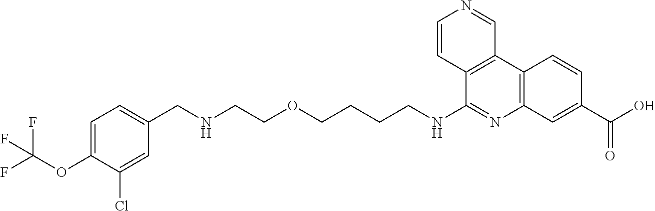 [SMILES: O=C(O)c1ccc2c(c1)nc(NCCCCOCCNCc1ccc(OC(F)(F)F)c(Cl)c1)c1ccncc12]

To a solution of compound 1.431 (80 mg, 130.41 μmol) in THF (3 mL), H₂O (3 mL) and MeOH (3 mL) was added LiOH·H₂O (27.36 mg, 652.05 μmol) at 20° C. The resulting mixture was stirred at 20° C. for 4 h. The mixture was concentrated in vacuo to afford the residue which was purified (PM173) to afford EXAMPLE 155 (43.01 mg, 63.53 μmol, 48.7% yield, TFA salt) as a yellow gum.

LCMS (AM3): rt=0.772 min, (563.2 [M+H]⁺), 100% purity.

¹H NMR (400 MHz, MeOH-d₄) δ: 10.05 (s, 1H), 8.93 (d, J=5.6 Hz, 1H), 8.74 (d, J=8.4 Hz, 1H), 8.52 (s, 1H), 8.33 (d, J=5.6 Hz, 1H), 8.11 (d, J=6.8 Hz, 1H), 7.69 (s, 1H), 7.47 (s, 2H), 4.25 (s, 2H), 3.82 (t, J=7.2 Hz, 2H), 3.78 (t, J=4.8 Hz, 2H), 3.65 (t, J=6.4 Hz, 2H), 3.26 (t, J=4.8 Hz, 2H), 2.01-1.93 (quin, 2H), 1.88-1.80 (quin, 2H) ppm.

### Example 156

### 5-((4-((2-((3-Chloro-4-(trifluoromethoxy)benzyl)amino)ethyl)amino)-4-oxobutyl)amino)benzo[c][2,6]naphthyridine-8-carboxylic acid

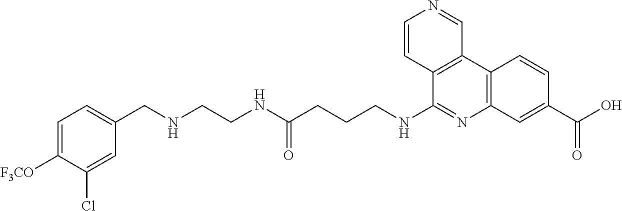 [SMILES: O=C(CCCNc1nc2cc(C(=O)O)ccc2c2cnccc12)NCCNCc1ccc(OC(F)(F)F)c(Cl)c1]

To a solution of compound 1.659 (40 mg, 67.80 μmol) in THF (1 mL), H₂O (1 mL) and MeOH (0.5 mL) was added LiOH·H₂O (8.54 mg, 203.39 μmol) at 25° C. The resulting mixture was stirred at 25° C. for 1 h. The mixture was concentrated in vacuo and purified (PM124) to afford EXAMPLE 156 (12.73 mg, 30.2% yield, FA salt) as a yellow solid.

LCMS (AM3): rt=0.737 min, (576.1 [M+H]⁺), 100% purity.

¹H NMR (400 MHz, MeOH-d₄) δ: 9.81 (s, 1H), 8.73 (d, J=5.6 Hz, 1H), 8.46 (d, J=8.4 Hz, 1H), 8.37 (br s, 1H), 8.22 (d, J=1.6 Hz, 1H), 8.04 (d, J=5.6 Hz, 1H), 7.90 (dd, J=8.4, 1.6 Hz, 1H), 7.70 (d, J=2.0 Hz, 1H), 7.51-7.41 (qd, 2H), 4.17 (s, 2H), 3.68 (t, J=6.8 Hz, 2H), 3.57 (t, J=5.6 Hz, 2H), 3.15 (t, J=6.0 Hz, 2H), 2.42 (t, J=6.8 Hz, 2H), 2.11-2.02 (m, 2H) ppm.

### Example 157

### 5-((2-(4-((3-Chloro-4-(trifluoromethoxy)benzyl)amino)butanamido)ethyl)amino)benzo[c][2,6]naphthyridine-8-carboxylic acid

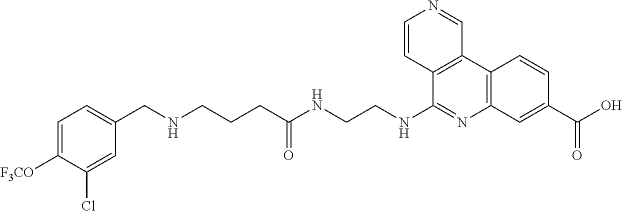 [SMILES: O=C(CCCNCc1ccc(OC(F)(F)F)c(Cl)c1)NCCNc1nc2cc(C(=O)O)ccc2c2cnccc12]

To a solution of compound 1.569 (50 mg, 84.75 μmol) in THF (1 mL), MeOH (1 mL) and H₂O (1 mL) was added LiOH·H₂O (7.11 mg, 169.49 μmol) at 20° C. The reaction mixture was stirred at 20° C. for 1 h. The reaction mixture was acidified with aq. 1 N HCl to pH 5 and then concentrated in vacuo. The residue was purified (PM177) to afford EXAMPLE 157 (14.36 mg, 24.93 μmol, 29.4% yield) as an off-white solid.

LCMS (AM3): rt=0.747 min, (576.1 [M+H]⁺), 99.6% purity.

¹H NMR (400 MHz, DMSO-d₆) δ: 10.05 (s, 1H), 8.86 (d, J=5.6 Hz, 1H), 8.69 (d, J=8.4 Hz, 1H), 8.21 (d, J=5.6 Hz, 1H), 8.17 (d, J=1.6 Hz, 1H), 8.09 (t, J=5.6 Hz, 1H), 8.04 (t, J=5.2 Hz, 1H), 7.83 (dd, J=8.4, 1.6 Hz, 1H), 7.59 (d, J=2.0 Hz, 1H), 7.46-7.44 (m, 1H), 7.39-7.36 (m, 1H), 3.65 (s, 2H), 3.43 (t, J=6.0 Hz, 2H), 2.53-2.51 (m, 2H), 2.44 (t, J=7.2 Hz, 2H), 2.13 (t, J=7.2 Hz, 2H), 1.66 (quint, J=6.8 Hz, 2H) ppm.

### Example 158

### 5-(2-(4-((3-Chloro-4-(trifluoromethoxy)benzyl)amino)butoxy)ethyl)-4-oxo-4,5-dihydro-3H-pyrazolo[3,4-c]quinoline-7-carboxylic acid

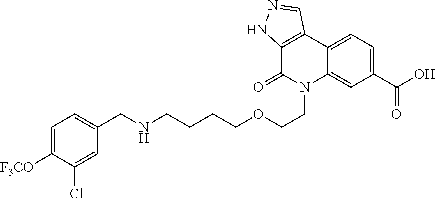 [SMILES: O=C(O)c1ccc2c3cn[nH]c3c(=O)n(CCOCCCCNCc3ccc(OC(F)(F)F)c(Cl)c3)c2c1]

To a solution of compound 1.816 (50 mg, 0.09 mmol) in THF (2 mL) and H₂O (2 mL) was added LiOH·H₂O (20 mg, 0.84 mmol) at RT. The mixture was stirred at RT for 1 h. The reaction mixture was concentrated in vacuo and the residue was purified (PM178) to afford EXAMPLE 158 (29.65 mg, 0.05 mmol, 60.8% yield) as a white solid.

LCMS (AM3): rt=0.822 min, (553.3 [M+H]⁺), 99.7% purity.

¹H NMR (400 MHz, MeOH-d₄) δ: 8.49-8.45 (m, 2H), 8.04-8.01 (m, 1H), 7.92 (d, J=8.0 Hz, 1H), 7.87 (s, 1H), 7.67 (d, J=8.4 Hz, 1H), 7.51 (d, J=8.4 Hz, 1H), 4.68-4.67 (m, 2H), 4.18 (s, 2H), 3.88-3.87 (m, 2H), 3.48 (t, J=5.2 Hz, 2H), 2.95 (t, J=8.0 Hz, 2H), 1.87-1.79 (quin, 2H), 1.58-1.52 (quin, 2H) ppm.

### Example 159

### 5-(4-(3-((3-Chloro-4-(trifluoromethoxy)benzyl)amino)propoxy)piperidin-1-yl)benzo[c][2,6]naphthyridine-8-carboxylic acid

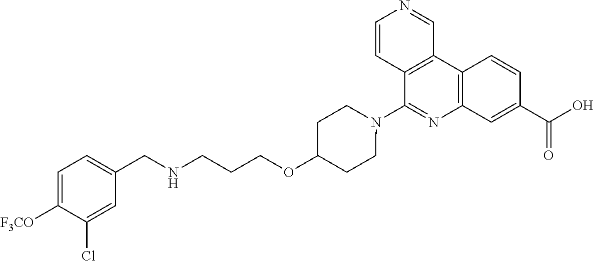 [SMILES: O=C(O)c1ccc2c(c1)nc(N1CCC(OCCCNCc3ccc(OC(F)(F)F)c(Cl)c3)CC1)c1ccncc12]

To a mixture of compound 1.603 (100 mg, 165.83 μmol) in THF (9 mL), MeOH (2 mL) and H₂O (2 mL) was added LiOH·H₂O (41.75 mg, 994.97 μmol) at 35° C. The mixture was stirred at 35° C. for 12 h. The mixture was adjusted to pH 5 by adding aq. HCl solution (1 N) and concentrated in vacuo. The residue was purified (PM179) to afford EXAMPLE 159 (63.37 mg, 106.51 μmol, 64.2% yield) as an off-white solid.

LCMS (AM3): rt=0.850 min, (589.2 [M+H]⁺), 99.5% purity.

¹H NMR (400 MHz, DMSO-d₆) δ: 10.18 (s, 1H), 8.90 (d, J=5.6 Hz, 2H), 8.33 (d, J=1.6 Hz, 1H), 8.16 (s, 1H), 8.03 (dd, J=8.4, 1.6 Hz, 1H), 7.97 (d, J=5.6 Hz, 1H), 7.67 (d, J=2.0 Hz, 1H), 7.52-7.43 (m, 2H), 3.79 (s, 2H), 3.77-3.69 (m, 2H), 3.63-3.53 (m, 3H), 3.24-3.20 (m, 2H), 2.63 (t, J=8.2 Hz, 2H), 2.08-2.02 (m, 2H), 1.79-1.69 (m, 4H) ppm.

### Example 160

### 5-(4-(4-((3-Chloro-4-(trifluoromethoxy)benzyl)amino)butyl)piperazin-1-yl)benzo[c][2,6]naphthyridine-8-carboxylic acid

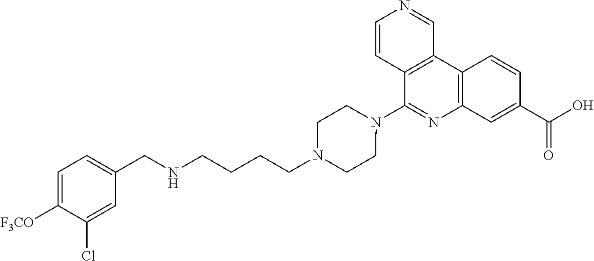 [SMILES: O=C(O)c1ccc2c(c1)nc(N1CCN(CCCCNCc3ccc(OC(F)(F)F)c(Cl)c3)CC1)c1ccncc12]

To a solution of compound 1.620 (100 mg, 154.30 μmol, FA salt) in H₂O (1 mL) and THF (1 mL) was added LiOH·H₂O (20 mg, 476.80 μmol) at 20° C. The resulting mixture was stirred at 20° C. for 1 h. The mixture was concentrated in vacuo and the residue was purified (PM124) to afford EXAMPLE 160 (38.99 mg, 61.08 μmol, 39.6% yield, FA salt) as a yellow solid.

LCMS (AM3): rt=0.763 min, (588.6 [M+H]⁺), 99.3% purity.

¹H NMR (400 MHz, MeOH-d₄) δ: 9.83 (s, 1H), 8.66 (d, J=5.6 Hz, 1H), 8.58 (d, J=8.4 Hz, 1H), 8.46 (s, 2H), 8.16 (dd, J=8.4, 1.2 Hz, 1H), 7.83 (d, J=2.0 Hz, 1H), 7.70-7.67 (m, 1H), 7.61 (dd, J=8.4, 2.0 Hz, 1H), 7.50 (dd, J=8.4, 1.2 Hz, 1H), 4.27 (s, 2H), 3.33-3.31 (m, 4H), 3.13 (t, J=6.6 Hz, 2H), 2.90-2.86 (m, 4H), 2.72 (t, J=6.4 Hz, 2H), 1.84-1.74 (m, 4H) ppm.

### Example 161

### (R)-5-(3-(3-((3-Chloro-4-(trifluoromethoxy)benzyl)amino)propoxy)pyrrolidin-1-yl)benzo[c][2,6]naphthyridine-8-carboxylic acid

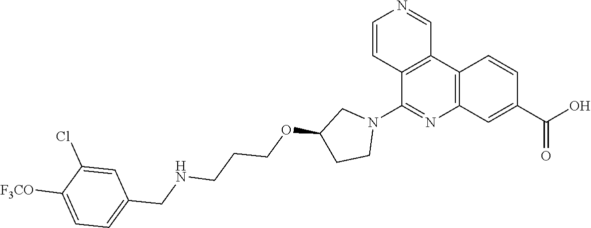 [SMILES: O=C(O)c1ccc2c(c1)nc(N1CC[C@@H](OCCCNCc3ccc(OC(F)(F)F)c(Cl)c3)C1)c1ccncc12]

To a solution of compound 1.579 (200 mg, 339.56 μmol) in MeOH (3 mL), THF (5 mL) and H₂O (5 mL) was added LiOH·H₂O (28.50 mg, 679.11 μmol). The reaction mixture was stirred at 20° C. for 1 h. The reaction mixture was acidified with aq. 1 N HCl to pH 5 and concentrated in vacuo. The residue was purified (PM180) to afford EXAMPLE 161 (133 mg, 229.27 μmol, 67.5% yield) as a yellow solid.

LCMS (AM3): rt=0.781 min, (575.2 [M+H]⁺), 96.9% purity.

¹H NMR (400 MHz, DMSO-d₆) δ: 10.08 (s, 1H), 8.81 (d, J=6.0 Hz, 1H), 8.73 (d, J=8.4 Hz, 1H), 8.21-8.18 (m, 2H), 7.85 (dd, J=8.4, 1.6 Hz, 1H), 7.58 (d, J=2.0 Hz, 1H), 7.46-7.34 (m, 2H), 4.20-4.16 (m, 1H), 4.14-4.09 (m, 1H), 4.08-4.00 (m, 1H), 3.87-3.81 (m, 1H), 3.79-3.76 (m, 1H), 3.64 (s, 2H), 3.55-3.45 (s, 2H), 2.53-2.51 (m, 2H), 2.10-2.03 (m, 2H), 1.68-1.60 (quin, 2H) ppm.

### Example 162

### (S)-5-(3-(3-((3-Chloro-4-(trifluoromethoxy)benzyl)amino)propoxy)pyrrolidin-1-yl)benzo[c][2,6]naphthyridine-8-carboxylic acid

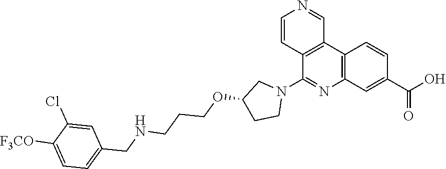 [SMILES: O=C(O)c1ccc2c(c1)nc(N1CC[C@H](OCCCNCc3ccc(OC(F)(F)F)c(Cl)c3)C1)c1ccncc12]

To a solution of compound 1.585 (260 mg, 441.42 μmol) in MeOH (3 mL), THF (5 mL) and H₂O (5 mL) was added LiOH·H₂O (37.05 mg, 882.85 μmol) at 20° C. The reaction mixture was stirred at 20° C. for 12 h. The reaction mixture was acidified with aq. 1 N HCl to pH 5 and concentrated in vacuo. The residue was purified (PM181) to afford EXAMPLE 162 (74.1 mg, 127.81 μmol, 28.9% yield) as a yellow solid.

LCMS (AM3): rt=0.781 min, (575.1 [M+H]⁺), 95.7% purity.

¹H NMR (400 MHz, DMSO-d₆) δ: 10.07 (s, 1H), 8.79 (d, J=5.6 Hz, 1H), 8.71 (d, J=8.4 Hz, 1H), 8.19-8.17 (m, 2H), 7.85 (dd, J=8.4, 1.6 Hz, 1H), 7.59 (d, J=2.0 Hz, 1H), 7.45-7.36 (m, 2H), 4.20-4.16 (m, 1H), 4.1 (dd, J=12.0, 4.8 Hz, 1H), 4.06-4.00 (m, 1H), 3.86-3.80 (m, 1H), 3.78-3.75 (m, 1H), 3.67 (s, 2H), 3.57-3.47 (m, 2H), 2.55-2.53 (m, 2H), 2.09-2.02 (m, 2H), 1.70-1.62 (quin, 2H) ppm.

### Example 163

### N-(3-((8-(2H-tetrazol-5-yl)benzo[c][2,6]naphthyridin-5-yl)amino)propyl)-3-((3-chloro-4-cyclobutoxybenzyl)amino)propanamide

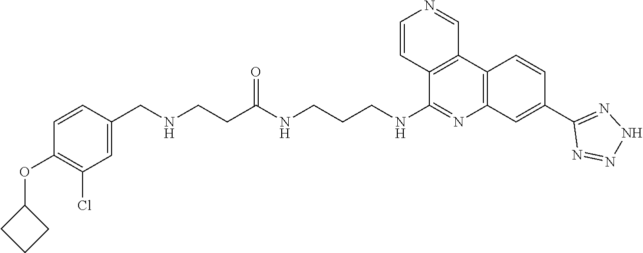 [SMILES: O=C(CCNCc1ccc(OC2CCC2)c(Cl)c1)NCCCNc1nc2cc(-c3nn[nH]n3)ccc2c2cnccc12]

A solution of compound 1.549 (80 mg, 186.96 μmol), DIPEA (48.33 mg, 373.93 μmol) and 3-chloro-4-cyclobutoxybenzaldehyde (Ref: WO2022185041, 39.39 mg, 186.96 μmol) in MeOH (10 mL) was stirred at 20° C. for 3.5 h, then NaBH₃CN (35.25 mg, 560.89 μmol) was added. The reaction mixture was stirred at 20° C. for 1 h. The reaction mixture was filtered and the filtrate was concentrated in vacuo. The residue was purified (PM182) to afford EXAMPLE 163 (14.19 mg, 24.21 μmol, 13.0% yield) as a yellow solid.

LCMS (AM3): rt=0.791 min, (586.2 [M+H]⁺), 100% purity.

¹H NMR (400 MHz, MeOH-d₄) δ: 9.90 (s, 1H), 8.71 (d, J=5.6 Hz, 1H), 8.60 (d, J=8.4 Hz, 1H), 8.29 (d, J=1.2 Hz, 1H), 8.09 (d, J=5.6 Hz, 1H), 8.03 (dd, J=8.4, 1.6 Hz, 1H), 7.36 (d, J=2.0 Hz, 1H), 7.15 (dd, J=8.4, 2.4 Hz, 1H), 6.77 (d, J=8.4 Hz, 1H), 4.68-4.54 (m, 1H), 4.00 (s, 2H), 3.81 (t, J=6.4 Hz, 2H), 3.35 (t, J=6.0 Hz, 2H), 3.20 (t, J=6.8 Hz, 2H), 2.77 (t, J=6.4 Hz, 2H), 2.46-2.38 (m, 2H), 2.14-2.04 (m, 2H), 1.97-1.92 (quin, 2H), 1.88-1.62 (m, 2H) ppm.

### Example 164

### N-(3-((8-(2H-tetrazol-5-yl)benzo[c][2,6]naphthyridin-5-yl)amino)propyl)-3-((3-chloro-4-(trifluoromethoxy)benzyl)amino)propanamide

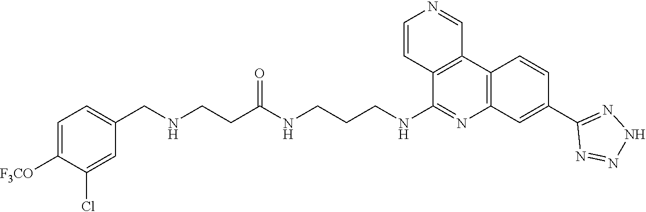

A mixture of EXAMPLE 63 (220 mg, 395.00 μmol), NH₄Cl (211.29 mg, 3.95 mmol) and sodium azide (0.970 g, 14.92 mmol) in DMF (15 mL) was stirred at 100° C. for 12 h. The reaction mixture was diluted with H₂O (30 mL) and extracted with ethyl acetate (50 mL×2). The combined organic layer was washed (brine, 80 mL×2), dried (Na₂SO₄), filtered and concentrated in vacuo. The residue was purified (PM183) to afford EXAMPLE 164 (22.86 mg, 32.02 μmol, 8.11% yield, TFA salt) as a yellow solid.

LCMS (AM3): rt=0.814 min, (600.3 [M+H]⁺), 100% purity.

¹H NMR (400 MHz, MeOH-d₄) δ: 10.06 (s, 1H), 8.94 (d, J=5.6 Hz, 1H), 8.85 (d, J=8.4 Hz, 1H), 8.64 (d, J=1.2 Hz, 1H), 8.34 (d, J=5.6 Hz, 1H), 8.12 (dd, J=8.4, 1.6 Hz, 1H), 7.77 (d, J=2.0 Hz, 1H), 7.57-7.49 (qd, 2H), 4.32 (s, 2H), 3.87 (t, J=6.4 Hz, 2H), 3.44-3.38 (m, 4H), 2.79 (t, J=6.4 Hz, 2H), 2.13-2.05 (m, 2H) ppm.

### Example 165

### N-(2-(4-(2-(((2-chloro-[1,1′-biphenyl]-4-yl)methyl)amino)ethyl)-1H-1,2,3-triazol-1-yl)ethyl)-8-(2H-tetrazol-5-yl)benzo[c][2,6]naphthyridin-5-amine

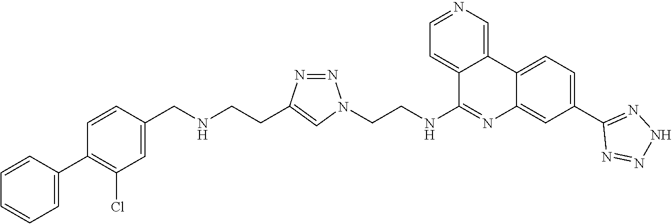

To a solution of compound 1.572 (40 mg, 57.30 μmol) in MeOH (15 mL) was added K₂CO₃ (79.19 mg, 572.99 μmol), then the reaction mixture was heated to 70° C. and stirred for 12 h. The reaction mixture was filtered and concentrated in vacuo. The residue was purified (PM136) to afford EXAMPLE 165 (22.93 mg, 32.02 μmol, 55.9% yield, TFA salt) as a yellow gum.

LCMS (AM3): rt=0.809 min, (602.5 [M+H]⁺), 100% purity.

¹H NMR (400 MHz, DMSO-d₆) δ: 10.13 (s, 1H), 8.99 (br s, 2H), 8.91 (d, J=3.2 Hz, 1H), 8.89 (s, 1H), 8.38 (d, J=1.6 Hz, 1H), 8.28 (br s, 1H), 8.19 (d, J=5.6 Hz, 1H), 8.07 (s, 1H), 8.02 (dd, J=8.4, 2.0 Hz, 1H), 7.72 (s, 1H), 7.51-7.38 (m, 7H), 4.78 (t, J=6.0 Hz, 2H), 4.24-4.20 (m, 2H), 4.10-4.05 (m, 2H), 3.24-3.16 (m, 2H), 2.99 (t, J=7.6 Hz, 2H) ppm.

### Example 166

### 5-(3-(3-((3-Chloro-4-(trifluoromethoxy)benzyl)amino)propanamido)propyl)-4-oxo-4,5-dihydro-3H-pyrazolo[3,4-c]quinoline-7-carboxamide

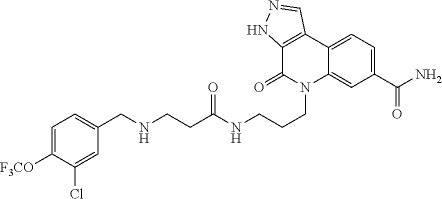 [SMILES: NC(=O)c1ccc2c3cn[nH]c3c(=O)n(CCCNC(=O)CCNCc3ccc(OC(F)(F)F)c(Cl)c3)c2c1]

To a solution of compound 1.820 (50 mg, 0.07 mmol) in MeOH (4 mL) was added a solution of HCl in MeOH (4 M, 1 mL) at RT. The mixture was stirred at RT for 1 h. The reaction mixture was concentrated in vacuo and the residue was purified (PM136) to afford EXAMPLE 166 (17.5 mg, 0.03 mmol, 43.6% yield, HCl salt) as a white solid.

LCMS (AM3): rt=0.811 min, (565.1 [M+H]⁺), 100% purity.

¹H NMR (400 MHz, MeOH-d₄) δ: 8.52 (s, 1H), 8.14 (d, J=8.0 Hz, 1H), 8.09 (s, 1H), 7.84 (dd, J=8.0, 1.2 Hz, 1H), 7.81 (d, J=2.0 Hz, 1H), 7.59 (dd, J=8.4, 2.0 Hz, 1H), 7.53-7.50 (m, 1H), 4.51 (t, J=7.6 Hz, 2H), 4.36 (s, 2H), 3.40-3.36 (t, 2H), 3.36-3.32 (t, 2H), 2.78 (t, J=6.0 Hz, 2H), 2.07-2.00 (quin, 2H) ppm.

### Example 167

### 5-((2-((3-(((2-Chloro-[1,1′-biphenyl]-4-yl)methyl)amino)cyclobutyl)methoxy)ethyl)amino)benzo[c][2,6]naphthyridine-8-carboxamide

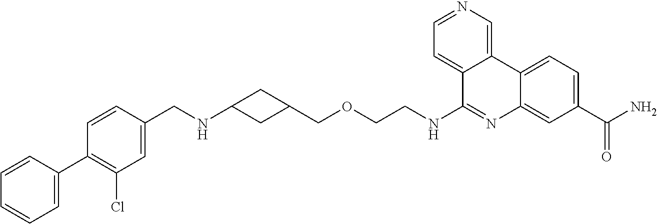

A mixture of compound 1.665 (50 mg, 65.20 μmol), HATU (37.19 mg, 97.80 μmol), DIPEA (8.43 mg, 65.20 μmol) and NH₄Cl (6.98 mg, 130.40 μmol) in DMF (1 mL) was stirred at 20° C. for 0.5 h. The reaction mixture was diluted with H₂O (30 mL) and extracted with EA (25 mL×2). The combined organic layer was washed (brine, 25 mL×2), dried (Na₂SO₄), filtered and concentrated in vacuo to give a yellow solid. The solid was dissolved in MeOH (0.5 mL) and a solution of HCl in MeOH (0.5 mL, 4 M) and stirred at 20° C. for 1.5 h. The reaction mixture was concentrated in vacuo and purified (PM184) to afford EXAMPLE 167 (7.89 mg, 13.94 μmol, 18.5% yield) as a yellow solid.

LCMS (AM3): rt=0.801 min, (566.2 [M+H]⁺), 100% purity.

¹H NMR: (400 MHz, MeOH-d₄) δ: 10.07 (br s, 1H), 8.98 (br s, 1H), 8.77 (d, J=8.4 Hz, 1H), 8.44 (s, 1H), 8.40 (d, J=5.6 Hz, 1H), 8.02 (dd, J=8.4, 1.2 Hz, 1H), 7.60 (s, 1H), 7.46-7.35 (m, 7H), 4.08-4.06 (m, 4H), 3.90 (t, J=5.6 Hz, 2H), 3.71-3.63 (m, 1H), 3.58 (d, J=6.0 Hz, 2H), 2.48-2.33 (m, 3H), 2.03-1.95 (m, 2H) ppm.

### Example 168

### 5-((2-((5-((3-Chlorophenyl)amino)pentyl)oxy)ethyl)amino)benzo[c][2,6]naphthyridine-8-carboxamide

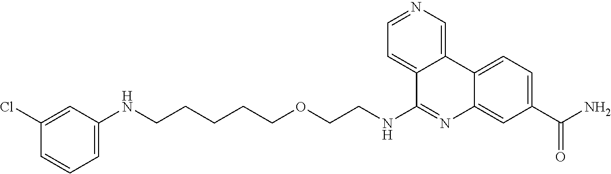

To a solution of compound 1.464 (150 mg, 286.24 μmol) in DMF (10 mL) was added HATU (130.99 mg, 344.49 μmol), DIPEA (121.43 mg, 939.52 μmol) followed by NH₄Cl (117.26 mg, 2.19 mmol) at 20° C. The resulting mixture was stirred at 20° C. for 2 h. The mixture was diluted with H₂O (50 mL) and extracted with EA (50 mL×2). The combined organic phase was washed (brine, 50 mL), dried (Na₂SO₄) and concentrated in vacuo. The residue was purified (PM136) to afford EXAMPLE 168 (33.45 mg, 40.79 μmol, 13.0% yield, TFA salt) as a yellow solid.

LCMS (AM3): rt=0.832 min, (478.2 [M+H]⁺), 100% purity.

¹H NMR (400 MHz, MeOH-d₄) δ: 10.03 (s, 1H), 8.99 (d, J=6.0 Hz, 1H), 8.74 (d, J=8.4 Hz, 1H), 8.45 (d, J=1.3 Hz, 1H), 8.40 (d, J=5.6 Hz, 1H), 8.04 (dd, J=1.3, 8.4 Hz, 1H), 7.13 (t, J=8.1 Hz, 1H), 6.80 (d, J=7.9 Hz, 1H), 6.71 (t, J=1.8 Hz, 1H), 6.64 (dd, J=1.9, 8.2 Hz, 1H), 4.10 (t, J=4.9 Hz, 2H), 3.88 (t, J=4.9 Hz, 2H), 3.57 (t, J=6.1 Hz, 2H), 2.88 (t, J=7.3 Hz, 2H), 1.63-1.56 (quin, 2H), 1.55-1.46 (quin, 2H), 1.38-1.30 (quin, 2H) ppm.

### Example 169

### 5-(3-(4-(((5-Cyclobutoxy-1H-indol-2-yl)methyl)amino)butoxy) azetidin-1-yl)benzo[c][2,6]naphthyridine-8-carboxamide

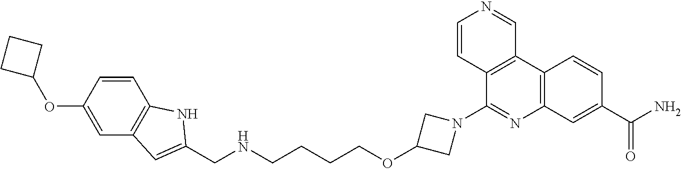 [SMILES: NC(=O)c1ccc2c(c1)nc(N1CC(OCCCCNCc3cc4cc(OC5CCC5)ccc4[nH]3)C1)c1ccncc12]

To a solution of 5-(3-(4-Aminobutoxy) azetidin-1-yl)benzo[c][2,6]naphthyridine-8-carboxamide (ref: WO2022185041, 300 mg, 744.65 μmol) and compound 1.420 (160.68 mg, 746.47 μmol) in MeOH (10 mL) was added DIPEA (310.65 mg, 2.40 mmol). The mixture was stirred at 20° C. for 12 h before NaBH(OAc)₃ (632.83 mg, 2.99 mmol) was added. The mixture was stirred at 20° C. for 1 h. The mixture was concentrated in vacuo and the residue was purified (PM185) to afford EXAMPLE 169 (46.73 mg, 81.27 μmol, 10.9% yield) as a yellow solid.

LCMS (AM3): rt=0.759 min, (565.3 [M+H]⁺), 98.3% purity.

¹H NMR (400 MHz, MeOH-d₄) δ: 9.95 (s, 1H), 8.73 (d, J=5.6 Hz, 1H), 8.63 (d, J=8.4 Hz, 1H), 8.24 (s, 1H), 7.92 (d, J=6.0 Hz, 1H), 7.87 (dd, J=8.4, 1.6 Hz, 1H), 7.15 (d, J=8.8 Hz, 1H), 6.83 (d, J=2.4 Hz, 1H), 6.61 (dd, J=8.8, 2.4 Hz, 1H), 6.25 (s, 1H), 4.71-4.67 (m, 2H), 4.56 (t, J=7.2 Hz, 1H), 4.46-4.41 (m, 1H), 4.35-4.32 (m, 2H), 3.88 (s, 2H), 3.50 (t, J=5.6 Hz, 2H), 2.68 (t, J=6.8 Hz, 2H), 2.43-2.35 (m, 2H), 2.13-2.02 (m, 2H), 1.83-1.74 (m, 2H), 1.69-1.65 (m, 4H) ppm.

### Example 170

### (S)-5-(3-(3-((3-chloro-4-(trifluoromethoxy)benzyl)amino)propoxy)pyrrolidin-1-yl)benzo[c][2,6]naphthyridine-8-carboxamide

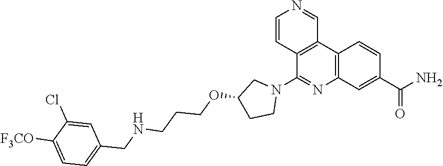 [SMILES: NC(=O)c1ccc2c(c1)nc(N1CC[C@H](OCCCNCc3ccc(OC(F)(F)F)c(Cl)c3)C1)c1ccncc12]

A mixture of compound 1.700 (100 mg, 208.57 μmol), DIPEA (53.91 mg, 417.14 μmol) and 3-chloro-4-(trifluoromethoxy)benzaldehyde (CAS 83279-39-4, 46.84 mg, 208.57 μmol) in MeOH (3 mL) was stirred at 20° C. for 12 h, then NaBH(OAc)₃ (132.61 mg, 625.72 μmol) was added. The reaction mixture was stirred at 20° C. for 3 h. The reaction mixture was filtered and concentrated in vacuo and the crude product was purified (PM186) to afford EXAMPLE 170 (13.03 mg, 20.83 μmol, 10.0% yield, FA salt) as a yellow solid.

LCMS (AM3): rt=0.755 min, (574.3 [M+H]⁺), 99.1% purity.

¹H NMR (400 MHz, MeOH-d₄) δ: 9.86 (s, 1H), 8.71 (d, J=5.6 Hz, 1H), 8.52 (d, J=8.8 Hz, 2H), 8.17-8.15 (m, 2H), 7.80 (dd, J=8.4, 1.6 Hz, 1H), 7.63 (d, J=2.0 Hz, 1H), 7.43-7.34 (m, 2H), 4.28 (br s, 1H), 4.16-4.10 (m, 2H), 4.08 (s, 2H), 3.90-3.82 (m, 2H), 3.73-3.68 (m, 1H), 3.67-3.62 (m, 1H), 3.10 (t, J=6.8 Hz, 2H), 2.28-2.22 (m, 1H), 2.20-2.09 (m, 1H), 2.03-1.93 (m, 2H) ppm.

### Example 171

### 5-(4-(4-((3,5-Difluoro-4-(trifluoromethoxy)benzyl)amino)-1H-1,2,3-triazol-1-yl)butoxy)benzo[c][2,6]naphthyridine-8-carboxylic acid

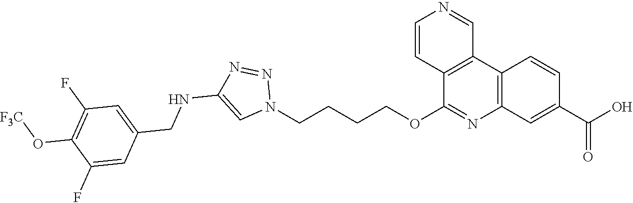 [SMILES: O=C(O)c1ccc2c(c1)nc(OCCCCn1cc(NCc3cc(F)c(OC(F)(F)F)c(F)c3)nn1)c1ccncc12]

To a solution of compound 1.882 (30 mg, 81.90 μmol) in THF (5 mL) was added NaH (6.55 mg, 163.81 μmol, 60% wt.) at 0° C. The mixture was then warmed to RT and stirred for 1 h, compound 1.1 (25 mg, 91.68 μmol) was added. The reaction mixture was then stirred at RT for 16 h. The reaction mixture was quenched by addition of MeOH (1 mL) and the resulting mixture was concentrated in vacuo. The residue was purified (PM187) to afford EXAMPLE 171 (2.85 mg, 4.81 μmol, 5.9% yield) as white solid.

LCMS (AM3): rt=0.949 min, (589.2 [M+H]⁺), 99.4% purity.

¹H NMR (400 MHz, MeOH-d₄) δ: 10.05 (s, 1H), 8.87 (d, J=4.8 Hz, 1H), 8.76 (d, J=8.4 Hz, 1H), 8.48 (s, 1H), 8.24 (d, J=5.2 Hz, 1H), 8.17 (dd, J=8.4 Hz, 1.2 Hz, 1H), 7.24 (s, 1H), 7.16 (d, J=8.8 Hz, 2H), 4.68 (t, J=6.4 Hz, 2H), 4.42 (t, J=6.8 Hz, 2H), 4.32 (s, 2H), 2.20-2.13 (m, 2H), 1.98-1.91 (m, 2H) ppm.

### Example 172

### 5-(2-(4-(2-((3,5-Difluoro-4-(trifluoromethoxy)benzyl)amino)ethyl)-1H-1,2,3-triazol-1-yl)ethoxy)benzo[c][2,6]naphthyridine-8-carboxylic acid

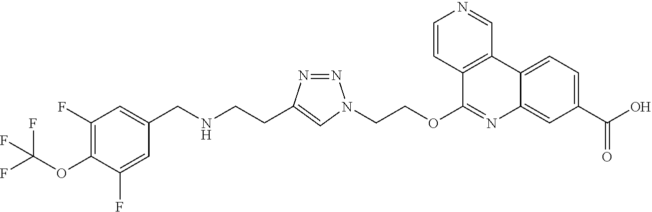

A solution of compound 1.887 (120 mg, 317.14 μmol), 3,5-Difluoro-4-(trifluoromethoxy)benzaldehyde (ref: WO2022185041, 100.39 mg, 443.99 μmol) and DIPEA (204.93 mg, 1.59 mmol) in THF (3 mL) was stirred at 50° C. for 12 h, then NaBH(OAc)₃ (201.64 mg, 951.41 μmol) was added. The reaction mixture was stirred at 50° C. for 2 h. The mixture was concentrated in vacuo to give a residue. The residue was purified (PM188) to afford EXAMPLE 172 (37.88 mg, 60.25 μmol, 19.00% yield) as white solid.

LCMS (AM3): rt=0.721 min, (589.3 [M+H]⁺), 94.9% purity.

¹H NMR (400 MHz, METHANOL-d₄) δ: 9.78 (s, 1H), 8.71 (d, J=5.6 Hz, 1H), 8.44 (d, J=8.4 Hz, 1H), 8.19 (d, J=1.2 Hz, 1H), 8.11 (s, 1H), 8.01-7.96 (m, 2H), 7.29 (d, J=8.8 Hz, 2H), 5.05-5.02 (m, 2H), 4.98-4.95 (m, 2H), 4.05 (s, 2H), 3.13 (t, J=6.4 Hz, 2H), 3.05 (t, J=6.4 Hz, 2H) ppm.

### Example 173

### 5-(2-((3-((3,5-Difluoro-4-(trifluoromethoxy)benzyl)amino)cyclobutyl)methoxy)ethoxy)benzo[c][2,6]naphthyridin e-8-carboxylic acid

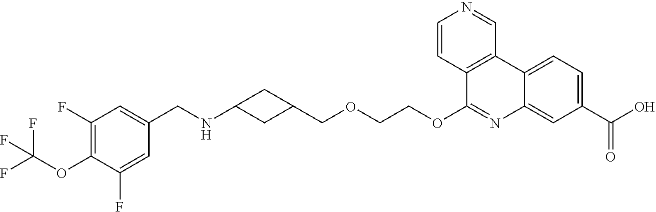

A mixture of compound 1.858 (150 mg, 362.82 μmol, FA salt), DIPEA (148.40 mg, 1.15 mmol) and 3,5-Difluoro-4-(trifluoromethoxy)benzaldehyde (ref: WO2022185041, 150 mg, 663.42 μmol) in THF (10 mL) was stirred at 50° C. for 12.5 h, then NaBH(OAc)₃ (230.69 mg, 1.09 mmol) was added. The reaction mixture was stirred at 50° C. for 3 h. The reaction mixture was concentrated in vacuo to give the residue. The residue was purified (PM189) to afford EXAMPLE 173 (46.94 mg, 81.28 μmol, 22.4% yield) as white solid.

LCMS (AM3): rt=0.833 min, (578.0 [M+H]⁺), 100% purity.

¹H NMR (400 MHz, DMSO-d₆) δ: 10.18 (s, 1H), 8.94 (d, J=5.2 Hz, 1H), 8.90 (dd, J=8.4, 2.0 Hz, 1H), 8.35 (s, 1H), 8.10-8.07 (m, 2H), 7.30 (dd, J=9.2, 2.4 Hz, 2H), 4.75 (q, J=6.4 Hz, er2H), 3.89 (q, J=6.4 Hz, 2H), 3.58 (d, J=12.4 Hz, 2H), 3.54 (d, J=7.2 Hz, 1H), 3.49 (d, J=6.0 Hz, 1H), 3.24-3.16 (m, 0.5H), 3.03-2.96 (m, 0.5H), 2.40-2.35 (m, 0.5 H), 2.18-2.16 (m, 0.5H), 2.14-2.06 (m, 1H), 1.90-1.84 (m, 1H), 1.81-1.75 (m, 1H), 1.48-1.42 (m, 1H) ppm.

### Example 174

### (S)-5-(2-((4-((3,5-difluoro-4-(trifluoromethoxy)benzyl)amino)pentyl)oxy)ethoxy)benzo[c][2,6]naphthyridine-8-carboxylic acid

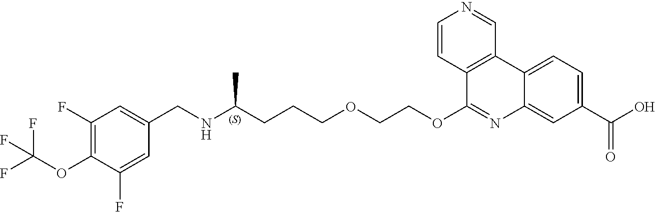

A mixture of compound 1.851 (230 mg, 622.61 μmol, FA salt), 3,5-Difluoro-4-(trifluoromethoxy)benzaldehyde (ref: WO2022185041, 230 mg, 1.02 mmol) and DIPEA (241.40 mg, 1.87 mmol) in THF (10 mL) was stirred at 20° C. for 12.5 h, then NaBH(OAc)₃ (395.87 mg, 1.87 mmol) was added. The reaction mixture was heated to 40° C. and stirred for 3 h. The reaction mixture was concentrated in vacuo. The residue was purified (PM190) to afford EXAMPLE 174 (32.73 mg, 46.94 μmol, TFA salt) as off-white solid.

LCMS (AM3): rt=0.832 min, (580.0 [M+H]⁺), 99.5% purity.

¹H NMR (400 MHz, DMSO-d₆) δ: 10.20 (s, 1H), 9.00-8.94 (m, 2H), 8.91-8.83 (m, 1H), 8.80-8.70 (m, 1H), 8.36 (s, 1H), 8.12-8.07 (m, 2H), 7.54 (d, J=8.8 Hz, 2H), 4.77 (t, J=5.2 Hz, 2H), 4.24-4.12 (m, 2H), 3.92 (t, J=4.4 Hz, 2H), 3.60-3.57 (m, 2H), 2.53-2.51 (m, 1H), 1.87-1.81 (m, 1H), 1.71-1.61 (m, 1H), 1.59-1.46 (m, 2H), 1.23 (d, J=6.8 Hz, 3H) ppm.

### Biological Assays

### Assay 1: Biochemical Assay for Inhibitors of CK2α Kinase Activity

The inhibitory activity of putative kinase inhibitors and the potency of selected compounds were determined using ADP-Glo™ assay. The kinase reaction was performed in the presence of excess peptide substrate and ATP at a concentration equivalent to Kₘ. Upon termination of the kinase reaction, remaining ATP was depleted leaving only ADP reaction product, which was converted back to ATP with a coupled luciferin/luciferase reaction. The luminescent output from the coupled reaction was quantified and correlated with the kinase activity.

CK2α (residues 2-329) was produced in Escherichia coli BL21 (DE3) for kinase activity screening. Single colonies of the cells were grown in 6×1 L of 2×TY with 100 μg/mL ampicillin at 37° C. Isopropyl thio-β-D-galactopyranoside (IPTG) was added to a final concentration of 0.4 mM to induce expression when the optical density at 600 nm reached 0.6. The cells were incubated overnight at 25° C. then harvested by centrifugation at 4,000 g for 20 minutes. The cell pellets were suspended in 20 mM Tris, 500 mM NaCl, pH 8.0 and lysed using a high pressure homogenizer. Protease inhibitor cocktail tablets (one tablet per 50 mL extract; Roche Diagnostics) and DNase I were then added. The crude cell extract was then centrifuged at 10,000 g for 45 minutes, the supernatant was filtered with a 0.22 μm filter. The soluble supernatant was applied on a Ni Sepharose Fast Flow6 column at pH 8.0, washed and eluted in 20 mM Tris pH 8.0, 500 mM NaCl, 200 mM imidazole. After overnight dialysis into 20 mM Tris, pH 8.0, 500 mM NaCl the N-terminal His6-tag was cleaved overnight by TEV protease and passed through a second metal affinity column to remove uncleaved protein and the protease. The cleaved protein was further purified on a Sepharose Q HP anion-exchange column and the main peak fraction from this column was further purified by gel filtration on a Superdex 75 16/60 HiPrep column equilibrated with Tris 20 mM, pH 8.0, 500 mM NaCl. Pure protein was concentrated to 15 mg/mL and flash frozen in liquid nitrogen.

Final assay conditions comprised 0.2 nM CK2α, 50 μM peptide substrate (RRRADDSDDDDD), 15 μM ATP in 1× reaction buffer (40 mM Tris pH7.5, 200 mM NaCl, 20 mM MgCl₂, 0.1 mg/mL BSA, 1% DMSO). The assay was conducted as follows:
1. Appropriate serial dilutions of test compound were prepared using Echo (Labcyte) and 50 nL of 100× compound in 100% DMSO transferred to the assay plate (white opaque OptiPlate-384, Perkin-Elmer).
2. Enzyme and peptide substrate were prepared in fresh reaction buffer and added to the assay plate in a total volume of 3 μl and incubated at room temperature for 15 minutes.
3. 2 μL of ATP solution freshly prepared in reaction buffer was added to start the reaction.
4. After 120 minutes, the reaction was stopped by addition of 5 μl ADP-Glo reagent (Promega V9102) and the plate incubated at room temperature for a further 60 minutes.
5. 10 μL of Kinase Detection reagent (Promega V9102) was added to assay plate and incubated for a further 30 minutes prior to reading luminescence on an Envision (Perkin-Elmer).
   Data was analysed to calculate compound IC₅₀ and Kᵢ as follows:
1. All assay plates contained 32 wells designated as 0% inhibition control wells, which were treated with vehicle only (1% DMSO) and 32 wells designated as 100% inhibition control wells, which were treated with a high concentration of non-specific kinase inhibitor in 1% DMSO.
2. Percent inhibition in each test well was calculated using the formula (MEAN_{0% inhibition control wells}−test well reading)/(MEAN_{0% inhibition control wells}−MEAN_{100% inhibition control wells})×100%.
3. IC₅₀ was determined using a standard 4-parameter fit method (Model 205, XL-fit).
4. Percent activity was calculated for each well using: (Test well reading−MEAN_{100% inhibition control wells})/(MEAN_{0% inhibition control wells}−MEAN_{100% inhibition control wells}).
5. Morrison Kᵢ was determined using Morrison Kᵢ equation (XL-fit).

### Assay 2: Biochemical Assay for Inhibitors of CLK2 Kinase Activity

The assay was conducted in the same way as described for CK2α, with final assay conditions comprising 20 nM CLK2 (Carna Biosciences-04-127), 50 μM peptide substrate (KRRRLASLR), 100 μM ATP in 1× reaction buffer (40 mM Tris pH7.5, 200 mM NaCl, 20 mM MgCl2, 0.1 mg/mL BSA, 1% DMSO).

### Assay 3: Cell-Based NanoBRET™ Assay for Inhibitor Binding to Intracellular CK2α

This assay used the NanoBRET™ System (Promega), an energy transfer technique designed to measure molecular proximity in living cells. The assay measured the apparent affinity of test compounds by competitive displacement of a NanoBRET™ tracer reversibly bound to a NanoLucR luciferase CK2α fusion protein in cells. A fixed concentration of tracer was added to cells expressing the desired NanoLucR-CK2α fusion protein to generate a BRET reporter complex. Introduction of competing compounds resulted in a dose-dependent decrease in NanoBRET™ energy transfer, which allowed quantitation of the apparent intracellular affinity of the target protein for the test compound.

The assay was conducted as follows using HCT116 cell line (ATCC CCL-247™) transiently transfected with CSNK2A2-NanoLuc® Fusion Vector (Promega NV1191):
1. Cells were resuspended to 2×10⁵ cells/mL in Opti-MEM (Invitrogen 11058021).
2. DNA complex was prepared in a final volume of 1.4 ml Opti-MEM containing 15 μg DNA and 42 μl FuGENE HD Transfection reagent (Promega E2311).
3. 20 ml cell suspension was combined with 1 ml DNA complex, added to T75 flask and incubated overnight at 37° C. in 5% CO₂ incubator.
4. Appropriate serial dilutions of test compound were prepared and 5 μl/well transferred to the assay plate (white opaque CulturPlate-384, Perkin-Elmer) using Bravo (Agilent) with 5 μl NanoBRET Tracer K-5 (Promega N2501) diluted to the recommended concentration in assay buffer (Invitrogen 11058021) and 30 μl cell suspension. The plate was incubated for 2 hours at 37° C. in 5% CO₂ incubator.
5. 20 μl 3× complete substrate plus inhibitor solution (containing NanoBRET Nano-Glo substrate and extracellular NanoLuc inhibitor diluted to manufacturer's recommendations in assay medium) was added to each well.
6. Donor emission wavelength (450 nm) and acceptor emission wavelength (610 nm or 630 nm) were measured on the Envision (Perkin-Elmer) and BRET ratio calculated for data analysis:BRET Ratio=(Acceptorₛₐₘₚₗₑ/Donorₛₐₘₚₗₑ)×1,000.
7. All assay plates contained 32 wells designated as 0% inhibition control wells, which were treated with vehicle only (1% DMSO) and 32 wells designated as 100% inhibition control wells, which were treated with a high concentration of non-specific kinase inhibitor in 1% DMSO. Percent inhibition in each test well was calculated using the formula (MEAN_{0% inhibition control wells}−test well reading)/(MEAN_{0% inhibition control wells}−MEAN_{100% inhibition control wells})×100%.
8. IC₅₀ was determined using a standard 4-parameter fit method (Model 205, XL-fit).

### Biological Data:

| | | | Assay 3: |
|---|---|---|---|
| | Assay 1: | Assay 2: | NanoBRET ™ |
| | CK2α | CLK2 | assay for binding |
| Example | Enzyme | Enzyme | to intracellular |
| No | IC₅₀ (nM) | IC₅₀ (nM) | CK2α (nM) |
| 1 | 0.21 | 23.71 | |
| 2 | 0.39 | 216.20 | 1156.00 |
| 3 | 0.18 | 164.10 | |
| 4 | 6.92 | 526.20 | 14730.00 |
| 5 | 0.21 | 651.10 | 2704.00 |
| 6 | 0.21 | 400.10 | 3934.00 |
| 7 | 0.46 | 1519.00 | 571.70 |
| 8 | 4.09 | 681.90 | 2070.00 |
| 9 | 1.10 | 877.60 | 4769.00 |
| 10 | 4.62 | 648.90 | 5963.00 |
| 11 | 6.51 | 872.20 | 6101.00 |
| 12 | 12.16 | 540.20 | 29100.00 |
| 13 | 3.29 | 431.90 | 9151.00 |
| 14 | 6.42 | 860.40 | 32460.00 |
| 15 | 6.04 | 918.40 | 12610.00 |
| 16 | 6.66 | 987.80 | 46540.00 |
| 17 | 4.90 | 414.00 | 17230.00 |
| 18 | 8.17 | 676.70 | 2740.00 |
| 19 | 0.81 | 1927.00 | 512.20 |
| 20 | 3.13 | 387.70 | 6043.00 |
| 21 | 6.15 | 347.80 | 18010.00 |
| 22 | 8.33 | 511.40 | 26300.00 |
| 23 | 2.53 | 1536.00 | 2906.00 |
| 24 | 1.18 | 1306.00 | 2809.00 |
| 25 | 1.01 | 1743.00 | 2323.00 |
| 26 | 0.91 | 1143.00 | 1128.00 |
| 27 | 0.54 | 976.60 | 770.50 |
| 28 | 0.89 | 1099.00 | 1713.00 |
| 29 | 13.96 | 960.50 | 5697.00 |
| 30 | 1.25 | 842.10 | 904.20 |
| 31 | 0.78 | 1237.00 | 3179.00 |
| 32 | 18.26 | 1898.00 | 25320.00 |
| 33 | 14.84 | 2577.00 | 15340.00 |
| 34 | 13.46 | 3354.00 | 1939.00 |
| 35 | 0.44 | 475.00 | 419.80 |
| 36 | 0.43 | 1610.00 | 1900.00 |
| 37 | 4.07 | 2400.00 | 3267.00 |
| 38 | 0.24 | 175.00 | 338.00 |
| 39 | 2.00 | 584.30 | 3088.00 |
| 40 | 1.36 | 1640.00 | 4907.00 |
| 41 | 9.21 | 5869.00 | 1929.00 |
| 42 | 7.48 | 14200.00 | 5053.00 |
| 43 | 4.16 | 1026.00 | 1654.00 |
| 44 | 2.01 | 439.00 | 176.10 |
| 45 | 13.25 | 2360.00 | 1487.00 |
| 46 | 0.20 | 298.50 | 24.79 |
| 47 | 1.52 | 500.00 | 225.20 |
| 48 | 7.58 | 1744.00 | 1460.00 |
| 49 | 0.43 | 258.40 | 68.14 |
| 50 | 8.92 | 354.80 | 1206.00 |
| 51 | 0.37 | 3012.00 | 96.68 |
| 52 | 15.22 | 931.40 | 2053.00 |
| 53 | 2.02 | 213.90 | 1645.00 |
| 54 | 1.09 | 1258.00 | 148.70 |
| 55 | 10.78 | 947.00 | 2536.00 |
| 56 | 1.25 | *36230 | 433.40 |
| 57 | 0.77 | 2684.00 | 364.00 |
| 58 | 2.10 | 1345.00 | 784.50 |
| 59 | 1.62 | 9142.00 | 3261.00 |
| 60 | 0.75 | 1787.00 | 357.20 |
| 61 | 2.49 | 6889.00 | 213.50 |
| 62 | 0.71 | 1985.00 | 370.10 |
| 63 | 1.05 | 2382.00 | 205.60 |
| 64 | 0.19 | 870.10 | 841.60 |
| 65 | 15.63 | 1807.00 | 4666.00 |
| 66 | 0.40 | 223.50 | 4511.00 |
| 67 | 0.32 | 382.50 | 5794.00 |
| 68 | 0.72 | 911.20 | 2397.00 |
| 69 | 0.26 | 235.30 | 3808.00 |
| 70 | 7.79 | 2785.00 | 39020.00 |
| 71 | 18.53 | 1229.00 | 4715.00 |
| 72 | 1.57 | 199.10 | 1087.00 |
| 73 | 7.44 | 3431.00 | 2067.00 |
| 74 | 0.30 | *17270 | 5846.00 |
| 75 | 12.00 | 866.70 | 28160.00 |
| 76 | 1.10 | 1732.00 | 1908.00 |
| 77 | 5.17 | 849.40 | 3639.00 |
| 78 | 0.87 | 836.20 | 5393.00 |
| 79 | 0.54 | 467.60 | 32830.00 |
| 80 | 0.47 | 317.20 | 78.49 |
| 81 | 0.92 | 160.90 | 862.90 |
| 82 | 1.38 | 1582.00 | 311.10 |
| 83 | 6.38 | 4587.00 | 740.20 |
| 84 | 6.18 | 401.70 | 1196.00 |
| 85 | 0.76 | 3430.00 | 300.90 |
| 86 | 1.08 | 312.00 | 276.30 |
| 87 | 1.42 | 636.00 | 691.70 |
| 88 | 0.50 | 1412.00 | 140.00 |
| 89 | 9.33 | 1212.00 | 8514.00 |
| 90 | 1.21 | 941.80 | 3710.00 |
| 91 | 14.74 | 1574.00 | 7798.00 |
| 92 | 12.40 | 24690.00 | 2512.00 |
| 93 | 15.18 | 11640.00 | 966.20 |
| 94 | 13.43 | 2274.00 | >50000 |
| 95 | 0.53 | 2282.00 | 382.70 |
| 96 | 5.98 | 760.30 | 933.60 |
| 97 | 2.49 | 739.20 | 165.80 |
| 98 | 0.32 | 12060.00 | 90.44 |
| 99 | 0.43 | 173.80 | 165.90 |
| 100 | 0.30 | 370.60 | 45.23 |
| 101 | 0.36 | 367.60 | 69.72 |
| 102 | 0.33 | 228.70 | 40.15 |
| 103 | 0.25 | 393.80 | 36.48 |
| 104 | 0.18 | 265.80 | 27.50 |
| 105 | 0.49 | 215.40 | 368.10 |
| 106 | 0.24 | 609.60 | 86.02 |
| 107 | 0.83 | 284.80 | 248.60 |
| 108 | 0.31 | 375.70 | 83.69 |
| 109 | 0.70 | 650.70 | 118.70 |
| 110 | 0.51 | 224.40 | 162.40 |
| 111 | 0.75 | 825.10 | 526.80 |
| 112 | 1.81 | 1181.00 | 2059.00 |
| 113 | 17.86 | 2643.00 | 4298.00 |
| 114 | 7.92 | 1940.00 | 21730.00 |
| 115 | 3.40 | 915.40 | 11430.00 |
| 116 | 15.05 | 2353.00 | 9691.00 |
| 117 | 0.46 | 696.60 | 44.53 |
| 118 | 2.76 | 601.00 | 17690.00 |
| 119 | 4.37 | 423.50 | 17800.00 |
| 120 | 1.99 | 709.10 | 8608.00 |
| 121 | 1.55 | 661.90 | 3958.00 |
| 122 | 5.09 | 860.80 | 3661.00 |
| 123 | 24.11 | 537.60 | 18130.00 |
| 124 | 0.56 | 1394.00 | 746.40 |
| 125 | 0.84 | 418.20 | 1673.00 |
| 126 | 1.01 | 473.60 | 1369.00 |
| 127 | 0.33 | 752.90 | 222.30 |
| 128 | 0.37 | 683.70 | 202.40 |
| 129 | 4.43 | 234.70 | 1079.00 |
| 130 | 0.87 | 761.10 | 543.40 |
| 131 | 5.54 | 449.60 | 4142.00 |
| 132 | 13.18 | 441.90 | 5333.00 |
| 133 | 0.30 | 1425.00 | 68.93 |
| 134 | 0.55 | 4273.00 | 87.13 |
| 135 | 12.98 | 2389.00 | 2270.00 |
| 136 | 1.45 | 7989.00 | 318.90 |
| 137 | 6.49 | 6200.00 | 1914.00 |
| 138 | 29.81 | 2668.00 | 6303.00 |
| 139 | 0.90 | 2327.00 | 1325.00 |
| 140 | 17.52 | 3187.00 | 10250.00 |
| 141 | 6.88 | 5255.00 | 3588.00 |
| 142 | 2.59 | 1343.00 | 3399.00 |
| 143 | 1.42 | 4160.00 | 539.70 |
| 144 | 1.89 | 2726.00 | 408.00 |
| 145 | 4.18 | 8950.00 | 829.30 |
| 146 | 0.90 | 3182.00 | 2576.00 |
| 147 | 3.58 | 6444.00 | 1531.00 |
| 148 | 1.43 | 5387.00 | 1206.00 |
| 149 | 0.41 | 360.90 | 24610.00 |
| 150 | 0.37 | 404.10 | 857.80 |
| 151 | 3.54 | 744.60 | 333.90 |
| 152 | 0.22 | 195.60 | 595.20 |
| 153 | 0.45 | 149.90 | 179.50 |
| 154 | 0.20 | 231.80 | 3765.00 |
| 155 | 0.31 | 119.00 | 51.39 |
| 156 | 0.36 | 407.90 | 6621.00 |
| 157 | 0.43 | 412.40 | 22620.00 |
| 158 | 4.53 | 760.70 | 19210.00 |
| 159 | 2.30 | 50.27 | 561.40 |
| 160 | 0.82 | 480.40 | 85.63 |
| 161 | 2.27 | 51.87 | 558.80 |
| 162 | 0.36 | 93.47 | 67.23 |
| 163 | 0.32 | >50000 | 14130.00 |
| 164 | 0.42 | 23570.00 | 8893.00 |
| 165 | 0.47 | >50000 | 9288.00 |
| 166 | 2.10 | 737.50 | 3140.00 |
| 167 | 2.84 | 1237.00 | 1304.00 |
| 168 | 10.57 | 3496.00 | 898.00 |
| 169 | 9.29 | 1198.00 | 5883.00 |
| 170 | 13.83 | 1051.00 | 1789.00 |
| 171 | 0.29 | 557.60 | 26.83 |
| 172 | 2.04 | 2366.00 | 325.40 |
| 173 | 0.37 | 342.60 | 26.10 |
| 174 | 0.41 | 441.50 | 7.58 |

### REFERENCES

Battistutta & Lolli (2011). Structural and functional determinants of protein kinase CK2α: facts and open questions. Mol. Cell. Biochem., 2011, 356, 67-73.
Niefind et al (2001). Crystal structure of human protein kinase CK2: insights into basic properties of the CK2 holoenzyme. EMBO J. 2001, 20, 5320-5331.
Meggio & Pinna (2003). One-thousand-and-one substrates of protein kinas CK2. The FASEB Journal 17, 349-368.
Behan et al (2019). Prioritizaion of cancer therapeutic targets using CRISPR-Cas9 screens. Nature 568, 511-516.
Lin et al (2011). Overexpression of Nuclear Protein Kinase CK2α Catalytic Subunit (CK2α) as a Poor Prognosticator in Human Colorectal Cancer. PLOS ONE 6, 17193-
Ortega et al (2014). Mining CK2 in cancer. PLOS ONE 9, 115609-
Di Maira et al. (2019). The protein kinase CK2 contributes to the malignant phenotype of cholangiocarcinoma cells. Oncogenesis 8, 61-
Zhan et al (2017). Wnt signaling in cancer. Oncogene 36, 1461-1473.
Gao & Wang (2006). Casein Kinase 2 is activated and essential for Wnt/β-Catenin signaling. Journal of Biological Chemistry 281, 189394-18400.
Dowling et al (2016). Potent and selective CK2 Kinase Inhibitors with effects on Wnt pathway signaling in vivo. ACS Med. Chem. Lett. 7, 300-305.
Zakharia et al. (2019). Pre-clinical in vitro and in vivo evidence of an antitumour effect of CX-4945, a casein kinase II inhibitor, in cholangiocarcinoma. Translational Oncology 12, 143-153.Brear et al. (2016). Specific inhibition of CK2α from an anchor outside the active site. Chem. Sci. 7, 6839-6845.
Ruzzene & Pinna (2010). Addiction to protein kinase CK2: A common denominator of diverse cancer cells? Biochimica et Biophysica Acta 1804, 499-504.
Montenarh (2016). Protein Kinase CK2 in DNA Damage and repair. Transl. Cancer Res. 5, 49-63.
Gordon, D. E., Jang, G. M., Bouhaddou, M. et al. (2020). A SARS-COV-2 protein interaction map reveals targets for drug repurposing. Nature 583, 459-468.

1. A compound of formula I, or a pharmaceutically acceptable salt thereof: 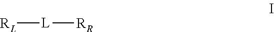
wherein:
L is linker that separates R_{L} from R_{R} by 7 to 13, 8 to 12, 9 to 11, or 8, 9 or 10 bond lengths, with the proviso that L is not a group of the formula: 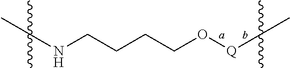
wherein Q is selected from formula Ia or Ib shown below: 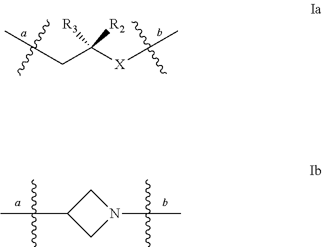
wherein:
bond a in formulae Ia and Ib corresponds with bond a in the above formula and bond b in formulae Ia and Ib corresponds with bond b in the above formula;
R₂ and R₃ are each independently selected from hydrogen or methyl; and
X is NH or O;
R_{L} is selected from one of formulae Ic, Id, Ie, If, Ig, Ih, Ii, Ij, Ik or Il shown below: 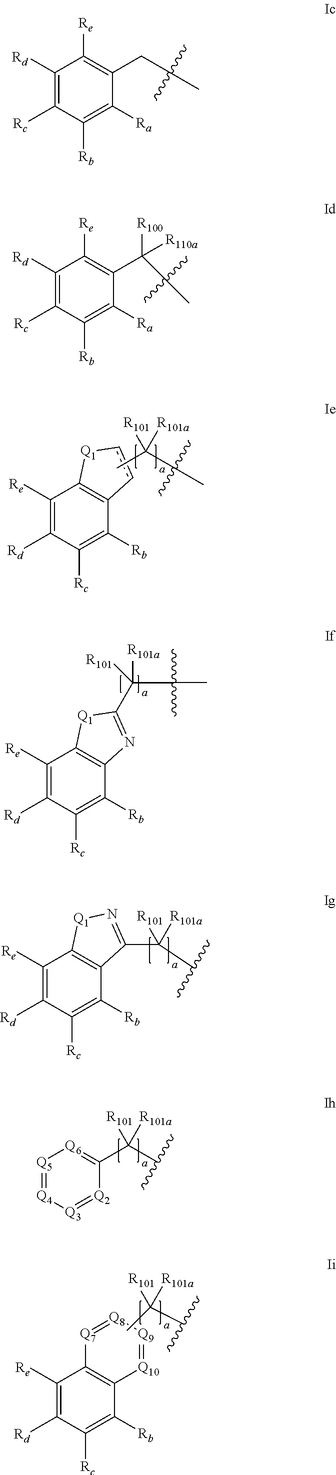 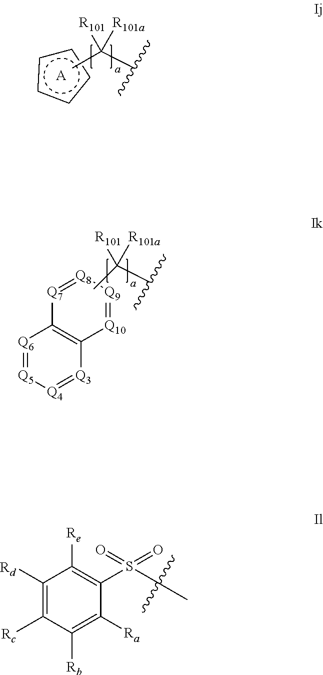
wherein:
Rₐ and Rₑ are each independently selected from hydrogen, methyl or halo;
R_{b} and R_{d} are each independently selected from hydrogen, halo, cyano, (1-4C)alkyl, (3-6C)cycloalkyl,
—[CH₂]₀₋₃-(1-4C)alkoxy,
—[CH₂]₀₋₃—C(O)NH₂,
—[CH₂]₀₋₃—C(O)NH(1-4C)alkyl,
—[CH₂]₀₋₃—C(O)N[(1-4C)alkyl]₂,
—[CH₂]₀₋₃—NH₂,
—[CH₂]₀₋₃—NH(1-4C)alkyl,
—[CH₂]₀₋₃—N[(1-4C)alkyl]₂,
—[CH₂]₀₋₃—S(O)_{q}-(1-4C)alkyl (wherein q is 0, 1 or 2),
—[CH₂]₀₋₃—C(O)(1-4C)alkyl,
—[CH₂]₀₋₃—C(O)OH,
—[CH₂]₀₋₃—C(O)O-(1-4C)alkyl,
—[CH₂]₀₋₃—N(R_{f})C(O)-(1-4C)alkyl (wherein R_{f} is hydrogen or methyl),
—[CH₂]₀₋₃—S(O)₂NH(1-4C)alkyl,
—[CH₂]₀₋₃—S(O)₂N[(1-4C)alkyl]₂,
[CH₂]₀₋₃—N(R_{g})SO₂-(1-4C)alkyl (wherein R_{g} is hydrogen or methyl),
a group of the formula: 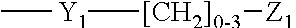
wherein Y₁ is absent, —O—, —NH—, —NMe-, —S—, —S(O)— or —S(O)₂—; and
Z₁ is (3-6C)cycloalkyl, phenyl, a 4- to 6-membered heterocyclyl or 5 or 6-membered heteroaryl;
and wherein:
any alkyl, alkoxy, cycloalkyl or —[CH₂]— moiety within a R_{b} and R_{d} substituent group is optionally substituted by one or more substituents selected from halo, hydroxy, cyano, amino, —C(O)OH, —C(O)NH₂, (1-2C)alkoxy, or (3-4C)cycloalkoxy; and
Z₁ is optionally substituted by one or more substituents selected from: halo, hydroxy, cyano, amino, —C(O)OH, —C(O)NH₂, (1-2C)alkoxy, (1-2C)alkyl, (3-4C)cycloalkyl, (3-4C)cycloalkoxy, —C(O)NH(1-2C)alkyl, —C(O)N[(1-2C)alkyl]₂, —NH(1-2C)alkyl, —N[(1-2C)alkyl]₂, —S(O)_{q}-(1-2C)alkyl (wherein q is 0, 1 or 2), —C(O)(1-2C)alkyl, —C(O)O-(1-2C)alkyl, —N(R_{f})C(O)-(1-2C)alkyl, —S(O)₂NH(1-2C)alkyl, —S(O)₂N[(1-2C)alkyl]₂, or —NHSO₂-(1-2C)alkyl, and wherein any (1-2C)alkoxy, (1-2C)alkyl, (3-4C)cycloalkyl or (3-4C)cycloalkoxy group is optionally substituted by one or more substituents selected from halo, cyano, hydroxy, (1-2C)alkyl, (1-2C)alkoxy or (1-2C)alkoxy-(1-2C)alkyl;
R_{c} is selected from hydrogen, halo, cyano, —C(O)NH₂, (1-4C)alkyl,
—[CH₂]₀₋₃-(1-4C)alkoxy,
—[CH₂]₀₋₃-(3-6C)cycloalkoxy,
—[CH₂]₀₋₃—C(O)NH₂,
—[CH₂]₀₋₃—C(O)NH(1-4C)alkyl,
—[CH₂]₀₋₃—C(O)N[(1-4C)alkyl]₂,
—[CH₂]₀₋₃—NH₂,
—[CH₂]₀₋₃—NH(1-4C)alkyl,
—[CH₂]₀₋₃—N[(1-4C)alkyl]₂,
—[CH₂]₀₋₃—S(O)_{q}-(1-4C)alkyl (wherein q is 0, 1 or 2),
—[CH₂]₀₋₃—C(O)(1-4C)alkyl,
—[CH₂]₀₋₃—C(O)OH,
—[CH₂]₀₋₃—C(O)O-(1-4C)alkyl,
—[CH₂]₀₋₃—N(Rₕ) C(O)-(1-4C)alkyl (wherein Rₕ is hydrogen or methyl),
—[CH₂]₀₋₃—S(O)₂NH(1-4C)alkyl,
—[CH₂]₀₋₃—S(O)₂N[(1-4C)alkyl]₂,
—[CH₂]₀₋₃—N(Rᵢ)SO₂-(1-4C)alkyl (wherein Rᵢ is hydrogen or methyl),
a group of the formula: 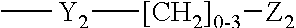
wherein Y₂ is absent, —O—, —NH—, —NMe-, —S—, —S(O)— or —S(O)₂—; and
Z₂ is (3-6C)cycloalkyl, phenyl, a 4- to 6-membered heterocyclyl or 5 or 6-membered heteroaryl;
and wherein:
any alkyl, alkoxy, cycloalkyl or —[CH₂]— moiety within a R_{c} substituent group is optionally substituted by one or more substituents selected from halo, hydroxy, cyano, amino, —C(O)OH, —C(O)NH₂, (1-2C)alkoxy, or (3-4C)cycloalkoxy; and
Z₂ is optionally substituted by one or more substituents selected from: halo, hydroxy, cyano, amino, —C(O)OH, —C(O)NH₂, (1-2C)alkoxy, (1-2C)alkyl, (3-4C)cycloalkyl, (3-4C)cycloalkoxy, —C(O)NH(1-2C)alkyl, —C(O)N[(1-2C)alkyl]₂, —NH(1-2C)alkyl, —N[(1-2C)alkyl]₂, —S(O)_{q}-(1-2C)alkyl (wherein q is 0, 1 or 2), —C(O)(1-2C)alkyl, —C(O)O-(1-2C)alkyl, —N(R_{f})C(O)-(1-2C)alkyl, —S(O)₂NH(1-2C)alkyl, —S(O)₂N[(1-2C)alkyl]₂, or —NHSO₂-(1-2C)alkyl, and wherein any (1-2C)alkoxy, (1-2C)alkyl, (3-4C)cycloalkyl or (3-4C)cycloalkoxy group is optionally substituted by one or more substituents selected from halo, cyano, hydroxy, (1-2C)alkyl, (1-2C)alkoxy or (1-2C)alkoxy-(1-2C)alkyl;
R₁₀₀ is (1-2C)alkyl optionally substituted by hydroxy or halo;
R₁₀₀ₐ is selected from hydrogen or methyl;
or R₁₀₀ and R₁₀₀ₐ are linked to form a cyclopropyl or cyclobutyl ring;
R₁₀₁ is hydrogen or (1-2C)alkyl optionally substituted by hydroxy or halo;
R₁₀₁ₐ is selected from hydrogen or methyl;
or R₁₀₁ and R₁₀₁ₐ are linked to form a cyclopropyl or cyclobutyl ring;
integer a is 0, 1 or 2;
Q₁ is selected from —NR₁₀₂—, —O—, —S— or —CH—;
R₁₀₂ is hydrogen or (1-2C)alkyl; [symbol]
represents a single or double bond;
Q₂ is N or CRₐ;
Q₃ is N or CR_{b};
Q₄ is N or CR_{c};
Q₅ is N or CR_{d};
Q₆ is N or CRₑ;
Rₐ, R_{b}, R_{c}, R_{d} and Rₑ are each as defined above;
with the proviso that one to three of Q₂, Q₃, Q₄, Q₅ or Q₆ is/are N;
Q₇ is N or CR_{f};
Q₈ is N or CR_{f};
Q₉ is N or CR_{f};
Q₁₀ is N or CR_{f};
with the proviso that one or two of Q₇, Q₈, Q₉ or Q₁₀ is/are N;
each R_{f} present is independently selected from hydrogen, methyl or halo;
Ring A is a five-membered heteroaryl ring optionally substituted by one R_{b} and/or one or two R_{c} substituents;
R_{R} is selected from one of formulae Im, In, Io, Ip, Iq or Ir shown below: 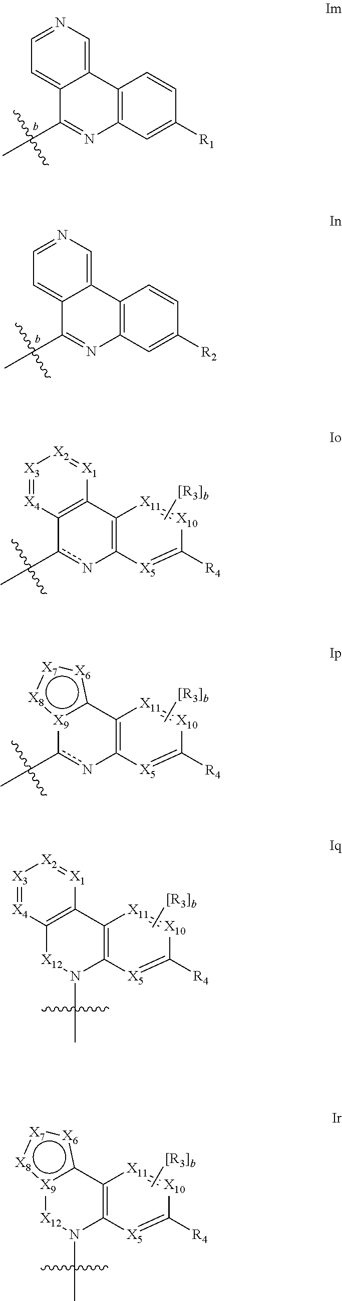
wherein: [symbol]
denotes the point of attachment; [symbol]
denotes an optional double bond;
R₁ is selected from —C(O)OH or —C(O)NH₂;
R₂ is selected from:
(i) —C(O)NHR₂ₐ or —C(O)OR₂ₐ, wherein R₂ₐ is (1-4C)alkyl optionally substituted by halo, hydroxy, amino or carboxyl;
(ii) —N(R_{2b})C(O)R_{2c}, wherein R_{2b} is hydrogen or methyl and R_{2c} is (1-4C)alkyl optionally substituted by halo, hydroxy, amino or carboxyl;
(iii) —CN;
(iv) a 5- or 6-membered heteroaryl comprising one, two, three or four nitrogen atoms;
(v) oxetanyl, tetrahydrofuranyl or tetrahydropyranyl;
R₃ is selected from methyl, fluoro or chloro and integer b is 0 or 1;
R₄ is a group R₁ or R₂ as defined above;
X₁ is CH, CRₓ₁ or N;
X₂ is CH, CRₓ₂ or N;
X₃ is CH, CRₓ₃ or N;
X₄ is CH, CRₓ₄ or N;
X₅/X₁₀/X₁₁ are each independently CH or N;
with the proviso that:
if X₂ is N and R₄ is a R₁ group, then either at least one of X₁, X₃ or X₄ is N or CRₓ₁, CRₓ₃, CRₓ₄ respectively, and/or X₅/X₁₀/X₁₁ is N, and/or integer b is 1;
Rₓ₁, Rₓ₂, Rₓ₃ and Rₓ₄ are each independently selected from methyl, hydroxy, fluoro, chloro or NR₅₀R₅₁, wherein R₅₀ and R₅₁ are each independently selected from hydrogen, (1-2C)alkyl or (3-4C)cycloalkyl;
X₆ is CH, CRₓ₅, O, S, NH, NMe or N;
X₇ is CH, CRₓ₆, O, S, NH, NMe or N;
X₈ is CH, CRₓ₇, O, S, NH, NMe or N;
X₉ is C or N;
X₁₂ is CH or C(O);
with the proviso that only one of X₅/X₁₀/X₁₁ can be N; and
only one of X₆, X₇ or X₈ can be O, S, NH or NMe;
and Rₓ₅, Rₓ₆ and Rₓ₇ are each independently selected from methyl, hydroxy, fluoro, chloro or NR₅₂R₅₃, wherein R₅₂ and R₅₃ are each independently selected from hydrogen, (1-2C)alkyl or (3-4C)cycloalkyl.

2. A compound according to claim 1, or a pharmaceutically acceptable salt thereof, wherein L is linker selected from:
(i) a group of the formula 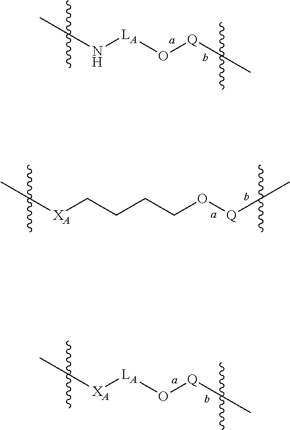
wherein L_{A} is selected from:
(i) a (4C)alkylene substituted by one or more R_{LA} substituent groups;
(ii) a (3C)alkylene or a (5C)alkylene optionally substituted by one or more R_{LA} substituent groups;
(iii) a (0-3C)alkylene-(3-6C)cycloalkylene-(0-3C)alkylene-group optionally substituted by one or more R_{LA} substituent groups;
each R_{LA} group present is selected from hydroxy, halo, (1-2C)alkyl, (1-2C)hydroxyalkyl or (1-2C)haloalkyl;
X_{A} is selected from: —O—, —S—, —SO—, —SO₂—, —N(Me)-, —C(O)—, —C(O)O—, —OC(O)—, —C(O)N(Rₓₐ)—, —N(Rₓₐ)C(O)—, —N(R_{xb})C(O)N(Rₓₐ)—, —S(O)₂N(Rₓₐ)— or —N(Rₓₐ)SO₂—, wherein Rₓₐ and R_{xb} are selected from hydrogen or methyl;
Q is selected from formula Ia or Ib shown below: 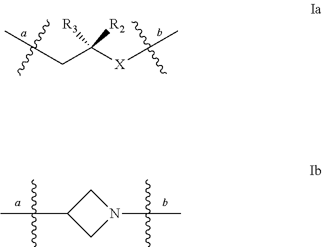
wherein:
bond a in formulae Ia and Ib corresponds with bond a in the above formula and bond b in formulae Ia and Ib corresponds with bond b in the above formula;
R₂ and R₃ are each independently selected from hydrogen or methyl; and
X is NH or O;
(ii) a group of the formula: 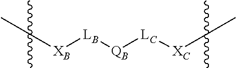
wherein:
X_{B} is selected from: —O—, —S—, —SO—, —SO₂—, —N(Rₓₐ)—, —C(O)—, —C(O)O—, —OC(O)—, —C(O)N(Rₓₐ)—, —N(Rₓₐ)C(O)—, —N(R_{xb})C(O)N(Rₓₐ)—, —S(O)₂N(Rₓₐ)— or —N(Rₓₐ)SO₂—, wherein Rₓₐ and R_{xb} are selected from hydrogen or methyl;
L_{B} is absent or selected from:
(i) a (1-5C)alkylene optionally substituted by one or more R_{LA} substituent groups;
(ii) a (0-3C)alkylene-(3-6C)cycloalkylene-(0-3C)alkylene-group optionally substituted by one or more R_{LA} substituent groups;
Q_{B} is selected from a 5 or 6-membered heteroaryl ring, a 9 or 10-membered bicyclic heteroaryl ring, a phenyl, or a 4 to 6 membered heterocyclic ring;
L_{C} is absent or selected from:
(i) a (1-5C)alkylene optionally substituted by one or more R_{LA} substituent groups;
(ii) a (0-3C)alkylene-(3-6C)cycloalkylene-(0-3C)alkylene-group optionally substituted by one or more R_{LA} substituent groups; and
X_{C} is selected from: —O—, —S—, —SO—, —SO₂—, —N(Rₓₐ)—, —C(O)—, —C(O)O—, —OC(O)—, —C(O)N(Rₓₐ)—, —N(Rₓₐ)C(O)—, —N(R_{xb})C(O)N(Rₓₐ)—, —S(O)₂N(Rₓₐ)— or —N(Rₓₐ)SO₂—, wherein Rₓₐ and R_{xb} are selected from hydrogen or methyl;
(iii) a group of the formula: 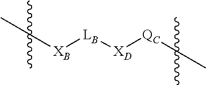
wherein:
X_{B} and L_{B} are each as defined above;
X_{D} is absent or selected from: —O—, —S—, —SO—, —SO₂, —N(Rₓₐ)—, —C(O)—, —C(O)O—, —OC(O)—, —C(O)N(Rₓₐ)—, —N(Rₓₐ)C(O)—, —N(R_{xb})C(O)N(Rₓₐ)—, —S(O)₂N(Rₓₐ)— or —N(Rₓₐ)SO₂—, wherein Rₓₐ and R_{xb} are selected from hydrogen or methyl;
Q_{C} is selected a 5 to 7 membered heterocyclic ring;
(iv) a group of the formula: 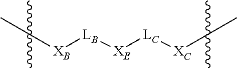
wherein:
X_{B}, L_{B}, L_{C} and X_{C} are each as defined above;
X_{E} is selected from: —O—, —S—, —SO—, —SO₂—, —N(Rₓₐ)—, —C(O)—, —C(O)O—, —OC(O)—, —C(O)N(Rₓₐ)—, —N(Rₓₐ)C(O)—, —N(R_{xb})C(O)N(Rₓₐ)—, —S(O)₂N(Rₓₐ)— or —N(Rₓₐ) SO₂—, wherein Rₓₐ and R_{xb} are selected from hydrogen or methyl;
wherein X_{E} is not —O— when X_{B} is —NH—, L_{B} is butylene, L_{C} is ethylene, and X_{C} is —NH— or —O—;
(v) a group of the formula: 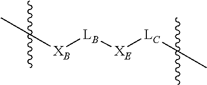
wherein:
X_{B}, L_{B}, L_{C} and X_{E} are each as defined above;
(vi) a group of the formula: 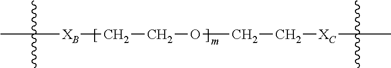
wherein:
X_{B} and X_{C} are each as defined above;
integer m is 1 or 2;

3. A compound according to claim 1 or claim 2, or a pharmaceutically acceptable salt thereof, wherein L is linker selected from:
(i) a group of the formula 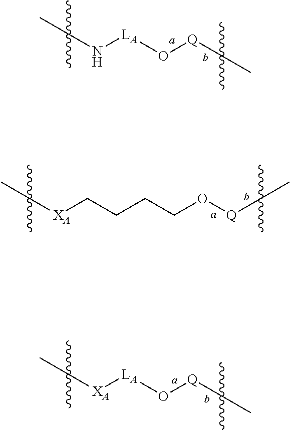
wherein L_{A} is selected from:
(i) a (4C)alkylene substituted by one or more R_{LA} substituent groups;
(ii) a (3C)alkylene or a (5C)alkylene optionally substituted by one or more R_{LA} substituent groups;
(iii) a (0-1C)alkylene-(3-6C)cycloalkylene-(1-3C)alkylene-group optionally substituted by one or more R_{LA} substituent groups;
each R_{LA} group present is selected from hydroxy, halo or (1-2C)alkyl;
X_{A} is selected from: —O—, —S—, —SO—, —SO₂—, —N(Me)-, —S(O)₂N(Rₓₐ)— or —N(Rₓₐ)SO₂—, wherein Rₓₐ and R_{xb} are selected from hydrogen or methyl;
Q is selected from formula Ia or Ib shown below: 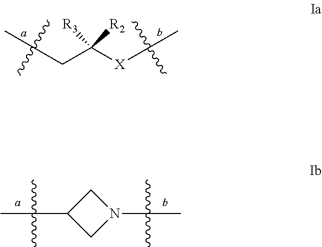
wherein:
bond a in formulae Ia and Ib corresponds with bond a in the above formula and bond b in formulae Ia and Ib corresponds with bond b in the above formula;
R₂ and R₃ are both hydrogen; and
X is NH or O;
(ii) a group of the formula: 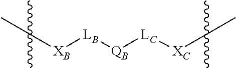
wherein:
X_{B} is selected from: —O—, —S—, —SO—, —SO₂—, —N(Rₓₐ)—, —S(O)₂N(Rₓₐ)— or —N(Rₓₐ)SO₂—, wherein Rₓₐ and R_{xb} are selected from hydrogen or methyl;
L_{B} is absent or a (1-3C)alkylene optionally substituted by one or more R_{LA} substituent groups;
Q_{B} is selected from a 5 or 6-membered heteroaryl ring, a 9 or 10-membered bicyclic heteroaryl ring, a phenyl, or a 4 to 6 membered heterocyclic ring;
L_{C} is absent or a (1-5C)alkylene optionally substituted by one or more R_{LA} substituent groups;
X_{C} is selected from: —O—, —N(Rₓₐ)—, —C(O)—, —C(O)O—, —OC(O)—, —C(O)N(Rₓₐ)—, —N(Rₓₐ)C(O)— or —N(R_{xb})C(O)N(Rₓₐ)—, wherein Rₓₐ and R_{xb} are selected from hydrogen or methyl;
(iii) a group of the formula: 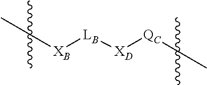
wherein:
X_{B} and L_{B} are each as defined above;
X_{D} is absent or selected from: —O—, —N(Rₓₐ)—, —C(O)—, —C(O)O—, —OC(O)— or —C(O)N(Rₓₐ)— wherein Rₓₐ and R_{xb} are selected from hydrogen or methyl;
Q_{C} is selected a 5 to 7 membered heterocyclic ring;
(iv) a group of the formula: 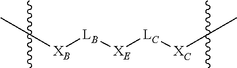
wherein:
X_{B}, L_{B}, L_{C} and X_{C} are each as defined above;
X_{E} is selected from: —O—, —N(Rₓₐ)—, —C(O)—, —C(O)O—, —OC(O)—, —C(O)N(Rₓₐ)—, —N(Rₓₐ)C(O)—, —N(R_{xb})C(O)N(Rₓₐ)—, —S(O)₂N(Rₓₐ)— or —N(Rₓₐ)SO₂—, wherein Rₓₐ and R_{xb} are selected from hydrogen or methyl;
wherein X_{E} is not —O— when X_{B} is —NH—, L_{B} is butylene, L_{C} is ethylene, and X_{C} is —NH— or —O—;
(v) a group of the formula: 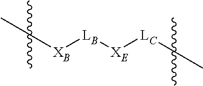
wherein:
X_{B}, L_{B}, L_{C} and X_{E} are each as defined above;
(vi) a group of the formula: 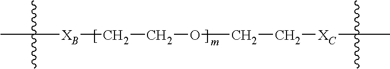
wherein:
X_{B} and X_{C} are each as defined above;
integer m is 2.

4. A compound according to any one of claims 1 to 3, or a pharmaceutically acceptable salt thereof, wherein L is linker selected from:
(i) a group of the formula 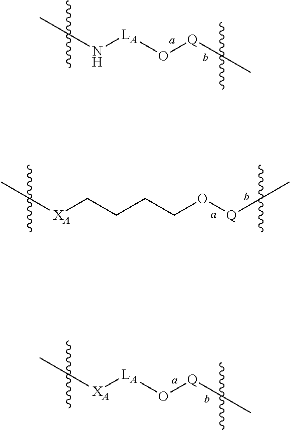
wherein L_{A} is selected from:
(i) a (4C)alkylene substituted by one R_{LA} substituent groups;
(ii) a propylene or a pentylene;
(iii) a (4C)cycloalkylene-(1-2C)alkylene-group optionally substituted by one or more R_{LA} substituent groups;
each R_{LA} group present is methyl;
X_{A} is selected from: —N(Me)-, —S(O)₂N(H)— or —N(H)SO₂—;
Q is selected from formula Ia or Ib shown below: 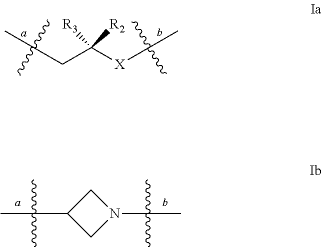
wherein:
bond a in formulae Ia and Ib corresponds with bond a in the above formula and bond b in formulae Ia and Ib corresponds with bond b in the above formula;
R₂ and R₃ are both hydrogen; and
X is NH or O;
(ii) a group of the formula: 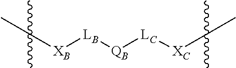
wherein:
X_{B} is selected from: —N(H)— or —S(O)₂N(H)—;
L_{B} is absent, methylene, ethylene, propylene or butylene;
Q_{B} is selected from a triazole, pyrrolidine, benzoimidazole, piperidine, phenyl, imidazole, pyrazole, oxazole or oxadiazole;
L_{C} is absent or selected from ethylene, propylene, —C(CH₃)₂CH₂CH₂— or butylene;
X_{C} is selected from: —O—, —N(H)— or —N(Me)-;
(iii) a group of the formula: 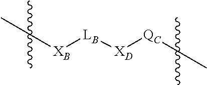
wherein:
X_{B} and L_{B} are each as defined above;
X_{D} is absent or selected from: —O—, —N(H)—;
Q_{C} is a pyrrolidine, piperazine or piperidine;
(iv) a group of the formula: 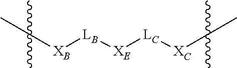
wherein:
X_{B}, L_{B}, L_{C} and X_{C} are each as defined above;
X_{E} is selected from: —O—, —N(H)—, —N(Me)-, —C(O)N(H)—, —C(O)N(Me)- —N(H)C(O)—, N(Me)C(O)— —N(H)C(O)N(H)—, N(Me)C(O)N(H)—, N(H)C(O)N(Me)-, N(Me)C(O)N(H)—, —S(O)₂N(H)—, —S(O)₂N(Me)-, —N(H)SO₂—or —N(Me)SO₂—;
wherein X_{E} is not —O— when X_{B} is —NH—, L_{B} is butylene, L_{C} is ethylene, and X_{C} is —NH— or —O—;
(v) a group of the formula: 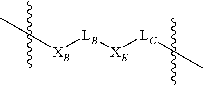
wherein:
X_{B}, L_{B}, L_{C} and X_{E} are each as defined above;
(vi) a group of the formula: 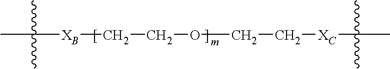
wherein:
X_{B} and X_{C} are each as defined above;
integer m is 2.

5. A compound according to any one of the preceding claims, or a pharmaceutically acceptable salt thereof, wherein L is linker selected from:

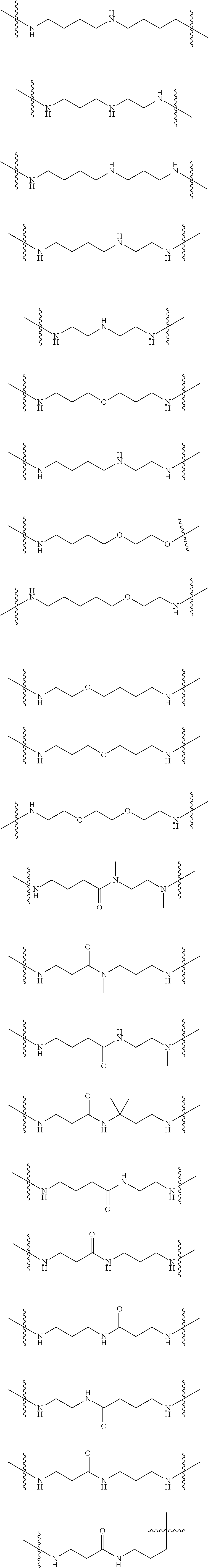

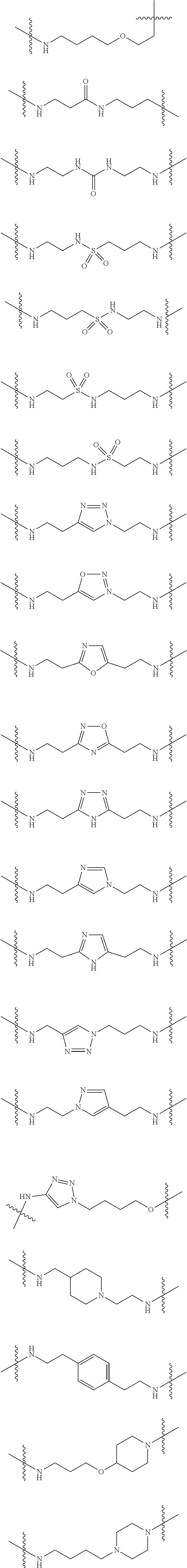

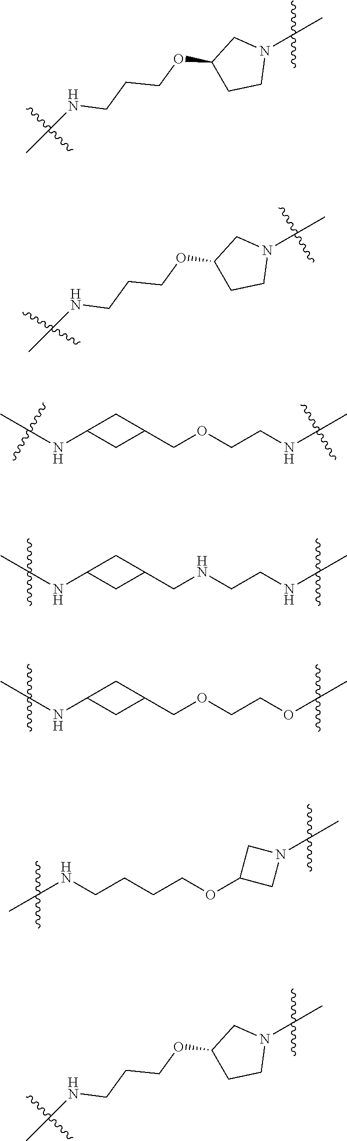

6. A compound according to any one of the preceding claims, or a pharmaceutically acceptable salt thereof, wherein R_{L} is selected from one of formulae Ic, Id, Ie, If, Ig, Ih, Ii or Ij shown below: 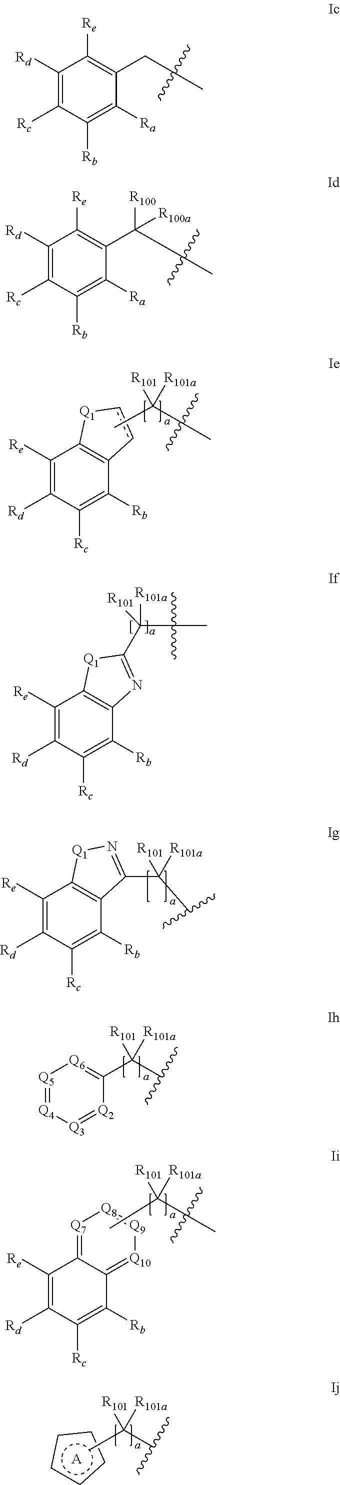
wherein: [symbol]
denotes the point of attachment; and [symbol]
denotes an optional double bond.

7. A compound according to any one of the preceding claims, or a pharmaceutically acceptable salt thereof, wherein R_{L} is selected from one of the formulae shown below: 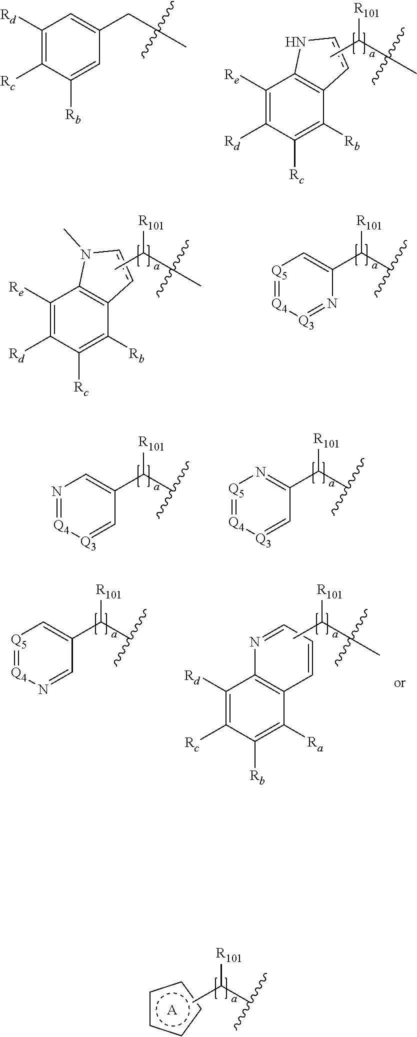
wherein: [symbol]
denotes the point of attachment; and [symbol]
denotes an optional double bond.

8. A compound according to any one of the preceding claims, or a pharmaceutically acceptable salt thereof, wherein R_{L} is selected from one of the formulae shown below: 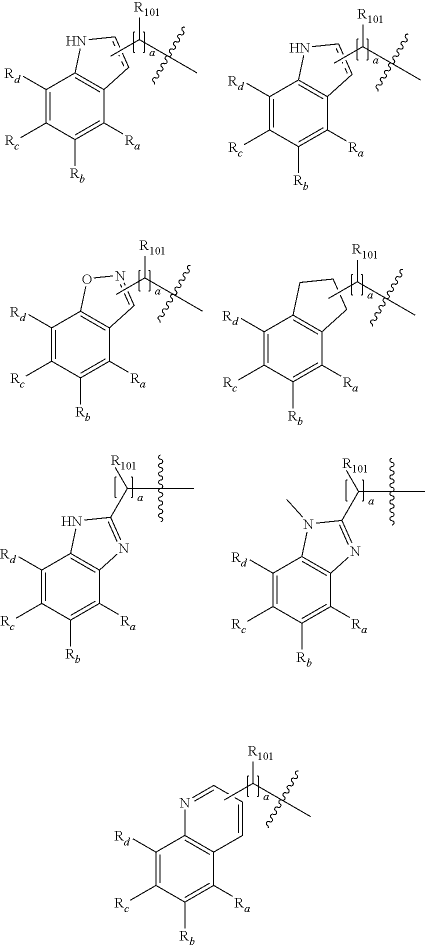
wherein: [symbol]
denotes the point of attachment; and [symbol]
denotes an optional double bond.

9. A compound according to any one of the preceding claims, or a pharmaceutically acceptable salt thereof, wherein Rₐ and Rₑ are each independently selected from hydrogen, methyl, fluoro, chloro or bromo.

10. A compound according to any one of the preceding claims, or a pharmaceutically acceptable salt thereof, wherein Rₐ and Rₑ are both hydrogen.

11. A compound according to any one of the preceding claims, or a pharmaceutically acceptable salt thereof, wherein R_{b} and R_{d} are each independently selected from hydrogen, halo, cyano, (1-4C)alkyl, (3-6C)cycloalkyl
—[CH₂]₀₋₂-(1-2C)alkoxy,
—[CH₂]₀₋₂—C(O)NH₂,
—[CH₂]₀₋₂—C(O)NH(1-2C)alkyl,
—[CH₂]₀₋₂—C(O)N[(1-2C)alkyl]₂,
—[CH₂]₀₋₃—NH₂,
—[CH₂]₀₋₂—NH(1-2C)alkyl,
—[CH₂]₀₋₂—N[(1-2C)alkyl]₂,
—[CH₂]₀₋₂—S(O)_{q}-(1-2C)alkyl (wherein q is 0, 1 or 2),
—[CH₂]₀₋₂—C(O)(1-2C)alkyl, —[CH₂]₀₋₂—C(O)OH,
—[CH₂]₀₋₂—C(O)O-(1-2C)alkyl,
—[CH₂]₀₋₂—NHC(O)-(1-2C)alkyl,
—[CH₂]₀₋₂—S(O)₂NH(1-2C)alkyl,
—[CH₂]₀₋₂—S(O)₂N[(1-2C)alkyl]₂,
—[CH₂]₀₋₂—NHSO₂-(1-2C)alkyl,
a group of the formula: 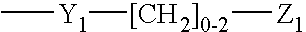
wherein Y₁ is absent, —O—, —NH—, —NMe-, —S—, —S(O)— or —S(O)₂—; and
Z₁ is (3-6C)cycloalkyl, phenyl, a 4- to 6-membered heterocyclyl or 5 or 6-membered heteroaryl;
and wherein:
any alkyl, alkoxy, cycloalkyl or —[CH₂]— moiety within a R_{b} and R_{d} substituent group is optionally substituted by one or more substituents selected from halo, hydroxy, cyano, amino, —C(O)OH, —C(O)NH₂, (1-2C)alkoxy, or (3-4C)cycloalkoxy; and
Z₁ is optionally substituted by one or more substituents selected from: halo, hydroxy, cyano, amino, —C(O)OH, —C(O)NH₂, (1-2C)alkoxy, (1-2C)alkyl, (3-4C)cycloalkyl, (3-4C)cycloalkoxy, —C(O)NH(1-2C)alkyl, —C(O)N[(1-2C)alkyl]2, —NH(1-2C)alkyl, —N[(1-2C)alkyl]2, —S(O)_{q}-(1-2C)alkyl (wherein q is 0, 1 or 2), —C(O)(1-2C)alkyl, —C(O)O-(1-2C)alkyl, —N(R_{f})C(O)-(1-2C)alkyl, —S(O)₂NH(1-2C)alkyl, —S(O)₂N[(1-2C)alkyl]2, or —NHSO₂-(1-2C)alkyl, and wherein any (1-2C)alkoxy, (1-2C)alkyl, (3-4C)cycloalkyl or (3-4C)cycloalkoxy group is optionally substituted by one or more substituents selected from halo, cyano, hydroxy, (1-2C)alkyl, (1-2C)alkoxy or (1-2C)alkoxy-(1-2C)alkyl;

12. A compound according to any one of the preceding claims, or a pharmaceutically acceptable salt thereof, wherein R_{b} and R_{d} are each independently selected from hydrogen, halo, cyano, (1-4C)alkyl, (3-6C)cycloalkyl,
—[CH₂]₀₋₁-(1-4C)alkoxy,
—[CH₂]₀₋₁—C(O)NH₂,
—[CH₂]₀₋₁—C(O)NH(1-4C)alkyl,
—[CH₂]₀₋₁—C(O)N[(1-4C)alkyl]₂,
—[CH₂]₀₋₁—NH(1-4C)alkyl,
—[CH₂]₀₋₁—N[(1-4C)alkyl]₂,
—[CH₂]₀₋₁—S(O)_{q}-(1-4C)alkyl (wherein q is 0, 1 or 2),
—[CH₂]₀₋₁—C(O)(1-4C)alkyl,
—[CH₂]₀₋₁—C(O)OH,
—[CH₂]₀₋₁—C(O)O-(1-4C)alkyl,
a group of the formula: 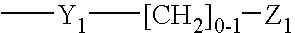
wherein Y₁ is absent, —O—, —NH—, —NMe-, —S—, —S(O)— or —S(O)₂—; and
Z₁ is (3-6C)cycloalkyl or 5 or 6-membered heteroaryl;
and wherein:
any alkyl, alkoxy, cycloalkyl or —[CH₂]— moiety within a R_{b} and R_{d} substituent group is optionally substituted by one or more substituents selected from halo, hydroxy, cyano, amino, —C(O)NH₂ or (1-2C)alkoxy; and
Z₁ is optionally substituted by one or more substituents selected from: halo, hydroxy, cyano, amino, (1-2C)alkoxy, (1-2C)alkyl or (1-2C)haloalkyl;

13. A compound according to any one of the preceding claims, or a pharmaceutically acceptable salt thereof, wherein R_{b} and R_{d} are each independently selected from hydrogen, fluoro, chloro, bromo, cyano, methyl, ethyl, methoxy, ethoxy, —CH₂OH, —CH₂OCH₃, —CH₂NH₂, —CH₂CN, —CH₂CH₂OH, —CF₃, —OCF₃, —O—CH₂CH₂OH, —O—CH₂CF₃, —C(O)NH₂, —CH₂—C(O)NH₂, —CH(CH₃) CN, —C(CH₃)₂CN, cyclopropyl, 1-cyanocyclopropyl, cyclopropylmethyl, furanylmethyl (e.g. furan-3-ylmethyl), imidazolylmethyl (e.g. imidazo-1-ylmethyl), pyrazolylmethyl (e.g. pyrazol-4-ylmethyl), oxazolylmethyl (e.g. oxazo-4-ylmethyl).

14. A compound according to any one of the preceding claims, or a pharmaceutically acceptable salt thereof, wherein R_{c} is selected from hydrogen, halo, cyano, —C(O)NH₂, (1-3C)alkyl,
—[CH₂]₀₋₂-(1-3C)alkoxy,
—[CH₂]₀₋₂-(3-6C)cycloalkoxy,
—[CH₂]₀₋₂—NH₂,
—[CH₂]₀₋₂—C(O)NH₂,
—[CH₂]₀₋₂—C(O)NH(1-3C)alkyl,
—[CH₂]₀₋₂—C(O)N[(1-3C)alkyl]₂,
—[CH₂]₀₋₂—NH(1-3C)alkyl,
—[CH₂]₀₋₂—N[(1-3C)alkyl]₂,
—[CH₂]₀₋₂—S(O)_{q}-(1-3C)alkyl (wherein q is 0, 1 or 2),
—[CH₂]₀₋₂—C(O)(1-3C)alkyl,
—[CH₂]₀₋₂—C(O)OH,
—[CH₂]₀₋₂—C(O)O-(1-3C)alkyl,
—[CH₂]₀₋₂—NHC(O)-(1-3C)alkyl (wherein Rₕ is hydrogen or methyl),
—[CH₂]₀₋₂—S(O)₂NH(1-3C)alkyl,
—[CH₂]₀₋₂—S(O)₂N[(1-3C)alkyl]₂,
—[CH₂]₀₋₂—NHSO₂-(1-3C)alkyl (wherein Rᵢ is hydrogen or methyl),
a group of the formula: 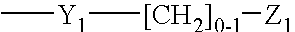
wherein Y₂ is absent, —O—, —NH—, —NMe-, —S—, —S(O)— or —S(O)₂—; and
Z₂ is (3-6C)cycloalkyl, phenyl, a 4- to 6-membered heterocyclyl or 5 or 6-membered heteroaryl;
and wherein:
any alkyl, alkoxy, cycloalkyl or —[CH₂]— moiety within a R_{c} substituent group is optionally substituted by one or more substituents selected from halo, hydroxy, cyano, amino, —C(O)OH, —C(O)NH₂, (1-2C)alkoxy, or (3-4C)cycloalkoxy; and
Z₂ is optionally substituted by one or more substituents selected from: halo, hydroxy, cyano, amino, —C(O)OH, —C(O)NH₂, (1-2C)alkoxy, (1-2C)alkyl, (3-4C)cycloalkyl, (3-4C)cycloalkoxy, —C(O)NH(1-2C)alkyl, —C(O)N[(1-2C)alkyl]₂, —NH(1-2C)alkyl, —N[(1-2C)alkyl]₂, —S(O)_{q}-(1-2C)alkyl (wherein q is 0, 1 or 2), —C(O)(1-2C)alkyl, —C(O)O-(1-2C)alkyl, —N(R_{f}) C(O)-(1-2C)alkyl, —S(O)₂NH(1-2C)alkyl, —S(O)₂N[(1-2C)alkyl]₂, or —NHSO₂-(1-2C)alkyl, and wherein any (1-2C)alkoxy, (1-2C)alkyl, (3-4C)cycloalkyl or (3-4C)cycloalkoxy group is optionally substituted by one or more substituents selected from halo, cyano, hydroxy, (1-2C)alkyl, (1-2C)alkoxy or (1-2C)alkoxy-(1-2C)alkyl.

15. A compound according to any one of the preceding claims, or a pharmaceutically acceptable salt thereof, wherein R_{c} is selected from hydrogen, halo, cyano, —C(O)NH₂, (1-3C)alkyl, —[CH₂]₀₋₁-(1-2C)alkoxy, or a group of the formula: 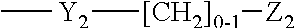
wherein Y₂ is absent, —O—, —NH— or —NMe- and
Z₂ is (3-6C)cycloalkyl, phenyl, or 5 or 6-membered heteroaryl;
and wherein:
any alkyl, alkoxy, cycloalkyl or —[CH₂]— moiety within a R_{c} substituent group is optionally substituted by one or more substituents selected from halo, hydroxy, cyano or amino; and
Z₂ is optionally substituted by one or more substituents selected from: halo, hydroxy, cyano, amino, —C(O)OH, —C(O)NH₂, (1-2C)alkoxy, (1-2C)alkyl, (3-4C)cycloalkyl, —C(O)(1-2C)alkyl, or —C(O)O-(1-2C)alkyl, and wherein any (1-2C)alkoxy, (1-2C)alkyl, (3-4C)cycloalkyl or (3-4C)cycloalkoxy group is optionally substituted by one or more substituents selected from halo, cyano, hydroxy, (1-2C)alkyl, (1-2C)alkoxy or (1-2C)alkoxy-(1-2C)alkyl.

16. A compound according to any one of the preceding claims, or a pharmaceutically acceptable salt thereof, wherein R_{c} is selected from hydrogen, chloro, —CF₃, —OCF₃, cyclopropyl, —O-cyclopropyl, —O-cyclobutyl, phenyl or 6-membered heteroaryl.

17. A compound according to any one of the preceding claims, or a pharmaceutically acceptable salt thereof, wherein
R₁₀₀ is methyl optionally substituted by hydroxy or halo;
R₁₀₀ₐ is selected from hydrogen or methyl;
R₁₀₁ is hydrogen or methyl optionally substituted by hydroxy or halo;
R₁₀₁ₐ is hydrogen;
integer a is 0 or 1;
Q₁ is selected from —NH—, —NMe- —O—, or —CH—; [symbol]
represents a double bond;
Q₂ is N or CRₐ;
Q₃ is N or CR_{b};
Q₄ is N or CR_{c};
Q₅ is N or CR_{d};
Q₆ is N or CRₑ;
Q₇ is N or CR_{f};
Q₈ is N or CR_{f};
Q₉ is N or CR_{f};
Q₁₀ is N or CR_{f};
with the proviso that one or two of Q₇, Q₈, Q₉ or Q₁₀ is/are N;
each R_{f} present is independently selected from hydrogen, methyl, fluoro, chloro or bromo
Ring A is a five-membered heteroaryl ring comprising one, two or three nitrogen atoms optionally substituted by one R_{b} and/or one R_{c} substituent.

18. A compound according to any one of the preceding claims, or a pharmaceutically acceptable salt thereof, wherein R_{R} is selected from any one of the formulae shown below: 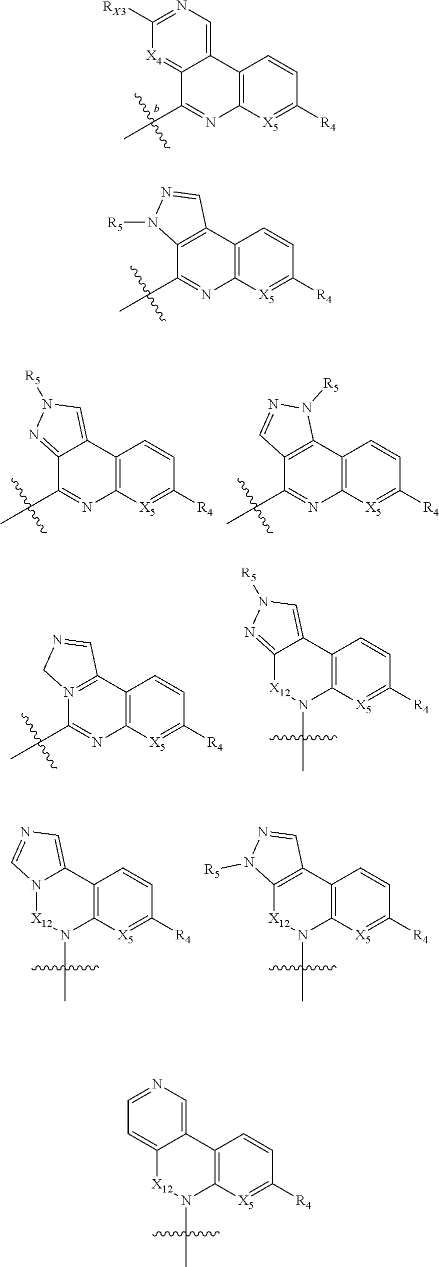
wherein: [symbol]
denotes the point of attachment and
R₅ is hydrogen or methyl.

19. A compound according to any one of the preceding claims, or a pharmaceutically acceptable salt thereof, wherein R₃ is selected from methyl, fluoro or chloro and integer b is 0.

20. A compound according to any one of the preceding claims, or a pharmaceutically acceptable salt thereof, wherein R₂ is selected from:
(i) —C(O)NHR₂ₐ or —C(O)OR₂ₐ, wherein R₂ₐ is (1-2C)alkyl optionally substituted by hydroxy, amino or carboxyl;
(ii) —N(H)C(O)R_{2c}, wherein R_{2c} is (1-2C)alkyl optionally substituted by halo, hydroxy, amino or carboxyl;
(iii) —CN;
(iv) a 5- or 6-membered heteroaryl comprising two, three or four nitrogen atoms;
(v) oxetanyl, tetrahydrofuranyl or tetrahydropyranyl.

21. A compound according to any one of the preceding claims, or a pharmaceutically acceptable salt thereof, wherein
X₁ is CH;
X₂ is N;
X₃ is CH or CRₓ₃;
X₄ is CH or N;
X₅ is CH or N;
X₁₀ and X₁₁ are both CH;
with the proviso that:
(i) one, two or three of X₂, X₄ or X₅ is/are N; and
(ii) if X₂ is N and R₄ is a R₁ group, then either at least one of X₃ is CRₓ₃, X₄ is N X₅ is N, and/or integer b is 1;
Rₓ₃ is —NHMe or —NH-cyclopropyl.

22. A compound according to any one of the preceding claims, or a pharmaceutically acceptable salt thereof, wherein
X₅ is CH;
X₆ is CH, O, S, NH, NMe or N;
X₇ is CH, O, S, NH, NMe or N;
X₈ is CH, O, S, NH, NMe or N;
X₉ is C or N;
X₁₀ and X₁₁ are both CH;
with the proviso that only one of X₆, X₇ or X₈ can be O, S, NH or NMe.

23. A compound according to any one of the preceding claims, or a salt, hydrate or solvate thereof, wherein the compound is selected from any one of the following:
5-[4-({4-[({2-chloro-[1,1′-biphenyl]-4-yl}methyl)amino]butyl}amino)butyl]benzo[c]2,6-naphthyridine-8-carboxylic acid;
5-{[2-({4-[({2-chloro-[1,1′-biphenyl]-4-yl}methyl)amino]butyl}amino)ethyl]amino}benzo[c]2,6-naphthyridine-8-carboxylic acid;
5-{[3-({4-[({2-chloro-[1,1′-biphenyl]-4-yl}methyl)amino]butyl}amino)propyl]amino}benzo[c]2,6-naphthyridine-8-carboxylic acid;
5-((2-((2-(((2-chloro-[1,1′-biphenyl]-4-yl)methyl)amino)ethyl)amino)ethyl)amino)benzo[c][2,6]naphthyridine-8-carboxylic acid;
5-(3-(3-(((2-Chloro-[1,1′-biphenyl]-4-yl)methyl)amino)propanamido)propyl)-4-oxo-4,5-dihydro-3H-pyrazolo[3,4-c]quinoline-7-carboxylic acid;
5-((2-(4-(((2-Chloro-[1,1′-biphenyl]-4-yl)methyl)amino)butanamido)ethyl)amino)benzo[c][2,6]naphthyridine-8-carboxylic acid;
5-((2-((4-(((2-chloro-[1,1′-biphenyl]-4-yl)methyl)amino)butyl)amino)ethyl)amino)benzo[c][2,6]naphthyridine-8-carboxamide;
5-((2-((4-((3-Chlorobenzyl)amino)butyl)amino)ethyl)amino)benzo[c][2,6]naphthyridine-8-carboxamide;
5-((2-((4-(((2-Chloro-2′-(hydroxymethyl)-[1,1′-biphenyl]-4-yl)methyl)amino)butyl)amino)ethyl)amino)benzo[c][2,6]naphthyridine-8-carboxamide;
5-((2-((4-((3-Chloro-4-(pyridin-3-yl)benzyl)amino)butyl)amino)ethyl)amino)benzo[c][2,6]naphthyridine-8-carboxamide;
5-((2-((4-((3-Chloro-4-(pyridin-4-yl)benzyl)amino)butyl)amino)ethyl)amino)benzo[c][2,6]naphthyridine-8-carboxamide;
5-((2-((4-(((1H-Indol-2-yl)methyl)amino)butyl)amino)ethyl)amino)benzo[c][2,6]naphthyridine-8-carboxamide;
5-((2-((4-(((5-Chloro-1H-indol-2-yl)methyl)amino)butyl)amino)ethyl)amino)benzo[c][2,6]naphthyridine-8-carboxamide;
5-((2-((4-(((4-Methyl-3-phenyl-1H-pyrazol-5-yl)methyl)amino)butyl)amino)ethyl)amino)benzo[c][2,6]naphthyridine-8-carboxamide;
5-((2-((4-(((4-chloro-3-phenyl-1H-pyrazol-5-yl)methyl)amino)butyl)amino)ethyl)amino)benzo[c][2,6]naphthyridine-8-carboxamide;
5-((2-((4-(((3-Phenyl-1H-pyrazol-5-yl)methyl)amino)butyl)amino)ethyl)amino)benzo[c][2,6]naphthyridine-8-carboxamide;
5-((2-((4-(((5-Fluoro-1H-indol-2-yl)methyl)amino)butyl)amino)ethyl)amino)benzo[c][2,6]naphthyridine-8-carboxamide;
5-((2-((4-((3-Chloro-4-fluorobenzyl)amino)butyl)amino)ethyl)amino)benzo[c][2,6]naphthyridine-8-carboxamide;
5-((2-((4-((3-Chloro-4-(trifluoromethoxy)benzyl)amino)butyl)amino)ethyl)amino)benzo[c][2,6]naphthyridine-8-carboxamide;
5-((2-((4-(((6-Chloro-1H-indol-2-yl)methyl)amino)butyl)amino)ethyl)amino)benzo[c][2,6]naphthyridine-8-carboxamide;
5-((2-((4-(((6-Fluoro-1H-indol-2-yl)methyl)amino)butyl)amino)ethyl)amino)benzo[c][2,6]naphthyridine-8-carboxamide;
5-((2-((4-(((5-Methoxy-1H-indol-2-yl)methyl)amino)butyl)amino)ethyl)amino)benzo[c][2,6]naphthyridine-8-carboxamide;
5-((2-((4-(((4-methyl-5-phenylpyridin-2-yl)methyl)amino)butyl)amino)ethyl)amino)benzo[c][2,6]naphthyridine-8-carboxamide;
5-((2-((4-(((5-(Trifluoromethyl)-1H-indol-2-yl)methyl)amino)butyl)amino)ethyl)amino)benzo[c][2,6]naphthyridine-8-carboxamide;
5-((2-((4-(((2-Methoxy-[1,1′-biphenyl]-4-yl)methyl)amino)butyl)amino)ethyl)amino)benzo[c][2,6]naphthyridine-8-carboxamide;
5-((2-((4-(((2-Cyano-[1,1′-biphenyl]-4-yl)methyl)amino)butyl)amino)ethyl)amino)benzo[c][2,6]naphthyridine-8-carboxamide;
5-((2-((4-(((2-Fluoro-[1,1′-biphenyl]-4-yl)methyl)amino)butyl)amino)ethyl)amino)benzo[c][2,6]naphthyridine-8-carboxamide;
5-((2-((4-(((2-Chloro-2′-(cyanomethyl)-[1,1′-biphenyl]-4-yl)methyl)amino)butyl)amino)ethyl)amino)benzo[c][2,6]naphthyridine-8-carboxamide;
5-((2-((4-((3-Chloro-4-(pyrazin-2-yl)benzyl)amino)butyl)amino)ethyl)amino)benzo[c][2,6]naphthyridine-8-carboxamide;
5-((2-((4-(((5-Chloro-6-phenylpyridin-3-yl)methyl)amino)butyl)amino)ethyl)amino)benzo[c][2,6]naphthyridine-8-carboxamide;
5-((2-((4-(((2-Chloro-2′-(2-hydroxyethyl)-[1,1′-biphenyl]-4-yl)methyl)amino)butyl)amino)ethyl)amino)benzo[c][2,6]naphthyridine-8-carboxamide;
5-((2-((4-(((6-Chloroquinolin-2-yl)methyl)amino)butyl)amino)ethyl)amino)benzo[c][2,6]naphthyridine-8-carboxamide;
5-((2-((4-(((6-(Trifluoromethyl) quinolin-2-yl)methyl)amino)butyl)amino)ethyl)amino)benzo[c][2,6]naphthyridine-8-carboxamide;
5-((2-((4-(((5-Phenylthiazol-2-yl)methyl)amino)butyl)amino)ethyl)amino)benzo[c][2,6]naphthyridine-8-carboxamide;
5-((2-((4-((3-Chloro-4-cyclobutoxybenzyl)amino)butyl)amino)ethyl)amino)benzo[c][2,6]naphthyridine-8-carboxamide;
5-((2-((4-((3-Cyano-4-cyclobutoxybenzyl)amino)butyl)amino)ethyl)amino)benzo[c][2,6]naphthyridine-8-carboxamide;
5-((2-((4-((3-Cyano-4-(trifluoromethoxy)benzyl)amino)butyl)amino)ethyl)amino)benzo[c][2,6]naphthyridine-8-carboxamide;
5-((3-(3-(((2-Chloro-[1,1′-biphenyl]-4-yl)methyl)amino)propanamido)propyl)amino)benzo[c][2,6]naphthyridine-8-carboxylic acid;
5-((2-((3-(((2-Chloro-[1,1′-biphenyl]-4-yl)methyl)amino)propyl)amino)ethyl)amino)benzo[c][2,6]naphthyridine-8-carboxylic acid;
6-(3-(3-(((2-Chloro-[1,1′-biphenyl]-4-yl)methyl)amino)propanamido)propyl)-5,6-dihydroimidazo[1,5-c]quinazoline-8-carboxylic acid;
5-((2-((4-(((2-chloro-[1,1′-biphenyl]-4-yl)methyl)amino)butyl)amino)ethyl)amino)benzo[c][2,6]naphthyridine-8-carbonitrile;
N-(5-((2-((4-(((2-chloro-[1,1′-biphenyl]-4-yl)methyl)amino)butyl)amino)ethyl)amino)benzo[c][2,6]naphthyridin-8-yl) acetamide;
5-((2-(4-(((2-Chloro-[1,1′-biphenyl]-4-yl)methyl)amino)butanamido)ethyl)amino)benzo[c][2,6]naphthyridine-8-carboxamide;
5-((3-(3-(((2-Chloro-[1,1′-biphenyl]-4-yl)methyl)amino)propoxy)propyl)amino)benzo[c][2,6]naphthyridine-8-carboxamide;
5-((2-(4-(((2-Chloro-[1,1′-biphenyl]-4-yl)methyl)amino)-N-methylbutanamido)ethyl)(methyl)amino)benzo[c][2,6]naphthyridine-8-carboxamide;
5-((3-(3-(((2-Chloro-[1,1′-biphenyl]-4-yl)methyl)amino)propanamido)propyl)amino)benzo[c][2,6]naphthyridine-8-carboxamide;
5-((3-(3-(((2-chloro-[1,1′-biphenyl]-4-yl)methyl)amino)-N-methylpropanamido)propyl)amino)benzo[c][2,6]naphthyridine-8-carboxamide;
5-((2-(4-(((2-Chloro-[1,1′-biphenyl]-4-yl)methyl)amino)butanamido)ethyl)(methyl)amino)benzo[c][2,6]naphthyridine-8-carboxamide;
5-((3-(3-(((2-Chloro-[1,1′-biphenyl]-4-yl)methyl)amino)propanamido)-3-methylbutyl)amino)benzo[c][2,6]naphthyridine-8-carboxamide;
5-((2-(4-(((2-Chloro-[1,1′-biphenyl]-4-yl)methyl)amino)-N-methylbutanamido)ethyl)amino)benzo[c][2,6]naphthyridine-8-carboxamide;
5-((2-(4-(2-(((2-Chloro-[1,1′-biphenyl]-4-yl)methyl)amino)ethyl)-1H-1,2,3-triazol-1-yl)ethyl)amino)benzo[c][2,6]naphthyridine-8-carboxamide;
5-((2-(5-(2-(((2-Chloro-[1,1′-biphenyl]-4-yl)methyl)amino)ethyl)-1,2,4-oxadiazol-3-yl)ethyl)amino)benzo[c][2,6]naphthyridine-8-carboxamide;
5-((3-(N-(2-(((2-chloro-[1,1′-biphenyl]-4-yl)methyl)amino)ethyl)sulfamoyl)propyl)amino)benzo[c][2,6]naphthyridine-8-carboxamide;
5-((2-(2-(2-(((2-chloro-[1,1′-biphenyl]-4-yl)methyl)amino)ethyl) oxazol-5-yl)ethyl)amino)benzo[c][2,6]naphthyridine-8-carboxamide;
5-((2-(3-(2-(((2-Chloro-[1,1′-biphenyl]-4-yl)methyl)amino)ethyl)-1,2,4-oxadiazol-5-yl)ethyl)amino)benzo[c][2,6]naphthyridine-8-carboxamide;
5-((2-(5-(2-(((2-Chloro-[1,1′-biphenyl]-4-yl)methyl)amino)ethyl)-4H-1,2,4-triazol-3-yl)ethyl)amino)benzo[c][2,6]naphthyridine-8-carboxamide;
5-((2-(4-(2-(((2-Chloro-[1,1′-biphenyl]-4-yl)methyl)amino)ethyl)-1H-imidazol-1-yl)ethyl)amino)benzo[c][2,6]naphthyridine-8-carboxamide;
5-((2-(2-(2-((3-Chloro-4-(trifluoromethoxy)benzyl)amino)ethyl) oxazol-5-yl)ethyl)amino)benzo[c][2,6]naphthyridine-8-carboxamide;
5-((2-(5-(2-((3-Chloro-4-(trifluoromethoxy)benzyl)amino)ethyl)-4H-1,2,4-triazol-3-yl)ethyl)amino)benzo[c][2,6]naphthyridine-8-carboxamide;
5-((2-(2-(2-(((2-Chloro-[1,1′-biphenyl]-4-yl)methyl)amino)ethyl)-1H-imidazol-5-yl)ethyl)amino)benzo[c][2,6]naphthyridine-8-carboxamide;
5-((2-(4-(2-((3-Chloro-4-(trifluoromethoxy)benzyl)amino)ethyl)-1H-imidazol-1-yl)ethyl)amino)benzo[c][2,6]naphthyridine-8-carboxamide;
5-((2-((4-((3-Chloro-4-(trifluoromethoxy)benzyl)amino)butyl)(methyl)amino)ethyl)amino)benzo[c][2,6]naphthyridine-8-carboxamide;
3-((3-Chloro-4-(trifluoromethoxy)benzyl)amino)-N-(3-((8-cyanobenzo[c][2,6]naphthyridin-5-yl)amino)propyl) propenamide;
5-((2-(4-(2-(((2-Chloro-[1,1′-biphenyl]-4-yl)methyl)amino)ethyl)-1H-1,2,3-triazol-1-yl)ethyl)amino)benzo[c][2,6]naphthyridine-8-carboxylic acid;
5-((3-(4-((((2-Chloro-[1,1′-biphenyl]-4-yl)methyl)amino)methyl)-1H-1,2,3-triazol-1-yl)propyl)amino)benzo[c][2,6]naphthyridine-8-carboxamide;
4-((3-(3-(((2-Chloro-[1,1′-biphenyl]-4-yl)methyl)amino)propanamido)propyl)amino)-3H-pyrazolo[3,4-c]quinoline-7-carboxylic acid;
5-((2-((4-((3-Chloro-4-(trifluoromethoxy)benzyl)amino)butyl)amino)ethyl)amino)benzo[c][2,6]naphthyridine-8-carboxylic acid;
6-(3-(3-(((2-Chloro-[1,1′-biphenyl]-4-yl)methyl)amino)propanamido)propyl)-5-oxo-5,6-dihydrobenzo[c][2,6]naphthyridine-8-carboxylic acid;
5-((2-((4-(((5-(Trifluoromethyl)-1H-indol-2-yl)methyl)amino)butyl)amino)ethyl)amino)benzo[c][2,6]naphthyridine-8-carboxylic acid;
5-((2-(((3-(((2-Chloro-[1,1′-biphenyl]-4-yl)methyl)amino)cyclobutyl)methyl)amino)ethyl)amino)benzo[c][2,6]naphthyridine-8-carboxamide;
5-((2-(3-(2-(((2-Chloro-[1,1′-biphenyl]-4-yl)methyl)amino)ethyl)ureido)ethyl)amino)benzo[c][2,6]naphthyridine-8-carboxamide;
5-((2-(3-(((2-Chloro-[1,1′-biphenyl]-4-yl)methyl)amino)propylsulfonamido)ethyl)amino)benzo[c][2,6]naphthyridine-8-carboxamide;
5-((3-(2-(((2-Chloro-[1,1′-biphenyl]-4-yl)methyl)amino)ethylsulfonamido)propyl)amino)benzo[c][2,6]naphthyridine-8-carboxamide;
N¹-(2-((8-(2H-tetrazol-5-yl)benzo[c][2,6]naphthyridin-5-yl)amino)ethyl)-N⁴-((2-chloro-[1,1′-biphenyl]-4-yl)methyl) butane-1,4-diamine;
5-((2-((4-((3-Chloro-4-(pyrimidin-5-yl)benzyl)amino)butyl)amino)ethyl)amino)benzo[c][2,6]naphthyridine-8-carboxamide;
5-((2-((4-(((6-Methyl-5-phenylpyridin-2-yl)methyl)amino)butyl)amino)ethyl)amino)benzo[c][2,6]naphthyridine-8-carboxamide;
5-((2-((4-((3-Chloro-4-(pyridin-2-yl)benzyl)amino)butyl)amino)ethyl)amino)benzo[c][2,6]naphthyridine-8-carboxamide;
5-((2-((4-(((2-Chloro-2′-(2-hydroxyethoxy)-[1,1′-biphenyl]-4-yl)methyl)amino)butyl)amino)ethyl)amino)benzo[c][2,6]naphthyridine-8-carboxamide;
5-(3-(3-((3-chloro-4-(trifluoromethoxy)benzyl)amino)propanamido)propyl)-4-oxo-4,5-dihydro-3H-pyrazolo[3,4-c]quinoline-7-carboxylic acid;
5-((4-(2-(((2-chloro-[1,1′-biphenyl]-4-yl)methyl)amino)ethoxy)butyl)amino)benzo[c][2,6]naphthyridine-8-carboxamide;
5-((2-(N-(3-(((2-chloro-[1,1′-biphenyl]-4-yl)methyl)amino)propyl)sulfamoyl)ethyl)amino)benzo[c][2,6]naphthyridine-8-carboxamide;
5-((2-(1-(2-(((2-Chloro-[1,1′-biphenyl]-4-yl)methyl)amino)ethyl)-1H-pyrazol-4-yl)ethyl)amino)benzo[c][2,6]naphthyridine-8-carboxamide;
5-((2-(1-(2-((3-Chloro-4-(trifluoromethoxy)benzyl)amino)ethyl)-1H-pyrazol-4-yl)ethyl)amino)benzo[c][2,6]naphthyridine-8-carboxamide;
5-((3-(3-((3-Chloro-4-(trifluoromethoxy)benzyl)amino)propoxy)propyl)amino)benzo[c][2,6]naphthyridine-8-carboxamide;
5-((4-(2-(((2-Chloro-[1,1′-biphenyl]-4-yl)methyl)amino)ethyl)phenethyl)amino)benzo[c][2,6]naphthyridine-8-carboxamide;
5-((4-(2-((3-Chloro-4-(trifluoromethoxy)benzyl)amino)ethoxy)butyl)amino)benzo[c][2,6]naphthyridine-8-carboxamide;
5-((3-(2-(((2-Chloro-[1,1′-biphenyl]-4-yl)methyl)amino)ethyl)phenethyl)amino)benzo[c][2,6]naphthyridine-8-carboxamide;
5-((3-((3-((3-Chloro-4-(trifluoromethoxy)benzyl)amino)propyl)amino)-3-oxopropyl)amino)benzo[c][2,6]naphthyridine-8-carboxamide;
5-((2-(4-((3-Chloro-4-(trifluoromethoxy)benzyl)amino)butanamido)ethyl)amino)benzo[c][2,6]naphthyridine-8-carboxamide;
5-((2-(4-((((2-Chloro-[1,1′-biphenyl]-4-yl)methyl)amino)methyl)piperidin-1-yl)ethyl)amino)benzo[c][2,6]naphthyridine-8-carboxamide;
5-((2-(5-(((2-Chloro-[1,1′-biphenyl]-4-yl)methyl)amino)-2H-indazol-2-yl)ethyl)amino)benzo[c][2,6]naphthyridine-8-carboxamide;
5-((2-(6-(((2-Chloro-[1,1′-biphenyl]-4-yl)methyl)amino)-1H-benzo[d]imidazol-2-yl)ethyl)amino)benzo[c][2,6]naphthyridine-8-carboxamide;
5-((2-((3-(((3-Chloro-4-(trifluoromethoxy)benzyl)amino)methyl)cyclobutyl)amino)ethyl)amino)benzo[c][2,6]naphthyrid ine-8-carboxamide;
5-((2-(3-(2-((3-Chloro-4-(trifluoromethoxy)benzyl)amino)ethyl)pyrrolidin-1-yl)ethyl)amino)benzo[c][2,6]naphthyridine-8-carboxamide;
5-((2-(3-(N-(3-chlorobenzyl)sulfamoyl)propoxy)ethyl)amino)benzo[c][2,6]naphthyridine-8-carboxamide;
(S)-5-((2-((4-((3-Chlorobenzyl)amino)pentyl)oxy)ethyl)amino)benzo[c][2,6]naphthyridine-8-carboxamide;
5-((4-(4-(((2-Chloro-[1,1′-biphenyl]-4-yl)methyl)amino)-1H−1,2,3-triazol-1-yl)butyl)amino)benzo[c][2,6]naphthyridine-8-carboxamide;
5-((3-(3-((3-Chlorobenzyl)amino)propanamido)propyl)amino)benzo[c][2,6]naphthyridine-8-carboxamide;
5-((3-(3-((3-Chloro-4-(trifluoromethoxy)benzyl)amino)propanamido)propyl)amino)benzo[c][2,6]naphthyridine-8-carboxamide;
5-((3-(3-((3-Chloro-4-cyclopropoxybenzyl)amino)propanamido)propyl)amino)benzo[c][2,6]naphthyridine-8-carboxamide;
5-((3-(3-((3-Chloro-4-cyclopropylbenzyl)amino)propanamido)propyl)amino)benzo[c][2,6]naphthyridine-8-carboxamide;
5-((3-(3-((3-chloro-4-(cyclopentyloxy)benzyl)amino)propanamido)propyl)amino)benzo[c][2,6]naphthyridine-8-carboxamide;
5-((3-(3-((3-chloro-4-cyclobutoxybenzyl)amino)propanamido)propyl)amino)benzo[c][2,6]naphthyridine-8-carboxamide;
5-((3-(3-(((6-Chloro-1H-indol-2-yl)methyl)amino)propanamido)propyl)amino)benzo[c][2,6]naphthyridine-8-carboxamide;
5-((3-(3-((3-Fluoro-4-(trifluoromethoxy)benzyl)amino)propanamido)propyl)amino)benzo[c][2,6]naphthyridine-8-carboxamide;
5-((3-(3-((3,4-Dichlorobenzyl)amino)propanamido)propyl)amino)benzo[c][2,6]naphthyridine-8-carboxamide;
5-((3-(3-((3,5-Dichlorobenzyl)amino)propanamido)propyl)amino)benzo[c][2,6]naphthyridine-8-carboxamide;
5-((3-(3-((3-Chloro-4-(trifluoromethyl)benzyl)amino)propanamido)propyl)amino)benzo[c][2,6]naphthyridine-8-carboxamide;
5-((3-(3-((3-Chloro-5-fluorobenzyl)amino)propanamido)propyl)amino)benzo[c][2,6]naphthyridine-8-carboxamide;
5-((3-(3-(((6-Chloro-1-methyl-1H-indol-2-yl)methyl)amino)propanamido)propyl)amino)benzo[c][2,6]naphthyridine-8-carboxamide;
5-((3-(3-(((4-Chloro-3-phenyl-1H-pyrazol-5-yl)methyl)amino)propanamido)propyl)amino)benzo[c][2,6]naphthyridine-8-carboxamide;
5-((3-(3-(((4-Chloro-1-methyl-3-phenyl-1H-pyrazol-5-yl)methyl)amino)propanamido)propyl)amino)benzo[c][2,6]naphthyridine-8-carboxamide;
5-((3-(3-(((6-(Trifluoromethyl)pyridin-3-yl)methyl)amino)propanamido)propyl)amino)benzo[c][2,6]naphthyridine-8-carboxamide;
5-((3-(3-(((5-Chloropyridin-3-yl)methyl)amino)propanamido)propyl)amino)benzo[c][2,6]naphthyridine-8-carboxamide;
5-((3-(3-(((5-(Trifluoromethyl)pyridin-2-yl)methyl)amino)propanamido)propyl)amino)benzo[c][2,6]naphthyridine-8-carboxamide;
5-((3-(3-((3-Chloro-4-cyclopentylbenzyl)amino)propanamido)propyl)amino)benzo[c][2,6]naphthyridine-8-carboxamide;
5-((3-(3-((3-(2-Hydroxyethyl)benzyl)amino)propanamido)propyl)amino)benzo[c][2,6]naphthyridine-8-carboxamide;
5-((3-(3-((3-(Hydroxymethyl)benzyl)amino)propanamido)propyl)amino)benzo[c][2,6]naphthyridine-8-carboxamide;
5-((3-(3-((3-(2-Hydroxyethoxy)benzyl)amino)propanamido)propyl)amino)benzo[c][2,6]naphthyridine-8-carboxamide;
5-((3-(3-((3-Cyanobenzyl)amino)propanamido)propyl)amino)benzo[c][2,6]naphthyridine-8-carboxamide;
5-((3-(3-((3-Chloro-4-cyclopentylbenzyl)amino)propanamido)propyl)amino)benzo[c][2,6]naphthyridine-8-carboxamide;
5-((3-(3-(((6-Methylpyridin-2-yl)methyl)amino)propanamido)propyl)amino)benzo[c][2,6]naphthyridine-8-carboxamide;
5-((3-(3-((3-Cyano-4-(trifluoromethoxy)benzyl)amino)propanamido)propyl)amino)benzo[c][2,6]naphthyridine-8-carboxamide;
5-((3-(3-((3-Chloro-5-(hydroxymethyl)benzyl)amino)propanamido)propyl)amino)benzo[c][2,6]naphthyridine-8-carboxamide;
5-((3-(3-((3-(Cyanomethyl)benzyl)amino)propanamido)propyl)amino)benzo[c][2,6]naphthyridine-8-carboxamide;
5-((3-(3-((3-cyano-4-cyclobutoxybenzyl)amino)propanamido)propyl)amino)benzo[c][2,6]naphthyridine-8-carboxamide;
5-((3-(3-(((2-cyano-[1,1′-biphenyl]-4-yl)methyl)amino)propanamido)propyl)amino)benzo[c][2,6]naphthyridine-8-carboxamide;
5-((3-(3-(((6-(trifluoromethoxy) quinolin-2-yl)methyl)amino)propanamido)propyl)amino)benzo[c][2,6]naphthyridine-8-carboxamide;
5-((3-(3-((3-cyano-4-cyclopropylbenzyl)amino)propanamido)propyl)amino)benzo[c][2,6]naphthyridine-8-carboxamide;
5-((3-(3-(((6-Cyclopropylpyridin-3-yl)methyl)amino)propanamido)propyl)amino)benzo[c][2,6]naphthyridine-8-carboxamide;
5-((3-(3-(((5-Cyclopropylpyridin-2-yl)methyl)amino)propanamido)propyl)amino)benzo[c][2,6]naphthyridine-8-carboxamide;
5-((3-(3-((3,5-Difluoro-4-(trifluoromethoxy)benzyl)amino)propanamido)propyl)amino)benzo[c][2,6]naphthyridine-8-carboxamide;
5-((3-(3-((3-Chloro-5-(cyanomethyl)benzyl)amino)propanamido)propyl)amino)benzo[c][2,6]naphthyridine-8-carboxamide;
5-((2-(2-(2-((3-Chloro-4-(trifluoromethoxy)benzyl)amino)ethoxy)ethoxy)ethyl)amino)benzo[c][2,6]naphthyridine-8-carboxamide;
5-((2-(4-(2-((3-Chloro-4-(trifluoromethoxy)benzyl)amino)ethyl)-1H−1,2,3-triazol-1-yl)ethyl)amino)benzo[c][2,6]naphthyridine-8-carboxamide;
5-((2-(4-(2-(((6-Methyl-5-phenylpyridin-2-yl)methyl)amino)ethyl)-1H-1,2,3-triazol-1-yl)ethyl)amino)benzo[c][2,6]naphthyridine-8-carboxamide;
5-((2-(4-(2-((3-chlorobenzyl)amino)ethyl)-1H-1,2,3-triazol-1-yl)ethyl)amino)benzo[c][2,6]naphthyridine-8-carboxamide;
5-((2-(4-(2-(((5-(Trifluoromethyl)-1H-indol-2-yl)methyl)amino)ethyl)-1H-1,2,3-triazol-1-yl)ethyl)amino)benzo[c][2,6]naphthyridine-8-carboxamide;
5-((2-(4-(2-((3-chloro-5-fluorobenzyl)amino)ethyl)-1H-1,2,3-triazol-1-yl)ethyl)amino)benzo[c][2,6]naphthyridine-8-carboxamide;
5-((2-(4-(2-((3,5-dichlorobenzyl)amino)ethyl)-1H-1,2,3-triazol-1-yl)ethyl)amino)benzo[c][2,6]naphthyridine-8-carboxamide;
5-((2-(4-(2-(((6-Chloro-1H-indol-2-yl)methyl)amino)ethyl)-1H-1,2,3-triazol-1-yl)ethyl)amino)benzo[c][2,6]naphthyridine-8-carboxamide;
5-((2-(4-(2-((3-Chloro-4-cyclobutoxybenzyl)amino)ethyl)-1H-1,2,3-triazol-1-yl)ethyl)amino)benzo[c][2,6]naphthyridine-8-carboxamide;
5-((2-(4-(2-((3-Chloro-4-cyclopropylbenzyl)amino)ethyl)-1H-1,2,3-triazol-1-yl)ethyl)amino)benzo[c][2,6]naphthyridine-8-carboxamide;
5-((2-(4-(2-((3-fluoro-4-(trifluoromethoxy)benzyl)amino)ethyl)-1H-1,2,3-triazol-1-yl)ethyl)amino)benzo[c][2,6]naphthyridine-8-carboxamide;
5-((2-(4-(2-(((2-Chloro-2′-(2-hydroxyethoxy)-[1,1′-biphenyl]-4-yl)methyl)amino)ethyl)-1H-1,2,3-triazol-1-yl)ethyl)amino)benzo[c][2,6]naphthyridine-8-carboxamide;
5-((2-(4-(2-(((5-Chloro-6-phenylpyridin-3-yl)methyl)amino)ethyl)-1H-1,2,3-triazol-1-yl)ethyl)amino)benzo[c][2,6]naphthyridine-8-carboxamide;
5-((2-(4-(2-(((1-Methyl-5-(trifluoromethyl)-1H-indol-2-yl)methyl)amino)ethyl)-1H-1,2,3-triazol-1-yl)ethyl)amino)benzo[c][2,6]naphthyridine-8-carboxamide;
5-((3-(3-((3-Chloro-4-cyclobutoxybenzyl)amino)propanamido)propyl)amino)benzo[c][2,6]naphthyridine-8-carboxylic acid;
5-((3-(3-((3-Chloro-4-cyclobutoxybenzyl)amino)-N-methylpropanamido)propyl)amino)benzo[c][2,6]naphthyridine-8-carboxamide;
5-((3-(3-((3-Chloro-4-(trifluoromethoxy)benzyl)amino)propanamido)propyl)amino)benzo[c][2,6]naphthyridine-8-carboxylic acid;
5-((3-(3-((3-Chloro-4-(trifluoromethoxy)benzyl)amino)propoxy)propyl)amino)benzo[c][2,6]naphthyridine-8-carboxylic acid;
5-((3-((3-((3-Chloro-4-(trifluoromethoxy)benzyl)amino)propyl)amino)-3-oxopropyl)amino)benzo[c][2,6]naphthyridine-8-carboxylic acid;
5-((4-(2-((3-Chloro-4-(trifluoromethoxy)benzyl)amino)ethoxy)butyl)amino)benzo[c][2,6]naphthyridine-8-carboxylic acid;
5-((4-((2-((3-Chloro-4-(trifluoromethoxy)benzyl)amino)ethyl)amino)-4-oxobutyl)amino)benzo[c][2,6]naphthyridine-8-carboxylic acid;
5-((2-(4-((3-Chloro-4-(trifluoromethoxy)benzyl)amino)butanamido)ethyl)amino)benzo[c][2,6]naphthyridine-8-carboxylic acid;
5-(2-(4-((3-Chloro-4-(trifluoromethoxy)benzyl)amino)butoxy)ethyl)-4-oxo-4,5-dihydro-3H-pyrazolo[3,4-c]quinoline-7-carboxylic acid;
5-(4-(3-((3-Chloro-4-(trifluoromethoxy)benzyl)amino)propoxy)piperidin-1-yl)benzo[c][2,6]naphthyridine-8-carboxylic acid;
5-(4-(4-((3-Chloro-4-(trifluoromethoxy)benzyl)amino)butyl)piperazin-1-yl)benzo[c][2,6]naphthyridine-8-carboxylic acid;
(R)-5-(3-(3-((3-Chloro-4-(trifluoromethoxy)benzyl)amino)propoxy) pyrrolidin-1-yl)benzo[c][2,6]naphthyridine-8-carboxylic acid;
(S)-5-(3-(3-((3-Chloro-4-(trifluoromethoxy)benzyl)amino)propoxy) pyrrolidin-1-yl)benzo[c][2,6]naphthyridine-8-carboxylic acid;
N-(3-((8-(2H-tetrazol-5-yl)benzo[c][2,6]naphthyridin-5-yl)amino)propyl)-3-((3-chloro-4-cyclobutoxybenzyl)amino) propenamide;
N-(3-((8-(2H-tetrazol-5-yl)benzo[c][2,6]naphthyridin-5-yl)amino)propyl)-3-((3-chloro-4-(trifluoromethoxy)benzyl)amino) propenamide;
N-(2-(4-(2-(((2-chloro-[1,1′-biphenyl]-4-yl)methyl)amino)ethyl)-1H-1,2,3-triazol-1-yl)ethyl)-8-(2H-tetrazol-5-yl)benzo[c][2,6]naphthyridin-5-amine;
5-(3-(3-((3-Chloro-4-(trifluoromethoxy)benzyl)amino)propanamido)propyl)-4-oxo-4,5-dihydro-3H-pyrazolo[3,4-c]quinoline-7-carboxamide;
5-((2-((3-(((2-Chloro-[1,1′-biphenyl]-4-yl)methyl)amino)cyclobutyl)methoxy)ethyl)amino)benzo[c][2,6]naphthyridine-8-carboxamide;
5-((2-((5-((3-Chlorophenyl)amino)pentyl)oxy)ethyl)amino)benzo[c][2,6]naphthyridine-8-carboxamide;
5-(3-(4-(((5-Cyclobutoxy-1H-indol-2-yl)methyl)amino)butoxy) azetidin-1-yl)benzo[c][2,6]naphthyridine-8-carboxamide;
(S)-5-(3-(3-((3-chloro-4-(trifluoromethoxy)benzyl)amino)propoxy) pyrrolidin-1-yl)benzo[c][2,6]naphthyridine-8-carboxamide;
5-(4-(4-((3,5-Difluoro-4-(trifluoromethoxy)benzyl)amino)-1H-1,2,3-triazol-1-yl)butoxy)benzo[c][2,6]naphthyridine-8-carboxylic acid;
5-(2-(4-(2-((3,5-Difluoro-4-(trifluoromethoxy)benzyl)amino)ethyl)-1H-1,2,3-triazol-1-yl)ethoxy)benzo[c][2,6]naphthyridine-8-carboxylic acid;
5-(2-((3-((3,5-Difluoro-4-(trifluoromethoxy)benzyl)amino)cyclobutyl)methoxy)ethoxy)benzo[c][2,6]naphthyridine-8-carboxylic acid; or
(S)-5-(2-((4-((3,5-difluoro-4-(trifluoromethoxy)benzyl)amino)pentyl)oxy)ethoxy)benzo[c][2,6]naphthyridine-8-carboxylic acid.

24. A pharmaceutical composition comprising a compound according to any one of claims 1 to 23, or a pharmaceutically acceptable salt, hydrate or solvate thereof, and a pharmaceutically acceptable excipient.

25. A compound according to any one of claims 1 to 23, or a pharmaceutically acceptable salt of solvate thereof, or a pharmaceutical composition according to claim 24 for use in:
(i) therapy:
(ii) the treatment of a disease or condition in which CK2α activity is implicated;
(iii) the treatment of a disease or condition associated with aberrant activity of CK2α;
(iv) the treatment of proliferative disorders (e.g. cancer or benign neoplasms), viral infections, an inflammatory disease or condition, diabetes, vascular and ischemic disorders, neurodegenerative disorders and/or the regulation of circadian rhythm;
(v) the treatment of a cancer; and/or
(vi) the treatment of a viral infection.
